(19) United States
(12) Patent Application Publication
(10) Pub. No.: US 2026/0102422 A1
(43) Pub. Date: Apr. 16, 2026

(54) 4'-HALOGEN CONTAINING NUCLEOTIDE AND NUCLEOSIDE THERAPEUTIC COMPOSITIONS AND USES RELATED THERETO

(71) Applicant: Emory University, Atlanta, GA (US)

(72) Inventors: George R. PAINTER, Atlanta, GA (US); David PERRYMAN, Decatur, GA (US); Gregory R. BLUEMLING, Decatur, GA (US)

(21) Appl. No.: 19/292,470

(22) Filed: Aug. 6, 2025

Related U.S. Application Data
(63) Continuation of application No. 17/766,644, now abandoned.
(60) Provisional application No. 62/912,165, filed on Oct. 8, 2019.

(51) Int. Cl. A61K 31/7072; A61K 45/06; A61P 31/14

(52) U.S. Cl. CPC A61K 31/7072; A61K 45/06; A61P 31/14

(57) ABSTRACT

Disclosed are halogen containing nucleotide and nucleoside therapeutic compositions and uses related thereto. In certain embodiments, the disclosure relates to the treatment or prophylaxis of viral infections. Such viral infections can include tongaviridae, bunyaviridae, arenaviridae, coronaviridae, flaviviridae, picornaviridae, Eastern, Western, and Venezuelan Equine Encephalitis (EEE, WEE and VEE, respectively), Chikungunya fever (CHIK), Ebola, Influenza, RSV, and Zika virus infections.

### STATEMENT ACKNOWLEDGING OF GOVERNMENT SUPPORT

This invention was made with government support under Contract Nos. HDTRA1-13-C-0072 and HDTRA1-15-C-0075 awarded by the Department of Defense (DTRA), and Grant No. HHSN272201500008C awarded by the National Institutes of Health. The government has certain rights in the invention.

### FIELD

This disclosure relates to halogen containing nucleotide and nucleoside therapeutic compositions and uses related thereto. In certain embodiments, the disclosure relates to the treatment or prophylaxis of viral infections, for example, tongaviridae, bunyaviridae, arenaviridae, coronaviridae, flaviviridae, picornaviridae, Eastern, Western, and Venezuelan Equine Encephalitis (EEE, WEE and VEE, respectively), Chikungunya fever (CHIK), Ebola, Influenza, RSV, and Zika virus infections.

### BACKGROUND

The causative agents for Eastern, Western, and Venezuelan Equine Encephalitis (EEE, WEE and VEE, respectively) and Chikungunya fever (CHIK) are vector-borne viruses (family Togaviridae, genus Alphavirus) that can be transmitted to humans through mosquito bites. The equine encephalitis viruses are CDC Category B pathogens, and the CHIK virus is Category C₆. There is considerable concern about the use of virulent strains of VEE virus, delivered via aerosol, as a bioweapon against warfighters. Animal studies have demonstrated that infection with VEE virus by aerosol exposure rapidly leads to a massive infection of the brain, with high mortality and morbidity. See Roy et al., Pathogenesis of aerosolized Eastern equine encephalitis virus infection in guinea pigs. Virol J, 2009, 6:170.

Coronaviruses are enveloped positive-sense RNA viruses that cause a large percentage of respiratory illness in humans. The two previous coronaviruses to emerge and cause human illness were SARS and MERS. There were more than 8,000 human cases of SARS with 774 deaths. Since 2012, there have been more than 2,500 cases of MERS with 919 deaths. In 2019 a new coronavirus, 2019-nCoV and now known as SARS-CoV-2, was discovered in humans in Wuhan, China. Reports from early February 2020 indicate more than 28,000 people have been infected with the novel coronavirus with more than 560 deaths documented. In addition, human-to-human transmission of 2019-nCov has been documented. Analysis of a single completed full-genome sequence revealed 2019-nCov belongs to betacoronavirus but is divergent from SARS and MERS. The 2019-nCoV is a highly pathogenic human pathogen that relatively little is known about. SARS-CoV-2/2019-nCoV, causes disease referred to a COVID-19. COVID-19 can include severe respiratory disease in humans and appears to also cause neurological disease that includes dizziness, impaired consciousness, acute cerebrovascular disease, epilepsy, hyposmia, hypopsia, and neuralgia (medRxiv, 2020, 1-26). SARS-CoV-2 entry into the CNS may be promoted through viral interaction with ACE2 receptors after dissemination of the virus in the systemic circulation or across the cribriform plate. Additional studies are needed to further characterize the virus and to identify ways to prevent and treat disease.

What are needed are new compounds and treatments for viral infections. The compounds and methods disclosed herein addressed these needs.

References cited herein are not an admission of prior art.

### SUMMARY

This disclosure relates to halogen, e.g. 4′-halogen, containing nucleotide and nucleoside therapeutic compositions and uses related thereto. Included are nucleosides optionally conjugated to a phosphorus oxide or salts thereof, prodrugs or conjugate compounds or salts thereof comprising an amino acid ester, lipid or a sphingolipid or derivative linked by a phosphorus oxide to a nucleotide or nucleoside. In certain embodiments, the disclosure relates to a compound having Formula A,

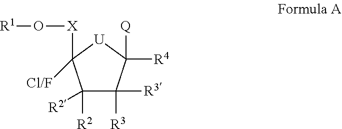

or a pharmaceutically acceptable salt, derivative, or prodrug thereof, as defined herein.

In certain embodiments, the disclosure contemplates derivatives of compounds disclosed herein, such as those containing one or more, the same or different, substituents.

In certain embodiments, the disclosure contemplates pharmaceutical compositions comprising a pharmaceutically acceptable excipient and a compound disclosed herein. In certain embodiments, the pharmaceutical composition is in the form of a tablet, capsule, pill, or aqueous buffer, such as a saline or phosphate buffer.

In certain embodiments, the disclosed pharmaceutical compositions can comprise a compound disclosed herein and a propellant. In certain embodiments, the propellant is an aerosolizing propellant such as compressed air, ethanol, nitrogen, carbon dioxide, nitrous oxide, hydrofluoroalkanes (HFAs), 1,1,1,2,-tetrafluoroethane, 1,1,1,2,3,3,3-heptafluoropropane or combinations thereof.

In certain embodiments, the disclosure contemplates a pressurized or unpressurized container comprising a compound or pharmaceutical composition as described herein. In certain embodiments, the container is a manual pump spray, inhaler, meter-dosed inhaler, dry powder inhaler, nebulizer, vibrating mesh nebulizer, jet nebulizer, or ultrasonic wave nebulizer.

In certain embodiments, the disclosure relates to methods of increasing bioavailability for treating or preventing a viral infection comprising administering an effective amount of a compound or pharmaceutical composition disclosed herein to a subject in need thereof.

In certain embodiments, the disclosure relates to methods of treating or preventing a viral infection comprising administering an effective amount of a compound or pharmaceutical composition disclosed herein to a subject in need thereof. In certain embodiments, the viral infection is tongaviridae, bunyaviridae, arenaviridae, coronaviridae, flaviviridae, picornaviridae, Zika virus infection, Eastern, Western, and Venezuelan Equine Encephalitis (EEE, WEE and VEE, respectively), Chikungunya fever (CHIK), Ebola, Influenza, and RSV.

In certain embodiments, the compound or pharmaceutical composition is administered orally, intravenously, or through the lungs, i.e., pulmonary administration.

In certain embodiments, the disclosure relates to the use of a compound as described herein in the production of a medicament for the treatment or prevention of a viral infection, such as Eastern, Western, and Venezuelan Equine Encephalitis (EEE, WEE and VEE, respectively), Chikungunya fever (CHIK), Ebola, Influenza, RSV, or Zika virus infection.

In certain embodiments, the disclosure relates to methods of making compounds disclosed herein by mixing starting materials and reagents disclosed herein under conditions such that the compounds are formed.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

FIG. 1 shows stability of EIDD-02749 in methanol.

FIG. 2 shows stability of EIDD-02749 in water.

FIG. 3 shows stability of EIDD-02749 in 0.1 N HCl.

FIG. 4 shows stability of EIDD-02749 in PBS at pH 7.4.

FIG. 5 shows stability of EIDD-02749 in pH 9 buffer.

FIG. 6 shows triphosphate concentrations in Huh-7 cells incubated with EIDD-02749 or a prodrug.

FIG. 7 shows triphosphate concentrations in Vero cells incubated with EIDD-02749 or a prodrug.

FIG. 8 shows plasma PK curves from CD-1 mice that received a single dose of EIDD-02749 at 50, 150, and 500 mg/kg PO or 10 mg/kg ip.

FIG. 9 shows the change in body weight of AG129 mice dosed with 10, 30, and 100 mg/kg EIDD-02749 QD for 10 days.

FIG. 10 shows plasma PK curves from CD-1 mice that received a single dose of EIDD-02749 at 1.5, 5, 15, 50, 150, and 500 mg/kg PO or 10 mg/kg ip.

FIG. 11 shows plasma PK curves from ferrets that received a single dose of EIDD-02749 at 15 and 50 mg/kg PO.

FIG. 12 shows plasma PK curves from Guinea pigs that received a single dose of EIDD-02749 at 5 and 50 mg/kg PO and 5 mg/kg i.v.

FIG. 13 shows concentrations of EIDD-02749 (ribonucleoside), EIDD-02986 (5′-monophosphate), and EIDD-02991 (5′-triphosphate) in tissues from CD-1 mice that received a single oral dose of 5, 50, and 500 mg/kg EIDD-02749.

FIG. 14 shows concentrations of EIDD-02749 (ribonucleoside), EIDD-02986 (5′-monophosphate), and EIDD-02991 (5′-triphosphate) in tissues from Guinea pigs that received a single oral dose of 50 mg/kg EIDD-02749.

FIG. 15 shows the survival of AG129 mice infected with Tacaribe virus and treated with EIDD-02749 starting 2 hours before infection.

FIG. 16 shows Day 9 viral titers in serum (Graph A); liver tissue (Graph B); spleen tissue (Graph C); and brain tissue (Graph D) tissues from AG129 mice infected with Tacaribe virus and treated with EIDD-02749.

FIG. 17 shows the survival of AG129 mice infected with Tacaribe virus treated with EIDD-02749 starting at 2 hours, 1 Day, 3 Days, 5 Days, and 7 Days post infection.

FIG. 18 shows Day 9 viral titers in tissues from AG129 mice infected with Tacaribe virus and treated with EIDD-02749 starting at 2 hours, 1 Day, 3 Days, 5 Days, and 7 Days post infection.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features, which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

This disclosure relates to 4′-halogen containing nucleotide and nucleoside therapeutic compositions and uses related thereto. In certain embodiments, the disclosure relates to nucleosides optionally conjugated to a phosphorus oxide or salts thereof. In certain embodiments, the disclosure relates to conjugate compounds or salts thereof comprising an amino acid ester, a lipid or a sphingolipid or derivative linked by a phosphorus oxide to a nucleotide or nucleoside. In certain embodiments, the disclosure contemplates pharmaceutical compositions comprising these compounds for uses in treating infectious diseases, viral infections, and cancer.

In certain embodiments, the disclosure relates to phosphorus oxide prodrugs of 4′-halogen containing nucleosides for the treatment of positive-sense and negative-sense RNA viral infections through targeting of the virally encoded RNA-dependent RNA polymerase (RdRp). This disclosure also provides the general use of lipids and sphingolipids to deliver nucleoside analogs for the treatment of infectious disease and cancer.

In certain embodiments, the disclosure relates to conjugate compounds or salts thereof comprising a sphingolipid or derivative linked by a phosphorus oxide to a nucleotide or nucleoside. In certain embodiments, the phosphorus oxide is a phosphate, phosphonate, polyphosphate, or polyphosphonate, wherein the phosphate, phosphonate or a phosphate in the polyphosphate or polyphosphonate is optionally a phosphorothioate or phosphoroamidate. In certain embodiments, the lipid or sphingolipid is covalently bonded to the phosphorus oxide through an amino group or a hydroxyl group.

The nucleotide or nucleoside comprises a heterocycle comprising two or more nitrogen heteroatoms, wherein the substituted heterocycle is optionally substituted with one or more, the same or different alkyl, halogen, or cycloalkyl.

In certain embodiments, the sphingolipid is saturated or unsaturated 2-aminoalkyl or 2-aminooctadecane optionally substituted with one or more substituents. In certain embodiments, the sphingolipid derivative is saturated or unsaturated 2-aminooctadecane-3-ol optionally substituted with one or more substituents. In certain embodiments, the sphingolipid derivative is saturated or unsaturated 2-aminooctadecane-3,5-diol optionally substituted with one or more substituents.

In certain embodiments, the disclosure contemplates pharmaceutical compositions comprising any of the compounds disclosed herein and a pharmaceutically acceptable excipient. In certain embodiments, the pharmaceutical composition is in the form of a pill, capsule, tablet, or saline buffer comprising a saccharide. In certain embodiments, the composition may contain a second active agent such as a pain reliever, anti-inflammatory agent, non-steroidal anti-inflammatory agent, anti-viral agent, anti-biotic, or anti-cancer agent.

In certain embodiments, the disclosure relates to methods of treating or preventing an infection comprising administering an effective amount of a compound disclosed herein to a subject in need thereof. Typically, the subject is diagnosed with or at risk of an infection from a virus, bacteria, fungi, protozoa, or parasite.

In certain embodiments, the disclosure relates the methods of treating a viral infection comprising administering an effective amount of a pharmaceutical composition disclosed herein to a subject in need thereof. In certain embodiments, the subject is a mammal, for example, a human. In certain embodiments, the subject is diagnosed with a chronic viral infection. In certain embodiments, administration is under conditions such that the viral infection is no longer detected. In certain embodiments, the subject is diagnosed with a RNA virus, DNA virus, or retroviruses. In certain embodiments, the subject is diagnosed with a virus that is a double stranded DNA virus, sense single stranded DNA virus, double stranded RNA virus, sense single stranded RNA virus, antisense single stranded RNA virus, sense single stranded RNA retrovirus or a double stranded DNA retrovirus.

In certain embodiments, the subject is diagnosed with influenza A virus including subtype H1N1, H3N2, H7N9, or H5N1, influenza B virus, influenza C virus, rotavirus A, rotavirus B, rotavirus C, rotavirus D, rotavirus E, human coronavirus, SARS coronavirus, MERS coronavirus, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, coxsackie B virus, norovirus, Rubella virus, lymphocytic choriomeningitis virus (LCMV), chikungunya, Eastern equine encephalitis virus (EEEV), Western equine encephalitis virus (WEEV), Venezuelan equine encephalitis virus (VEEV), Ross River virus, Barmah Forest virus, measles virus, mumps virus, respiratory syncytial virus, rinderpest virus, California encephalitis virus, hantavirus, rabies virus, ebola virus, marburg virus, herpes simplex virus-1 (HSV-1), herpes simplex virus-2 (HSV-2), varicella zoster virus (VZV), Epstein-Barr virus (EBV), cytomegalovirus (CMV), herpes lymphotropic virus, roseolovirus, or Kaposi's sarcoma-associated herpesvirus, hepatitis A, hepatitis B, hepatitis D, hepatitis E or human immunodeficiency virus (HIV).

In certain embodiments, the subject is diagnosed with influenza A virus including subtypes H1N1, H3N2, H7N9, H5N1 (low path), and H5N1 (high path) influenza B virus, influenza C virus, rotavirus A, rotavirus B, rotavirus C, rotavirus D, rotavirus E, SARS coronavirus, MERS-CoV, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, coxsackie B virus, norovirus, Rubella virus, lymphocytic choriomeningitis virus (LCMV), measles virus, mumps virus, respiratory syncytial virus, parainfluenza viruses 1 and 3, rinderpest virus, chikungunya, eastern equine encephalitis virus (EEEV), Venezuelan equine encephalitis virus (VEEV), western equine encephalitis virus (WEEV), California encephalitis virus, Rift Valley fever virus (RVFV), heartland virus, La Crosse virus, Marpol virus, Severe fever thrombocytopenia syndrome virus, Pichinde virus, hantavirus, Tacaribe virus, Junin, rabies virus, ebola virus, marburg virus, adenovirus, herpes simplex virus-1 (HSV-1), herpes simplex virus-2 (HSV-2), varicella zoster virus (VZV), Epstein-Barr virus (EBV), cytomegalovirus (CMV), herpes lymphotropic virus, roseolovirus, or Kaposi's sarcoma-associated herpesvirus, hepatitis A, hepatitis B, hepatitis D, hepatitis E or human immunodeficiency virus (HIV).

In certain embodiment, the disclosure relates to uses of compounds disclosed herein in the production or manufacture of a medicament for the treatment or prevention of an infectious disease, viral infection, or cancer.

In certain embodiments, the disclosure relates to derivatives of compounds disclosed herein or any of the formula.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

In certain embodiments, a pharmaceutical agent, which may be in the form of a salt or prodrug, is administered in methods disclosed herein that is specified by a weight. This refers to the weight of the recited compound. If in the form of a salt or prodrug, then the weight is the molar equivalent of the corresponding salt or prodrug.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

Prior to describing the various embodiments, the following definitions are provided and should be used unless otherwise indicated.

As used herein, the term “deuterium” or “D” refers to the isotopic abundance of D relative to H (hydrogen) is at least 50%, at least 75%, or at least 90%.

As used herein, the term “phosphorus oxide” refers to any variety of chemical moieties that contain a phosphorus-oxygen (P—O or P═O) bond. When used as linking groups herein, the joined molecules may bond to oxygen or directly to the phosphorus atoms. The term is intended to include, but are not limited to phosphates, in which the phosphorus is typically bonded to four oxygens and phosphonates, in which the phosphorus is typically bonded to one carbon and three oxygens. A “polyphosphate” generally refers to phosphates linked together by at least one phosphorus-oxygen-phosphorus (P—O—P) bond. A “polyphosphonate” refers to a polyphosphate that contains at least one phosphorus-carbon (C—P—O—P) bond. In addition to containing phosphorus-oxygen bond, phosphorus oxides may contain a phosphorus-thiol (P—S or P═S) bond and/or a phosphorus-amine (P—N) bond, respectively referred to as phosphorothioate or phosphoroamidate. In phosphorus oxides, the oxygen atom may form a double or single bond to the phosphorus or combinations, and the oxygen may further bond with other atoms such as carbon or may exist as an anion which is counter balanced with a cation, e.g., metal or quaternary amine. “Subject” refers any animal, preferably a human patient, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent can be administered prior to, together with, or after the additional treatment, or a combination thereof.

As used herein, “alkyl” means a straight or branched chain saturated hydrocarbon moieties such as those containing from 1 to 10 carbon atoms. A “higher alkyl” refers to saturated hydrocarbon having 11 or more carbon atoms. A “C₆-C₁₆” refers to an alkyl containing 6 to 16 carbon atoms. Likewise a “C₆-C₂₂” refers to an alkyl containing 6 to 22 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like.

As used herein, the term “alkenyl” refers to unsaturated, straight or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond.

Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure-CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C.

As used herein, the term “alkynyl” represents straight or branched hydrocarbon moieties containing a triple bond. Unless otherwise specified, C₂-C₂₄ (e.g., C₂-C₂₄, C₂-C₂₀, C₂-C₁₈, C₂-C₁₆, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulfur which can be saturated or unsaturated (but not aromatic), monocyclic or polycyclic, and wherein the nitrogen and sulfur heteroatoms can be optionally oxidized, and the nitrogen heteroatom can be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

The term “aryl” refers to aromatic homocyclic (i.e., hydrocarbon) mono-, bi- or tricyclic ring-containing groups preferably having 6 to 12 members such as phenyl, naphthyl and biphenyl. Phenyl is a preferred aryl group. The term “substituted aryl” refers to aryl groups substituted with one or more groups, preferably selected from alkyl, substituted alkyl, alkenyl (optionally substituted), aryl (optionally substituted), heterocyclo (optionally substituted), halo, hydroxy, alkoxy (optionally substituted), aryloxy (optionally substituted), alkanoyl (optionally substituted), aroyl, (optionally substituted), alkylester (optionally substituted), arylester (optionally substituted), cyano, nitro, amino, substituted amino, amido, lactam, urea, urethane, sulfonyl, and, the like, where optionally one or more pair of substituents together with the atoms to which they are bonded form a 3 to 7 member ring.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems can, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems can be aromatic, non-aromatic or mixtures of aromatic and non-aromatic rings. Heterocycle includes heterocarbocycles, heteroaryls, and the like.

“Alkylthio” refers to an alkyl group as defined above with the indicated number of carbon atoms attached through a sulfur bridge. An example of an alkylthio is methylthio, (i.e., —S—CH₃).

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above with the indicated number of carbon atoms attached through an amino bridge. An example of an alkylamino is methylamino, (i.e., —NH—CH₃).

“Alkanoyl” refers to an alkyl as defined above with the indicated number of carbon atoms attached through a carbonyl bride (i.e., —(C═O)alkyl).

“Alkylsulfonyl” refers to an alkyl as defined above with the indicated number of carbon atoms attached through a sulfonyl bridge (i.e., —S(═O)₂alkyl) such as mesyl and the like, and “Arylsulfonyl” refers to an aryl attached through a sulfonyl bridge (i.e., —S(═O)₂aryl).

“Alkylsulfamoyl” refers to an alkyl as defined above with the indicated number of carbon atoms attached through a sulfamoyl bridge (i.e., —NHS(═O)₂alkyl), and an “Arylsulfamoyl” refers to an alkyl attached through a sulfamoyl bridge (i.e., —NHS(═O)₂aryl).

“Alkylsulfinyl” refers to an alkyl as defined above with the indicated number of carbon atoms attached through a sulfinyl bridge (i.e. —S(═O)alkyl).

The terms “cycloalkyl” and “cycloalkenyl” refer to mono-, bi-, or tri homocyclic ring groups of 3 to 15 carbon atoms which are, respectively, fully saturated and partially unsaturated. The term “cycloalkenyl” includes bi- and tricyclic ring systems that are not aromatic as a whole, but contain aromatic portions (e.g., fluorene, tetrahydronapthalene, dihydroindene, and the like). The rings of multi-ring cycloalkyl groups can be either fused, bridged and/or joined through one or more spiro unions. The terms “substituted cycloalkyl” and “substituted cycloalkenyl” refer, respectively, to cycloalkyl and cycloalkenyl groups substituted with one or more groups, preferably selected from aryl, substituted aryl, heterocyclo, substituted heterocyclo, carbocyclo, substituted carbocyclo, halo, hydroxy, alkoxy (optionally substituted), aryloxy (optionally substituted), alkylester (optionally substituted), arylester (optionally substituted), alkanoyl (optionally substituted), aryol (optionally substituted), cyano, nitro, amino, substituted amino, amido, lactam, urea, urethane, sulfonyl, and the like.

The terms “halogen” and “halo” refer to fluorine, chlorine, bromine, and iodine.

The term “substituted” refers to a molecule wherein at least one hydrogen atom is replaced with a substituent. When substituted, one or more of the groups are “substituents.” The molecule can be multiply substituted. In the case of an oxo substituent (“=₀”), two hydrogen atoms are replaced. Example substituents within this context can include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO₂Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context can be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution with an additional group is optional and therefore it is possible for the designated atom to be unsubstituted. Thus, by use of the term “optionally substituted” the disclosure includes examples where the group is substituted and examples where it is not.

Examples of prodrugs that can be used to improve bioavailability include esters, optionally substituted esters, branched esters, optionally substituted branched esters, carbonates, optionally substituted carbonates, carbamates, optionally substituted carbamates, thioesters, optionally substituted thioesters, branched thioesters, optionally substituted branched thioesters, thiocarbonates, optionally substituted thiocarbonates, S-thiocarbonate, optionally substituted S-thiocarbonate, dithiocarbonates, optionally substituted dithiocarbonates, thiocarbamates, optionally substituted thiocarbamates, oxymethoxycarbonyl, optionally substituted oxymethoxycarbonyl, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, sulfenyl, optionally substituted sulfenyl, imidate, optionally substituted imidate, hydrazonate, optionally substituted hydrazonate, oximyl, optionally substituted oximyl, imidinyl, optionally substituted imidinyl, imidyl, optionally substituted imidyl, aminal, optionally substituted aminal, hemiaminal, optionally substituted hemiaminal, acetal, optionally substituted acetal, hemiacetal, optionally substituted hemiacetal, carbonimidate, optionally substituted carbonimidate, thiocarbonimidate, optionally substituted thiocarbonimidate, carbonimidyl, optionally substituted carbonimidyl, carbamimidate, optionally substituted carbamimidate, carbamimidyl, optionally substituted carbamimidyl, thioacetal, optionally substituted thioacetal, S-acyl-2-thioethyl, optionally substituted S-acyl-2-thioethyl, bis-(acyloxybenzyl)esters, optionally substituted bis-(acyloxybenzyl)esters, (acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thioca.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In typical embodiments, the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis.

As used herein, the term “derivative” refers to a structurally similar compound that retains sufficient functional attributes of the identified analogue. The derivative may be structurally similar because it is lacking one or more atoms, substituted with one or more substituents, a salt, in different hydration/oxidation states, e.g., substituting a single or double bond, substituting a hydroxy group for a ketone, or because one or more atoms within the molecule are switched, such as, but not limited to, replacing an oxygen atom with a sulfur or nitrogen atom or replacing an amino group with a hydroxyl group or vice versa.

Replacing a carbon with nitrogen in an aromatic ring is a contemplated derivative. The derivative may be a prodrug. Derivatives may be prepared by any variety of synthetic methods or appropriate adaptations presented in the chemical literature or as in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

### Compounds

In certain embodiments, the disclosure relates to nucleosides conjugated to a phosphorus moiety or pharmaceutically acceptable salts thereof.

In certain embodiments, the disclosure relates to a compound of Formula I,

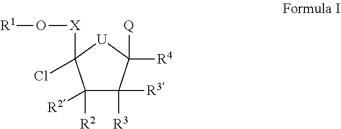

or a pharmaceutical or physiological salt thereof, wherein
X is CH₂, CHMe, CMe₂, CHF, CF₂, or CD₂;
U is O, S, NH, NR₇, CH₂, CHF, CF₂, CCH₂, or CCF₂;
Q is a natural or unnatural nucleobase;
R¹ is selected from H,

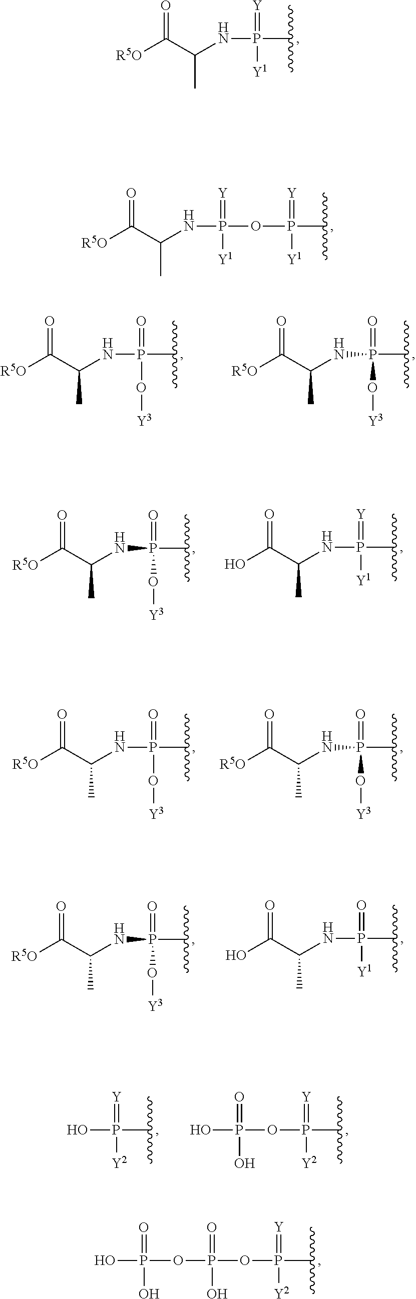

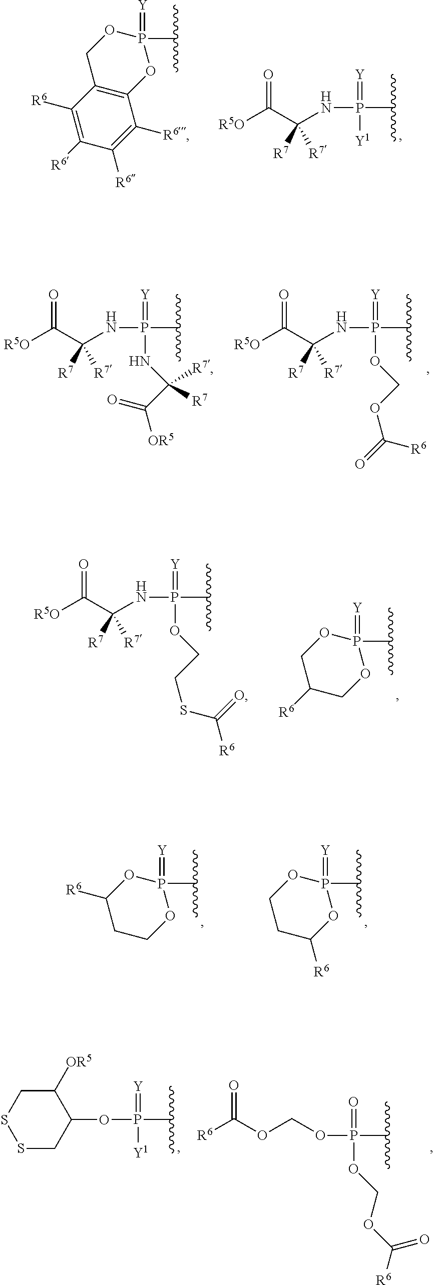

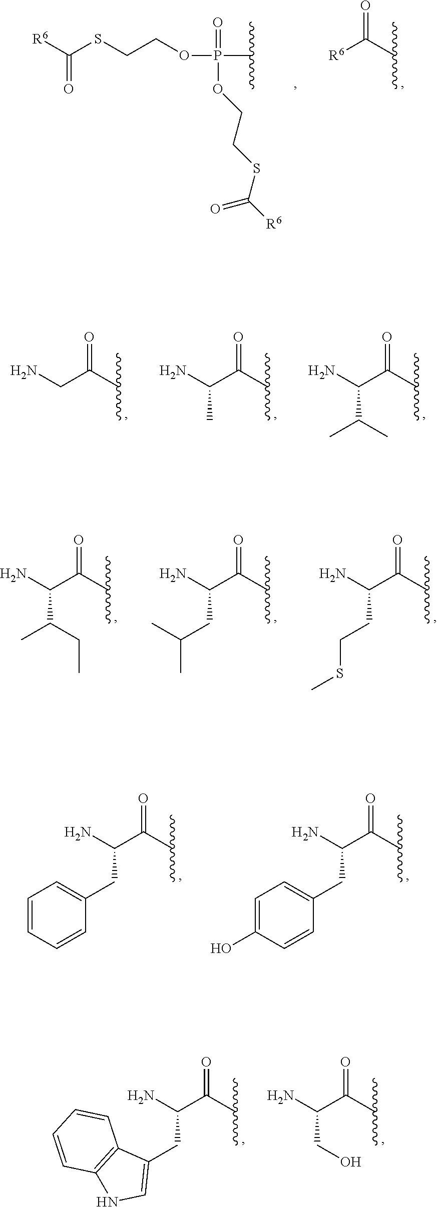

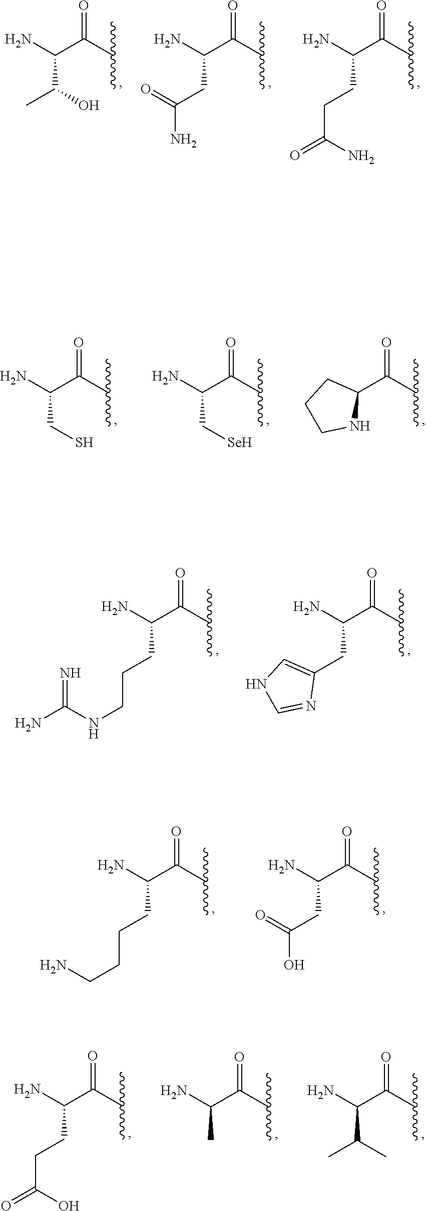

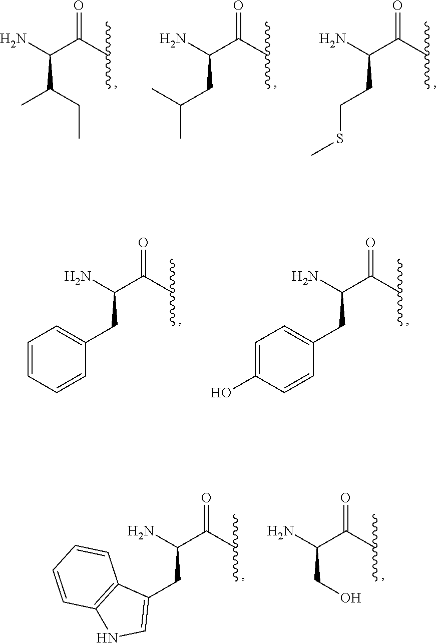

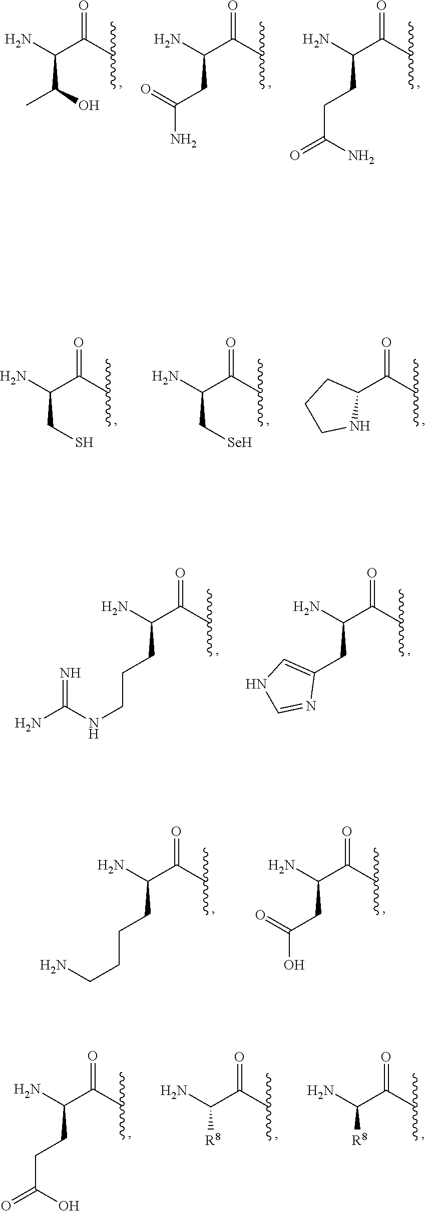

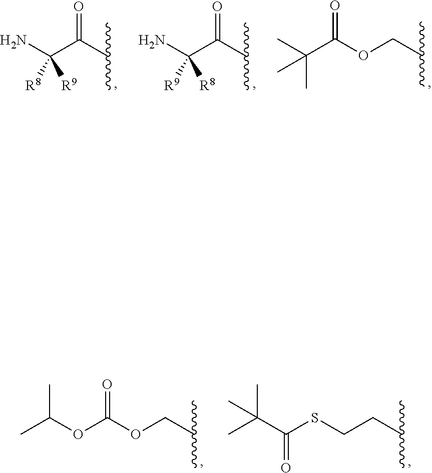

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R², R^{2′}, R³, R^{3′} are each independently selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R², R^{2′}, R³, R^{3′} are optionally substituted with one or more, the same or different, R¹⁰;
R² and R^{2′} can form a ring with the carbon they are attached to, wherein the ring is optionally substituted with one or more, the same or different, R¹⁰;
R³ and R^{3′} can form a ring with the carbon they are attached to, wherein the ring is optionally substituted with one or more, the same or different, R¹⁰;
R⁴ is hydrogen or deuterium;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6′}, and R^{6′″} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6′″} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In certain embodiments, the lipid is a fatty alcohol, fatty amine, or fatty thiol derived from essential and/or non-essential fatty acids.

In certain embodiments, the lipid is an unsaturated, polyunsaturated, omega unsaturated, or omega polyunsaturated fatty alcohol, fatty amine, or fatty thiol derived from essential and/or non-essential fatty acids.

In certain embodiments, the lipid is a fatty alcohol, fatty amine, or fatty thiol derived from essential and non-essential fatty acids that have one or more of its carbon units substituted with an oxygen, nitrogen, or sulfur.

In certain embodiments, the lipid is an unsaturated, polyunsaturated, omega unsaturated, or omega polyunsaturated fatty alcohol, fatty amine, or fatty thiol derived from essential and/or non-essential fatty acids that have one or more of its carbon units substituted with an oxygen, nitrogen, or sulfur.

In certain embodiments, the lipid is a fatty alcohol, fatty amine, or fatty thiol derived from essential and/or non-essential fatty acids that is optionally substituted.

In certain embodiments, the lipid is an unsaturated, polyunsaturated, omega unsaturated, or omega polyunsaturated fatty alcohol, fatty amine, or fatty thiol derived from essential and/or non-essential fatty acids that is optionally substituted.

In certain embodiments, the lipid is a fatty alcohol, fatty amine, or fatty thiol derived from essential and/or non-essential fatty acids that have one or more of its carbon units substituted with an oxygen, nitrogen, or sulfur that is optionally substituted.

In certain embodiments, the lipid is an unsaturated, polyunsaturated, omega unsaturated, or omega polyunsaturated fatty alcohol, fatty amine, or fatty thiol derived from essential and/or non-essential fatty acids that have one or more of its carbon units substituted with an oxygen, nitrogen, or sulfur that is also optionally substituted.

In certain embodiments, the lipid is hexadecyloxypropyl.

In certain embodiments, the lipid is 2-aminohexadecyloxypropyl.

In certain embodiments, the lipid is 2-aminoarachidyl.

In certain embodiments, the lipid is 2-benzyloxyhexadecyloxypropyl.

In certain embodiments, the lipid is lauryl, myristyl, palmityl, stearyl, arachidyl, behenyl, or lignoceryl.

In certain embodiments, the lipid is a sphingolipid of the formula:

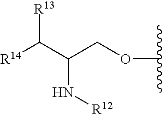

wherein,
R¹² of the sphingolipid is hydrogen, alkyl, C(═O)R¹⁶, C(═O)OR¹⁶, or C(═O)NHR¹⁶;
R¹³ of the sphingolipid is hydrogen, fluoro, OR¹⁶, OC(═O)R¹⁶, OC(═O)OR¹⁶, or OC(═O)NHR¹⁶;
R¹⁴ of the sphingolipid is a saturated or unsaturated alkyl chain of greater than 6 and less than 22 carbons optionally substituted with one or more halogen or hydroxy or a structure of the following formula:

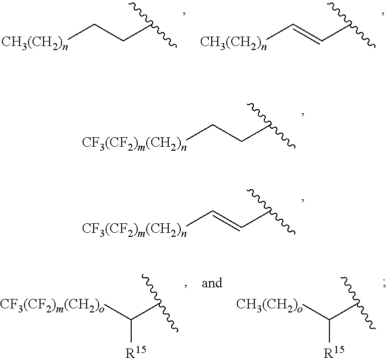

wherein n is 8 to 14 or less than or equal to 8 to less than or equal to 14, o is 9 to 15 or less than or equal to 9 to less than or equal to 15, the total or m and n is 8 to 14 or less than or equal to 8 to less than or equal to 14, the total of m and o is 9 to 15 or less than or equal to 9 to less than or equal to 15; or

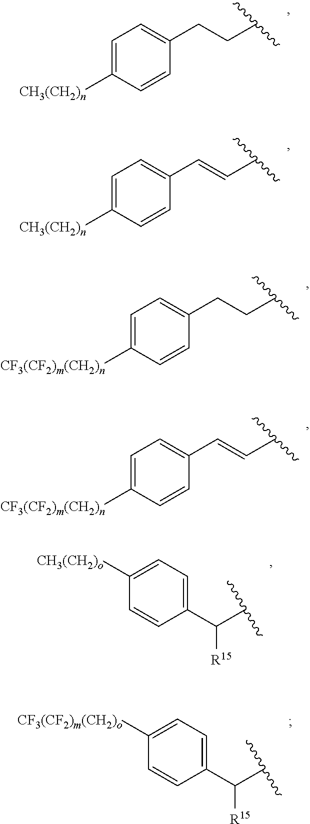

wherein n is 4 to 10 or less than or equal to 4 to less than or equal to 10, o is 5 to 11 or less than or equal to 5 to less than or equal to 11, the total of m and n is 4 to 10 or less than or equal to 4 to less than or equal to 10, and the total of m and o is 5 to 11 or less than or equal to 5 to less than or equal to 11; or

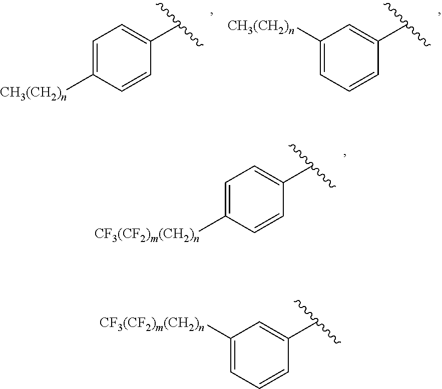

wherein n is 6 to 12 or n is less than or equal to 6 to less than or equal to 12, the total of m and n is 6 to 12 or n is less than or equal to 6 to less than or equal to 12;
R¹⁵ of the sphingolipid is OR¹⁶, OC(═O)R¹⁶, OC(═O)OR¹⁶, or OC(═O)NHR¹⁶;
R¹⁶ of the sphingolipid is hydrogen, cyano, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, or lipid; wherein R¹⁶ is optionally substituted with one or more, the same or different R¹⁷; and
R¹⁷ of the sphingolipid is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl.

In certain embodiments, R¹² of the sphingolipid is H, methyl, ethyl, propyl, n-butyl, isopropyl, 2-butyl, 1-ethylpropyl, 1-propylbutyl, cyclopropyl, cyclobutyl, cyclopentyl, cyclohexyl, benzyl, or phenyl.

In certain embodiments, the sphingolipid is a sphingolipid of the formula:

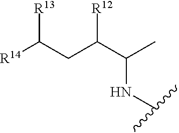

wherein,
R¹² of the sphingolipid is hydrogen, hydroxy, fluoro, OR¹⁶, OC(═O)R¹⁶, OC(═O)OR¹⁶, or OC(═O)NHR¹⁶;
R¹³ of the sphingolipid is hydrogen, hydroxy, fluoro, OR¹⁶, OC(═O)R¹⁶, OC(═O)OR¹⁶, or OC(═O)NHR¹⁶;
R¹⁴ of the sphingolipid is a saturated or unsaturated alkyl chain of greater than 6 and less than 22 carbons optionally substituted with one or more halogens or a structure of the following formula:

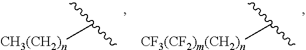

wherein n is 8 to 14 or less than or equal to 8 to less than or equal to 14, the total or m and n is 8 to 14 or less than or equal to 8 to less than or equal to 14;
R¹⁶ of the sphingolipid is hydrogen, cyano, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, or lipid; wherein R¹⁶ is optionally substituted with one or more, the same or different R¹⁷; and
R¹⁷ of the sphingolipid is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, esteryl, formyl, carboxy, carbamoyl, amido, or acyl.

In certain embodiments, R¹⁶ of the sphingolipid is H, methyl, ethyl, propyl, n-butyl, isopropyl, 2-butyl, 1-ethylpropyl, 1-propylbutyl, cyclopropyl, cyclobutyl, cyclopentyl, cyclohexyl, or benzyl.

Suitable sphingolipids include, but are not limited to, sphingosine, ceramide, or sphingomyelin, or 2-aminoalkyl optionally substituted with one or more substituents.

Other suitable sphingolipids include, but are not limited to, 2-aminooctadecane-3,5-diol; (2S, 3S, 5S)-2-aminooctadecane-3,5-diol; (2S, 3R, 5S)-2-aminooctadecane-3,5-diol; 2-(methylamino)octadecane-3,5-diol; (2S, 3R, 5S)-2-(methylamino)octadecane-3,5-diol; 2-(dimethylamino)octadecane-3,5-diol; (2R, 3S, 5S)-2-(dimethylamino)octadecane-3,5-diol; 1-(pyrrolidin-2-yl)hexadecane-1,3-diol; (1S, 3S)-1-((S)-pyrrolidin-2-yl)hexadecane-1,3-diol; 2-amino-11,11-difluorooctadecane-3,5-diol; (2S, 3S, 5S)-2-amino-11,11-difluorooctadecane-3,5-diol; 11,11-difluoro-2-(methylamino)octadecane-3,5-diol; (2S, 3S, 5S)-11,11-difluoro-2-(methylamino)octadecane-3,5-diol; N-((2S, 3S, 5S)-3,5-dihydroxyoctadecan-2-yl)acetamide; N-((2S, 3S, 5S)-3,5-dihydroxyoctadecan-2-yl)palmitamide; 1-(1-aminocyclopropyl)hexadecane-1,3-diol; (1S, 3R)-1-(1-aminocyclopropyl)hexadecane-1,3-diol; (1S, 3S)-1-(1-aminocyclopropyl)hexadecane-1,3-diol; 2-amino-2-methyloctadecane-3,5-diol; (3S, 5S)-2-amino-2-methyloctadecane-3,5-diol; (3S, 5R)-2-amino-2-methyloctadecane-3,5-diol; (3S, 5S)-2-methyl-2-(methylamino)octadecane-3,5-diol; 2-amino-5-hydroxy-2-methyloctadecan-3-one; (Z)-2-amino-5-hydroxy-2-methyloctadecan-3-one oxime; (2S, 3R, 5R)-2-amino-6,6-difluorooctadecane-3,5-diol; (2S, 3S, 5R)-2-amino-6,6-difluorooctadecane-3,5-diol; (2S, 3S, 5S)-2-amino-6,6-difluorooctadecane-3,5-diol; (2S, 3R, 5S)-2-amino-6,6-difluorooctadecane-3,5-diol; and (2S, 3S, 5S)-2-amino-18,18,18-trifluorooctadecane-3,5-diol, which can be optionally substituted with one or more substituents.

In exemplified embodiments of Formula I, R¹ is hydrogen,

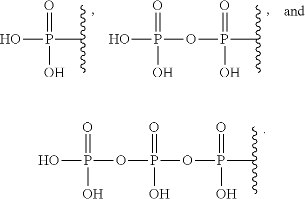

In exemplified embodiments of Formula I, X is CH₂.

In exemplified embodiments of Formula I, U is O.

In exemplified embodiments of Formula I, Q is uracil, cytosine, adenine, and guanine.

In exemplified embodiments of Formula I, R², R^{2′}, R³, R^{3′} are hydrogen, hydroxyl, amino, fluoro, chloro, cyano, methyl, fluoromethyl, methoxy, vinyl, ethynyl, and chloroethynyl.

In exemplified embodiments of Formula I, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula I, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula I, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula I, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula I, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula II,

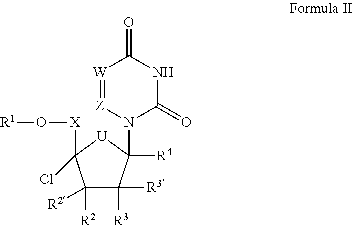

or a pharmaceutical or physiological salt thereof, wherein
X is CH₂, CHMe, CMe₂, CHF, CF₂, or CD₂;
U is O, S, NH, NR₇, CH₂, CHF, CF₂, CCH₂, or CCF₂;
W is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R″ is optionally substituted with one or more, the same or different, R¹⁰;
R¹ is selected from H,

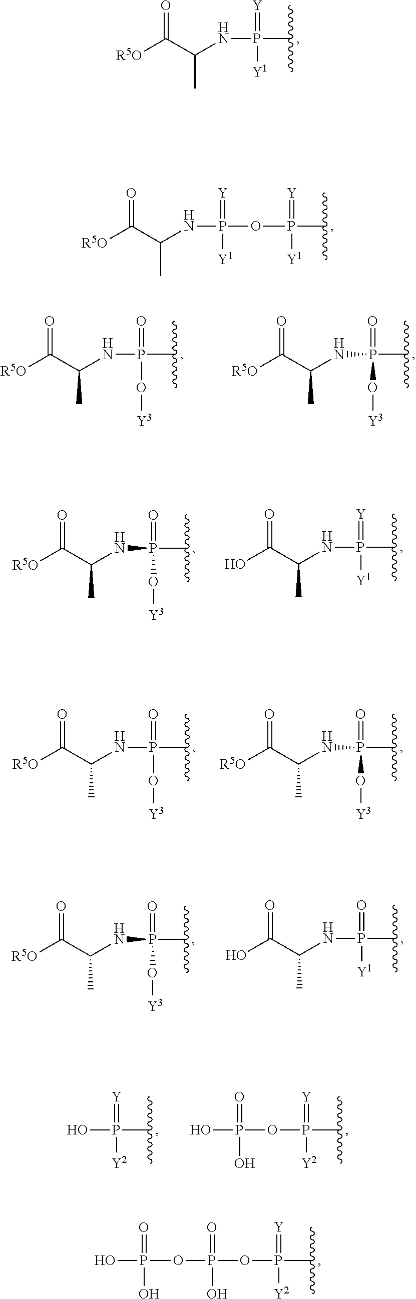

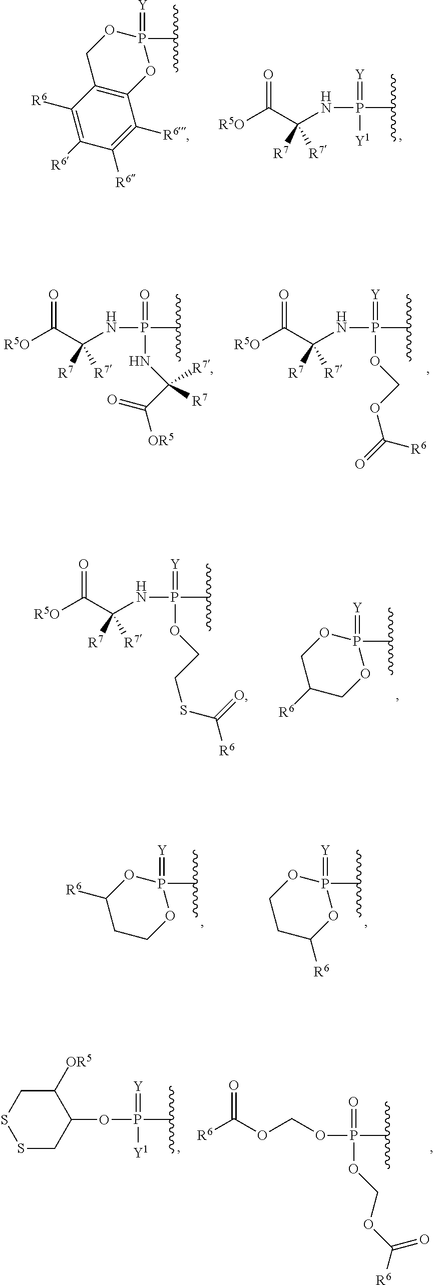

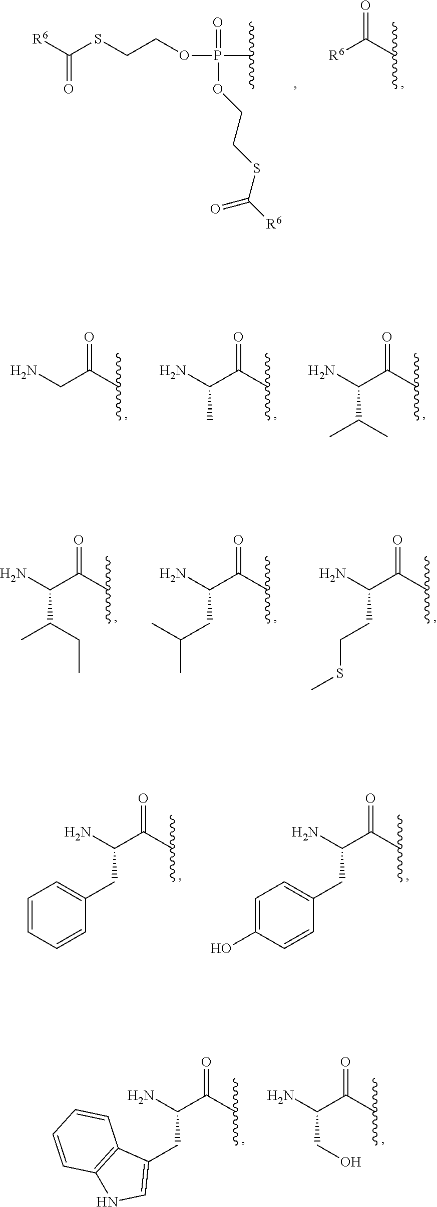

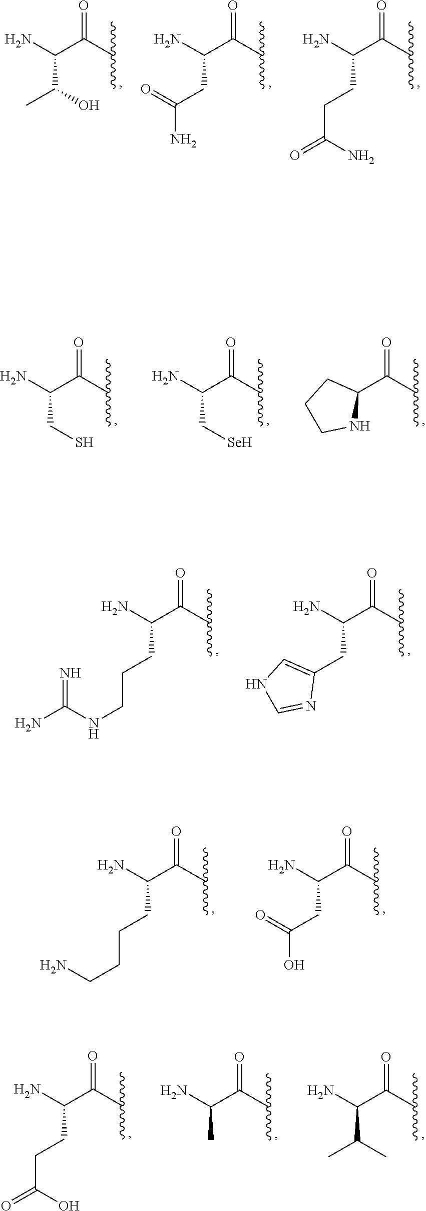

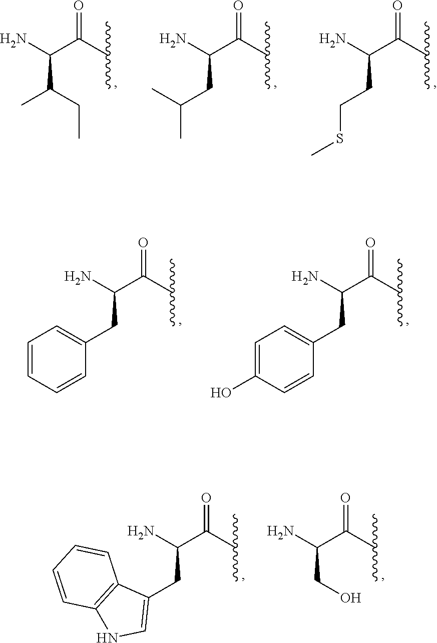

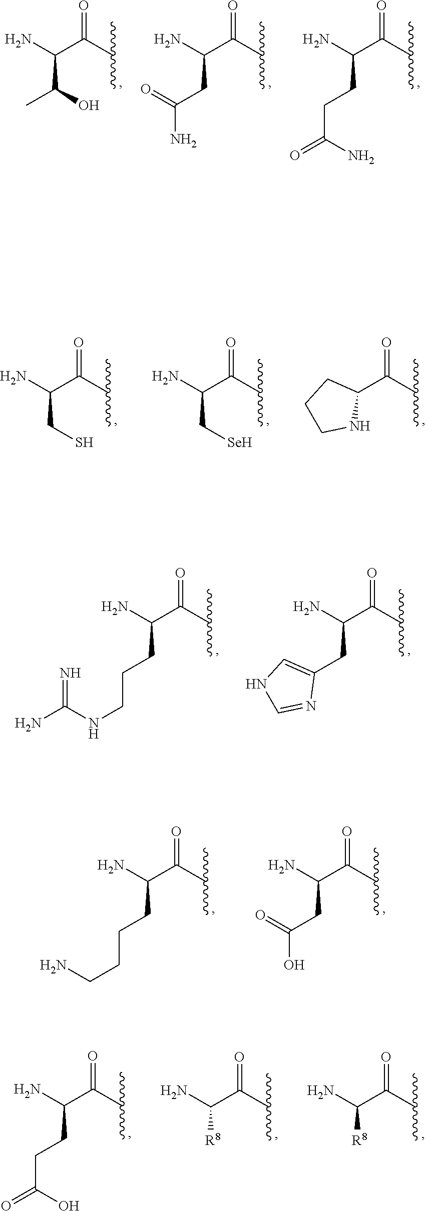

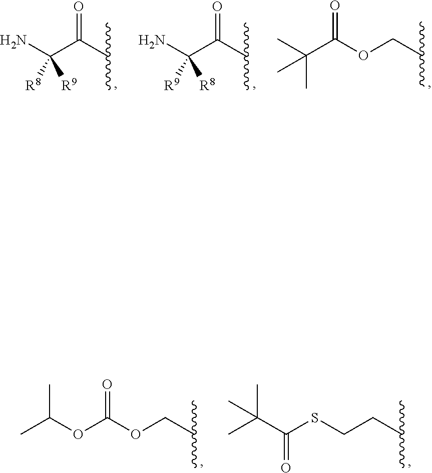

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R², R^{2′}, R³, R^{3′} are each independently selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R², R^{2′}, R³, R^{3′} are optionally substituted with one or more, the same or different, R¹⁰;
R² and R^{2′} can form a ring with the carbon they are attached to, wherein the ring is optionally substituted with one or more, the same or different, R¹⁰;
R³ and R^{3′} can form a ring with the carbon they are attached to, wherein the ring is optionally substituted with one or more, the same or different, R¹⁰;
R⁴ is hydrogen or deuterium;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6′″} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula II, R¹ is hydrogen,

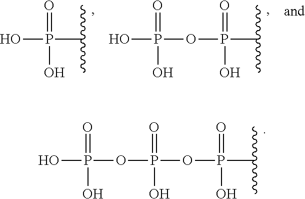

In exemplified embodiments of Formula II, X is CH₂.

In exemplified embodiments of Formula II, U is O.

In exemplified embodiments of Formula II, W is CR′.

In exemplified embodiments of Formula II, Z is CR″.

In exemplified embodiments of Formula II, R′ is H, F, Cl, OH, methyl, hydroxymethyl, fluoromethyl, difluoromethyl, trifluoromethyl, vinyl, ethynyl, formyl.

In exemplified embodiments of Formula II, R″ is H, F, Cl, OH, methyl, hydroxymethyl, fluoromethyl, difluoromethyl, trifluoromethyl, vinyl, ethynyl, formyl.

In exemplified embodiments of Formula II, R², R^{2′}, R³, R^{3′} are hydrogen, hydroxyl, amino, fluoro, chloro, cyano, methyl, fluoromethyl, methoxy, vinyl, ethynyl, and chloroethynyl.

In exemplified embodiments of Formula II, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula II, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula II, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula II, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula II, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula III,

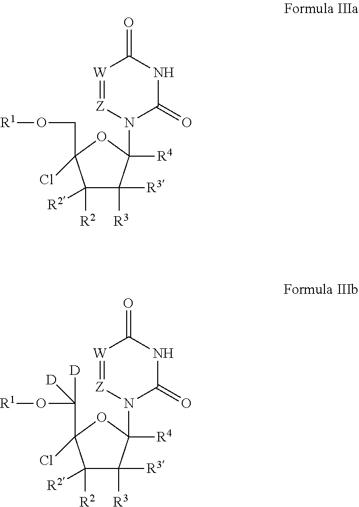

or a pharmaceutical or physiological salt thereof, wherein
W is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R″ is optionally substituted with one or more, the same or different, R¹⁰;
R¹ is selected from H,

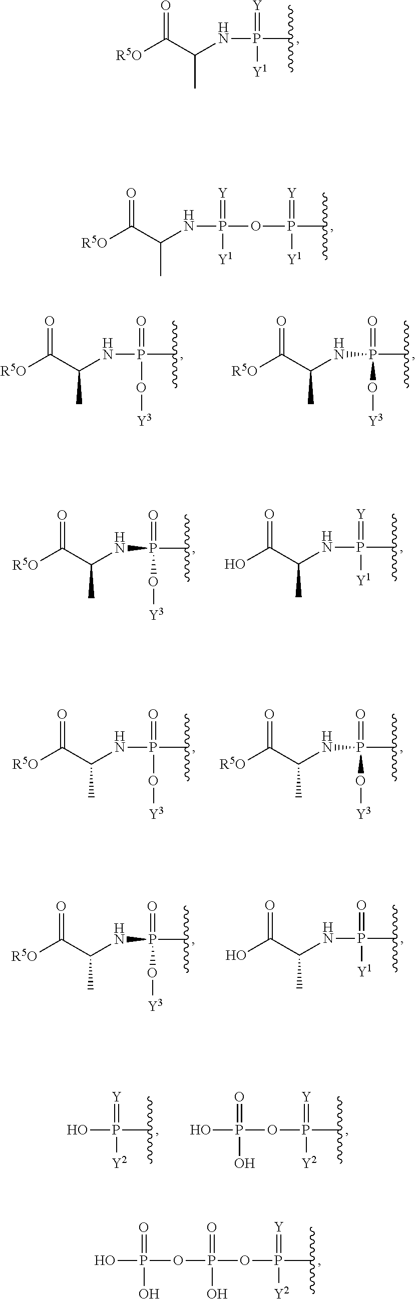

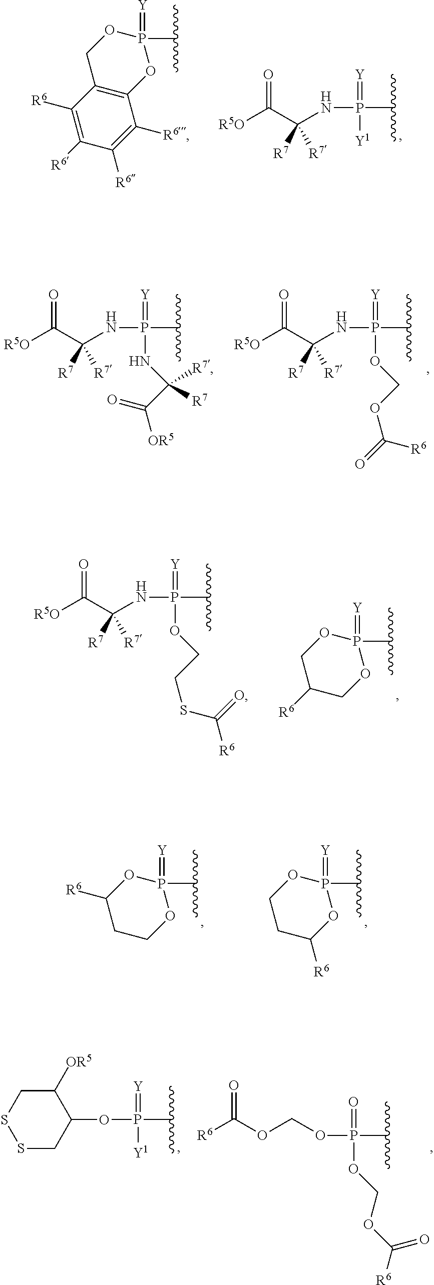

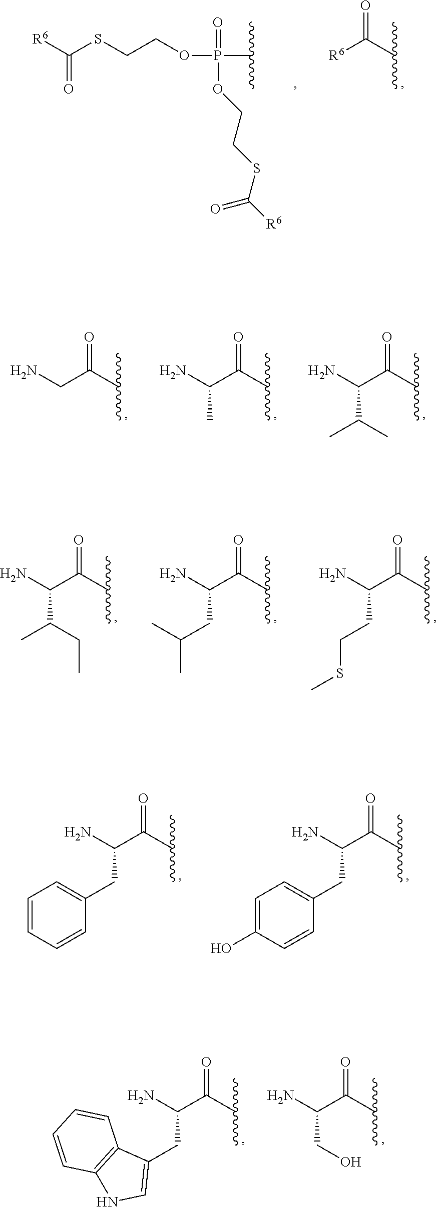

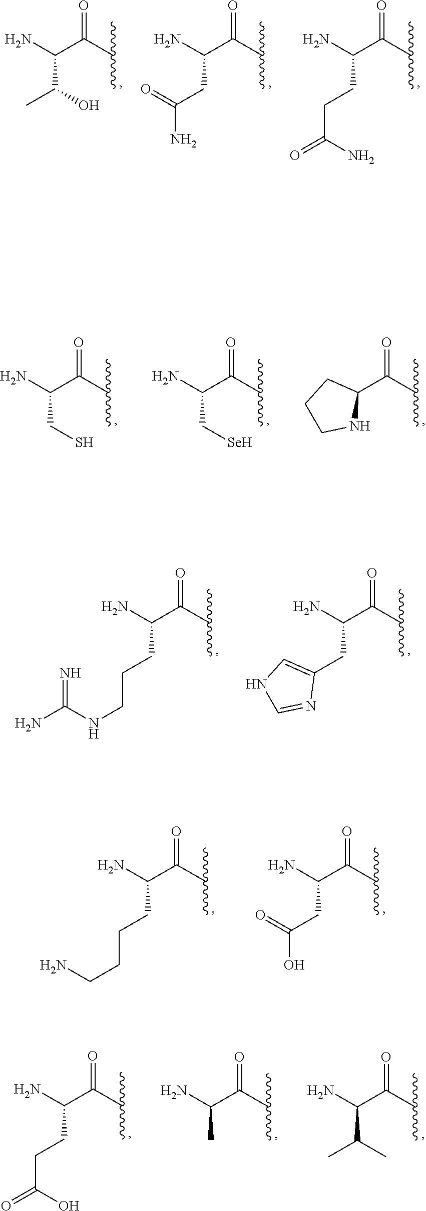

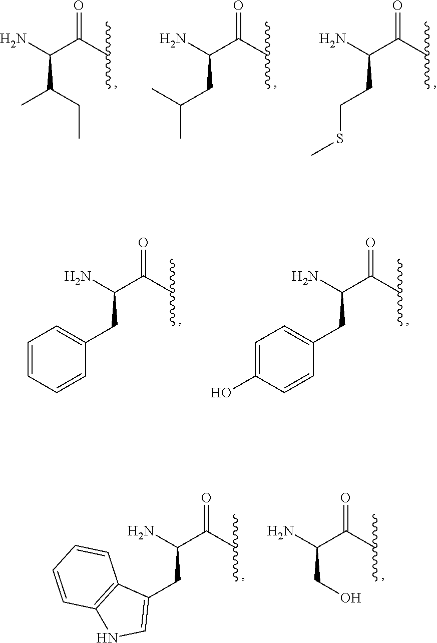

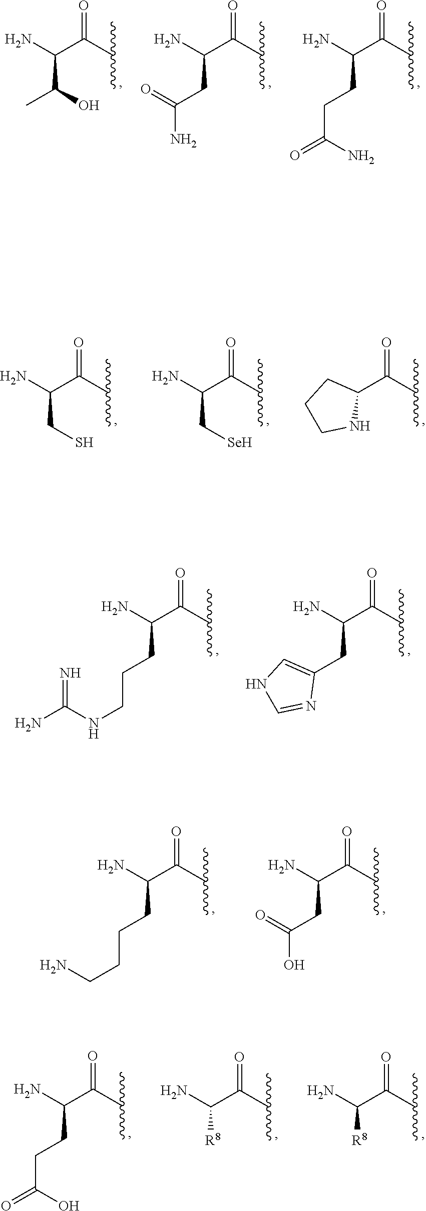

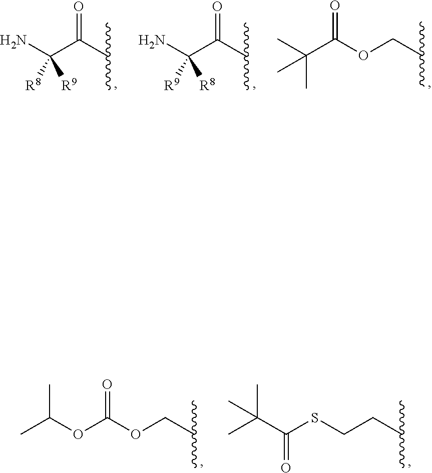

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R², R^{2′}, R³, R^{3′} are each independently selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R², R^{2′}, R³, R^{3′} are optionally substituted with one or more, the same or different, R¹⁰;
R² and R^{2′} can form a ring with the carbon they are attached to, wherein the ring is optionally substituted with one or more, the same or different, R¹⁰;
R³ and R^{3′} can form a ring with the carbon they are attached to, wherein the ring is optionally substituted with one or more, the same or different, R¹⁰;
R⁴ is hydrogen or deuterium;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula III, R¹ is hydrogen,

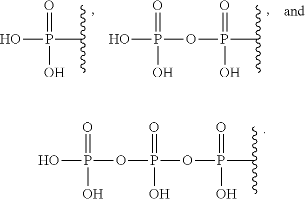

In exemplified embodiments of Formula III, W is CR′.

In exemplified embodiments of Formula III, Z is CR″.

In exemplified embodiments of Formula III, R′ is H, F, Cl, OH, methyl, hydroxymethyl, fluoromethyl, difluoromethyl, trifluoromethyl, vinyl, ethynyl, formyl.

In exemplified embodiments of Formula III, R″ is H, F, Cl, OH, methyl, hydroxymethyl, fluoromethyl, difluoromethyl, trifluoromethyl, vinyl, ethynyl, formyl.

In exemplified embodiments of Formula III, R², R^{2′}, R³, R^{3′} are hydrogen, hydroxyl, amino, fluoro, chloro, cyano, methyl, fluoromethyl, methoxy, vinyl, ethynyl, and chloroethynyl.

In exemplified embodiments of Formula III, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula III, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula III, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula III, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula III, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula IV,

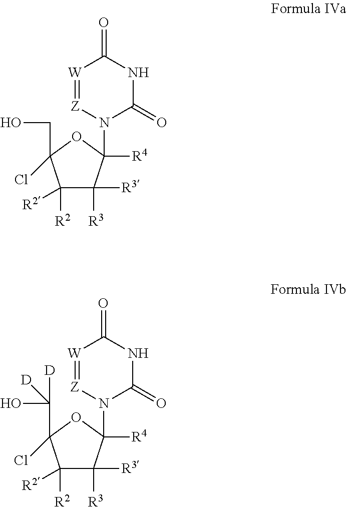

or a pharmaceutical or physiological salt thereof, wherein
W is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R″ is optionally substituted with one or more, the same or different, R¹⁰;
R², R^{2′}, R³, R^{3′} are each independently selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R², R^{2′}, R³, R^{3′} are optionally substituted with one or more, the same or different, R¹⁰;
R² and R^{2′} can form a ring with the carbon they are attached to, wherein the ring is optionally substituted with one or more, the same or different, R¹⁰;
R³ and R^{3′} can form a ring with the carbon they are attached to, wherein the ring is optionally substituted with one or more, the same or different, R¹⁰;
R⁴ is hydrogen or deuterium;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula IV, W is CR′.

In exemplified embodiments of Formula IV, Z is CR″.

In exemplified embodiments of Formula IV, R′ is H, F, Cl, OH, methyl, hydroxymethyl, fluoromethyl, difluoromethyl, trifluoromethyl, vinyl, ethynyl, formyl.

In exemplified embodiments of Formula IV, R″ is H, F, Cl, OH, methyl, hydroxymethyl, fluoromethyl, difluoromethyl, trifluoromethyl, vinyl, ethynyl, formyl.

In exemplified embodiments of Formula IV, R², R^{2′}, R³, R^{3′} are hydrogen, hydroxyl, amino, fluoro, chloro, cyano, methyl, fluoromethyl, methoxy, vinyl, ethynyl, and chloroethynyl.

In certain embodiments, the disclosure relates to a compound of Formula V,

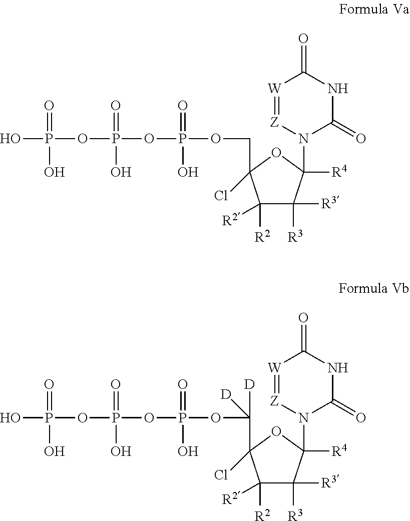

or a pharmaceutical or physiological salt thereof, wherein
W is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R″ is optionally substituted with one or more, the same or different, R¹⁰;
R², R^{2′}, R³, R^{3′} are each independently selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R², R^{2′}, R³, R^{3′} are optionally substituted with one or more, the same or different, R¹⁰;
R² and R^{2′} can form a ring with the carbon they are attached to, wherein the ring is optionally substituted with one or more, the same or different, R¹⁰;
R³ and R^{3′} can form a ring with the carbon they are attached to, wherein the ring is optionally substituted with one or more, the same or different, R¹⁰;
R⁴ is hydrogen or deuterium;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula V, W is CR′.

In exemplified embodiments of Formula V, Z is CR″.

In exemplified embodiments of Formula V, R′ is H, F, Cl, OH, methyl, hydroxymethyl, fluoromethyl, difluoromethyl, trifluoromethyl, vinyl, ethynyl, formyl.

In exemplified embodiments of Formula V, R″ is H, F, Cl, OH, methyl, hydroxymethyl, fluoromethyl, difluoromethyl, trifluoromethyl, vinyl, ethynyl, formyl.

In exemplified embodiments of Formula V, R², R^{2′}, R³, R^{3′} are hydrogen, hydroxyl, amino, fluoro, chloro, cyano, methyl, fluoromethyl, methoxy, vinyl, ethynyl, and chloroethynyl.

In certain embodiments, the disclosure relates to a compound of Formula VI,

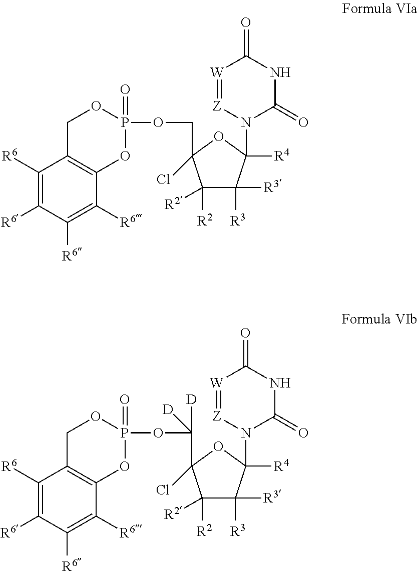

or a pharmaceutical or physiological salt thereof, wherein
W is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R″ is optionally substituted with one or more, the same or different, R¹⁰;
R², R^{2′}, R³, R^{3′} are each independently selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R², R^{2′}, R³, R^{3′} are optionally substituted with one or more, the same or different, R¹⁰;
R² and R^{2′} can form a ring with the carbon they are attached to, wherein the ring is optionally substituted with one or more, the same or different, R¹⁰;
R³ and R^{3′} can form a ring with the carbon they are attached to, wherein the ring is optionally substituted with one or more, the same or different, R¹⁰;
R⁴ is hydrogen or deuterium;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula VI, W is CR′.

In exemplified embodiments of Formula VI, Z is CR″.

In exemplified embodiments of Formula VI, R′ is H, F, Cl, OH, methyl, hydroxymethyl, fluoromethyl, difluoromethyl, trifluoromethyl, vinyl, ethynyl, formyl.

In exemplified embodiments of Formula VI, R″ is H, F, Cl, OH, methyl, hydroxymethyl, fluoromethyl, difluoromethyl, trifluoromethyl, vinyl, ethynyl, formyl.

In exemplified embodiments of Formula VI, R², R^{2′}, R³, R^{3′} are hydrogen, hydroxyl, amino, fluoro, chloro, cyano, methyl, fluoromethyl, methoxy, vinyl, ethynyl, and chloroethynyl.

In exemplified embodiments of Formula VI, R⁶, R^{6′}, R^{6″}, and R^{6″′} are all hydrogen.

In exemplified embodiments of Formula VI, R⁶, R^{6′}, and R^{6″} are hydrogen and R^{6′} is methyl.

In exemplified embodiments of Formula VI, R⁶, R^{6′}, and R^{6″} are hydrogen and R^{6′} is methoxy.

In exemplified embodiments of Formula VI, R⁶, R^{6″}, and R^{6′} are hydrogen and R^{6′} is methyl.

In exemplified embodiments of Formula VI, R⁶, R^{6″}, and R^{6′″} are hydrogen and R^{6′} is methoxy.

In exemplified embodiments of Formula VI, R⁶, R^{6″}, and R^{6′} are hydrogen and R^{6′} is fluoro.

In exemplified embodiments of Formula VI, R⁶, R^{6″}, and R^{6′} are hydrogen and R^{6′} I tert-butyl.

In exemplified embodiments of Formula VI, R⁶, R^{6″}, and R^{6′} are hydrogen and R^{6′} is chloro.

In exemplified embodiments of Formula VI, R^{6′} and R^{6″} are hydrogen and R^{6′} and R^{6′″} are methyl.

In exemplified embodiments of Formula VI, R^{6′}, R^{6″}, and R^{6″′} are hydrogen and R⁶ is fluoro.

In exemplified embodiments of Formula VI, R^{6″} is hydrogen, R^{6′} and R^{6′″} are tert-butyl, and R⁶ is fluoro.

In certain embodiments, the disclosure relates to a compound of Formula VII,

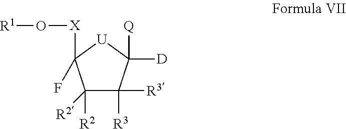

or a pharmaceutical or physiological salt thereof, wherein
X is CH₂, CHMe, CMe₂, CHF, CF₂, or CD₂;
U is O, S, NH, NR₇, CH₂, CHF, CF₂, CCH₂, or CCF₂;
Q is a natural or unnatural nucleobase;
R¹ is selected from H,

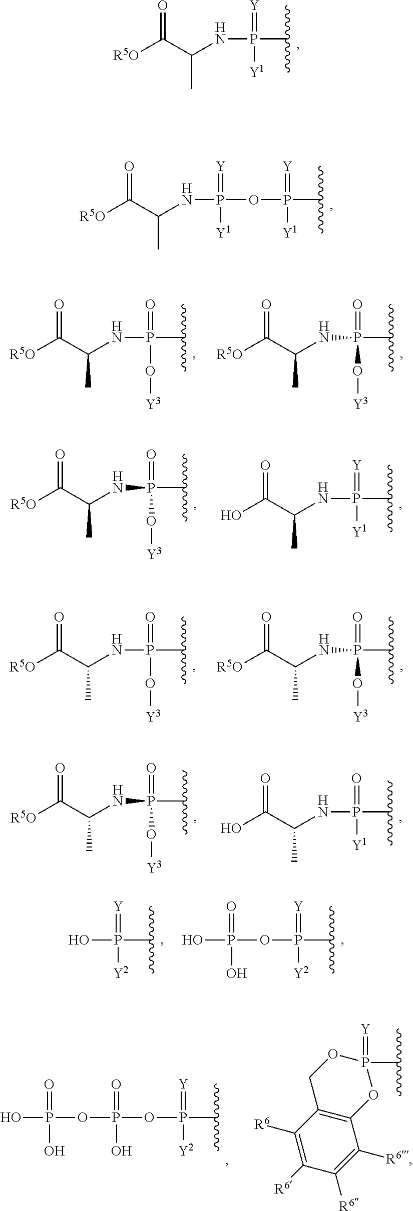

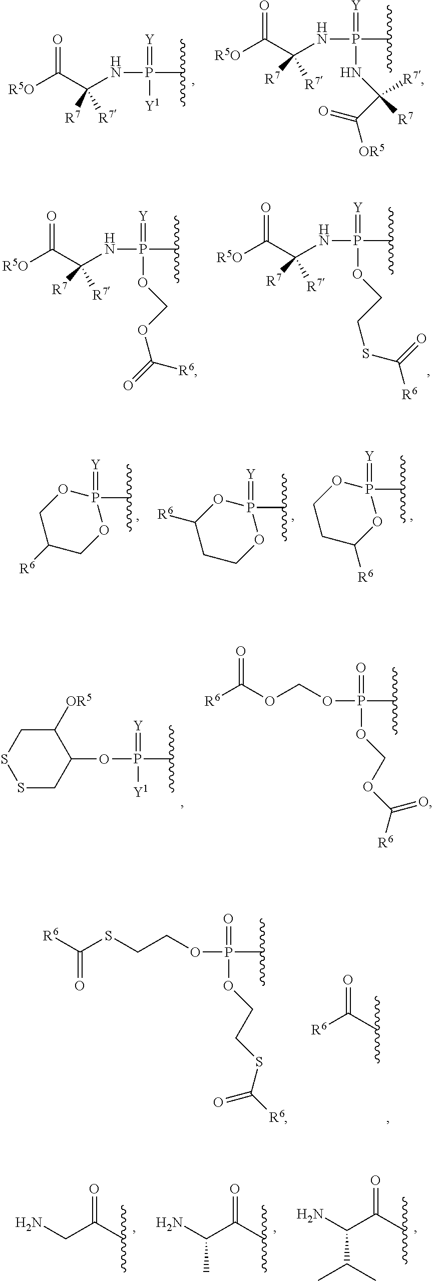

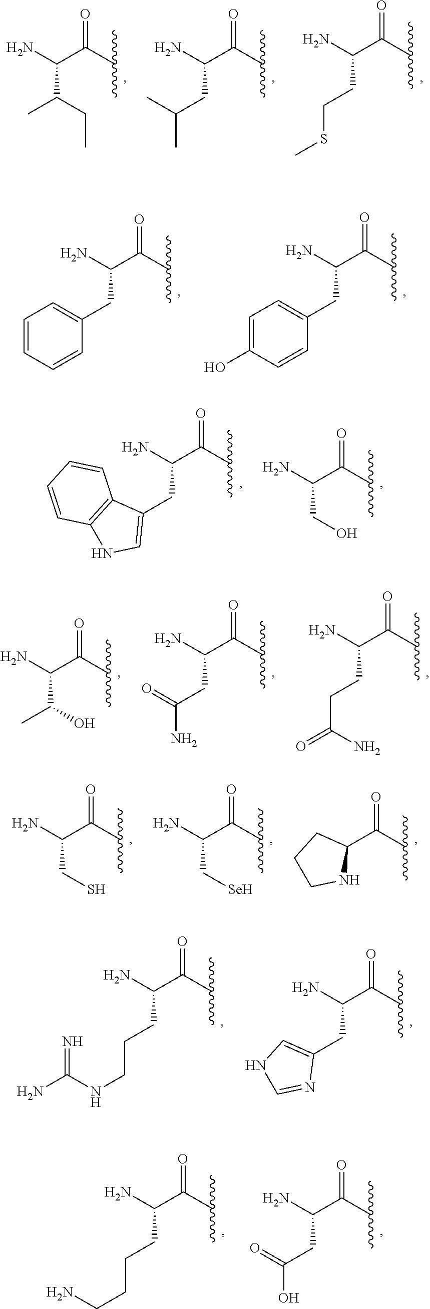

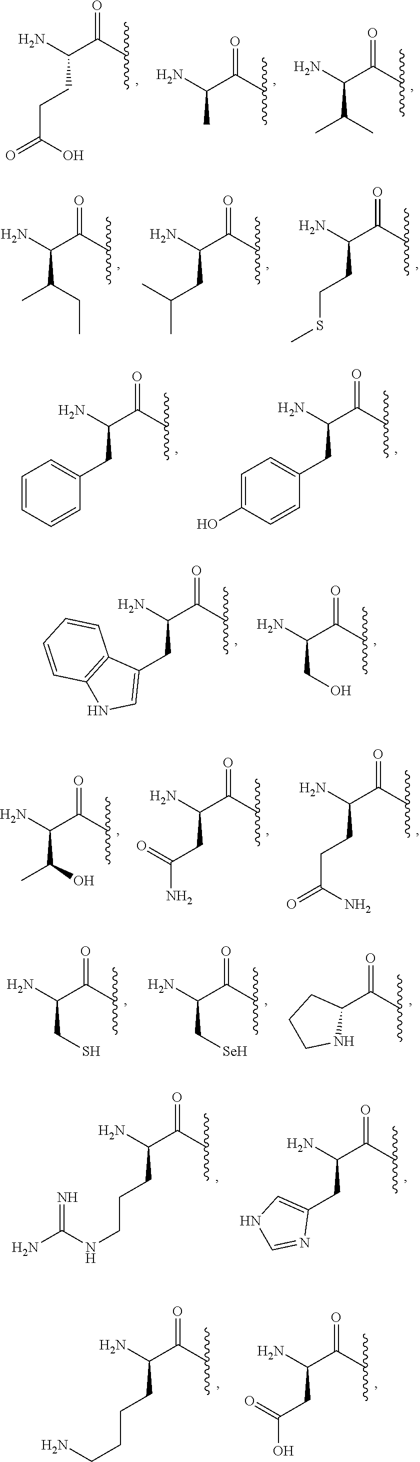

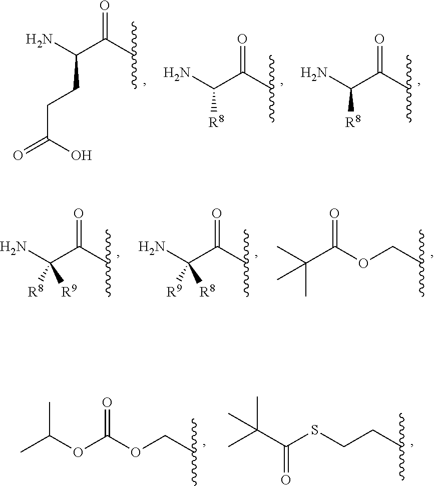

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R², R^{2′}, R³, R^{3′} are each independently selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R², R^{2′}, R³, R^{3′} are optionally substituted with one or more, the same or different, R¹⁰;
R² and R^{2′} can form a ring with the carbon they are attached to, wherein the ring is optionally substituted with one or more, the same or different, R¹⁰;
R³ and R^{3′} can form a ring with the carbon they are attached to, wherein the ring is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and

Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula VII, R¹ is hydrogen,

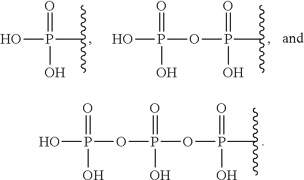

In exemplified embodiments of Formula VII, X is CH₂.

In exemplified embodiments of Formula VII, U is O.

In exemplified embodiments of Formula VII, Q is uracil, cytosine, adenine, and guanine.

In exemplified embodiments of Formula VII, R², R^{2′}, R³, R^{3′} are hydrogen, hydroxyl, amino, fluoro, chloro, cyano, methyl, fluoromethyl, methoxy, vinyl, ethynyl, and chloroethynyl.

In exemplified embodiments of Formula VII, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula VII, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula VII, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula VII, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula VII, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula VIII,

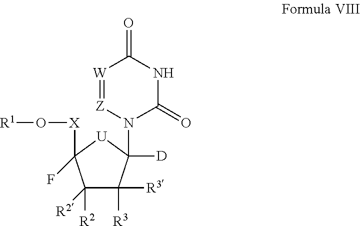

or a pharmaceutical or physiological salt thereof, wherein
X is CH₂, CHMe, CMe₂, CHF, CF₂, or CD₂;
U is O, S, NH, NR₇, CH₂, CHF, CF₂, CCH₂, or CCF₂;
W is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R″ is optionally substituted with one or more, the same or different, R¹⁰;
R¹ is selected from H,

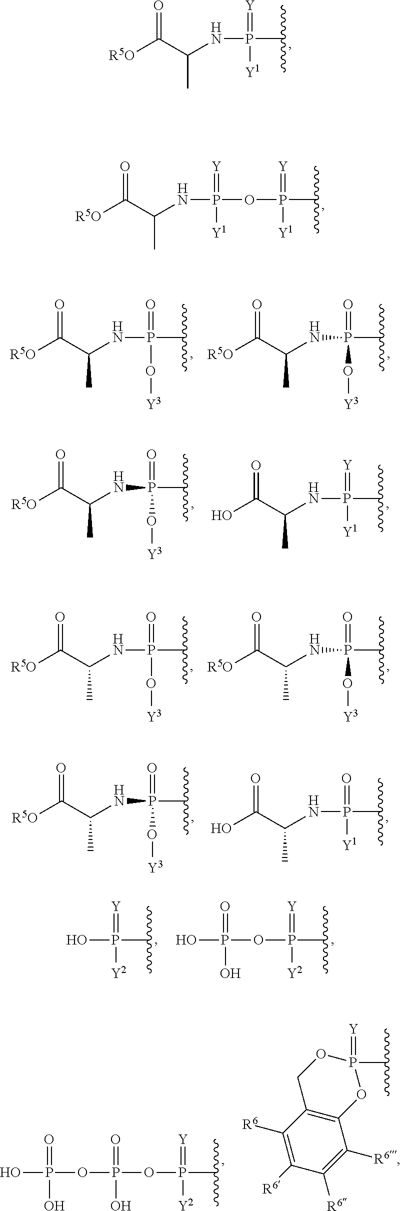

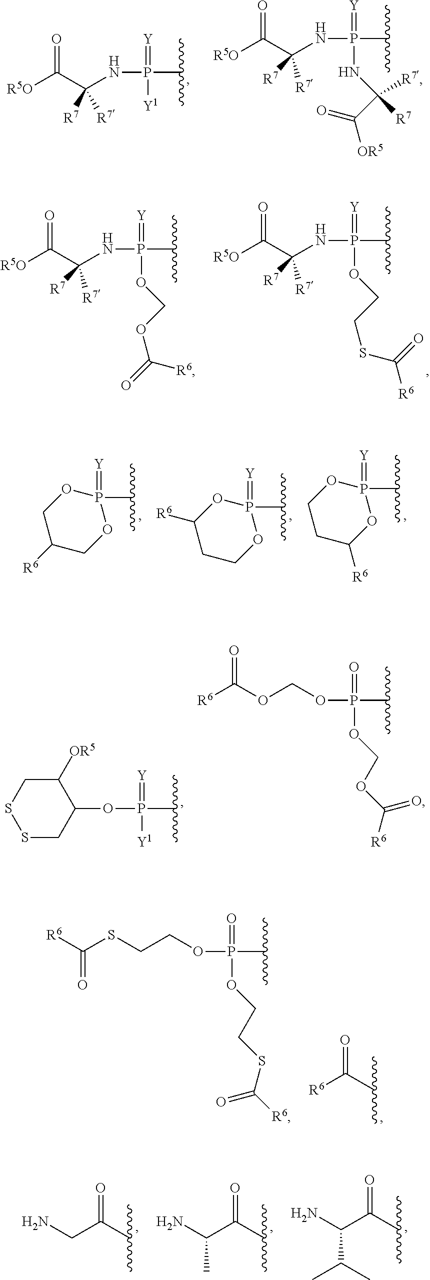

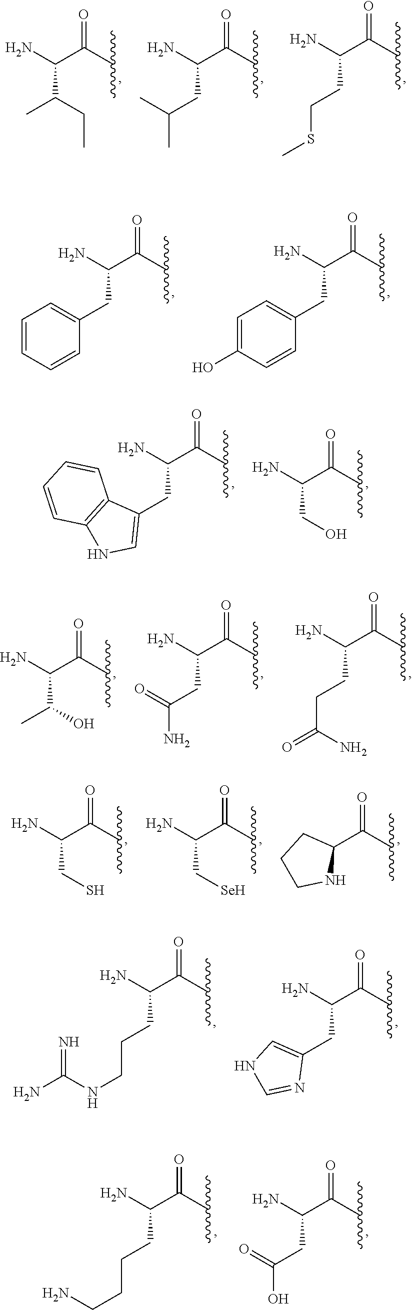

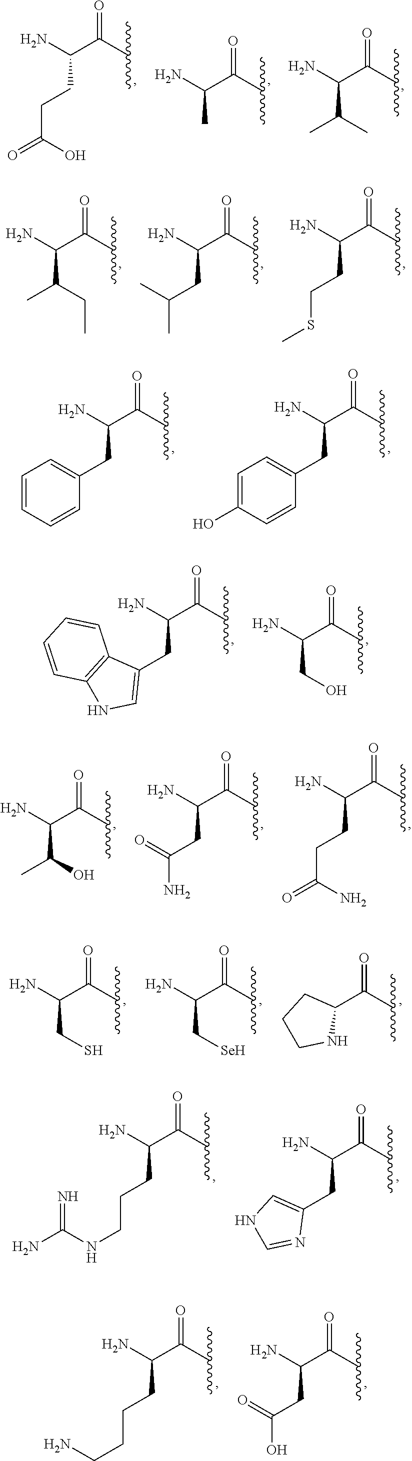

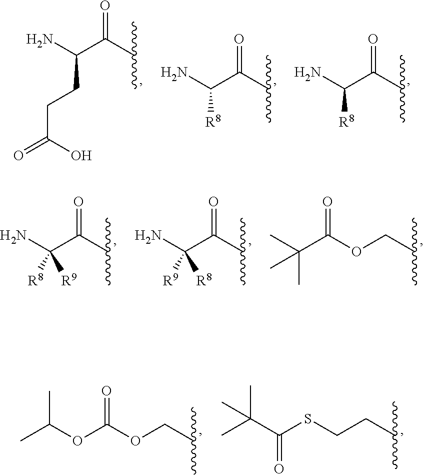

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R², R^{2′}, R³, R^{3′} are each independently selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R², R^{2′}, R³, R^{3′} are optionally substituted with one or more, the same or different, R¹⁰;
R² and R^{2′} can form a ring with the carbon they are attached to, wherein the ring is optionally substituted with one or more, the same or different, R¹⁰;
R³ and R^{3′} can form a ring with the carbon they are attached to, wherein the ring is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula VIII, R¹ is hydrogen, OH

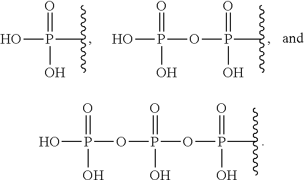

In exemplified embodiments of Formula VIII, X is CH₂.

In exemplified embodiments of Formula VIII, U is O.

In exemplified embodiments of Formula VIII, W is CR′.

In exemplified embodiments of Formula VIII, Z is CR″.

In exemplified embodiments of Formula VIII, R′ is H, F, Cl, OH, methyl, hydroxymethyl, fluoromethyl, difluoromethyl, trifluoromethyl, vinyl, ethynyl, formyl.

In exemplified embodiments of Formula VIII, R″ is H, F, Cl, OH, methyl, hydroxymethyl, fluoromethyl, difluoromethyl, trifluoromethyl, vinyl, ethynyl, formyl.

In exemplified embodiments of Formula VIII, R², R^{2′}, R³, R^{3′} are hydrogen, hydroxyl, amino, fluoro, chloro, cyano, methyl, fluoromethyl, methoxy, vinyl, ethynyl, and chloroethynyl.

In exemplified embodiments of Formula VIII, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula VIII, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula VIII, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula VIII, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula VIII, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula IX,

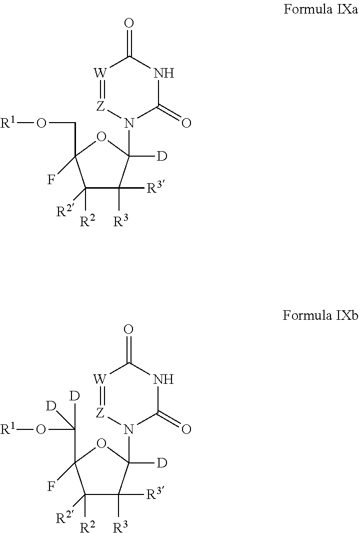

or a pharmaceutical or physiological salt thereof, wherein
W is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R″ is optionally substituted with one or more, the same or different, R¹⁰;
R¹ is selected from H,

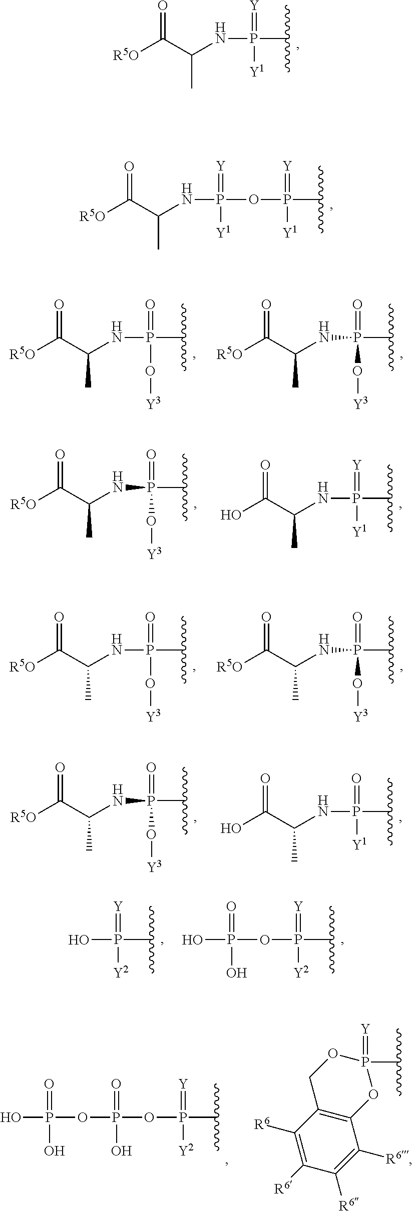

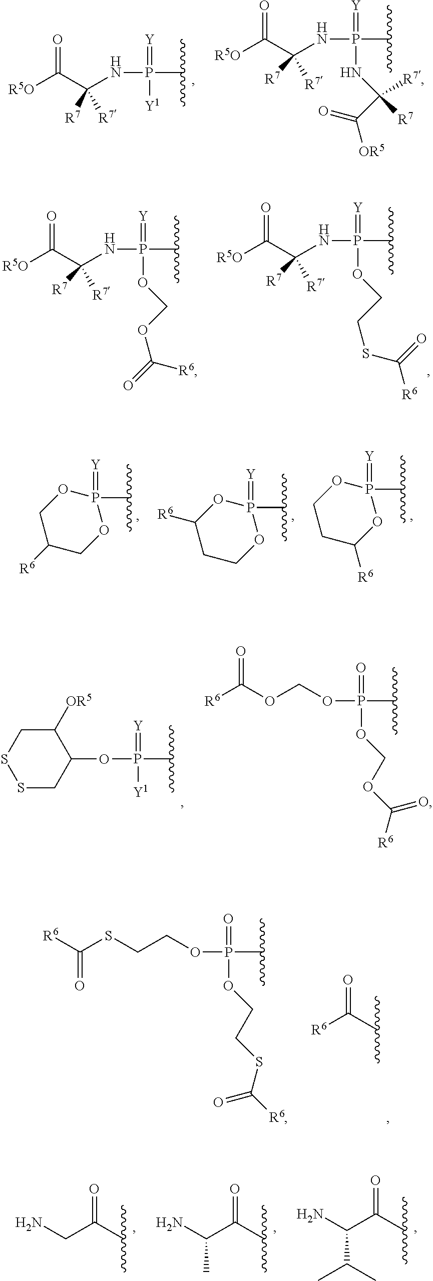

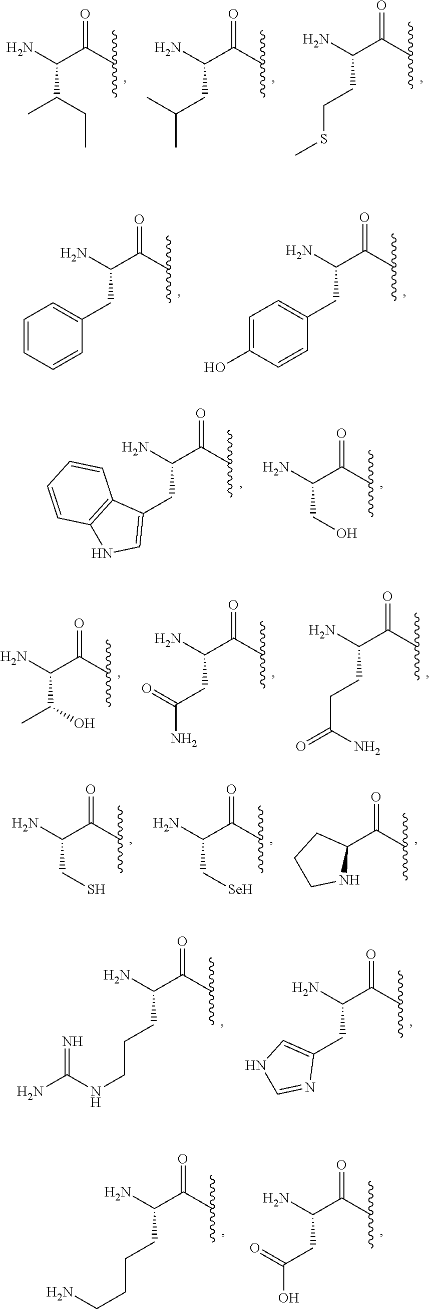

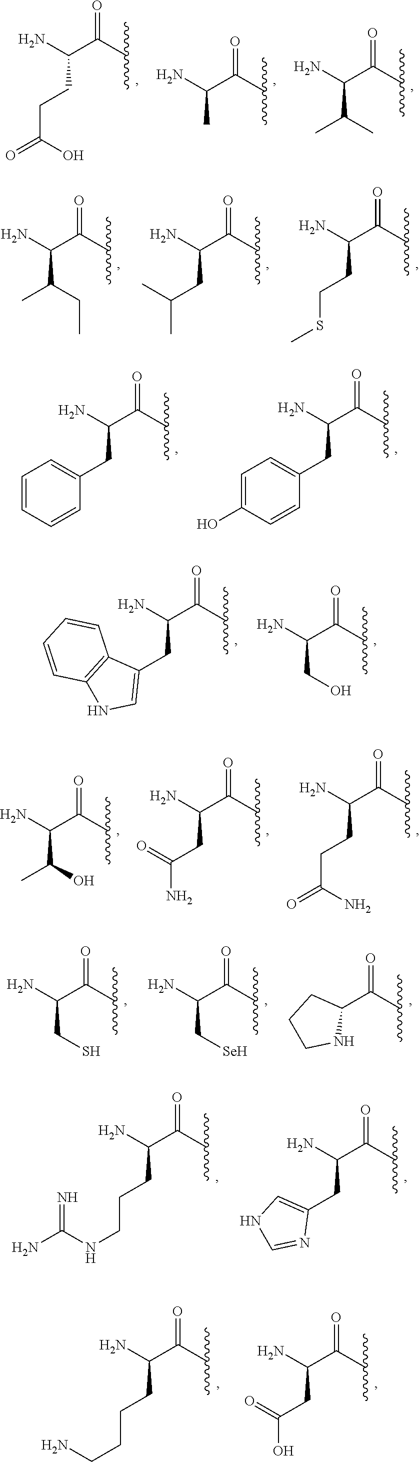

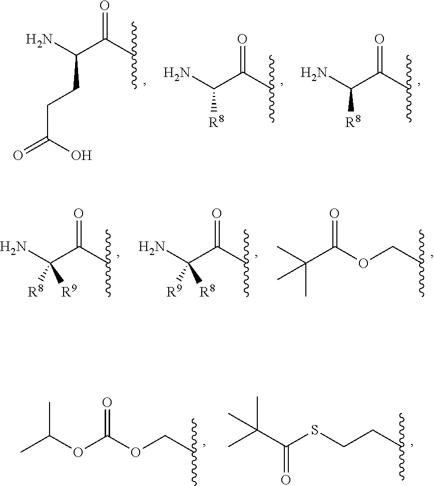

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R², R^{2′}, R³, R^{3′} are each independently selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R², R^{2′}, R³, R^{3′} are optionally substituted with one or more, the same or different, R¹⁰;
R² and R^{2′} can form a ring with the carbon they are attached to, wherein the ring is optionally substituted with one or more, the same or different, R¹⁰;
R³ and R^{3′} can form a ring with the carbon they are attached to, wherein the ring is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula IX, R¹ is hydrogen,

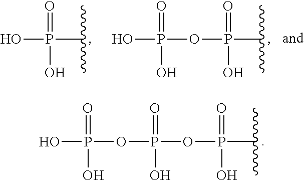

In exemplified embodiments of Formula IX, W is CR′.

In exemplified embodiments of Formula IX, Z is CR″.

In exemplified embodiments of Formula IX, R′ is H, F, Cl, OH, methyl, hydroxymethyl, fluoromethyl, difluoromethyl, trifluoromethyl, vinyl, ethynyl, formyl.

In exemplified embodiments of Formula IX, R″ is H, F, Cl, OH, methyl, hydroxymethyl, fluoromethyl, difluoromethyl, trifluoromethyl, vinyl, ethynyl, formyl.

In exemplified embodiments of Formula IX, R², R^{2′}, R³, R^{3′} are hydrogen, hydroxyl, amino, fluoro, chloro, cyano, methyl, fluoromethyl, methoxy, vinyl, ethynyl, and chloroethynyl.

In exemplified embodiments of Formula IX, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula IX, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula IX, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula IX, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula IX, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula X,

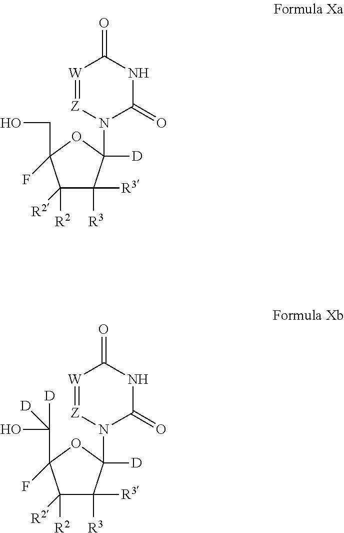

or a pharmaceutical or physiological salt thereof, wherein
W is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R″ is optionally substituted with one or more, the same or different, R¹⁰;
R², R^{2′}, R³, R^{3′} are each independently selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R², R^{2′}, R³, R^{3′} are optionally substituted with one or more, the same or different, R¹⁰;
R² and R^{2′} can form a ring with the carbon they are attached to, wherein the ring is optionally substituted with one or more, the same or different, R¹⁰;
R³ and R^{3′} can form a ring with the carbon they are attached to, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula X, W is CR′.

In exemplified embodiments of Formula X, Z is CR″.

In exemplified embodiments of Formula X, R′ is H, F, Cl, OH, methyl, hydroxymethyl, fluoromethyl, difluoromethyl, trifluoromethyl, vinyl, ethynyl, formyl.

In exemplified embodiments of Formula X, R″ is H, F, Cl, OH, methyl, hydroxymethyl, fluoromethyl, difluoromethyl, trifluoromethyl, vinyl, ethynyl, formyl.

In exemplified embodiments of Formula X, R², R^{2′}, R³, R^{3′} are hydrogen, hydroxyl, amino, fluoro, chloro, cyano, methyl, fluoromethyl, methoxy, vinyl, ethynyl, and chloroethynyl.

In certain embodiments, the disclosure relates to a compound of Formula XI,

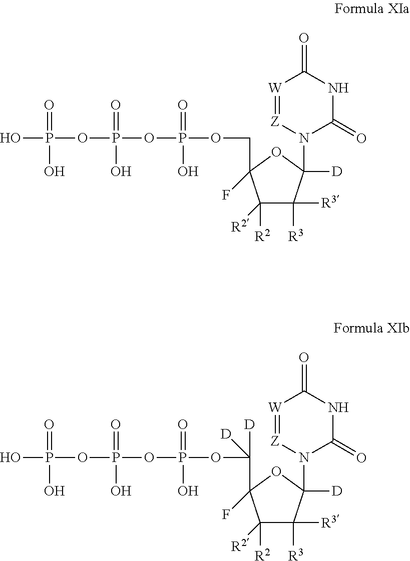

or a pharmaceutical or physiological salt thereof, wherein
W is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R″ is optionally substituted with one or more, the same or different, R¹⁰;
R², R^{2′}, R³, R^{3′} are each independently selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R², R^{2′}, R³, R^{3′} are optionally substituted with one or more, the same or different, R¹⁰;
R² and R^{2′} can form a ring with the carbon they are attached to, wherein the ring is optionally substituted with one or more, the same or different, R¹⁰;
R³ and R^{3′} can form a ring with the carbon they are attached to, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R″ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula XI, W is CR′.

In exemplified embodiments of Formula XI, Z is CR″.

In exemplified embodiments of Formula XI, R′ is H, F, Cl, OH, methyl, hydroxymethyl, fluoromethyl, difluoromethyl, trifluoromethyl, vinyl, ethynyl, formyl.

In exemplified embodiments of Formula XI, R″ is H, F, Cl, OH, methyl, hydroxymethyl, fluoromethyl, difluoromethyl, trifluoromethyl, vinyl, ethynyl, formyl.

In exemplified embodiments of Formula XI, R², R^{2′}, R³, R^{3′} are hydrogen, hydroxyl, amino, fluoro, chloro, cyano, methyl, fluoromethyl, methoxy, vinyl, ethynyl, and chloroethynyl.

In certain embodiments, the disclosure relates to a compound of Formula XII,

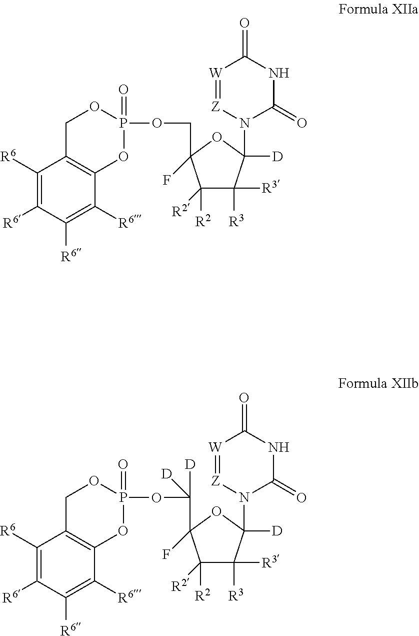

or a pharmaceutical or physiological salt thereof, wherein
W is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R″ is optionally substituted with one or more, the same or different, R¹⁰;
R², R^{2′}, R³, R^{3′} are each independently selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R², R^{2′}, R³, R^{3′} are optionally substituted with one or more, the same or different, R¹⁰;
R² and R^{2′} can form a ring with the carbon they are attached to, wherein the ring is optionally substituted with one or more, the same or different, R¹⁰;
R³ and R^{3′} can form a ring with the carbon they are attached to, wherein the ring is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R″ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula XII, W is CR′.

In exemplified embodiments of Formula XII, Z is CR″.

In exemplified embodiments of Formula XII, R′ is H, F, Cl, OH, methyl, hydroxymethyl, fluoromethyl, difluoromethyl, trifluoromethyl, vinyl, ethynyl, formyl.

In exemplified embodiments of Formula XII, R″ is H, F, Cl, OH, methyl, hydroxymethyl, fluoromethyl, difluoromethyl, trifluoromethyl, vinyl, ethynyl, formyl.

In exemplified embodiments of Formula XII, R², R^{2′}, R³, R^{3′} are hydrogen, hydroxyl, amino, fluoro, chloro, cyano, methyl, fluoromethyl, methoxy, vinyl, ethynyl, and chloroethynyl.

In exemplified embodiments of Formula XII, R⁶, R^{6′}, R^{6″}, and R^{6″′} are all hydrogen.

In exemplified embodiments of Formula XII, R⁶, R^{6′}, and R^{6″} are hydrogen and R^{6″′} is methyl.

In exemplified embodiments of Formula XII, R⁶, R^{6′}, and R^{6″} are hydrogen and R^{6″′} is methoxy.

In exemplified embodiments of Formula XII, R⁶, R^{6″}, and R^{6″′} are hydrogen and R^{6′} is methyl.

In exemplified embodiments of Formula XII, R⁶, R^{6″}, and R^{6″′} are hydrogen and R^{6′} is methoxy.

In exemplified embodiments of Formula XII, R⁶, R^{6″}, and R^{6″′} are hydrogen and R^{6′} is fluoro.

In exemplified embodiments of Formula XII, R⁶, R^{6″}, and R^{6″′} are hydrogen and R^{6′} I tert-butyl.

In exemplified embodiments of Formula XII, R⁶, R^{6″}, and R^{6″′} are hydrogen and R^{6′} is chloro.

In exemplified embodiments of Formula XII, R⁶ and R^{6″} are hydrogen and R^{6′} and R″′ are methyl.

In exemplified embodiments of Formula XII, R^{6′}, R^{6″}, and R^{6′} are hydrogen and R⁶ is fluoro.

In exemplified embodiments of Formula XII, R^{6″} is hydrogen, R^{6′} and R⁶are tert-butyl, and R⁶ is fluoro.

In certain embodiments, the disclosure relates to a compound of Formula XIII,

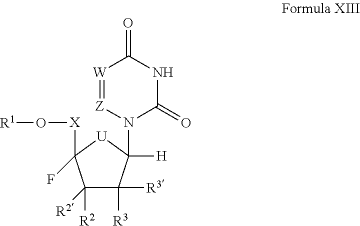

or a pharmaceutical or physiological salt thereof, wherein
X is CH₂, CHMe, CMe₂, CHF, CF₂, or CD₂;
U is S, NH, NR₇, CH₂, CHF, CF₂, CCH₂, or CCF₂;
W is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R″ is optionally substituted with one or more, the same or different, R¹⁰;
R¹ is selected from H,

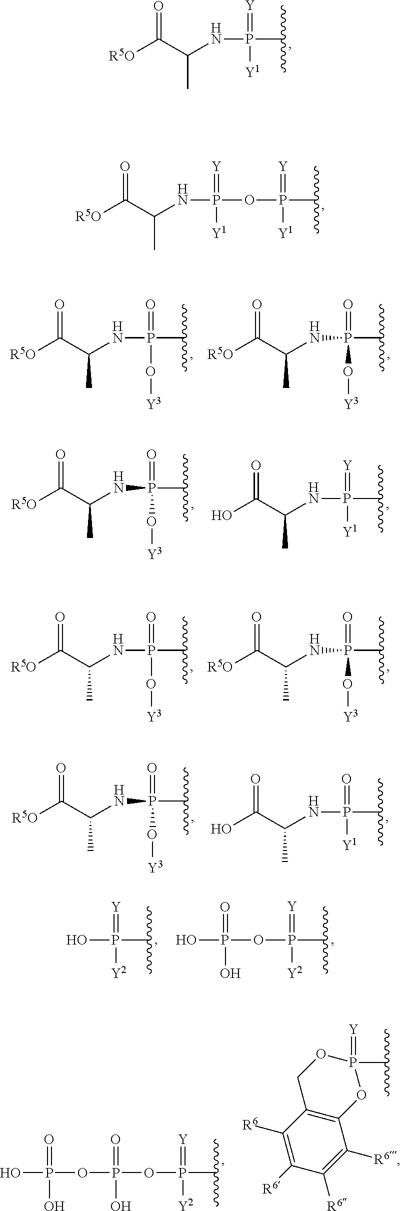

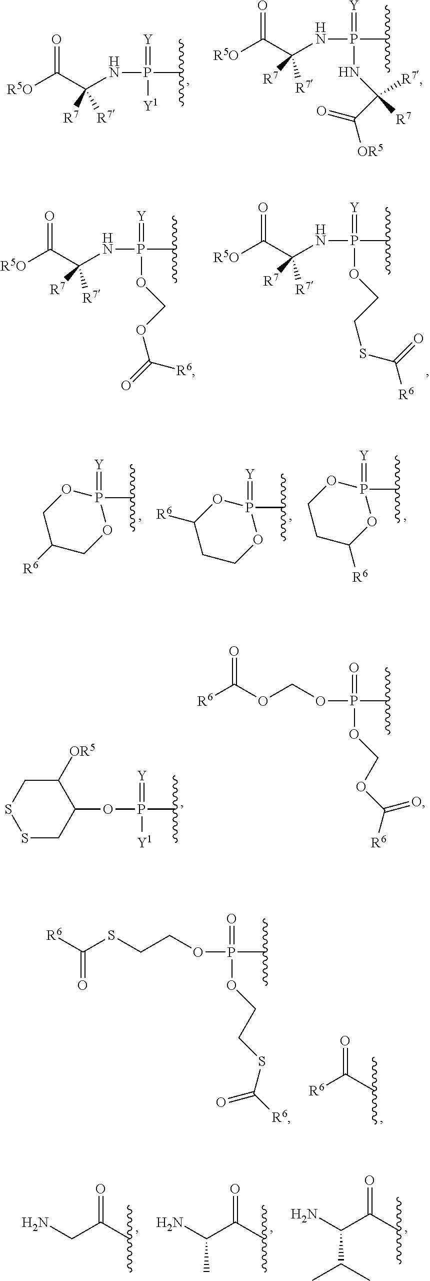

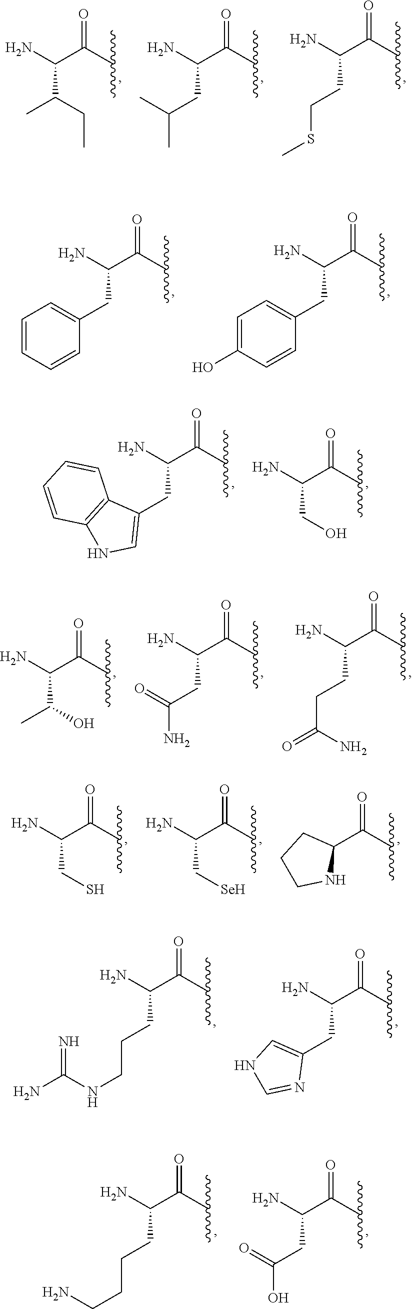

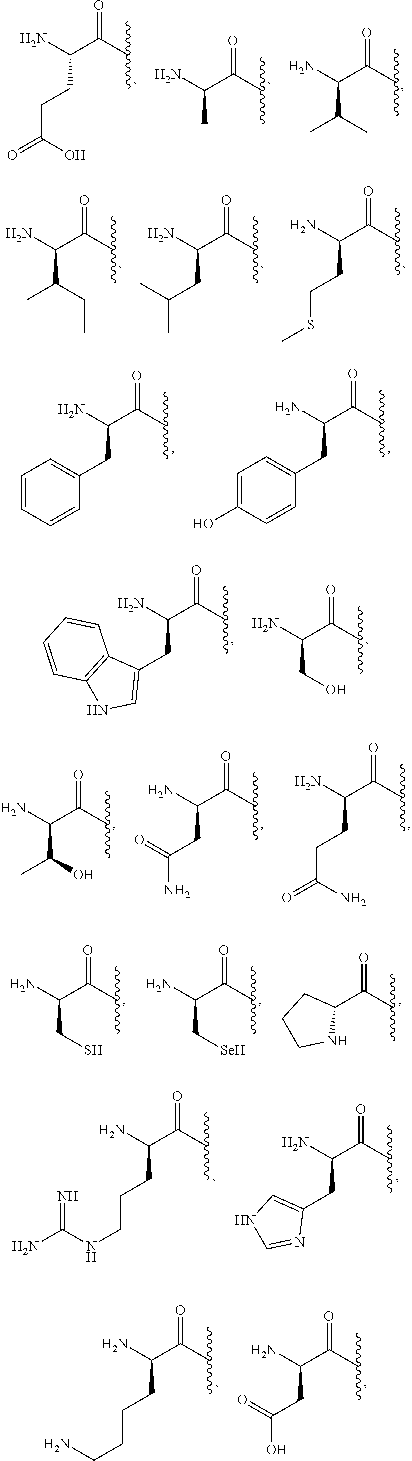

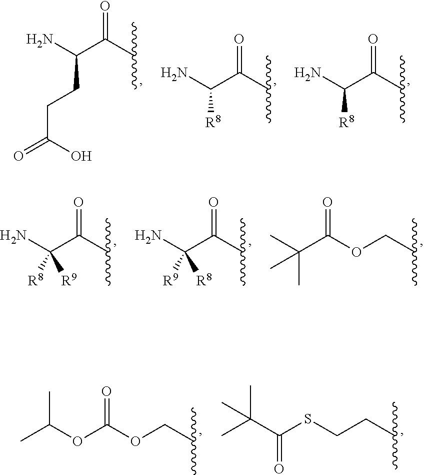

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R², R^{2′}, R³, R^{3′} are each independently selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R², R^{2′}, R³, R^{3′} are optionally substituted with one or more, the same or different, R¹⁰;
R² and R^{2′} can form a ring with the carbon they are attached to, wherein the ring is optionally substituted with one or more, the same or different, R¹⁰;
R³ and R^{3′} can form a ring with the carbon they are attached to, wherein the ring is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula XIII, R¹ is hydrogen, OH

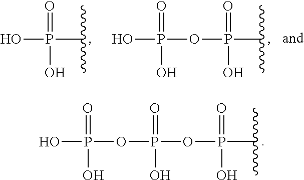

In exemplified embodiments of Formula XIII, X is CH₂.

In exemplified embodiments of Formula XIII, W is CR′.

In exemplified embodiments of Formula XIII, Z is CR″.

In exemplified embodiments of Formula XIII, R′ is H, F, Cl, OH, methyl, hydroxymethyl, fluoromethyl, difluoromethyl, trifluoromethyl, vinyl, ethynyl, formyl.

In exemplified embodiments of Formula XIII, R″ is H, F, Cl, OH, methyl, hydroxymethyl, fluoromethyl, difluoromethyl, trifluoromethyl, vinyl, ethynyl, formyl.

In exemplified embodiments of Formula XIII, R², R^{2′}, R³, R^{3′} are hydrogen, hydroxyl, amino, fluoro, chloro, cyano, methyl, fluoromethyl, methoxy, vinyl, ethynyl, and chloroethynyl.

In exemplified embodiments of Formula XIII, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XIII, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XIII, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XIII, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XIII, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula XIV,

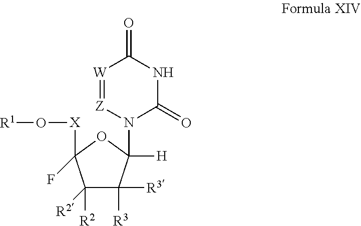

or a pharmaceutical or physiological salt thereof, wherein
X is CHMe, CMe₂, CHF, CF₂, or CD₂;
W is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R″ is optionally substituted with one or more, the same or different, R¹⁰;
R¹ is selected from H

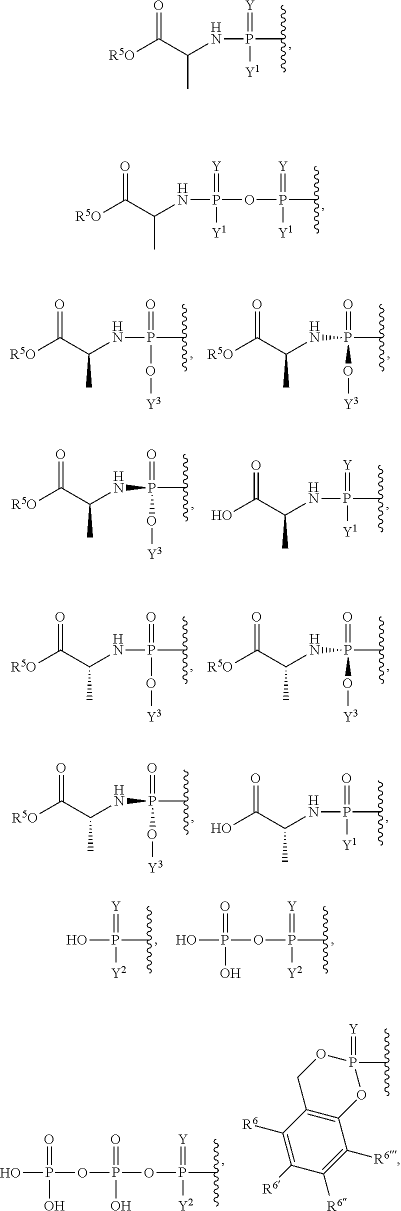

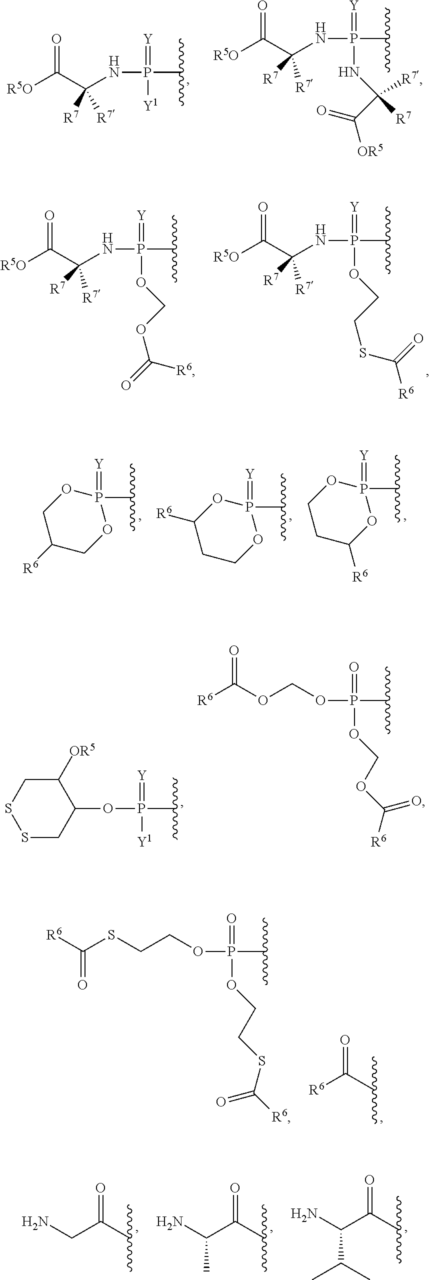

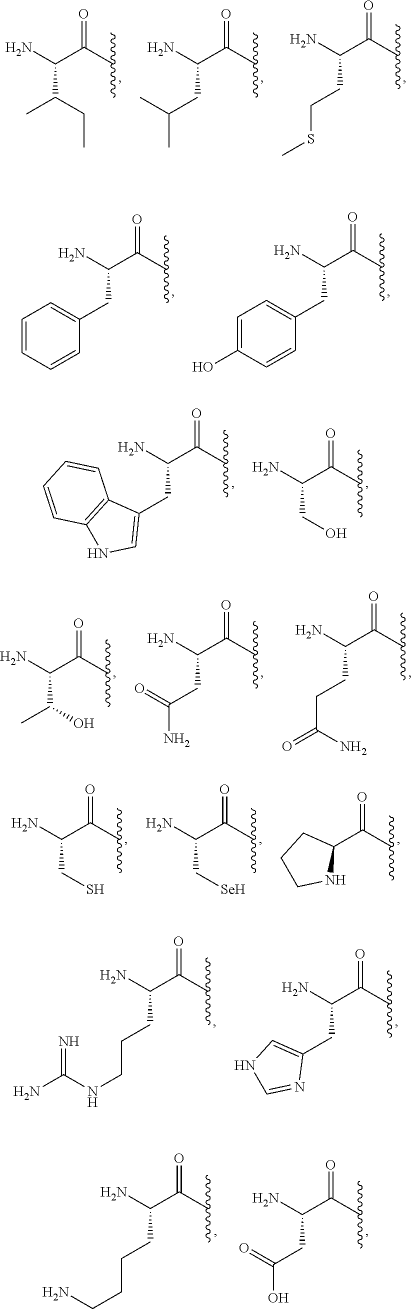

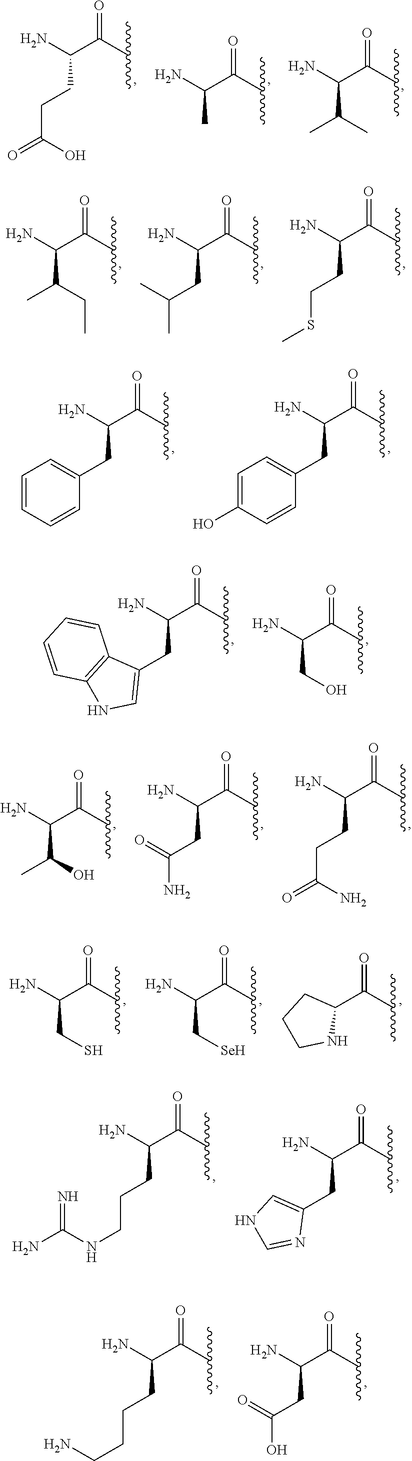

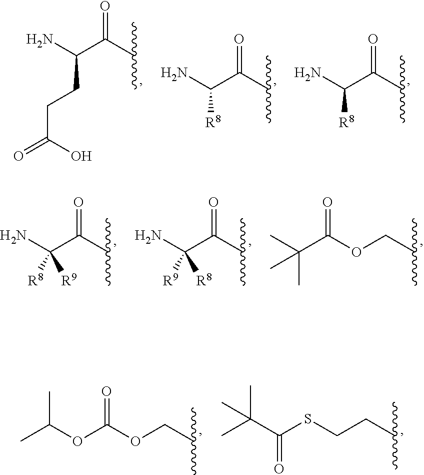

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R², R^{2′}, R³, R^{3′} are each independently selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R², R^{2′}, R³, R^{3′} are optionally substituted with one or more, the same or different, R¹⁰;
R² and R^{2′} can form a ring with the carbon they are attached to, wherein the ring is optionally substituted with one or more, the same or different, R¹⁰;
R³ and R^{3′} can form a ring with the carbon they are attached to, wherein the ring is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula XIV, R¹ is hydrogen,

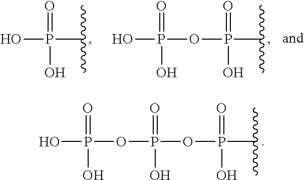

In exemplified embodiments of Formula XIV, W is CR′.

In exemplified embodiments of Formula XIV, Z is CR″.

In exemplified embodiments of Formula XIV, R′ is H, F, Cl, OH, methyl, hydroxymethyl, fluoromethyl, difluoromethyl, trifluoromethyl, vinyl, ethynyl, formyl.

In exemplified embodiments of Formula XIV, R″ is H, F, Cl, OH, methyl, hydroxymethyl, fluoromethyl, difluoromethyl, trifluoromethyl, vinyl, ethynyl, formyl.

In exemplified embodiments of Formula XIV, R², R^{2′}, R³, R^{3′} are hydrogen, hydroxyl, amino, fluoro, chloro, cyano, methyl, fluoromethyl, methoxy, vinyl, ethynyl, and chloroethynyl.

In exemplified embodiments of Formula XIV, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XIV, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XIV, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XIV, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XIV, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula XV,

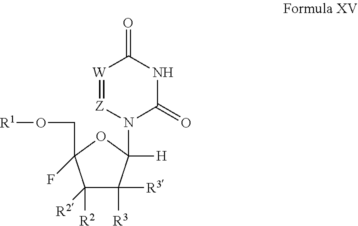

or a pharmaceutical or physiological salt thereof, wherein
W is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R″ is optionally substituted with one or more, the same or different, R¹⁰;
R¹ is selected from H,

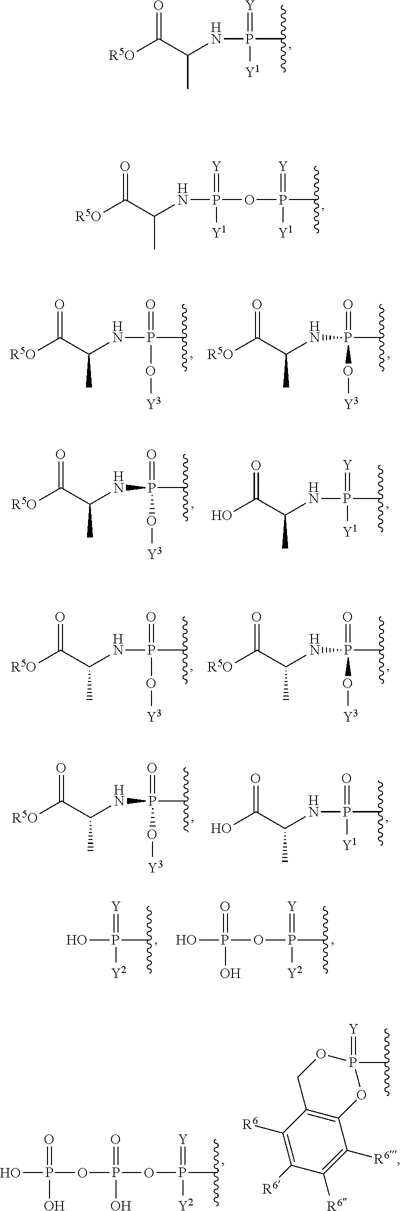

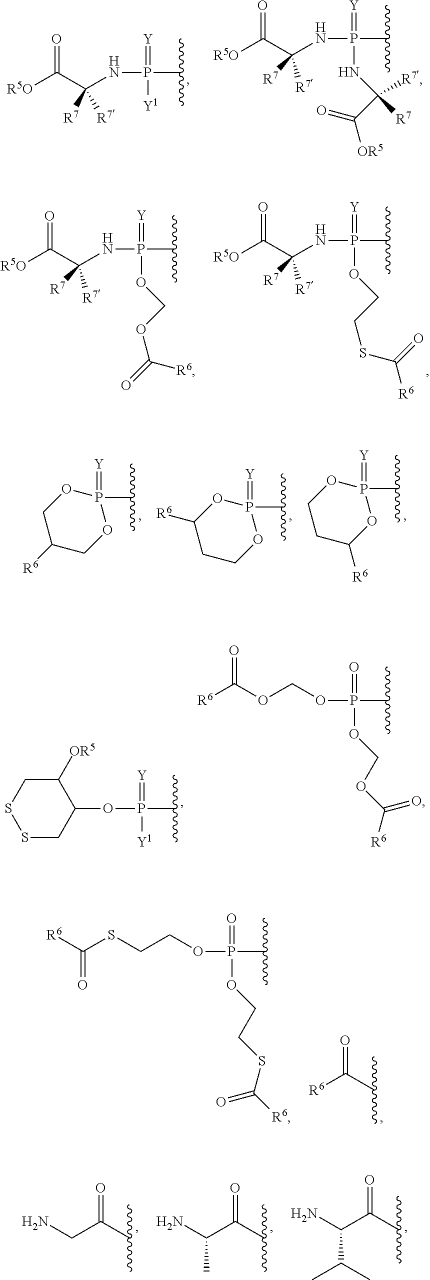

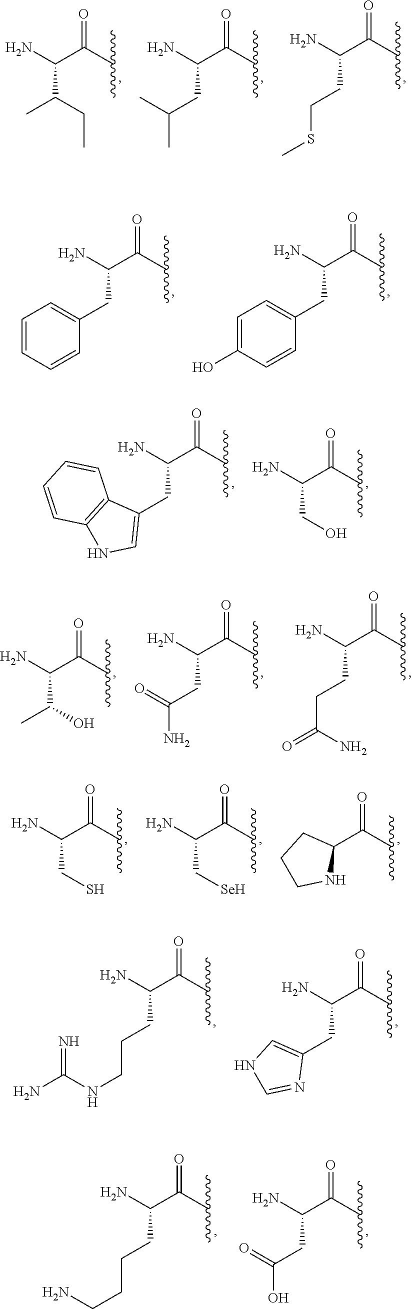

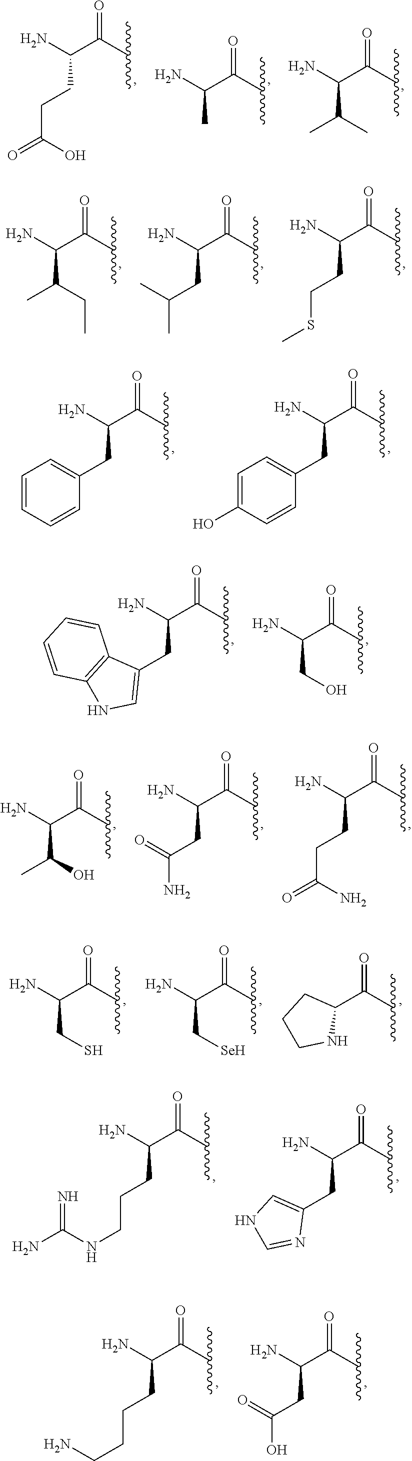

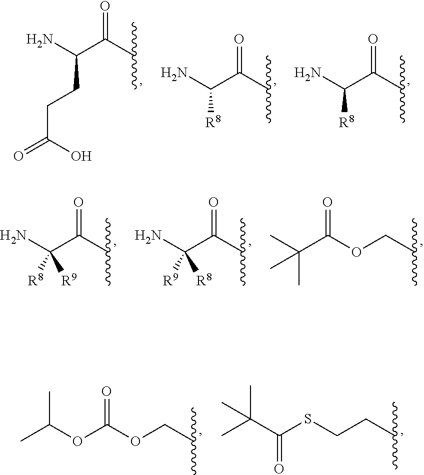

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R² is selected from deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R² is optionally substituted with one or more, the same or different, R¹⁰;
R^{2′} is selected from deuterium, alkyl, alkenyl, alkynyl, allenyl, alkoxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R^{2′} is optionally substituted with one or more, the same or different, R¹⁰;
R³ is selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R³ is optionally substituted with one or more, the same or different, R¹⁰;
R^{3′} is selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R^{3′} is optionally substituted with one or more, the same or different, R¹⁰;
R² and R^{2′} can form a ring with the carbon they are attached to, wherein the ring is optionally substituted with one or more, the same or different, R¹⁰;
R³ and R^{3′} can form a ring with the carbon they are attached to, wherein the ring is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula XV, R¹ is hydrogen,

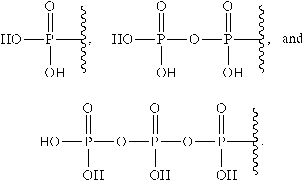

In exemplified embodiments of Formula XV, W is CR′.

In exemplified embodiments of Formula XV, Z is CR″.

In exemplified embodiments of Formula XV, R′ is H, F, Cl, OH, methyl, hydroxymethyl, fluoromethyl, difluoromethyl, trifluoromethyl, vinyl, ethynyl, formyl.

In exemplified embodiments of Formula XV, R″ is H, F, Cl, OH, methyl, hydroxymethyl, fluoromethyl, difluoromethyl, trifluoromethyl, vinyl, ethynyl, formyl.

In exemplified embodiments of Formula XV, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XV, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XV, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XV, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XV, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula XVI,

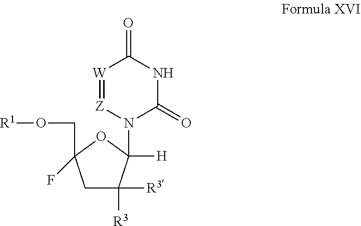

or a pharmaceutical or physiological salt thereof, wherein
W is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R″ is optionally substituted with one or more, the same or different, R¹⁰;
R¹ is selected from H,

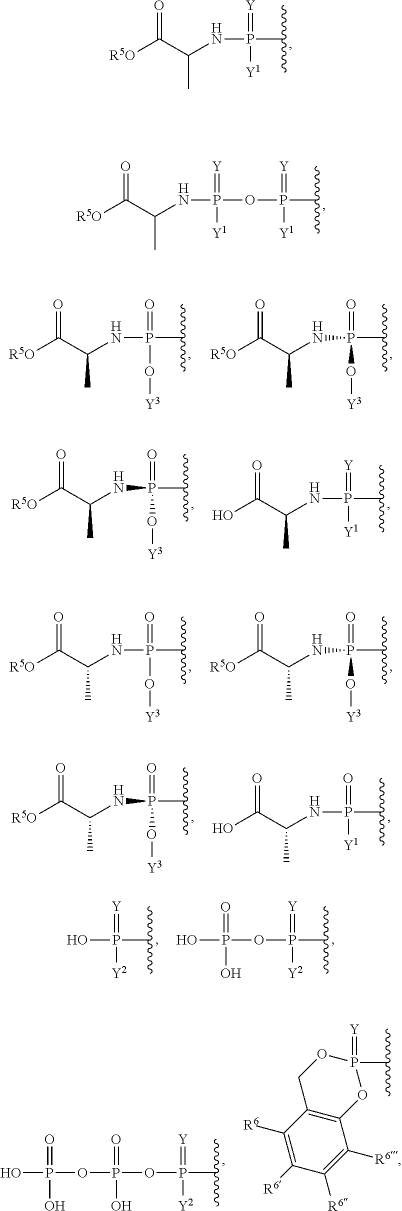

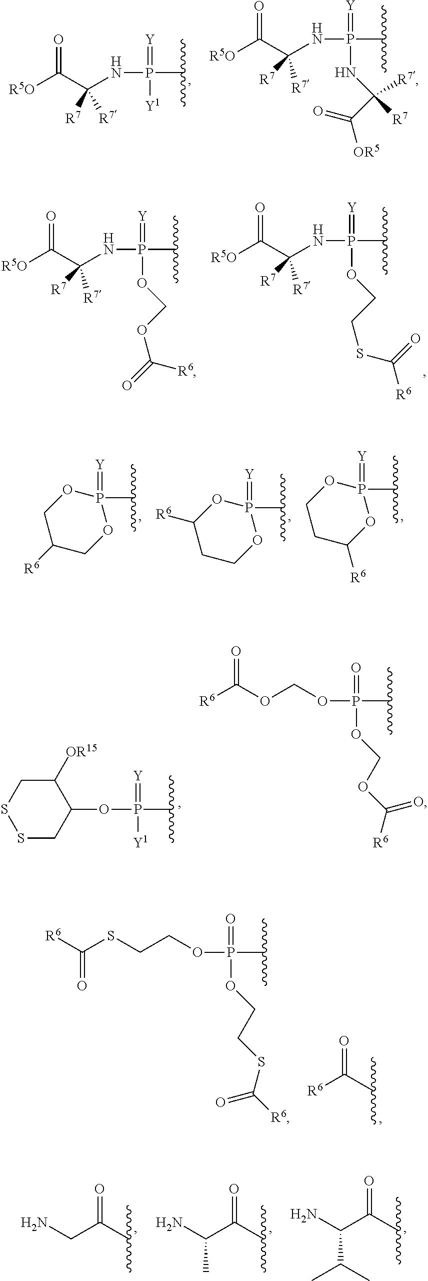

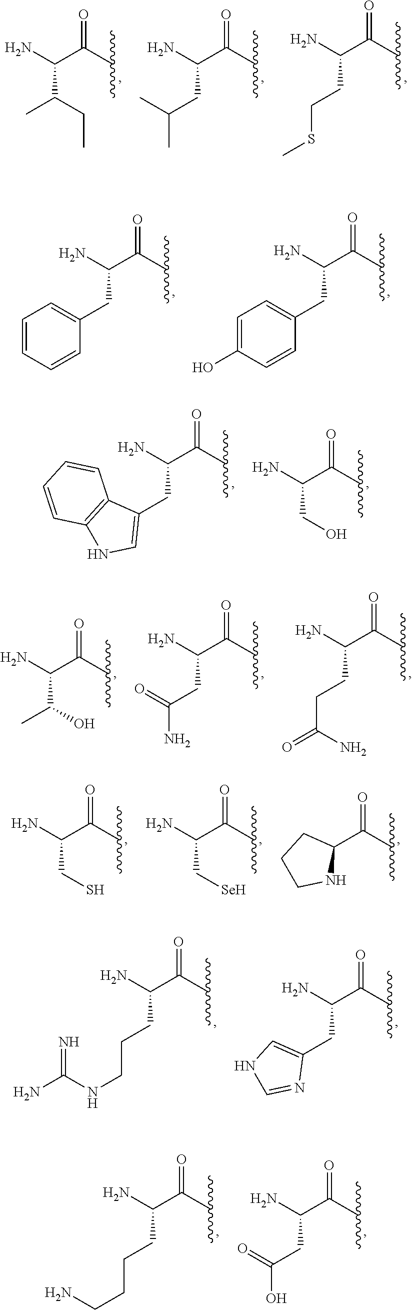

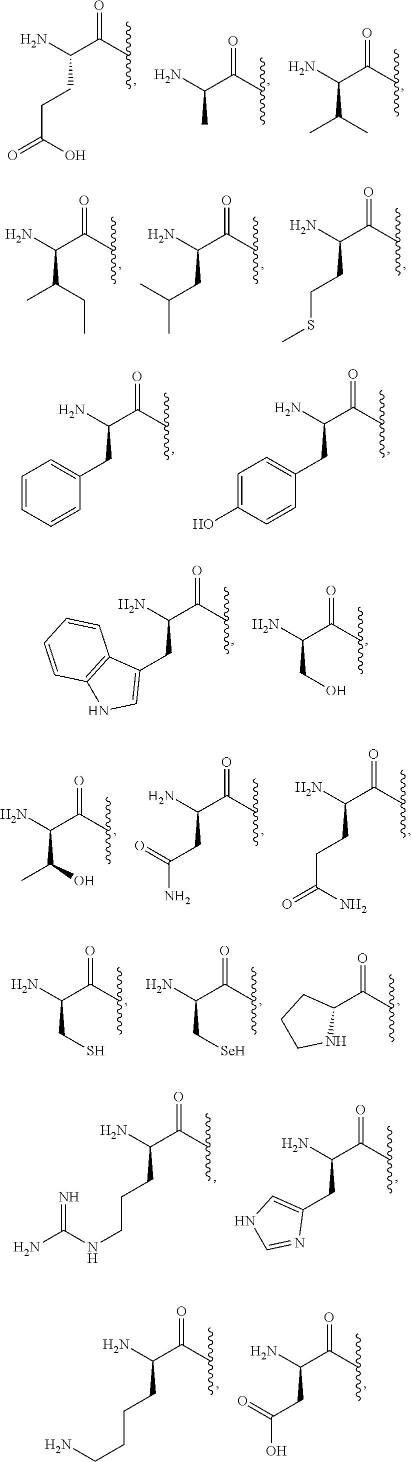

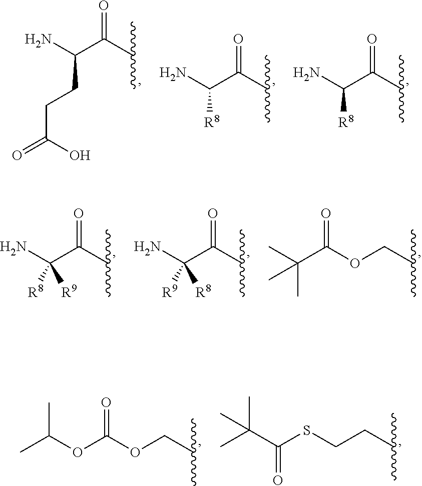

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R³ is selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R³ is optionally substituted with one or more, the same or different, R¹⁰;
R^{3′} is selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R^{3′} is optionally substituted with one or more, the same or different, R¹⁰;
R³ and R^{3′} can form a ring with the carbon they are attached to, wherein the ring is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

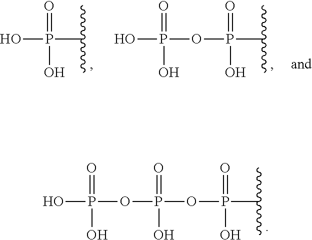 [SMILES: *P(=O)(O)O ; *P(=O)(O)OP(=O)(O)O ; *P(=O)(O)OP(=O)(O)OP(=O)(O)O]

In exemplified embodiments of Formula XVI, W is CR′.

In exemplified embodiments of Formula XVI, Z is CR″.

In exemplified embodiments of Formula XVI, R′ is H, F, Cl, OH, methyl, hydroxymethyl, fluoromethyl, difluoromethyl, trifluoromethyl, vinyl, ethynyl, formyl.

In exemplified embodiments of Formula XVI, R″ is H, F, Cl, OH, methyl, hydroxymethyl, fluoromethyl, difluoromethyl, trifluoromethyl, vinyl, ethynyl, formyl.

In exemplified embodiments of Formula XVI, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XVI, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XVI, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XVI, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XVI, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula XVII,

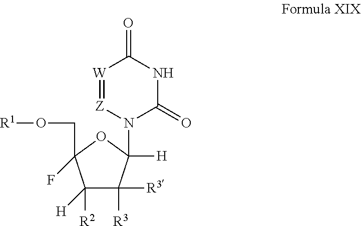

or a pharmaceutical or physiological salt thereof, wherein
W is N or CR′;
Z is N or CR″;
R′ is deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R′ is optionally substituted with one or more, the same or different, R¹⁰;
R″ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R″ is optionally substituted with one or more, the same or different, R¹⁰;
R¹ is selected from H,

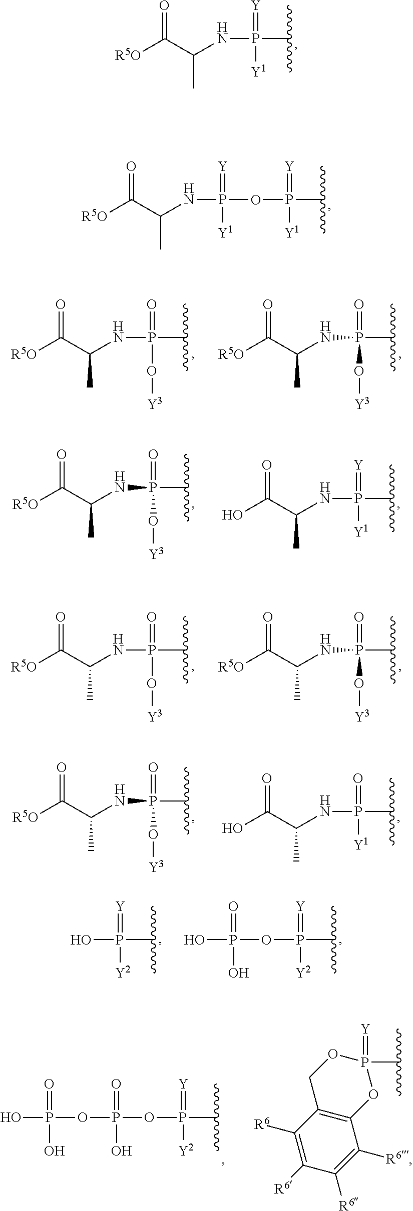

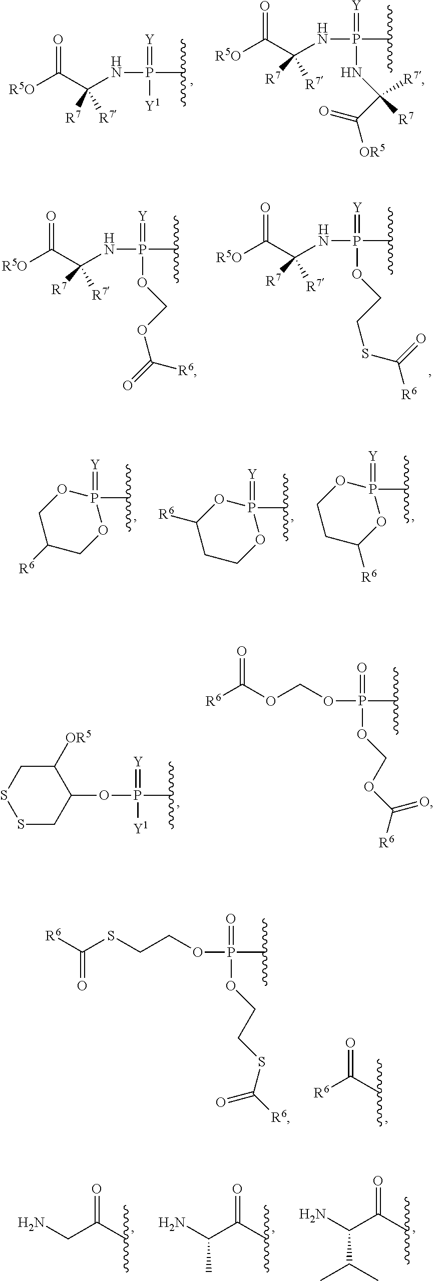

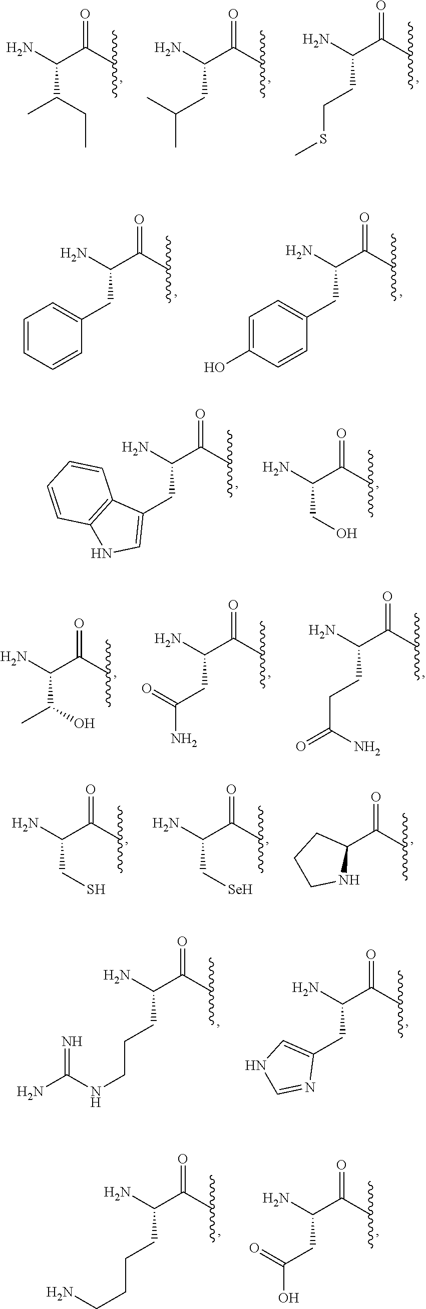

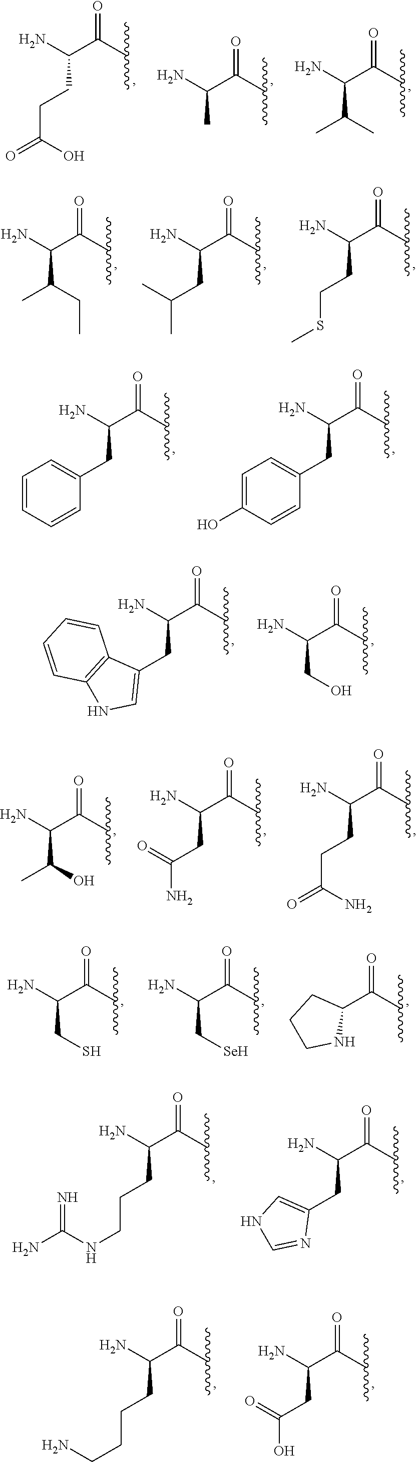

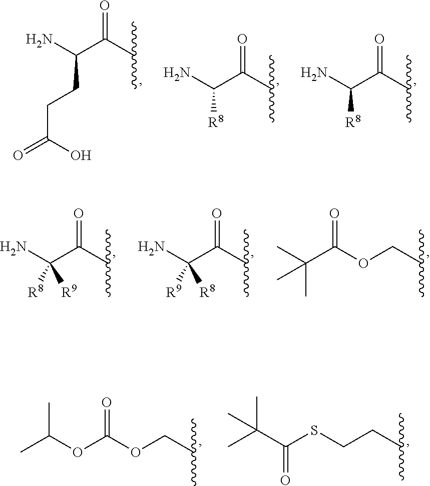

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R² is selected from deuterium, alkyl, alkenyl, alkynyl, allenyl, alkoxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R² is optionally substituted with one or more, the same or different, R¹⁰;
R³ and R^{3′} are each independently selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R³ and R^{3′} are optionally substituted with one or more, the same or different, R¹⁰;
R³ and R^{3′} can form a ring with the carbon they are attached to, wherein the ring is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula XVII, R¹ is hydrogen,

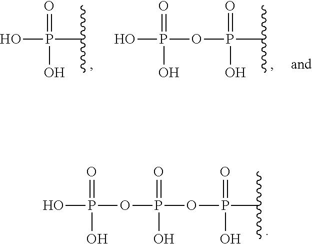 [SMILES: *P(=O)(O)O ; *P(=O)(O)OP(=O)(O)O ; *P(=O)(O)OP(=O)(O)OP(=O)(O)O]

In exemplified embodiments of Formula XVII, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XVII, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XVII, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XVII, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XVII, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula XVIII,

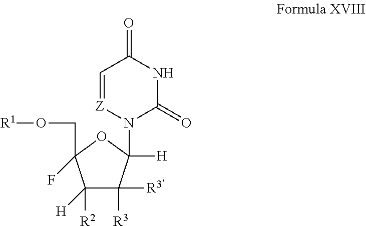

or a pharmaceutical or physiological salt thereof, wherein
Z is N or CR″;
R″ is deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R″ is optionally substituted with one or more, the same or different, R¹⁰;
R¹ is selected from H

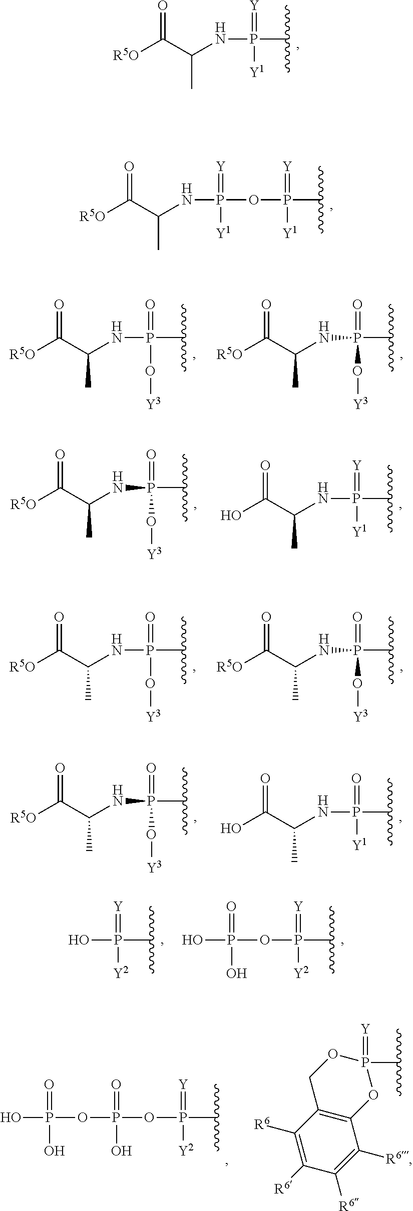

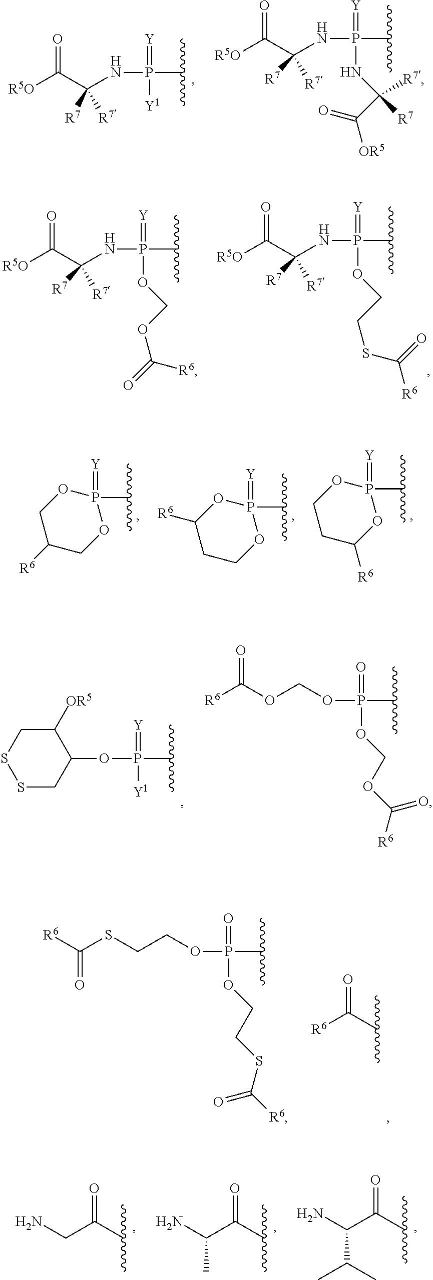

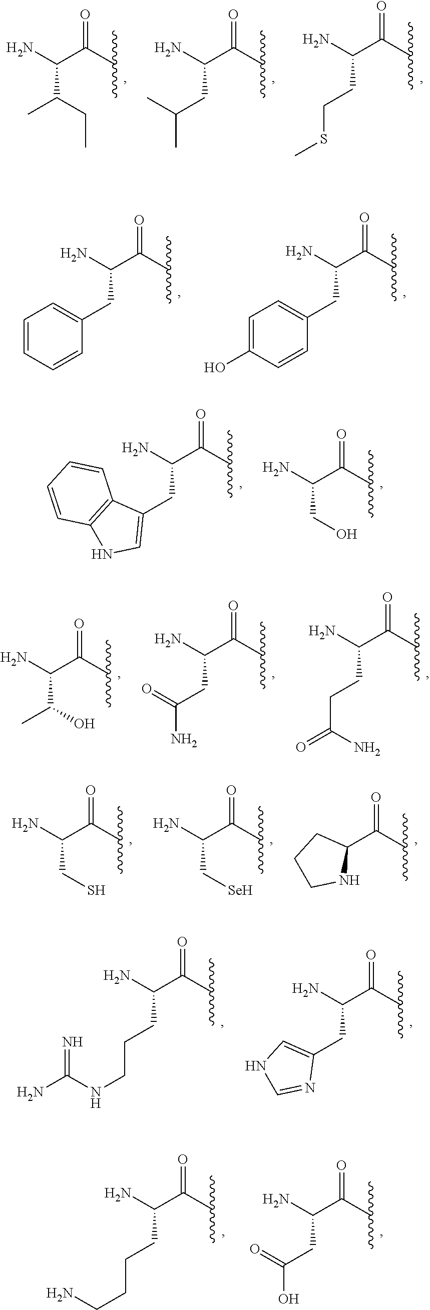

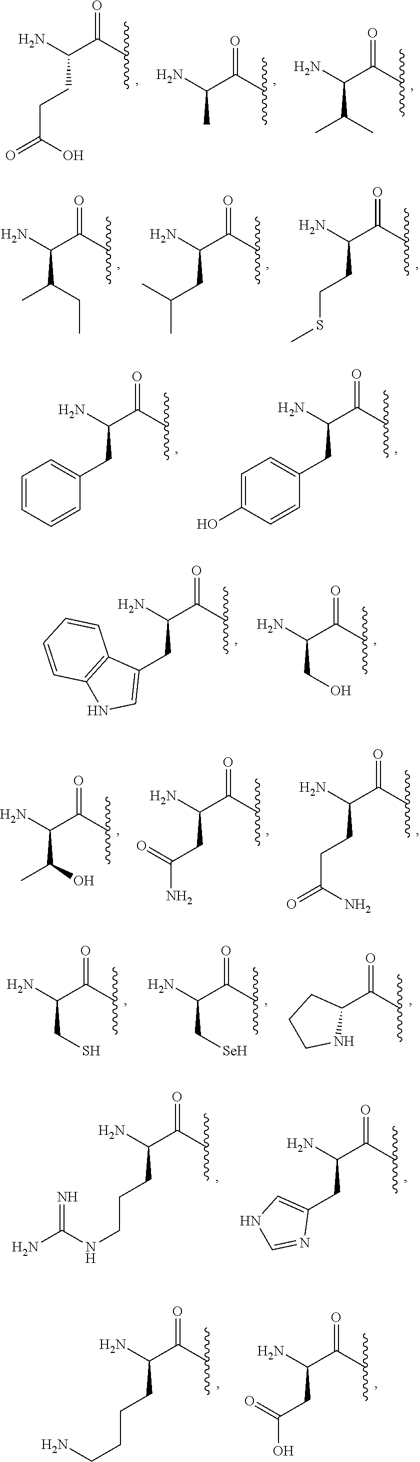

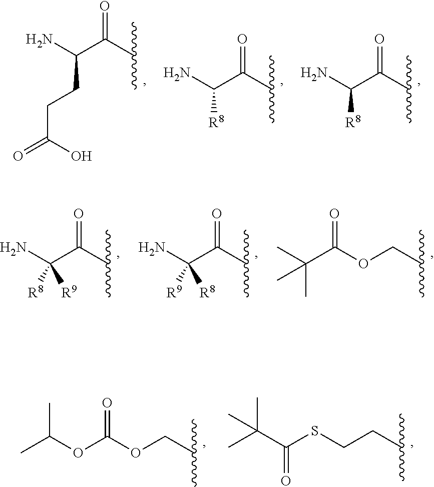

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R² is selected from deuterium, alkyl, alkenyl, alkynyl, allenyl, alkoxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R² is optionally substituted with one or more, the same or different, R¹⁰;
R³ and R^{3′} are each independently selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R³ and R^{3′} are optionally substituted with one or more, the same or different, R¹⁰;
R³ and R^{3′} can form a ring with the carbon they are attached to, wherein the ring is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula XVIII, R¹ is hydrogen,

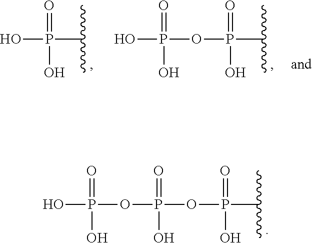 [SMILES: *P(=O)(O)O ; *P(=O)(O)OP(=O)(O)O ; *P(=O)(O)OP(=O)(O)OP(=O)(O)O]

In exemplified embodiments of Formula XVIII, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XVIII, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XVIII, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XVIII, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XVIII, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula XIX,

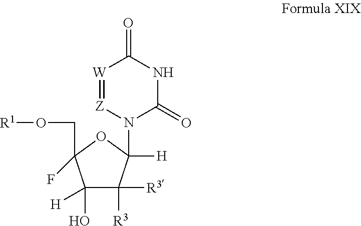

or a pharmaceutical or physiological salt thereof, wherein
W is N or CR′;
Z is N or CR″;
R′ is hydrogen, deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R′ is optionally substituted with one or more, the same or different, R¹⁰;
R″ is deuterium, halogen, hydroxyl, amino, thiol, alkyl, alkenyl, alkynyl, aryl, heteroaryl, carbocyclyl, heterocarbocyclyl, cycloalkyl, heterocyclyl, or acyl, wherein R″ is optionally substituted with one or more, the same or different, R¹⁰;
R¹ is selected from H,

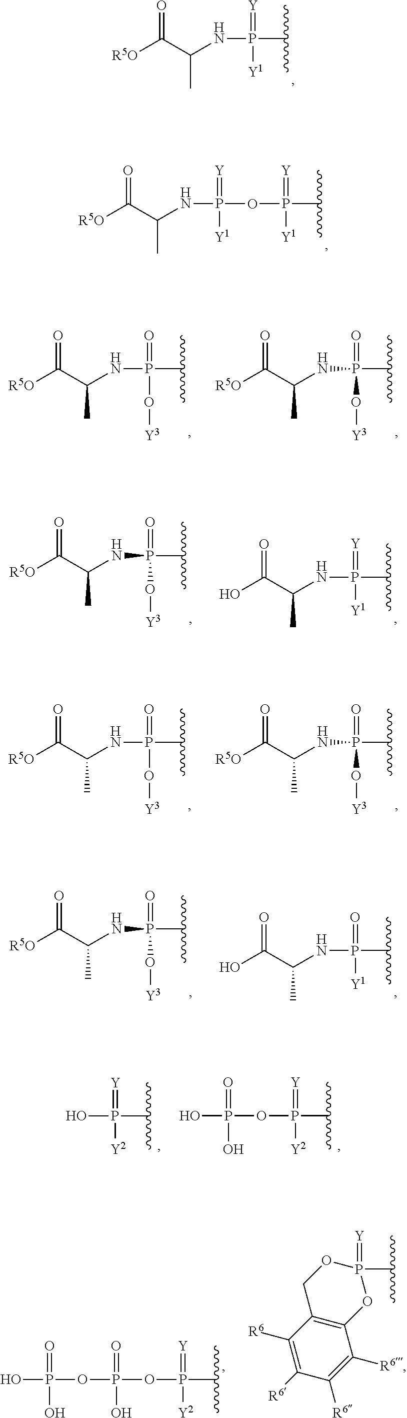

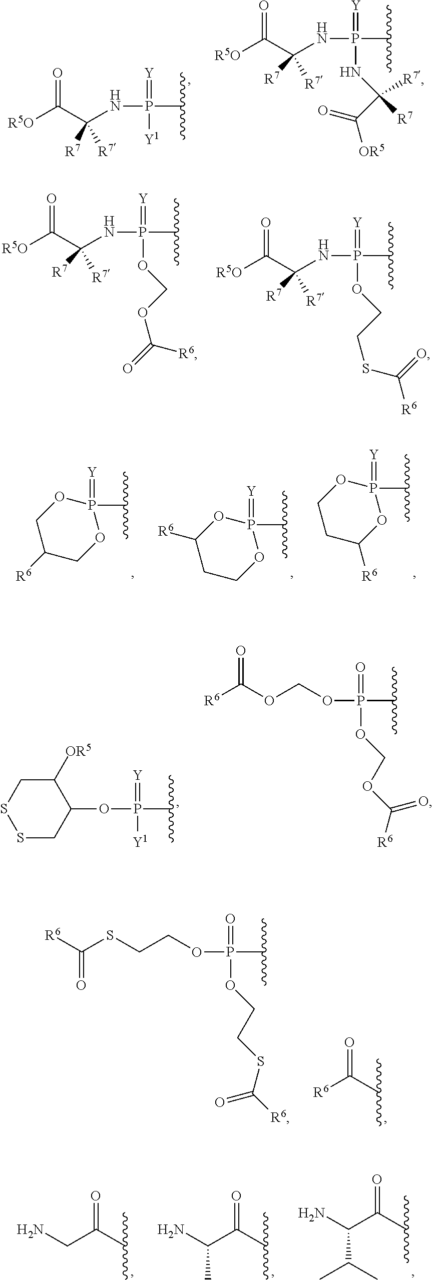

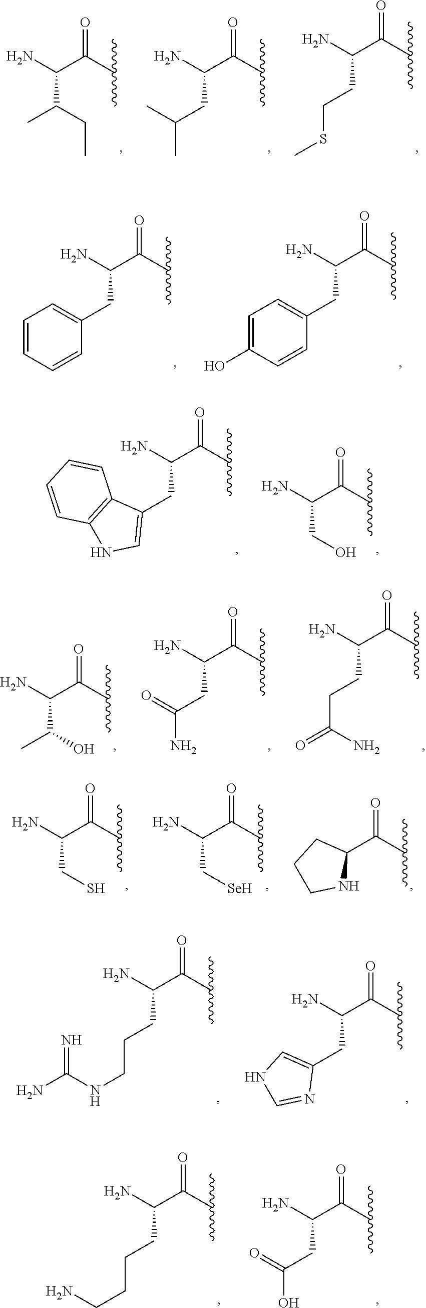

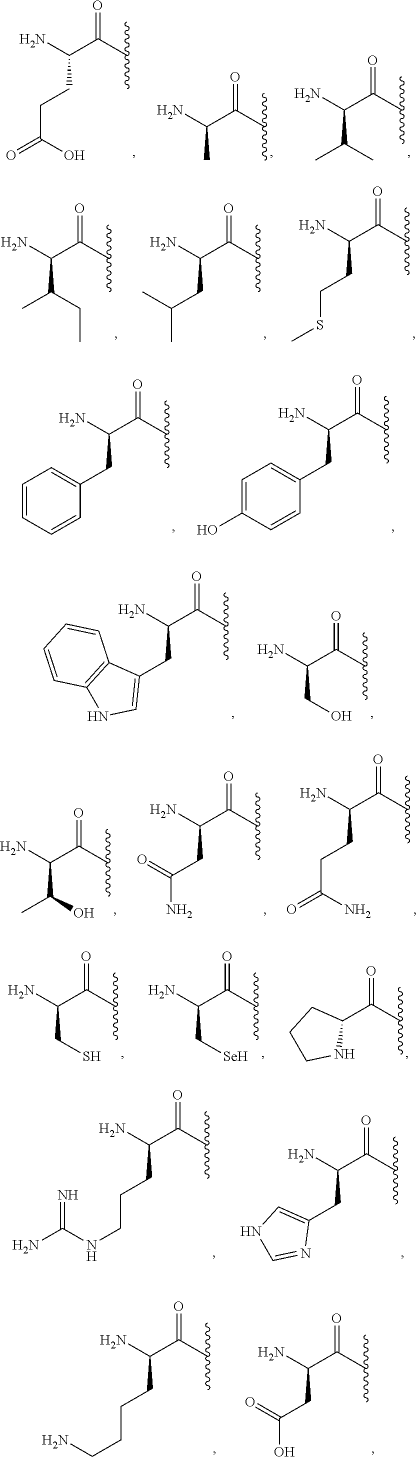

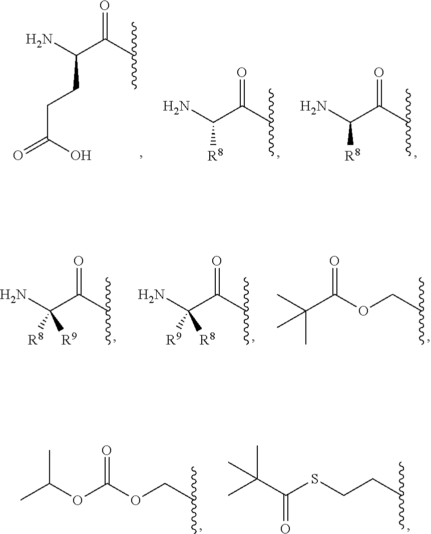

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R³ is selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R³ is optionally substituted with one or more, the same or different, R¹⁰;
R^{3′} is selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R^{3′} is optionally substituted with one or more, the same or different, R¹⁰;
R³ and R^{3′} can form a ring with the carbon they are attached to, wherein the ring is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula XIX, R¹ is hydrogen,

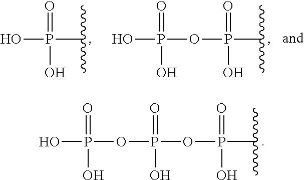

In exemplified embodiments of Formula XIX, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XIX, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XIX, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XIX, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XIX, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula XX,

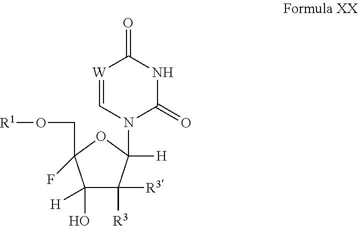

or a pharmaceutical or physiological salt thereof, wherein
W is N or CR′;
R′ is deuterium, chloro, iodo, hydroxyl, amino, thiol, alkyl, alkenyl, alkynyl, aryl, heteroaryl, carbocyclyl, heterocarbocyclyl, cycloalkyl, heterocyclyl, or acyl, wherein R′ is optionally substituted with one or more, the same or different, R¹⁰;
R¹ is selected from H,

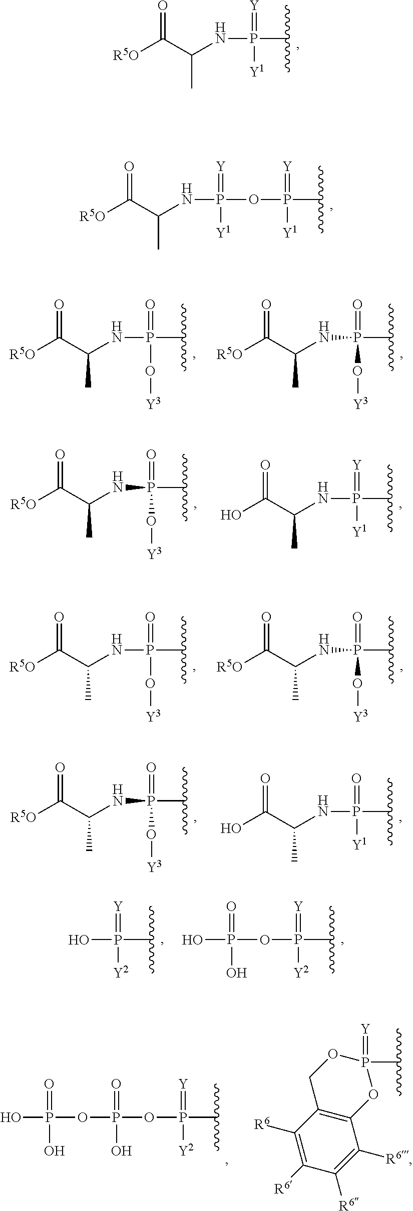

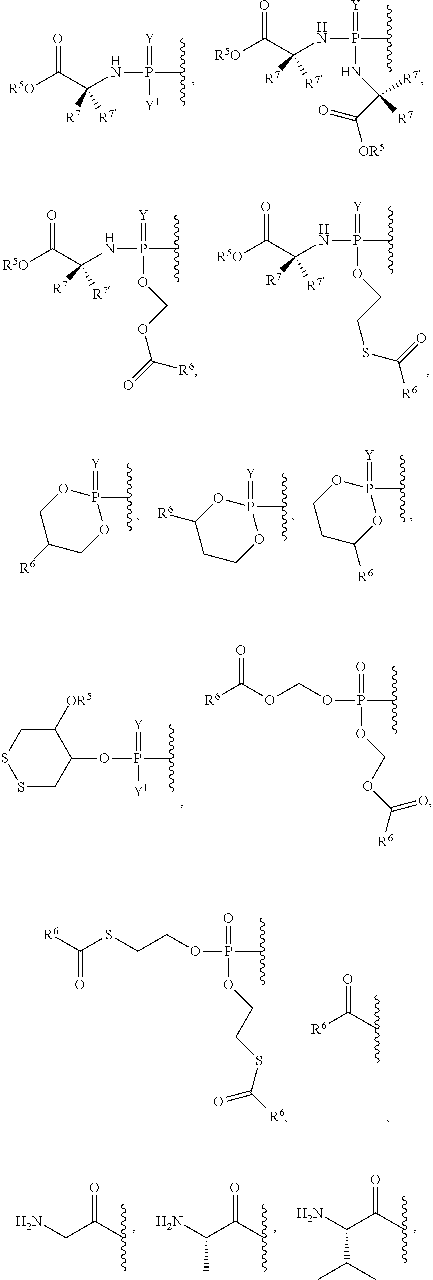

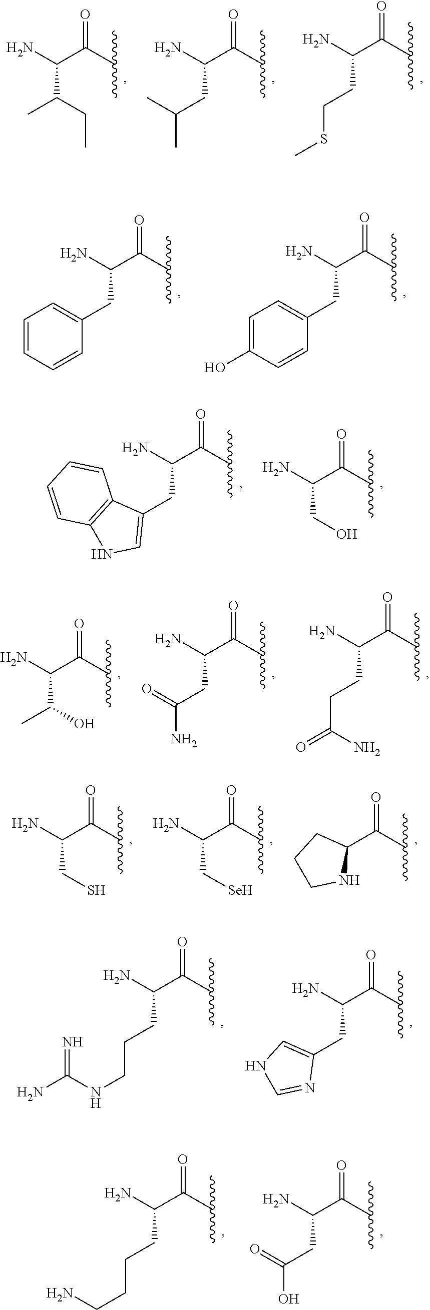

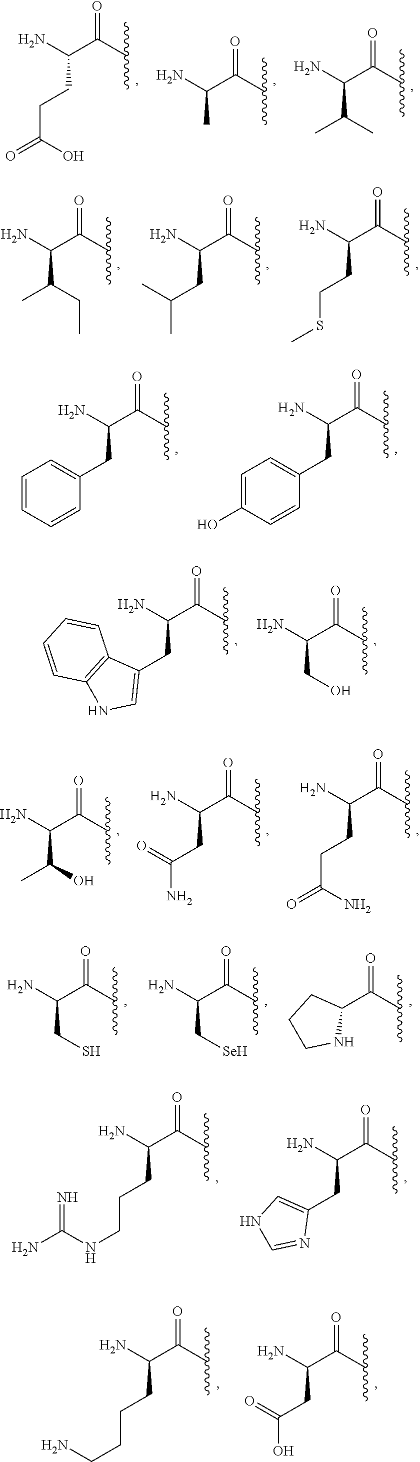

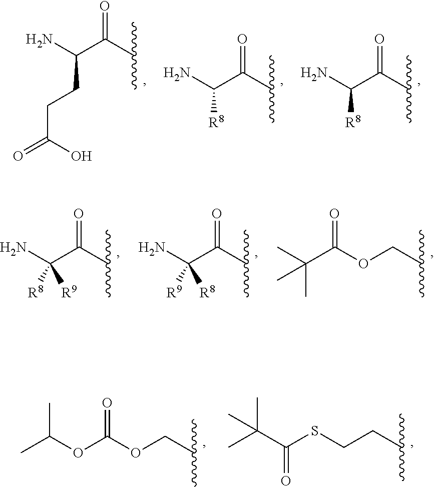

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R³ is selected from deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R³ is optionally substituted with one or more, the same or different, R¹⁰;
R^{3′} is selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R^{3′} is optionally substituted with one or more, the same or different, R¹⁰;
R³ and R^{3′} can form a ring with the carbon they are attached to, wherein the ring is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula XX, R¹ is hydrogen

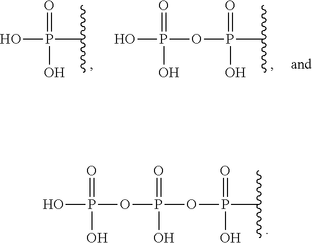 [SMILES: *P(=O)(O)O ; *P(=O)(O)OP(=O)(O)O ; *P(=O)(O)OP(=O)(O)OP(=O)(O)O]

In exemplified embodiments of Formula XX, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XX, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XX, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XX, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XX, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula XXI,

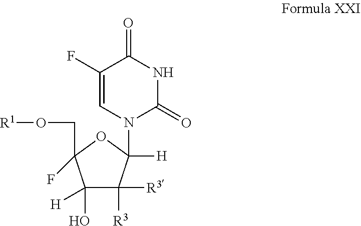

or a pharmaceutical or physiological salt thereof, wherein
R¹ is selected from H,

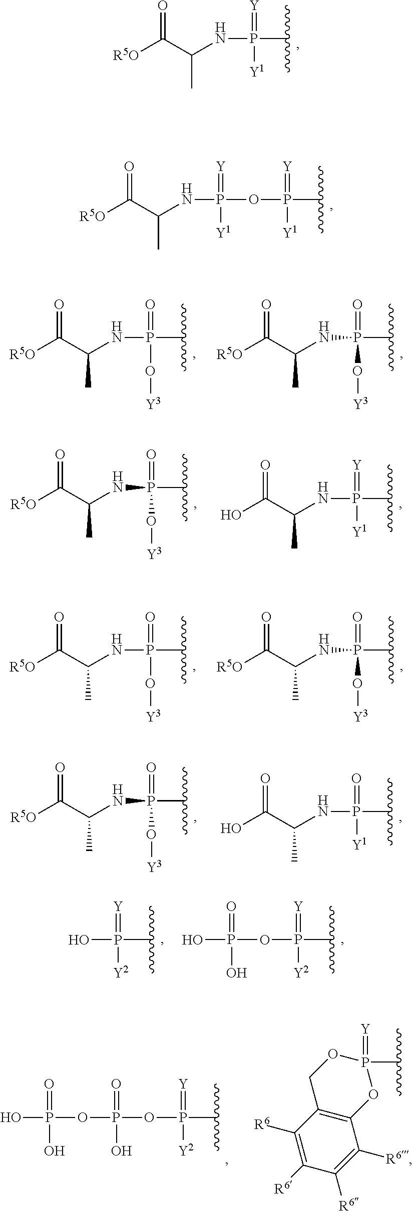

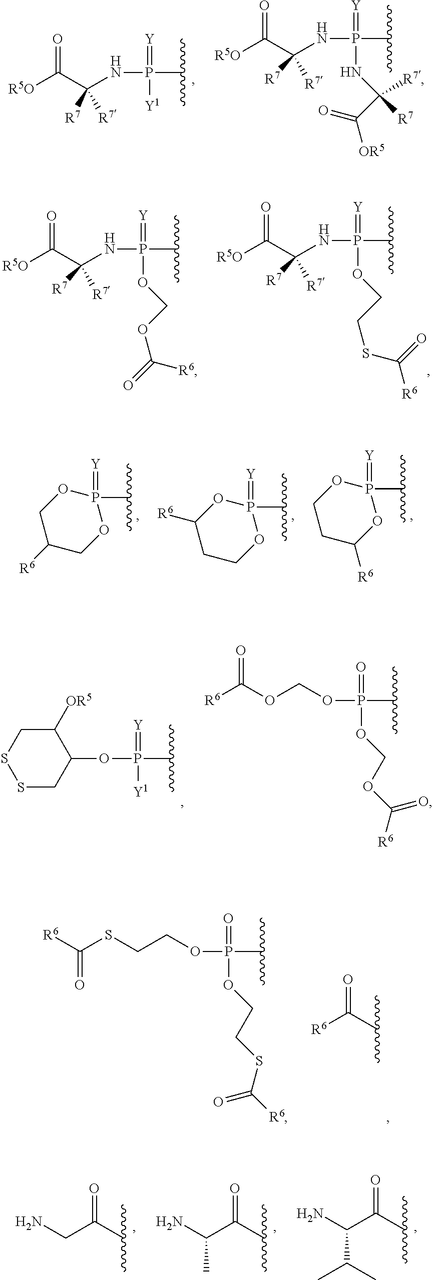

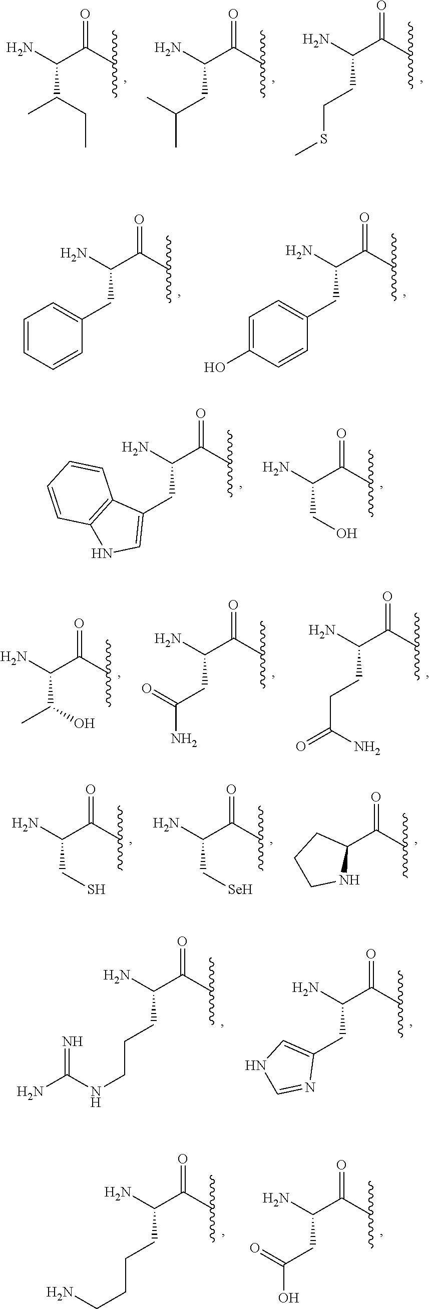

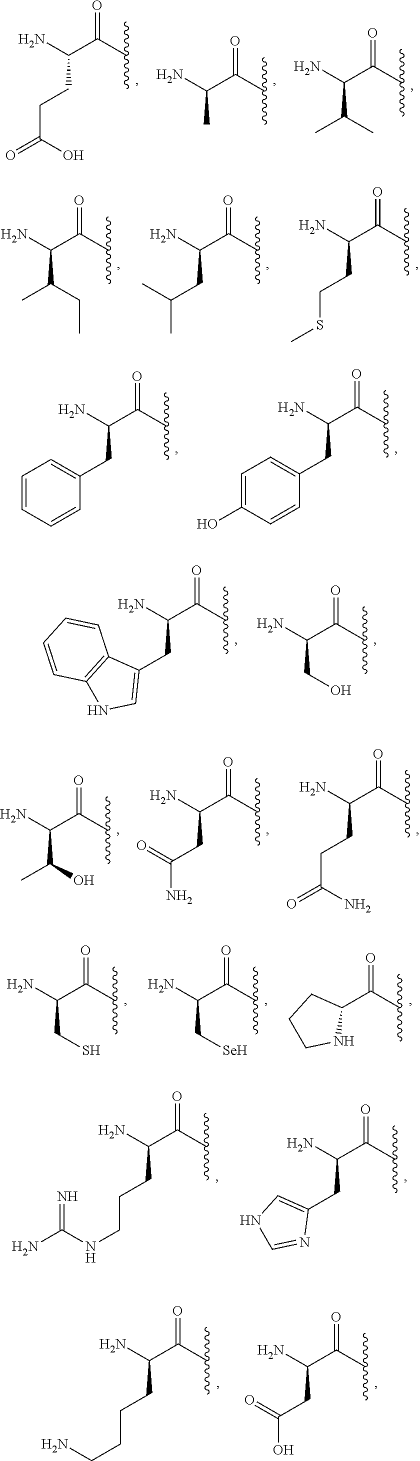

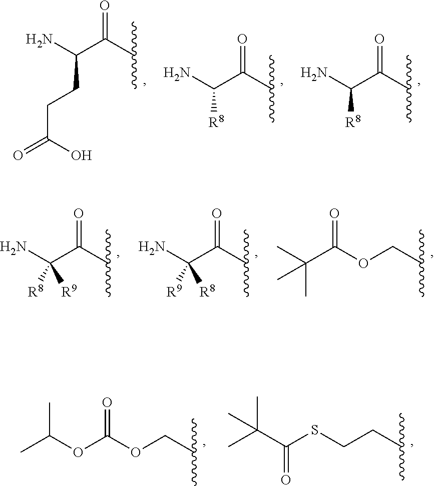

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R³ is selected from deuterium, alkyl, alkenyl, alkynyl, allenyl, alkoxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R³ is optionally substituted with one or more, the same or different, R¹⁰;
R^{3′} is selected from hydrogen, deuterium, alkyl, alkenyl, alkynyl, allenyl, alkoxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R^{3′} is optionally substituted with one or more, the same or different, R¹⁰;
R³ and R^{3′} can form a ring with the carbon they are attached to, wherein the ring is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula XXI, R¹ is hydrogen, OH,

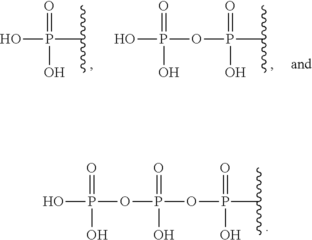 [SMILES: *P(=O)(O)O ; *P(=O)(O)OP(=O)(O)O ; *P(=O)(O)OP(=O)(O)OP(=O)(O)O]

In exemplified embodiments of Formula XXI, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXI, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXI, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXI, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXI, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula XXII,

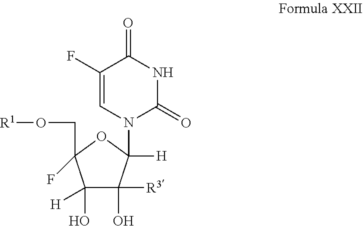

or a pharmaceutical or physiological salt thereof, wherein
R¹ is selected from H,

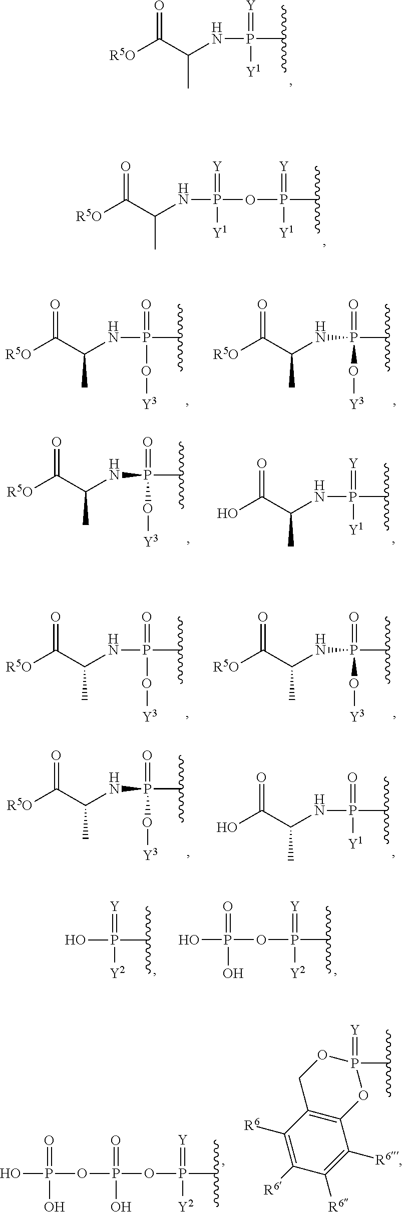

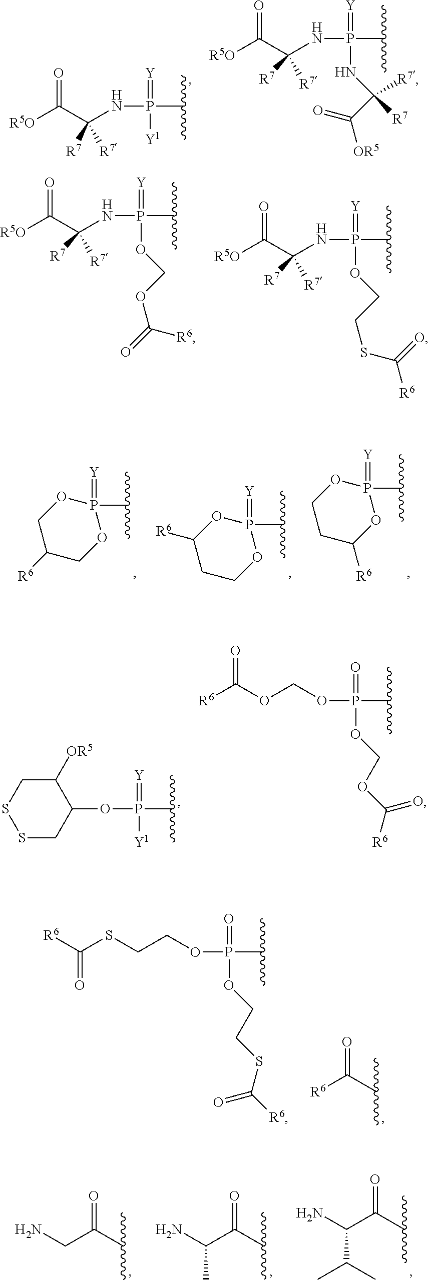

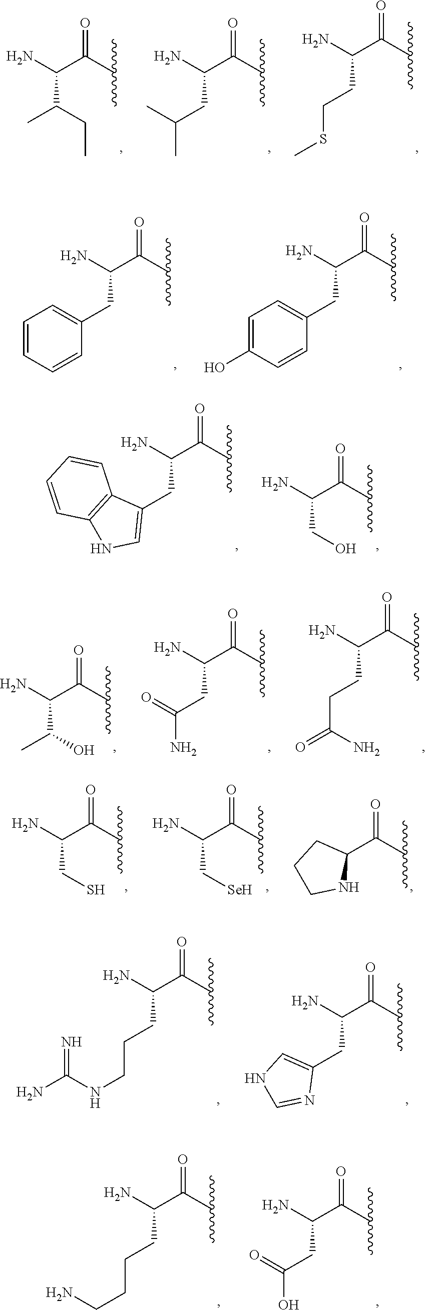

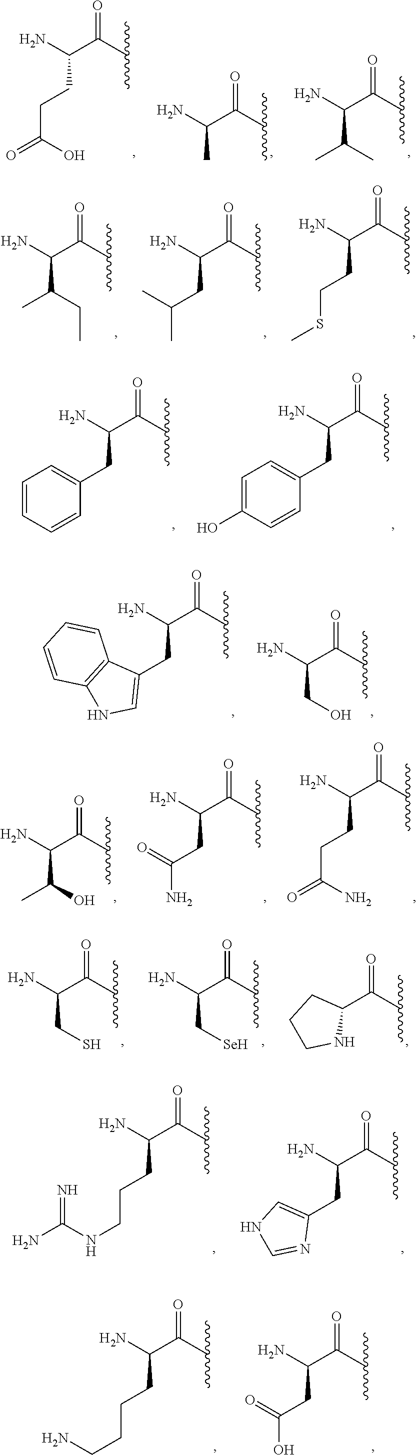

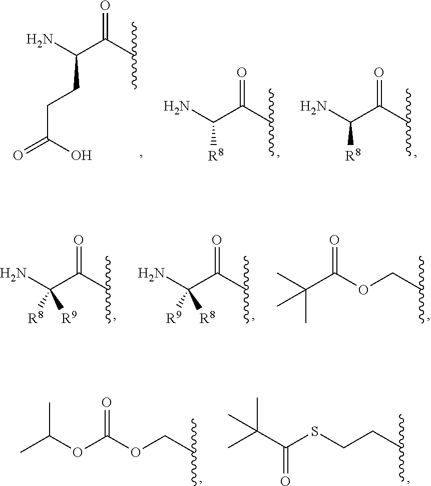

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R^{3′} is selected from deuterium, alkyl, alkenyl, alkynyl, allenyl, alkoxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R^{3′} is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula XXII, R¹ is hydrogen, OH

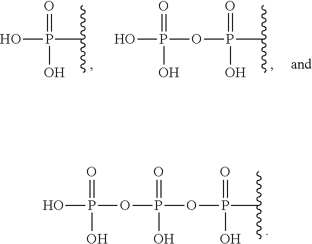 [SMILES: *P(=O)(O)O ; *P(=O)(O)OP(=O)(O)O ; *P(=O)(O)OP(=O)(O)OP(=O)(O)O]

In exemplified embodiments of Formula XXII, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXII, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXII, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXII, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXII, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula XXIII,

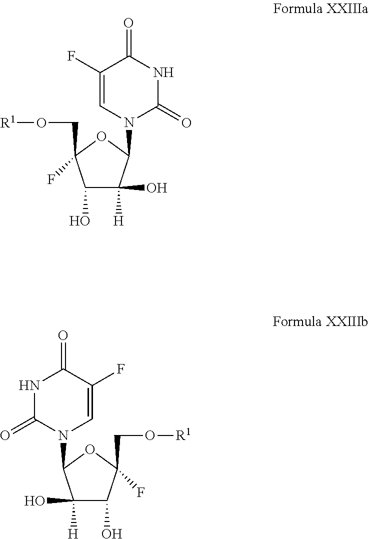

or a pharmaceutical or physiological salt thereof, wherein
R¹ is selected from H,

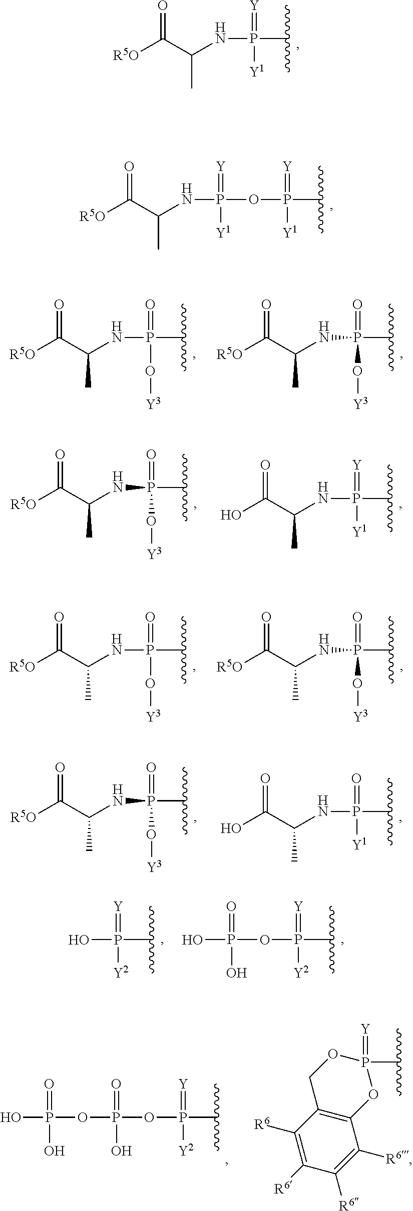

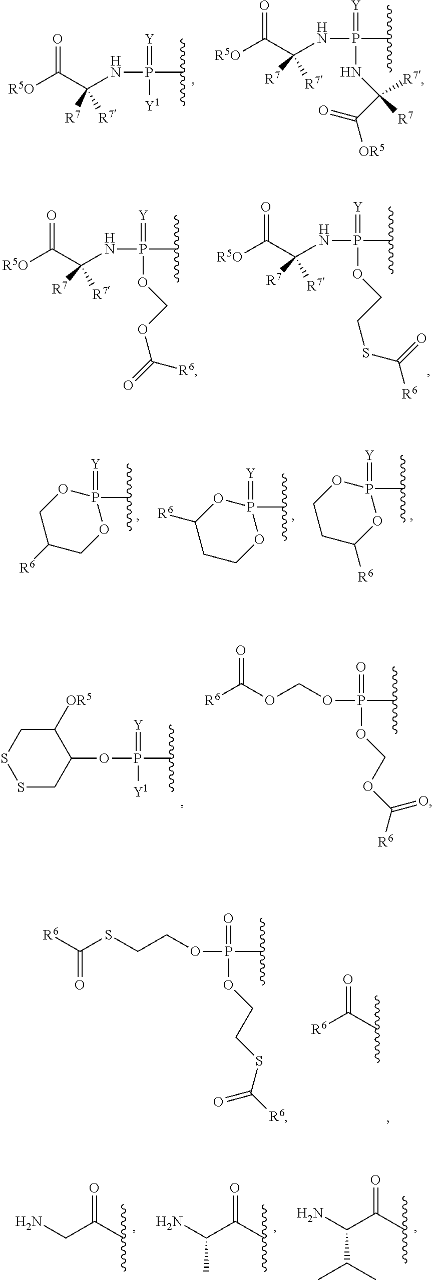

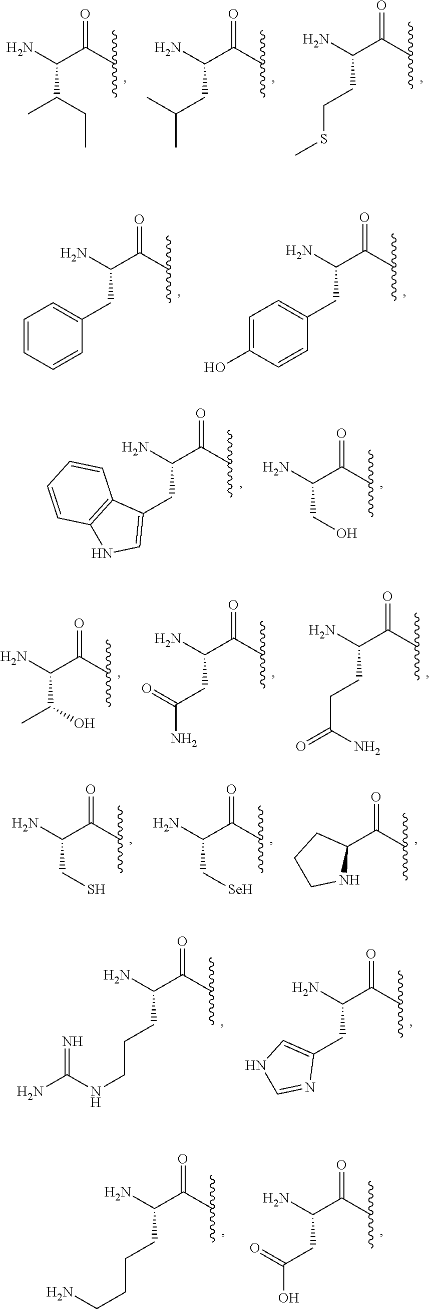

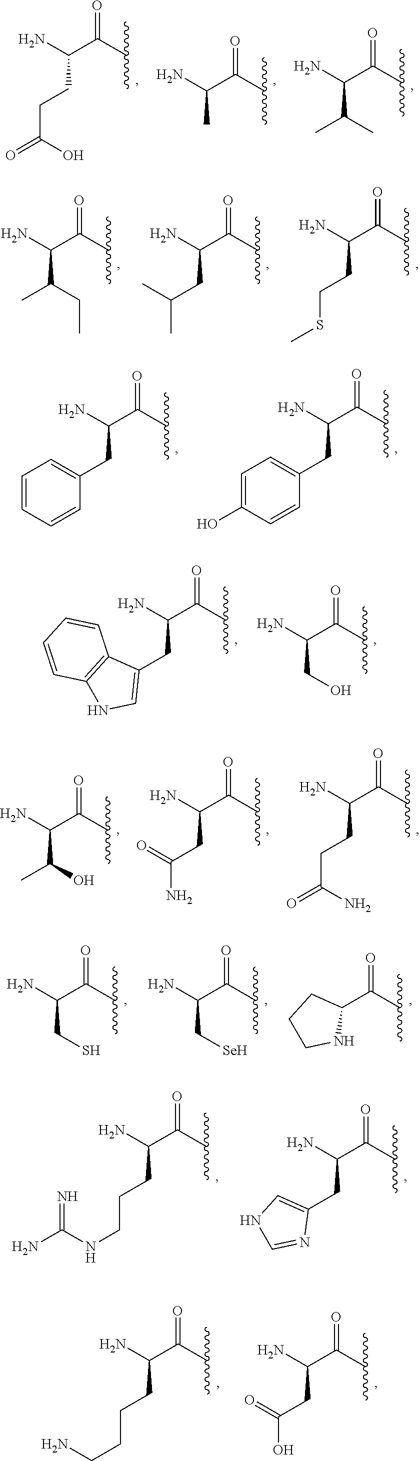

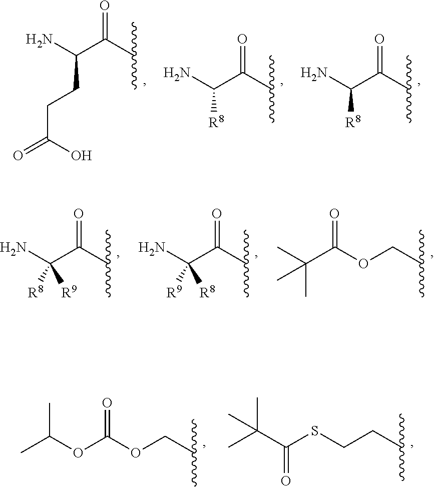

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula XXIII, R¹ is hydrogen,

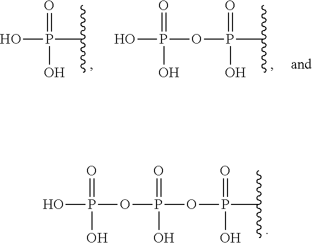 [SMILES: *P(=O)(O)O ; *P(=O)(O)OP(=O)(O)O ; *P(=O)(O)OP(=O)(O)OP(=O)(O)O]

In exemplified embodiments of Formula XXIII, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXIII, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXIII, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXIII, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXIII, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula XXIV,

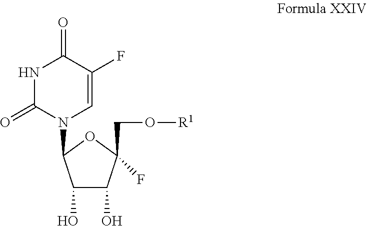

or a pharmaceutical or physiological salt thereof, wherein
R¹ is selected from H,

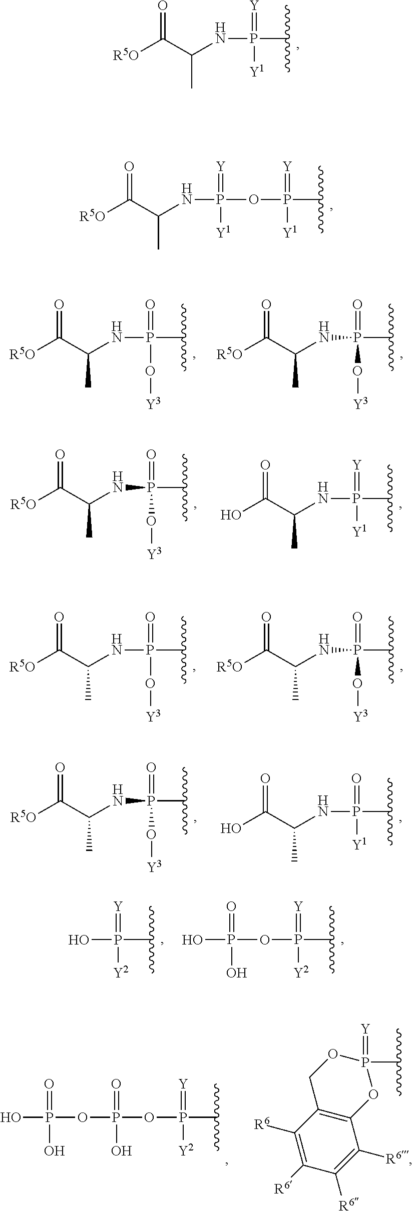

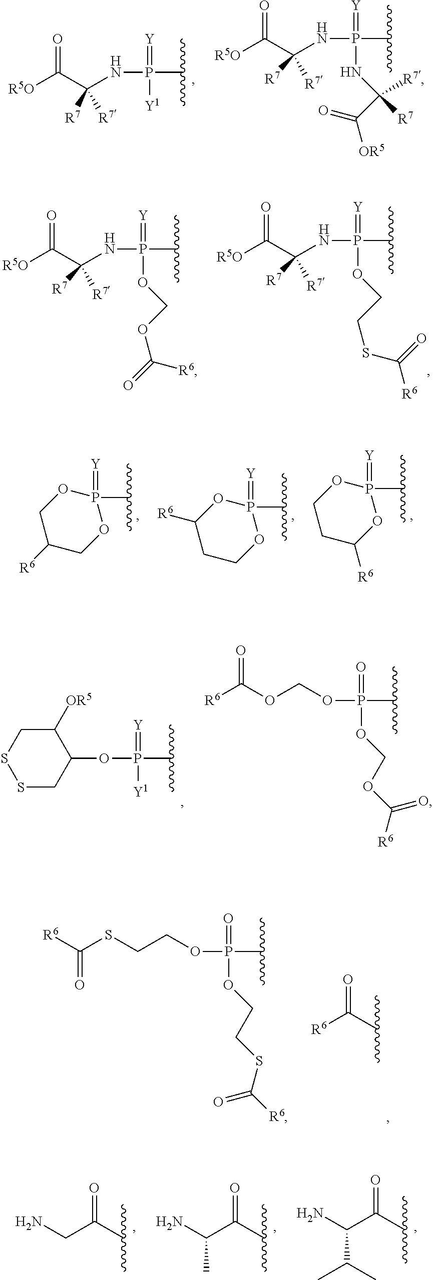

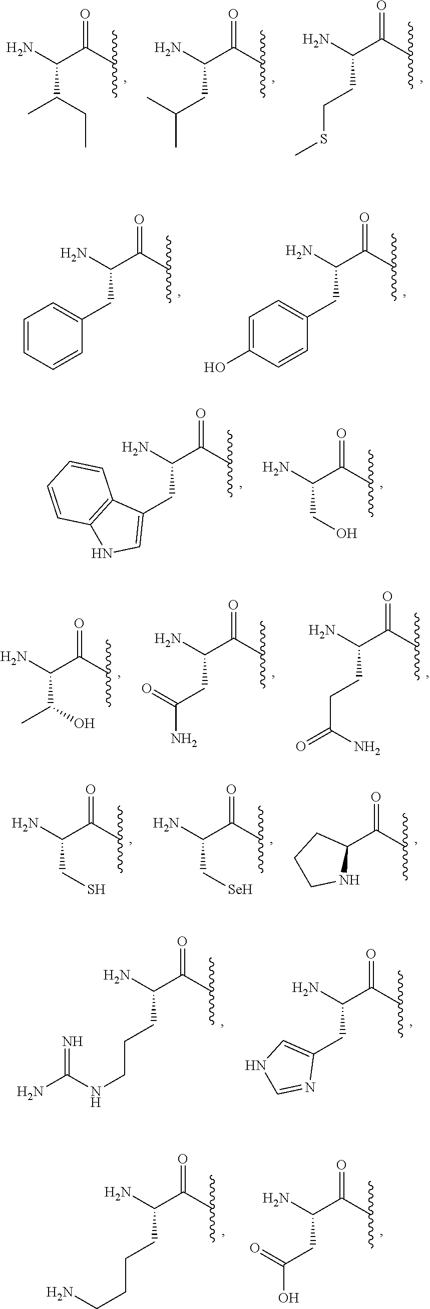

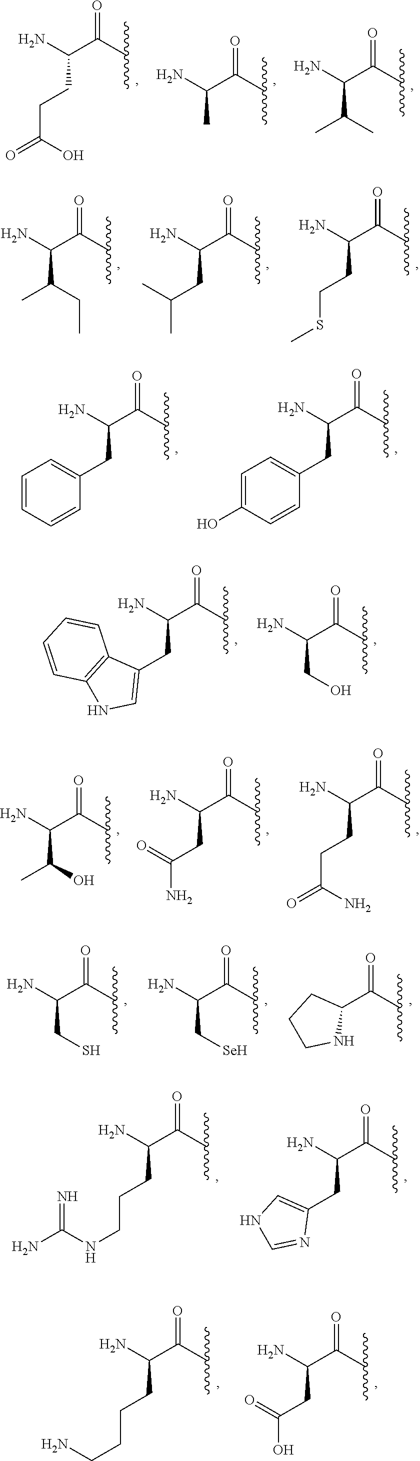

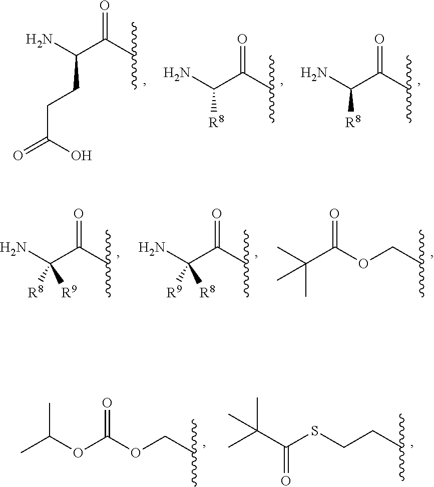

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula XXIV, R¹ is hydrogen,

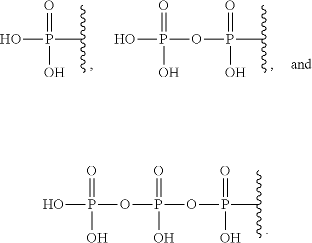 [SMILES: *P(=O)(O)O ; *P(=O)(O)OP(=O)(O)O ; *P(=O)(O)OP(=O)(O)OP(=O)(O)O]

In exemplified embodiments of Formula XXIV, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXIV, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXIV, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXIV, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXIV, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula XXV,

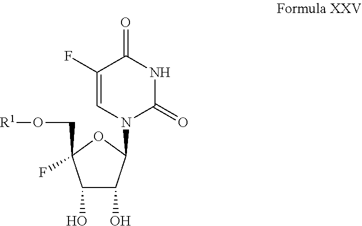

or a pharmaceutical or physiological salt thereof, wherein
R¹ is selected from,

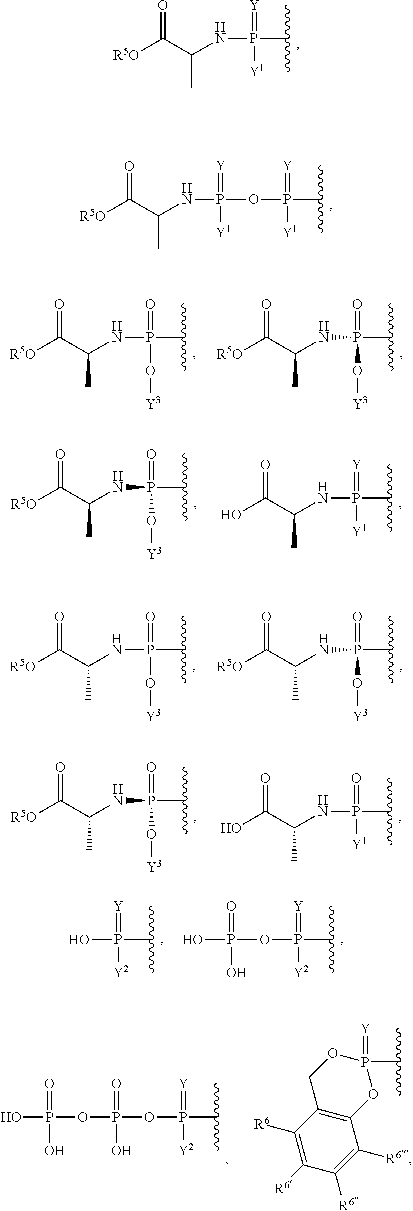

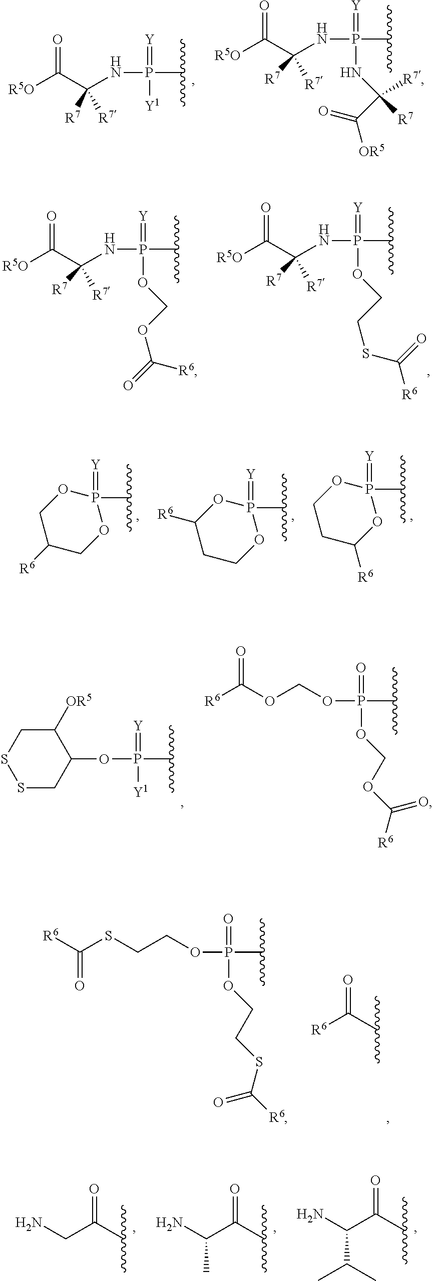

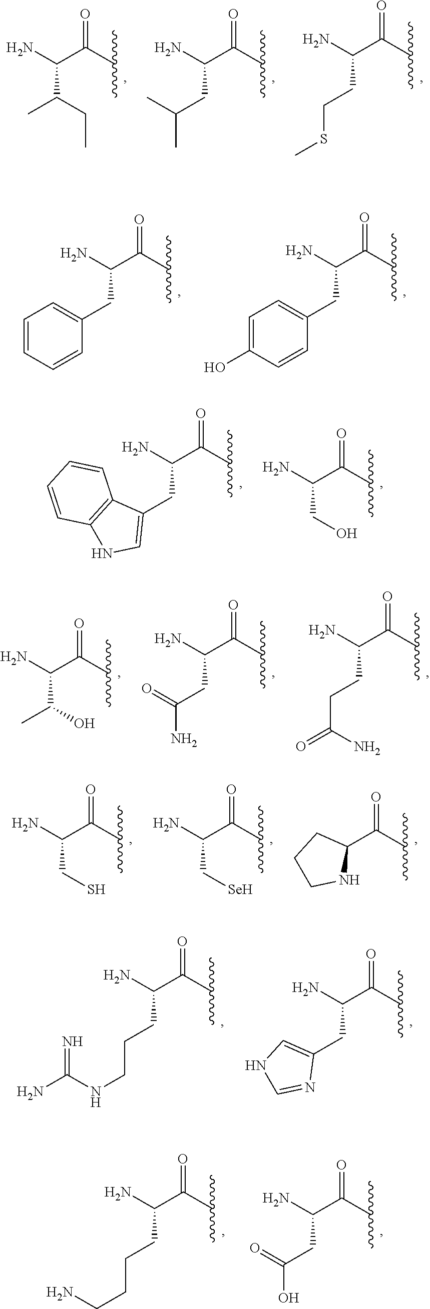

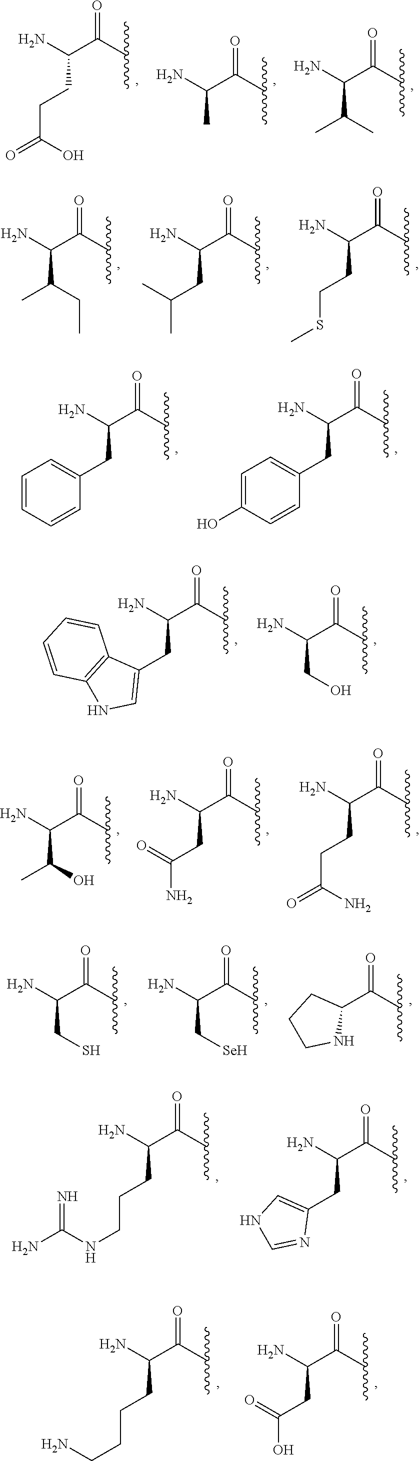

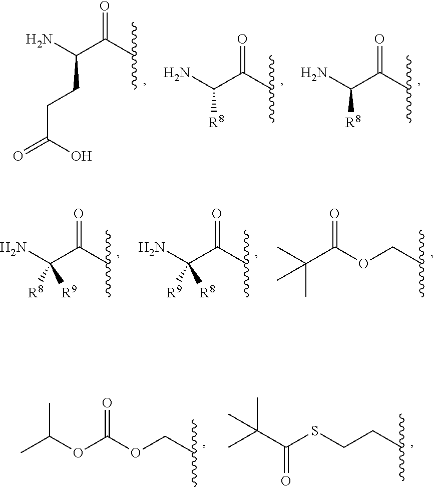

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula XXV, R¹ is hydrogen, OH,

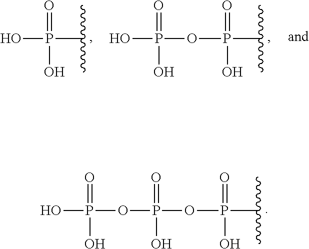

In exemplified embodiments of Formula XXV, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXV, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXV, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXV, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXV, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula XXVI,

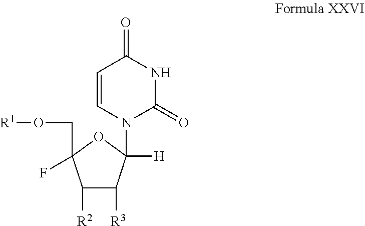

or a pharmaceutical or physiological salt thereof, wherein
R¹ is selected from H

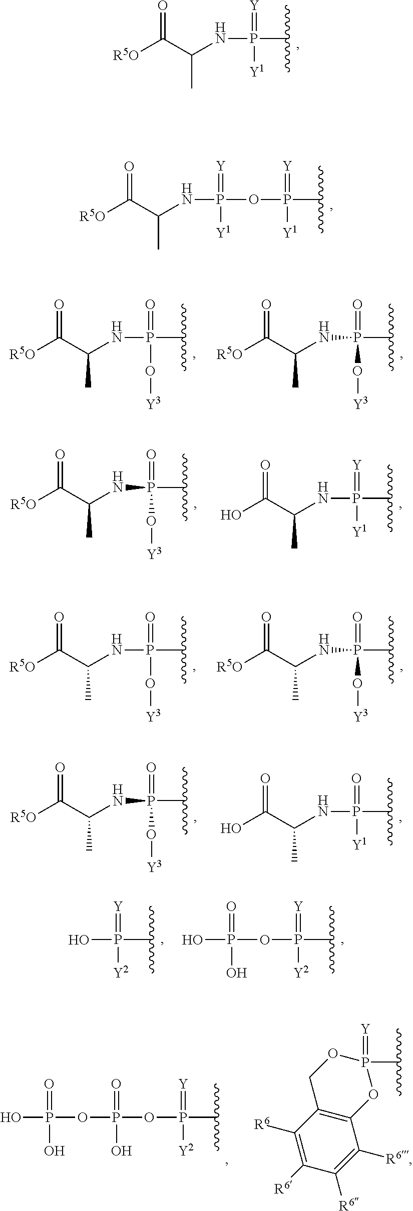

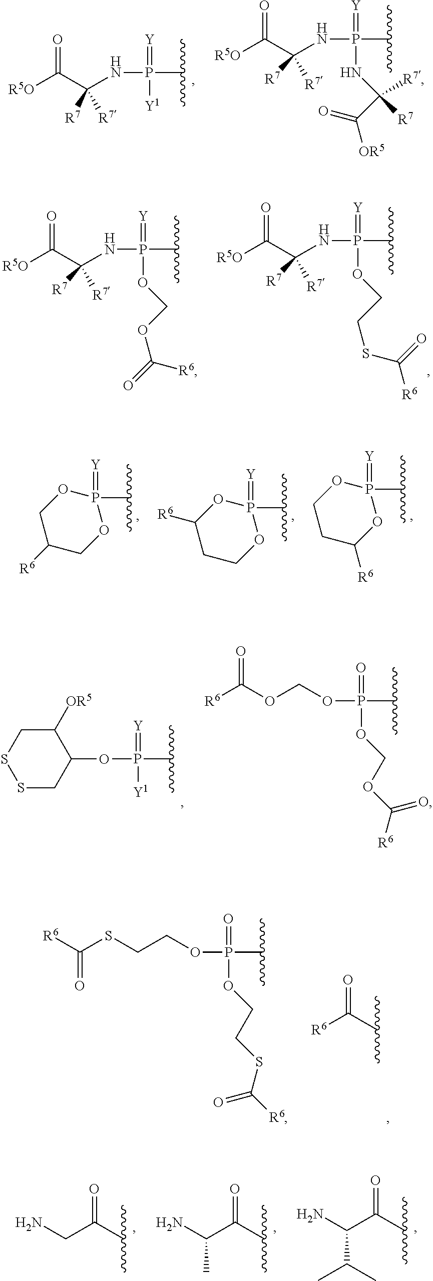

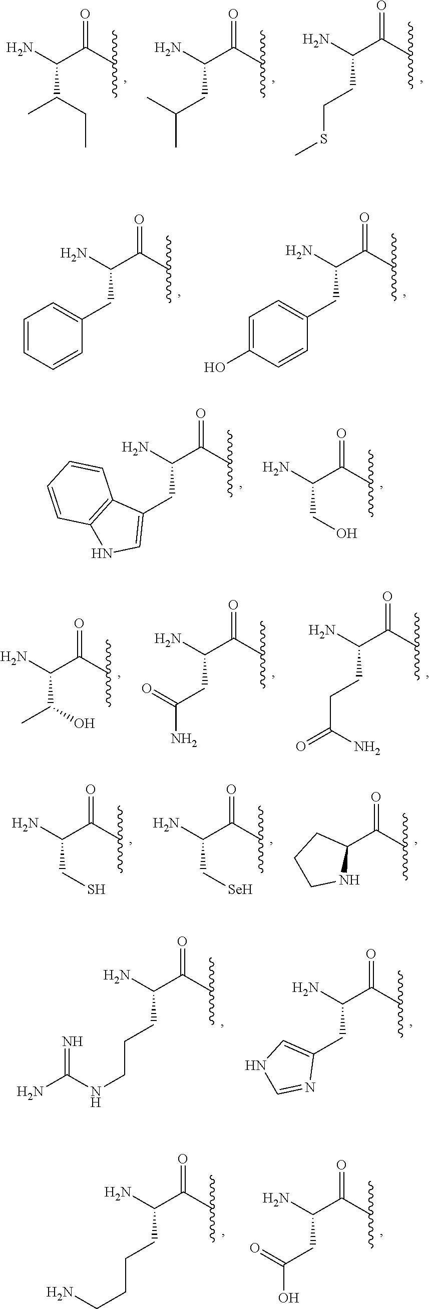

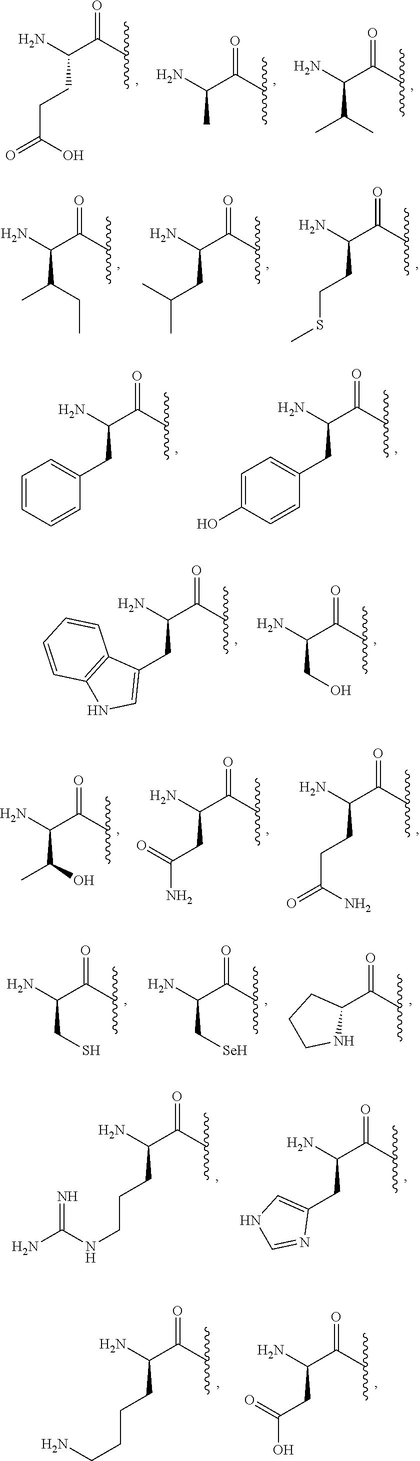

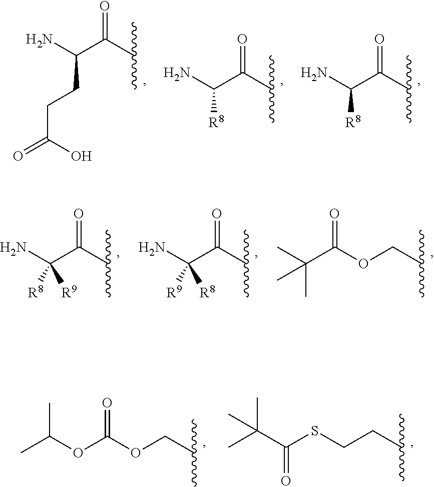

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R² is selected from deuterium, C₂-C₂₂ alkyl, alkenyl, alkynyl, allenyl, thiol, amino, azido, formyl, acyl, cyano, chloro, bromo, iodo, aryl, heteroaryl, heterocyclyl, carbocyclyl, heterocarbocyclyl, sulfinyl, sulfamoyl, or sulfonyl, wherein R² is optionally substituted with one or more, the same or different, R¹⁰;
R³ is selected from deuterium, C₂-C₂₂ alkyl, alkenyl, alkynyl, allenyl, alkoxy, hydroxy, thiol, amino, azido, formyl, acyl, halogen, aryl, heteroaryl, heterocyclyl, carbocyclyl, heterocarbocyclyl, sulfinyl, sulfamoyl, or sulfonyl, wherein R³ and R^{3′} are optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula XXVI, R¹ is hydrogen,

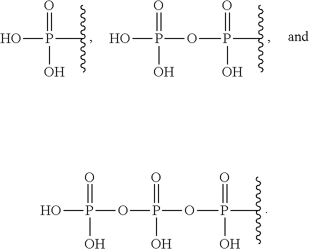

In exemplified embodiments of Formula XXVI, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXVI, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXVI, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXVI, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXVI, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula XXVII,

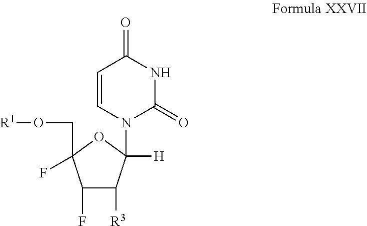

or a pharmaceutical or physiological salt thereof, wherein
R¹ is selected from H,

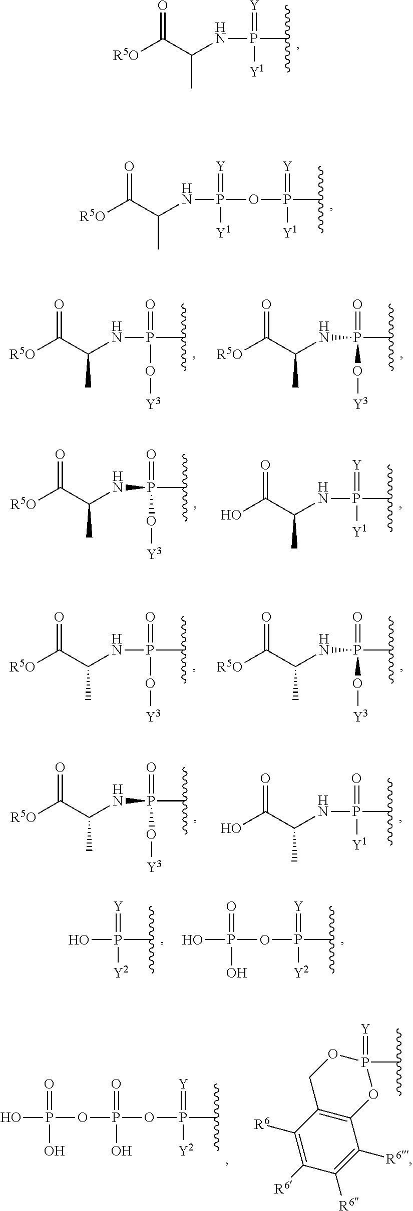

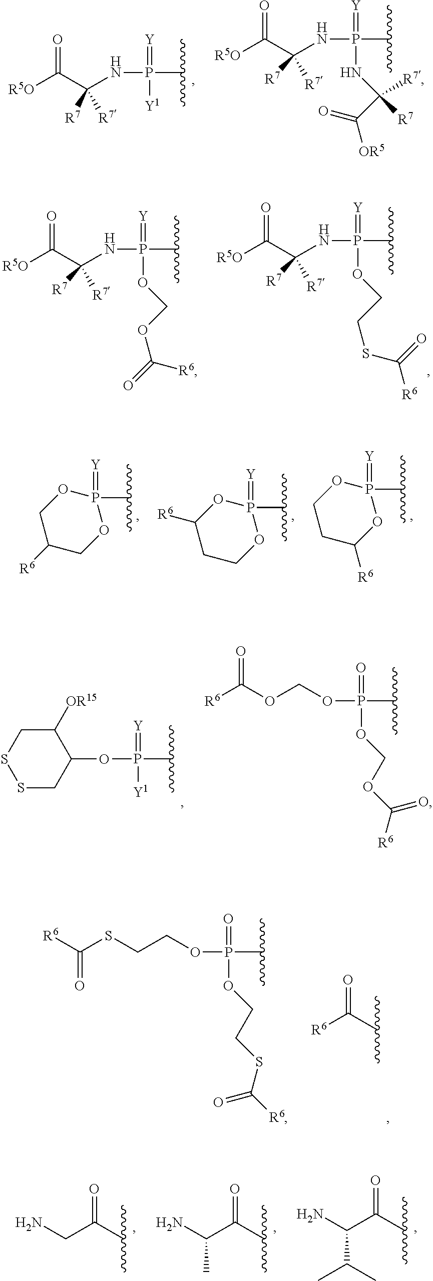

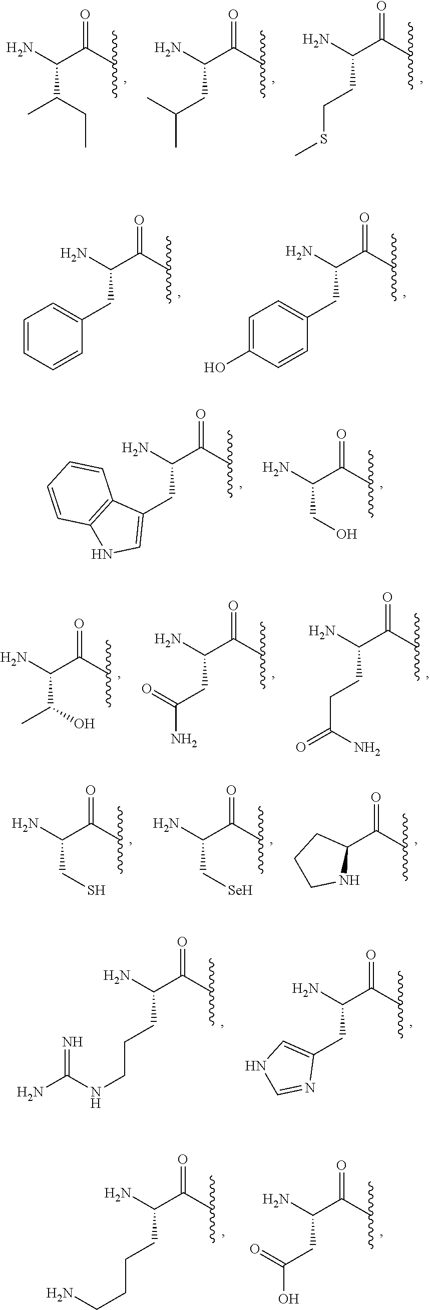

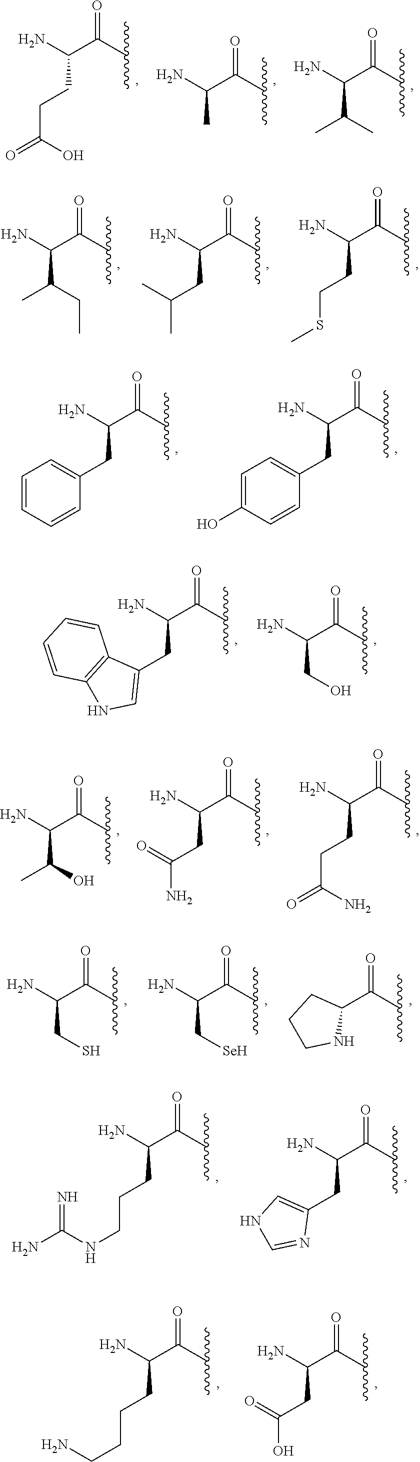

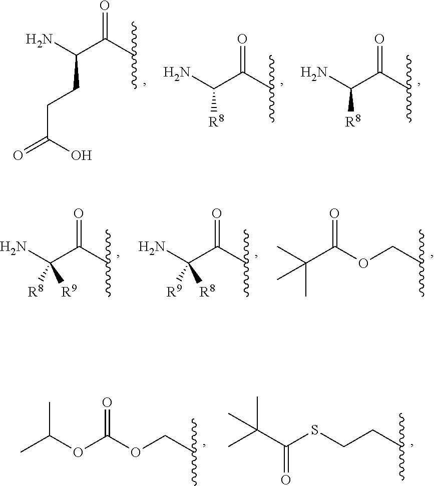

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y² is OH or BH₃⁻M⁺;
Y³ is aryl, heteroaryl, or heterocyclyl, wherein Y³ is optionally substituted with one or more, the same or different, R¹⁰;
R³ is selected from deuterium, C₂-C₂₂ alkyl, alkenyl, alkynyl, allenyl, alkoxy, hydroxy, thiol, amino, azido, formyl, acyl, chloro, bromo, iodo, aryl, heteroaryl, heterocyclyl, carbocyclyl, heterocarbocyclyl, sulfinyl, sulfamoyl, or sulfonyl, wherein R³ and R^{3′} are optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula XXVII, R¹ is hydrogen,

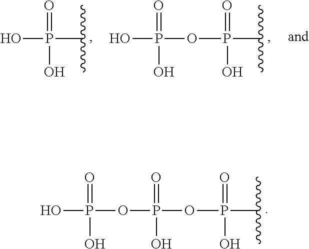

In exemplified embodiments of Formula XXVII, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXVII, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXVII, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXVII, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXVII, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In certain embodiments, the disclosure relates to a compound of Formula XXVIII,

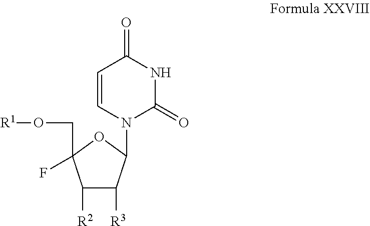

or a pharmaceutical or physiological salt thereof, wherein
R¹ is selected from,

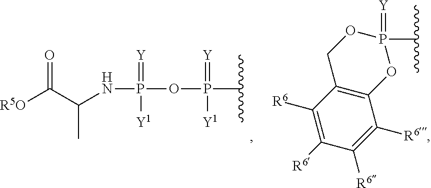

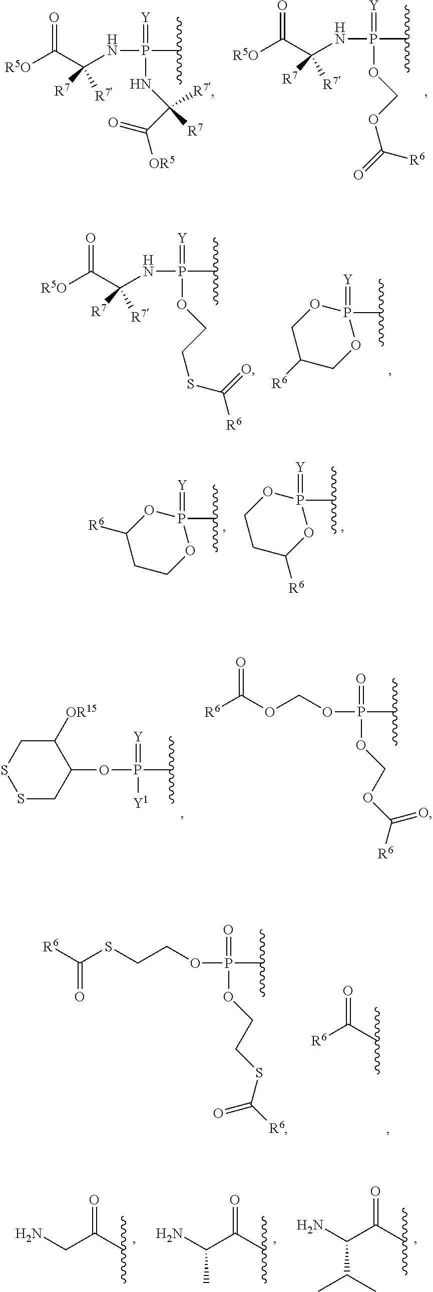

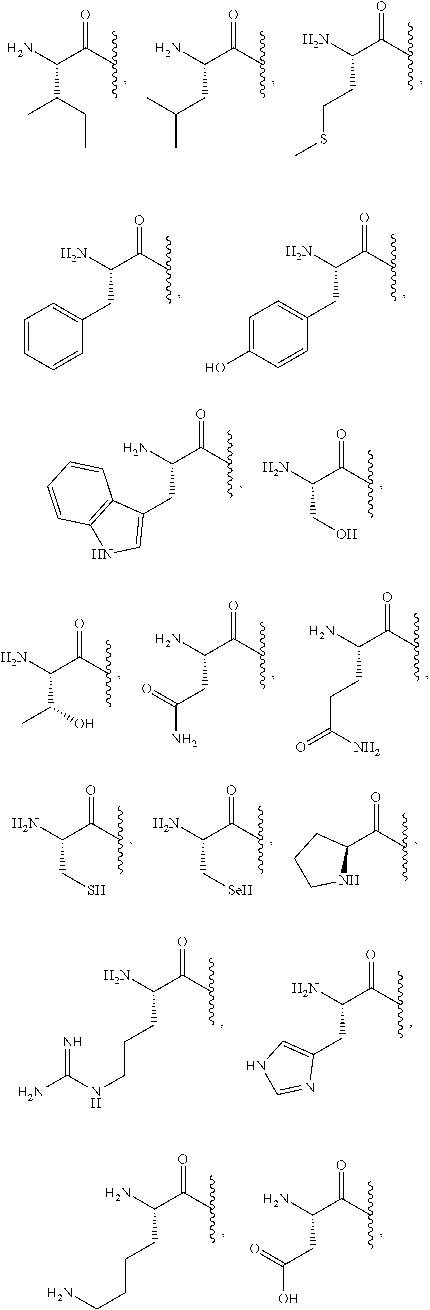

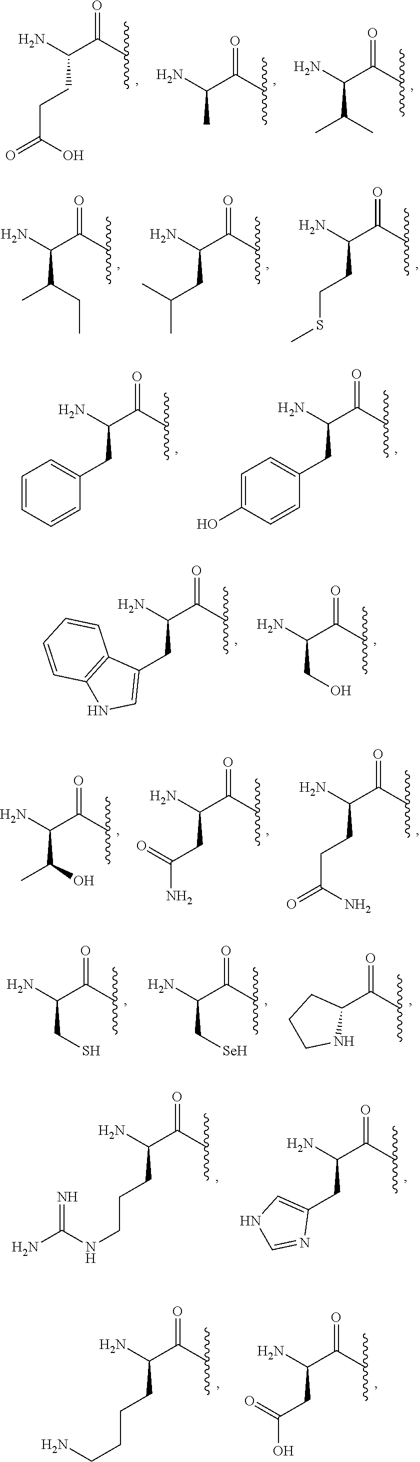

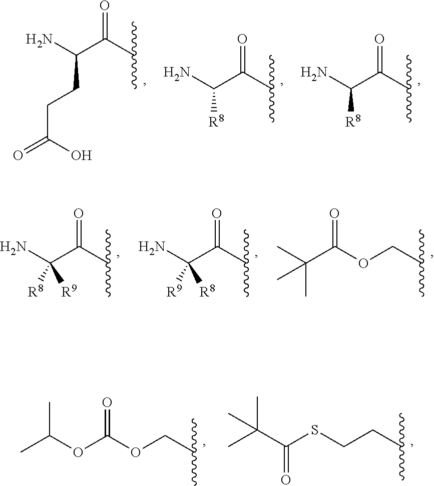

optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thiocarbonate, dithiocarbonates, thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, oxymethoxy amino ester, sulfenyl, sulfinyl, optionally substituted sulfinyl, sulfonyl, optionally substituted sulfonyl, sulfite, optionally substituted sulfite, sulfate, optionally substituted sulfate, sulfonamide, optionally substituted sulfonamide, imidate, hydrazonate, oximyl, imidinyl, imidyl, aminal, hemiaminal, acetal, hemiacetal, carbonimidate, thiocarbonimidate, carbonimidyl, carbamimidate, optionally substituted carbamimidate, carbamimidyl, thioacetal, S-acyl-2-thioethyl, optionally substituted S-acyl-2-thioethyl, (acyloxybenzyl)ether, (acyloxybenzyl)ester, PEG ester, PEG carbonate, bis-(acyloxybenzyl)esters, (acyloxybenzyl)esters, and BAB-esters, wherein R¹ is optionally substituted with one or more, the same or different, R¹⁰;
Y is O or S;
Y¹ is OH, OY³, or BH₃⁻M⁺;
Y³ is aryl, heteroaryl, or heterocyclyl, wherein Y³ is optionally substituted with one or more, the same or different, R¹⁰;
R² is selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R² is optionally substituted with one or more, the same or different, R¹⁰;
R³ is selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R³ is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon they are attached to and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

In exemplified embodiments of Formula XXVIII, R² and R³ are hydrogen, hydroxyl, amino, fluoro, chloro, cyano, methyl, fluoromethyl, methoxy, vinyl, ethynyl, and chloroethynyl.

In exemplified embodiments of Formula XXVIII, R⁵ is lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXVIII, R⁶ is hydrogen, hydroxyl, fluoro, chloro, amino, lipid, methyl, methoxy, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXVIII, R⁷ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXVIII, R⁸ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXVIII, R⁹ is methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplary embodiments, the compound is selected from:

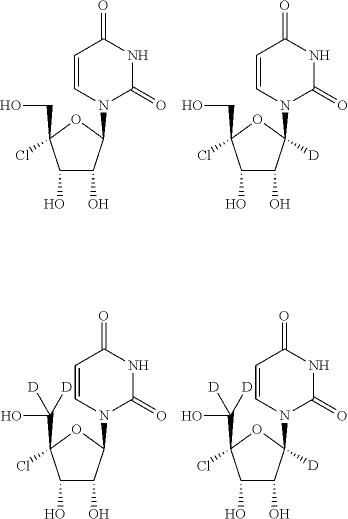

In exemplary embodiments, the compound is selected from:

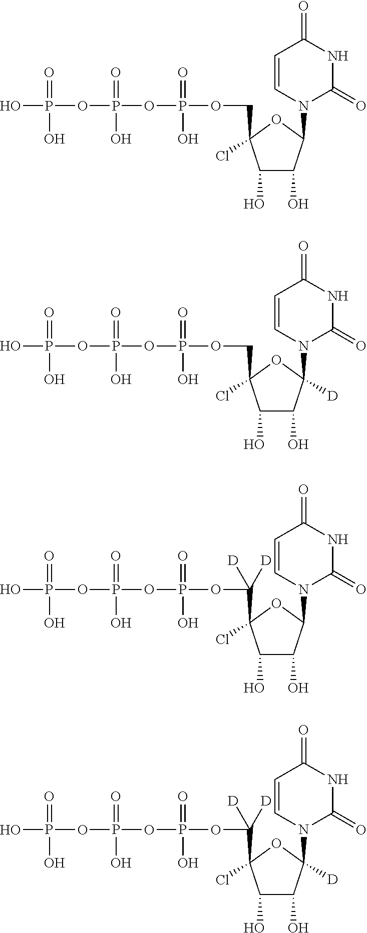

In exemplary embodiments, the compound is selected from:

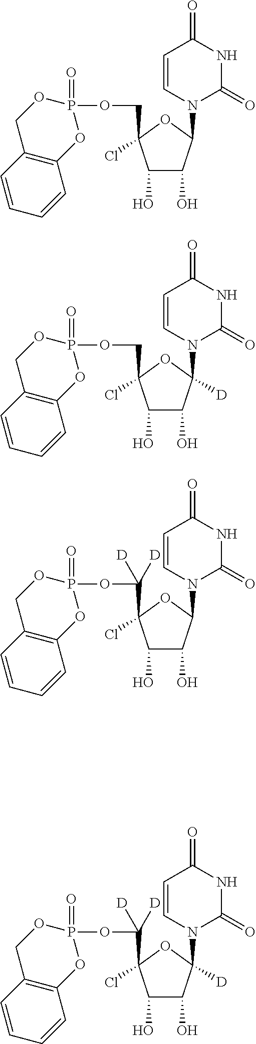

In exemplary embodiments, the compound is selected from:

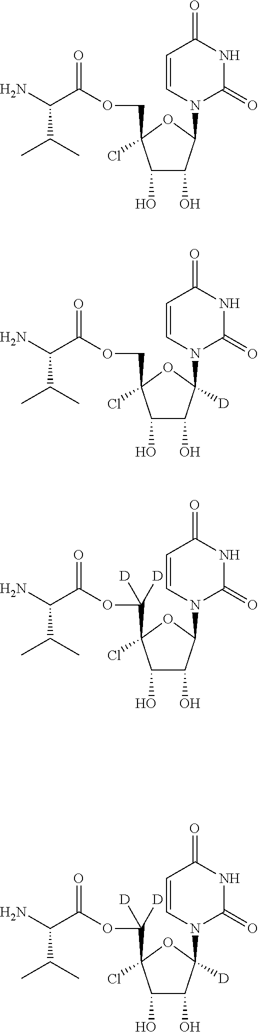 [SMILES: CC(C)[C@H](N)C(=O)OC[C@@]1(Cl)O[C@@H](n2ccc(=O)[nH]c2=O)[C@H](O)[C@@H]1O ; [2H][C@@]1(n2ccc(=O)[nH]c2=O)O[C@](Cl)(COC(=O)[C@@H](N)C(C)C)[C@@H](O)[C@H]1O ; [2H]C([2H])(OC(=O)[C@@H](N)C(C)C)[C@@]1(Cl)O[C@@H](n2ccc(=O)[nH]c2=O)[C@H](O)[C@@H]1O ; [2H]C([2H])(OC(=O)[C@@H](N)C(C)C)[C@@]1(Cl)O[C@@]([2H])(n2ccc(=O)[nH]c2=O)[C@H](O)[C@@H]1O]

In exemplary embodiments, the compound is selected from:

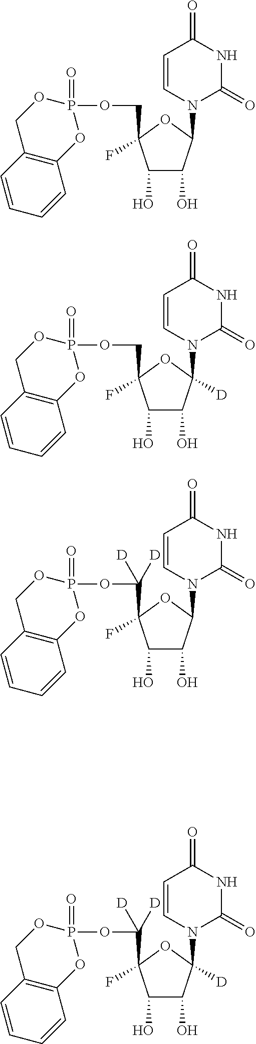

In exemplary embodiments, the compound is selected from:

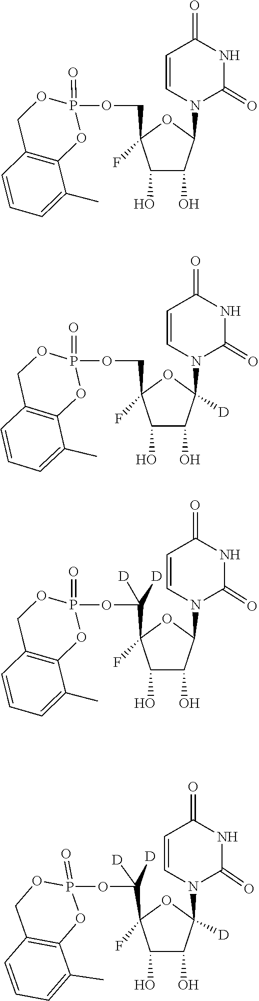

In exemplary embodiments, the compound is selected from:

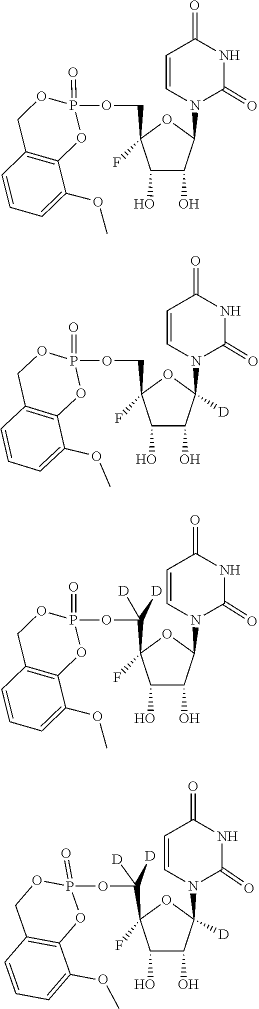

In exemplary embodiments, the compound is selected from:

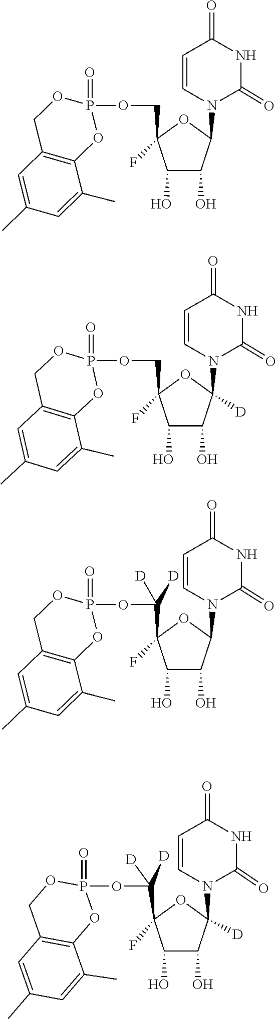

In exemplary embodiments, the compound is selected from:

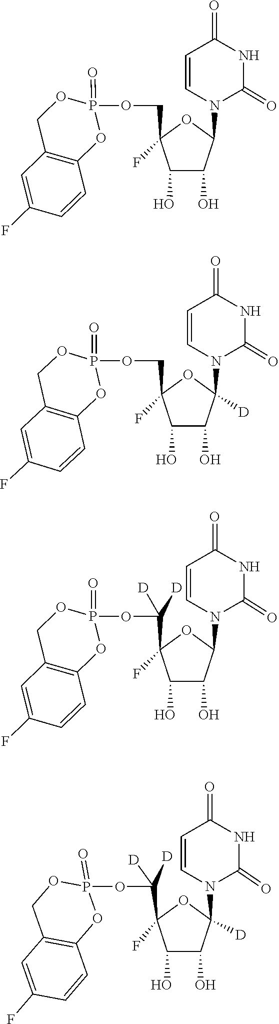

In exemplary embodiments, the compound is selected from:

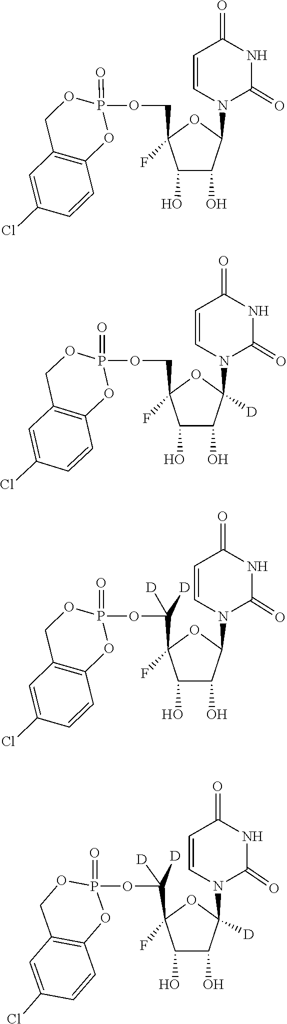

In exemplary embodiments, the compound is selected from:

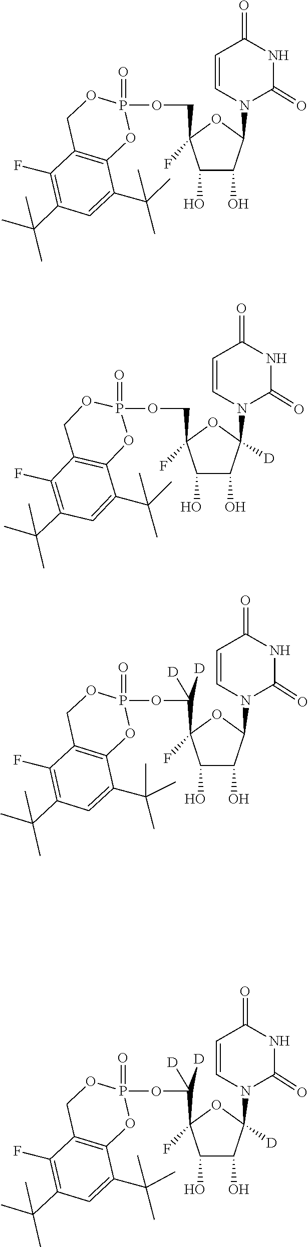

In exemplary embodiments, the compound is selected from:

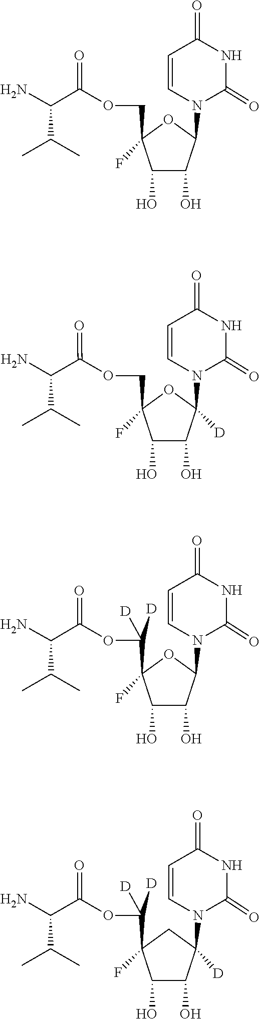

In exemplary embodiments, the compound is selected from:

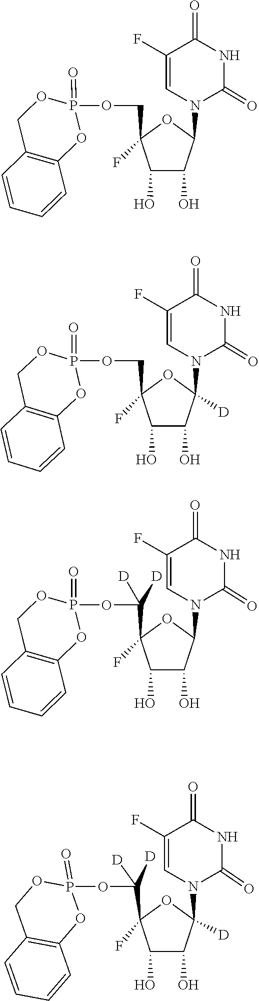

In exemplary embodiments, the compound is selected from:

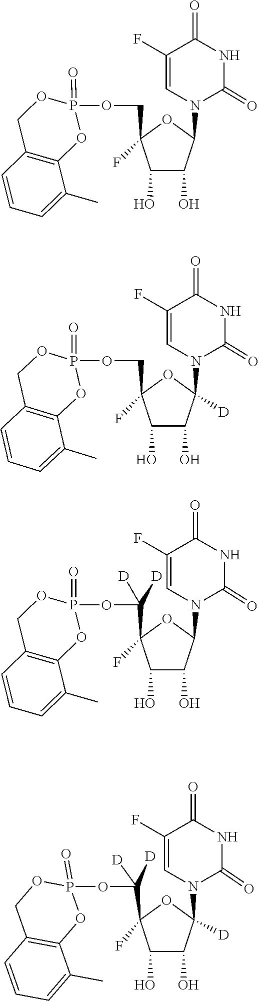

In exemplary embodiments, the compound is selected from:

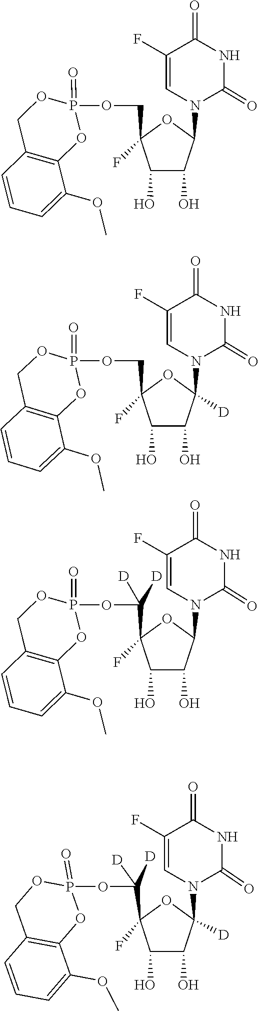

In exemplary embodiments, the compound is selected from:

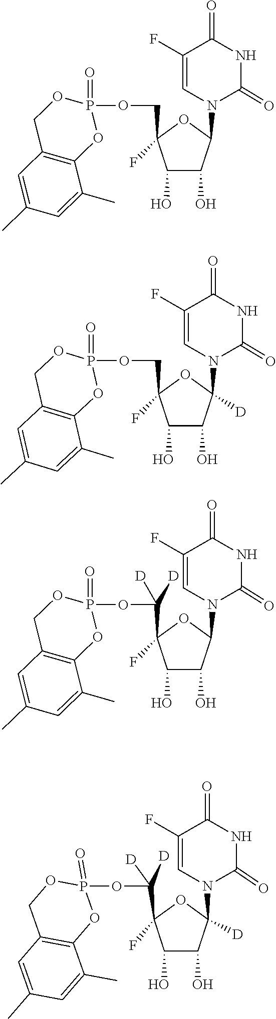

In exemplary embodiments, the compound is selected from:

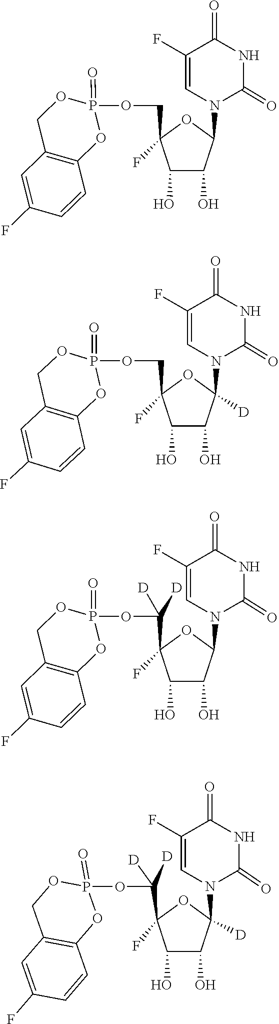

In exemplary embodiments, the compound is selected from:

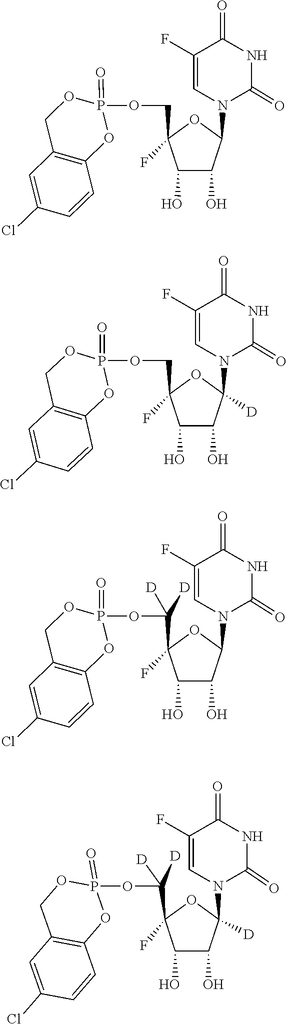

In exemplary embodiments, the compound is selected from:

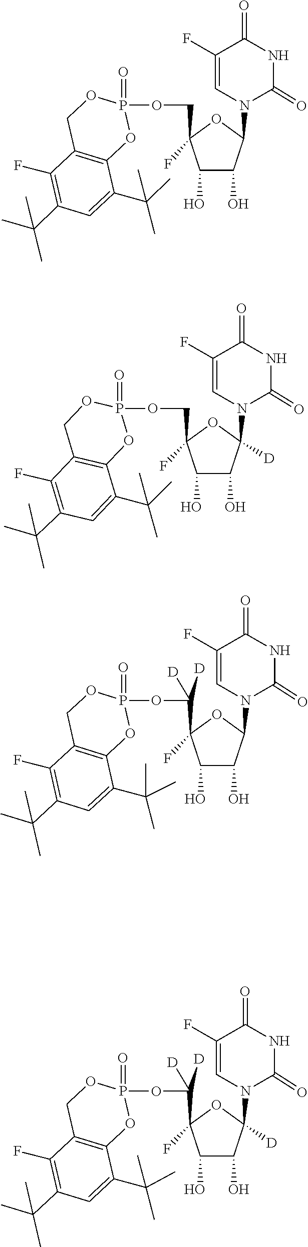

In exemplary embodiments, the compound is selected from:

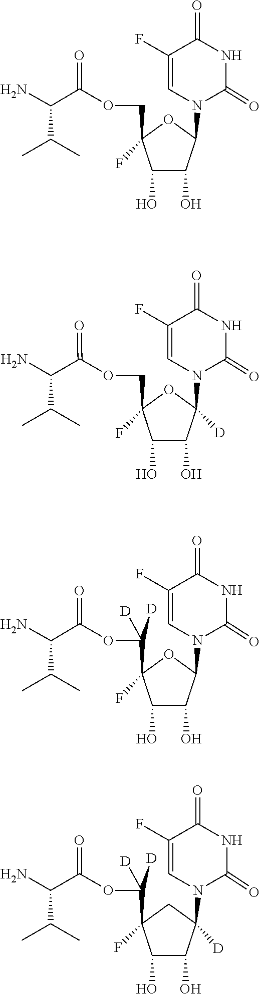

In exemplary embodiments, the compound is selected from:

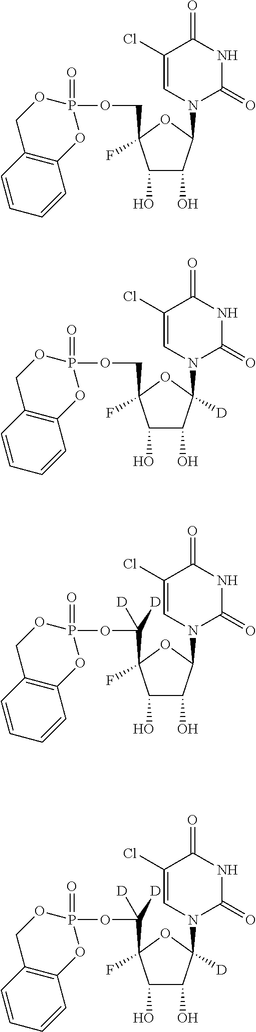

In exemplary embodiments, the compound is selected from:

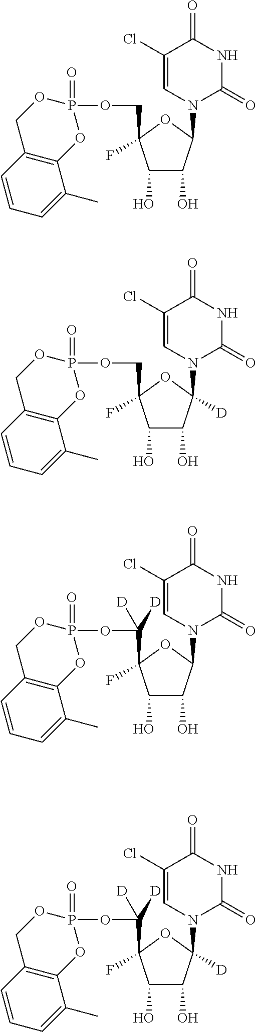

In exemplary embodiments, the compound is selected from:

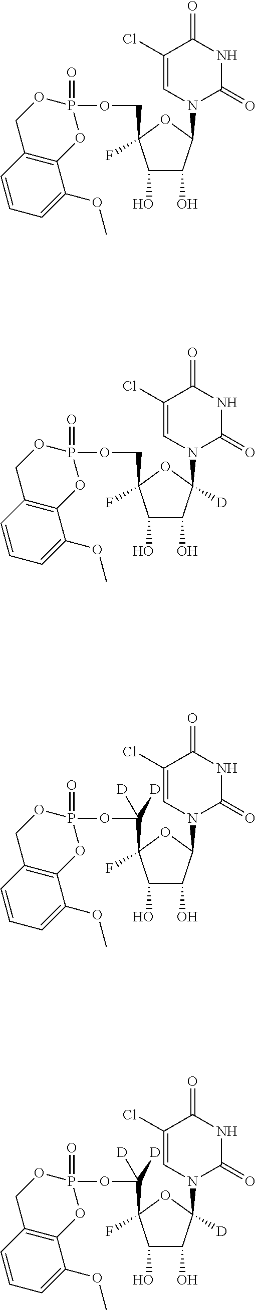

In exemplary embodiments, the compound is selected from:

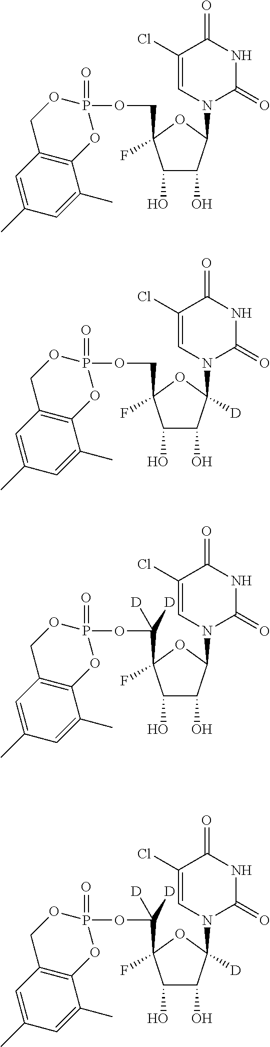

In exemplary embodiments, the compound is selected from:

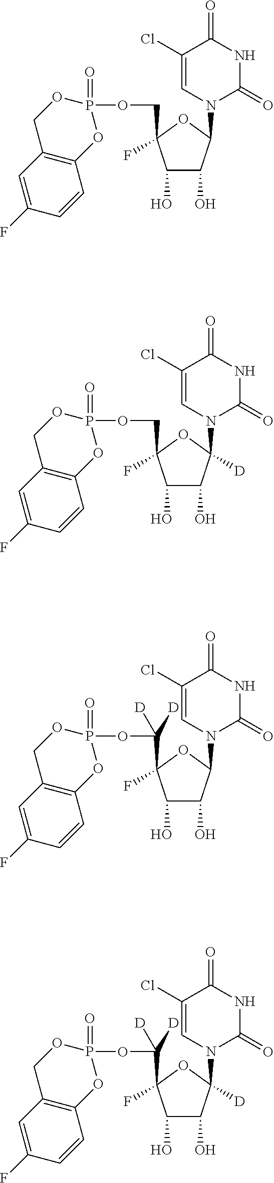

In exemplary embodiments, the compound is selected from:

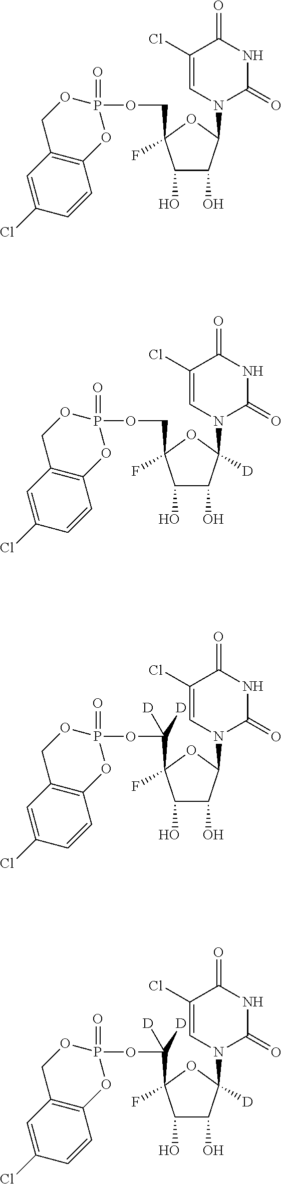

In exemplary embodiments, the compound is selected from:

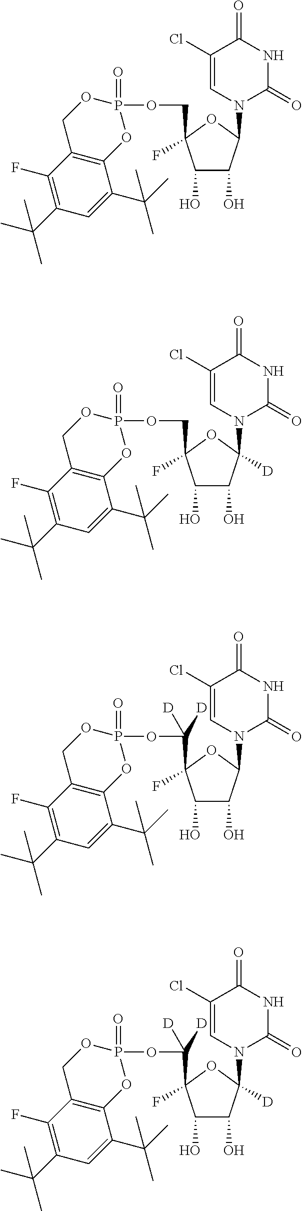

In exemplary embodiments, the compound is selected from:

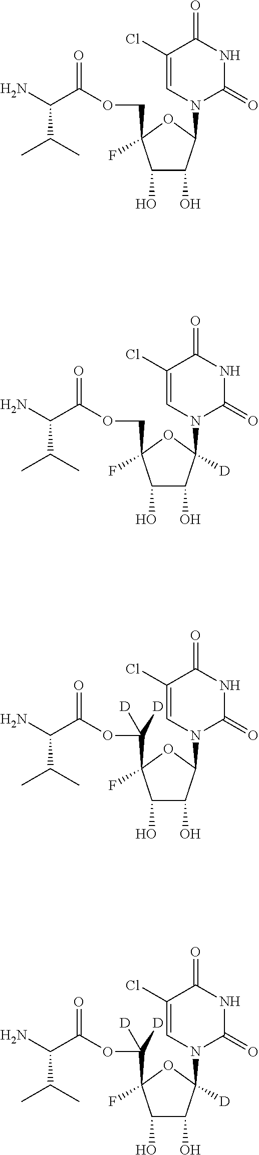

In exemplary embodiments, the compound is selected from:

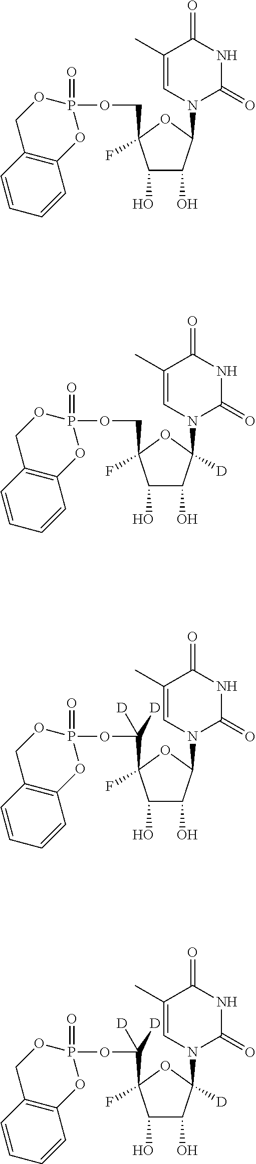

In exemplary embodiments, the compound is selected from:

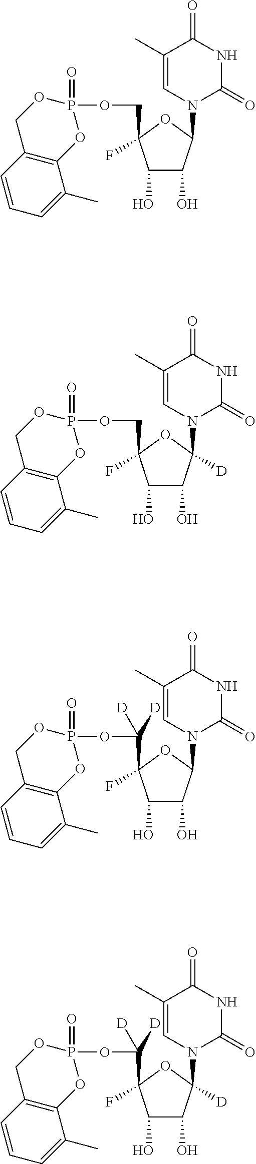

In exemplary embodiments, the compound is selected from:

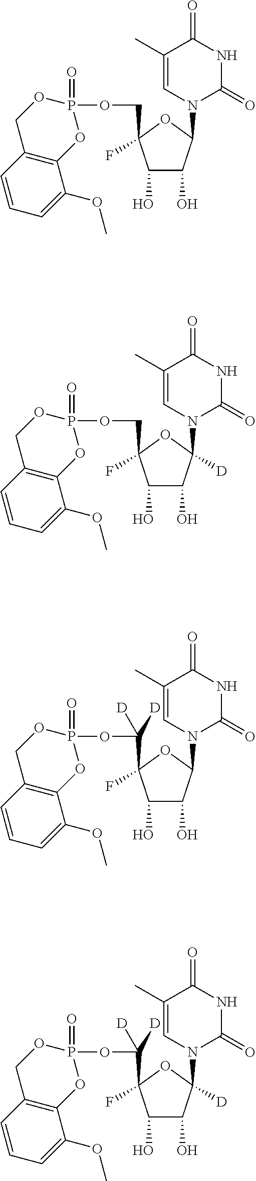

In exemplary embodiments, the compound is selected from:

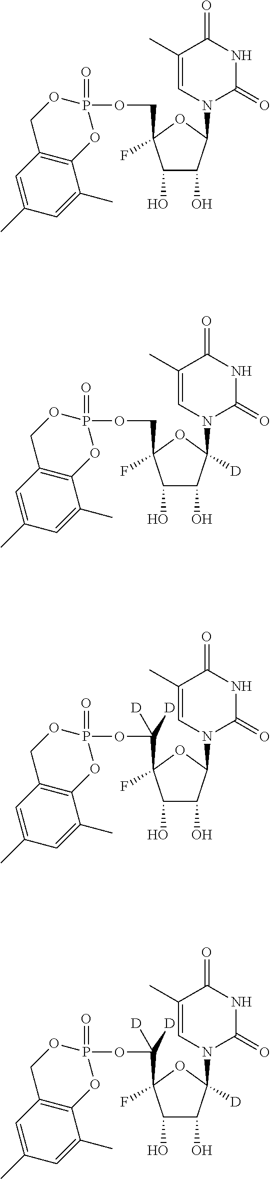

In exemplary embodiments, the compound is selected from:

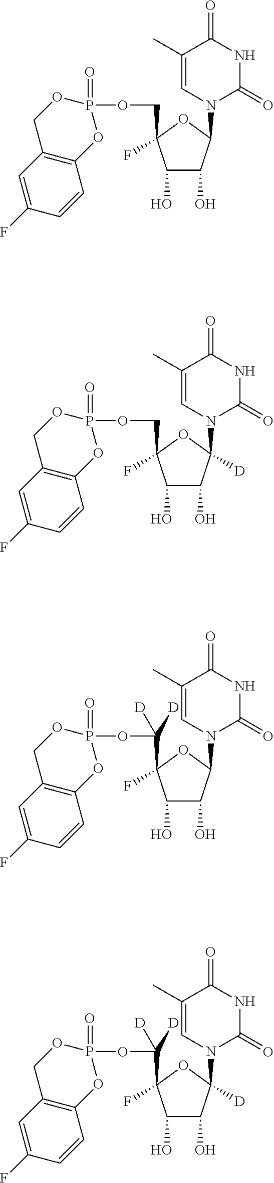

In exemplary embodiments, the compound is selected from:

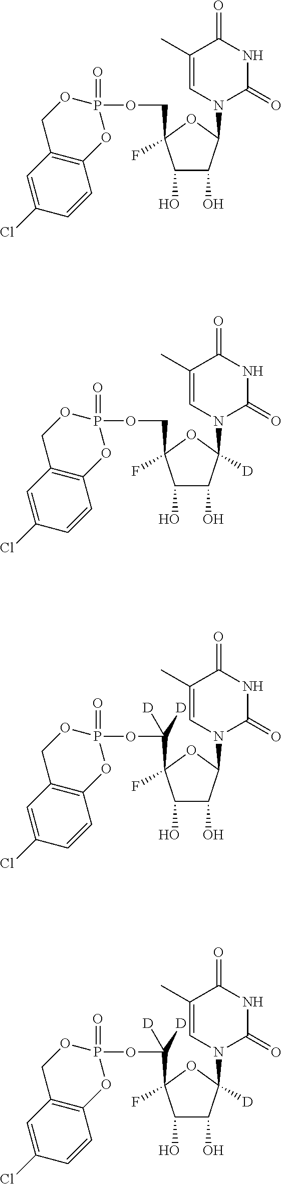

In exemplary embodiments, the compound is selected from:

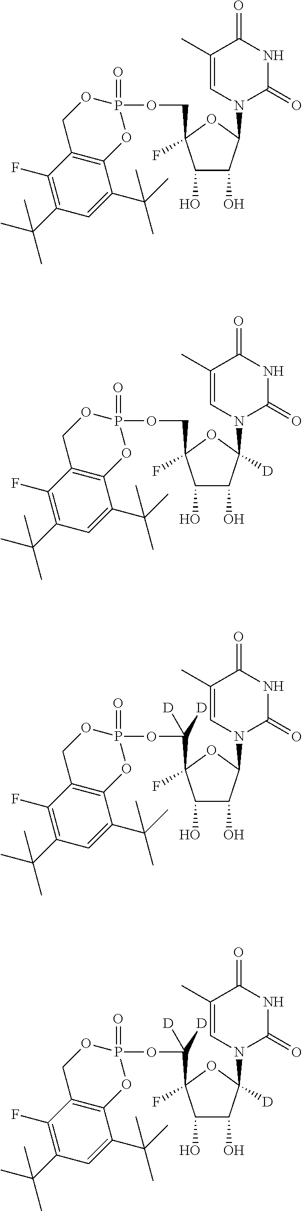

In exemplary embodiments, the compound is selected from:

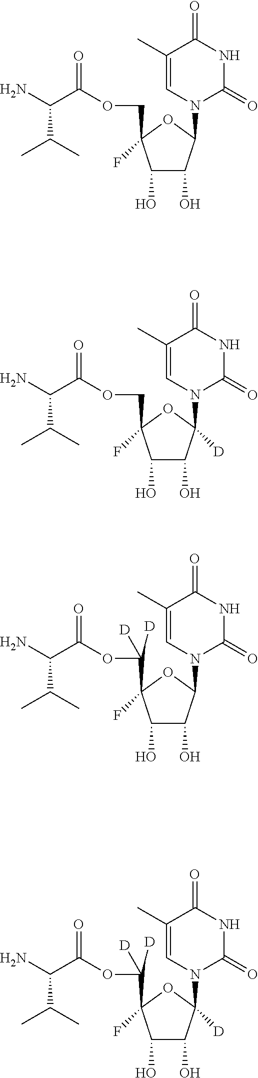 [SMILES: Cc1cn([C@@H]2O[C@](F)(COC(=O)[C@@H](N)C(C)C)[C@@H](O)[C@H]2O)c(=O)[nH]c1=O ; [2H][C@@]1(n2cc(C)c(=O)[nH]c2=O)O[C@](F)(COC(=O)[C@@H](N)C(C)C)[C@@H](O)[C@H]1O ; [2H]C([2H])(OC(=O)[C@@H](N)C(C)C)[C@@]1(F)O[C@@H](n2cc(C)c(=O)[nH]c2=O)[C@H](O)[C@@H]1O ; [2H]C([2H])(OC(=O)[C@@H](N)C(C)C)[C@@]1(F)O[C@@]([2H])(n2cc(C)c(=O)[nH]c2=O)[C@H](O)[C@@H]1O]

In exemplary embodiments, the compound is selected from:

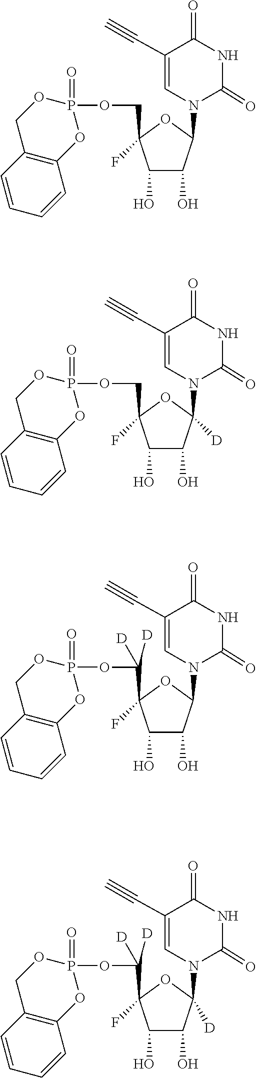 [SMILES: C#Cc1cn([C@@H]2O[C@](F)(COP3(=O)OCc4ccccc4O3)[C@@H](O)[C@H]2O)c(=O)[nH]c1=O ; [2H][C@@]1(n2cc(C#C)c(=O)[nH]c2=O)O[C@](F)(COP2(=O)OCc3ccccc3O2)[C@@H](O)[C@H]1O ; [2H]C([2H])(OP1(=O)OCc2ccccc2O1)[C@@]1(F)O[C@@H](n2cc(C#C)c(=O)[nH]c2=O)[C@H](O)[C@@H]1O ; [2H]C([2H])(OP1(=O)OCc2ccccc2O1)[C@@]1(F)O[C@@]([2H])(n2cc(C#C)c(=O)[nH]c2=O)[C@H](O)[C@@H]1O]

In exemplary embodiments, the compound is selected from:

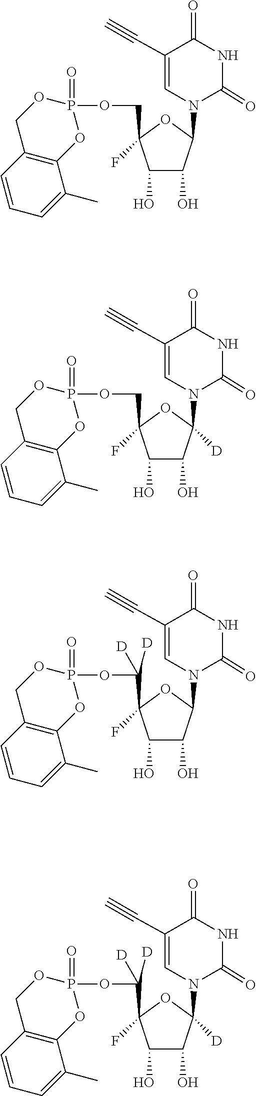 [SMILES: C#Cc1cn([C@@H]2O[C@](F)(COP3(=O)OCc4cccc(C)c4O3)[C@@H](O)[C@H]2O)c(=O)[nH]c1=O ; [2H][C@@]1(n2cc(C#C)c(=O)[nH]c2=O)O[C@](F)(COP2(=O)OCc3cccc(C)c3O2)[C@@H](O)[C@H]1O ; [2H]C([2H])(OP1(=O)OCc2cccc(C)c2O1)[C@@]1(F)O[C@@H](n2cc(C#C)c(=O)[nH]c2=O)[C@H](O)[C@@H]1O ; [2H]C([2H])(OP1(=O)OCc2cccc(C)c2O1)[C@@]1(F)O[C@@]([2H])(n2cc(C#C)c(=O)[nH]c2=O)[C@H](O)[C@@H]1O]

In exemplary embodiments, the compound is selected from:

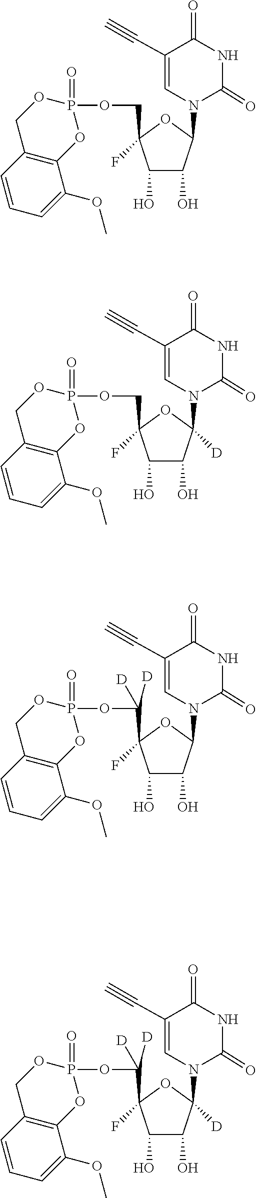 [SMILES: C#Cc1cn([C@@H]2O[C@](F)(COP3(=O)OCc4cccc(OC)c4O3)[C@@H](O)[C@H]2O)c(=O)[nH]c1=O ; [2H][C@@]1(n2cc(C#C)c(=O)[nH]c2=O)O[C@](F)(COP2(=O)OCc3cccc(OC)c3O2)[C@@H](O)[C@H]1O ; [2H]C([2H])(OP1(=O)OCc2cccc(OC)c2O1)[C@@]1(F)O[C@@H](n2cc(C#C)c(=O)[nH]c2=O)[C@H](O)[C@@H]1O ; [2H]C([2H])(OP1(=O)OCc2cccc(OC)c2O1)[C@@]1(F)O[C@@]([2H])(n2cc(C#C)c(=O)[nH]c2=O)[C@H](O)[C@@H]1O]

In exemplary embodiments, the compound is selected from:

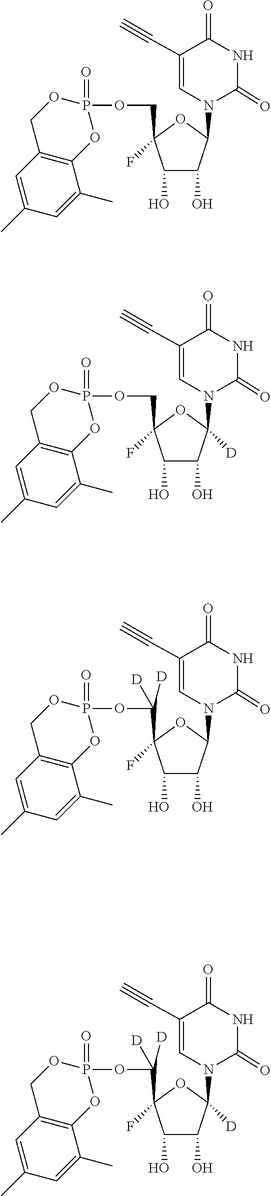 [SMILES: C#Cc1cn([C@@H]2O[C@](F)(COP3(=O)OCc4cc(C)cc(C)c4O3)[C@@H](O)[C@H]2O)c(=O)[nH]c1=O ; [2H][C@@]1(n2cc(C#C)c(=O)[nH]c2=O)O[C@](F)(COP2(=O)OCc3cc(C)cc(C)c3O2)[C@@H](O)[C@H]1O ; [2H]C([2H])(OP1(=O)OCc2cc(C)cc(C)c2O1)[C@@]1(F)O[C@@H](n2cc(C#C)c(=O)[nH]c2=O)[C@H](O)[C@@H]1O ; [2H]C([2H])(OP1(=O)OCc2cc(C)cc(C)c2O1)[C@@]1(F)O[C@@]([2H])(n2cc(C#C)c(=O)[nH]c2=O)[C@H](O)[C@@H]1O]

In exemplary embodiments, the compound is selected from:

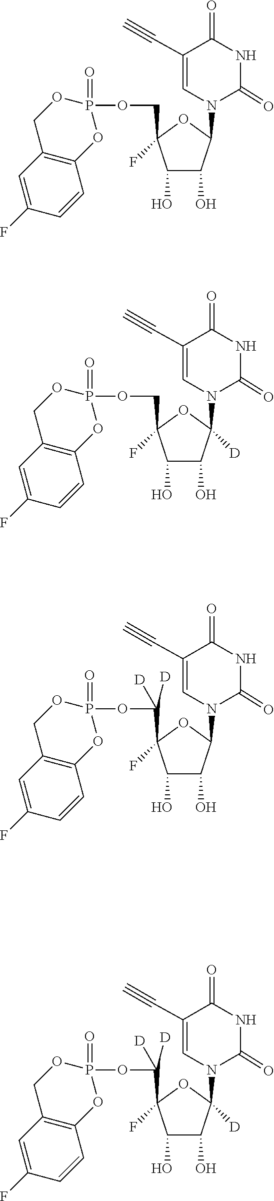 [SMILES: C#Cc1cn([C@@H]2O[C@](F)(COP3(=O)OCc4cc(F)ccc4O3)[C@@H](O)[C@H]2O)c(=O)[nH]c1=O ; [2H][C@@]1(n2cc(C#C)c(=O)[nH]c2=O)O[C@](F)(COP2(=O)OCc3cc(F)ccc3O2)[C@@H](O)[C@H]1O ; [2H]C([2H])(OP1(=O)OCc2cc(F)ccc2O1)[C@@]1(F)O[C@@H](n2cc(C#C)c(=O)[nH]c2=O)[C@H](O)[C@@H]1O ; [2H]C([2H])(OP1(=O)OCc2cc(F)ccc2O1)[C@@]1(F)O[C@@]([2H])(n2cc(C#C)c(=O)[nH]c2=O)[C@H](O)[C@@H]1O]

In exemplary embodiments, the compound is selected from:

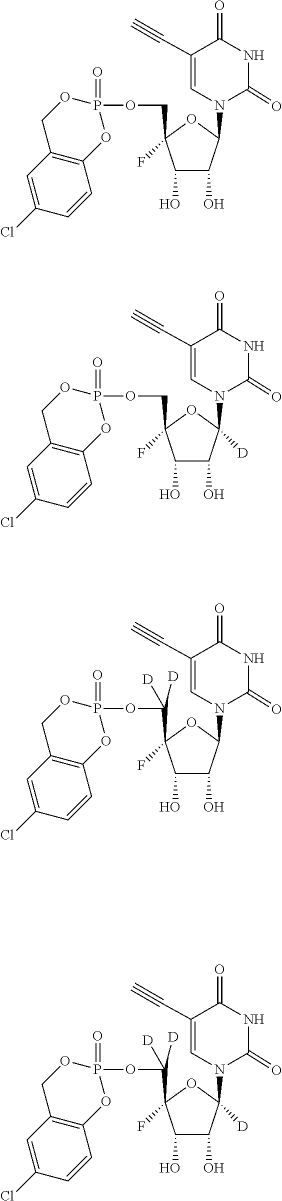 [SMILES: C#Cc1cn([C@@H]2O[C@](F)(COP3(=O)OCc4cc(Cl)ccc4O3)[C@@H](O)[C@H]2O)c(=O)[nH]c1=O ; [2H][C@@]1(n2cc(C#C)c(=O)[nH]c2=O)O[C@](F)(COP2(=O)OCc3cc(Cl)ccc3O2)[C@@H](O)[C@H]1O ; [2H]C([2H])(OP1(=O)OCc2cc(Cl)ccc2O1)[C@@]1(F)O[C@@H](n2cc(C#C)c(=O)[nH]c2=O)[C@H](O)[C@@H]1O ; [2H]C([2H])(OP1(=O)OCc2cc(Cl)ccc2O1)[C@@]1(F)O[C@@]([2H])(n2cc(C#C)c(=O)[nH]c2=O)[C@H](O)[C@@H]1O]

In exemplary embodiments, the compound is selected from:

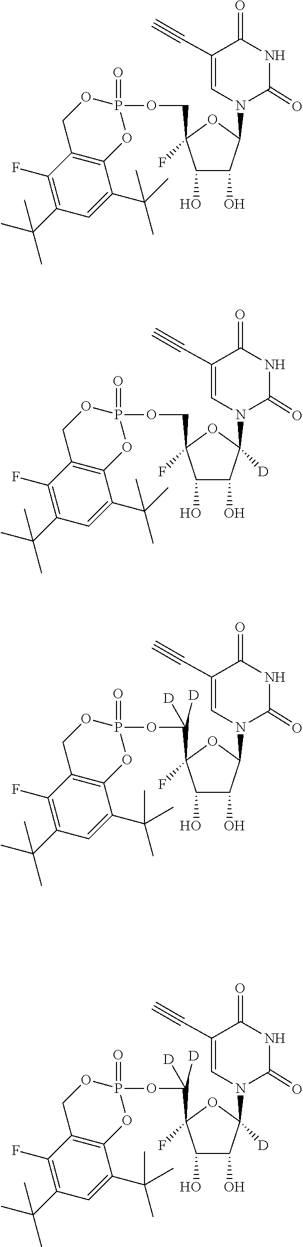 [SMILES: C#Cc1cn([C@@H]2O[C@](F)(COP3(=O)OCc4c(F)c(C(C)(C)C)cc(C(C)(C)C)c4O3)[C@@H](O)[C@H]2O)c(=O)[nH]c1=O ; [2H][C@@]1(n2cc(C#C)c(=O)[nH]c2=O)O[C@](F)(COP2(=O)OCc3c(F)c(C(C)(C)C)cc(C(C)(C)C)c3O2)[C@@H](O)[C@H]1O ; [2H]C([2H])(OP1(=O)OCc2c(F)c(C(C)(C)C)cc(C(C)(C)C)c2O1)[C@@]1(F)O[C@@H](n2cc(C#C)c(=O)[nH]c2=O)[C@H](O)[C@@H]1O ; [2H]C([2H])(OP1(=O)OCc2c(F)c(C(C)(C)C)cc(C(C)(C)C)c2O1)[C@@]1(F)O[C@@]([2H])(n2cc(C#C)c(=O)[nH]c2=O)[C@H](O)[C@@H]1O]

In exemplary embodiments, the compound is selected from:

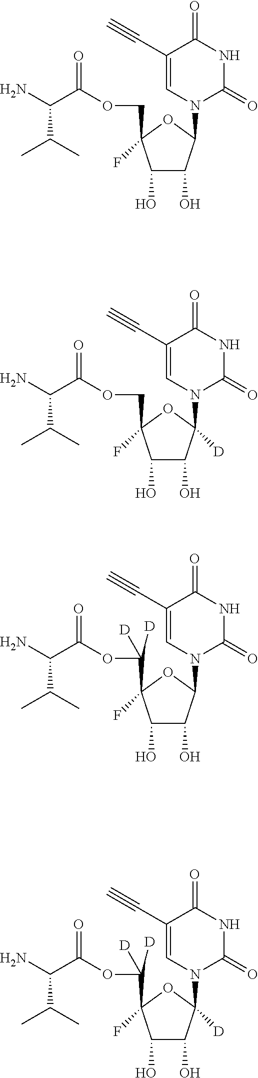 [SMILES: C#Cc1cn([C@@H]2O[C@](F)(COC(=O)[C@@H](N)C(C)C)[C@@H](O)[C@H]2O)c(=O)[nH]c1=O ; [2H][C@@]1(n2cc(C#C)c(=O)[nH]c2=O)O[C@](F)(COC(=O)[C@@H](N)C(C)C)[C@@H](O)[C@H]1O ; [2H]C([2H])(OC(=O)[C@@H](N)C(C)C)[C@@]1(F)O[C@@H](n2cc(C#C)c(=O)[nH]c2=O)[C@H](O)[C@@H]1O ; [2H]C([2H])(OC(=O)[C@@H](N)C(C)C)[C@@]1(F)O[C@@]([2H])(n2cc(C#C)c(=O)[nH]c2=O)[C@H](O)[C@@H]1O]

In exemplary embodiments, the compound is selected from:

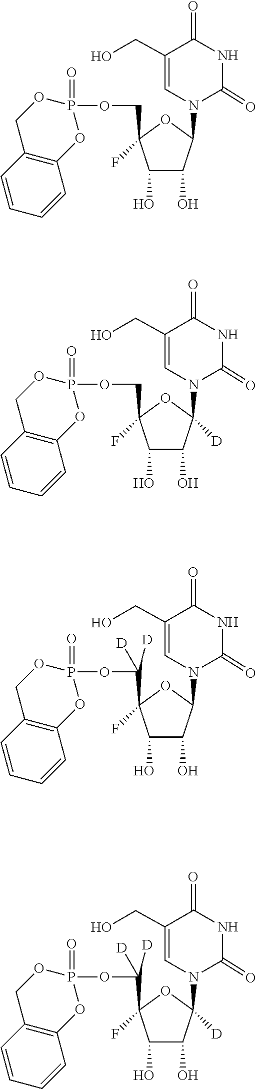 [SMILES: O=c1[nH]c(=O)n([C@@H]2O[C@](F)(COP3(=O)OCc4ccccc4O3)[C@@H](O)[C@H]2O)cc1CO ; [2H][C@@]1(n2cc(CO)c(=O)[nH]c2=O)O[C@](F)(COP2(=O)OCc3ccccc3O2)[C@@H](O)[C@H]1O ; [2H]C([2H])(OP1(=O)OCc2ccccc2O1)[C@@]1(F)O[C@@H](n2cc(CO)c(=O)[nH]c2=O)[C@H](O)[C@@H]1O ; [2H]C([2H])(OP1(=O)OCc2ccccc2O1)[C@@]1(F)O[C@@]([2H])(n2cc(CO)c(=O)[nH]c2=O)[C@H](O)[C@@H]1O]

In exemplary embodiments, the compound is selected from:

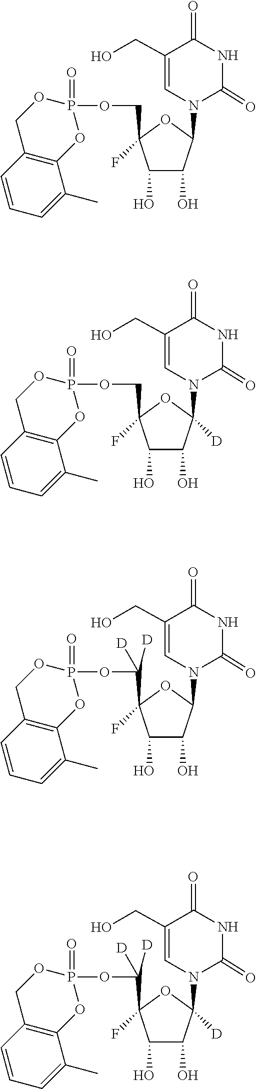 [SMILES: Cc1cccc2c1OP(=O)(OC[C@@]1(F)O[C@@H](n3cc(CO)c(=O)[nH]c3=O)[C@H](O)[C@@H]1O)OC2 ; [2H][C@@]1(n2cc(CO)c(=O)[nH]c2=O)O[C@](F)(COP2(=O)OCc3cccc(C)c3O2)[C@@H](O)[C@H]1O ; [2H]C([2H])(OP1(=O)OCc2cccc(C)c2O1)[C@@]1(F)O[C@@H](n2cc(CO)c(=O)[nH]c2=O)[C@H](O)[C@@H]1O ; [2H]C([2H])(OP1(=O)OCc2cccc(C)c2O1)[C@@]1(F)O[C@@]([2H])(n2cc(CO)c(=O)[nH]c2=O)[C@H](O)[C@@H]1O]

In exemplary embodiments, the compound is selected from:

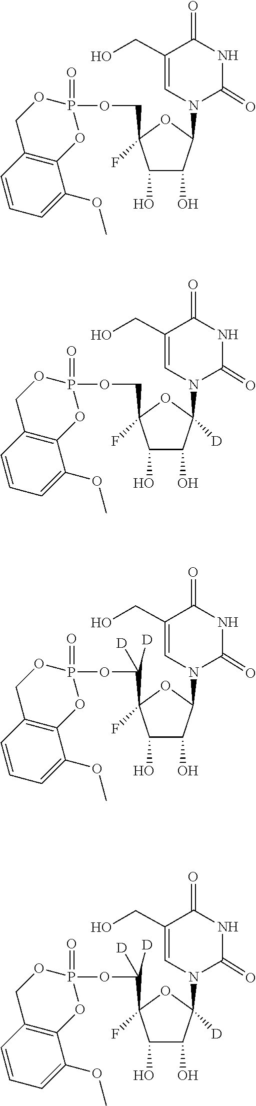 [SMILES: COc1cccc2c1OP(=O)(OC[C@@]1(F)O[C@@H](n3cc(CO)c(=O)[nH]c3=O)[C@H](O)[C@@H]1O)OC2 ; [2H][C@@]1(n2cc(CO)c(=O)[nH]c2=O)O[C@](F)(COP2(=O)OCc3cccc(OC)c3O2)[C@@H](O)[C@H]1O ; [2H]C([2H])(OP1(=O)OCc2cccc(OC)c2O1)[C@@]1(F)O[C@@H](n2cc(CO)c(=O)[nH]c2=O)[C@H](O)[C@@H]1O ; [2H]C([2H])(OP1(=O)OCc2cccc(OC)c2O1)[C@@]1(F)O[C@@]([2H])(n2cc(CO)c(=O)[nH]c2=O)[C@H](O)[C@@H]1O]

In exemplary embodiments, the compound is selected from:

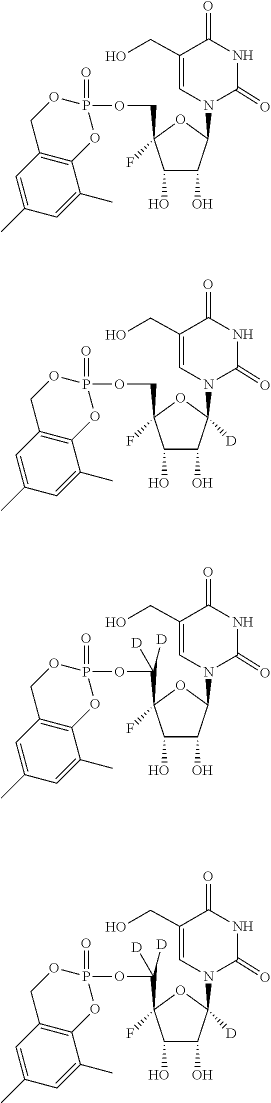 [SMILES: Cc1cc(C)c2c(c1)COP(=O)(OC[C@@]1(F)O[C@@H](n3cc(CO)c(=O)[nH]c3=O)[C@H](O)[C@@H]1O)O2 ; [2H][C@@]1(n2cc(CO)c(=O)[nH]c2=O)O[C@](F)(COP2(=O)OCc3cc(C)cc(C)c3O2)[C@@H](O)[C@H]1O ; [2H]C([2H])(OP1(=O)OCc2cc(C)cc(C)c2O1)[C@@]1(F)O[C@@H](n2cc(CO)c(=O)[nH]c2=O)[C@H](O)[C@@H]1O ; [2H]C([2H])(OP1(=O)OCc2cc(C)cc(C)c2O1)[C@@]1(F)O[C@@]([2H])(n2cc(CO)c(=O)[nH]c2=O)[C@H](O)[C@@H]1O]

In exemplary embodiments, the compound is selected from:

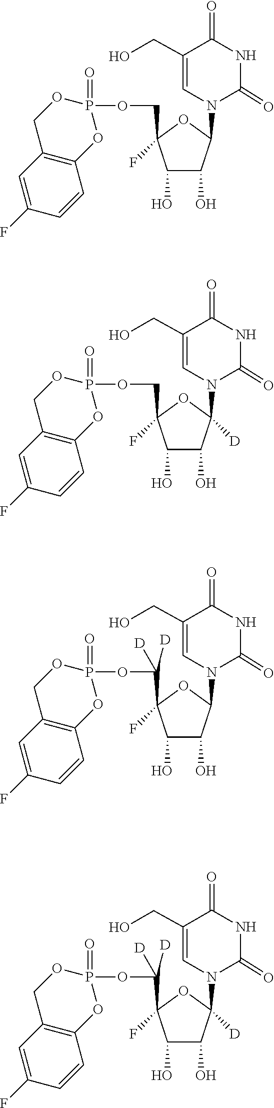 [SMILES: O=c1[nH]c(=O)n([C@@H]2O[C@](F)(COP3(=O)OCc4cc(F)ccc4O3)[C@@H](O)[C@H]2O)cc1CO ; [2H][C@@]1(n2cc(CO)c(=O)[nH]c2=O)O[C@](F)(COP2(=O)OCc3cc(F)ccc3O2)[C@@H](O)[C@H]1O ; [2H]C([2H])(OP1(=O)OCc2cc(F)ccc2O1)[C@@]1(F)O[C@@H](n2cc(CO)c(=O)[nH]c2=O)[C@H](O)[C@@H]1O ; [2H]C([2H])(OP1(=O)OCc2cc(F)ccc2O1)[C@@]1(F)O[C@@]([2H])(n2cc(CO)c(=O)[nH]c2=O)[C@H](O)[C@@H]1O]

In exemplary embodiments, the compound is selected from:

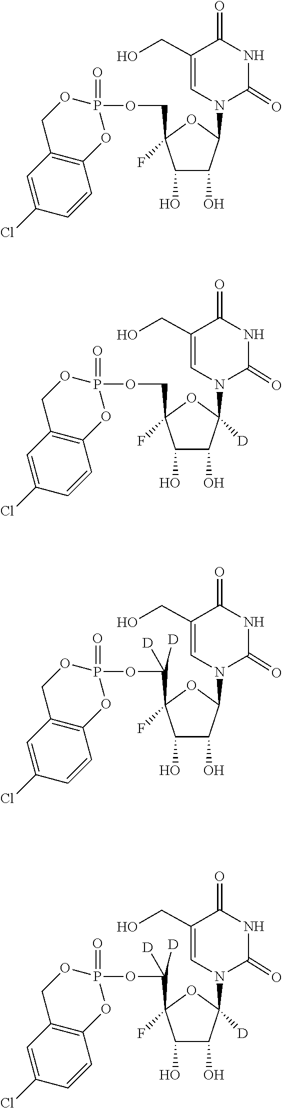 [SMILES: O=c1[nH]c(=O)n([C@@H]2O[C@](F)(COP3(=O)OCc4cc(Cl)ccc4O3)[C@@H](O)[C@H]2O)cc1CO ; [2H][C@@]1(n2cc(CO)c(=O)[nH]c2=O)O[C@](F)(COP2(=O)OCc3cc(Cl)ccc3O2)[C@@H](O)[C@H]1O ; [2H]C([2H])(OP1(=O)OCc2cc(Cl)ccc2O1)[C@@]1(F)O[C@@H](n2cc(CO)c(=O)[nH]c2=O)[C@H](O)[C@@H]1O ; [2H]C([2H])(OP1(=O)OCc2cc(Cl)ccc2O1)[C@@]1(F)O[C@@]([2H])(n2cc(CO)c(=O)[nH]c2=O)[C@H](O)[C@@H]1O]

In exemplary embodiments, the compound is selected from:

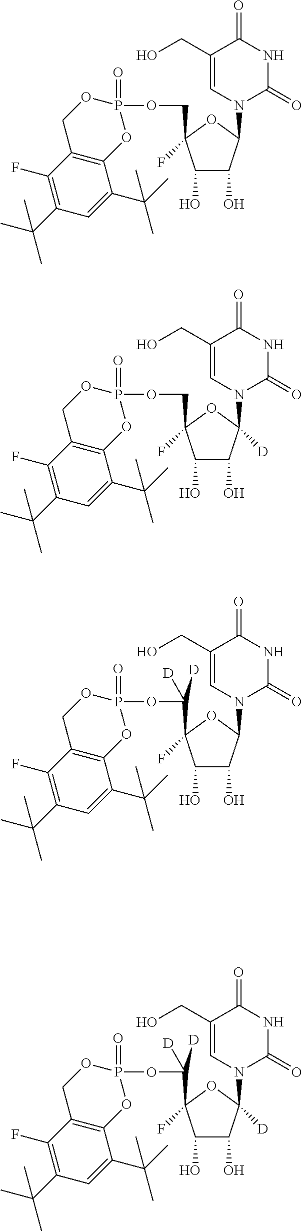

In exemplary embodiments, the compound is selected from:

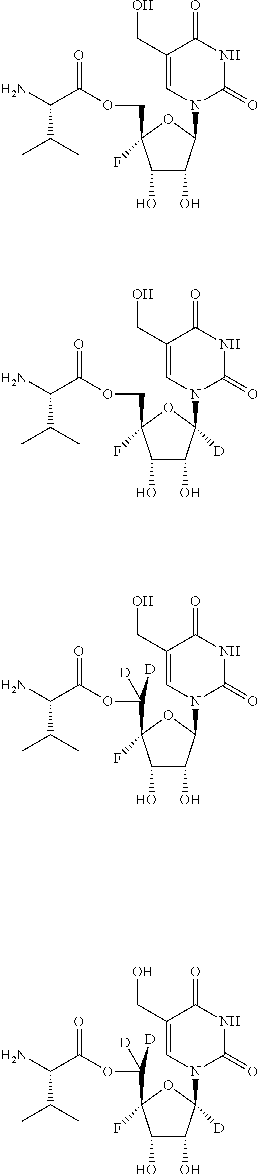

In exemplary embodiments, the compound is selected from:

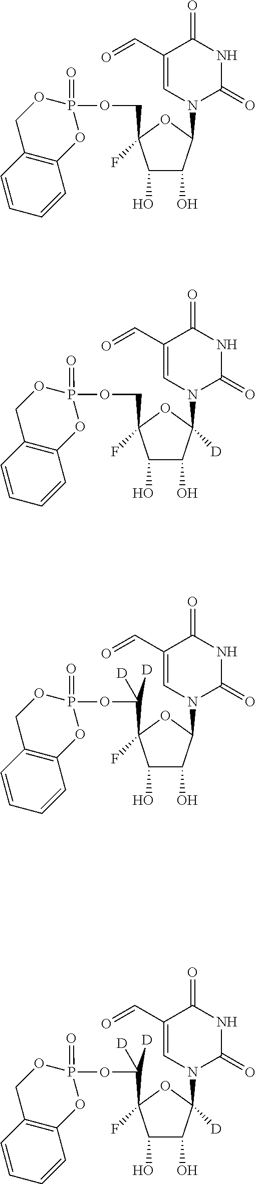

In exemplary embodiments, the compound is selected from:

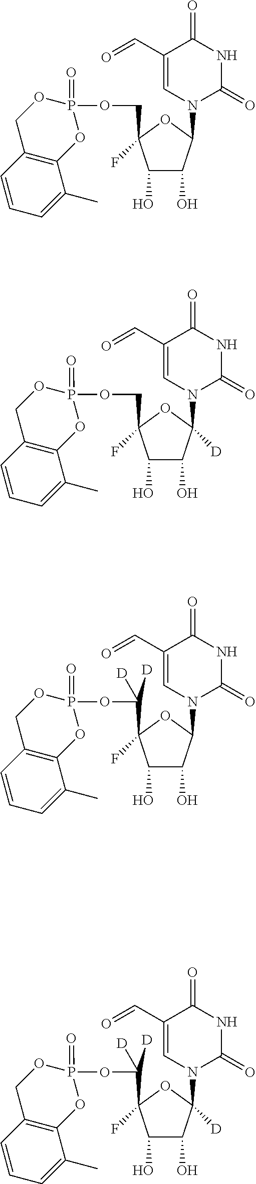

In exemplary embodiments, the compound is selected from:

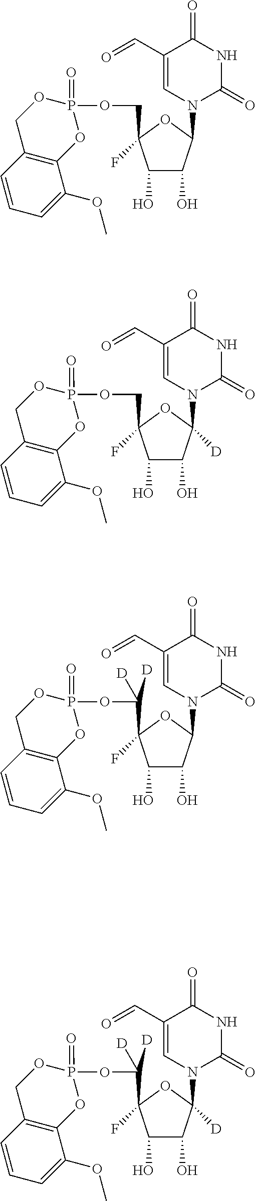

In exemplary embodiments, the compound is selected from:

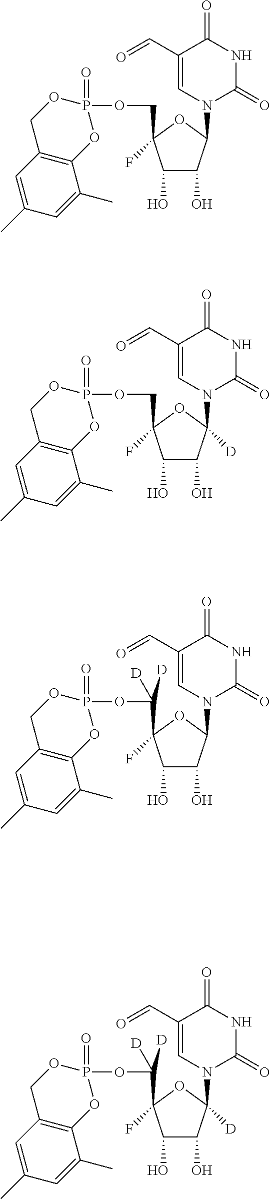

In exemplary embodiments, the compound is selected from:

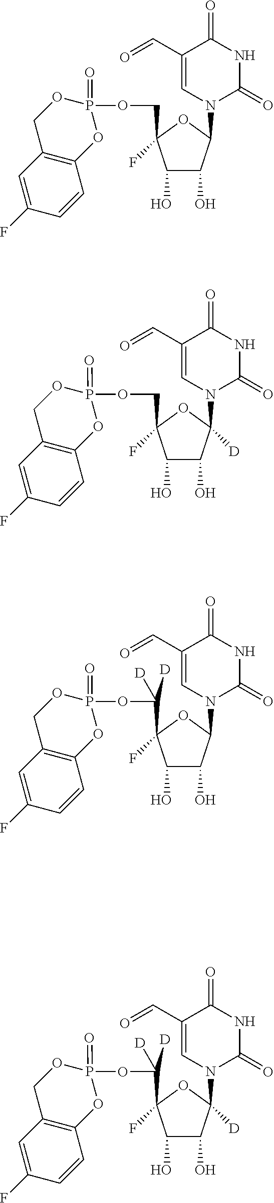

In exemplary embodiments, the compound is selected from:

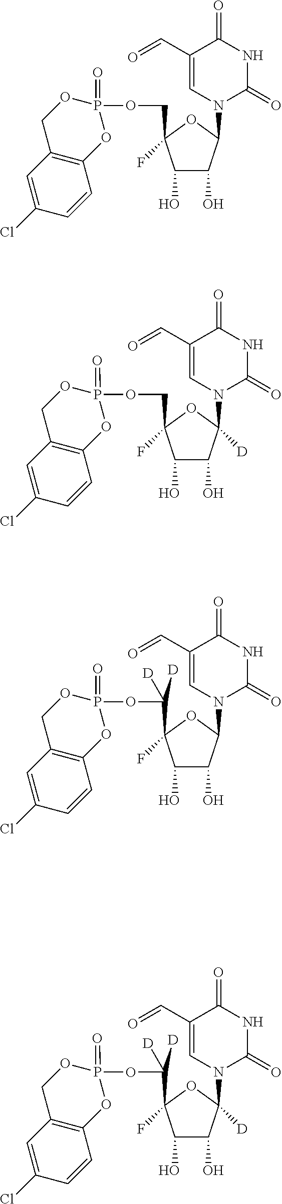

In exemplary embodiments, the compound is selected from:

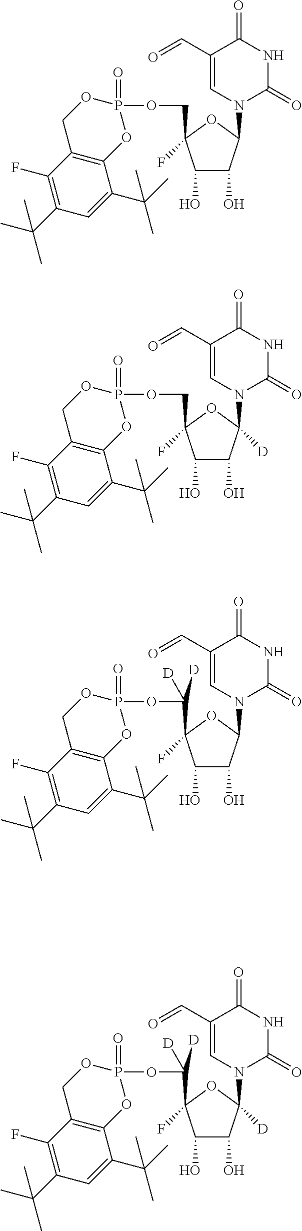

In exemplar embodiments, the compound is selected from:

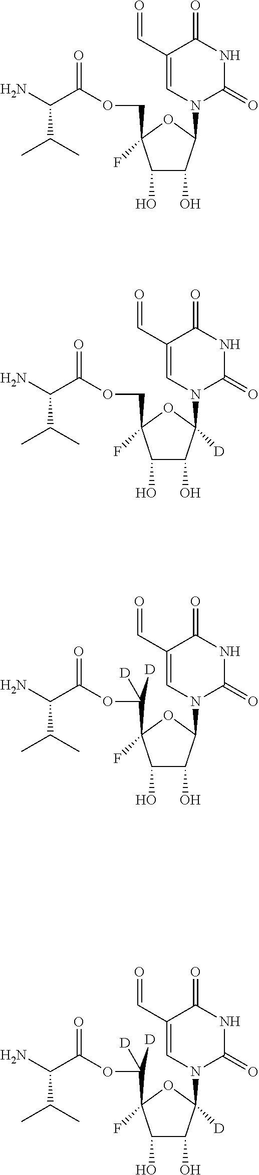

In exemplary embodiments, the compound is selected from:

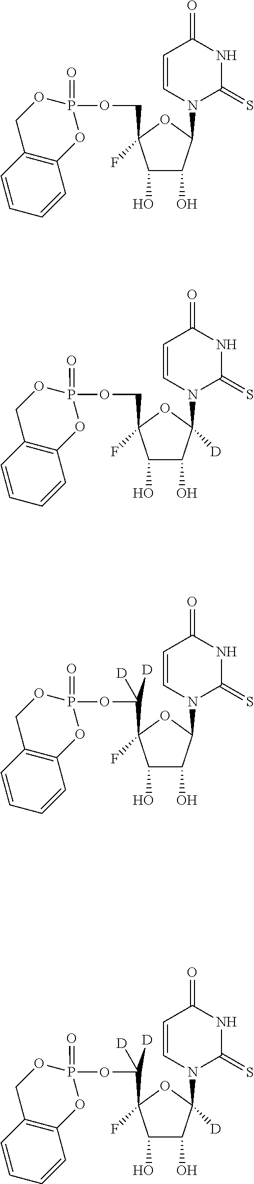

In exemplary embodiments, the compound is selected from:

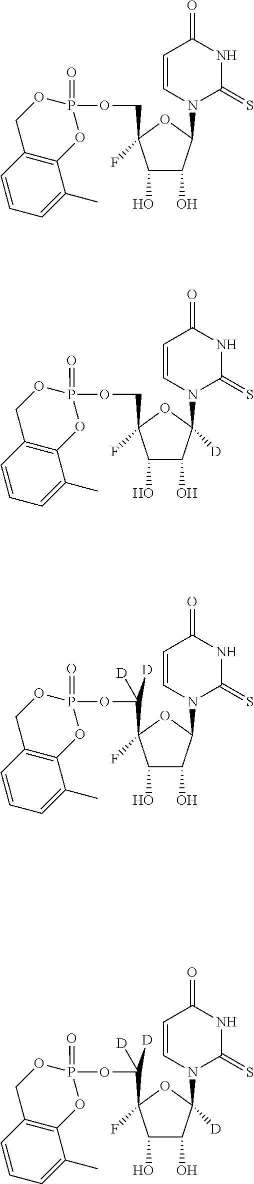

In exemplary embodiments, the compound is selected from:

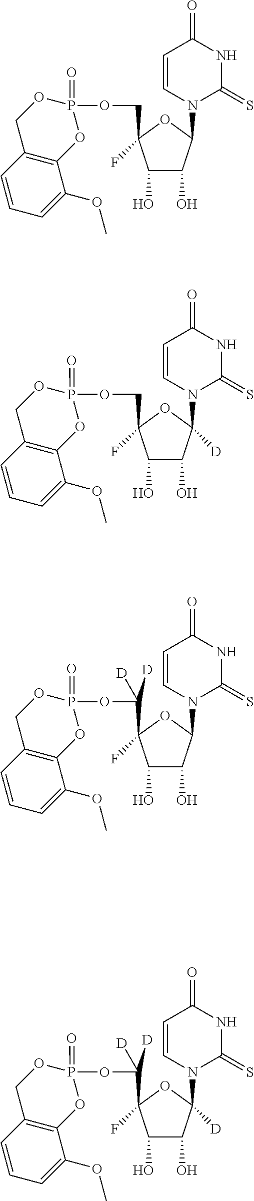

In exemplary embodiments, the compound is selected from:

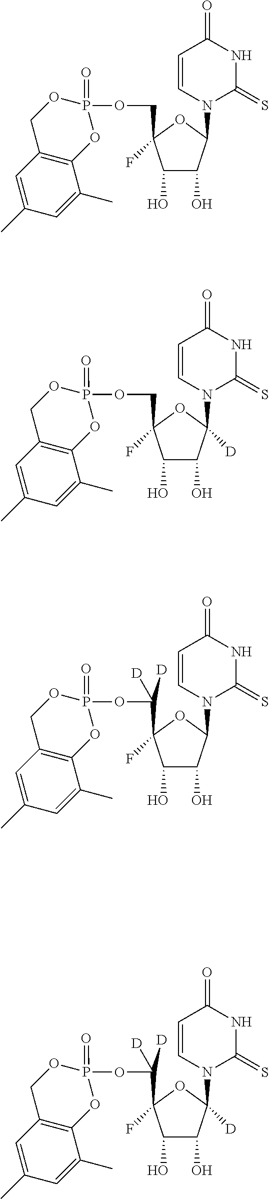

In exemplary embodiments, the compound is selected from:

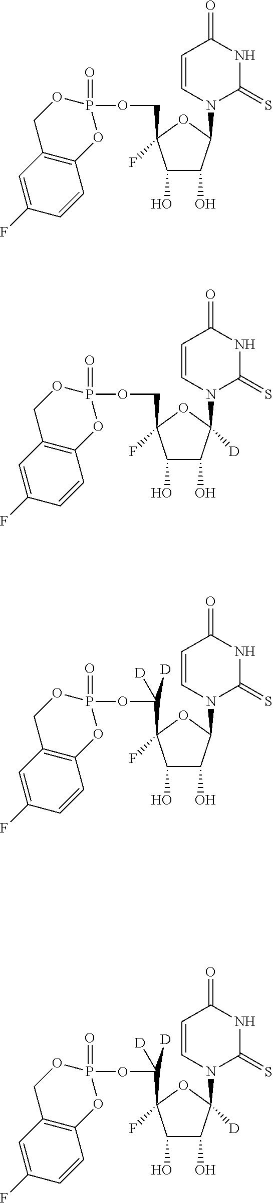

In exemplary embodiments, the compound is selected from:

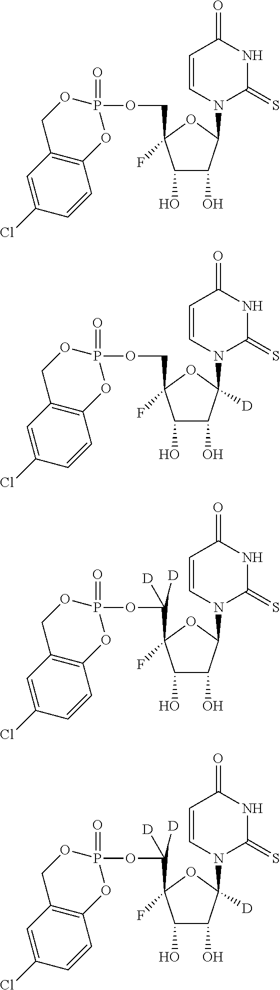 [SMILES: O=c1ccn([C@@H]2O[C@](F)(COP3(=O)OCc4cc(Cl)ccc4O3)[C@@H](O)[C@H]2O)c(=S)[nH]1 ; [2H][C@@]1(n2ccc(=O)[nH]c2=S)O[C@](F)(COP2(=O)OCc3cc(Cl)ccc3O2)[C@@H](O)[C@H]1O ; [2H]C([2H])(OP1(=O)OCc2cc(Cl)ccc2O1)[C@@]1(F)O[C@@H](n2ccc(=O)[nH]c2=S)[C@H](O)[C@@H]1O ; [2H]C([2H])(OP1(=O)OCc2cc(Cl)ccc2O1)[C@@]1(F)O[C@@]([2H])(n2ccc(=O)[nH]c2=S)[C@H](O)[C@@H]1O]

In exemplary embodiments, the compound is selected from:

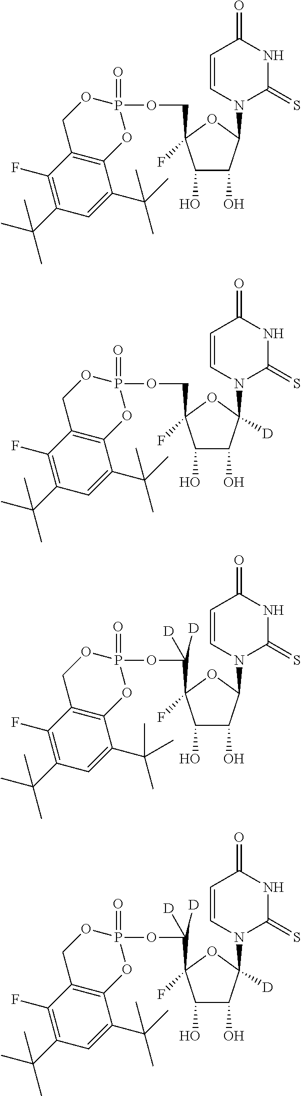 [SMILES: CC(C)(C)c1cc(C(C)(C)C)c2c(c1F)COP(=O)(OC[C@@]1(F)O[C@@H](n3ccc(=O)[nH]c3=S)[C@H](O)[C@@H]1O)O2 ; [2H][C@@]1(n2ccc(=O)[nH]c2=S)O[C@](F)(COP2(=O)OCc3c(F)c(C(C)(C)C)cc(C(C)(C)C)c3O2)[C@@H](O)[C@H]1O ; [2H]C([2H])(OP1(=O)OCc2c(F)c(C(C)(C)C)cc(C(C)(C)C)c2O1)[C@@]1(F)O[C@@H](n2ccc(=O)[nH]c2=S)[C@H](O)[C@@H]1O ; [2H]C([2H])(OP1(=O)OCc2c(F)c(C(C)(C)C)cc(C(C)(C)C)c2O1)[C@@]1(F)O[C@@]([2H])(n2ccc(=O)[nH]c2=S)[C@H](O)[C@@H]1O]

In exemplary embodiments, the compound is selected from:

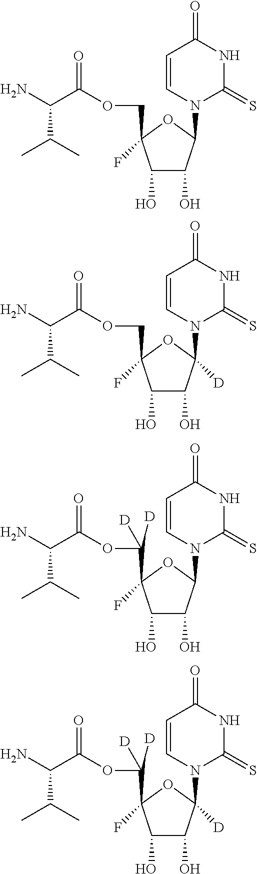

In exemplary embodiments, the compound is selected from:

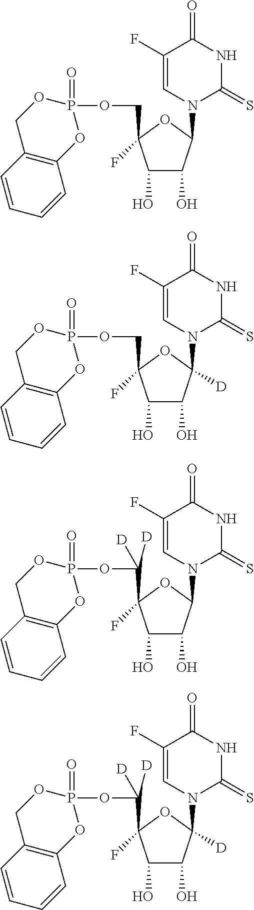

In exemplary embodiments, the compound is selected from:

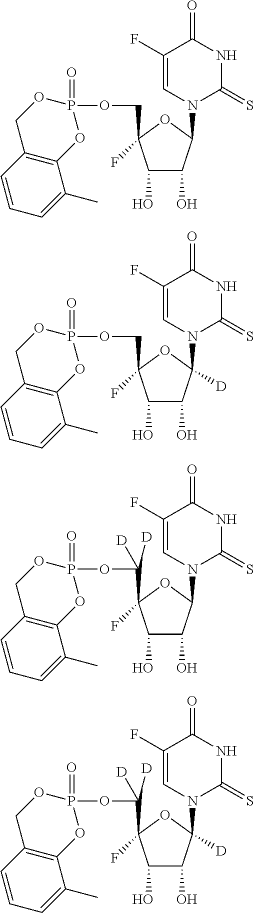

In exemplary embodiments, the compound is selected from:

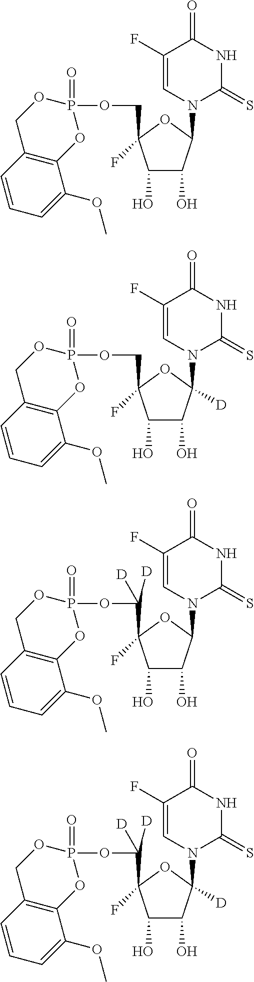 [SMILES: COc1cccc2c1OP(=O)(OC[C@@]1(F)O[C@@H](n3cc(F)c(=O)[nH]c3=S)[C@H](O)[C@@H]1O)OC2 ; [2H][C@@]1(n2cc(F)c(=O)[nH]c2=S)O[C@](F)(COP2(=O)OCc3cccc(OC)c3O2)[C@@H](O)[C@H]1O ; [2H]C([2H])(OP1(=O)OCc2cccc(OC)c2O1)[C@@]1(F)O[C@@H](n2cc(F)c(=O)[nH]c2=S)[C@H](O)[C@@H]1O ; [2H]C([2H])(OP1(=O)OCc2cccc(OC)c2O1)[C@@]1(F)O[C@@]([2H])(n2cc(F)c(=O)[nH]c2=S)[C@H](O)[C@@H]1O]

In exemplary embodiments, the compound is selected from:

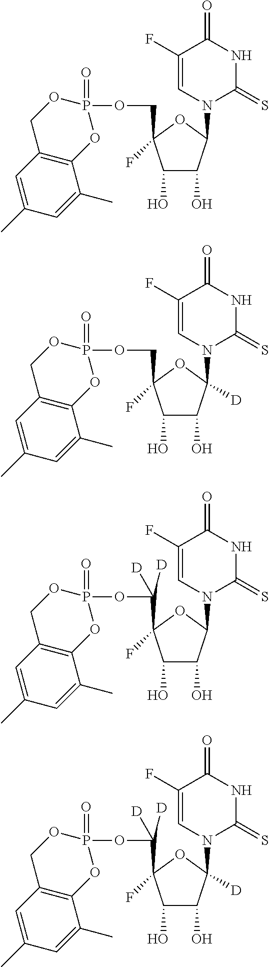

In exemplary embodiments, the compound is selected from:

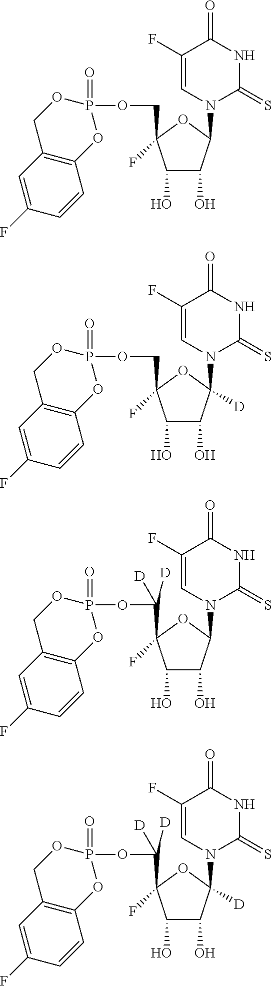 [SMILES: O=c1[nH]c(=S)n([C@@H]2O[C@](F)(COP3(=O)OCc4cc(F)ccc4O3)[C@@H](O)[C@H]2O)cc1F ; [2H][C@@]1(n2cc(F)c(=O)[nH]c2=S)O[C@](F)(COP2(=O)OCc3cc(F)ccc3O2)[C@@H](O)[C@H]1O ; [2H]C([2H])(OP1(=O)OCc2cc(F)ccc2O1)[C@@]1(F)O[C@@H](n2cc(F)c(=O)[nH]c2=S)[C@H](O)[C@@H]1O ; [2H]C([2H])(OP1(=O)OCc2cc(F)ccc2O1)[C@@]1(F)O[C@@]([2H])(n2cc(F)c(=O)[nH]c2=S)[C@H](O)[C@@H]1O]

In exemplary embodiments, the compound is selected from:

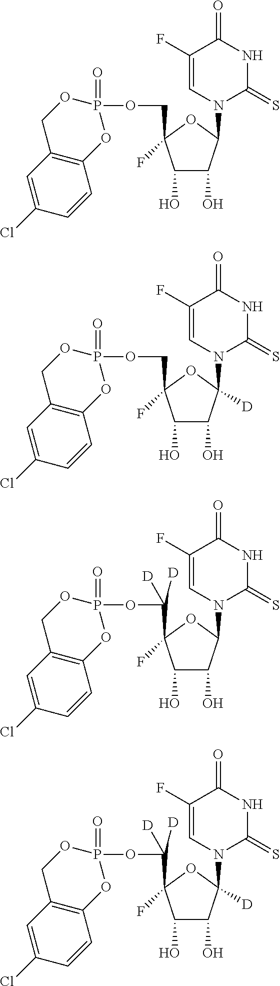 [SMILES: O=c1[nH]c(=S)n([C@@H]2O[C@](F)(COP3(=O)OCc4cc(Cl)ccc4O3)[C@@H](O)[C@H]2O)cc1F ; [2H][C@@]1(n2cc(F)c(=O)[nH]c2=S)O[C@](F)(COP2(=O)OCc3cc(Cl)ccc3O2)[C@@H](O)[C@H]1O ; [2H]C([2H])(OP1(=O)OCc2cc(Cl)ccc2O1)[C@@]1(F)O[C@@H](n2cc(F)c(=O)[nH]c2=S)[C@H](O)[C@@H]1O ; [2H]C([2H])(OP1(=O)OCc2cc(Cl)ccc2O1)[C@@]1(F)O[C@@]([2H])(n2cc(F)c(=O)[nH]c2=S)[C@H](O)[C@@H]1O]

In exemplary embodiments, the compound is selected from:

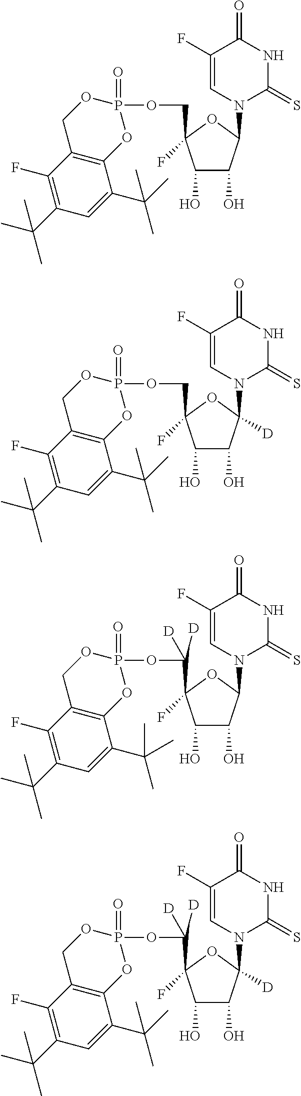 [SMILES: CC(C)(C)c1cc(C(C)(C)C)c2c(c1F)COP(=O)(OC[C@@]1(F)O[C@@H](n3cc(F)c(=O)[nH]c3=S)[C@H](O)[C@@H]1O)O2 ; [2H][C@@]1(n2cc(F)c(=O)[nH]c2=S)O[C@](F)(COP2(=O)OCc3c(F)c(C(C)(C)C)cc(C(C)(C)C)c3O2)[C@@H](O)[C@H]1O ; [2H]C([2H])(OP1(=O)OCc2c(F)c(C(C)(C)C)cc(C(C)(C)C)c2O1)[C@@]1(F)O[C@@H](n2cc(F)c(=O)[nH]c2=S)[C@H](O)[C@@H]1O ; [2H]C([2H])(OP1(=O)OCc2c(F)c(C(C)(C)C)cc(C(C)(C)C)c2O1)[C@@]1(F)O[C@@]([2H])(n2cc(F)c(=O)[nH]c2=S)[C@H](O)[C@@H]1O]

In exemplary embodiments, the compound is selected from:

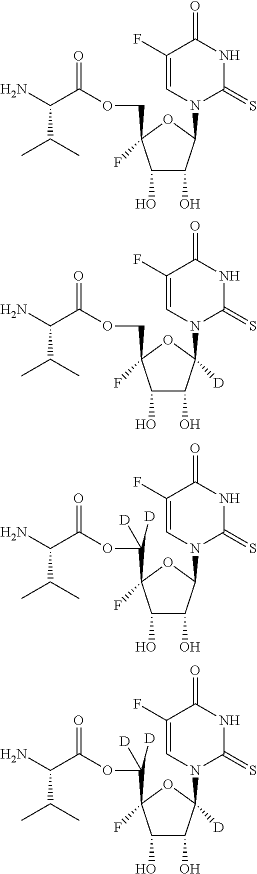

In exemplary embodiments, the compound is selected from:

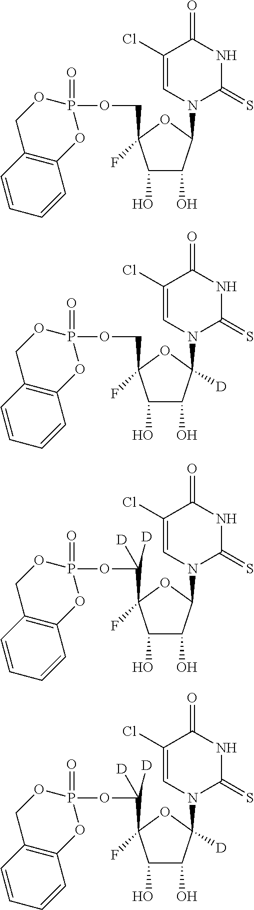

In exemplary embodiments, the compound is selected from:

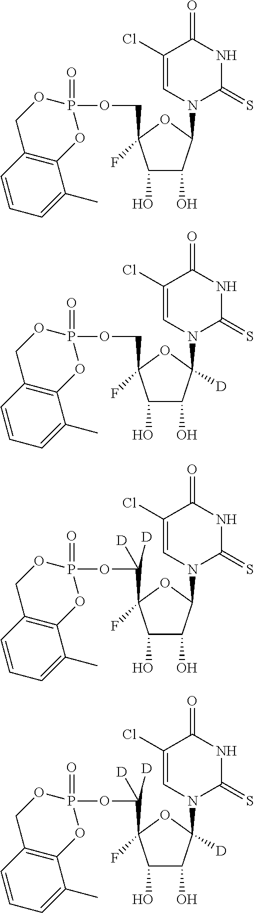

In exemplary embodiments, the compound is selected from:

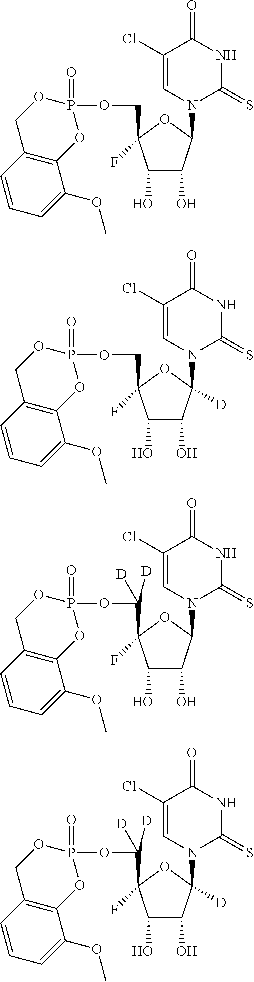 [SMILES: COc1cccc2c1OP(=O)(OC[C@@]1(F)O[C@@H](n3cc(Cl)c(=O)[nH]c3=S)[C@H](O)[C@@H]1O)OC2 ; [2H][C@@]1(n2cc(Cl)c(=O)[nH]c2=S)O[C@](F)(COP2(=O)OCc3cccc(OC)c3O2)[C@@H](O)[C@H]1O ; [2H]C([2H])(OP1(=O)OCc2cccc(OC)c2O1)[C@@]1(F)O[C@@H](n2cc(Cl)c(=O)[nH]c2=S)[C@H](O)[C@@H]1O ; [2H]C([2H])(OP1(=O)OCc2cccc(OC)c2O1)[C@@]1(F)O[C@@]([2H])(n2cc(Cl)c(=O)[nH]c2=S)[C@H](O)[C@@H]1O]

In exemplary embodiments, the compound is selected from:

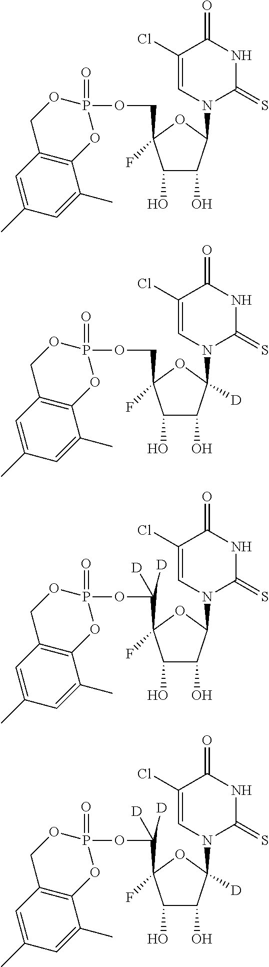

In exemplary embodiments, the compound is selected from:

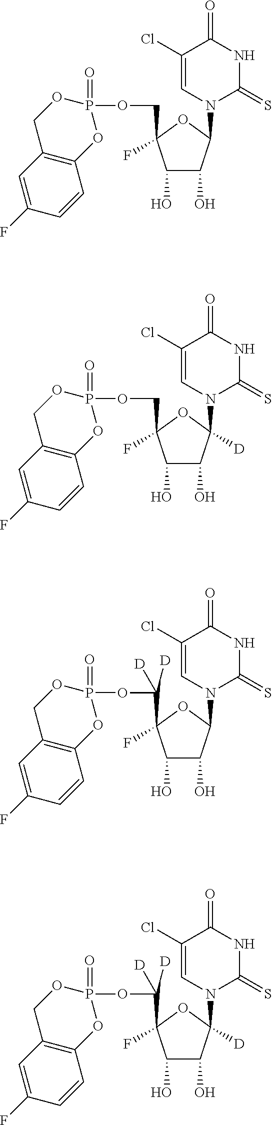 [SMILES: O=c1[nH]c(=S)n([C@@H]2O[C@](F)(COP3(=O)OCc4cc(F)ccc4O3)[C@@H](O)[C@H]2O)cc1Cl ; [2H][C@@]1(n2cc(Cl)c(=O)[nH]c2=S)O[C@](F)(COP2(=O)OCc3cc(F)ccc3O2)[C@@H](O)[C@H]1O ; [2H]C([2H])(OP1(=O)OCc2cc(F)ccc2O1)[C@@]1(F)O[C@@H](n2cc(Cl)c(=O)[nH]c2=S)[C@H](O)[C@@H]1O ; [2H]C([2H])(OP1(=O)OCc2cc(F)ccc2O1)[C@@]1(F)O[C@@]([2H])(n2cc(Cl)c(=O)[nH]c2=S)[C@H](O)[C@@H]1O]

In exemplary embodiments, the compound is selected from:

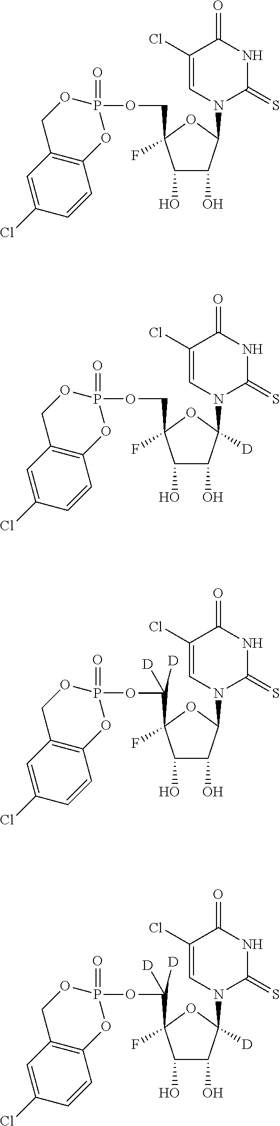 [SMILES: O=c1[nH]c(=S)n([C@@H]2O[C@](F)(COP3(=O)OCc4cc(Cl)ccc4O3)[C@@H](O)[C@H]2O)cc1Cl ; [2H][C@@]1(n2cc(Cl)c(=O)[nH]c2=S)O[C@](F)(COP2(=O)OCc3cc(Cl)ccc3O2)[C@@H](O)[C@H]1O ; [2H]C([2H])(OP1(=O)OCc2cc(Cl)ccc2O1)[C@@]1(F)O[C@@H](n2cc(Cl)c(=O)[nH]c2=S)[C@H](O)[C@@H]1O ; [2H]C([2H])(OP1(=O)OCc2cc(Cl)ccc2O1)[C@@]1(F)O[C@@]([2H])(n2cc(Cl)c(=O)[nH]c2=S)[C@H](O)[C@@H]1O]

In exemplary embodiments, the compound is selected from:

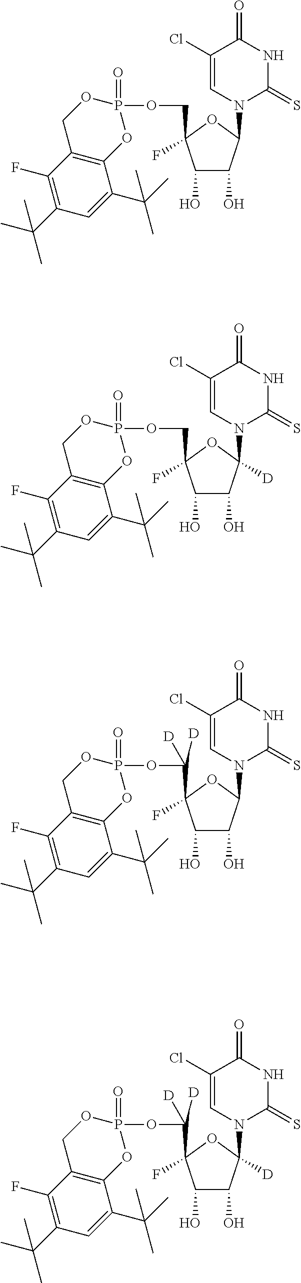 [SMILES: CC(C)(C)c1cc(C(C)(C)C)c2c(c1F)COP(=O)(OC[C@@]1(F)O[C@@H](n3cc(Cl)c(=O)[nH]c3=S)[C@H](O)[C@@H]1O)O2 ; [2H][C@@]1(n2cc(Cl)c(=O)[nH]c2=S)O[C@](F)(COP2(=O)OCc3c(F)c(C(C)(C)C)cc(C(C)(C)C)c3O2)[C@@H](O)[C@H]1O ; [2H]C([2H])(OP1(=O)OCc2c(F)c(C(C)(C)C)cc(C(C)(C)C)c2O1)[C@@]1(F)O[C@@H](n2cc(Cl)c(=O)[nH]c2=S)[C@H](O)[C@@H]1O ; [2H]C([2H])(OP1(=O)OCc2c(F)c(C(C)(C)C)cc(C(C)(C)C)c2O1)[C@@]1(F)O[C@@]([2H])(n2cc(Cl)c(=O)[nH]c2=S)[C@H](O)[C@@H]1O]

In exemplary embodiments, the compound is selected from:

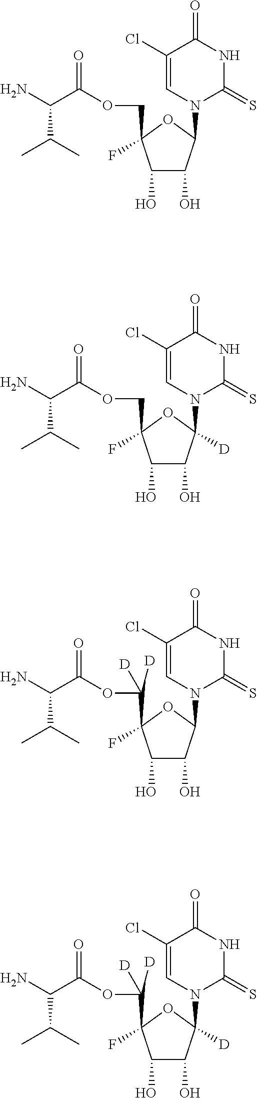

In exemplary embodiments, the compound is selected from:

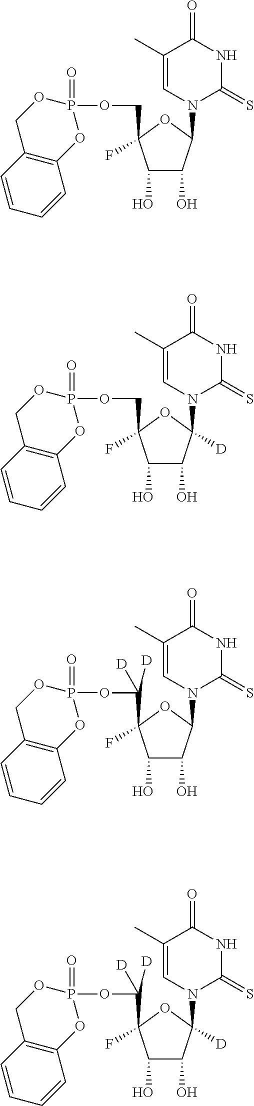 [SMILES: Cc1cn([C@@H]2O[C@](F)(COP3(=O)OCc4ccccc4O3)[C@@H](O)[C@H]2O)c(=S)[nH]c1=O ; [2H][C@@]1(n2cc(C)c(=O)[nH]c2=S)O[C@](F)(COP2(=O)OCc3ccccc3O2)[C@@H](O)[C@H]1O ; [2H]C([2H])(OP1(=O)OCc2ccccc2O1)[C@@]1(F)O[C@@H](n2cc(C)c(=O)[nH]c2=S)[C@H](O)[C@@H]1O ; [2H]C([2H])(OP1(=O)OCc2ccccc2O1)[C@@]1(F)O[C@@]([2H])(n2cc(C)c(=O)[nH]c2=S)[C@H](O)[C@@H]1O]

In exemplary embodiments, the compound is selected from:

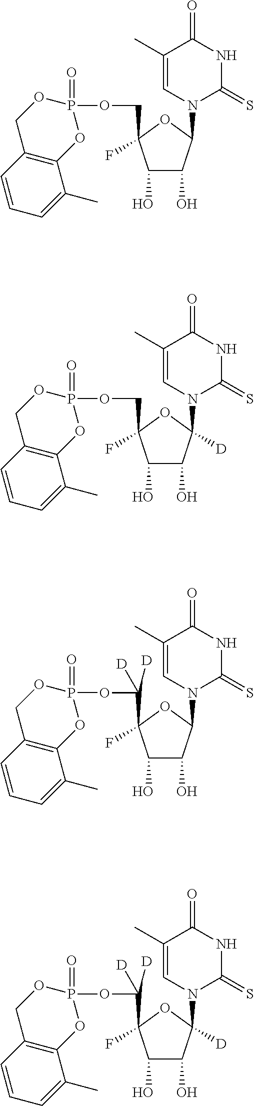 [SMILES: Cc1cccc2c1OP(=O)(OC[C@@]1(F)O[C@@H](n3cc(C)c(=O)[nH]c3=S)[C@H](O)[C@@H]1O)OC2 ; [2H][C@@]1(n2cc(C)c(=O)[nH]c2=S)O[C@](F)(COP2(=O)OCc3cccc(C)c3O2)[C@@H](O)[C@H]1O ; [2H]C([2H])(OP1(=O)OCc2cccc(C)c2O1)[C@@]1(F)O[C@@H](n2cc(C)c(=O)[nH]c2=S)[C@H](O)[C@@H]1O ; [2H]C([2H])(OP1(=O)OCc2cccc(C)c2O1)[C@@]1(F)O[C@@]([2H])(n2cc(C)c(=O)[nH]c2=S)[C@H](O)[C@@H]1O]

In exemplary embodiments, the compound is selected from:

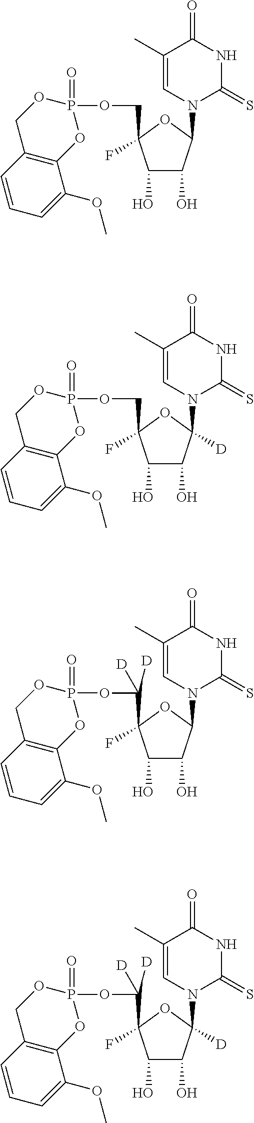 [SMILES: COc1cccc2c1OP(=O)(OC[C@@]1(F)O[C@@H](n3cc(C)c(=O)[nH]c3=S)[C@H](O)[C@@H]1O)OC2 ; [2H][C@@]1(n2cc(C)c(=O)[nH]c2=S)O[C@](F)(COP2(=O)OCc3cccc(OC)c3O2)[C@@H](O)[C@H]1O ; [2H]C([2H])(OP1(=O)OCc2cccc(OC)c2O1)[C@@]1(F)O[C@@H](n2cc(C)c(=O)[nH]c2=S)[C@H](O)[C@@H]1O ; [2H]C([2H])(OP1(=O)OCc2cccc(OC)c2O1)[C@@]1(F)O[C@@]([2H])(n2cc(C)c(=O)[nH]c2=S)[C@H](O)[C@@H]1O]

In exemplary embodiments, the compound is selected from:

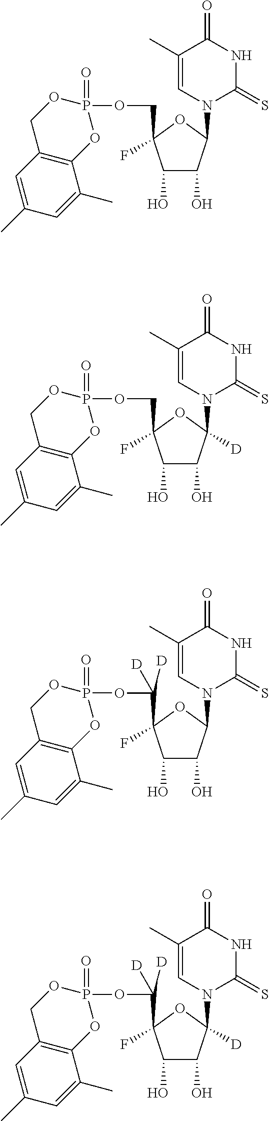 [SMILES: Cc1cc(C)c2c(c1)COP(=O)(OC[C@@]1(F)O[C@@H](n3cc(C)c(=O)[nH]c3=S)[C@H](O)[C@@H]1O)O2 ; [2H][C@@]1(n2cc(C)c(=O)[nH]c2=S)O[C@](F)(COP2(=O)OCc3cc(C)cc(C)c3O2)[C@@H](O)[C@H]1O ; [2H]C([2H])(OP1(=O)OCc2cc(C)cc(C)c2O1)[C@@]1(F)O[C@@H](n2cc(C)c(=O)[nH]c2=S)[C@H](O)[C@@H]1O ; [2H]C([2H])(OP1(=O)OCc2cc(C)cc(C)c2O1)[C@@]1(F)O[C@@]([2H])(n2cc(C)c(=O)[nH]c2=S)[C@H](O)[C@@H]1O]

In exemplary embodiments, the compound is selected from:

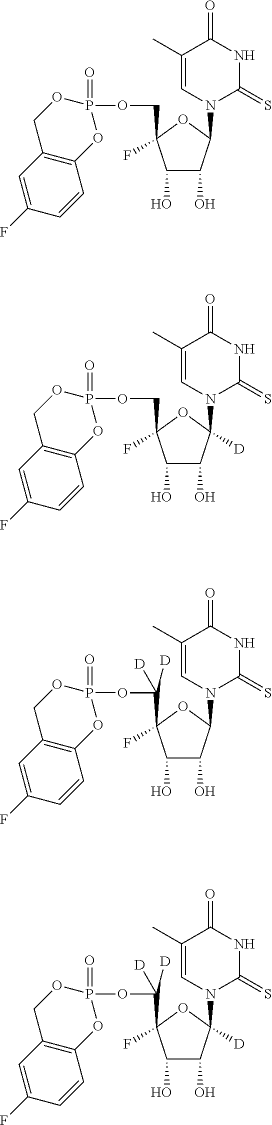 [SMILES: Cc1cn([C@@H]2O[C@](F)(COP3(=O)OCc4cc(F)ccc4O3)[C@@H](O)[C@H]2O)c(=S)[nH]c1=O ; [2H][C@@]1(n2cc(C)c(=O)[nH]c2=S)O[C@](F)(COP2(=O)OCc3cc(F)ccc3O2)[C@@H](O)[C@H]1O ; [2H]C([2H])(OP1(=O)OCc2cc(F)ccc2O1)[C@@]1(F)O[C@@H](n2cc(C)c(=O)[nH]c2=S)[C@H](O)[C@@H]1O ; [2H]C([2H])(OP1(=O)OCc2cc(F)ccc2O1)[C@@]1(F)O[C@@]([2H])(n2cc(C)c(=O)[nH]c2=S)[C@H](O)[C@@H]1O]

In exemplary embodiments, the compound is selected from:

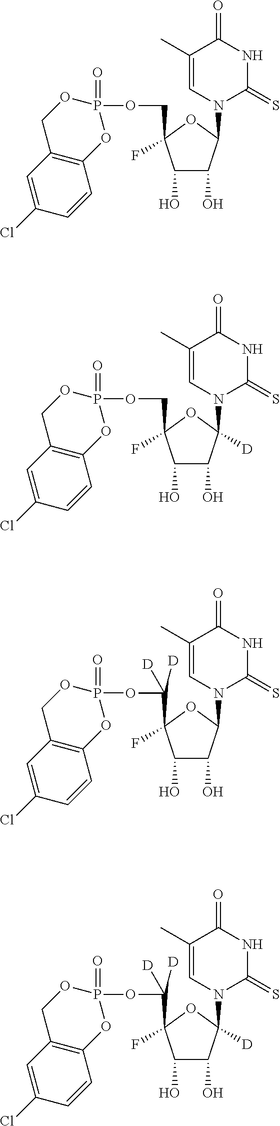 [SMILES: Cc1cn([C@@H]2O[C@](F)(COP3(=O)OCc4cc(Cl)ccc4O3)[C@@H](O)[C@H]2O)c(=S)[nH]c1=O ; [2H][C@@]1(n2cc(C)c(=O)[nH]c2=S)O[C@](F)(COP2(=O)OCc3cc(Cl)ccc3O2)[C@@H](O)[C@H]1O ; [2H]C([2H])(OP1(=O)OCc2cc(Cl)ccc2O1)[C@@]1(F)O[C@@H](n2cc(C)c(=O)[nH]c2=S)[C@H](O)[C@@H]1O ; [2H]C([2H])(OP1(=O)OCc2cc(Cl)ccc2O1)[C@@]1(F)O[C@@]([2H])(n2cc(C)c(=O)[nH]c2=S)[C@H](O)[C@@H]1O]

In exemplary embodiments, the compound is selected from:

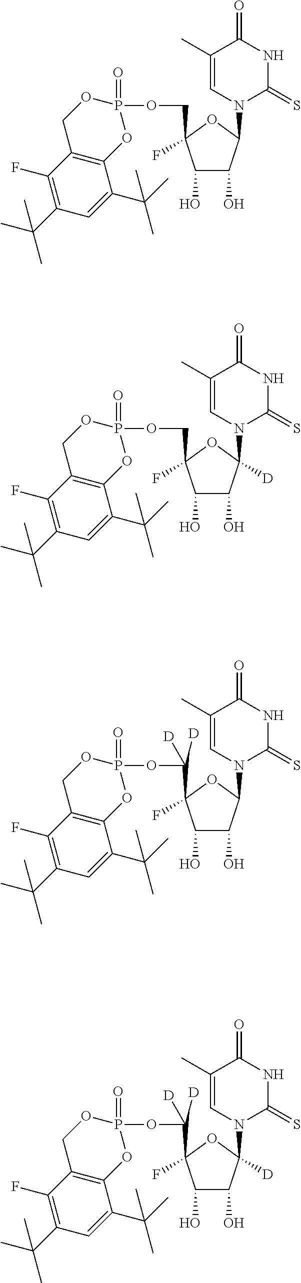 [SMILES: Cc1cn([C@@H]2O[C@](F)(COP3(=O)OCc4c(F)c(C(C)(C)C)cc(C(C)(C)C)c4O3)[C@@H](O)[C@H]2O)c(=S)[nH]c1=O ; [2H][C@@]1(n2cc(C)c(=O)[nH]c2=S)O[C@](F)(COP2(=O)OCc3c(F)c(C(C)(C)C)cc(C(C)(C)C)c3O2)[C@@H](O)[C@H]1O ; [2H]C([2H])(OP1(=O)OCc2c(F)c(C(C)(C)C)cc(C(C)(C)C)c2O1)[C@@]1(F)O[C@@H](n2cc(C)c(=O)[nH]c2=S)[C@H](O)[C@@H]1O ; [2H]C([2H])(OP1(=O)OCc2c(F)c(C(C)(C)C)cc(C(C)(C)C)c2O1)[C@@]1(F)O[C@@]([2H])(n2cc(C)c(=O)[nH]c2=S)[C@H](O)[C@@H]1O]

In exemplary embodiments, the compound is selected from:

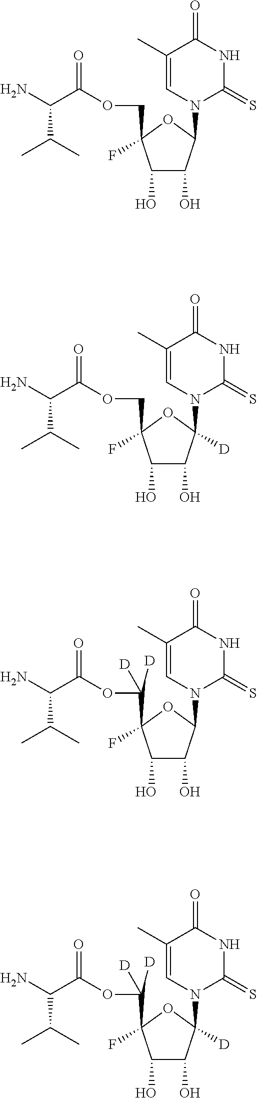

In exemplary embodiments, the compound is selected from:

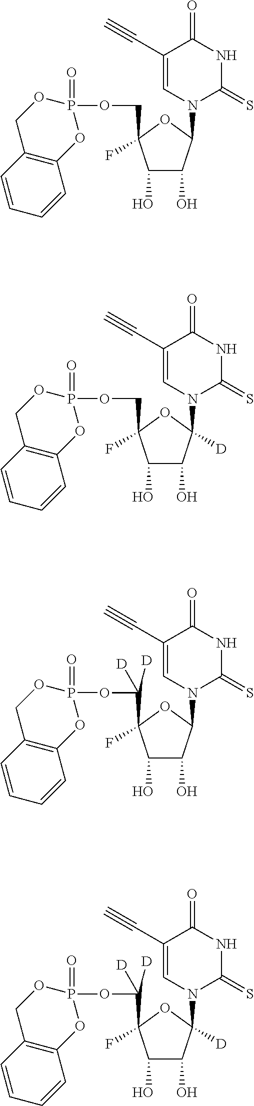 [SMILES: C#Cc1cn([C@@H]2O[C@](F)(COP3(=O)OCc4ccccc4O3)[C@@H](O)[C@H]2O)c(=S)[nH]c1=O ; [2H][C@@]1(n2cc(C#C)c(=O)[nH]c2=S)O[C@](F)(COP2(=O)OCc3ccccc3O2)[C@@H](O)[C@H]1O ; [2H]C([2H])(OP1(=O)OCc2ccccc2O1)[C@@]1(F)O[C@@H](n2cc(C#C)c(=O)[nH]c2=S)[C@H](O)[C@@H]1O ; [2H]C([2H])(OP1(=O)OCc2ccccc2O1)[C@@]1(F)O[C@@]([2H])(n2cc(C#C)c(=O)[nH]c2=S)[C@H](O)[C@@H]1O]

In exemplary embodiments, the compound is selected from:

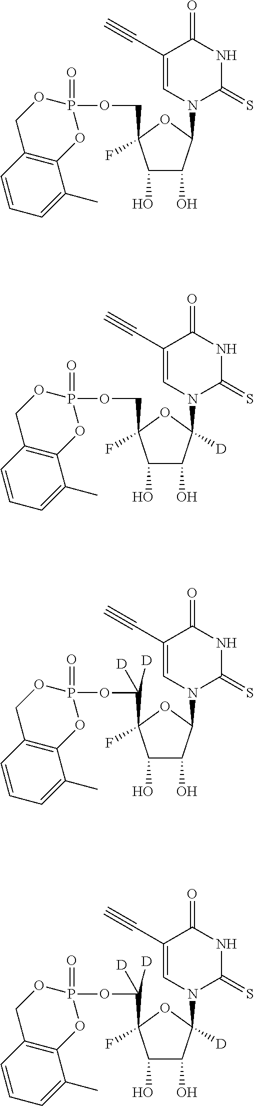 [SMILES: C#Cc1cn([C@@H]2O[C@](F)(COP3(=O)OCc4cccc(C)c4O3)[C@@H](O)[C@H]2O)c(=S)[nH]c1=O ; [2H][C@@]1(n2cc(C#C)c(=O)[nH]c2=S)O[C@](F)(COP2(=O)OCc3cccc(C)c3O2)[C@@H](O)[C@H]1O ; [2H]C([2H])(OP1(=O)OCc2cccc(C)c2O1)[C@@]1(F)O[C@@H](n2cc(C#C)c(=O)[nH]c2=S)[C@H](O)[C@@H]1O ; [2H]C([2H])(OP1(=O)OCc2cccc(C)c2O1)[C@@]1(F)O[C@@]([2H])(n2cc(C#C)c(=O)[nH]c2=S)[C@H](O)[C@@H]1O]

In exemplary embodiments, the compound is selected from:

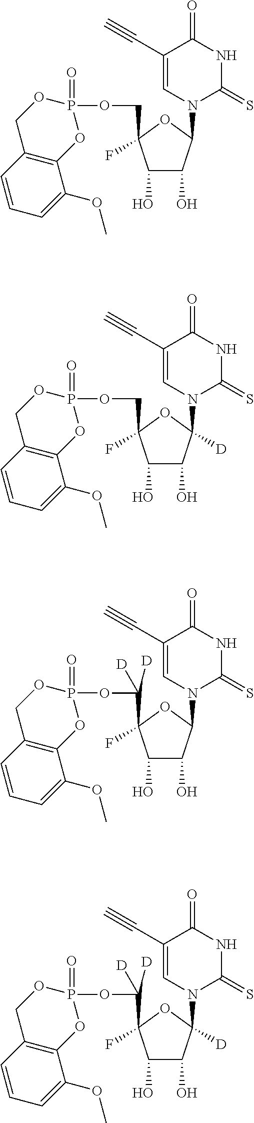 [SMILES: C#Cc1cn([C@@H]2O[C@](F)(COP3(=O)OCc4cccc(OC)c4O3)[C@@H](O)[C@H]2O)c(=S)[nH]c1=O ; [2H][C@@]1(n2cc(C#C)c(=O)[nH]c2=S)O[C@](F)(COP2(=O)OCc3cccc(OC)c3O2)[C@@H](O)[C@H]1O ; [2H]C([2H])(OP1(=O)OCc2cccc(OC)c2O1)[C@@]1(F)O[C@@H](n2cc(C#C)c(=O)[nH]c2=S)[C@H](O)[C@@H]1O ; [2H]C([2H])(OP1(=O)OCc2cccc(OC)c2O1)[C@@]1(F)O[C@@]([2H])(n2cc(C#C)c(=O)[nH]c2=S)[C@H](O)[C@@H]1O]

In exemplary embodiments, the compound is selected from:

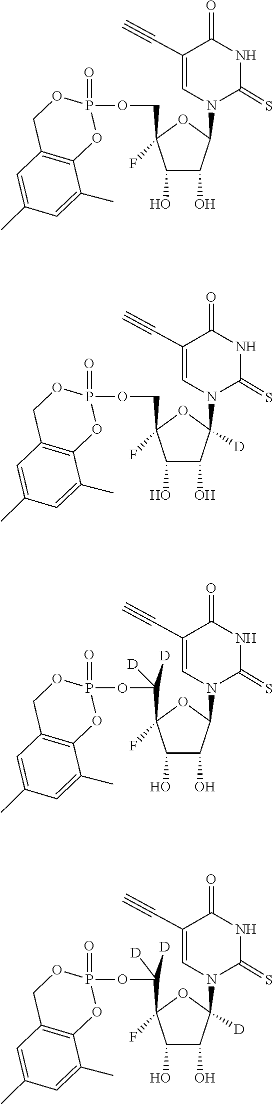

In exemplary embodiments, the compound is selected from:

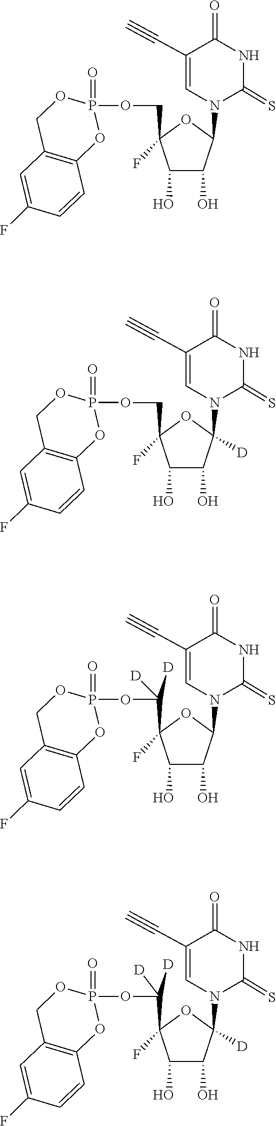

In exemplary embodiments, the compound is selected from:

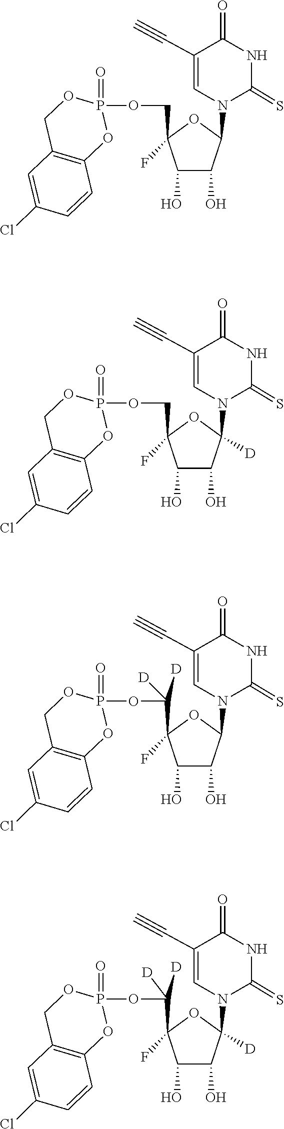

In exemplary embodiments, the compound is selected from:

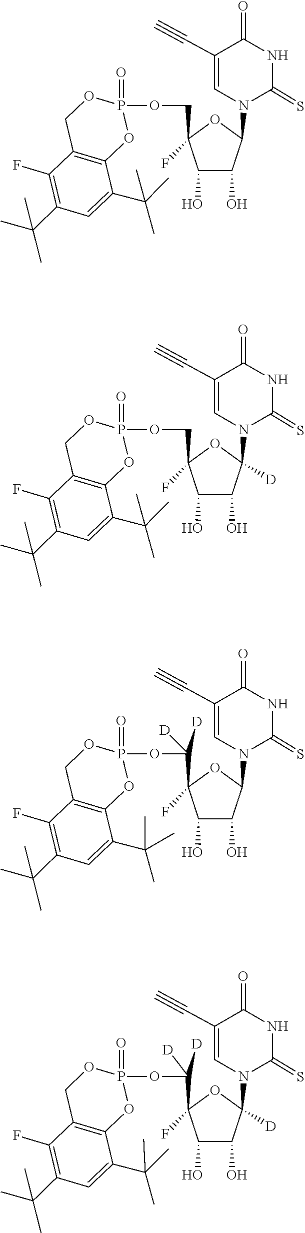

In exemplary embodiments, the compound is selected from:

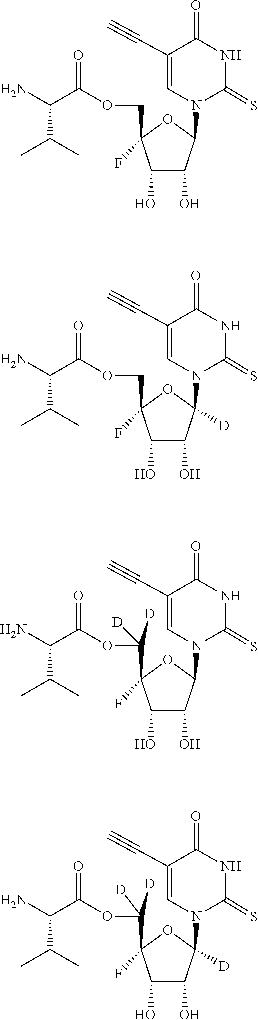

In exemplary embodiments, the compound is selected from:

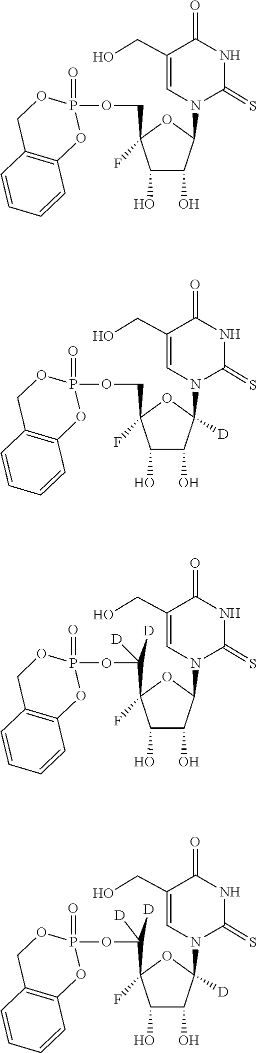

In exemplary embodiments, the compound is selected from:

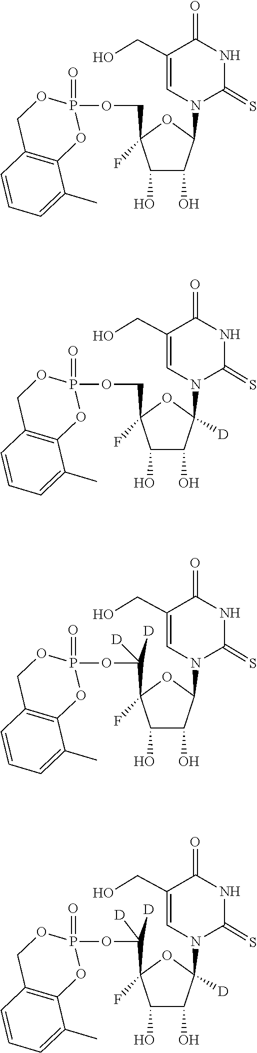

In exemplary embodiments, the compound is selected from:

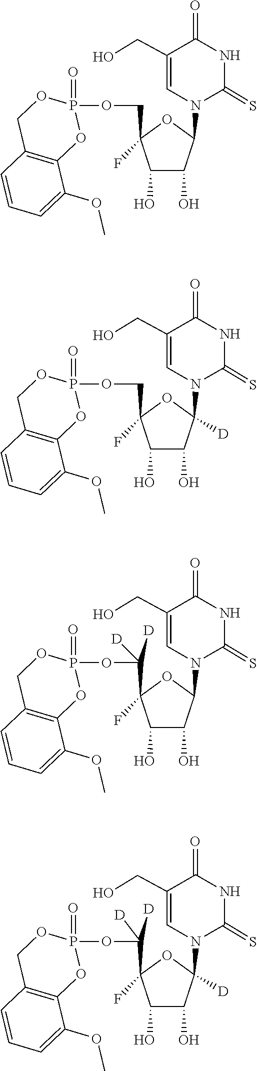

In exemplary embodiments, the compound is selected from:

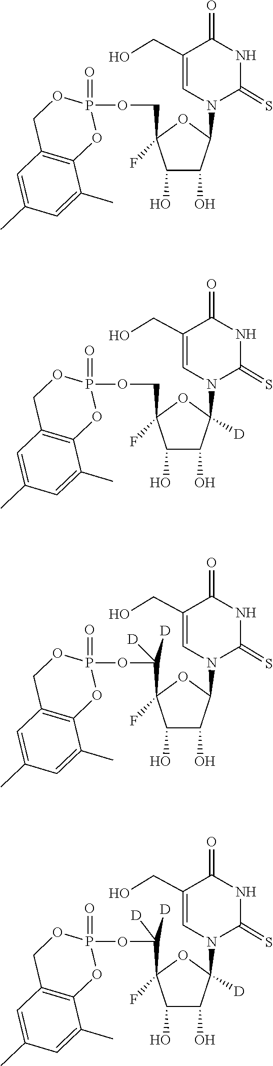

In exemplar embodiments, the compound is selected from:

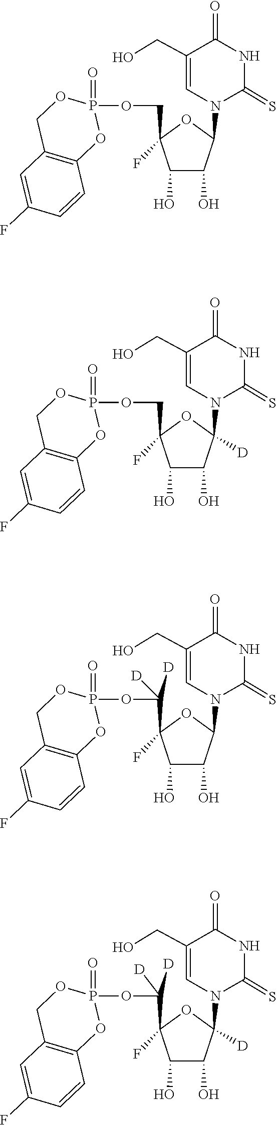

In exemplary embodiments, the compound is selected from:

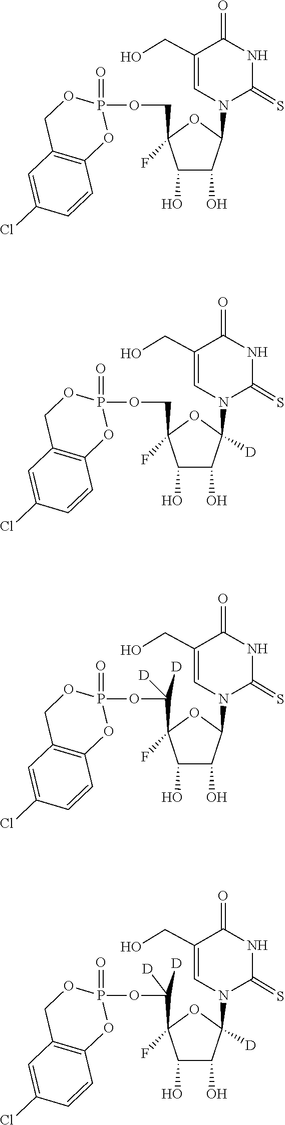

In exemplary embodiments, the compound is selected from:

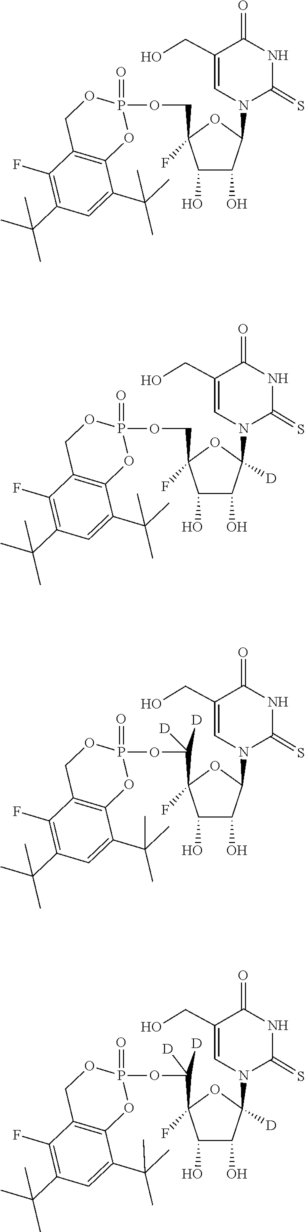

In exemplary embodiments, the compound is selected from:

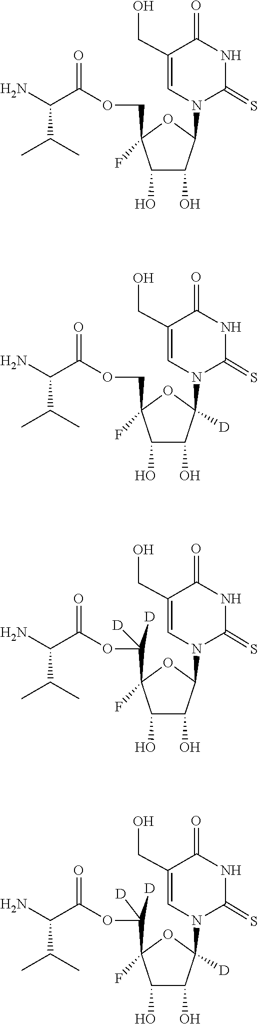

In exemplary embodiments, the compound is selected from:

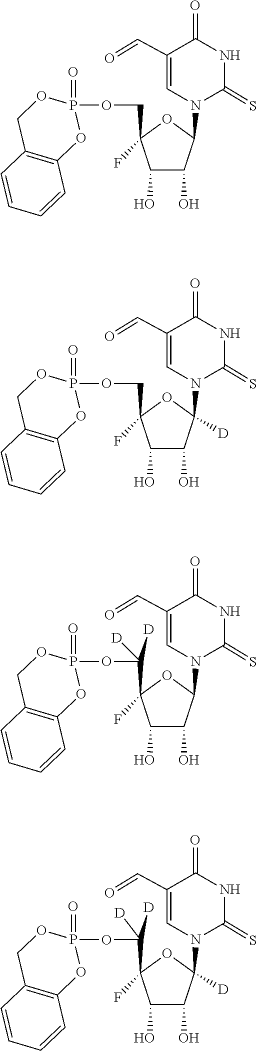

In exemplary embodiments, the compound is selected from:

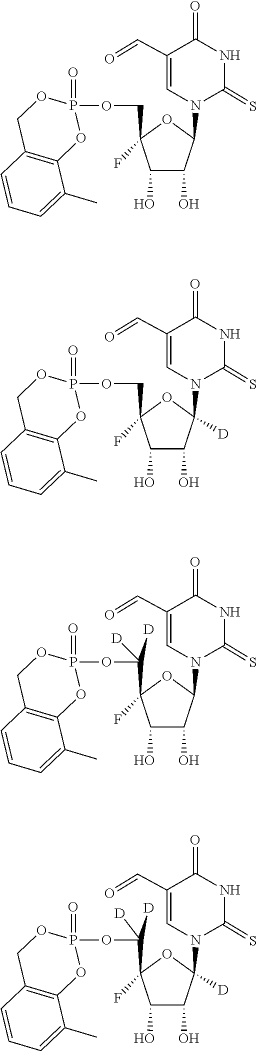

In exemplary embodiments, the compound is selected from:

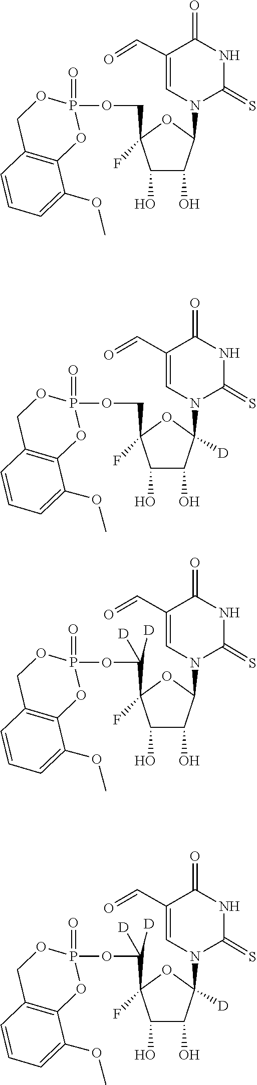 [SMILES: COc1cccc2c1OP(=O)(OC[C@@]1(F)O[C@@H](n3cc(C=O)c(=O)[nH]c3=S)[C@H](O)[C@@H]1O)OC2 ; [2H][C@@]1(n2cc(C=O)c(=O)[nH]c2=S)O[C@](F)(COP2(=O)OCc3cccc(OC)c3O2)[C@@H](O)[C@H]1O ; [2H]C([2H])(OP1(=O)OCc2cccc(OC)c2O1)[C@@]1(F)O[C@@H](n2cc(C=O)c(=O)[nH]c2=S)[C@H](O)[C@@H]1O ; [2H]C([2H])(OP1(=O)OCc2cccc(OC)c2O1)[C@@]1(F)O[C@@]([2H])(n2cc(C=O)c(=O)[nH]c2=S)[C@H](O)[C@@H]1O]

In exemplary embodiments, the compound is selected from:

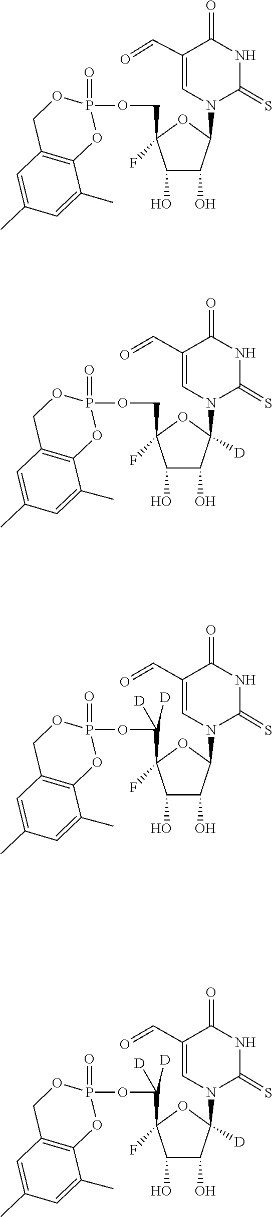 [SMILES: Cc1cc(C)c2c(c1)COP(=O)(OC[C@@]1(F)O[C@@H](n3cc(C=O)c(=O)[nH]c3=S)[C@H](O)[C@@H]1O)O2 ; [2H][C@@]1(n2cc(C=O)c(=O)[nH]c2=S)O[C@](F)(COP2(=O)OCc3cc(C)cc(C)c3O2)[C@@H](O)[C@H]1O ; [2H]C([2H])(OP1(=O)OCc2cc(C)cc(C)c2O1)[C@@]1(F)O[C@@H](n2cc(C=O)c(=O)[nH]c2=S)[C@H](O)[C@@H]1O ; [2H]C([2H])(OP1(=O)OCc2cc(C)cc(C)c2O1)[C@@]1(F)O[C@@]([2H])(n2cc(C=O)c(=O)[nH]c2=S)[C@H](O)[C@@H]1O]

In exemplary embodiments, the compound is selected from:

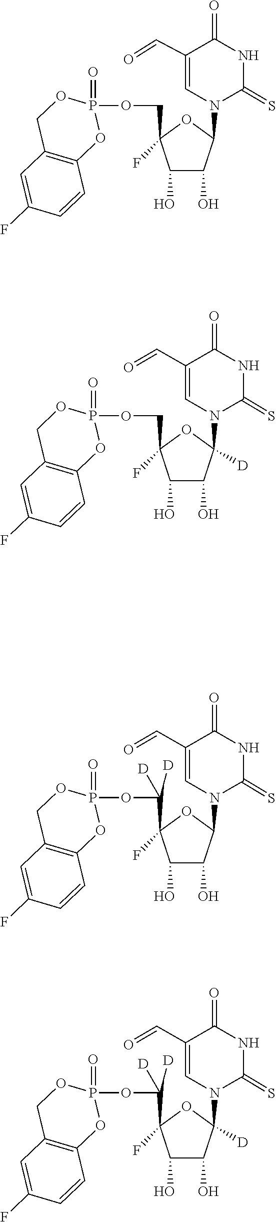 [SMILES: O=Cc1cn([C@@H]2O[C@](F)(COP3(=O)OCc4cc(F)ccc4O3)[C@@H](O)[C@H]2O)c(=S)[nH]c1=O ; [2H][C@@]1(n2cc(C=O)c(=O)[nH]c2=S)O[C@](F)(COP2(=O)OCc3cc(F)ccc3O2)[C@@H](O)[C@H]1O ; [2H]C([2H])(OP1(=O)OCc2cc(F)ccc2O1)[C@@]1(F)O[C@@H](n2cc(C=O)c(=O)[nH]c2=S)[C@H](O)[C@@H]1O ; [2H]C([2H])(OP1(=O)OCc2cc(F)ccc2O1)[C@@]1(F)O[C@@]([2H])(n2cc(C=O)c(=O)[nH]c2=S)[C@H](O)[C@@H]1O]

In exemplary embodiments, the compound is selected from:

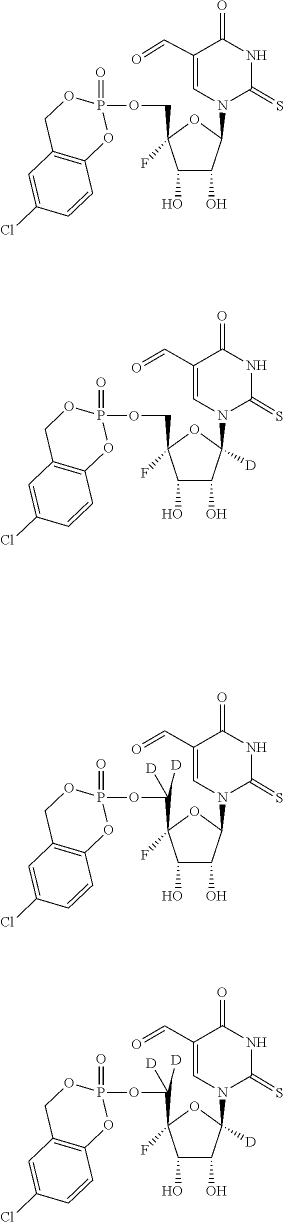 [SMILES: O=Cc1cn([C@@H]2O[C@](F)(COP3(=O)OCc4cc(Cl)ccc4O3)[C@@H](O)[C@H]2O)c(=S)[nH]c1=O ; [2H][C@@]1(n2cc(C=O)c(=O)[nH]c2=S)O[C@](F)(COP2(=O)OCc3cc(Cl)ccc3O2)[C@@H](O)[C@H]1O ; [2H]C([2H])(OP1(=O)OCc2cc(Cl)ccc2O1)[C@@]1(F)O[C@@H](n2cc(C=O)c(=O)[nH]c2=S)[C@H](O)[C@@H]1O ; [2H]C([2H])(OP1(=O)OCc2cc(Cl)ccc2O1)[C@@]1(F)O[C@@]([2H])(n2cc(C=O)c(=O)[nH]c2=S)[C@H](O)[C@@H]1O]

In exemplary embodiments, the compound is selected from:

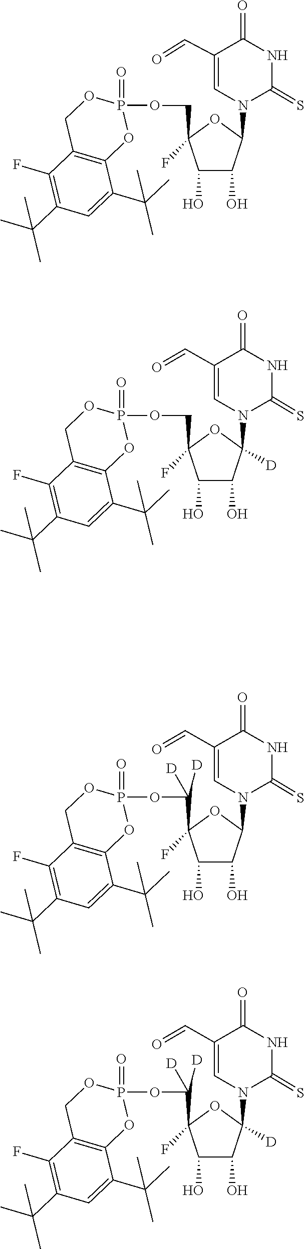 [SMILES: CC(C)(C)c1cc(C(C)(C)C)c2c(c1F)COP(=O)(OC[C@@]1(F)O[C@@H](n3cc(C=O)c(=O)[nH]c3=S)[C@H](O)[C@@H]1O)O2 ; [2H][C@@]1(n2cc(C=O)c(=O)[nH]c2=S)O[C@](F)(COP2(=O)OCc3c(F)c(C(C)(C)C)cc(C(C)(C)C)c3O2)[C@@H](O)[C@H]1O ; [2H]C([2H])(OP1(=O)OCc2c(F)c(C(C)(C)C)cc(C(C)(C)C)c2O1)[C@@]1(F)O[C@@H](n2cc(C=O)c(=O)[nH]c2=S)[C@H](O)[C@@H]1O ; [2H]C([2H])(OP1(=O)OCc2c(F)c(C(C)(C)C)cc(C(C)(C)C)c2O1)[C@@]1(F)O[C@@]([2H])(n2cc(C=O)c(=O)[nH]c2=S)[C@H](O)[C@@H]1O]

In exemplary embodiments, the compound is selected from:

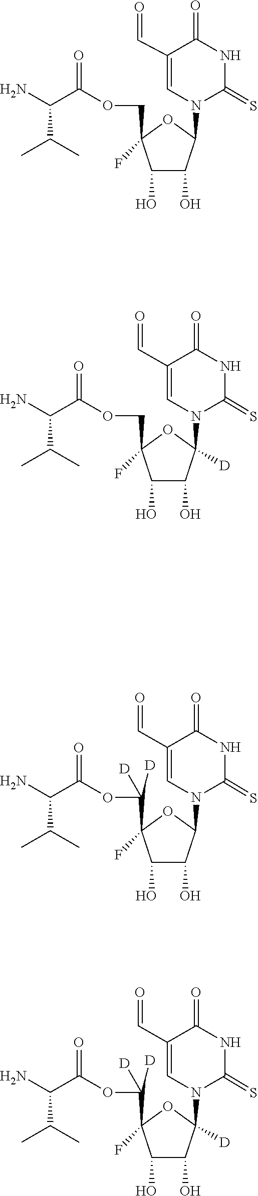 [SMILES: CC(C)[C@H](N)C(=O)OC[C@@]1(F)O[C@@H](n2cc(C=O)c(=O)[nH]c2=S)[C@H](O)[C@@H]1O ; [2H][C@@]1(n2cc(C=O)c(=O)[nH]c2=S)O[C@](F)(COC(=O)[C@@H](N)C(C)C)[C@@H](O)[C@H]1O ; [2H]C([2H])(OC(=O)[C@@H](N)C(C)C)[C@@]1(F)O[C@@H](n2cc(C=O)c(=O)[nH]c2=S)[C@H](O)[C@@H]1O ; [2H]C([2H])(OC(=O)[C@@H](N)C(C)C)[C@@]1(F)O[C@@]([2H])(n2cc(C=O)c(=O)[nH]c2=S)[C@H](O)[C@@H]1O]

In exemplary embodiments, the compound is selected from:

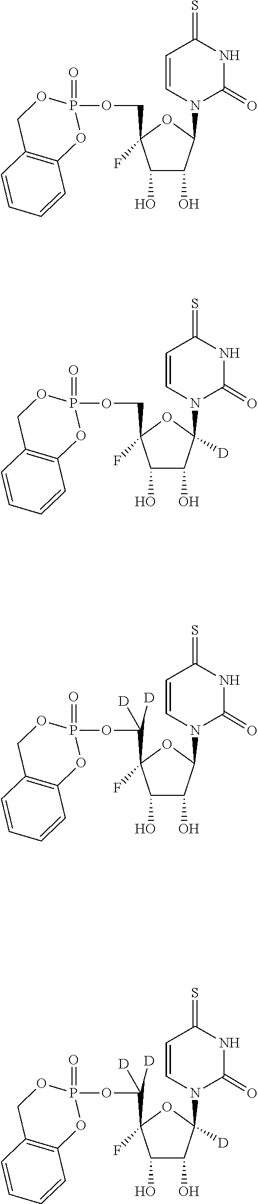 [SMILES: O=c1[nH]c(=S)ccn1[C@@H]1O[C@](F)(COP2(=O)OCc3ccccc3O2)[C@@H](O)[C@H]1O ; [2H][C@@]1(n2ccc(=S)[nH]c2=O)O[C@](F)(COP2(=O)OCc3ccccc3O2)[C@@H](O)[C@H]1O ; [2H]C([2H])(OP1(=O)OCc2ccccc2O1)[C@@]1(F)O[C@@H](n2ccc(=S)[nH]c2=O)[C@H](O)[C@@H]1O ; [2H]C([2H])(OP1(=O)OCc2ccccc2O1)[C@@]1(F)O[C@@]([2H])(n2ccc(=S)[nH]c2=O)[C@H](O)[C@@H]1O]

In exemplary embodiments, the compound is selected from:

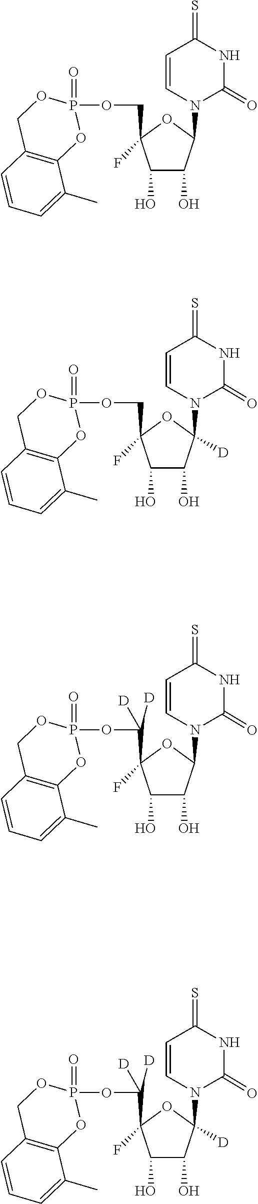 [SMILES: Cc1cccc2c1OP(=O)(OC[C@@]1(F)O[C@@H](n3ccc(=S)[nH]c3=O)[C@H](O)[C@@H]1O)OC2 ; [2H][C@@]1(n2ccc(=S)[nH]c2=O)O[C@](F)(COP2(=O)OCc3cccc(C)c3O2)[C@@H](O)[C@H]1O ; [2H]C([2H])(OP1(=O)OCc2cccc(C)c2O1)[C@@]1(F)O[C@@H](n2ccc(=S)[nH]c2=O)[C@H](O)[C@@H]1O ; [2H]C([2H])(OP1(=O)OCc2cccc(C)c2O1)[C@@]1(F)O[C@@]([2H])(n2ccc(=S)[nH]c2=O)[C@H](O)[C@@H]1O]

In exemplary embodiments, the compound is selected from:

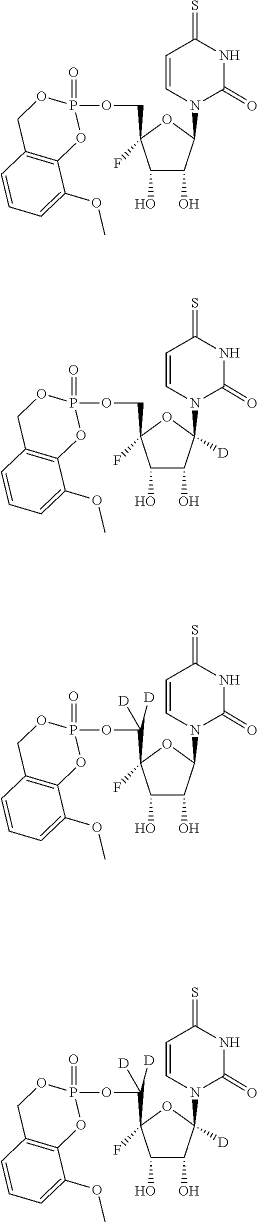 [SMILES: COc1cccc2c1OP(=O)(OC[C@@]1(F)O[C@@H](n3ccc(=S)[nH]c3=O)[C@H](O)[C@@H]1O)OC2 ; [2H][C@@]1(n2ccc(=S)[nH]c2=O)O[C@](F)(COP2(=O)OCc3cccc(OC)c3O2)[C@@H](O)[C@H]1O ; [2H]C([2H])(OP1(=O)OCc2cccc(OC)c2O1)[C@@]1(F)O[C@@H](n2ccc(=S)[nH]c2=O)[C@H](O)[C@@H]1O ; [2H]C([2H])(OP1(=O)OCc2cccc(OC)c2O1)[C@@]1(F)O[C@@]([2H])(n2ccc(=S)[nH]c2=O)[C@H](O)[C@@H]1O]

In exemplary embodiments, the compound is selected from:

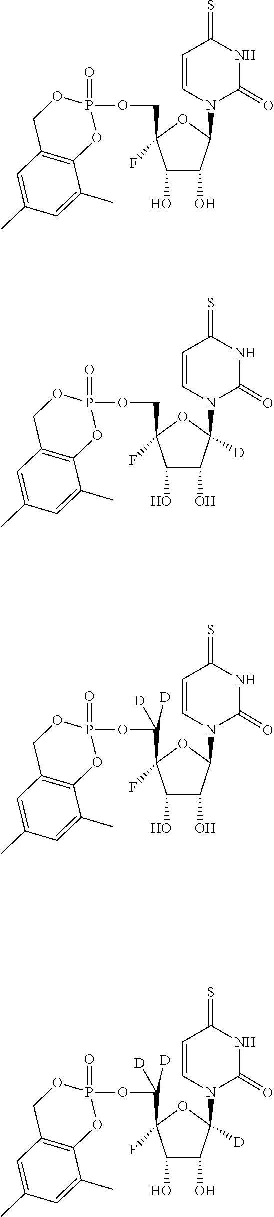 [SMILES: Cc1cc(C)c2c(c1)COP(=O)(OC[C@@]1(F)O[C@@H](n3ccc(=S)[nH]c3=O)[C@H](O)[C@@H]1O)O2 ; [2H][C@@]1(n2ccc(=S)[nH]c2=O)O[C@](F)(COP2(=O)OCc3cc(C)cc(C)c3O2)[C@@H](O)[C@H]1O ; [2H]C([2H])(OP1(=O)OCc2cc(C)cc(C)c2O1)[C@@]1(F)O[C@@H](n2ccc(=S)[nH]c2=O)[C@H](O)[C@@H]1O ; [2H]C([2H])(OP1(=O)OCc2cc(C)cc(C)c2O1)[C@@]1(F)O[C@@]([2H])(n2ccc(=S)[nH]c2=O)[C@H](O)[C@@H]1O]

In exemplary embodiments, the compound is selected from:

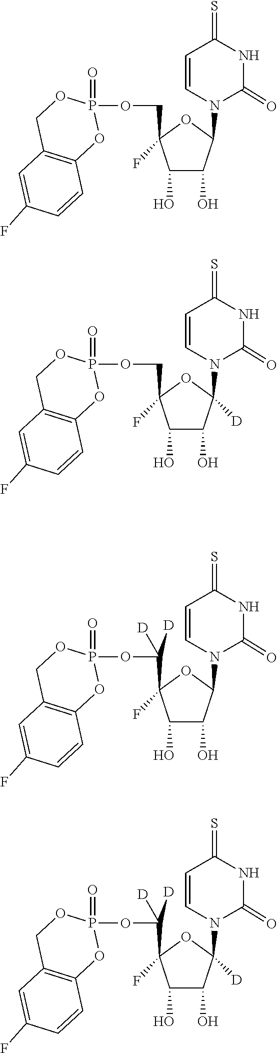

In exemplary embodiments, the compound is selected from:

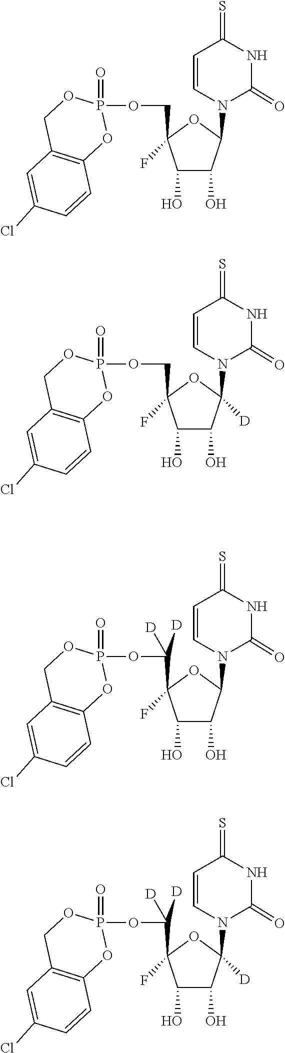

In exemplary embodiments, the compound is selected from:

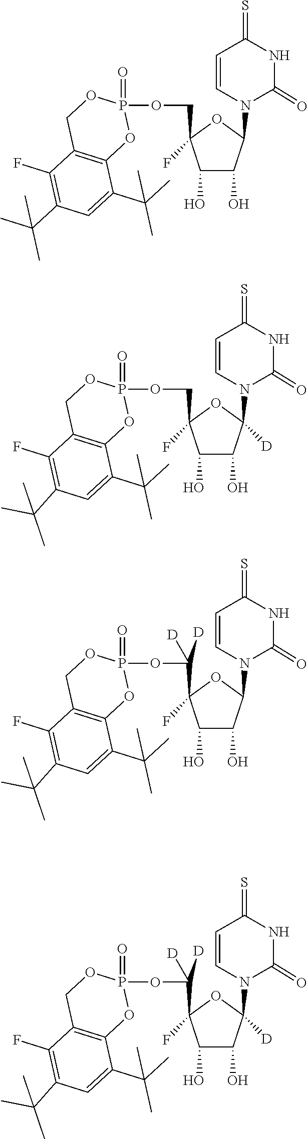

In exemplary embodiments, the compound is selected from:

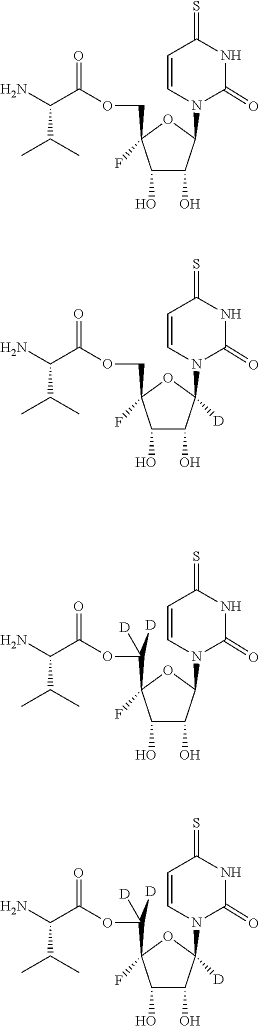

In exemplary embodiments, the compound is selected from:

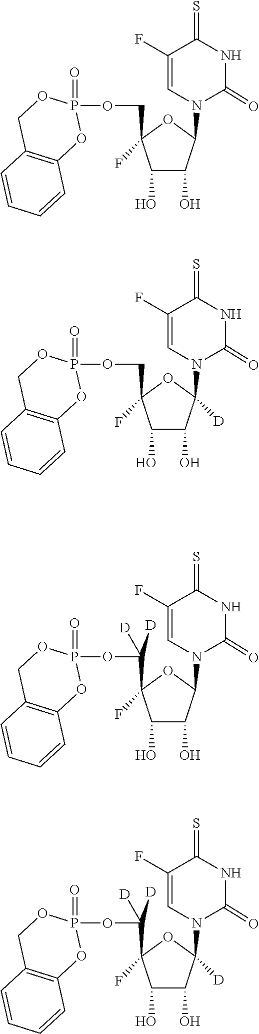

In exemplary embodiments, the compound is selected from:

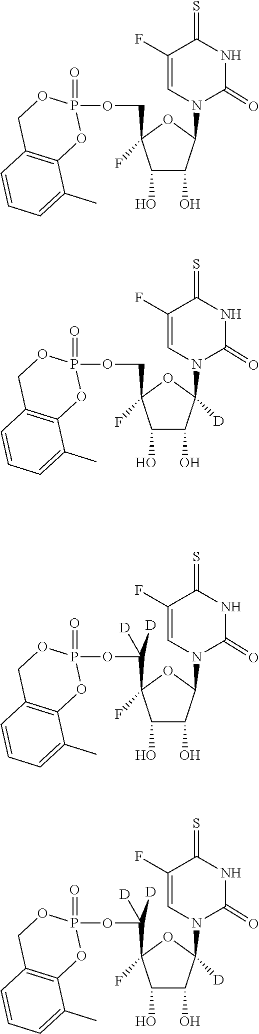

In exemplary embodiments, the compound is selected from:

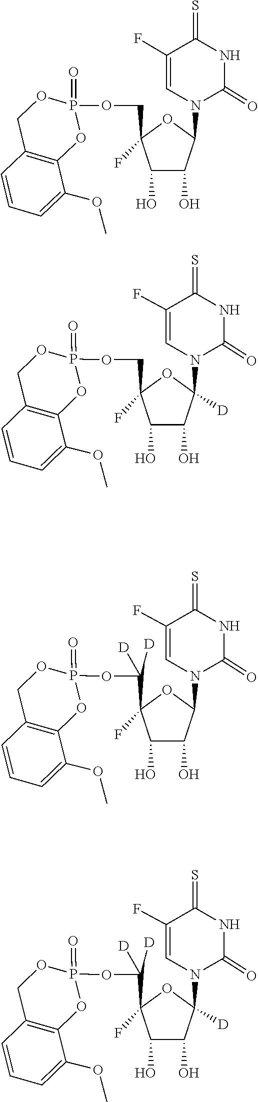 [SMILES: COc1cccc2c1OP(=O)(OC[C@@]1(F)O[C@@H](n3cc(F)c(=S)[nH]c3=O)[C@H](O)[C@@H]1O)OC2 ; [2H][C@@]1(n2cc(F)c(=S)[nH]c2=O)O[C@](F)(COP2(=O)OCc3cccc(OC)c3O2)[C@@H](O)[C@H]1O ; [2H]C([2H])(OP1(=O)OCc2cccc(OC)c2O1)[C@@]1(F)O[C@@H](n2cc(F)c(=S)[nH]c2=O)[C@H](O)[C@@H]1O ; [2H]C([2H])(OP1(=O)OCc2cccc(OC)c2O1)[C@@]1(F)O[C@@]([2H])(n2cc(F)c(=S)[nH]c2=O)[C@H](O)[C@@H]1O]

In exemplary embodiments, the compound is selected from:

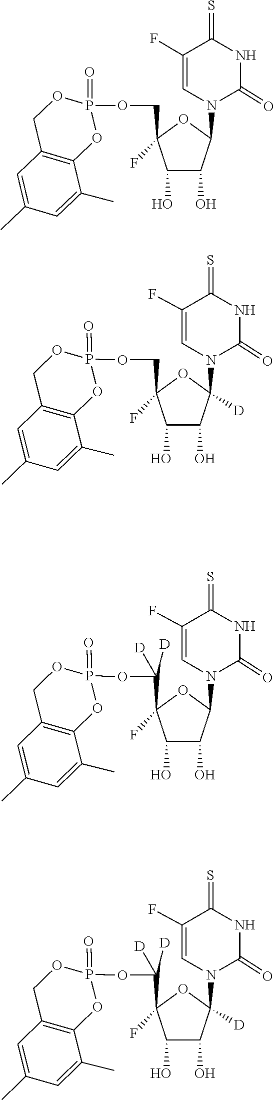 [SMILES: Cc1cc(C)c2c(c1)COP(=O)(OC[C@@]1(F)O[C@@H](n3cc(F)c(=S)[nH]c3=O)[C@H](O)[C@@H]1O)O2 ; [2H][C@@]1(n2cc(F)c(=S)[nH]c2=O)O[C@](F)(COP2(=O)OCc3cc(C)cc(C)c3O2)[C@@H](O)[C@H]1O ; [2H]C([2H])(OP1(=O)OCc2cc(C)cc(C)c2O1)[C@@]1(F)O[C@@H](n2cc(F)c(=S)[nH]c2=O)[C@H](O)[C@@H]1O ; [2H]C([2H])(OP1(=O)OCc2cc(C)cc(C)c2O1)[C@@]1(F)O[C@@]([2H])(n2cc(F)c(=S)[nH]c2=O)[C@H](O)[C@@H]1O]

In exemplary embodiments, the compound is selected from:

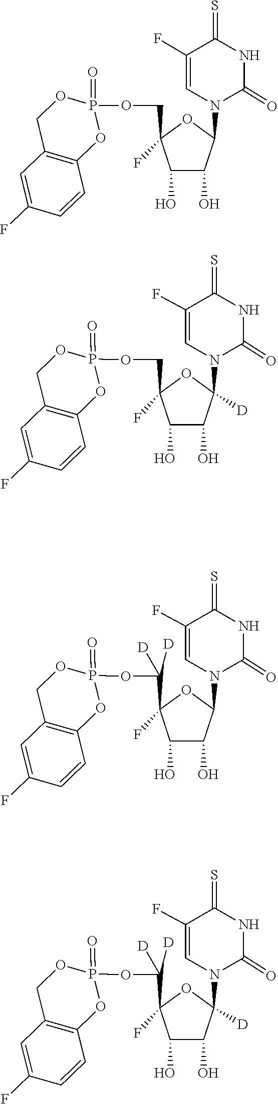 [SMILES: O=c1[nH]c(=S)c(F)cn1[C@@H]1O[C@](F)(COP2(=O)OCc3cc(F)ccc3O2)[C@@H](O)[C@H]1O ; [2H][C@@]1(n2cc(F)c(=S)[nH]c2=O)O[C@](F)(COP2(=O)OCc3cc(F)ccc3O2)[C@@H](O)[C@H]1O ; [2H]C([2H])(OP1(=O)OCc2cc(F)ccc2O1)[C@@]1(F)O[C@@H](n2cc(F)c(=S)[nH]c2=O)[C@H](O)[C@@H]1O ; [2H]C([2H])(OP1(=O)OCc2cc(F)ccc2O1)[C@@]1(F)O[C@@]([2H])(n2cc(F)c(=S)[nH]c2=O)[C@H](O)[C@@H]1O]

In exemplary embodiments, the compound is selected from:

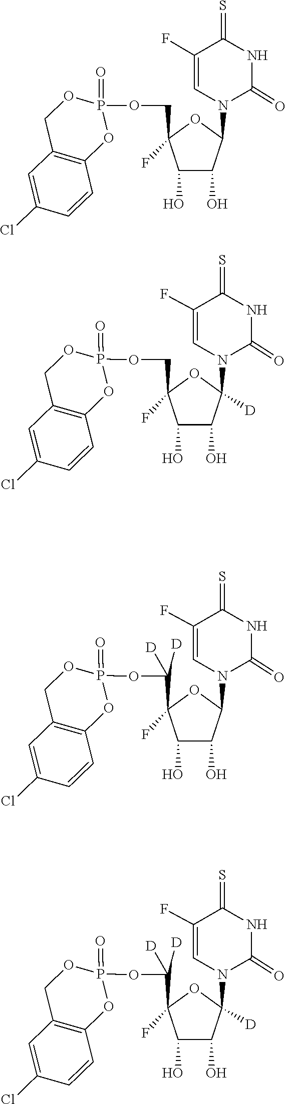 [SMILES: O=c1[nH]c(=S)c(F)cn1[C@@H]1O[C@](F)(COP2(=O)OCc3cc(Cl)ccc3O2)[C@@H](O)[C@H]1O ; [2H][C@@]1(n2cc(F)c(=S)[nH]c2=O)O[C@](F)(COP2(=O)OCc3cc(Cl)ccc3O2)[C@@H](O)[C@H]1O ; [2H]C([2H])(OP1(=O)OCc2cc(Cl)ccc2O1)[C@@]1(F)O[C@@H](n2cc(F)c(=S)[nH]c2=O)[C@H](O)[C@@H]1O ; [2H]C([2H])(OP1(=O)OCc2cc(Cl)ccc2O1)[C@@]1(F)O[C@@]([2H])(n2cc(F)c(=S)[nH]c2=O)[C@H](O)[C@@H]1O]

In exemplary embodiments, the compound is selected from:

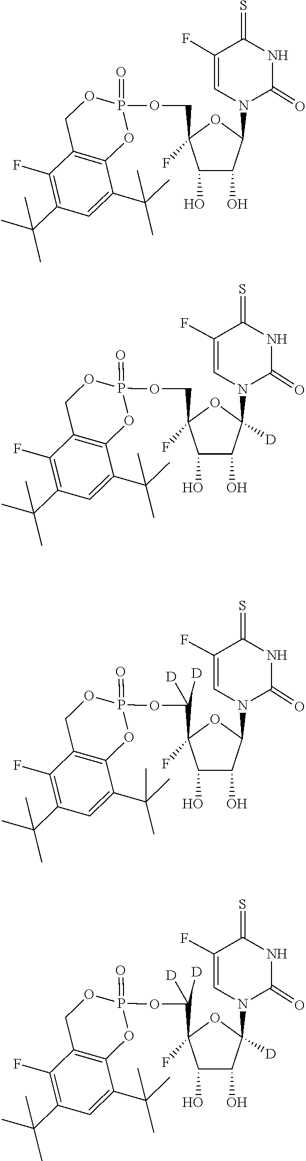 [SMILES: CC(C)(C)c1cc(C(C)(C)C)c2c(c1F)COP(=O)(OC[C@@]1(F)O[C@@H](n3cc(F)c(=S)[nH]c3=O)[C@H](O)[C@@H]1O)O2 ; [2H][C@@]1(n2cc(F)c(=S)[nH]c2=O)O[C@](F)(COP2(=O)OCc3c(F)c(C(C)(C)C)cc(C(C)(C)C)c3O2)[C@@H](O)[C@H]1O ; [2H]C([2H])(OP1(=O)OCc2c(F)c(C(C)(C)C)cc(C(C)(C)C)c2O1)[C@@]1(F)O[C@@H](n2cc(F)c(=S)[nH]c2=O)[C@H](O)[C@@H]1O ; [2H]C([2H])(OP1(=O)OCc2c(F)c(C(C)(C)C)cc(C(C)(C)C)c2O1)[C@@]1(F)O[C@@]([2H])(n2cc(F)c(=S)[nH]c2=O)[C@H](O)[C@@H]1O]

In exemplary embodiments, the compound is selected from:

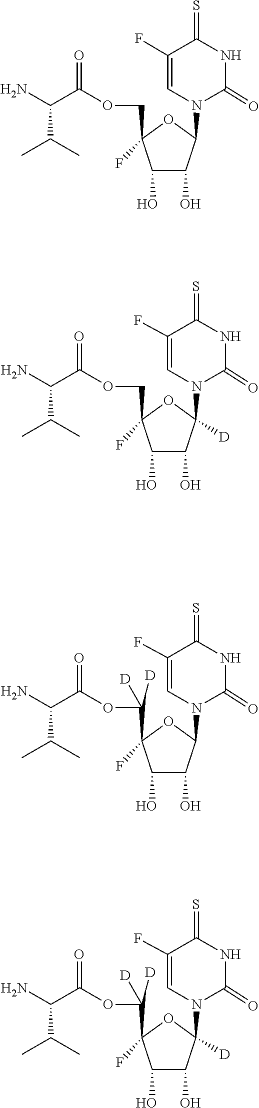 [SMILES: CC(C)[C@H](N)C(=O)OC[C@@]1(F)O[C@@H](n2cc(F)c(=S)[nH]c2=O)[C@H](O)[C@@H]1O ; [2H][C@@]1(n2cc(F)c(=S)[nH]c2=O)O[C@](F)(COC(=O)[C@@H](N)C(C)C)[C@@H](O)[C@H]1O ; [2H]C([2H])(OC(=O)[C@@H](N)C(C)C)[C@@]1(F)O[C@@H](n2cc(F)c(=S)[nH]c2=O)[C@H](O)[C@@H]1O ; [2H]C([2H])(OC(=O)[C@@H](N)C(C)C)[C@@]1(F)O[C@@]([2H])(n2cc(F)c(=S)[nH]c2=O)[C@H](O)[C@@H]1O]

In exemplary embodiments, the compound is selected from:

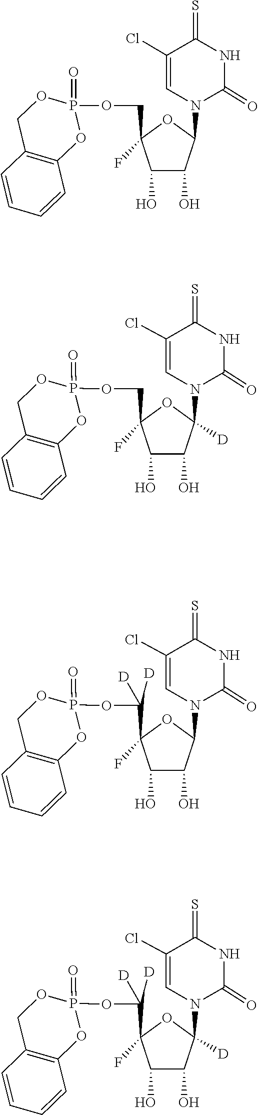 [SMILES: O=c1[nH]c(=S)c(Cl)cn1[C@@H]1O[C@](F)(COP2(=O)OCc3ccccc3O2)[C@@H](O)[C@H]1O ; [2H][C@@]1(n2cc(Cl)c(=S)[nH]c2=O)O[C@](F)(COP2(=O)OCc3ccccc3O2)[C@@H](O)[C@H]1O ; [2H]C([2H])(OP1(=O)OCc2ccccc2O1)[C@@]1(F)O[C@@H](n2cc(Cl)c(=S)[nH]c2=O)[C@H](O)[C@@H]1O ; [2H]C([2H])(OP1(=O)OCc2ccccc2O1)[C@@]1(F)O[C@@]([2H])(n2cc(Cl)c(=S)[nH]c2=O)[C@H](O)[C@@H]1O]

In exemplary embodiments, the compound is selected from:

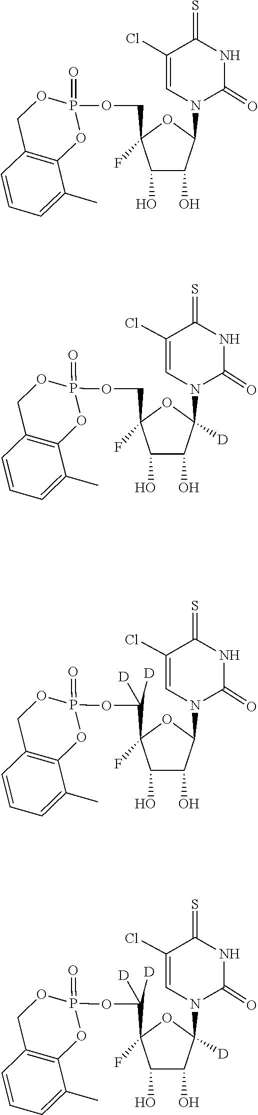 [SMILES: Cc1cccc2c1OP(=O)(OC[C@@]1(F)O[C@@H](n3cc(Cl)c(=S)[nH]c3=O)[C@H](O)[C@@H]1O)OC2 ; [2H][C@@]1(n2cc(Cl)c(=S)[nH]c2=O)O[C@](F)(COP2(=O)OCc3cccc(C)c3O2)[C@@H](O)[C@H]1O ; [2H]C([2H])(OP1(=O)OCc2cccc(C)c2O1)[C@@]1(F)O[C@@H](n2cc(Cl)c(=S)[nH]c2=O)[C@H](O)[C@@H]1O ; [2H]C([2H])(OP1(=O)OCc2cccc(C)c2O1)[C@@]1(F)O[C@@]([2H])(n2cc(Cl)c(=S)[nH]c2=O)[C@H](O)[C@@H]1O]

In exemplary embodiments, the compound is selected from:

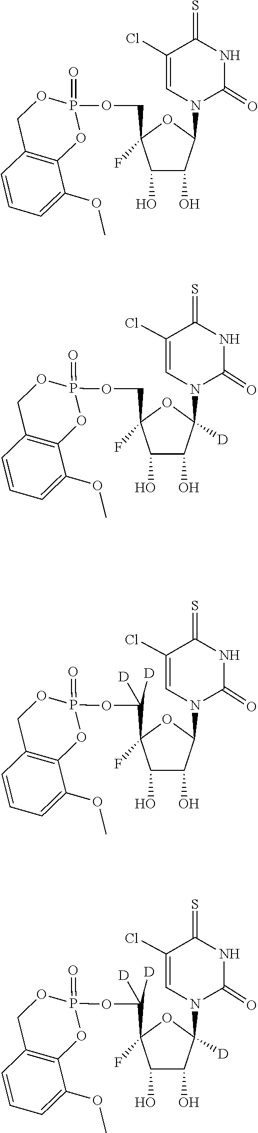 [SMILES: COc1cccc2c1OP(=O)(OC[C@@]1(F)O[C@@H](n3cc(Cl)c(=S)[nH]c3=O)[C@H](O)[C@@H]1O)OC2 ; [2H][C@@]1(n2cc(Cl)c(=S)[nH]c2=O)O[C@](F)(COP2(=O)OCc3cccc(OC)c3O2)[C@@H](O)[C@H]1O ; [2H]C([2H])(OP1(=O)OCc2cccc(OC)c2O1)[C@@]1(F)O[C@@H](n2cc(Cl)c(=S)[nH]c2=O)[C@H](O)[C@@H]1O ; [2H]C([2H])(OP1(=O)OCc2cccc(OC)c2O1)[C@@]1(F)O[C@@]([2H])(n2cc(Cl)c(=S)[nH]c2=O)[C@H](O)[C@@H]1O]

In exemplary embodiments, the compound is selected from:

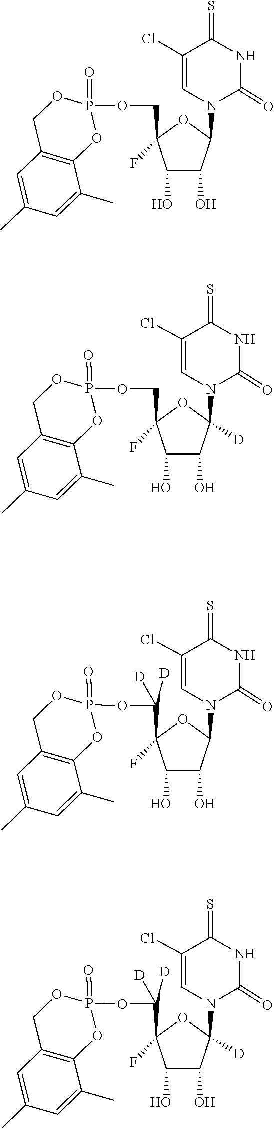 [SMILES: Cc1cc(C)c2c(c1)COP(=O)(OC[C@@]1(F)O[C@@H](n3cc(Cl)c(=S)[nH]c3=O)[C@H](O)[C@@H]1O)O2 ; [2H][C@@]1(n2cc(Cl)c(=S)[nH]c2=O)O[C@](F)(COP2(=O)OCc3cc(C)cc(C)c3O2)[C@@H](O)[C@H]1O ; [2H]C([2H])(OP1(=O)OCc2cc(C)cc(C)c2O1)[C@@]1(F)O[C@@H](n2cc(Cl)c(=S)[nH]c2=O)[C@H](O)[C@@H]1O ; [2H]C([2H])(OP1(=O)OCc2cc(C)cc(C)c2O1)[C@@]1(F)O[C@@]([2H])(n2cc(Cl)c(=S)[nH]c2=O)[C@H](O)[C@@H]1O]

In exemplary embodiments, the compound is selected from:

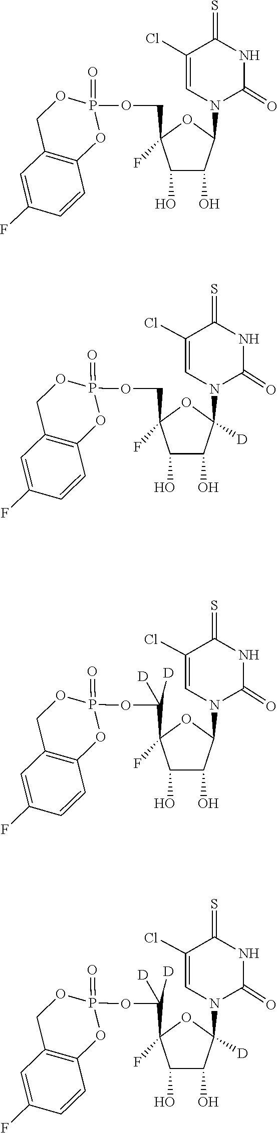 [SMILES: O=c1[nH]c(=S)c(Cl)cn1[C@@H]1O[C@](F)(COP2(=O)OCc3cc(F)ccc3O2)[C@@H](O)[C@H]1O ; [2H][C@@]1(n2cc(Cl)c(=S)[nH]c2=O)O[C@](F)(COP2(=O)OCc3cc(F)ccc3O2)[C@@H](O)[C@H]1O ; [2H]C([2H])(OP1(=O)OCc2cc(F)ccc2O1)[C@@]1(F)O[C@@H](n2cc(Cl)c(=S)[nH]c2=O)[C@H](O)[C@@H]1O ; [2H]C([2H])(OP1(=O)OCc2cc(F)ccc2O1)[C@@]1(F)O[C@@]([2H])(n2cc(Cl)c(=S)[nH]c2=O)[C@H](O)[C@@H]1O]

In exemplary embodiments, the compound is selected from:

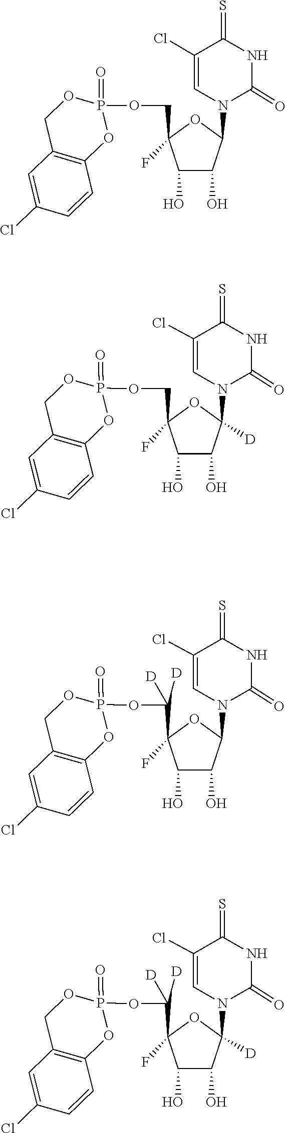 [SMILES: O=c1[nH]c(=S)c(Cl)cn1[C@@H]1O[C@](F)(COP2(=O)OCc3cc(Cl)ccc3O2)[C@@H](O)[C@H]1O ; [2H][C@@]1(n2cc(Cl)c(=S)[nH]c2=O)O[C@](F)(COP2(=O)OCc3cc(Cl)ccc3O2)[C@@H](O)[C@H]1O ; [2H]C([2H])(OP1(=O)OCc2cc(Cl)ccc2O1)[C@@]1(F)O[C@@H](n2cc(Cl)c(=S)[nH]c2=O)[C@H](O)[C@@H]1O ; [2H]C([2H])(OP1(=O)OCc2cc(Cl)ccc2O1)[C@@]1(F)O[C@@]([2H])(n2cc(Cl)c(=S)[nH]c2=O)[C@H](O)[C@@H]1O]

In exemplary embodiments, the compound is selected from:

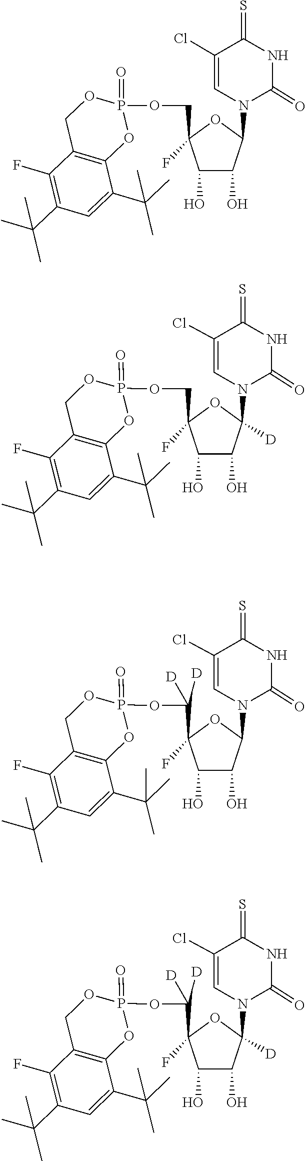 [SMILES: CC(C)(C)c1cc(C(C)(C)C)c2c(c1F)COP(=O)(OC[C@@]1(F)O[C@@H](n3cc(Cl)c(=S)[nH]c3=O)[C@H](O)[C@@H]1O)O2 ; [2H][C@@]1(n2cc(Cl)c(=S)[nH]c2=O)O[C@](F)(COP2(=O)OCc3c(F)c(C(C)(C)C)cc(C(C)(C)C)c3O2)[C@@H](O)[C@H]1O ; [2H]C([2H])(OP1(=O)OCc2c(F)c(C(C)(C)C)cc(C(C)(C)C)c2O1)[C@@]1(F)O[C@@H](n2cc(Cl)c(=S)[nH]c2=O)[C@H](O)[C@@H]1O ; [2H]C([2H])(OP1(=O)OCc2c(F)c(C(C)(C)C)cc(C(C)(C)C)c2O1)[C@@]1(F)O[C@@]([2H])(n2cc(Cl)c(=S)[nH]c2=O)[C@H](O)[C@@H]1O]

In exemplary embodiments, the compound is selected from:

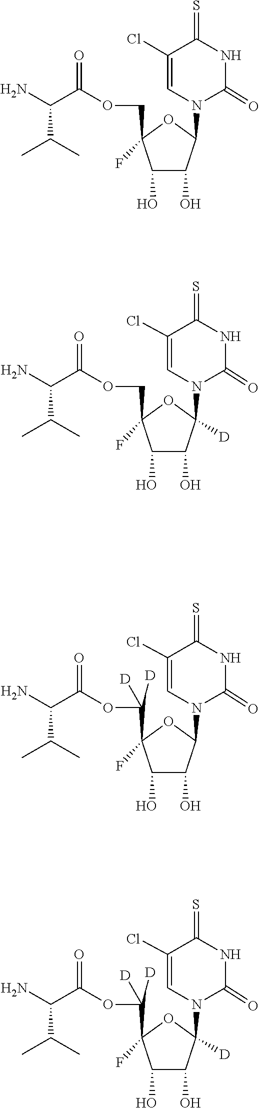 [SMILES: CC(C)[C@H](N)C(=O)OC[C@@]1(F)O[C@@H](n2cc(Cl)c(=S)[nH]c2=O)[C@H](O)[C@@H]1O ; [2H][C@@]1(n2cc(Cl)c(=S)[nH]c2=O)O[C@](F)(COC(=O)[C@@H](N)C(C)C)[C@@H](O)[C@H]1O ; [2H]C([2H])(OC(=O)[C@@H](N)C(C)C)[C@@]1(F)O[C@@H](n2cc(Cl)c(=S)[nH]c2=O)[C@H](O)[C@@H]1O ; [2H]C([2H])(OC(=O)[C@@H](N)C(C)C)[C@@]1(F)O[C@@]([2H])(n2cc(Cl)c(=S)[nH]c2=O)[C@H](O)[C@@H]1O]

In exemplary embodiments, the compound is selected from:

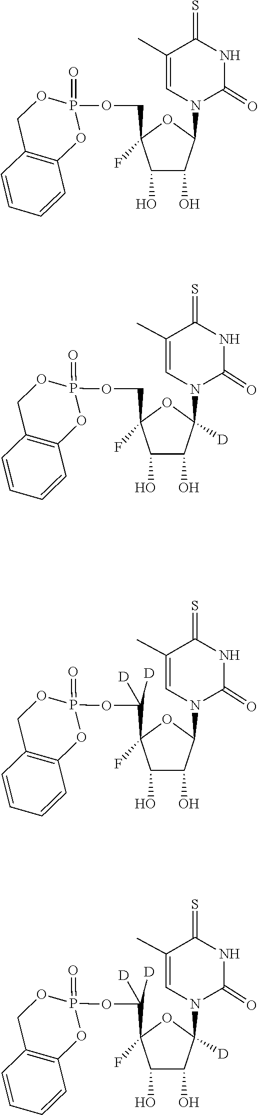 [SMILES: Cc1cn([C@@H]2O[C@](F)(COP3(=O)OCc4ccccc4O3)[C@@H](O)[C@H]2O)c(=O)[nH]c1=S ; [2H][C@@]1(n2cc(C)c(=S)[nH]c2=O)O[C@](F)(COP2(=O)OCc3ccccc3O2)[C@@H](O)[C@H]1O ; [2H]C([2H])(OP1(=O)OCc2ccccc2O1)[C@@]1(F)O[C@@H](n2cc(C)c(=S)[nH]c2=O)[C@H](O)[C@@H]1O ; [2H]C([2H])(OP1(=O)OCc2ccccc2O1)[C@@]1(F)O[C@@]([2H])(n2cc(C)c(=S)[nH]c2=O)[C@H](O)[C@@H]1O]

In exemplary embodiments, the compound is selected from:

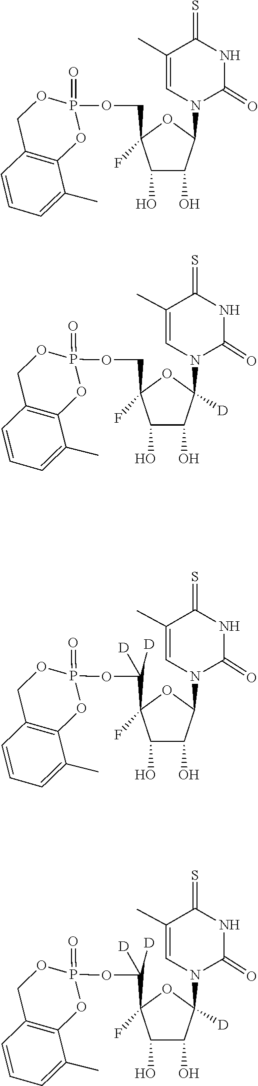 [SMILES: Cc1cccc2c1OP(=O)(OC[C@@]1(F)O[C@@H](n3cc(C)c(=S)[nH]c3=O)[C@H](O)[C@@H]1O)OC2 ; [2H][C@@]1(n2cc(C)c(=S)[nH]c2=O)O[C@](F)(COP2(=O)OCc3cccc(C)c3O2)[C@@H](O)[C@H]1O ; [2H]C([2H])(OP1(=O)OCc2cccc(C)c2O1)[C@@]1(F)O[C@@H](n2cc(C)c(=S)[nH]c2=O)[C@H](O)[C@@H]1O ; [2H]C([2H])(OP1(=O)OCc2cccc(C)c2O1)[C@@]1(F)O[C@@]([2H])(n2cc(C)c(=S)[nH]c2=O)[C@H](O)[C@@H]1O]

In exemplary embodiments, the compound is selected from:

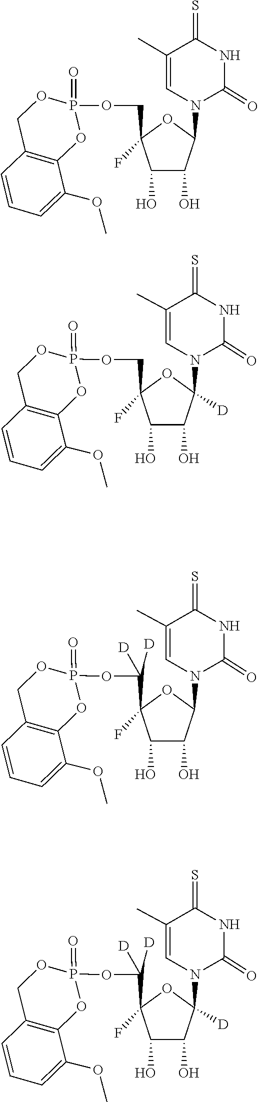 [SMILES: COc1cccc2c1OP(=O)(OC[C@@]1(F)O[C@@H](n3cc(C)c(=S)[nH]c3=O)[C@H](O)[C@@H]1O)OC2 ; [2H][C@@]1(n2cc(C)c(=S)[nH]c2=O)O[C@](F)(COP2(=O)OCc3cccc(OC)c3O2)[C@@H](O)[C@H]1O ; [2H]C([2H])(OP1(=O)OCc2cccc(OC)c2O1)[C@@]1(F)O[C@@H](n2cc(C)c(=S)[nH]c2=O)[C@H](O)[C@@H]1O ; [2H]C([2H])(OP1(=O)OCc2cccc(OC)c2O1)[C@@]1(F)O[C@@]([2H])(n2cc(C)c(=S)[nH]c2=O)[C@H](O)[C@@H]1O]

In exemplary embodiments, the compound is selected from:

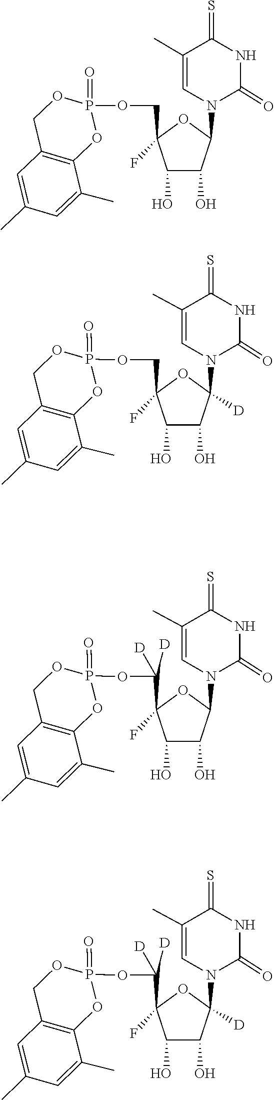 [SMILES: Cc1cc(C)c2c(c1)COP(=O)(OC[C@@]1(F)O[C@@H](n3cc(C)c(=S)[nH]c3=O)[C@H](O)[C@@H]1O)O2 ; [2H][C@@]1(n2cc(C)c(=S)[nH]c2=O)O[C@](F)(COP2(=O)OCc3cc(C)cc(C)c3O2)[C@@H](O)[C@H]1O ; [2H]C([2H])(OP1(=O)OCc2cc(C)cc(C)c2O1)[C@@]1(F)O[C@@H](n2cc(C)c(=S)[nH]c2=O)[C@H](O)[C@@H]1O ; [2H]C([2H])(OP1(=O)OCc2cc(C)cc(C)c2O1)[C@@]1(F)O[C@@]([2H])(n2cc(C)c(=S)[nH]c2=O)[C@H](O)[C@@H]1O]

In exemplary embodiments, the compound is selected from:

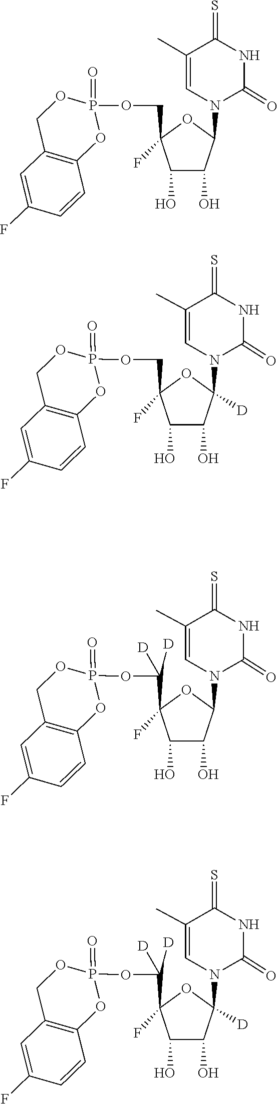 [SMILES: Cc1cn([C@@H]2O[C@](F)(COP3(=O)OCc4cc(F)ccc4O3)[C@@H](O)[C@H]2O)c(=O)[nH]c1=S ; [2H][C@@]1(n2cc(C)c(=S)[nH]c2=O)O[C@](F)(COP2(=O)OCc3cc(F)ccc3O2)[C@@H](O)[C@H]1O ; [2H]C([2H])(OP1(=O)OCc2cc(F)ccc2O1)[C@@]1(F)O[C@@H](n2cc(C)c(=S)[nH]c2=O)[C@H](O)[C@@H]1O ; [2H]C([2H])(OP1(=O)OCc2cc(F)ccc2O1)[C@@]1(F)O[C@@]([2H])(n2cc(C)c(=S)[nH]c2=O)[C@H](O)[C@@H]1O]

In exemplary embodiments, the compound is selected from:

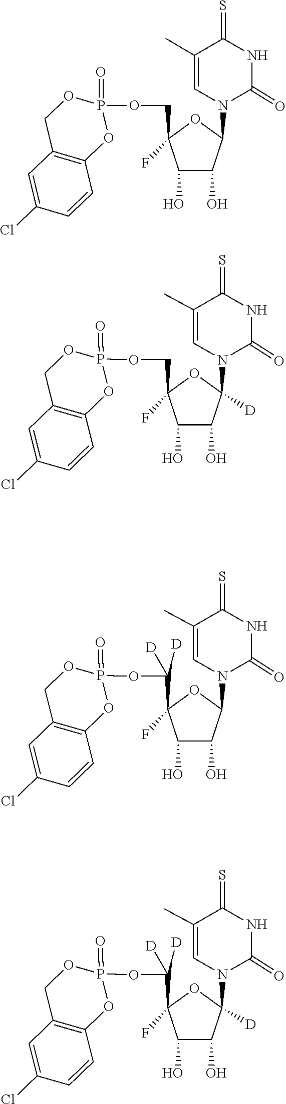 [SMILES: Cc1cn([C@@H]2O[C@](F)(COP3(=O)OCc4cc(Cl)ccc4O3)[C@@H](O)[C@H]2O)c(=O)[nH]c1=S ; [2H][C@@]1(n2cc(C)c(=S)[nH]c2=O)O[C@](F)(COP2(=O)OCc3cc(Cl)ccc3O2)[C@@H](O)[C@H]1O ; [2H]C([2H])(OP1(=O)OCc2cc(Cl)ccc2O1)[C@@]1(F)O[C@@H](n2cc(C)c(=S)[nH]c2=O)[C@H](O)[C@@H]1O ; [2H]C([2H])(OP1(=O)OCc2cc(Cl)ccc2O1)[C@@]1(F)O[C@@]([2H])(n2cc(C)c(=S)[nH]c2=O)[C@H](O)[C@@H]1O]

In exemplary embodiments, the compound is selected from:

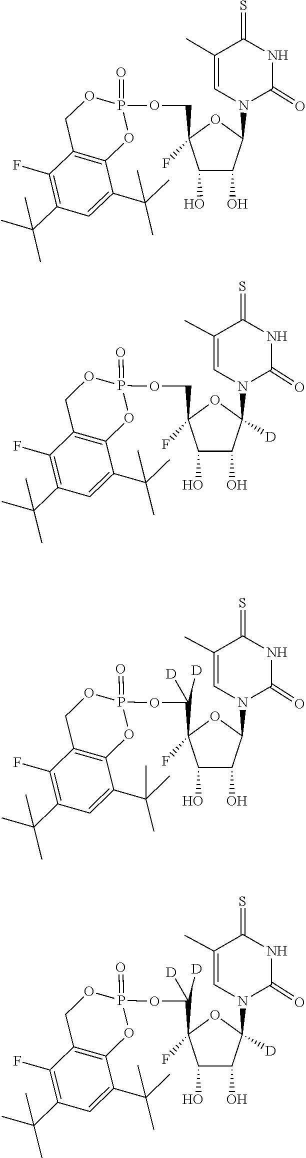 [SMILES: Cc1cn([C@@H]2O[C@](F)(COP3(=O)OCc4c(F)c(C(C)(C)C)cc(C(C)(C)C)c4O3)[C@@H](O)[C@H]2O)c(=O)[nH]c1=S ; [2H][C@@]1(n2cc(C)c(=S)[nH]c2=O)O[C@](F)(COP2(=O)OCc3c(F)c(C(C)(C)C)cc(C(C)(C)C)c3O2)[C@@H](O)[C@H]1O ; [2H]C([2H])(OP1(=O)OCc2c(F)c(C(C)(C)C)cc(C(C)(C)C)c2O1)[C@@]1(F)O[C@@H](n2cc(C)c(=S)[nH]c2=O)[C@H](O)[C@@H]1O ; [2H]C([2H])(OP1(=O)OCc2c(F)c(C(C)(C)C)cc(C(C)(C)C)c2O1)[C@@]1(F)O[C@@]([2H])(n2cc(C)c(=S)[nH]c2=O)[C@H](O)[C@@H]1O]

In exemplary embodiments, the compound is selected from:

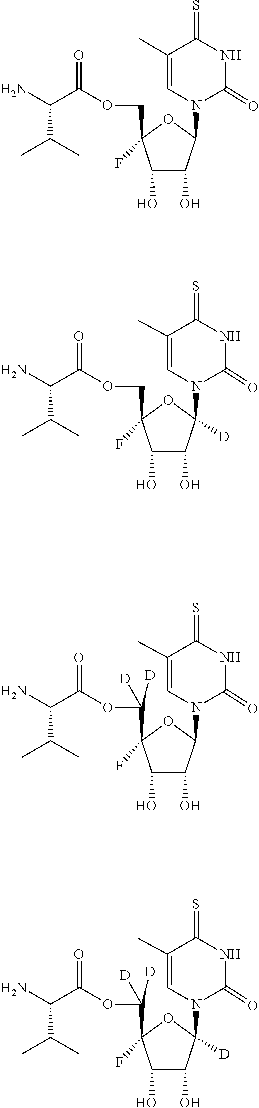 [SMILES: Cc1cn([C@@H]2O[C@](F)(COC(=O)[C@@H](N)C(C)C)[C@@H](O)[C@H]2O)c(=O)[nH]c1=S ; [2H][C@@]1(n2cc(C)c(=S)[nH]c2=O)O[C@](F)(COC(=O)[C@@H](N)C(C)C)[C@@H](O)[C@H]1O ; [2H]C([2H])(OC(=O)[C@@H](N)C(C)C)[C@@]1(F)O[C@@H](n2cc(C)c(=S)[nH]c2=O)[C@H](O)[C@@H]1O ; [2H]C([2H])(OC(=O)[C@@H](N)C(C)C)[C@@]1(F)O[C@@]([2H])(n2cc(C)c(=S)[nH]c2=O)[C@H](O)[C@@H]1O]

In exemplary embodiments, the compound is selected from:

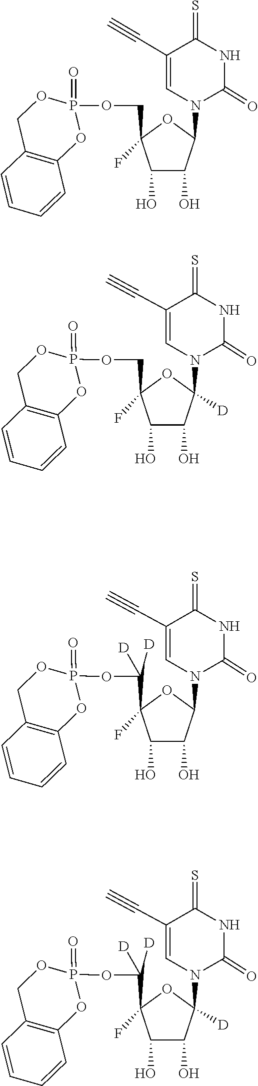 [SMILES: C#Cc1cn([C@@H]2O[C@](F)(COP3(=O)OCc4ccccc4O3)[C@@H](O)[C@H]2O)c(=O)[nH]c1=S ; [2H][C@@]1(n2cc(C#C)c(=S)[nH]c2=O)O[C@](F)(COP2(=O)OCc3ccccc3O2)[C@@H](O)[C@H]1O ; [2H]C([2H])(OP1(=O)OCc2ccccc2O1)[C@@]1(F)O[C@@H](n2cc(C#C)c(=S)[nH]c2=O)[C@H](O)[C@@H]1O ; [2H]C([2H])(OP1(=O)OCc2ccccc2O1)[C@@]1(F)O[C@@]([2H])(n2cc(C#C)c(=S)[nH]c2=O)[C@H](O)[C@@H]1O]

In exemplary embodiments, the compound is selected from:

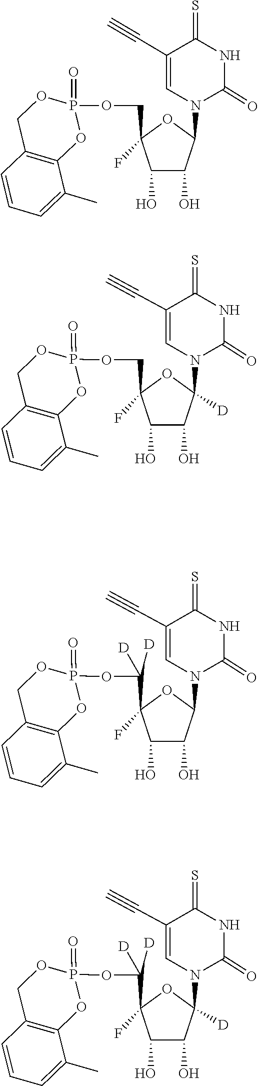 [SMILES: C#Cc1cn([C@@H]2O[C@](F)(COP3(=O)OCc4cccc(C)c4O3)[C@@H](O)[C@H]2O)c(=O)[nH]c1=S ; [2H][C@@]1(n2cc(C#C)c(=S)[nH]c2=O)O[C@](F)(COP2(=O)OCc3cccc(C)c3O2)[C@@H](O)[C@H]1O ; [2H]C([2H])(OP1(=O)OCc2cccc(C)c2O1)[C@@]1(F)O[C@@H](n2cc(C#C)c(=S)[nH]c2=O)[C@H](O)[C@@H]1O ; [2H]C([2H])(OP1(=O)OCc2cccc(C)c2O1)[C@@]1(F)O[C@@]([2H])(n2cc(C#C)c(=S)[nH]c2=O)[C@H](O)[C@@H]1O]

In exemplary embodiments, the compound is selected from:

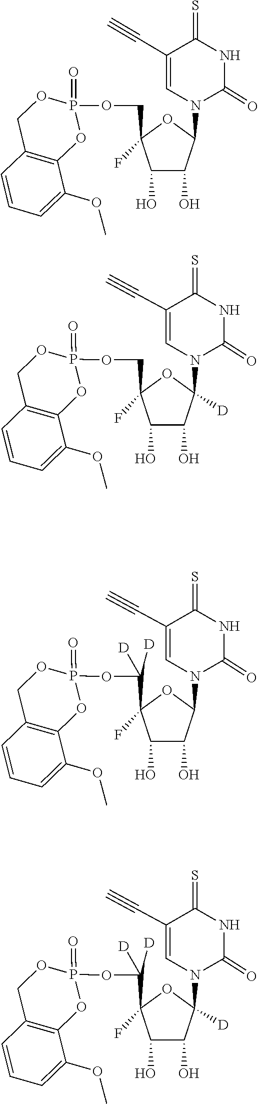

In exemplary embodiments, the compound is selected from:

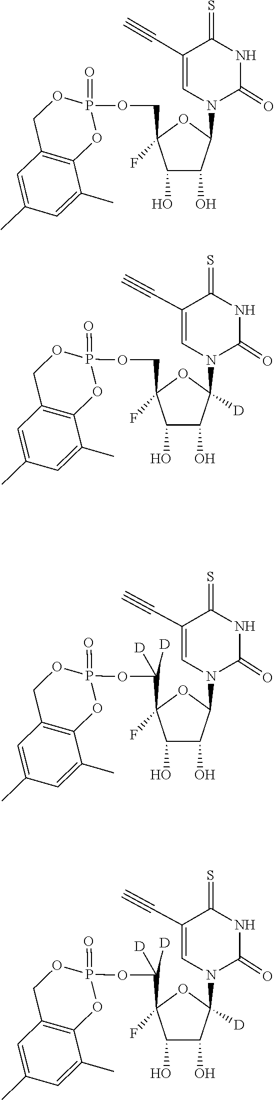 [SMILES: C#Cc1cn([C@@H]2O[C@](F)(COP3(=O)OCc4cc(C)cc(C)c4O3)[C@@H](O)[C@H]2O)c(=O)[nH]c1=S ; [2H][C@@]1(n2cc(C#C)c(=S)[nH]c2=O)O[C@](F)(COP2(=O)OCc3cc(C)cc(C)c3O2)[C@@H](O)[C@H]1O ; [2H]C([2H])(OP1(=O)OCc2cc(C)cc(C)c2O1)[C@@]1(F)O[C@@H](n2cc(C#C)c(=S)[nH]c2=O)[C@H](O)[C@@H]1O ; [2H]C([2H])(OP1(=O)OCc2cc(C)cc(C)c2O1)[C@@]1(F)O[C@@]([2H])(n2cc(C#C)c(=S)[nH]c2=O)[C@H](O)[C@@H]1O]

In exemplary embodiments, the compound is selected from:

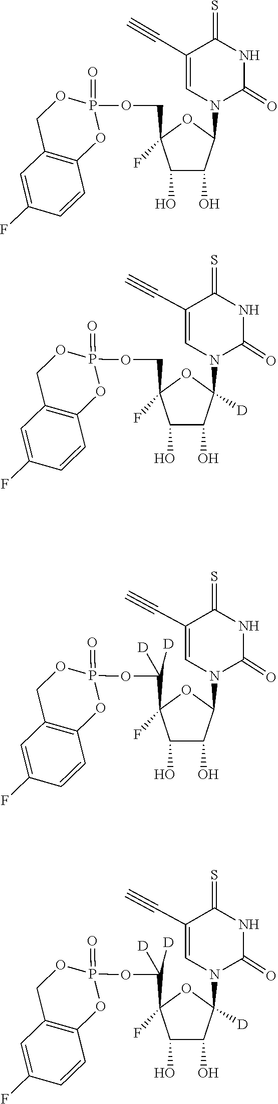 [SMILES: C#Cc1cn([C@@H]2O[C@](F)(COP3(=O)OCc4cc(F)ccc4O3)[C@@H](O)[C@H]2O)c(=O)[nH]c1=S ; [2H][C@@]1(n2cc(C#C)c(=S)[nH]c2=O)O[C@](F)(COP2(=O)OCc3cc(F)ccc3O2)[C@@H](O)[C@H]1O ; [2H]C([2H])(OP1(=O)OCc2cc(F)ccc2O1)[C@@]1(F)O[C@@H](n2cc(C#C)c(=S)[nH]c2=O)[C@H](O)[C@@H]1O ; [2H]C([2H])(OP1(=O)OCc2cc(F)ccc2O1)[C@@]1(F)O[C@@]([2H])(n2cc(C#C)c(=S)[nH]c2=O)[C@H](O)[C@@H]1O]

In exemplary embodiments, the compound is selected from:

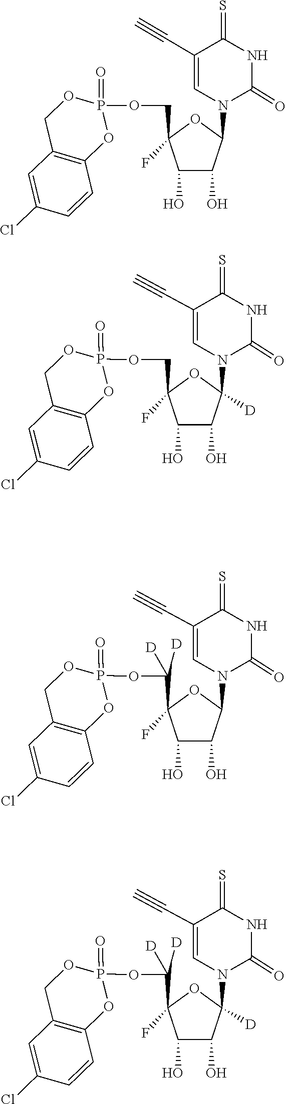 [SMILES: C#Cc1cn([C@@H]2O[C@](F)(COP3(=O)OCc4cc(Cl)ccc4O3)[C@@H](O)[C@H]2O)c(=O)[nH]c1=S ; [2H][C@@]1(n2cc(C#C)c(=S)[nH]c2=O)O[C@](F)(COP2(=O)OCc3cc(Cl)ccc3O2)[C@@H](O)[C@H]1O ; [2H]C([2H])(OP1(=O)OCc2cc(Cl)ccc2O1)[C@@]1(F)O[C@@H](n2cc(C#C)c(=S)[nH]c2=O)[C@H](O)[C@@H]1O ; [2H]C([2H])(OP1(=O)OCc2cc(Cl)ccc2O1)[C@@]1(F)O[C@@]([2H])(n2cc(C#C)c(=S)[nH]c2=O)[C@H](O)[C@@H]1O]

In exemplary embodiments, the compound is selected from:

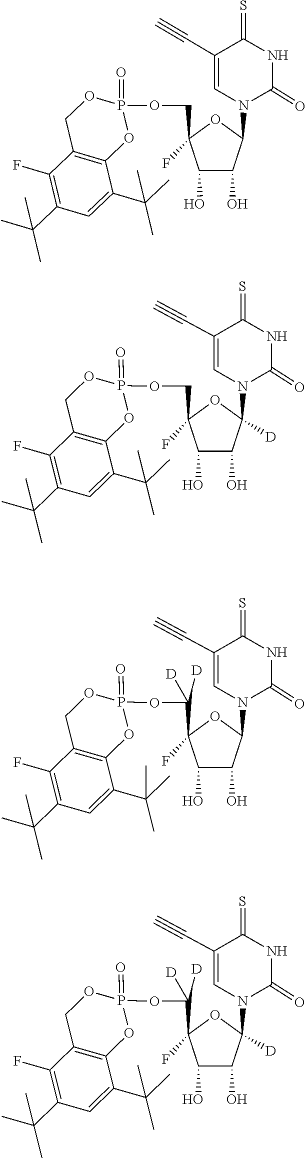 [SMILES: C#Cc1cn([C@@H]2O[C@](F)(COP3(=O)OCc4c(F)c(C(C)(C)C)cc(C(C)(C)C)c4O3)[C@@H](O)[C@H]2O)c(=O)[nH]c1=S ; [2H][C@@]1(n2cc(C#C)c(=S)[nH]c2=O)O[C@](F)(COP2(=O)OCc3c(F)c(C(C)(C)C)cc(C(C)(C)C)c3O2)[C@@H](O)[C@H]1O ; [2H]C([2H])(OP1(=O)OCc2c(F)c(C(C)(C)C)cc(C(C)(C)C)c2O1)[C@@]1(F)O[C@@H](n2cc(C#C)c(=S)[nH]c2=O)[C@H](O)[C@@H]1O ; [2H]C([2H])(OP1(=O)OCc2c(F)c(C(C)(C)C)cc(C(C)(C)C)c2O1)[C@@]1(F)O[C@@]([2H])(n2cc(C#C)c(=S)[nH]c2=O)[C@H](O)[C@@H]1O]

In exemplary embodiments, the compound is selected from:

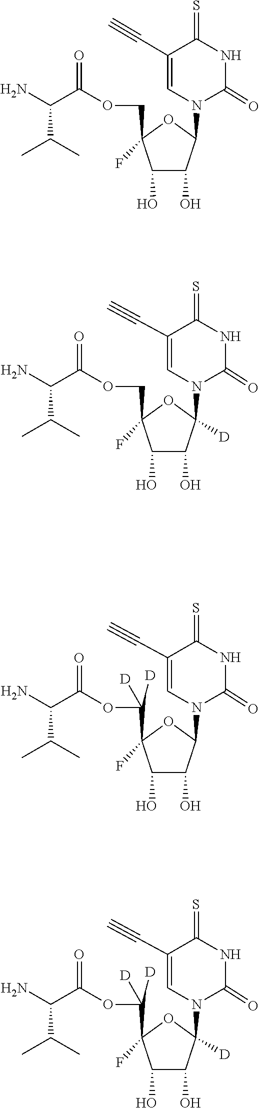 [SMILES: C#Cc1cn([C@@H]2O[C@](F)(COC(=O)[C@@H](N)C(C)C)[C@@H](O)[C@H]2O)c(=O)[nH]c1=S ; [2H][C@@]1(n2cc(C#C)c(=S)[nH]c2=O)O[C@](F)(COC(=O)[C@@H](N)C(C)C)[C@@H](O)[C@H]1O ; [2H]C([2H])(OC(=O)[C@@H](N)C(C)C)[C@@]1(F)O[C@@H](n2cc(C#C)c(=S)[nH]c2=O)[C@H](O)[C@@H]1O ; [2H]C([2H])(OC(=O)[C@@H](N)C(C)C)[C@@]1(F)O[C@@]([2H])(n2cc(C#C)c(=S)[nH]c2=O)[C@H](O)[C@@H]1O]

In exemplary embodiments, the compound is selected from:

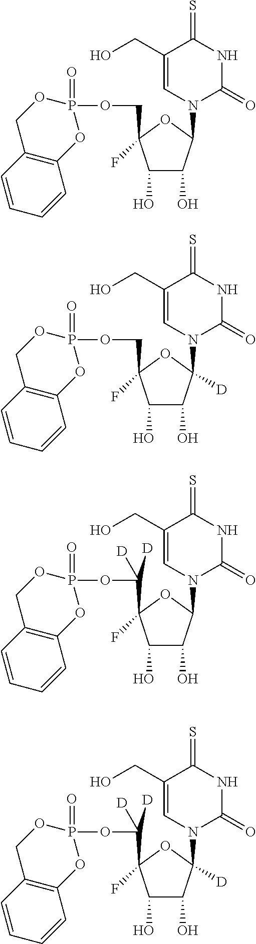

In exemplary embodiments, the compound is selected from:

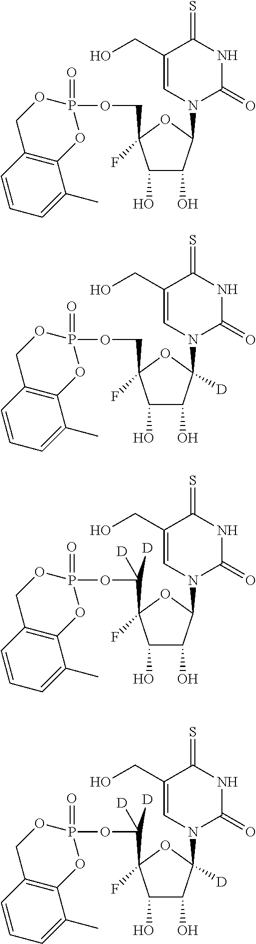

In exemplary embodiments, the compound is selected from:

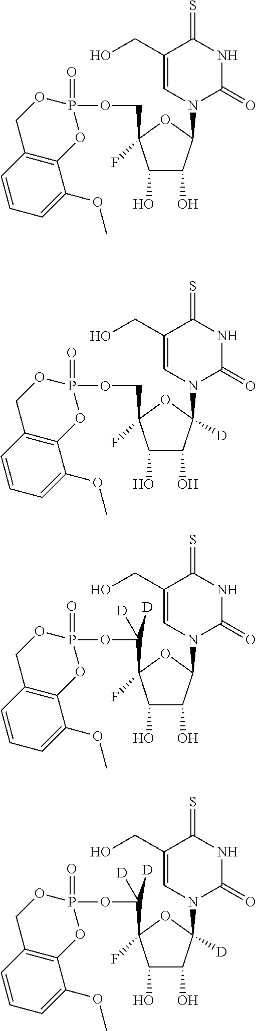

In exemplary embodiments, the compound is selected from:

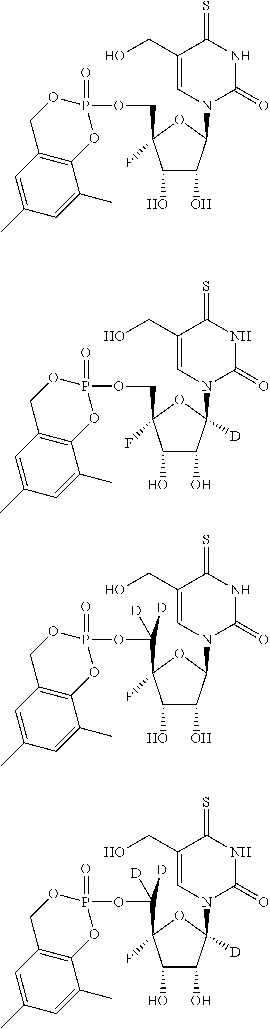

In exemplary embodiments, the compound is selected from:

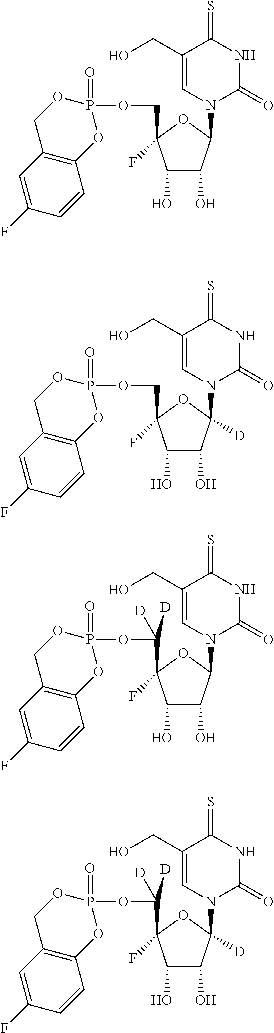

In exemplary embodiments, the compound is selected from:

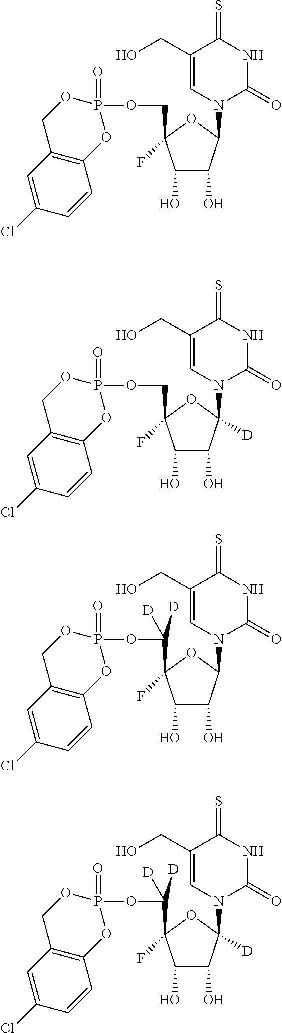

In exemplary embodiments, the compound is selected from:

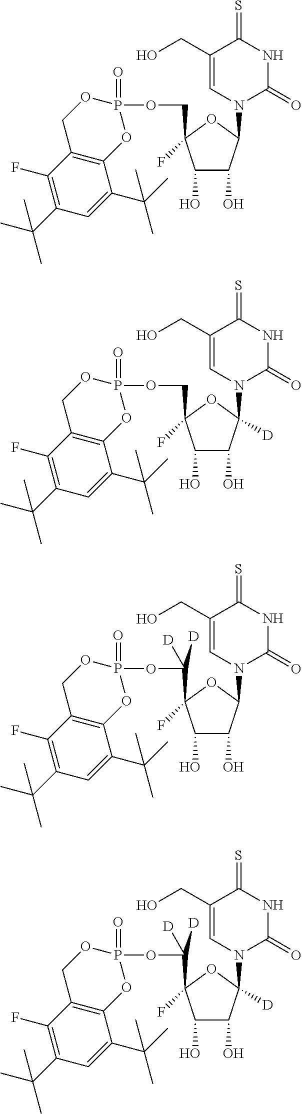

In exemplary embodiments, the compound is selected from:

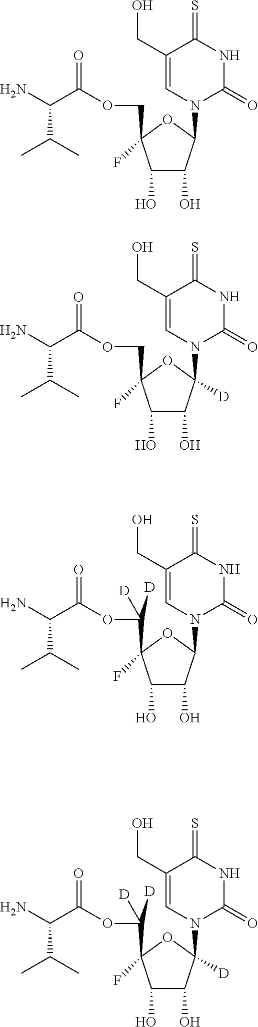

In exemplary embodiments, the compound is selected from:

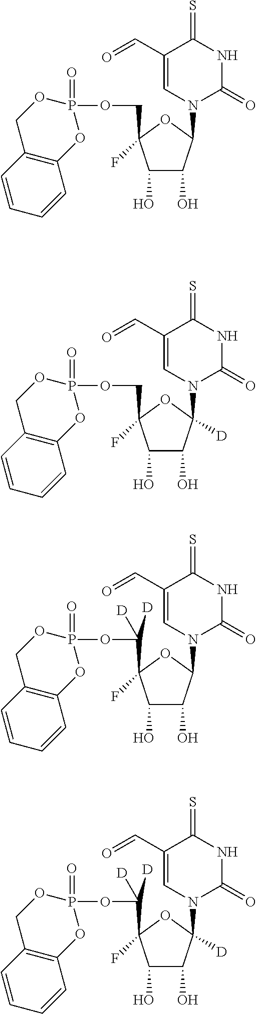

In exemplary embodiments, the compound is selected from:

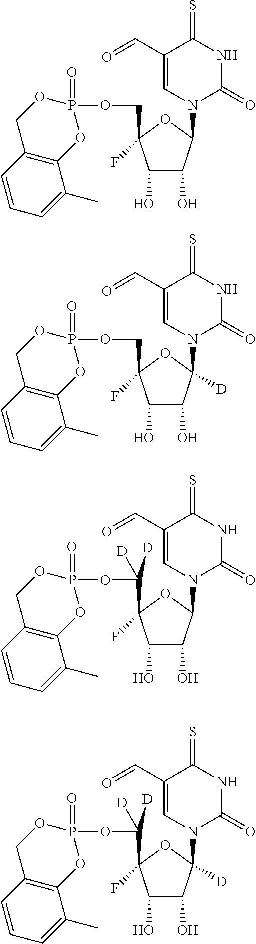

In exemplary embodiments, the compound is selected from:

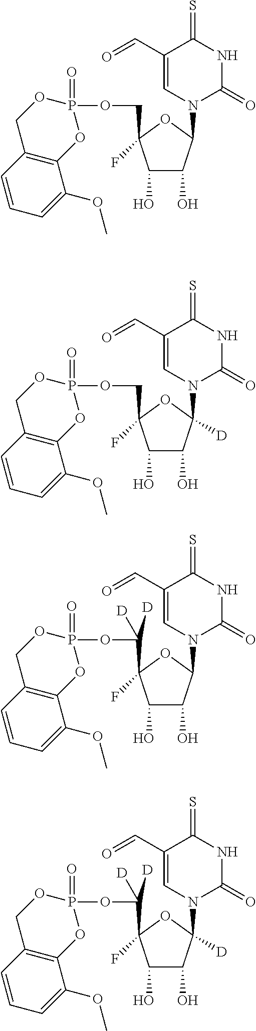

In exemplary embodiments, the compound is selected from:

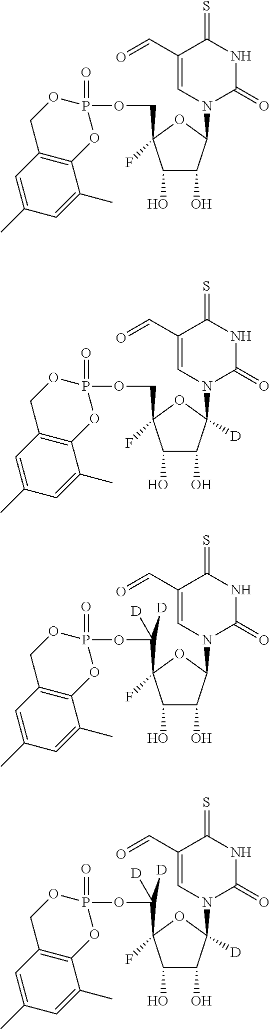

In exemplary embodiments, the compound is selected from:

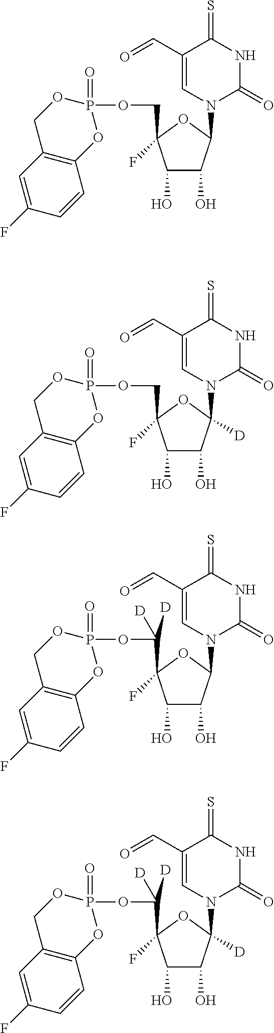

In exemplary embodiments, the compound is selected from:

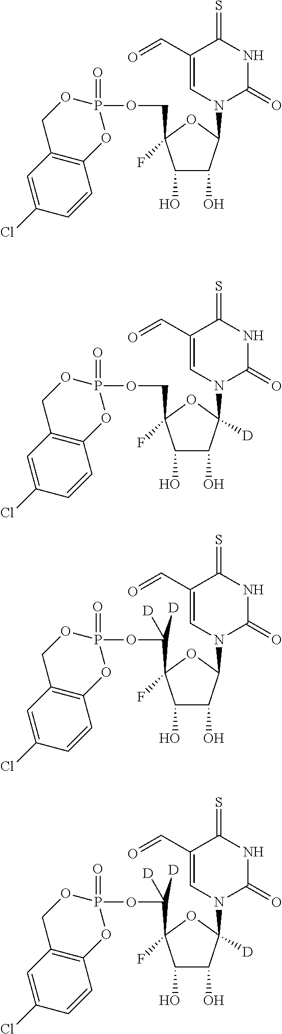

In exemplary embodiments, the compound is selected from:

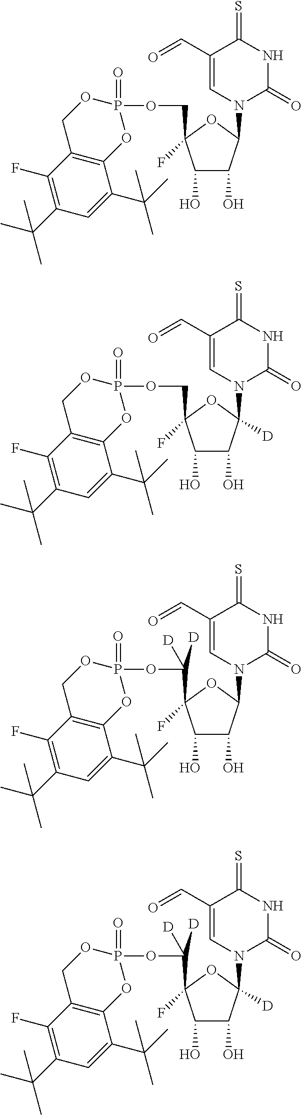

In exemplary embodiments, the compound is selected from:

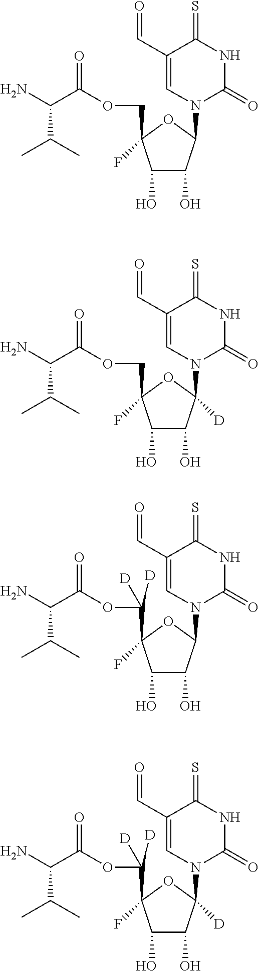

In exemplary embodiments, the compound is selected from:

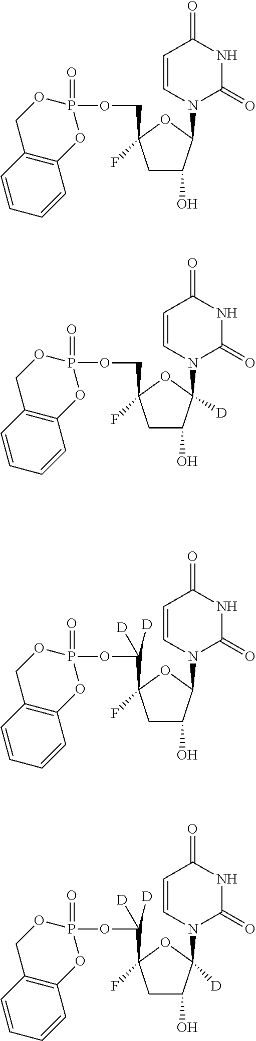 [SMILES: O=c1ccn([C@@H]2O[C@](F)(COP3(=O)OCc4ccccc4O3)C[C@H]2O)c(=O)[nH]1 ; [2H][C@@]1(n2ccc(=O)[nH]c2=O)O[C@](F)(COP2(=O)OCc3ccccc3O2)C[C@H]1O ; [2H]C([2H])(OP1(=O)OCc2ccccc2O1)[C@]1(F)C[C@@H](O)[C@H](n2ccc(=O)[nH]c2=O)O1 ; [2H]C([2H])(OP1(=O)OCc2ccccc2O1)[C@]1(F)C[C@@H](O)[C@]([2H])(n2ccc(=O)[nH]c2=O)O1]

In exemplary embodiments, the compound is selected from:

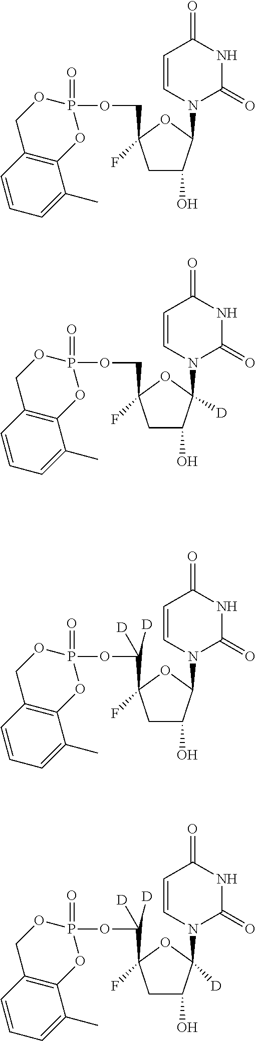 [SMILES: Cc1cccc2c1OP(=O)(OC[C@]1(F)C[C@@H](O)[C@H](n3ccc(=O)[nH]c3=O)O1)OC2 ; [2H][C@@]1(n2ccc(=O)[nH]c2=O)O[C@](F)(COP2(=O)OCc3cccc(C)c3O2)C[C@H]1O ; [2H]C([2H])(OP1(=O)OCc2cccc(C)c2O1)[C@]1(F)C[C@@H](O)[C@H](n2ccc(=O)[nH]c2=O)O1 ; [2H]C([2H])(OP1(=O)OCc2cccc(C)c2O1)[C@]1(F)C[C@@H](O)[C@]([2H])(n2ccc(=O)[nH]c2=O)O1]

In exemplary embodiments, the compound is selected from:

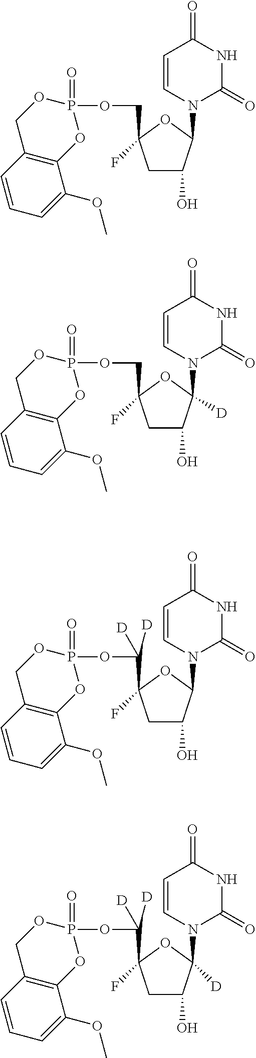 [SMILES: COc1cccc2c1OP(=O)(OC[C@]1(F)C[C@@H](O)[C@H](n3ccc(=O)[nH]c3=O)O1)OC2 ; [2H][C@@]1(n2ccc(=O)[nH]c2=O)O[C@](F)(COP2(=O)OCc3cccc(OC)c3O2)C[C@H]1O ; [2H]C([2H])(OP1(=O)OCc2cccc(OC)c2O1)[C@]1(F)C[C@@H](O)[C@H](n2ccc(=O)[nH]c2=O)O1 ; [2H]C([2H])(OP1(=O)OCc2cccc(OC)c2O1)[C@]1(F)C[C@@H](O)[C@]([2H])(n2ccc(=O)[nH]c2=O)O1]

In exemplary embodiments, the compound is selected from:

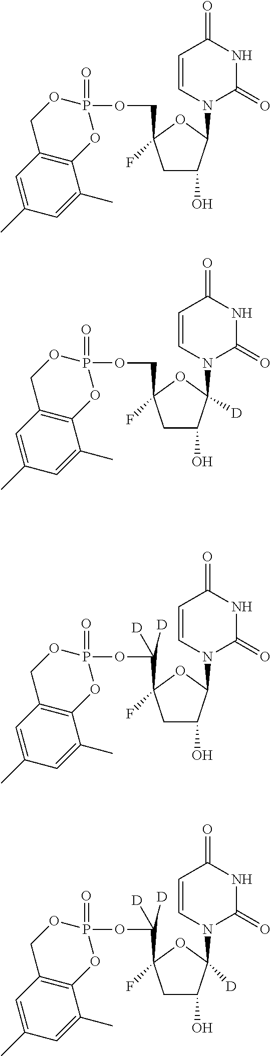 [SMILES: Cc1cc(C)c2c(c1)COP(=O)(OC[C@]1(F)C[C@@H](O)[C@H](n3ccc(=O)[nH]c3=O)O1)O2 ; [2H][C@@]1(n2ccc(=O)[nH]c2=O)O[C@](F)(COP2(=O)OCc3cc(C)cc(C)c3O2)C[C@H]1O ; [2H]C([2H])(OP1(=O)OCc2cc(C)cc(C)c2O1)[C@]1(F)C[C@@H](O)[C@H](n2ccc(=O)[nH]c2=O)O1 ; [2H]C([2H])(OP1(=O)OCc2cc(C)cc(C)c2O1)[C@]1(F)C[C@@H](O)[C@]([2H])(n2ccc(=O)[nH]c2=O)O1]

In exemplary embodiments, the compound is selected from:

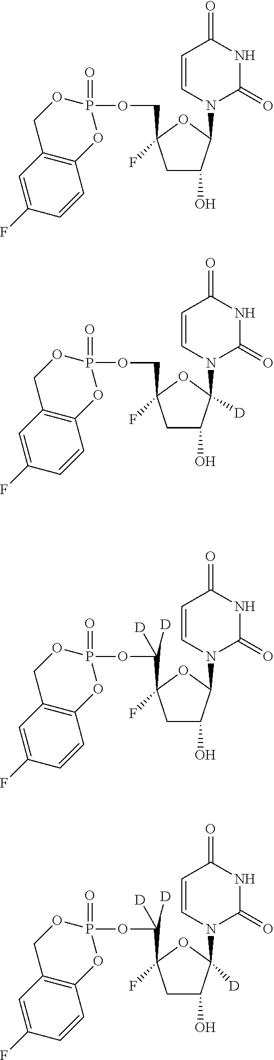 [SMILES: O=c1ccn([C@@H]2O[C@](F)(COP3(=O)OCc4cc(F)ccc4O3)C[C@H]2O)c(=O)[nH]1 ; [2H][C@@]1(n2ccc(=O)[nH]c2=O)O[C@](F)(COP2(=O)OCc3cc(F)ccc3O2)C[C@H]1O ; [2H]C([2H])(OP1(=O)OCc2cc(F)ccc2O1)[C@]1(F)C[C@@H](O)[C@H](n2ccc(=O)[nH]c2=O)O1 ; [2H]C([2H])(OP1(=O)OCc2cc(F)ccc2O1)[C@]1(F)C[C@@H](O)[C@]([2H])(n2ccc(=O)[nH]c2=O)O1]

In exemplary embodiments, the compound is selected from:

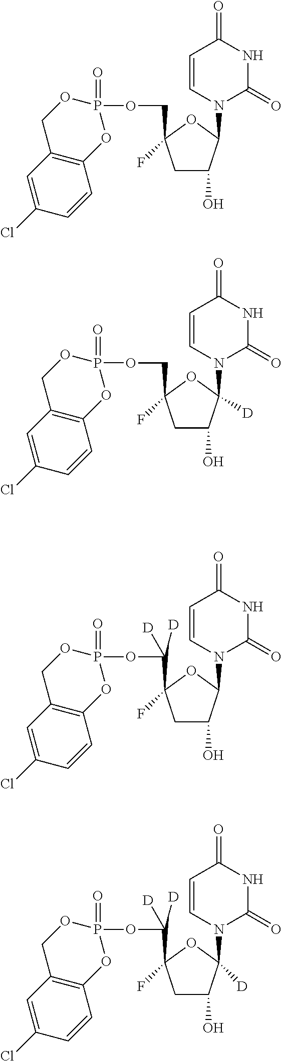 [SMILES: O=c1ccn([C@@H]2O[C@](F)(COP3(=O)OCc4cc(Cl)ccc4O3)C[C@H]2O)c(=O)[nH]1 ; [2H][C@@]1(n2ccc(=O)[nH]c2=O)O[C@](F)(COP2(=O)OCc3cc(Cl)ccc3O2)C[C@H]1O ; [2H]C([2H])(OP1(=O)OCc2cc(Cl)ccc2O1)[C@]1(F)C[C@@H](O)[C@H](n2ccc(=O)[nH]c2=O)O1 ; [2H]C([2H])(OP1(=O)OCc2cc(Cl)ccc2O1)[C@]1(F)C[C@@H](O)[C@]([2H])(n2ccc(=O)[nH]c2=O)O1]

In exemplary embodiments, the compound is selected from:

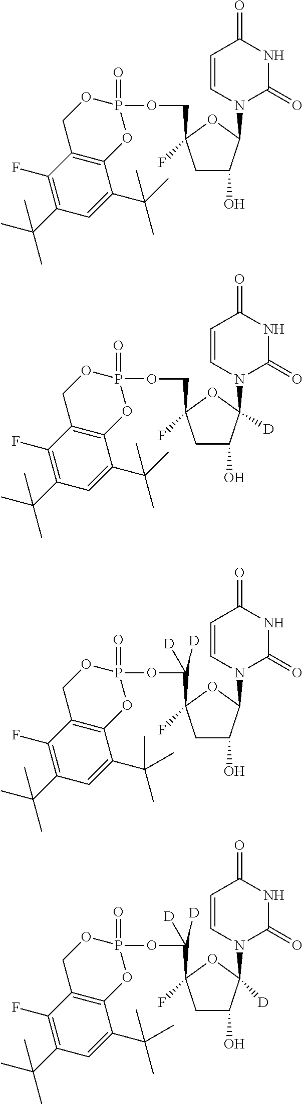 [SMILES: CC(C)(C)c1cc(C(C)(C)C)c2c(c1F)COP(=O)(OC[C@]1(F)C[C@@H](O)[C@H](n3ccc(=O)[nH]c3=O)O1)O2 ; [2H][C@@]1(n2ccc(=O)[nH]c2=O)O[C@](F)(COP2(=O)OCc3c(F)c(C(C)(C)C)cc(C(C)(C)C)c3O2)C[C@H]1O ; [2H]C([2H])(OP1(=O)OCc2c(F)c(C(C)(C)C)cc(C(C)(C)C)c2O1)[C@]1(F)C[C@@H](O)[C@H](n2ccc(=O)[nH]c2=O)O1 ; [2H]C([2H])(OP1(=O)OCc2c(F)c(C(C)(C)C)cc(C(C)(C)C)c2O1)[C@]1(F)C[C@@H](O)[C@]([2H])(n2ccc(=O)[nH]c2=O)O1]

In exemplary embodiments, the compound is selected from:

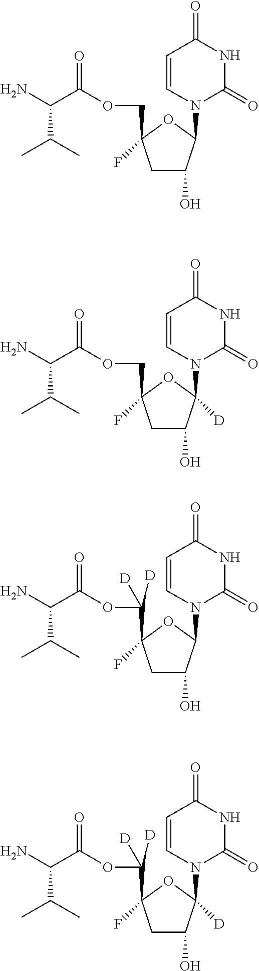

In exemplary embodiments, the compound is selected from:

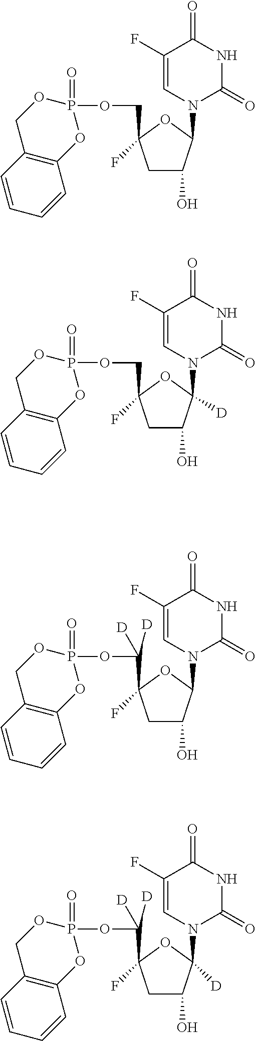 [SMILES: O=c1[nH]c(=O)n([C@@H]2O[C@](F)(COP3(=O)OCc4ccccc4O3)C[C@H]2O)cc1F ; [2H][C@@]1(n2cc(F)c(=O)[nH]c2=O)O[C@](F)(COP2(=O)OCc3ccccc3O2)C[C@H]1O ; [2H]C([2H])(OP1(=O)OCc2ccccc2O1)[C@]1(F)C[C@@H](O)[C@H](n2cc(F)c(=O)[nH]c2=O)O1 ; [2H]C([2H])(OP1(=O)OCc2ccccc2O1)[C@]1(F)C[C@@H](O)[C@]([2H])(n2cc(F)c(=O)[nH]c2=O)O1]

In exemplary embodiments, the compound is selected from:

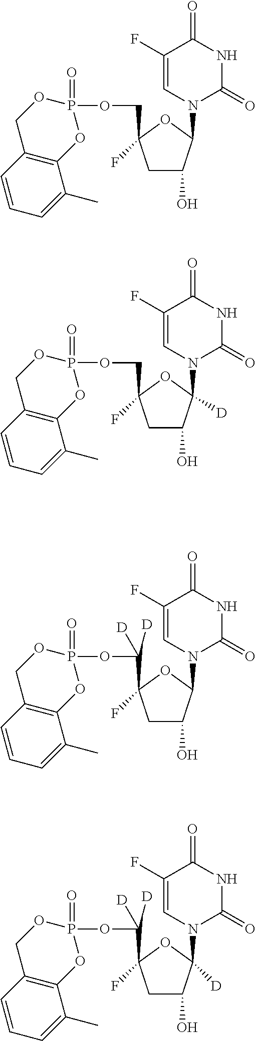 [SMILES: Cc1cccc2c1OP(=O)(OC[C@]1(F)C[C@@H](O)[C@H](n3cc(F)c(=O)[nH]c3=O)O1)OC2 ; [2H][C@@]1(n2cc(F)c(=O)[nH]c2=O)O[C@](F)(COP2(=O)OCc3cccc(C)c3O2)C[C@H]1O ; [2H]C([2H])(OP1(=O)OCc2cccc(C)c2O1)[C@]1(F)C[C@@H](O)[C@H](n2cc(F)c(=O)[nH]c2=O)O1 ; [2H]C([2H])(OP1(=O)OCc2cccc(C)c2O1)[C@]1(F)C[C@@H](O)[C@]([2H])(n2cc(F)c(=O)[nH]c2=O)O1]

In exemplary embodiments, the compound is selected from:

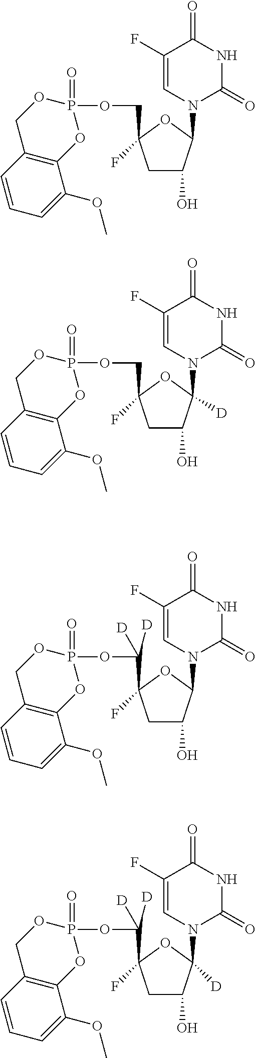 [SMILES: COc1cccc2c1OP(=O)(OC[C@]1(F)C[C@@H](O)[C@H](n3cc(F)c(=O)[nH]c3=O)O1)OC2 ; [2H][C@@]1(n2cc(F)c(=O)[nH]c2=O)O[C@](F)(COP2(=O)OCc3cccc(OC)c3O2)C[C@H]1O ; [2H]C([2H])(OP1(=O)OCc2cccc(OC)c2O1)[C@]1(F)C[C@@H](O)[C@H](n2cc(F)c(=O)[nH]c2=O)O1 ; [2H]C([2H])(OP1(=O)OCc2cccc(OC)c2O1)[C@]1(F)C[C@@H](O)[C@]([2H])(n2cc(F)c(=O)[nH]c2=O)O1]

In exemplary embodiments, the compound is selected from:

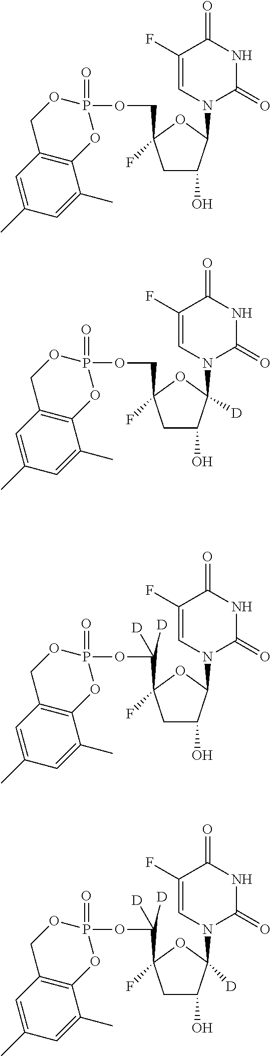 [SMILES: Cc1cc(C)c2c(c1)COP(=O)(OC[C@]1(F)C[C@@H](O)[C@H](n3cc(F)c(=O)[nH]c3=O)O1)O2 ; [2H][C@@]1(n2cc(F)c(=O)[nH]c2=O)O[C@](F)(COP2(=O)OCc3cc(C)cc(C)c3O2)C[C@H]1O ; [2H]C([2H])(OP1(=O)OCc2cc(C)cc(C)c2O1)[C@]1(F)C[C@@H](O)[C@H](n2cc(F)c(=O)[nH]c2=O)O1 ; [2H]C([2H])(OP1(=O)OCc2cc(C)cc(C)c2O1)[C@]1(F)C[C@@H](O)[C@]([2H])(n2cc(F)c(=O)[nH]c2=O)O1]

In exemplary embodiments, the compound is selected from:

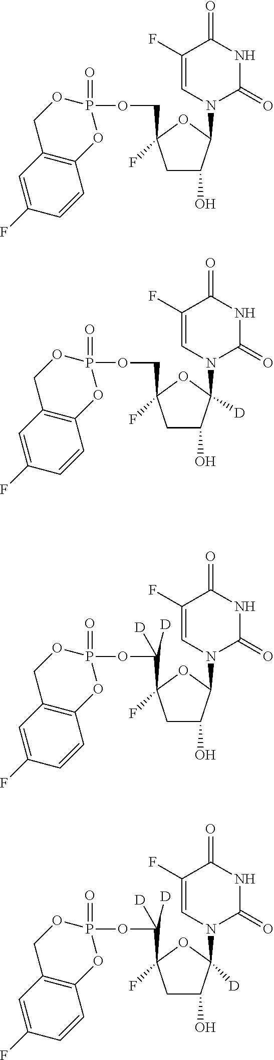 [SMILES: O=c1[nH]c(=O)n([C@@H]2O[C@](F)(COP3(=O)OCc4cc(F)ccc4O3)C[C@H]2O)cc1F ; [2H][C@@]1(n2cc(F)c(=O)[nH]c2=O)O[C@](F)(COP2(=O)OCc3cc(F)ccc3O2)C[C@H]1O ; [2H]C([2H])(OP1(=O)OCc2cc(F)ccc2O1)[C@]1(F)C[C@@H](O)[C@H](n2cc(F)c(=O)[nH]c2=O)O1 ; [2H]C([2H])(OP1(=O)OCc2cc(F)ccc2O1)[C@]1(F)C[C@@H](O)[C@]([2H])(n2cc(F)c(=O)[nH]c2=O)O1]

In exemplary embodiments, the compound is selected from:

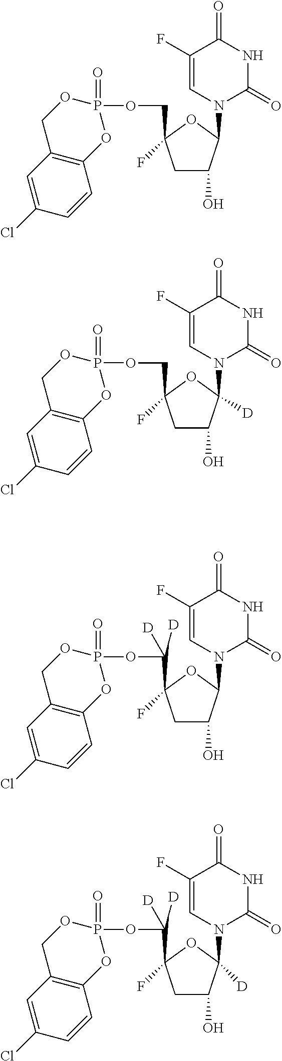 [SMILES: O=c1[nH]c(=O)n([C@@H]2O[C@](F)(COP3(=O)OCc4cc(Cl)ccc4O3)C[C@H]2O)cc1F ; [2H][C@@]1(n2cc(F)c(=O)[nH]c2=O)O[C@](F)(COP2(=O)OCc3cc(Cl)ccc3O2)C[C@H]1O ; [2H]C([2H])(OP1(=O)OCc2cc(Cl)ccc2O1)[C@]1(F)C[C@@H](O)[C@H](n2cc(F)c(=O)[nH]c2=O)O1 ; [2H]C([2H])(OP1(=O)OCc2cc(Cl)ccc2O1)[C@]1(F)C[C@@H](O)[C@]([2H])(n2cc(F)c(=O)[nH]c2=O)O1]

In exemplary embodiments, the compound is selected from:

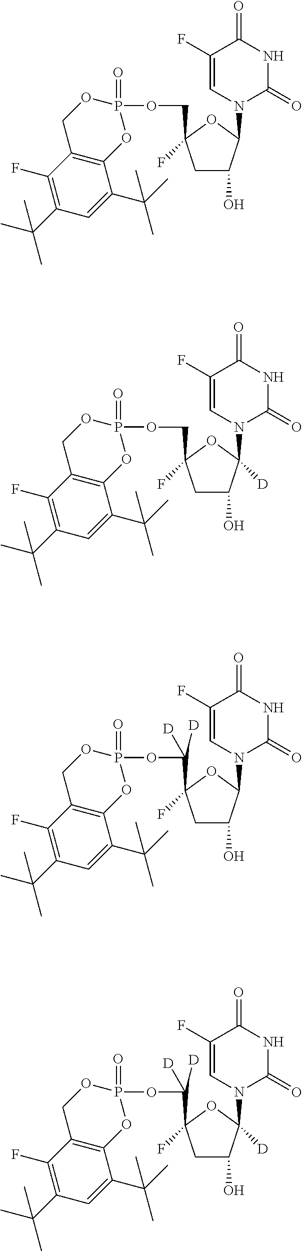 [SMILES: CC(C)(C)c1cc(C(C)(C)C)c2c(c1F)COP(=O)(OC[C@]1(F)C[C@@H](O)[C@H](n3cc(F)c(=O)[nH]c3=O)O1)O2 ; [2H][C@@]1(n2cc(F)c(=O)[nH]c2=O)O[C@](F)(COP2(=O)OCc3c(F)c(C(C)(C)C)cc(C(C)(C)C)c3O2)C[C@H]1O ; [2H]C([2H])(OP1(=O)OCc2c(F)c(C(C)(C)C)cc(C(C)(C)C)c2O1)[C@]1(F)C[C@@H](O)[C@H](n2cc(F)c(=O)[nH]c2=O)O1 ; [2H]C([2H])(OP1(=O)OCc2c(F)c(C(C)(C)C)cc(C(C)(C)C)c2O1)[C@]1(F)C[C@@H](O)[C@]([2H])(n2cc(F)c(=O)[nH]c2=O)O1]

In exemplary embodiments, the compound is selected from:

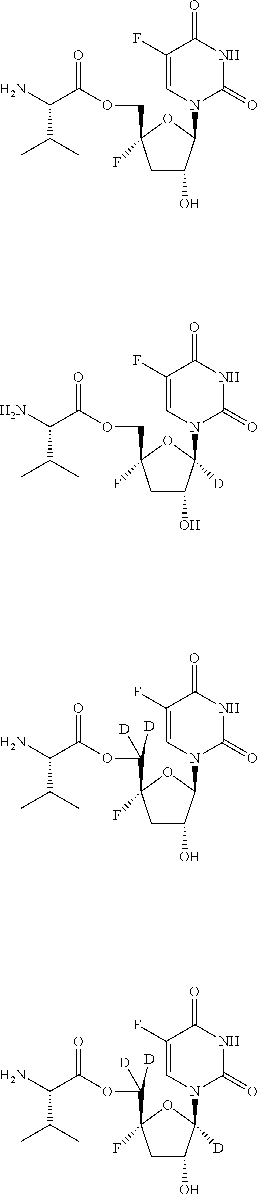 [SMILES: CC(C)[C@H](N)C(=O)OC[C@]1(F)C[C@@H](O)[C@H](n2cc(F)c(=O)[nH]c2=O)O1 ; [2H][C@@]1(n2cc(F)c(=O)[nH]c2=O)O[C@](F)(COC(=O)[C@@H](N)C(C)C)C[C@H]1O ; [2H]C([2H])(OC(=O)[C@@H](N)C(C)C)[C@]1(F)C[C@@H](O)[C@H](n2cc(F)c(=O)[nH]c2=O)O1 ; [2H]C([2H])(OC(=O)[C@@H](N)C(C)C)[C@]1(F)C[C@@H](O)[C@]([2H])(n2cc(F)c(=O)[nH]c2=O)O1]

In exemplary embodiments, the compound is selected from:

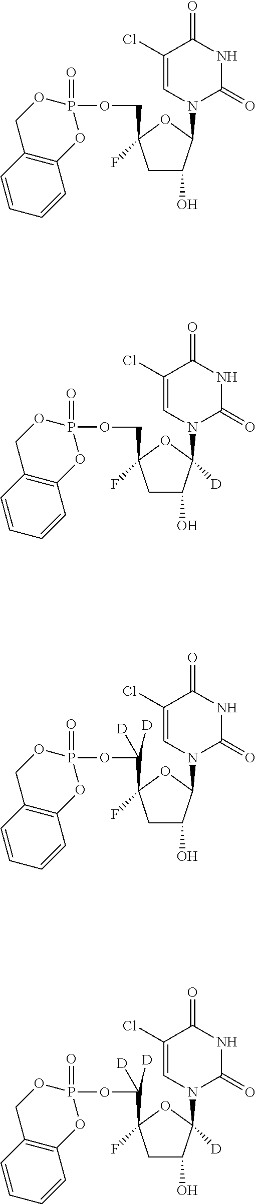 [SMILES: O=c1[nH]c(=O)n([C@@H]2O[C@](F)(COP3(=O)OCc4ccccc4O3)C[C@H]2O)cc1Cl ; [2H][C@@]1(n2cc(Cl)c(=O)[nH]c2=O)O[C@](F)(COP2(=O)OCc3ccccc3O2)C[C@H]1O ; [2H]C([2H])(OP1(=O)OCc2ccccc2O1)[C@]1(F)C[C@@H](O)[C@H](n2cc(Cl)c(=O)[nH]c2=O)O1 ; [2H]C([2H])(OP1(=O)OCc2ccccc2O1)[C@]1(F)C[C@@H](O)[C@]([2H])(n2cc(Cl)c(=O)[nH]c2=O)O1]

In exemplary embodiments, the compound is selected from:

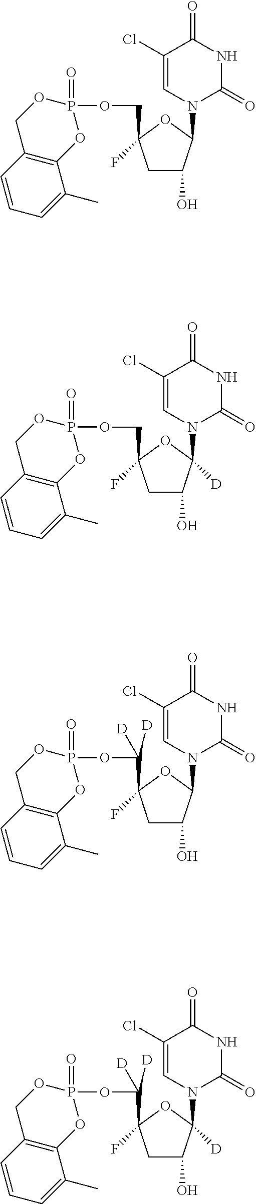 [SMILES: Cc1cccc2c1OP(=O)(OC[C@]1(F)C[C@@H](O)[C@H](n3cc(Cl)c(=O)[nH]c3=O)O1)OC2 ; [2H][C@@]1(n2cc(Cl)c(=O)[nH]c2=O)O[C@](F)(COP2(=O)OCc3cccc(C)c3O2)C[C@H]1O ; [2H]C([2H])(OP1(=O)OCc2cccc(C)c2O1)[C@]1(F)C[C@@H](O)[C@H](n2cc(Cl)c(=O)[nH]c2=O)O1 ; [2H]C([2H])(OP1(=O)OCc2cccc(C)c2O1)[C@]1(F)C[C@@H](O)[C@]([2H])(n2cc(Cl)c(=O)[nH]c2=O)O1]

In exemplary embodiments, the compound is selected from:

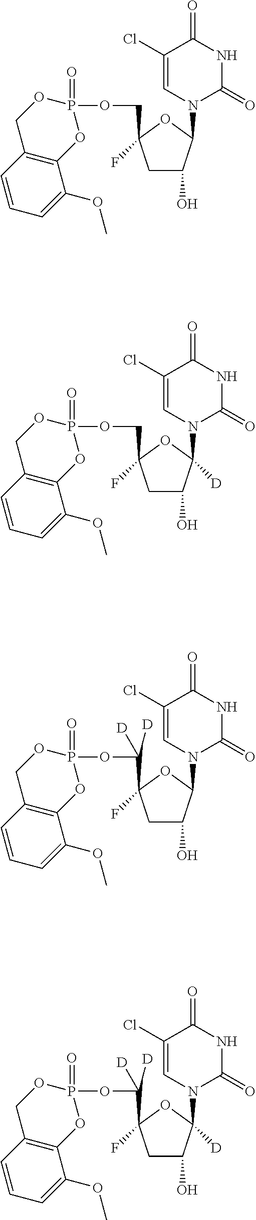 [SMILES: COc1cccc2c1OP(=O)(OC[C@]1(F)C[C@@H](O)[C@H](n3cc(Cl)c(=O)[nH]c3=O)O1)OC2 ; [2H][C@@]1(n2cc(Cl)c(=O)[nH]c2=O)O[C@](F)(COP2(=O)OCc3cccc(OC)c3O2)C[C@H]1O ; [2H]C([2H])(OP1(=O)OCc2cccc(OC)c2O1)[C@]1(F)C[C@@H](O)[C@H](n2cc(Cl)c(=O)[nH]c2=O)O1 ; [2H]C([2H])(OP1(=O)OCc2cccc(OC)c2O1)[C@]1(F)C[C@@H](O)[C@]([2H])(n2cc(Cl)c(=O)[nH]c2=O)O1]

In exemplary embodiments, the compound is selected from:

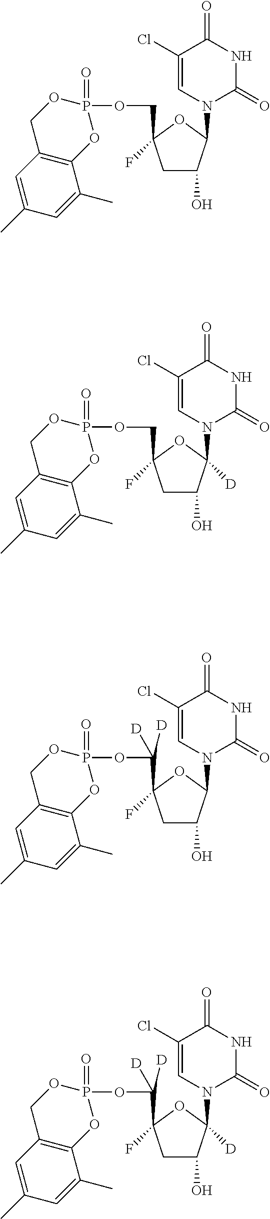 [SMILES: Cc1cc(C)c2c(c1)COP(=O)(OC[C@]1(F)C[C@@H](O)[C@H](n3cc(Cl)c(=O)[nH]c3=O)O1)O2 ; [2H][C@@]1(n2cc(Cl)c(=O)[nH]c2=O)O[C@](F)(COP2(=O)OCc3cc(C)cc(C)c3O2)C[C@H]1O ; [2H]C([2H])(OP1(=O)OCc2cc(C)cc(C)c2O1)[C@]1(F)C[C@@H](O)[C@H](n2cc(Cl)c(=O)[nH]c2=O)O1 ; [2H]C([2H])(OP1(=O)OCc2cc(C)cc(C)c2O1)[C@]1(F)C[C@@H](O)[C@]([2H])(n2cc(Cl)c(=O)[nH]c2=O)O1]

In exemplary embodiments, the compound is selected from:

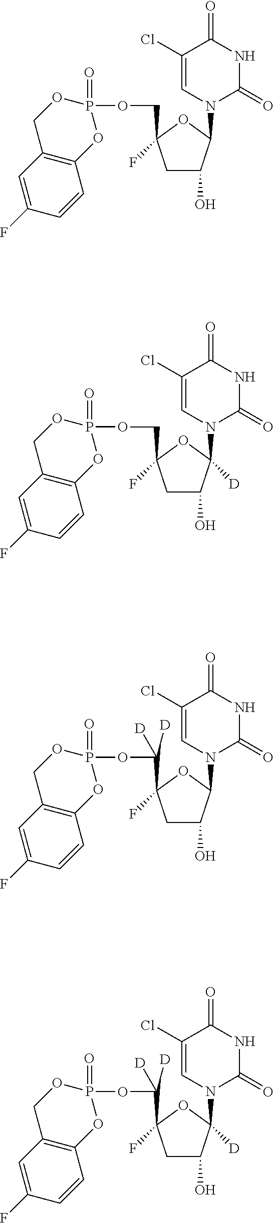 [SMILES: O=c1[nH]c(=O)n([C@@H]2O[C@](F)(COP3(=O)OCc4cc(F)ccc4O3)C[C@H]2O)cc1Cl ; [2H][C@@]1(n2cc(Cl)c(=O)[nH]c2=O)O[C@](F)(COP2(=O)OCc3cc(F)ccc3O2)C[C@H]1O ; [2H]C([2H])(OP1(=O)OCc2cc(F)ccc2O1)[C@]1(F)C[C@@H](O)[C@H](n2cc(Cl)c(=O)[nH]c2=O)O1 ; [2H]C([2H])(OP1(=O)OCc2cc(F)ccc2O1)[C@]1(F)C[C@@H](O)[C@]([2H])(n2cc(Cl)c(=O)[nH]c2=O)O1]

In exemplary embodiments, the compound is selected from:

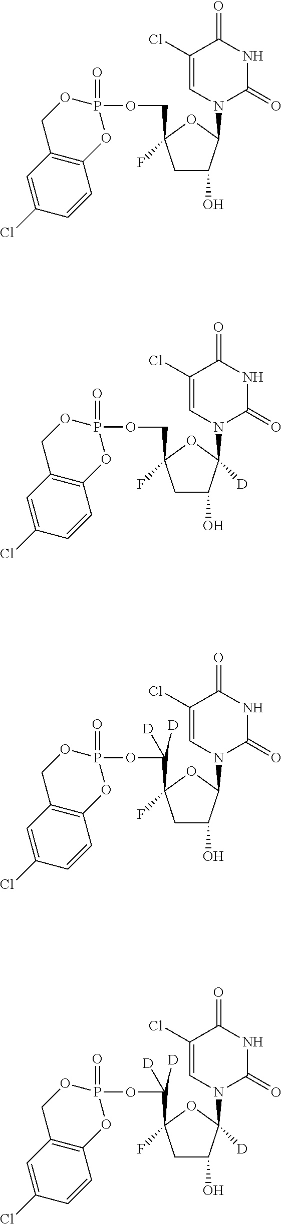 [SMILES: O=c1[nH]c(=O)n([C@@H]2O[C@](F)(COP3(=O)OCc4cc(Cl)ccc4O3)C[C@H]2O)cc1Cl ; [2H][C@@]1(n2cc(Cl)c(=O)[nH]c2=O)O[C@](F)(COP2(=O)OCc3cc(Cl)ccc3O2)C[C@H]1O ; [2H]C([2H])(OP1(=O)OCc2cc(Cl)ccc2O1)[C@]1(F)C[C@@H](O)[C@H](n2cc(Cl)c(=O)[nH]c2=O)O1 ; [2H]C([2H])(OP1(=O)OCc2cc(Cl)ccc2O1)[C@]1(F)C[C@@H](O)[C@]([2H])(n2cc(Cl)c(=O)[nH]c2=O)O1]

In exemplary embodiments, the compound is selected from:

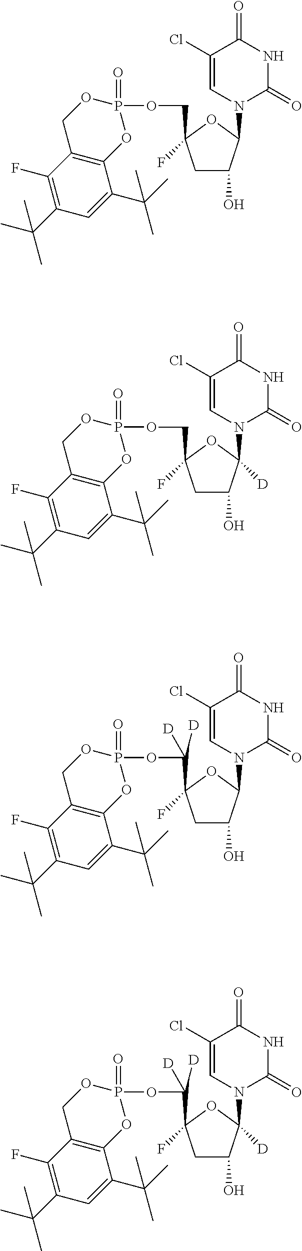 [SMILES: CC(C)(C)c1cc(C(C)(C)C)c2c(c1F)COP(=O)(OC[C@]1(F)C[C@@H](O)[C@H](n3cc(Cl)c(=O)[nH]c3=O)O1)O2 ; [2H][C@@]1(n2cc(Cl)c(=O)[nH]c2=O)O[C@](F)(COP2(=O)OCc3c(F)c(C(C)(C)C)cc(C(C)(C)C)c3O2)C[C@H]1O ; [2H]C([2H])(OP1(=O)OCc2c(F)c(C(C)(C)C)cc(C(C)(C)C)c2O1)[C@]1(F)C[C@@H](O)[C@H](n2cc(Cl)c(=O)[nH]c2=O)O1 ; [2H]C([2H])(OP1(=O)OCc2c(F)c(C(C)(C)C)cc(C(C)(C)C)c2O1)[C@]1(F)C[C@@H](O)[C@]([2H])(n2cc(Cl)c(=O)[nH]c2=O)O1]

In exemplary embodiments, the compound is selected from:

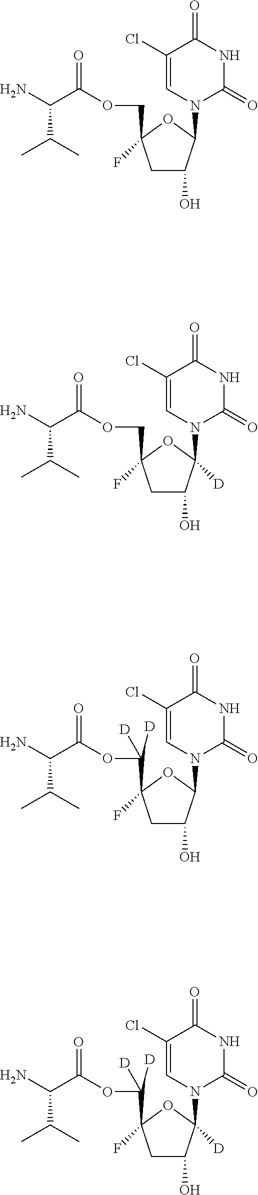 [SMILES: CC(C)[C@H](N)C(=O)OC[C@]1(F)C[C@@H](O)[C@H](n2cc(Cl)c(=O)[nH]c2=O)O1 ; [2H][C@@]1(n2cc(Cl)c(=O)[nH]c2=O)O[C@](F)(COC(=O)[C@@H](N)C(C)C)C[C@H]1O ; [2H]C([2H])(OC(=O)[C@@H](N)C(C)C)[C@]1(F)C[C@@H](O)[C@H](n2cc(Cl)c(=O)[nH]c2=O)O1 ; [2H]C([2H])(OC(=O)[C@@H](N)C(C)C)[C@]1(F)C[C@@H](O)[C@]([2H])(n2cc(Cl)c(=O)[nH]c2=O)O1]

In exemplary embodiments, the compound is selected from:

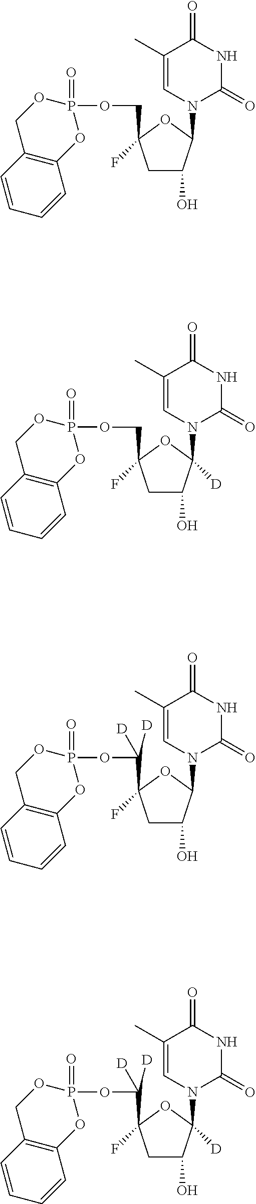 [SMILES: Cc1cn([C@@H]2O[C@](F)(COP3(=O)OCc4ccccc4O3)C[C@H]2O)c(=O)[nH]c1=O ; [2H][C@@]1(n2cc(C)c(=O)[nH]c2=O)O[C@](F)(COP2(=O)OCc3ccccc3O2)C[C@H]1O ; [2H]C([2H])(OP1(=O)OCc2ccccc2O1)[C@]1(F)C[C@@H](O)[C@H](n2cc(C)c(=O)[nH]c2=O)O1 ; [2H]C([2H])(OP1(=O)OCc2ccccc2O1)[C@]1(F)C[C@@H](O)[C@]([2H])(n2cc(C)c(=O)[nH]c2=O)O1]

In exemplary embodiments, the compound is selected from:

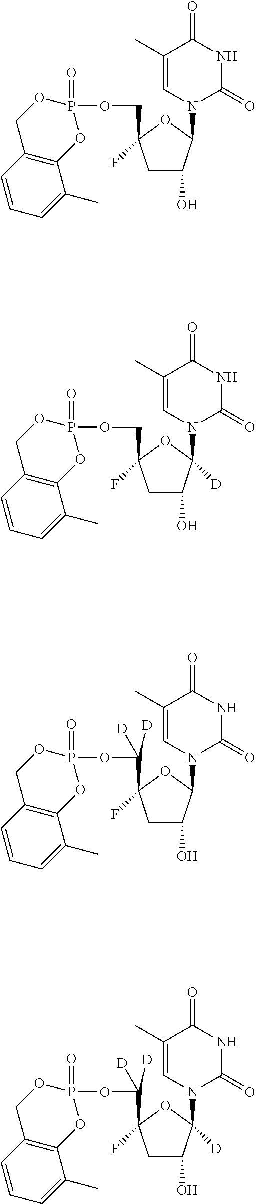 [SMILES: Cc1cccc2c1OP(=O)(OC[C@]1(F)C[C@@H](O)[C@H](n3cc(C)c(=O)[nH]c3=O)O1)OC2 ; [2H][C@@]1(n2cc(C)c(=O)[nH]c2=O)O[C@](F)(COP2(=O)OCc3cccc(C)c3O2)C[C@H]1O ; [2H]C([2H])(OP1(=O)OCc2cccc(C)c2O1)[C@]1(F)C[C@@H](O)[C@H](n2cc(C)c(=O)[nH]c2=O)O1 ; [2H]C([2H])(OP1(=O)OCc2cccc(C)c2O1)[C@]1(F)C[C@@H](O)[C@]([2H])(n2cc(C)c(=O)[nH]c2=O)O1]

In exemplary embodiments, the compound is selected from:

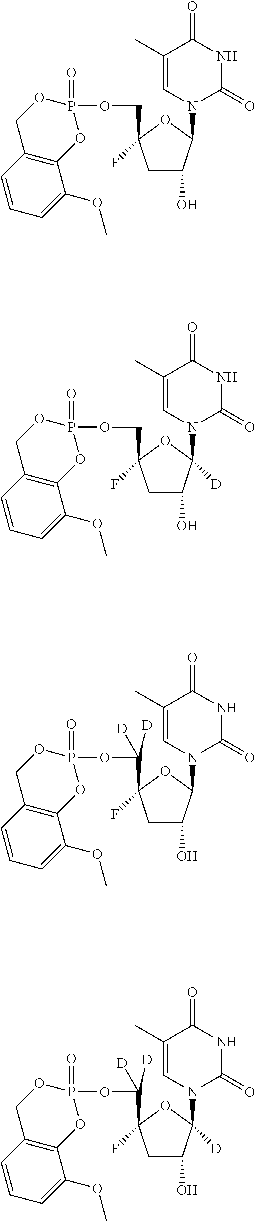 [SMILES: COc1cccc2c1OP(=O)(OC[C@]1(F)C[C@@H](O)[C@H](n3cc(C)c(=O)[nH]c3=O)O1)OC2 ; [2H][C@@]1(n2cc(C)c(=O)[nH]c2=O)O[C@](F)(COP2(=O)OCc3cccc(OC)c3O2)C[C@H]1O ; [2H]C([2H])(OP1(=O)OCc2cccc(OC)c2O1)[C@]1(F)C[C@@H](O)[C@H](n2cc(C)c(=O)[nH]c2=O)O1 ; [2H]C([2H])(OP1(=O)OCc2cccc(OC)c2O1)[C@]1(F)C[C@@H](O)[C@]([2H])(n2cc(C)c(=O)[nH]c2=O)O1]

In exemplary embodiments, the compound is selected from:

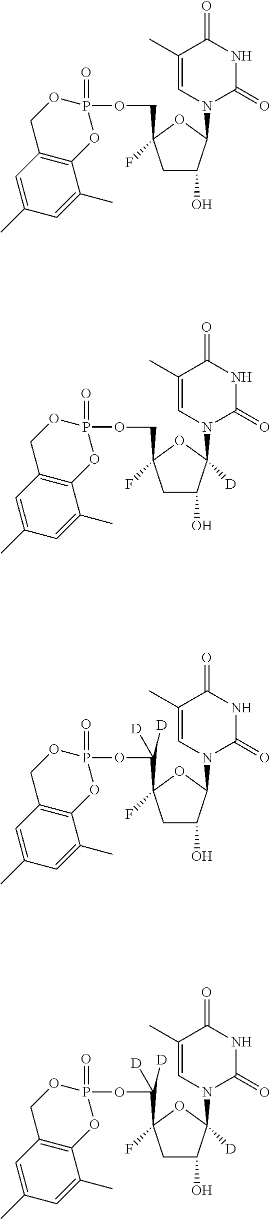 [SMILES: Cc1cc(C)c2c(c1)COP(=O)(OC[C@]1(F)C[C@@H](O)[C@H](n3cc(C)c(=O)[nH]c3=O)O1)O2 ; [2H][C@@]1(n2cc(C)c(=O)[nH]c2=O)O[C@](F)(COP2(=O)OCc3cc(C)cc(C)c3O2)C[C@H]1O ; [2H]C([2H])(OP1(=O)OCc2cc(C)cc(C)c2O1)[C@]1(F)C[C@@H](O)[C@H](n2cc(C)c(=O)[nH]c2=O)O1 ; [2H]C([2H])(OP1(=O)OCc2cc(C)cc(C)c2O1)[C@]1(F)C[C@@H](O)[C@]([2H])(n2cc(C)c(=O)[nH]c2=O)O1]

In exemplary embodiments, the compound is selected from:

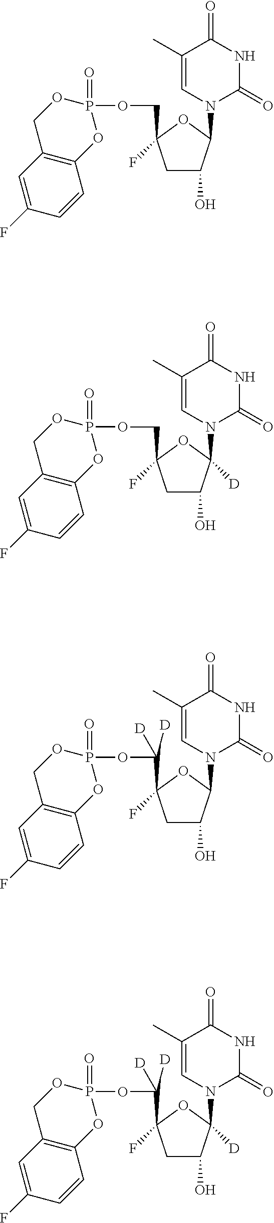 [SMILES: Cc1cn([C@@H]2O[C@](F)(COP3(=O)OCc4cc(F)ccc4O3)C[C@H]2O)c(=O)[nH]c1=O ; [2H][C@@]1(n2cc(C)c(=O)[nH]c2=O)O[C@](F)(COP2(=O)OCc3cc(F)ccc3O2)C[C@H]1O ; [2H]C([2H])(OP1(=O)OCc2cc(F)ccc2O1)[C@]1(F)C[C@@H](O)[C@H](n2cc(C)c(=O)[nH]c2=O)O1 ; [2H]C([2H])(OP1(=O)OCc2cc(F)ccc2O1)[C@]1(F)C[C@@H](O)[C@]([2H])(n2cc(C)c(=O)[nH]c2=O)O1]

In exemplary embodiments, the compound is selected from:

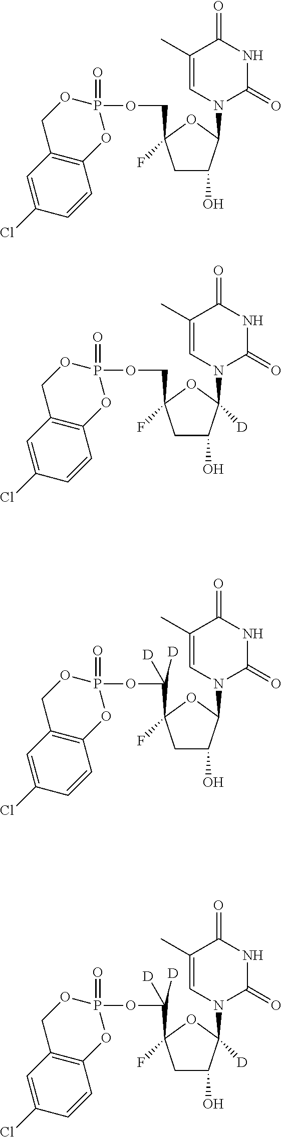 [SMILES: Cc1cn([C@@H]2O[C@](F)(COP3(=O)OCc4cc(Cl)ccc4O3)C[C@H]2O)c(=O)[nH]c1=O ; [2H][C@@]1(n2cc(C)c(=O)[nH]c2=O)O[C@](F)(COP2(=O)OCc3cc(Cl)ccc3O2)C[C@H]1O ; [2H]C([2H])(OP1(=O)OCc2cc(Cl)ccc2O1)[C@]1(F)C[C@@H](O)[C@H](n2cc(C)c(=O)[nH]c2=O)O1 ; [2H]C([2H])(OP1(=O)OCc2cc(Cl)ccc2O1)[C@]1(F)C[C@@H](O)[C@]([2H])(n2cc(C)c(=O)[nH]c2=O)O1]

In exemplary embodiments, the compound is selected from:

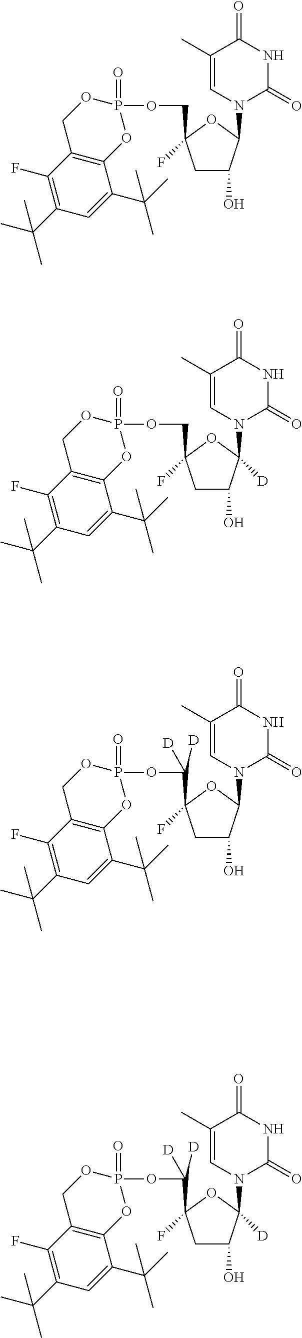 [SMILES: Cc1cn([C@@H]2O[C@](F)(COP3(=O)OCc4c(F)c(C(C)(C)C)cc(C(C)(C)C)c4O3)C[C@H]2O)c(=O)[nH]c1=O ; [2H][C@@]1(n2cc(C)c(=O)[nH]c2=O)O[C@](F)(COP2(=O)OCc3c(F)c(C(C)(C)C)cc(C(C)(C)C)c3O2)C[C@H]1O ; [2H]C([2H])(OP1(=O)OCc2c(F)c(C(C)(C)C)cc(C(C)(C)C)c2O1)[C@]1(F)C[C@@H](O)[C@H](n2cc(C)c(=O)[nH]c2=O)O1 ; [2H]C([2H])(OP1(=O)OCc2c(F)c(C(C)(C)C)cc(C(C)(C)C)c2O1)[C@]1(F)C[C@@H](O)[C@]([2H])(n2cc(C)c(=O)[nH]c2=O)O1]

In exemplary embodiments, the compound is selected from:

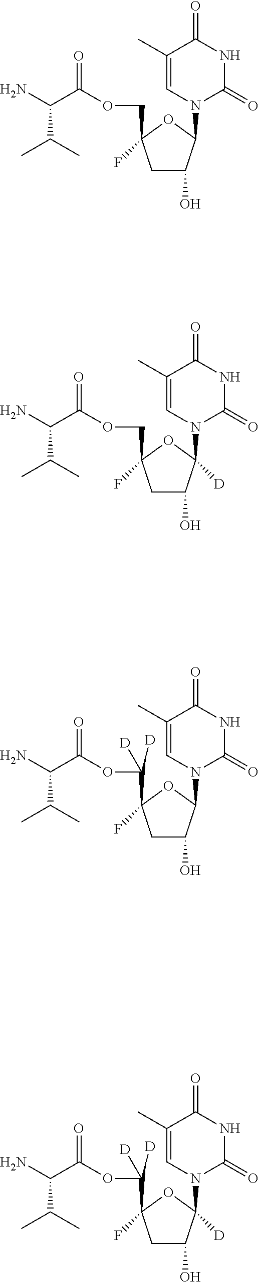 [SMILES: Cc1cn([C@@H]2O[C@](F)(COC(=O)[C@@H](N)C(C)C)C[C@H]2O)c(=O)[nH]c1=O ; [2H][C@@]1(n2cc(C)c(=O)[nH]c2=O)O[C@](F)(COC(=O)[C@@H](N)C(C)C)C[C@H]1O ; [2H]C([2H])(OC(=O)[C@@H](N)C(C)C)[C@]1(F)C[C@@H](O)[C@H](n2cc(C)c(=O)[nH]c2=O)O1 ; [2H]C([2H])(OC(=O)[C@@H](N)C(C)C)[C@]1(F)C[C@@H](O)[C@]([2H])(n2cc(C)c(=O)[nH]c2=O)O1]

In exemplary embodiments, the compound is selected from:

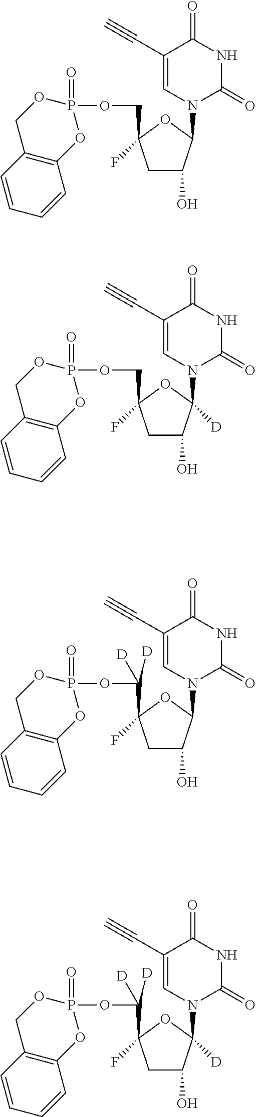 [SMILES: C#Cc1cn([C@@H]2O[C@](F)(COP3(=O)OCc4ccccc4O3)C[C@H]2O)c(=O)[nH]c1=O ; [2H][C@@]1(n2cc(C#C)c(=O)[nH]c2=O)O[C@](F)(COP2(=O)OCc3ccccc3O2)C[C@H]1O ; [2H]C([2H])(OP1(=O)OCc2ccccc2O1)[C@]1(F)C[C@@H](O)[C@H](n2cc(C#C)c(=O)[nH]c2=O)O1 ; [2H]C([2H])(OP1(=O)OCc2ccccc2O1)[C@]1(F)C[C@@H](O)[C@]([2H])(n2cc(C#C)c(=O)[nH]c2=O)O1]

In exemplary embodiments, the compound is selected from:

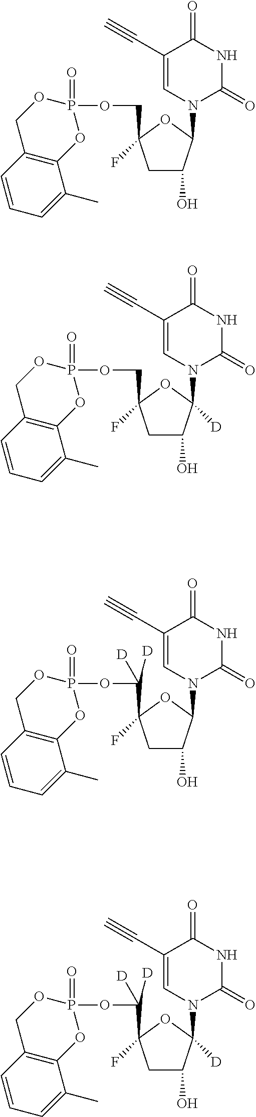 [SMILES: C#Cc1cn([C@@H]2O[C@](F)(COP3(=O)OCc4cccc(C)c4O3)C[C@H]2O)c(=O)[nH]c1=O ; [2H][C@@]1(n2cc(C#C)c(=O)[nH]c2=O)O[C@](F)(COP2(=O)OCc3cccc(C)c3O2)C[C@H]1O ; [2H]C([2H])(OP1(=O)OCc2cccc(C)c2O1)[C@]1(F)C[C@@H](O)[C@H](n2cc(C#C)c(=O)[nH]c2=O)O1 ; [2H]C([2H])(OP1(=O)OCc2cccc(C)c2O1)[C@]1(F)C[C@@H](O)[C@]([2H])(n2cc(C#C)c(=O)[nH]c2=O)O1]

In exemplary embodiments, the compound is selected from:

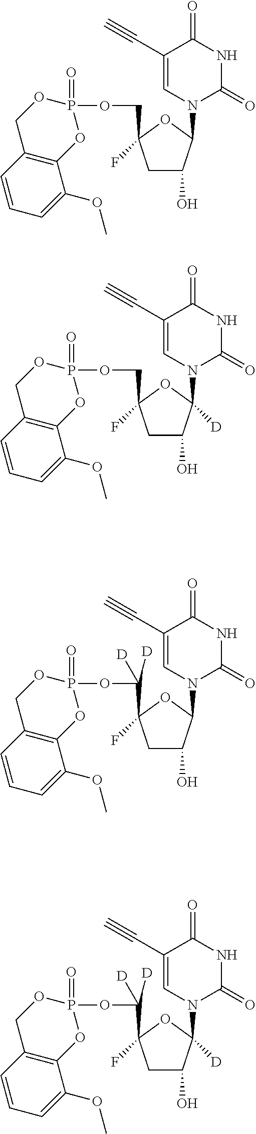 [SMILES: C#Cc1cn([C@@H]2O[C@](F)(COP3(=O)OCc4cccc(OC)c4O3)C[C@H]2O)c(=O)[nH]c1=O ; [2H][C@@]1(n2cc(C#C)c(=O)[nH]c2=O)O[C@](F)(COP2(=O)OCc3cccc(OC)c3O2)C[C@H]1O ; [2H]C([2H])(OP1(=O)OCc2cccc(OC)c2O1)[C@]1(F)C[C@@H](O)[C@H](n2cc(C#C)c(=O)[nH]c2=O)O1 ; [2H]C([2H])(OP1(=O)OCc2cccc(OC)c2O1)[C@]1(F)C[C@@H](O)[C@]([2H])(n2cc(C#C)c(=O)[nH]c2=O)O1]

In exemplary embodiments, the compound is selected from:

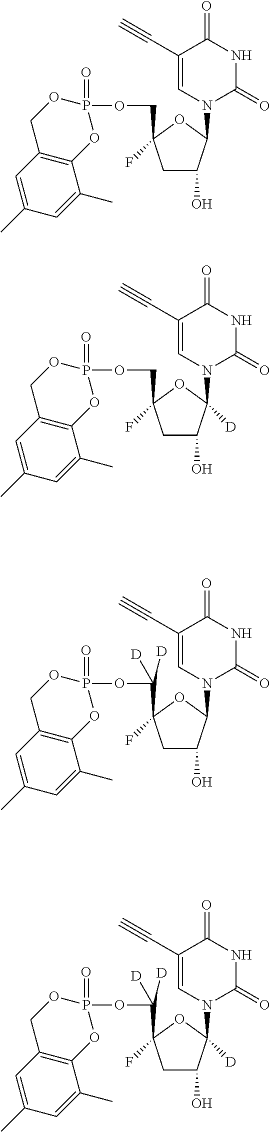 [SMILES: C#Cc1cn([C@@H]2O[C@](F)(COP3(=O)OCc4cc(C)cc(C)c4O3)C[C@H]2O)c(=O)[nH]c1=O ; [2H][C@@]1(n2cc(C#C)c(=O)[nH]c2=O)O[C@](F)(COP2(=O)OCc3cc(C)cc(C)c3O2)C[C@H]1O ; [2H]C([2H])(OP1(=O)OCc2cc(C)cc(C)c2O1)[C@]1(F)C[C@@H](O)[C@H](n2cc(C#C)c(=O)[nH]c2=O)O1 ; [2H]C([2H])(OP1(=O)OCc2cc(C)cc(C)c2O1)[C@]1(F)C[C@@H](O)[C@]([2H])(n2cc(C#C)c(=O)[nH]c2=O)O1]

In exemplary embodiments, the compound is selected from:

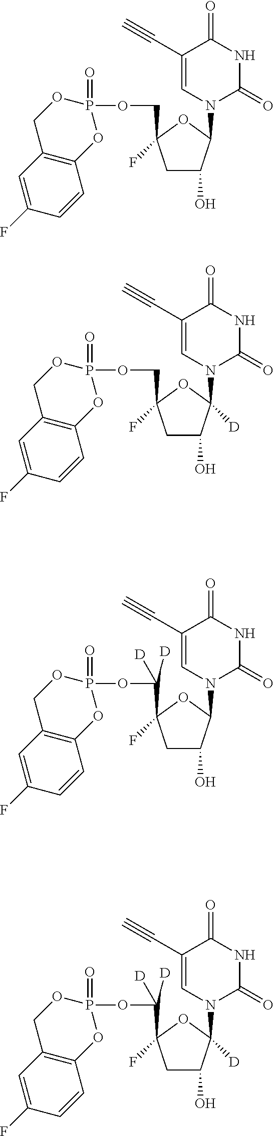 [SMILES: C#Cc1cn([C@@H]2O[C@](F)(COP3(=O)OCc4cc(F)ccc4O3)C[C@H]2O)c(=O)[nH]c1=O ; [2H][C@@]1(n2cc(C#C)c(=O)[nH]c2=O)O[C@](F)(COP2(=O)OCc3cc(F)ccc3O2)C[C@H]1O ; [2H]C([2H])(OP1(=O)OCc2cc(F)ccc2O1)[C@]1(F)C[C@@H](O)[C@H](n2cc(C#C)c(=O)[nH]c2=O)O1 ; [2H]C([2H])(OP1(=O)OCc2cc(F)ccc2O1)[C@]1(F)C[C@@H](O)[C@]([2H])(n2cc(C#C)c(=O)[nH]c2=O)O1]

In exemplary embodiments, the compound is selected from:

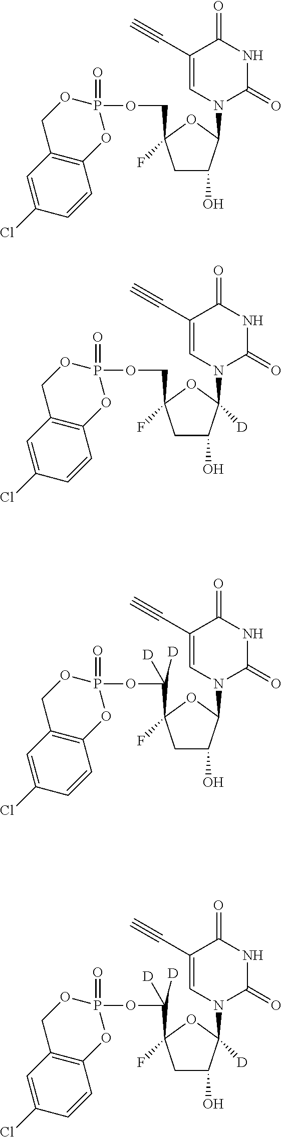 [SMILES: C#Cc1cn([C@@H]2O[C@](F)(COP3(=O)OCc4cc(Cl)ccc4O3)C[C@H]2O)c(=O)[nH]c1=O ; [2H][C@@]1(n2cc(C#C)c(=O)[nH]c2=O)O[C@](F)(COP2(=O)OCc3cc(Cl)ccc3O2)C[C@H]1O ; [2H]C([2H])(OP1(=O)OCc2cc(Cl)ccc2O1)[C@]1(F)C[C@@H](O)[C@H](n2cc(C#C)c(=O)[nH]c2=O)O1 ; [2H]C([2H])(OP1(=O)OCc2cc(Cl)ccc2O1)[C@]1(F)C[C@@H](O)[C@]([2H])(n2cc(C#C)c(=O)[nH]c2=O)O1]

In exemplary embodiments, the compound is selected from:

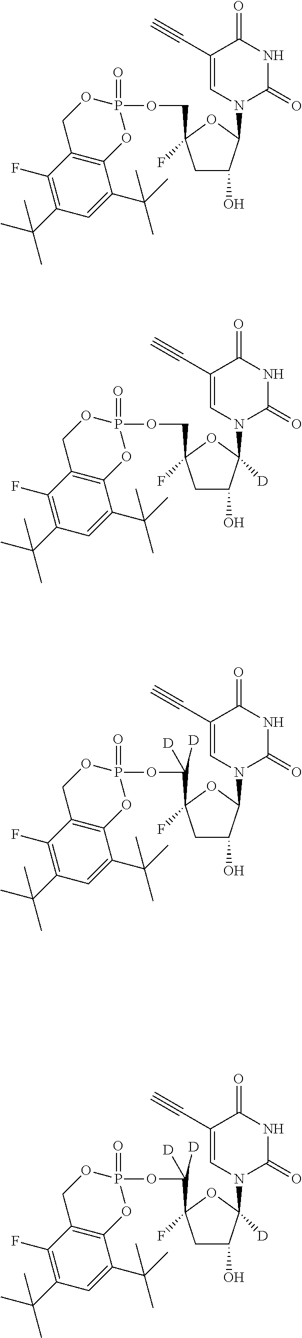 [SMILES: C#Cc1cn([C@@H]2O[C@](F)(COP3(=O)OCc4c(F)c(C(C)(C)C)cc(C(C)(C)C)c4O3)C[C@H]2O)c(=O)[nH]c1=O ; [2H][C@@]1(n2cc(C#C)c(=O)[nH]c2=O)O[C@](F)(COP2(=O)OCc3c(F)c(C(C)(C)C)cc(C(C)(C)C)c3O2)C[C@H]1O ; [2H]C([2H])(OP1(=O)OCc2c(F)c(C(C)(C)C)cc(C(C)(C)C)c2O1)[C@]1(F)C[C@@H](O)[C@H](n2cc(C#C)c(=O)[nH]c2=O)O1 ; [2H]C([2H])(OP1(=O)OCc2c(F)c(C(C)(C)C)cc(C(C)(C)C)c2O1)[C@]1(F)C[C@@H](O)[C@]([2H])(n2cc(C#C)c(=O)[nH]c2=O)O1]

In exemplary embodiments, the compound is selected from:

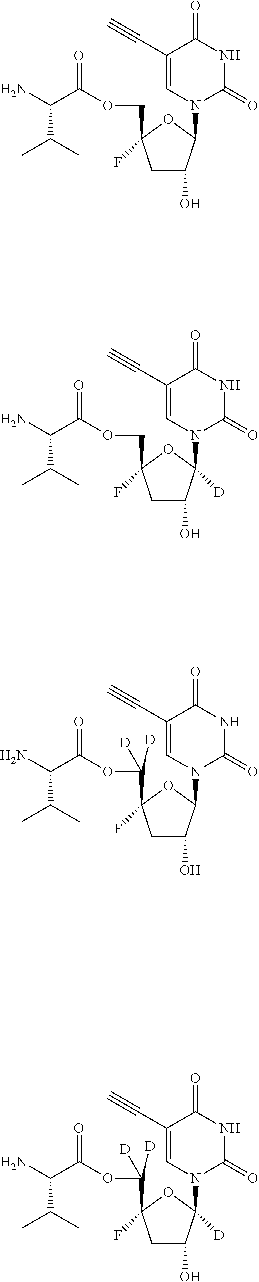 [SMILES: C#Cc1cn([C@@H]2O[C@](F)(COC(=O)[C@@H](N)C(C)C)C[C@H]2O)c(=O)[nH]c1=O ; [2H][C@@]1(n2cc(C#C)c(=O)[nH]c2=O)O[C@](F)(COC(=O)[C@@H](N)C(C)C)C[C@H]1O ; [2H]C([2H])(OC(=O)[C@@H](N)C(C)C)[C@]1(F)C[C@@H](O)[C@H](n2cc(C#C)c(=O)[nH]c2=O)O1 ; [2H]C([2H])(OC(=O)[C@@H](N)C(C)C)[C@]1(F)C[C@@H](O)[C@]([2H])(n2cc(C#C)c(=O)[nH]c2=O)O1]

In exemplary embodiments, the compound is selected from:

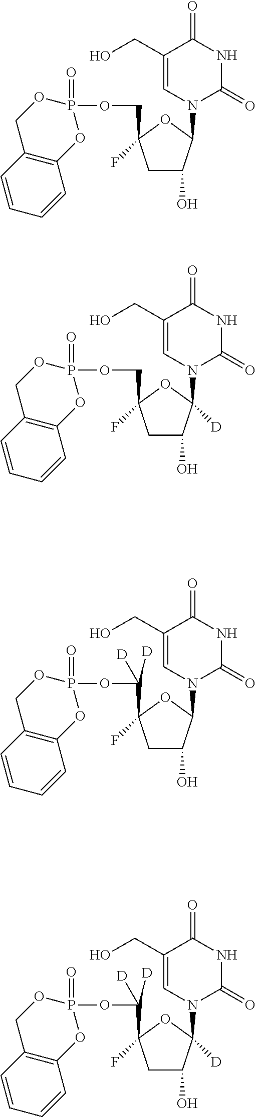 [SMILES: O=c1[nH]c(=O)n([C@@H]2O[C@](F)(COP3(=O)OCc4ccccc4O3)C[C@H]2O)cc1CO ; [2H][C@@]1(n2cc(CO)c(=O)[nH]c2=O)O[C@](F)(COP2(=O)OCc3ccccc3O2)C[C@H]1O ; [2H]C([2H])(OP1(=O)OCc2ccccc2O1)[C@]1(F)C[C@@H](O)[C@H](n2cc(CO)c(=O)[nH]c2=O)O1 ; [2H]C([2H])(OP1(=O)OCc2ccccc2O1)[C@]1(F)C[C@@H](O)[C@]([2H])(n2cc(CO)c(=O)[nH]c2=O)O1]

In exemplary embodiments, the compound is selected from:

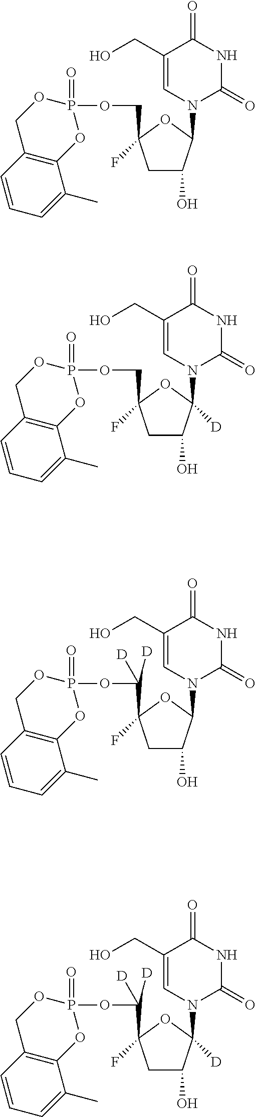 [SMILES: Cc1cccc2c1OP(=O)(OC[C@]1(F)C[C@@H](O)[C@H](n3cc(CO)c(=O)[nH]c3=O)O1)OC2 ; [2H][C@@]1(n2cc(CO)c(=O)[nH]c2=O)O[C@](F)(COP2(=O)OCc3cccc(C)c3O2)C[C@H]1O ; [2H]C([2H])(OP1(=O)OCc2cccc(C)c2O1)[C@]1(F)C[C@@H](O)[C@H](n2cc(CO)c(=O)[nH]c2=O)O1 ; [2H]C([2H])(OP1(=O)OCc2cccc(C)c2O1)[C@]1(F)C[C@@H](O)[C@]([2H])(n2cc(CO)c(=O)[nH]c2=O)O1]

In exemplary embodiments, the compound is selected from:

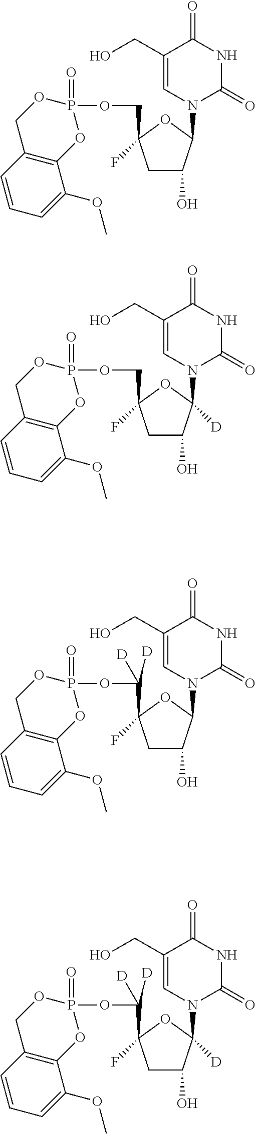 [SMILES: COc1cccc2c1OP(=O)(OC[C@]1(F)C[C@@H](O)[C@H](n3cc(CO)c(=O)[nH]c3=O)O1)OC2 ; [2H][C@@]1(n2cc(CO)c(=O)[nH]c2=O)O[C@](F)(COP2(=O)OCc3cccc(OC)c3O2)C[C@H]1O ; [2H]C([2H])(OP1(=O)OCc2cccc(OC)c2O1)[C@]1(F)C[C@@H](O)[C@H](n2cc(CO)c(=O)[nH]c2=O)O1 ; [2H]C([2H])(OP1(=O)OCc2cccc(OC)c2O1)[C@]1(F)C[C@@H](O)[C@]([2H])(n2cc(CO)c(=O)[nH]c2=O)O1]

In exemplary embodiments, the compound is selected from:

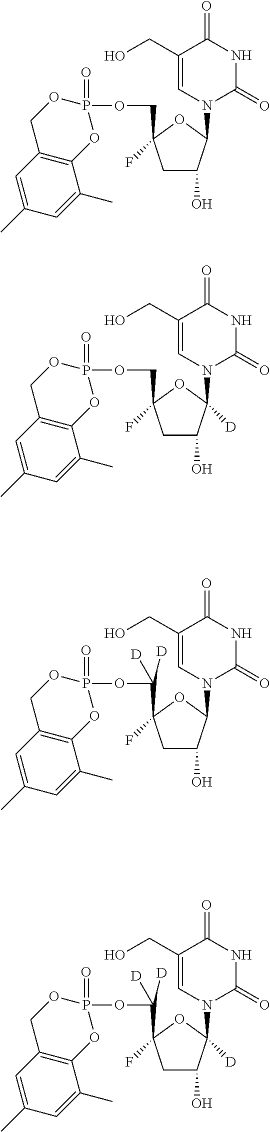 [SMILES: Cc1cc(C)c2c(c1)COP(=O)(OC[C@]1(F)C[C@@H](O)[C@H](n3cc(CO)c(=O)[nH]c3=O)O1)O2 ; [2H][C@@]1(n2cc(CO)c(=O)[nH]c2=O)O[C@](F)(COP2(=O)OCc3cc(C)cc(C)c3O2)C[C@H]1O ; [2H]C([2H])(OP1(=O)OCc2cc(C)cc(C)c2O1)[C@]1(F)C[C@@H](O)[C@H](n2cc(CO)c(=O)[nH]c2=O)O1 ; [2H]C([2H])(OP1(=O)OCc2cc(C)cc(C)c2O1)[C@]1(F)C[C@@H](O)[C@]([2H])(n2cc(CO)c(=O)[nH]c2=O)O1]

In exemplary embodiments, the compound is selected from:

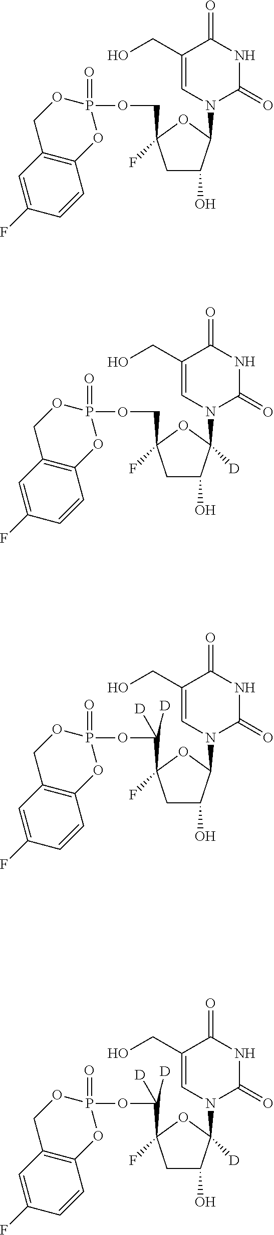 [SMILES: O=c1[nH]c(=O)n([C@@H]2O[C@](F)(COP3(=O)OCc4cc(F)ccc4O3)C[C@H]2O)cc1CO ; [2H][C@@]1(n2cc(CO)c(=O)[nH]c2=O)O[C@](F)(COP2(=O)OCc3cc(F)ccc3O2)C[C@H]1O ; [2H]C([2H])(OP1(=O)OCc2cc(F)ccc2O1)[C@]1(F)C[C@@H](O)[C@H](n2cc(CO)c(=O)[nH]c2=O)O1 ; [2H]C([2H])(OP1(=O)OCc2cc(F)ccc2O1)[C@]1(F)C[C@@H](O)[C@]([2H])(n2cc(CO)c(=O)[nH]c2=O)O1]

In exemplary embodiments, the compound is selected from:

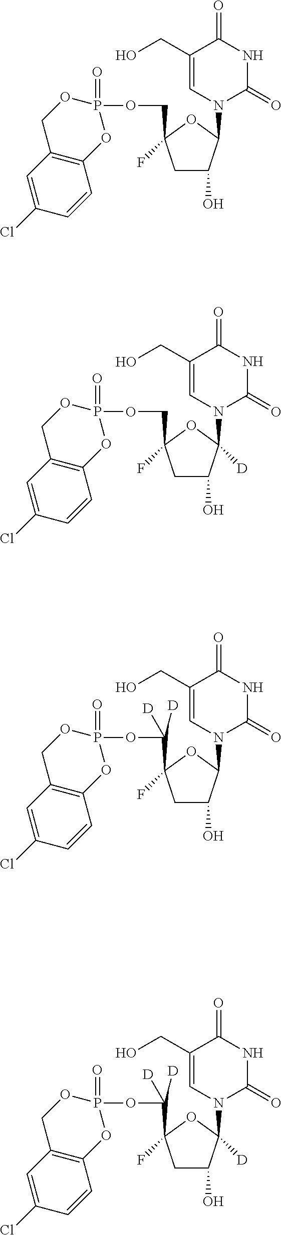 [SMILES: O=c1[nH]c(=O)n([C@@H]2O[C@](F)(COP3(=O)OCc4cc(Cl)ccc4O3)C[C@H]2O)cc1CO ; [2H][C@@]1(n2cc(CO)c(=O)[nH]c2=O)O[C@](F)(COP2(=O)OCc3cc(Cl)ccc3O2)C[C@H]1O ; [2H]C([2H])(OP1(=O)OCc2cc(Cl)ccc2O1)[C@]1(F)C[C@@H](O)[C@H](n2cc(CO)c(=O)[nH]c2=O)O1 ; [2H]C([2H])(OP1(=O)OCc2cc(Cl)ccc2O1)[C@]1(F)C[C@@H](O)[C@]([2H])(n2cc(CO)c(=O)[nH]c2=O)O1]

In exemplary embodiments, the compound is selected from:

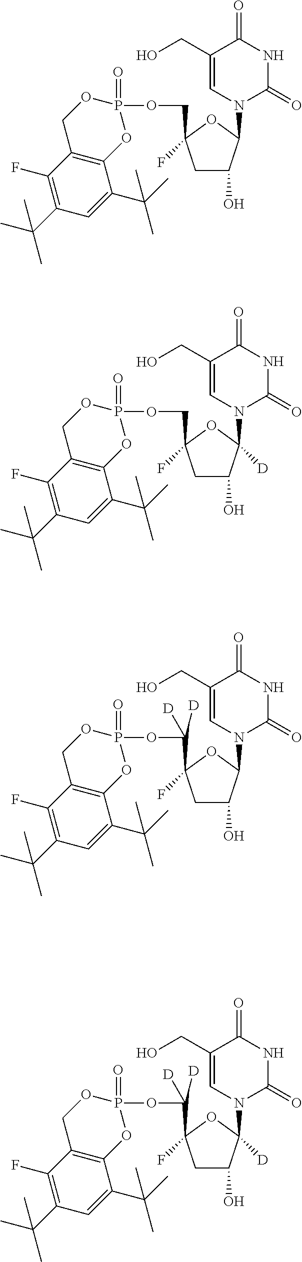 [SMILES: CC(C)(C)c1cc(C(C)(C)C)c2c(c1F)COP(=O)(OC[C@]1(F)C[C@@H](O)[C@H](n3cc(CO)c(=O)[nH]c3=O)O1)O2 ; [2H][C@@]1(n2cc(CO)c(=O)[nH]c2=O)O[C@](F)(COP2(=O)OCc3c(F)c(C(C)(C)C)cc(C(C)(C)C)c3O2)C[C@H]1O ; [2H]C([2H])(OP1(=O)OCc2c(F)c(C(C)(C)C)cc(C(C)(C)C)c2O1)[C@]1(F)C[C@@H](O)[C@H](n2cc(CO)c(=O)[nH]c2=O)O1 ; [2H]C([2H])(OP1(=O)OCc2c(F)c(C(C)(C)C)cc(C(C)(C)C)c2O1)[C@]1(F)C[C@@H](O)[C@]([2H])(n2cc(CO)c(=O)[nH]c2=O)O1]

In exemplary embodiments, the compound is selected from:

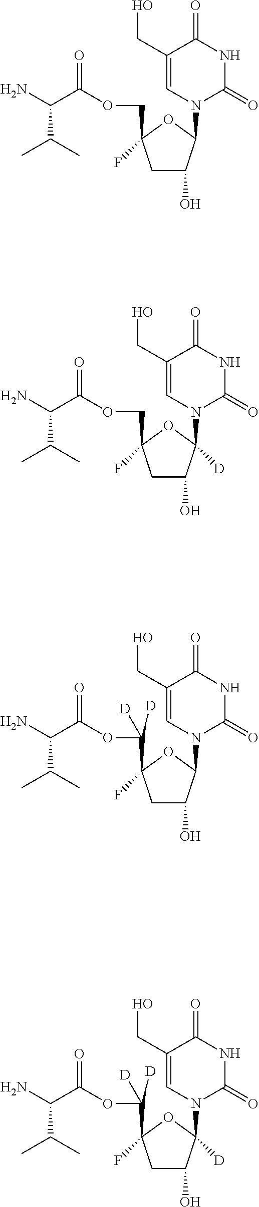 [SMILES: CC(C)[C@H](N)C(=O)OC[C@]1(F)C[C@@H](O)[C@H](n2cc(CO)c(=O)[nH]c2=O)O1 ; [2H][C@@]1(n2cc(CO)c(=O)[nH]c2=O)O[C@](F)(COC(=O)[C@@H](N)C(C)C)C[C@H]1O ; [2H]C([2H])(OC(=O)[C@@H](N)C(C)C)[C@]1(F)C[C@@H](O)[C@H](n2cc(CO)c(=O)[nH]c2=O)O1 ; [2H]C([2H])(OC(=O)[C@@H](N)C(C)C)[C@]1(F)C[C@@H](O)[C@]([2H])(n2cc(CO)c(=O)[nH]c2=O)O1]

In exemplary embodiments, the compound is selected from:

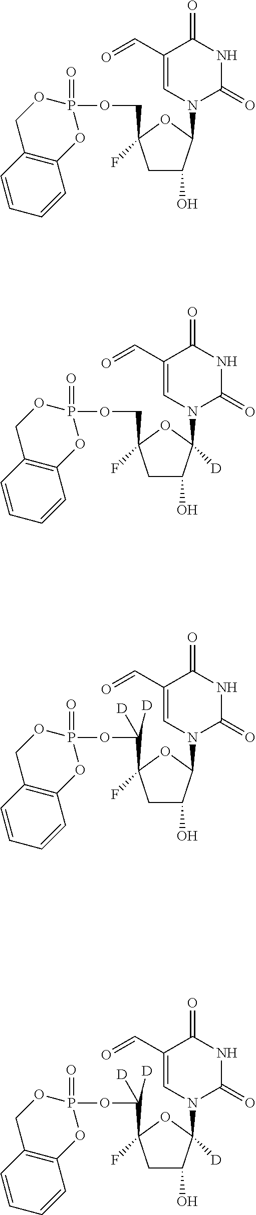 [SMILES: O=Cc1cn([C@@H]2O[C@](F)(COP3(=O)OCc4ccccc4O3)C[C@H]2O)c(=O)[nH]c1=O ; [2H][C@@]1(n2cc(C=O)c(=O)[nH]c2=O)O[C@](F)(COP2(=O)OCc3ccccc3O2)C[C@H]1O ; [2H]C([2H])(OP1(=O)OCc2ccccc2O1)[C@]1(F)C[C@@H](O)[C@H](n2cc(C=O)c(=O)[nH]c2=O)O1 ; [2H]C([2H])(OP1(=O)OCc2ccccc2O1)[C@]1(F)C[C@@H](O)[C@]([2H])(n2cc(C=O)c(=O)[nH]c2=O)O1]

In exemplary embodiments, the compound is selected from:

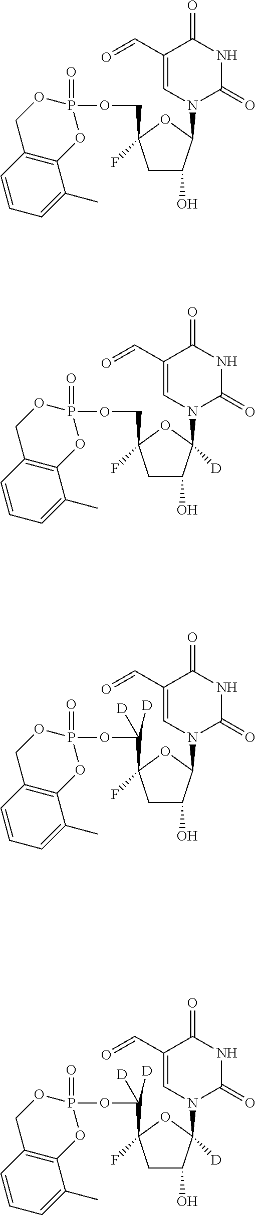 [SMILES: Cc1cccc2c1OP(=O)(OC[C@]1(F)C[C@@H](O)[C@H](n3cc(C=O)c(=O)[nH]c3=O)O1)OC2 ; [2H][C@@]1(n2cc(C=O)c(=O)[nH]c2=O)O[C@](F)(COP2(=O)OCc3cccc(C)c3O2)C[C@H]1O ; [2H]C([2H])(OP1(=O)OCc2cccc(C)c2O1)[C@]1(F)C[C@@H](O)[C@H](n2cc(C=O)c(=O)[nH]c2=O)O1 ; [2H]C([2H])(OP1(=O)OCc2cccc(C)c2O1)[C@]1(F)C[C@@H](O)[C@]([2H])(n2cc(C=O)c(=O)[nH]c2=O)O1]

In exemplary embodiments, the compound is selected from:

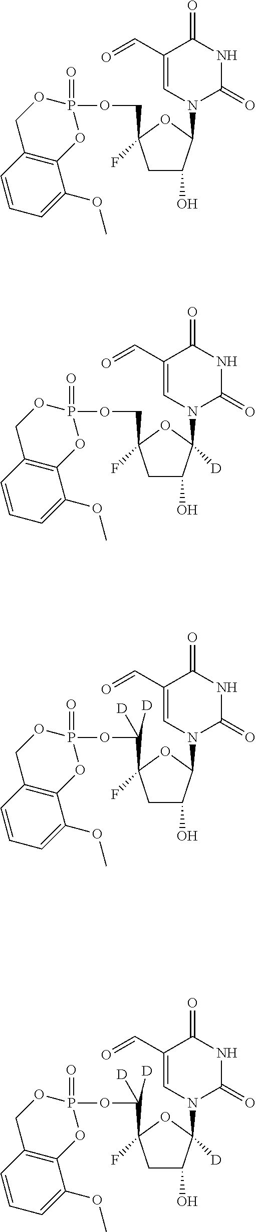 [SMILES: COc1cccc2c1OP(=O)(OC[C@]1(F)C[C@@H](O)[C@H](n3cc(C=O)c(=O)[nH]c3=O)O1)OC2 ; [2H][C@@]1(n2cc(C=O)c(=O)[nH]c2=O)O[C@](F)(COP2(=O)OCc3cccc(OC)c3O2)C[C@H]1O ; [2H]C([2H])(OP1(=O)OCc2cccc(OC)c2O1)[C@]1(F)C[C@@H](O)[C@H](n2cc(C=O)c(=O)[nH]c2=O)O1 ; [2H]C([2H])(OP1(=O)OCc2cccc(OC)c2O1)[C@]1(F)C[C@@H](O)[C@]([2H])(n2cc(C=O)c(=O)[nH]c2=O)O1]

In exemplary embodiments, the compound is selected from:

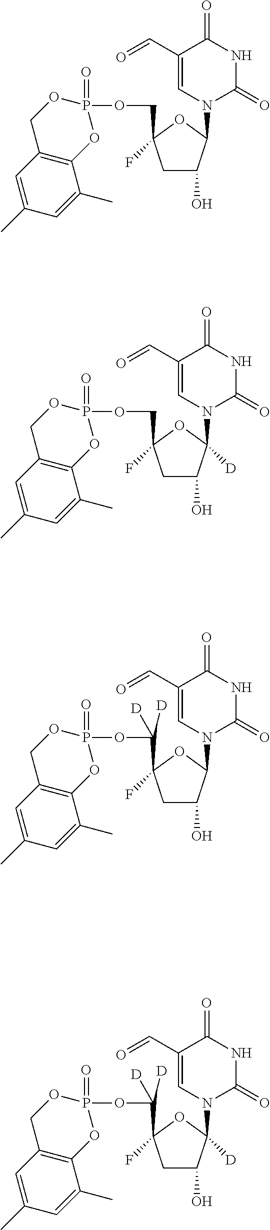 [SMILES: Cc1cc(C)c2c(c1)COP(=O)(OC[C@]1(F)C[C@@H](O)[C@H](n3cc(C=O)c(=O)[nH]c3=O)O1)O2 ; [2H][C@@]1(n2cc(C=O)c(=O)[nH]c2=O)O[C@](F)(COP2(=O)OCc3cc(C)cc(C)c3O2)C[C@H]1O ; [2H]C([2H])(OP1(=O)OCc2cc(C)cc(C)c2O1)[C@]1(F)C[C@@H](O)[C@H](n2cc(C=O)c(=O)[nH]c2=O)O1 ; [2H]C([2H])(OP1(=O)OCc2cc(C)cc(C)c2O1)[C@]1(F)C[C@@H](O)[C@]([2H])(n2cc(C=O)c(=O)[nH]c2=O)O1]

In exemplary embodiments, the compound is selected from:

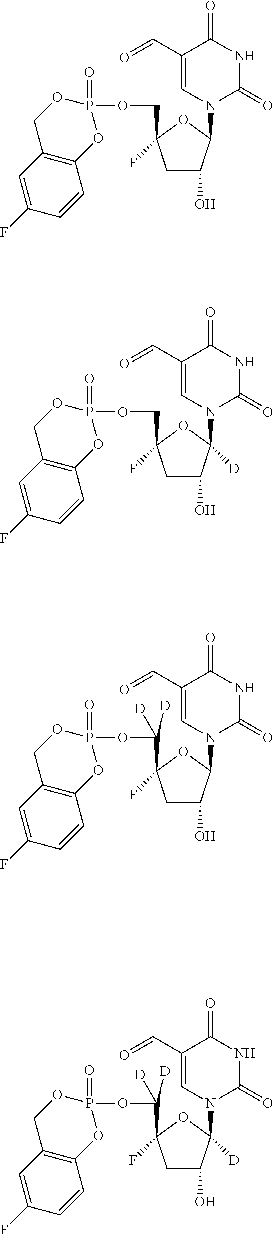 [SMILES: O=Cc1cn([C@@H]2O[C@](F)(COP3(=O)OCc4cc(F)ccc4O3)C[C@H]2O)c(=O)[nH]c1=O ; [2H][C@@]1(n2cc(C=O)c(=O)[nH]c2=O)O[C@](F)(COP2(=O)OCc3cc(F)ccc3O2)C[C@H]1O ; [2H]C([2H])(OP1(=O)OCc2cc(F)ccc2O1)[C@]1(F)C[C@@H](O)[C@H](n2cc(C=O)c(=O)[nH]c2=O)O1 ; [2H]C([2H])(OP1(=O)OCc2cc(F)ccc2O1)[C@]1(F)C[C@@H](O)[C@]([2H])(n2cc(C=O)c(=O)[nH]c2=O)O1]

In exemplary embodiments, the compound is selected from:

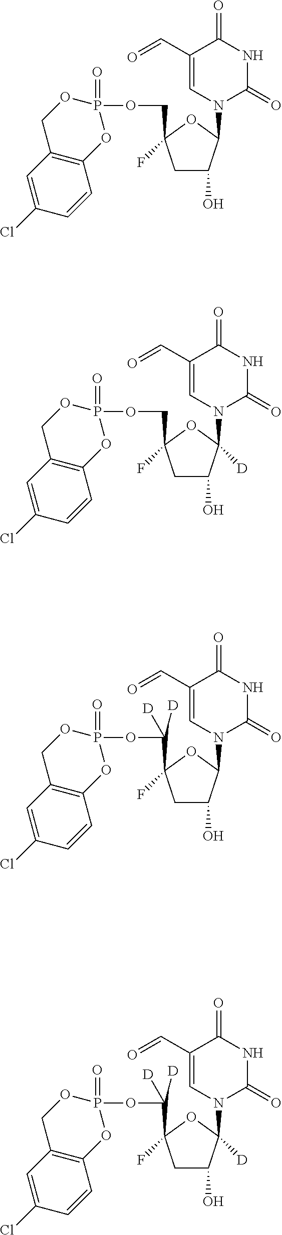 [SMILES: O=Cc1cn([C@@H]2O[C@](F)(COP3(=O)OCc4cc(Cl)ccc4O3)C[C@H]2O)c(=O)[nH]c1=O ; [2H][C@@]1(n2cc(C=O)c(=O)[nH]c2=O)O[C@](F)(COP2(=O)OCc3cc(Cl)ccc3O2)C[C@H]1O ; [2H]C([2H])(OP1(=O)OCc2cc(Cl)ccc2O1)[C@]1(F)C[C@@H](O)[C@H](n2cc(C=O)c(=O)[nH]c2=O)O1 ; [2H]C([2H])(OP1(=O)OCc2cc(Cl)ccc2O1)[C@]1(F)C[C@@H](O)[C@]([2H])(n2cc(C=O)c(=O)[nH]c2=O)O1]

In exemplary embodiments, the compound is selected from:

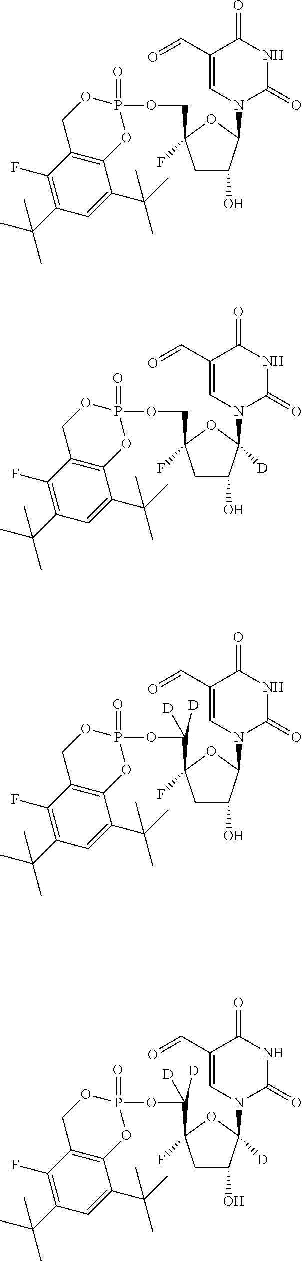 [SMILES: CC(C)(C)c1cc(C(C)(C)C)c2c(c1F)COP(=O)(OC[C@]1(F)C[C@@H](O)[C@H](n3cc(C=O)c(=O)[nH]c3=O)O1)O2 ; [2H][C@@]1(n2cc(C=O)c(=O)[nH]c2=O)O[C@](F)(COP2(=O)OCc3c(F)c(C(C)(C)C)cc(C(C)(C)C)c3O2)C[C@H]1O ; [2H]C([2H])(OP1(=O)OCc2c(F)c(C(C)(C)C)cc(C(C)(C)C)c2O1)[C@]1(F)C[C@@H](O)[C@H](n2cc(C=O)c(=O)[nH]c2=O)O1 ; [2H]C([2H])(OP1(=O)OCc2c(F)c(C(C)(C)C)cc(C(C)(C)C)c2O1)[C@]1(F)C[C@@H](O)[C@]([2H])(n2cc(C=O)c(=O)[nH]c2=O)O1]

In exemplary embodiments, the compound is selected from:

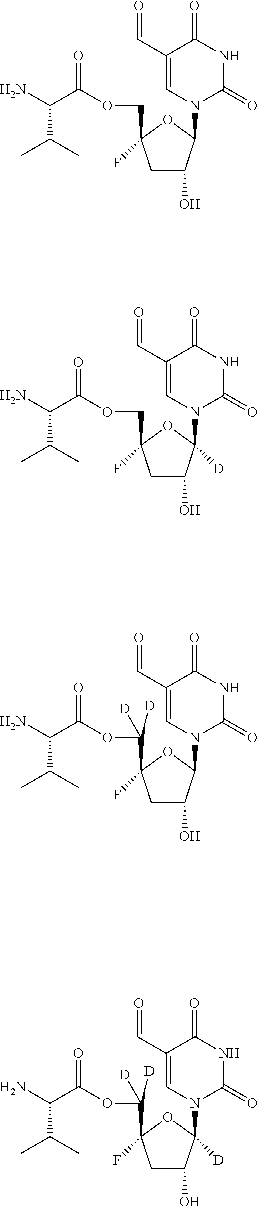 [SMILES: CC(C)[C@H](N)C(=O)OC[C@]1(F)C[C@@H](O)[C@H](n2cc(C=O)c(=O)[nH]c2=O)O1 ; [2H][C@@]1(n2cc(C=O)c(=O)[nH]c2=O)O[C@](F)(COC(=O)[C@@H](N)C(C)C)C[C@H]1O ; [2H]C([2H])(OC(=O)[C@@H](N)C(C)C)[C@]1(F)C[C@@H](O)[C@H](n2cc(C=O)c(=O)[nH]c2=O)O1 ; [2H]C([2H])(OC(=O)[C@@H](N)C(C)C)[C@]1(F)C[C@@H](O)[C@]([2H])(n2cc(C=O)c(=O)[nH]c2=O)O1]

In exemplary embodiments, the compound is selected from:

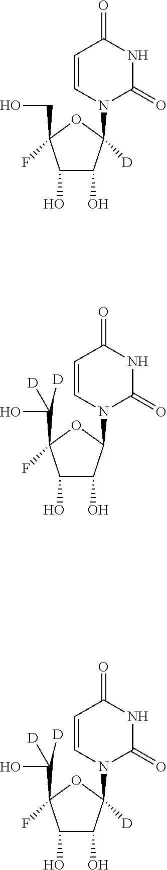 [SMILES: [2H][C@@]1(n2ccc(=O)[nH]c2=O)O[C@](F)(CO)[C@@H](O)[C@H]1O ; [2H]C([2H])(O)[C@@]1(F)O[C@@H](n2ccc(=O)[nH]c2=O)[C@H](O)[C@@H]1O ; [2H]C([2H])(O)[C@@]1(F)O[C@@]([2H])(n2ccc(=O)[nH]c2=O)[C@H](O)[C@@H]1O]

In exemplary embodiments, the compound is selected from:

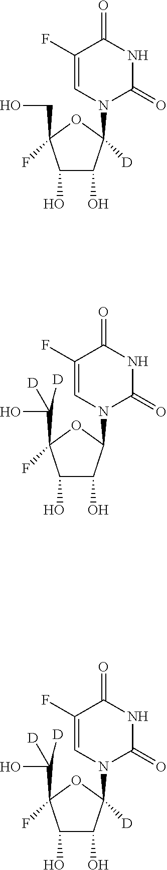 [SMILES: [2H][C@@]1(n2cc(F)c(=O)[nH]c2=O)O[C@](F)(CO)[C@@H](O)[C@H]1O ; [2H]C([2H])(O)[C@@]1(F)O[C@@H](n2cc(F)c(=O)[nH]c2=O)[C@H](O)[C@@H]1O ; [2H]C([2H])(O)[C@@]1(F)O[C@@]([2H])(n2cc(F)c(=O)[nH]c2=O)[C@H](O)[C@@H]1O]

In exemplary embodiments, the compound is selected from:

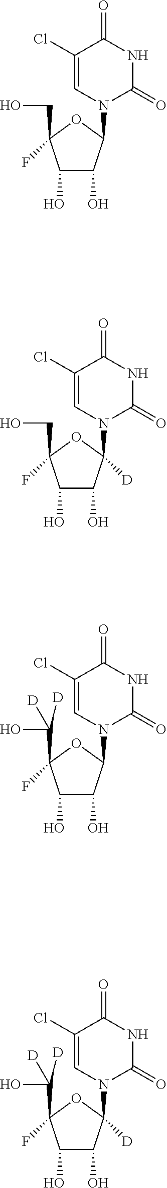 [SMILES: O=c1[nH]c(=O)n([C@@H]2O[C@](F)(CO)[C@@H](O)[C@H]2O)cc1Cl ; [2H][C@@]1(n2cc(Cl)c(=O)[nH]c2=O)O[C@](F)(CO)[C@@H](O)[C@H]1O ; [2H]C([2H])(O)[C@@]1(F)O[C@@H](n2cc(Cl)c(=O)[nH]c2=O)[C@H](O)[C@@H]1O ; [2H]C([2H])(O)[C@@]1(F)O[C@@]([2H])(n2cc(Cl)c(=O)[nH]c2=O)[C@H](O)[C@@H]1O]

In exemplary embodiments, the compound is selected from:

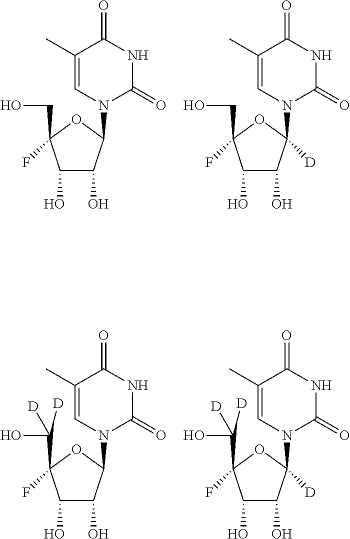

In exemplary embodiments, the compound is selected from:

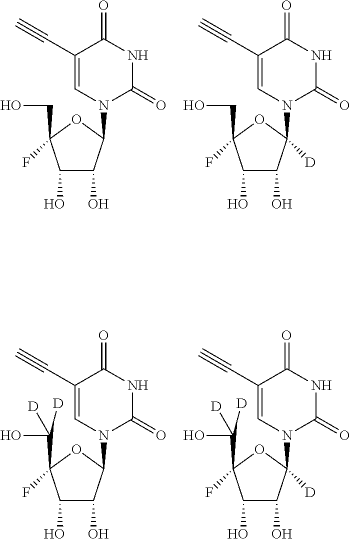

In exemplary embodiments, the compound is selected from:

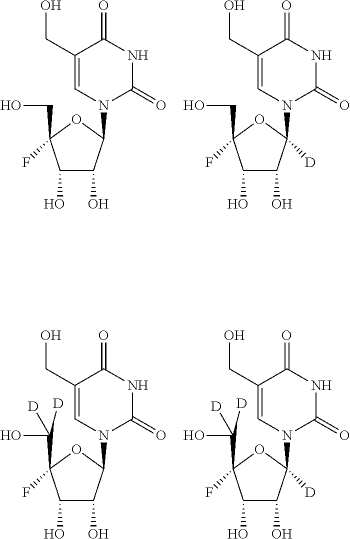

In exemplary embodiments, the compound is selected from:

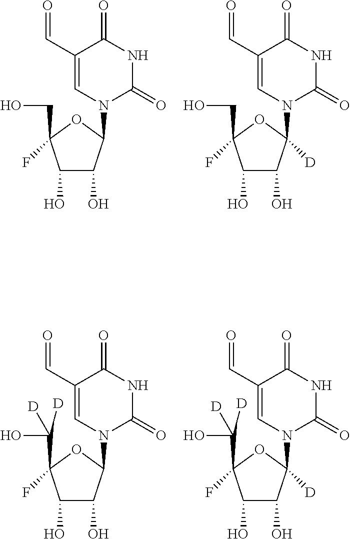

In exemplary embodiments, the compound is selected from:

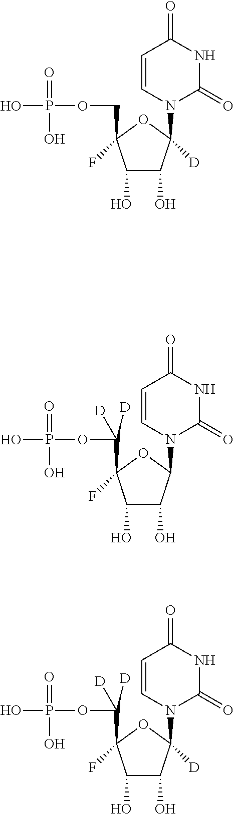 [SMILES: [2H][C@@]1(n2ccc(=O)[nH]c2=O)O[C@](F)(COP(=O)(O)O)[C@@H](O)[C@H]1O ; [2H]C([2H])(OP(=O)(O)O)[C@@]1(F)O[C@@H](n2ccc(=O)[nH]c2=O)[C@H](O)[C@@H]1O ; [2H]C([2H])(OP(=O)(O)O)[C@@]1(F)O[C@@]([2H])(n2ccc(=O)[nH]c2=O)[C@H](O)[C@@H]1O]

In exemplary embodiments, the compound is selected from:

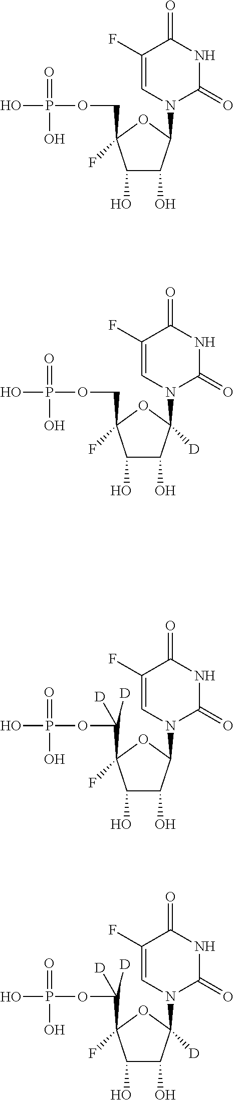 [SMILES: O=c1[nH]c(=O)n([C@@H]2O[C@](F)(COP(=O)(O)O)[C@@H](O)[C@H]2O)cc1F ; [2H][C@@]1(n2cc(F)c(=O)[nH]c2=O)O[C@](F)(COP(=O)(O)O)[C@@H](O)[C@H]1O ; [2H]C([2H])(OP(=O)(O)O)[C@@]1(F)O[C@@H](n2cc(F)c(=O)[nH]c2=O)[C@H](O)[C@@H]1O ; [2H]C([2H])(OP(=O)(O)O)[C@@]1(F)O[C@@]([2H])(n2cc(F)c(=O)[nH]c2=O)[C@H](O)[C@@H]1O]

In exemplary embodiments, the compound is selected from:

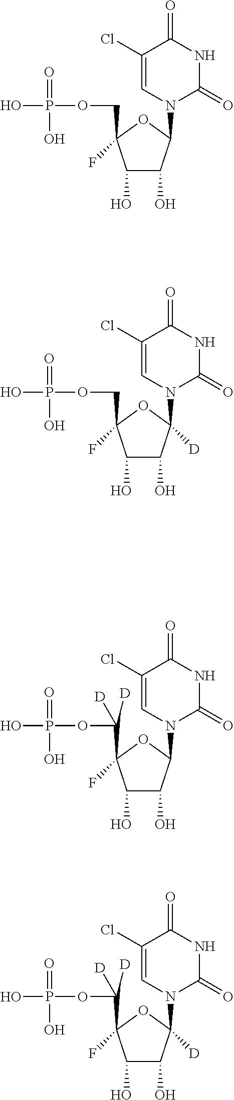 [SMILES: O=c1[nH]c(=O)n([C@@H]2O[C@](F)(COP(=O)(O)O)[C@@H](O)[C@H]2O)cc1Cl ; [2H][C@@]1(n2cc(Cl)c(=O)[nH]c2=O)O[C@](F)(COP(=O)(O)O)[C@@H](O)[C@H]1O ; [2H]C([2H])(OP(=O)(O)O)[C@@]1(F)O[C@@H](n2cc(Cl)c(=O)[nH]c2=O)[C@H](O)[C@@H]1O ; [2H]C([2H])(OP(=O)(O)O)[C@@]1(F)O[C@@]([2H])(n2cc(Cl)c(=O)[nH]c2=O)[C@H](O)[C@@H]1O]

In exemplary embodiments, the compound is selected from:

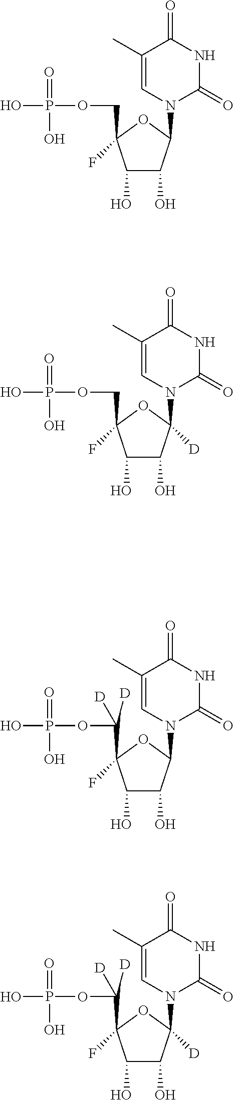 [SMILES: Cc1cn([C@@H]2O[C@](F)(COP(=O)(O)O)[C@@H](O)[C@H]2O)c(=O)[nH]c1=O ; [2H][C@@]1(n2cc(C)c(=O)[nH]c2=O)O[C@](F)(COP(=O)(O)O)[C@@H](O)[C@H]1O ; [2H]C([2H])(OP(=O)(O)O)[C@@]1(F)O[C@@H](n2cc(C)c(=O)[nH]c2=O)[C@H](O)[C@@H]1O ; [2H]C([2H])(OP(=O)(O)O)[C@@]1(F)O[C@@]([2H])(n2cc(C)c(=O)[nH]c2=O)[C@H](O)[C@@H]1O]

In exemplary embodiments, the compound is selected from:

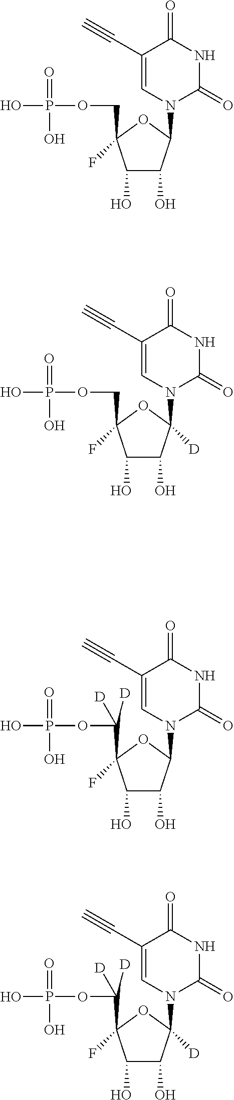 [SMILES: C#Cc1cn([C@@H]2O[C@](F)(COP(=O)(O)O)[C@@H](O)[C@H]2O)c(=O)[nH]c1=O ; [2H][C@@]1(n2cc(C#C)c(=O)[nH]c2=O)O[C@](F)(COP(=O)(O)O)[C@@H](O)[C@H]1O ; [2H]C([2H])(OP(=O)(O)O)[C@@]1(F)O[C@@H](n2cc(C#C)c(=O)[nH]c2=O)[C@H](O)[C@@H]1O ; [2H]C([2H])(OP(=O)(O)O)[C@@]1(F)O[C@@]([2H])(n2cc(C#C)c(=O)[nH]c2=O)[C@H](O)[C@@H]1O]

In exemplary embodiments, the compound is selected from:

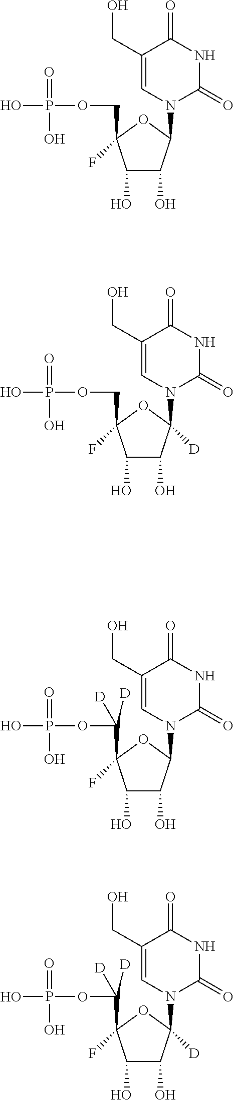 [SMILES: O=c1[nH]c(=O)n([C@@H]2O[C@](F)(COP(=O)(O)O)[C@@H](O)[C@H]2O)cc1CO ; [2H][C@@]1(n2cc(CO)c(=O)[nH]c2=O)O[C@](F)(COP(=O)(O)O)[C@@H](O)[C@H]1O ; [2H]C([2H])(OP(=O)(O)O)[C@@]1(F)O[C@@H](n2cc(CO)c(=O)[nH]c2=O)[C@H](O)[C@@H]1O ; [2H]C([2H])(OP(=O)(O)O)[C@@]1(F)O[C@@]([2H])(n2cc(CO)c(=O)[nH]c2=O)[C@H](O)[C@@H]1O]

In exemplary embodiments, the compound is selected from:

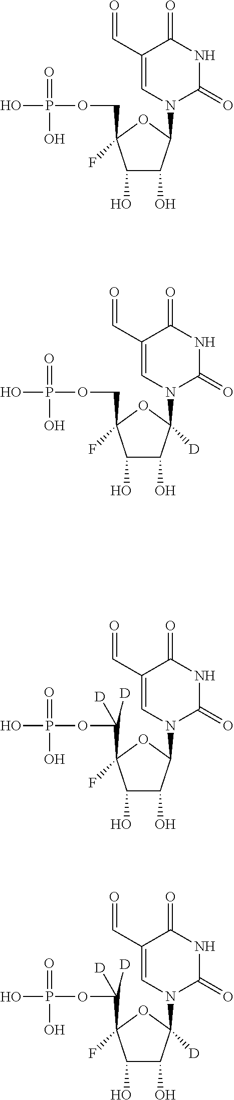 [SMILES: O=Cc1cn([C@@H]2O[C@](F)(COP(=O)(O)O)[C@@H](O)[C@H]2O)c(=O)[nH]c1=O ; [2H][C@@]1(n2cc(C=O)c(=O)[nH]c2=O)O[C@](F)(COP(=O)(O)O)[C@@H](O)[C@H]1O ; [2H]C([2H])(OP(=O)(O)O)[C@@]1(F)O[C@@H](n2cc(C=O)c(=O)[nH]c2=O)[C@H](O)[C@@H]1O ; [2H]C([2H])(OP(=O)(O)O)[C@@]1(F)O[C@@]([2H])(n2cc(C=O)c(=O)[nH]c2=O)[C@H](O)[C@@H]1O]

In exemplary embodiments, the compound is selected from:

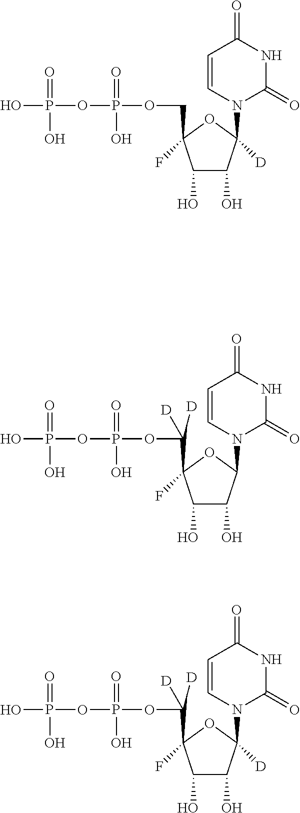 [SMILES: [2H][C@@]1(n2ccc(=O)[nH]c2=O)O[C@](F)(COP(=O)(O)OP(=O)(O)O)[C@@H](O)[C@H]1O ; [2H]C([2H])(OP(=O)(O)OP(=O)(O)O)[C@@]1(F)O[C@@H](n2ccc(=O)[nH]c2=O)[C@H](O)[C@@H]1O ; [2H]C([2H])(OP(=O)(O)OP(=O)(O)O)[C@@]1(F)O[C@@]([2H])(n2ccc(=O)[nH]c2=O)[C@H](O)[C@@H]1O]

In exemplary embodiments, the compound is selected from:

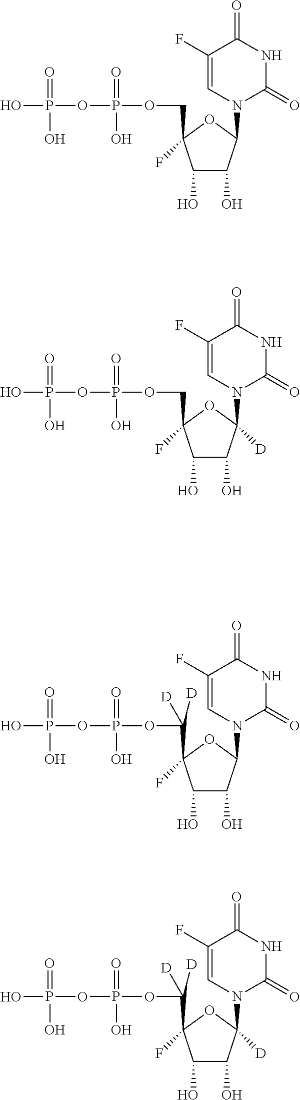 [SMILES: O=c1[nH]c(=O)n([C@@H]2O[C@](F)(COP(=O)(O)OP(=O)(O)O)[C@@H](O)[C@H]2O)cc1F ; [2H][C@@]1(n2cc(F)c(=O)[nH]c2=O)O[C@](F)(COP(=O)(O)OP(=O)(O)O)[C@@H](O)[C@H]1O ; [2H]C([2H])(OP(=O)(O)OP(=O)(O)O)[C@@]1(F)O[C@@H](n2cc(F)c(=O)[nH]c2=O)[C@H](O)[C@@H]1O ; [2H]C([2H])(OP(=O)(O)OP(=O)(O)O)[C@@]1(F)O[C@@]([2H])(n2cc(F)c(=O)[nH]c2=O)[C@H](O)[C@@H]1O]

In exemplary embodiments, the compound is selected from:

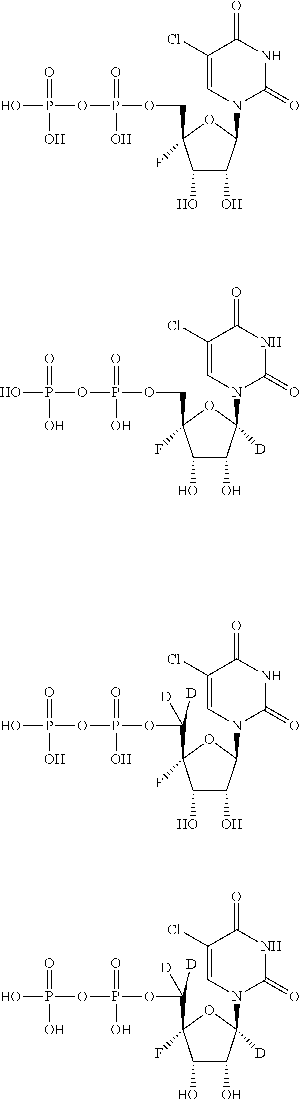 [SMILES: O=c1[nH]c(=O)n([C@@H]2O[C@](F)(COP(=O)(O)OP(=O)(O)O)[C@@H](O)[C@H]2O)cc1Cl ; [2H][C@@]1(n2cc(Cl)c(=O)[nH]c2=O)O[C@](F)(COP(=O)(O)OP(=O)(O)O)[C@@H](O)[C@H]1O ; [2H]C([2H])(OP(=O)(O)OP(=O)(O)O)[C@@]1(F)O[C@@H](n2cc(Cl)c(=O)[nH]c2=O)[C@H](O)[C@@H]1O ; [2H]C([2H])(OP(=O)(O)OP(=O)(O)O)[C@@]1(F)O[C@@]([2H])(n2cc(Cl)c(=O)[nH]c2=O)[C@H](O)[C@@H]1O]

In exemplary embodiments, the compound is selected from:

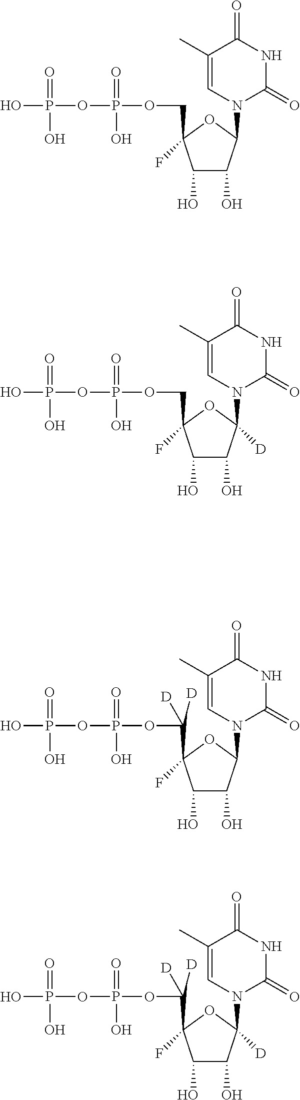 [SMILES: Cc1cn([C@@H]2O[C@](F)(COP(=O)(O)OP(=O)(O)O)[C@@H](O)[C@H]2O)c(=O)[nH]c1=O ; [2H][C@@]1(n2cc(C)c(=O)[nH]c2=O)O[C@](F)(COP(=O)(O)OP(=O)(O)O)[C@@H](O)[C@H]1O ; [2H]C([2H])(OP(=O)(O)OP(=O)(O)O)[C@@]1(F)O[C@@H](n2cc(C)c(=O)[nH]c2=O)[C@H](O)[C@@H]1O ; [2H]C([2H])(OP(=O)(O)OP(=O)(O)O)[C@@]1(F)O[C@@]([2H])(n2cc(C)c(=O)[nH]c2=O)[C@H](O)[C@@H]1O]

In exemplary embodiments, the compound is selected from:

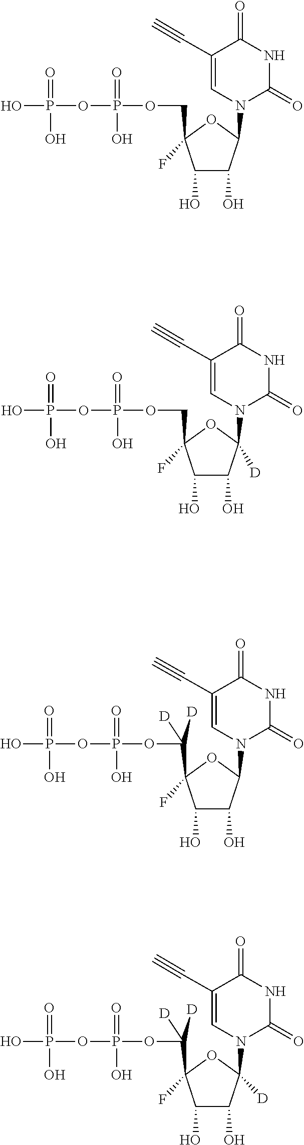

In exemplary embodiments, the compound is selected from:

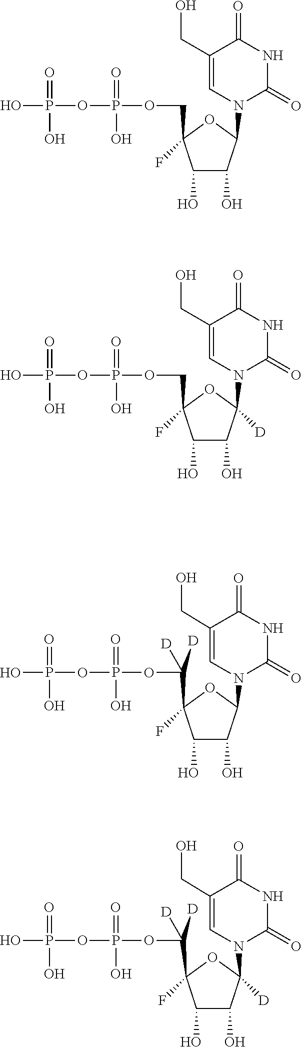

In exemplary embodiments, the compound is selected from:

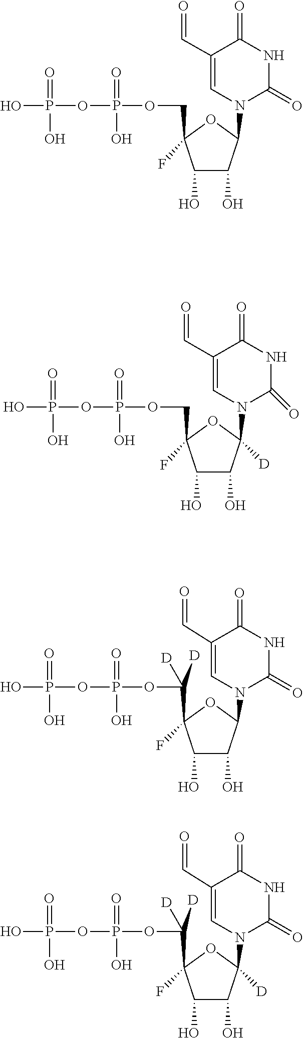

In exemplary embodiments, the compound is selected from:

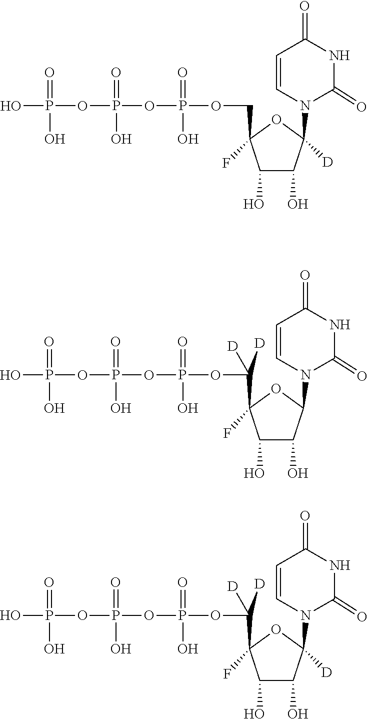

In exemplary embodiments, the compound is selected from:

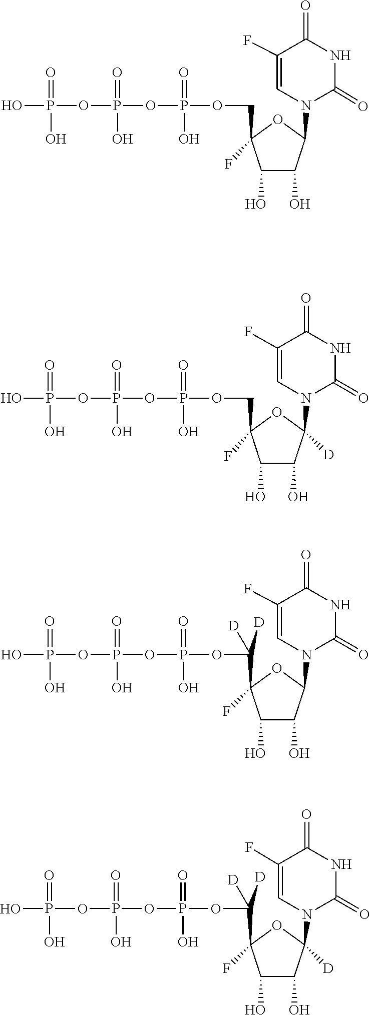

In exemplary embodiments, the compound is selected from:

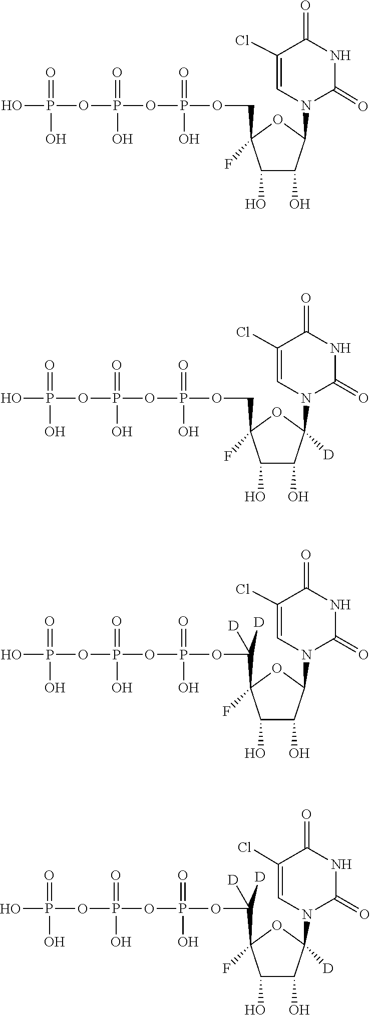

In exemplary embodiments, the compound is selected from:

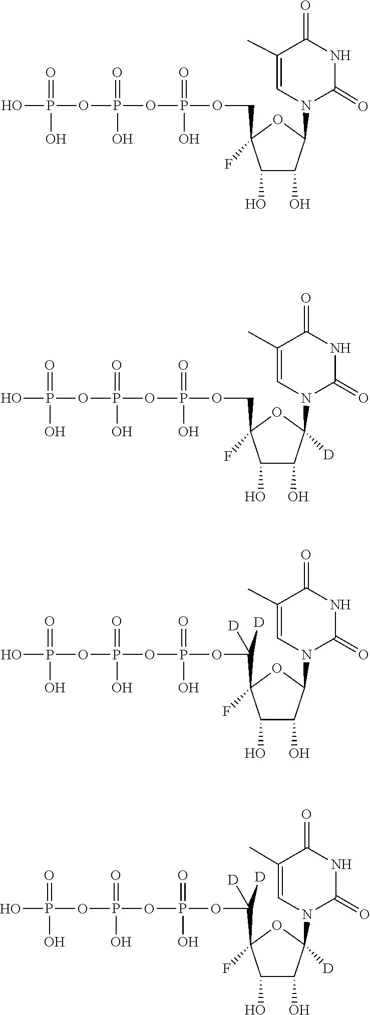

In exemplary embodiments, the compound is selected from:

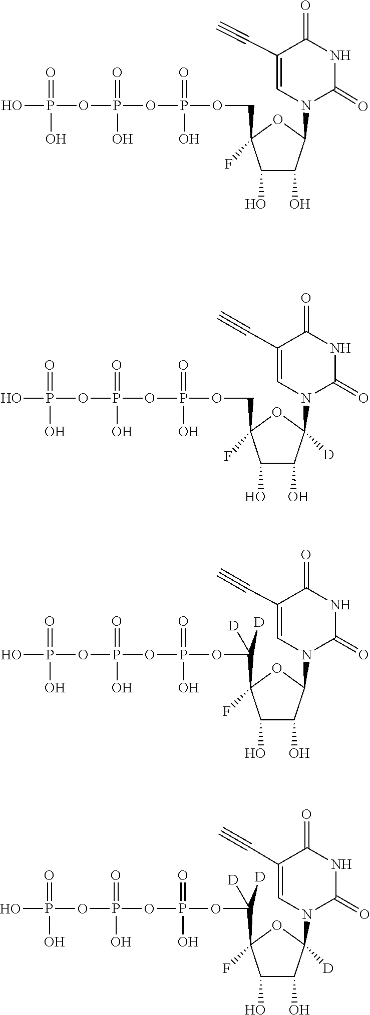

In exemplary embodiments, the compound is selected from:

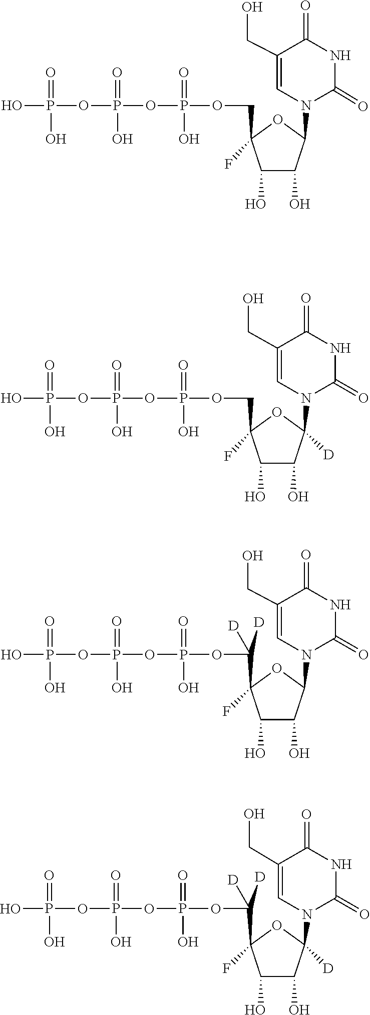

In exemplary embodiments, the compound is selected from:

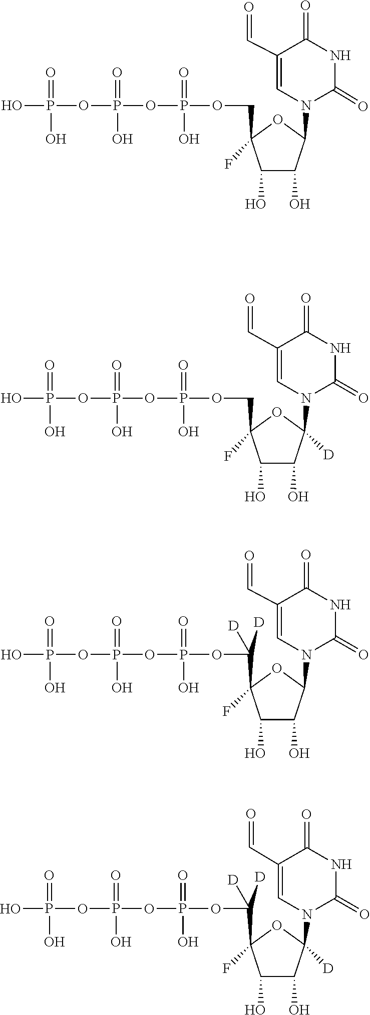

In exemplary embodiments, the compound is selected from:

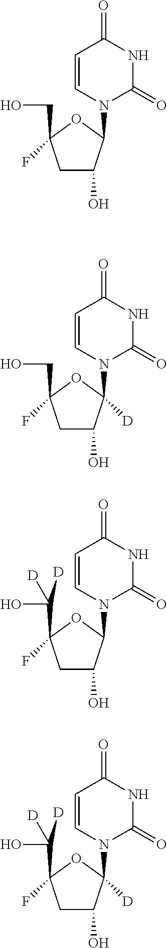

In exemplary embodiments, the compound is selected from:

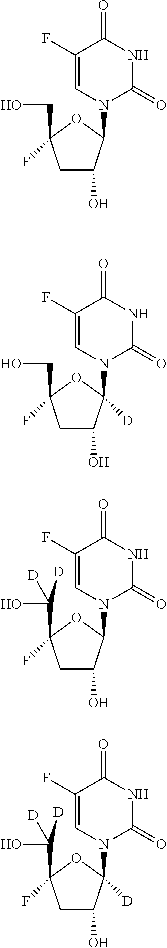

In exemplary embodiments, the compound is selected from:

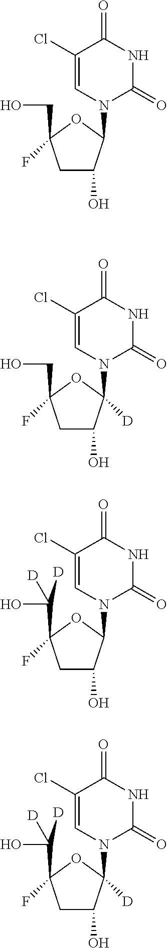

In exemplary embodiments, the compound is selected from:

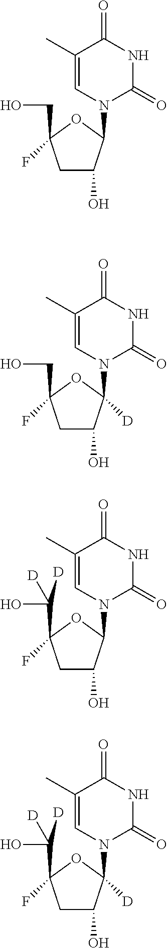

In exemplary embodiments, the compound is selected from:

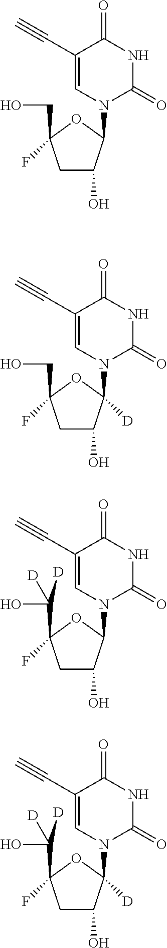

In exemplary embodiments, the compound is selected from:

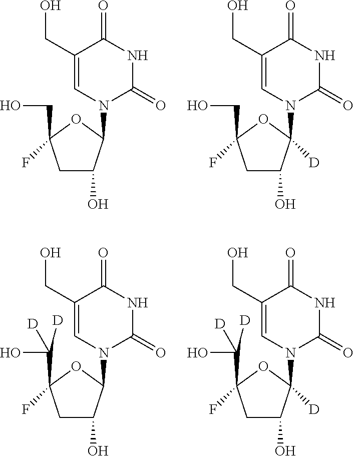

In exemplary embodiments, the compound is selected from:

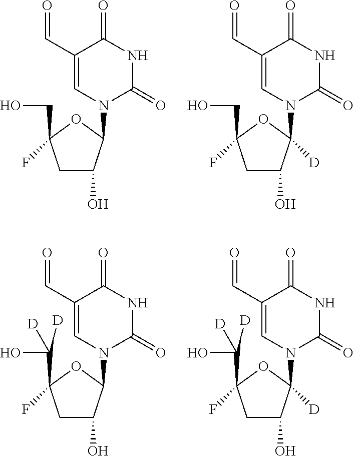

In exemplary embodiments, the compound is selected from:

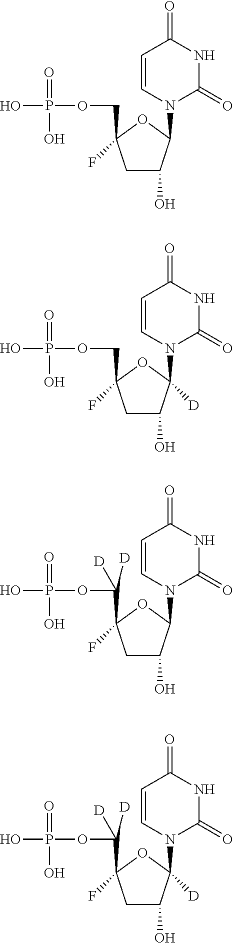

In exemplary embodiments, the compound is selected from:

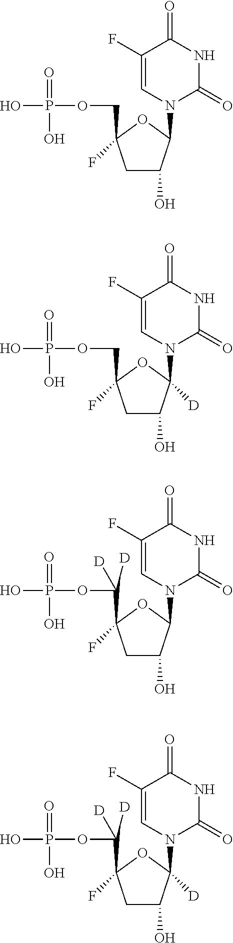

In exemplary embodiments, the compound is selected from:

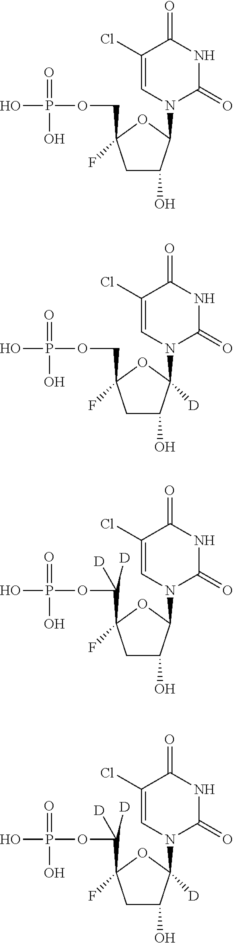

In exemplary embodiments, the compound is selected from:

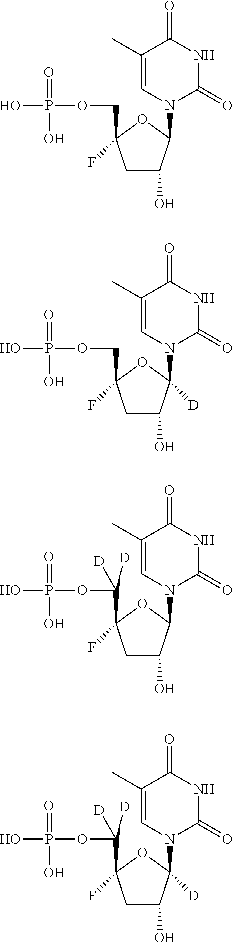

In exemplary embodiments, the compound is selected from:

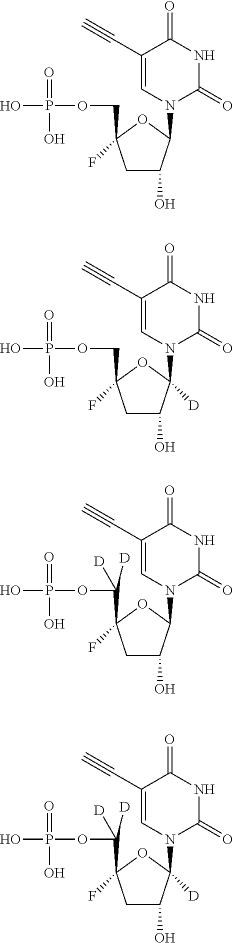

In exemplary embodiments, the compound is selected from:

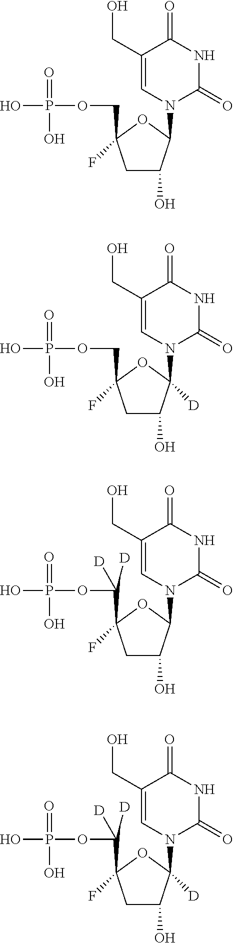

In exemplary embodiments, the compound is selected from:

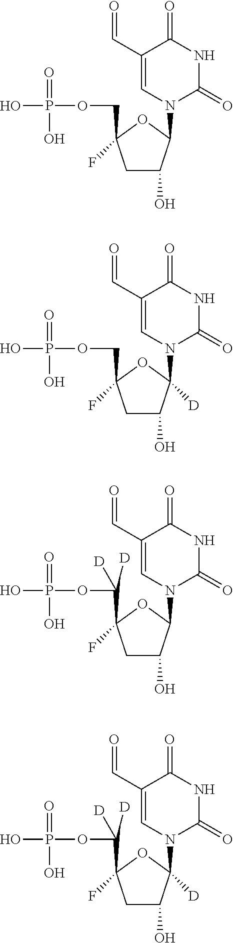

In exemplary embodiments, the compound is selected from:

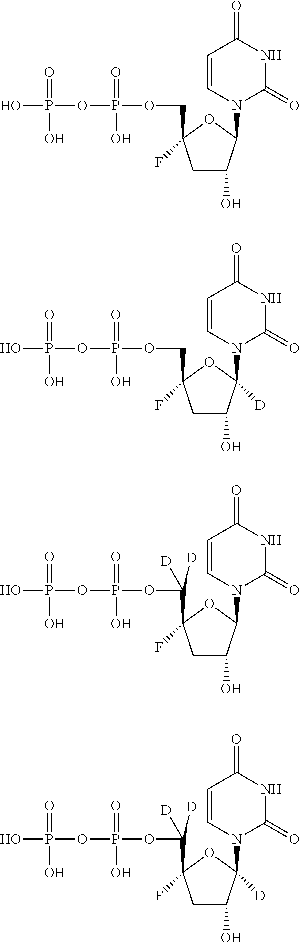

In exemplary embodiments, the compound is selected from:

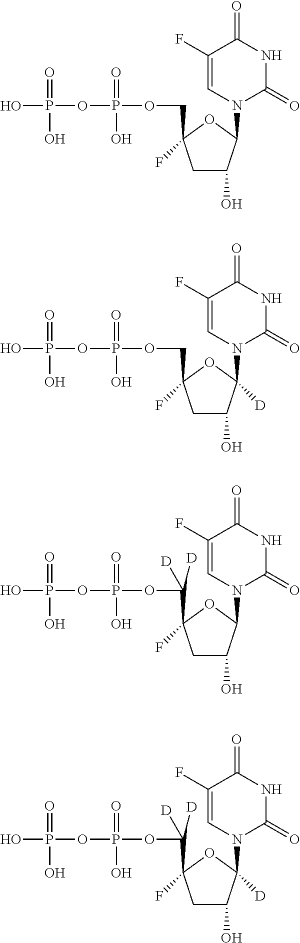

In exemplary embodiments, the compound is selected from:

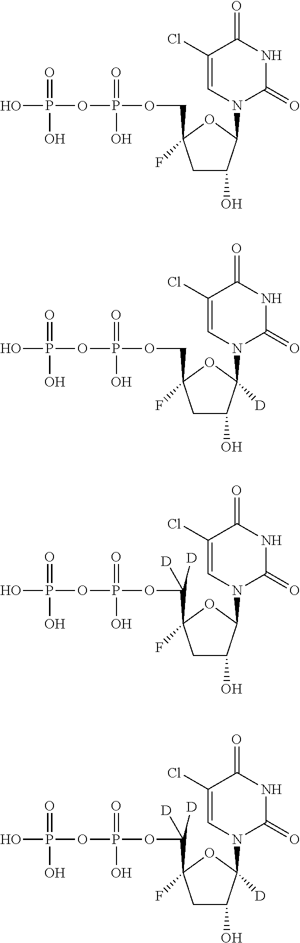

In exemplary embodiments, the compound is selected from:

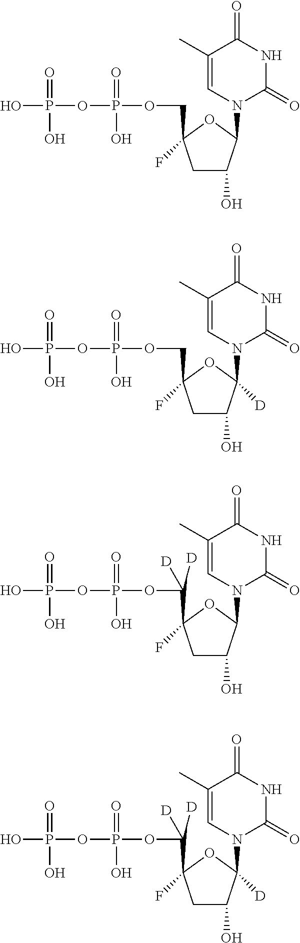

In exemplary embodiments, the compound is selected from:

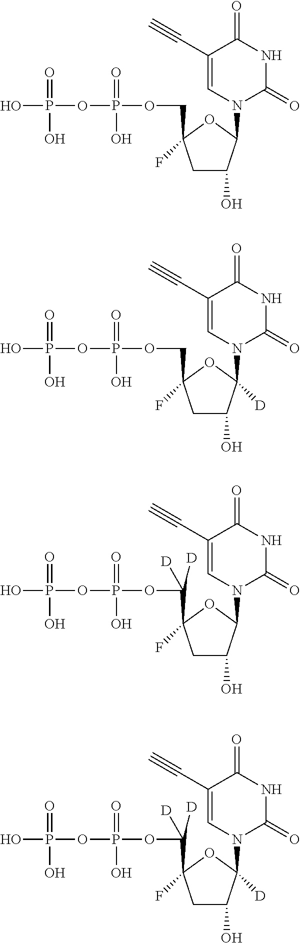

In exemplary embodiments, the compound is selected from:

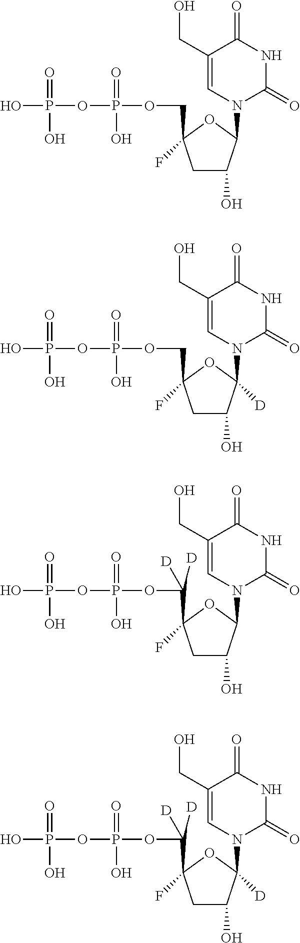

In exemplary embodiments, the compound is selected from:

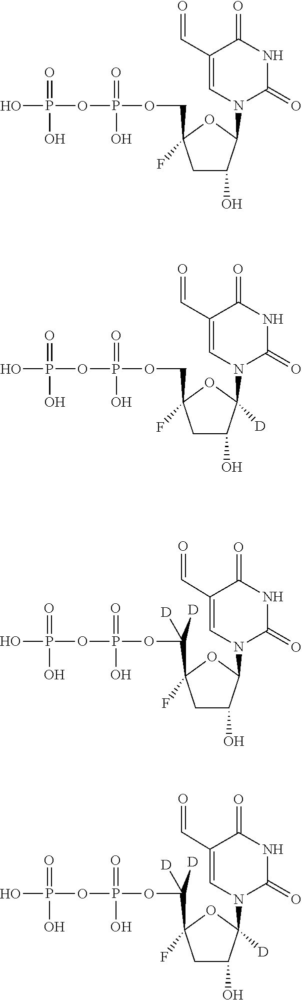

In exemplary embodiments, the compound is selected from:

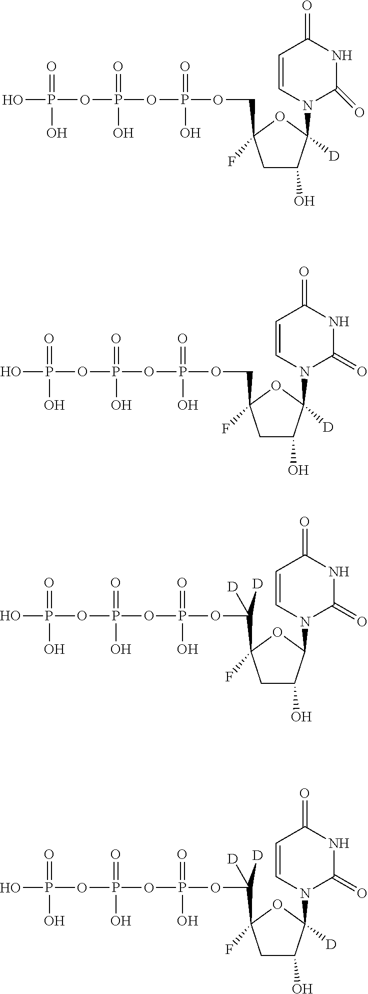

In exemplary embodiments, the compound is selected from:

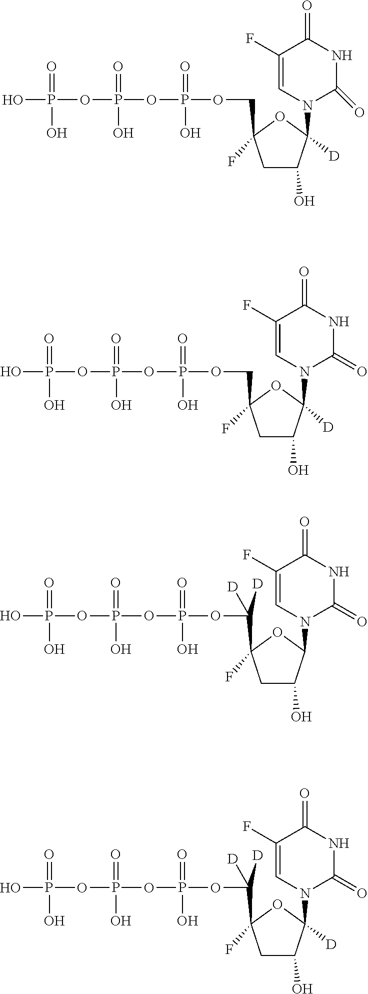

In exemplary embodiments, the compound is selected from:

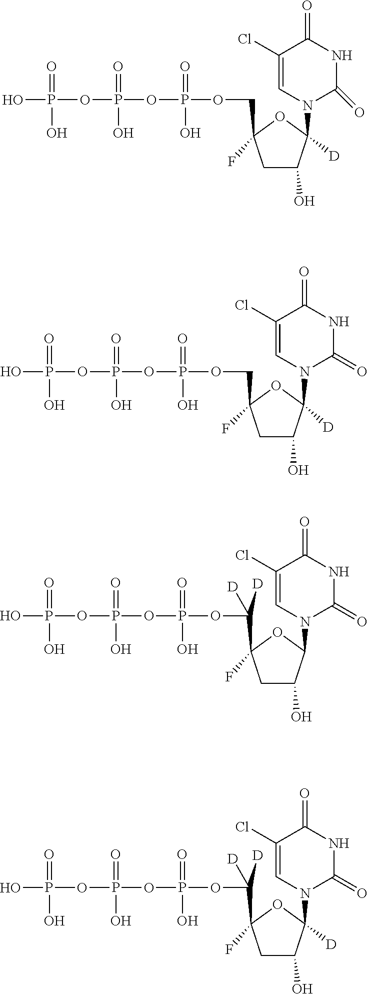

In exemplary embodiments, the compound is selected from:

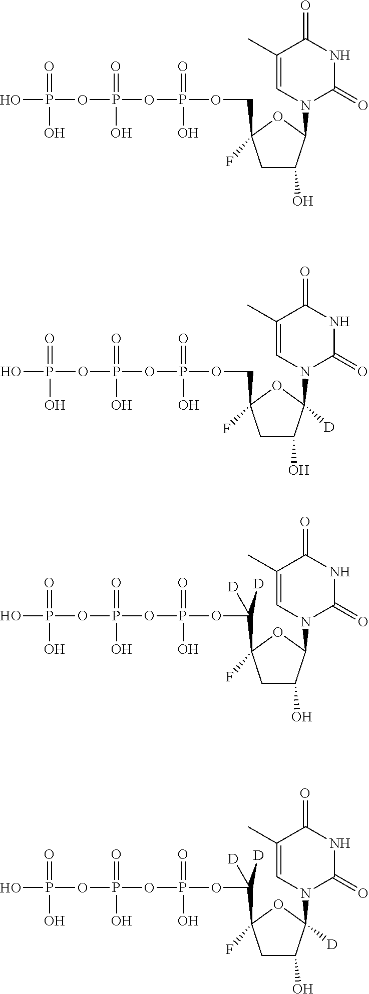

In exemplary embodiments, the compound is selected from:

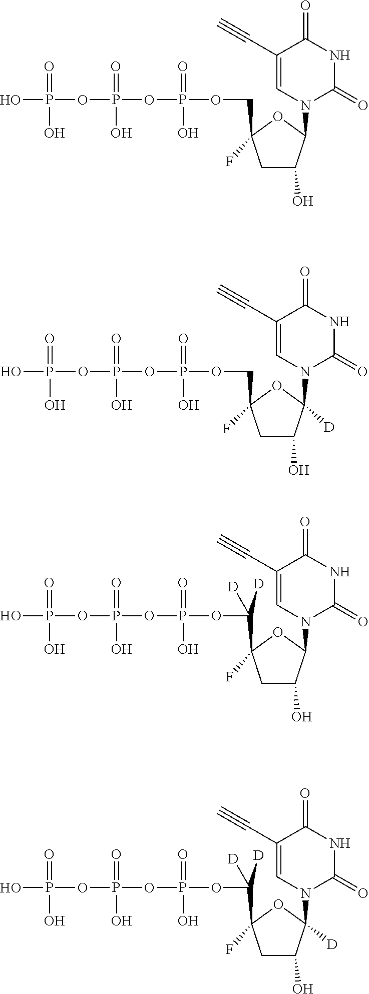

In exemplary embodiments, the compound is selected from:

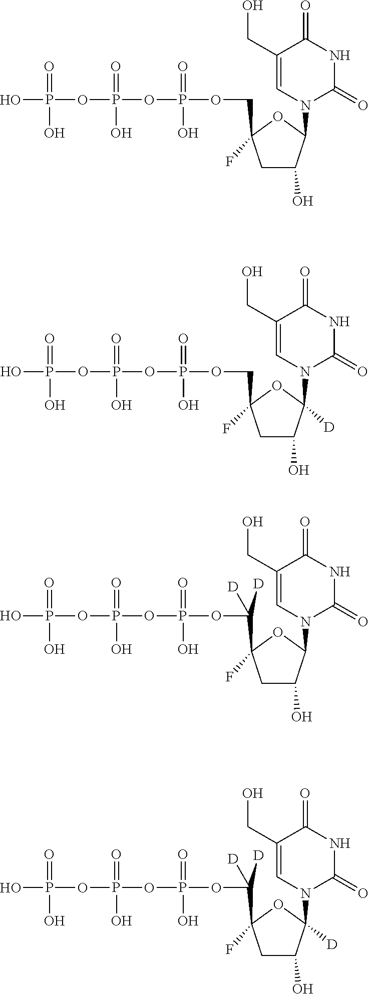

In exemplary embodiments, the compound is selected from:

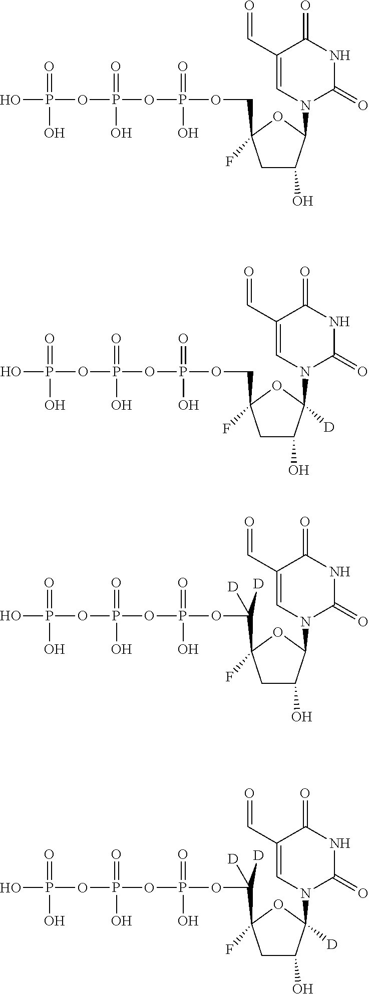

In exemplary embodiments, the compound is selected from:

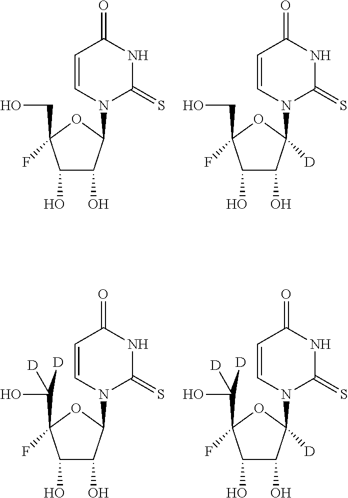

In exemplary embodiments, the compound is selected from:

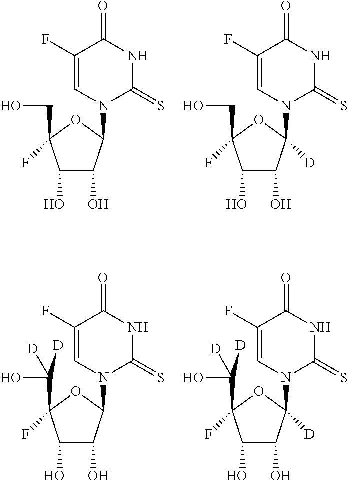

In exemplary embodiments, the compound is selected from:

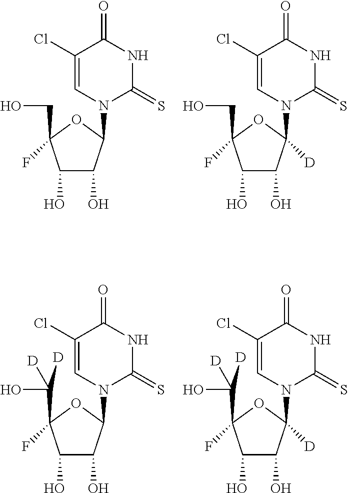

In exemplary embodiments, the compound is selected from:

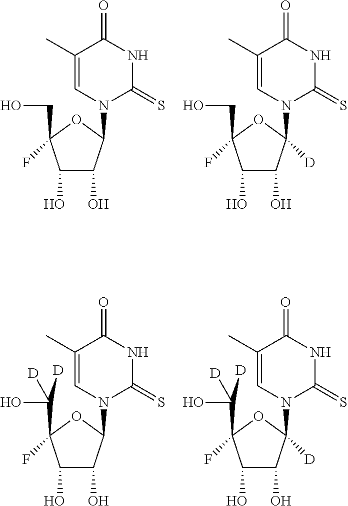

In exemplary embodiments, the compound is selected from:

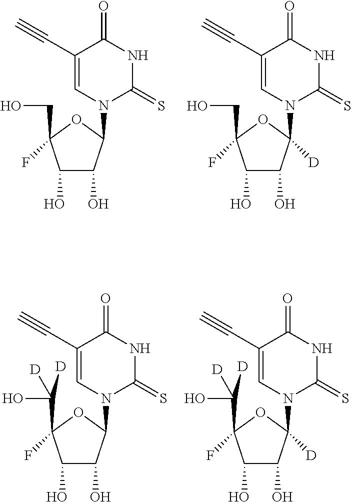

In exemplary embodiments, the compound is selected from:

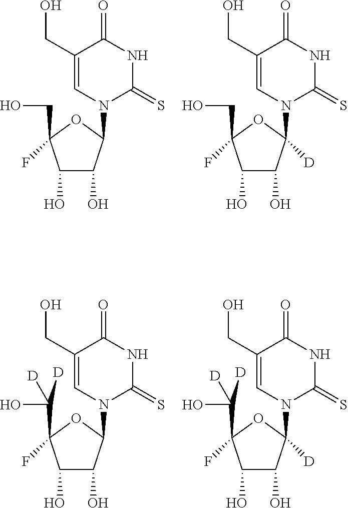

In exemplary embodiments, the compound is selected from:

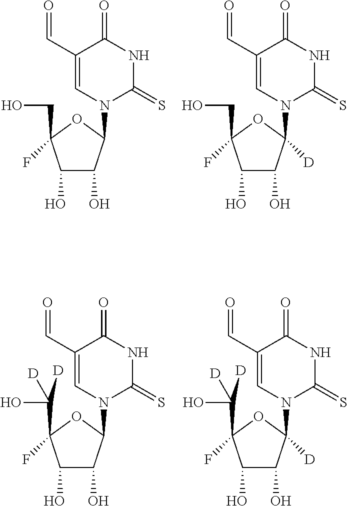

In exemplary embodiments, the compound is selected from:

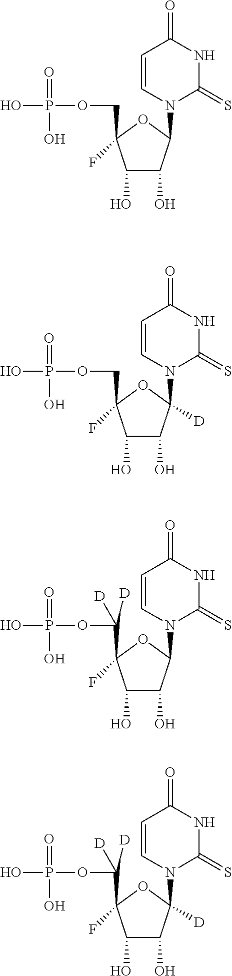 [SMILES: O=c1ccn([C@@H]2O[C@](F)(COP(=O)(O)O)[C@@H](O)[C@H]2O)c(=S)[nH]1 ; [2H][C@@]1(n2ccc(=O)[nH]c2=S)O[C@](F)(COP(=O)(O)O)[C@@H](O)[C@H]1O ; [2H]C([2H])(OP(=O)(O)O)[C@@]1(F)O[C@@H](n2ccc(=O)[nH]c2=S)[C@H](O)[C@@H]1O ; [2H]C([2H])(OP(=O)(O)O)[C@@]1(F)O[C@@]([2H])(n2ccc(=O)[nH]c2=S)[C@H](O)[C@@H]1O]

In exemplary embodiments, the compound is selected from:

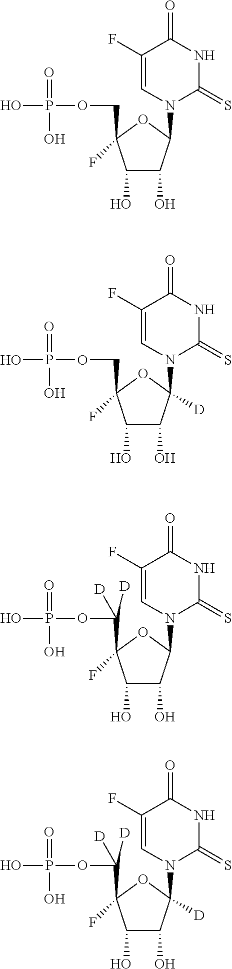 [SMILES: O=c1[nH]c(=S)n([C@@H]2O[C@](F)(COP(=O)(O)O)[C@@H](O)[C@H]2O)cc1F ; [2H][C@@]1(n2cc(F)c(=O)[nH]c2=S)O[C@](F)(COP(=O)(O)O)[C@@H](O)[C@H]1O ; [2H]C([2H])(OP(=O)(O)O)[C@@]1(F)O[C@@H](n2cc(F)c(=O)[nH]c2=S)[C@H](O)[C@@H]1O ; [2H]C([2H])(OP(=O)(O)O)[C@@]1(F)O[C@@]([2H])(n2cc(F)c(=O)[nH]c2=S)[C@H](O)[C@@H]1O]

In exemplary embodiments, the compound is selected from:

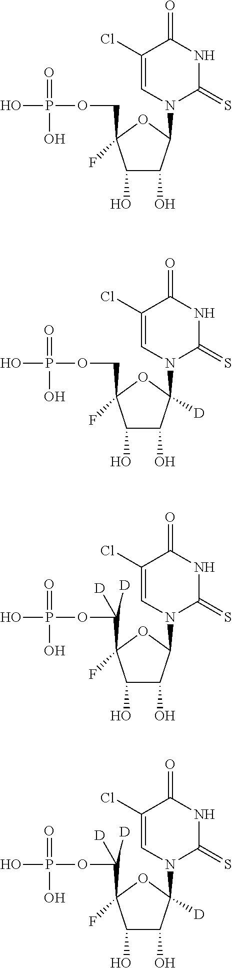 [SMILES: O=c1[nH]c(=S)n([C@@H]2O[C@](F)(COP(=O)(O)O)[C@@H](O)[C@H]2O)cc1Cl ; [2H][C@@]1(n2cc(Cl)c(=O)[nH]c2=S)O[C@](F)(COP(=O)(O)O)[C@@H](O)[C@H]1O ; [2H]C([2H])(OP(=O)(O)O)[C@@]1(F)O[C@@H](n2cc(Cl)c(=O)[nH]c2=S)[C@H](O)[C@@H]1O ; [2H]C([2H])(OP(=O)(O)O)[C@@]1(F)O[C@@]([2H])(n2cc(Cl)c(=O)[nH]c2=S)[C@H](O)[C@@H]1O]

In exemplary embodiments, the compound is selected from:

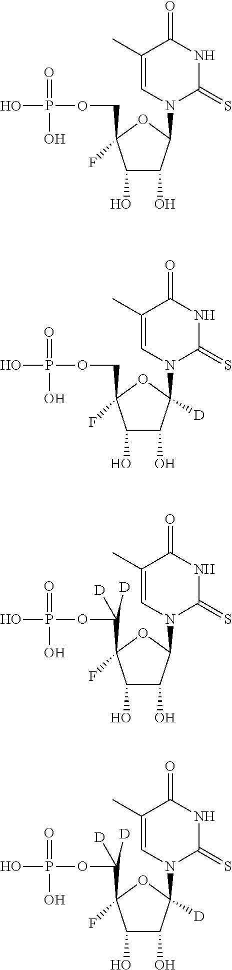 [SMILES: Cc1cn([C@@H]2O[C@](F)(COP(=O)(O)O)[C@@H](O)[C@H]2O)c(=S)[nH]c1=O ; [2H][C@@]1(n2cc(C)c(=O)[nH]c2=S)O[C@](F)(COP(=O)(O)O)[C@@H](O)[C@H]1O ; [2H]C([2H])(OP(=O)(O)O)[C@@]1(F)O[C@@H](n2cc(C)c(=O)[nH]c2=S)[C@H](O)[C@@H]1O ; [2H]C([2H])(OP(=O)(O)O)[C@@]1(F)O[C@@]([2H])(n2cc(C)c(=O)[nH]c2=S)[C@H](O)[C@@H]1O]

In exemplary embodiments, the compound is selected from:

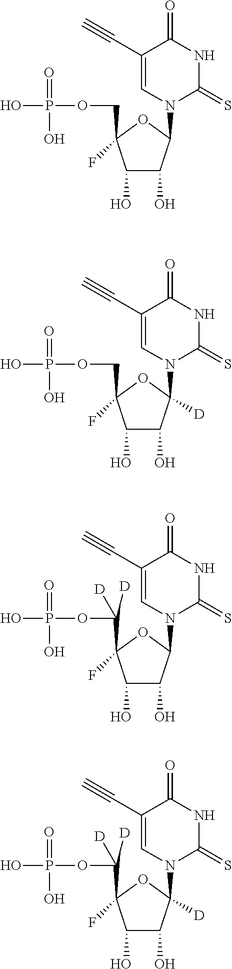 [SMILES: C#Cc1cn([C@@H]2O[C@](F)(COP(=O)(O)O)[C@@H](O)[C@H]2O)c(=S)[nH]c1=O ; [2H][C@@]1(n2cc(C#C)c(=O)[nH]c2=S)O[C@](F)(COP(=O)(O)O)[C@@H](O)[C@H]1O ; [2H]C([2H])(OP(=O)(O)O)[C@@]1(F)O[C@@H](n2cc(C#C)c(=O)[nH]c2=S)[C@H](O)[C@@H]1O ; [2H]C([2H])(OP(=O)(O)O)[C@@]1(F)O[C@@]([2H])(n2cc(C#C)c(=O)[nH]c2=S)[C@H](O)[C@@H]1O]

In exemplary embodiments, the compound is selected from:

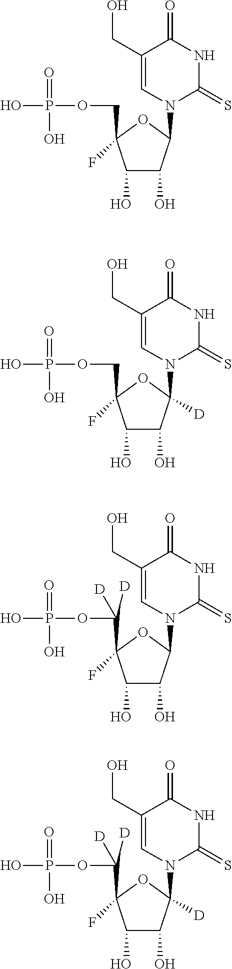 [SMILES: O=c1[nH]c(=S)n([C@@H]2O[C@](F)(COP(=O)(O)O)[C@@H](O)[C@H]2O)cc1CO ; [2H][C@@]1(n2cc(CO)c(=O)[nH]c2=S)O[C@](F)(COP(=O)(O)O)[C@@H](O)[C@H]1O ; [2H]C([2H])(OP(=O)(O)O)[C@@]1(F)O[C@@H](n2cc(CO)c(=O)[nH]c2=S)[C@H](O)[C@@H]1O ; [2H]C([2H])(OP(=O)(O)O)[C@@]1(F)O[C@@]([2H])(n2cc(CO)c(=O)[nH]c2=S)[C@H](O)[C@@H]1O]

In exemplary embodiments, the compound is selected from:

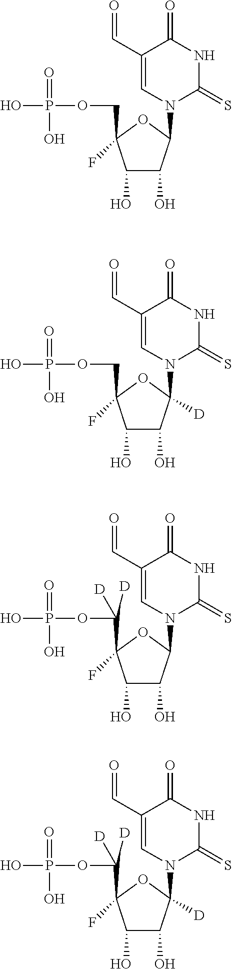 [SMILES: O=Cc1cn([C@@H]2O[C@](F)(COP(=O)(O)O)[C@@H](O)[C@H]2O)c(=S)[nH]c1=O ; [2H][C@@]1(n2cc(C=O)c(=O)[nH]c2=S)O[C@](F)(COP(=O)(O)O)[C@@H](O)[C@H]1O ; [2H]C([2H])(OP(=O)(O)O)[C@@]1(F)O[C@@H](n2cc(C=O)c(=O)[nH]c2=S)[C@H](O)[C@@H]1O ; [2H]C([2H])(OP(=O)(O)O)[C@@]1(F)O[C@@]([2H])(n2cc(C=O)c(=O)[nH]c2=S)[C@H](O)[C@@H]1O]

In exemplary embodiments, the compound is selected from:

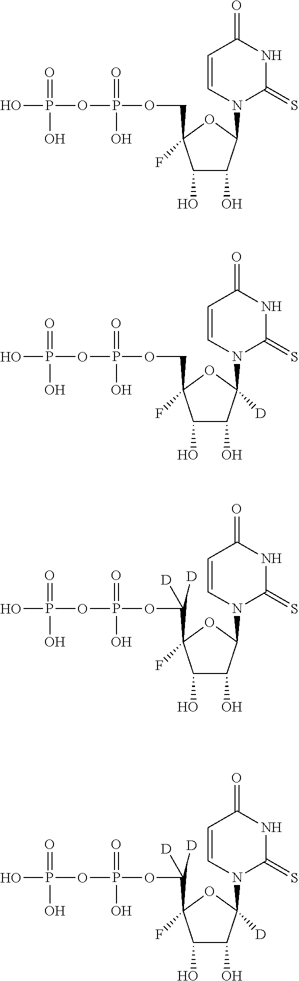 [SMILES: O=c1ccn([C@@H]2O[C@](F)(COP(=O)(O)OP(=O)(O)O)[C@@H](O)[C@H]2O)c(=S)[nH]1 ; [2H][C@@]1(n2ccc(=O)[nH]c2=S)O[C@](F)(COP(=O)(O)OP(=O)(O)O)[C@@H](O)[C@H]1O ; [2H]C([2H])(OP(=O)(O)OP(=O)(O)O)[C@@]1(F)O[C@@H](n2ccc(=O)[nH]c2=S)[C@H](O)[C@@H]1O ; [2H]C([2H])(OP(=O)(O)OP(=O)(O)O)[C@@]1(F)O[C@@]([2H])(n2ccc(=O)[nH]c2=S)[C@H](O)[C@@H]1O]

In exemplary embodiments, the compound is selected from:

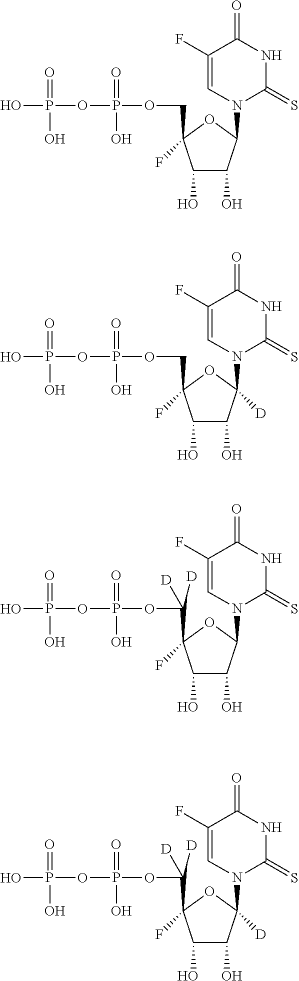 [SMILES: O=c1[nH]c(=S)n([C@@H]2O[C@](F)(COP(=O)(O)OP(=O)(O)O)[C@@H](O)[C@H]2O)cc1F ; [2H][C@@]1(n2cc(F)c(=O)[nH]c2=S)O[C@](F)(COP(=O)(O)OP(=O)(O)O)[C@@H](O)[C@H]1O ; [2H]C([2H])(OP(=O)(O)OP(=O)(O)O)[C@@]1(F)O[C@@H](n2cc(F)c(=O)[nH]c2=S)[C@H](O)[C@@H]1O ; [2H]C([2H])(OP(=O)(O)OP(=O)(O)O)[C@@]1(F)O[C@@]([2H])(n2cc(F)c(=O)[nH]c2=S)[C@H](O)[C@@H]1O]

In exemplary embodiments, the compound is selected from:

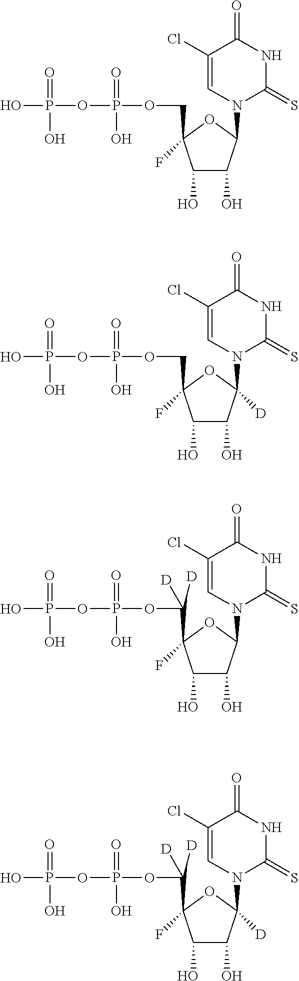 [SMILES: O=c1[nH]c(=S)n([C@@H]2O[C@](F)(COP(=O)(O)OP(=O)(O)O)[C@@H](O)[C@H]2O)cc1Cl ; [2H][C@@]1(n2cc(Cl)c(=O)[nH]c2=S)O[C@](F)(COP(=O)(O)OP(=O)(O)O)[C@@H](O)[C@H]1O ; [2H]C([2H])(OP(=O)(O)OP(=O)(O)O)[C@@]1(F)O[C@@H](n2cc(Cl)c(=O)[nH]c2=S)[C@H](O)[C@@H]1O ; [2H]C([2H])(OP(=O)(O)OP(=O)(O)O)[C@@]1(F)O[C@@]([2H])(n2cc(Cl)c(=O)[nH]c2=S)[C@H](O)[C@@H]1O]

In exemplary embodiments, the compound is selected from:

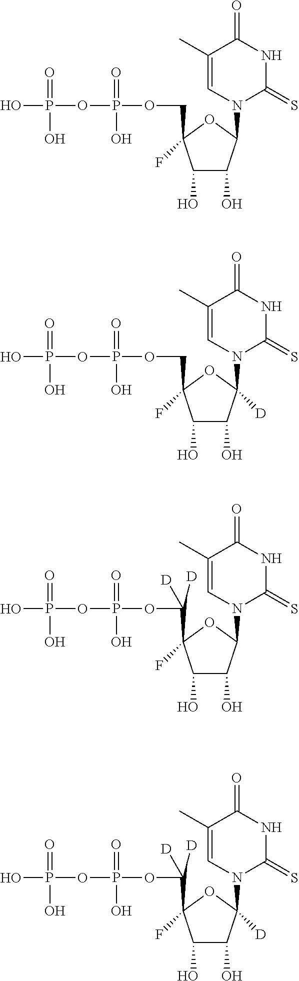 [SMILES: Cc1cn([C@@H]2O[C@](F)(COP(=O)(O)OP(=O)(O)O)[C@@H](O)[C@H]2O)c(=S)[nH]c1=O ; [2H][C@@]1(n2cc(C)c(=O)[nH]c2=S)O[C@](F)(COP(=O)(O)OP(=O)(O)O)[C@@H](O)[C@H]1O ; [2H]C([2H])(OP(=O)(O)OP(=O)(O)O)[C@@]1(F)O[C@@H](n2cc(C)c(=O)[nH]c2=S)[C@H](O)[C@@H]1O ; [2H]C([2H])(OP(=O)(O)OP(=O)(O)O)[C@@]1(F)O[C@@]([2H])(n2cc(C)c(=O)[nH]c2=S)[C@H](O)[C@@H]1O]

In exemplary embodiments, the compound is selected from:

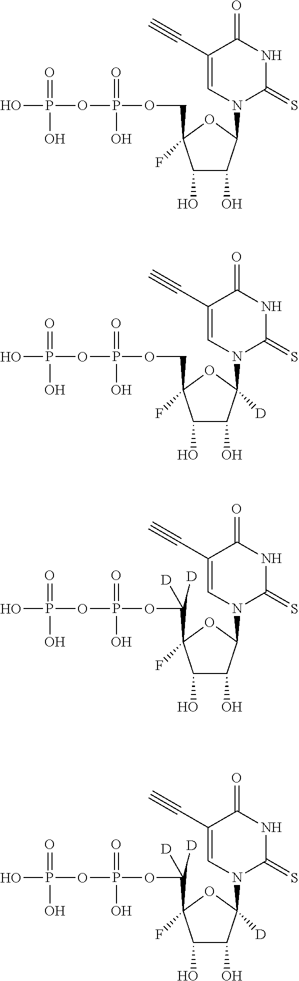 [SMILES: C#Cc1cn([C@@H]2O[C@](F)(COP(=O)(O)OP(=O)(O)O)[C@@H](O)[C@H]2O)c(=S)[nH]c1=O ; [2H][C@@]1(n2cc(C#C)c(=O)[nH]c2=S)O[C@](F)(COP(=O)(O)OP(=O)(O)O)[C@@H](O)[C@H]1O ; [2H]C([2H])(OP(=O)(O)OP(=O)(O)O)[C@@]1(F)O[C@@H](n2cc(C#C)c(=O)[nH]c2=S)[C@H](O)[C@@H]1O ; [2H]C([2H])(OP(=O)(O)OP(=O)(O)O)[C@@]1(F)O[C@@]([2H])(n2cc(C#C)c(=O)[nH]c2=S)[C@H](O)[C@@H]1O]

In exemplary embodiments, the compound is selected from:

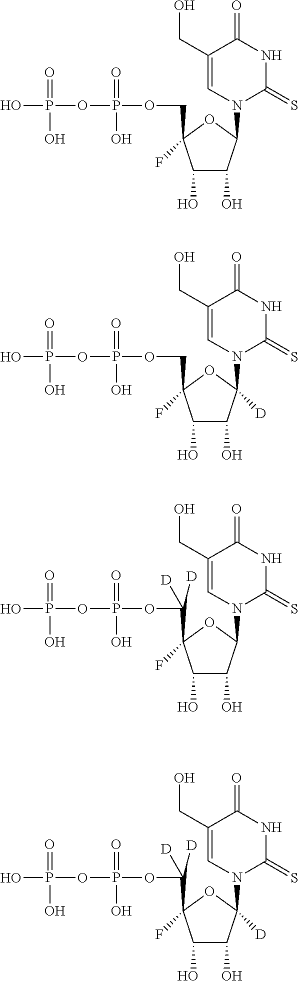 [SMILES: O=c1[nH]c(=S)n([C@@H]2O[C@](F)(COP(=O)(O)OP(=O)(O)O)[C@@H](O)[C@H]2O)cc1CO ; [2H][C@@]1(n2cc(CO)c(=O)[nH]c2=S)O[C@](F)(COP(=O)(O)OP(=O)(O)O)[C@@H](O)[C@H]1O ; [2H]C([2H])(OP(=O)(O)OP(=O)(O)O)[C@@]1(F)O[C@@H](n2cc(CO)c(=O)[nH]c2=S)[C@H](O)[C@@H]1O ; [2H]C([2H])(OP(=O)(O)OP(=O)(O)O)[C@@]1(F)O[C@@]([2H])(n2cc(CO)c(=O)[nH]c2=S)[C@H](O)[C@@H]1O]

In exemplary embodiments, the compound is selected from:

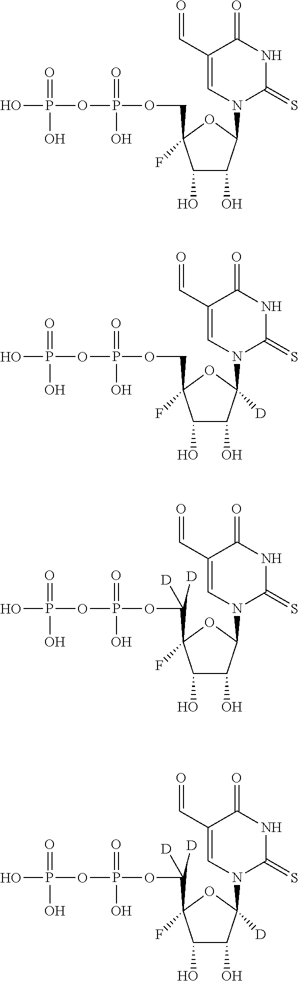 [SMILES: O=Cc1cn([C@@H]2O[C@](F)(COP(=O)(O)OP(=O)(O)O)[C@@H](O)[C@H]2O)c(=S)[nH]c1=O ; [2H][C@@]1(n2cc(C=O)c(=O)[nH]c2=S)O[C@](F)(COP(=O)(O)OP(=O)(O)O)[C@@H](O)[C@H]1O ; [2H]C([2H])(OP(=O)(O)OP(=O)(O)O)[C@@]1(F)O[C@@H](n2cc(C=O)c(=O)[nH]c2=S)[C@H](O)[C@@H]1O ; [2H]C([2H])(OP(=O)(O)OP(=O)(O)O)[C@@]1(F)O[C@@]([2H])(n2cc(C=O)c(=O)[nH]c2=S)[C@H](O)[C@@H]1O]

In exemplary embodiments, the compound is selected from:

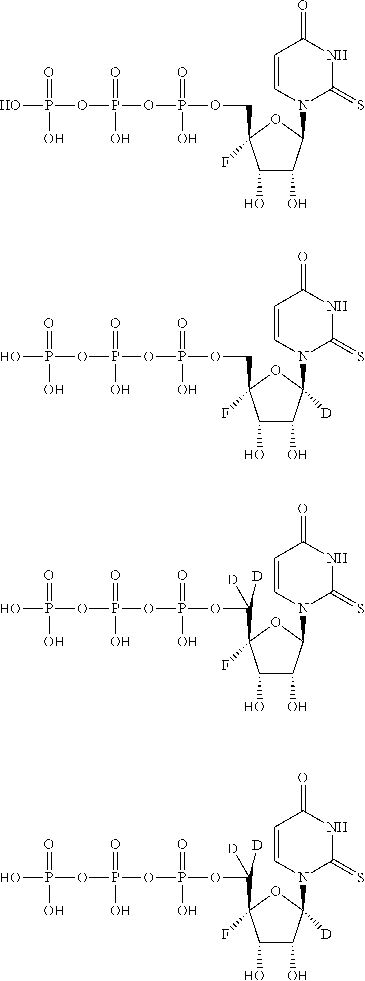 [SMILES: O=c1ccn([C@@H]2O[C@](F)(COP(=O)(O)OP(=O)(O)OP(=O)(O)O)[C@@H](O)[C@H]2O)c(=S)[nH]1 ; [2H][C@@]1(n2ccc(=O)[nH]c2=S)O[C@](F)(COP(=O)(O)OP(=O)(O)OP(=O)(O)O)[C@@H](O)[C@H]1O ; [2H]C([2H])(OP(=O)(O)OP(=O)(O)OP(=O)(O)O)[C@@]1(F)O[C@@H](n2ccc(=O)[nH]c2=S)[C@H](O)[C@@H]1O ; [2H]C([2H])(OP(=O)(O)OP(=O)(O)OP(=O)(O)O)[C@@]1(F)O[C@@]([2H])(n2ccc(=O)[nH]c2=S)[C@H](O)[C@@H]1O]

In exemplary embodiments, the compound is selected from:

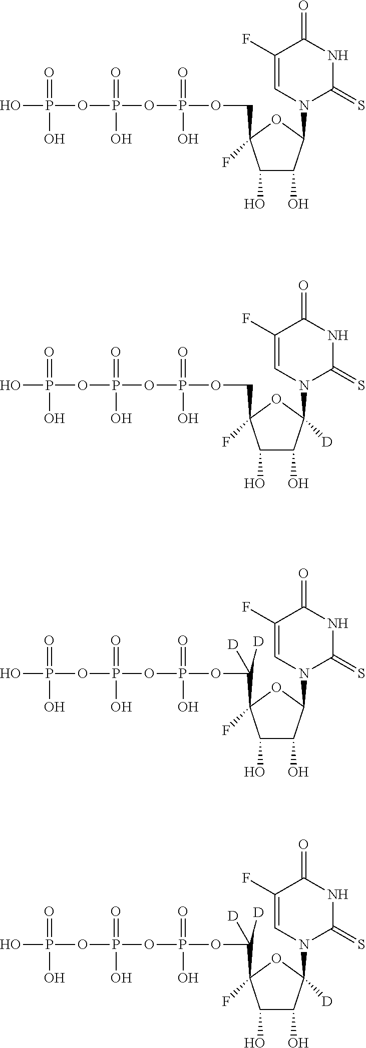 [SMILES: O=c1[nH]c(=S)n([C@@H]2O[C@](F)(COP(=O)(O)OP(=O)(O)OP(=O)(O)O)[C@@H](O)[C@H]2O)cc1F ; [2H][C@@]1(n2cc(F)c(=O)[nH]c2=S)O[C@](F)(COP(=O)(O)OP(=O)(O)OP(=O)(O)O)[C@@H](O)[C@H]1O ; [2H]C([2H])(OP(=O)(O)OP(=O)(O)OP(=O)(O)O)[C@@]1(F)O[C@@H](n2cc(F)c(=O)[nH]c2=S)[C@H](O)[C@@H]1O ; [2H]C([2H])(OP(=O)(O)OP(=O)(O)OP(=O)(O)O)[C@@]1(F)O[C@@]([2H])(n2cc(F)c(=O)[nH]c2=S)[C@H](O)[C@@H]1O]

In exemplary embodiments, the compound is selected from:

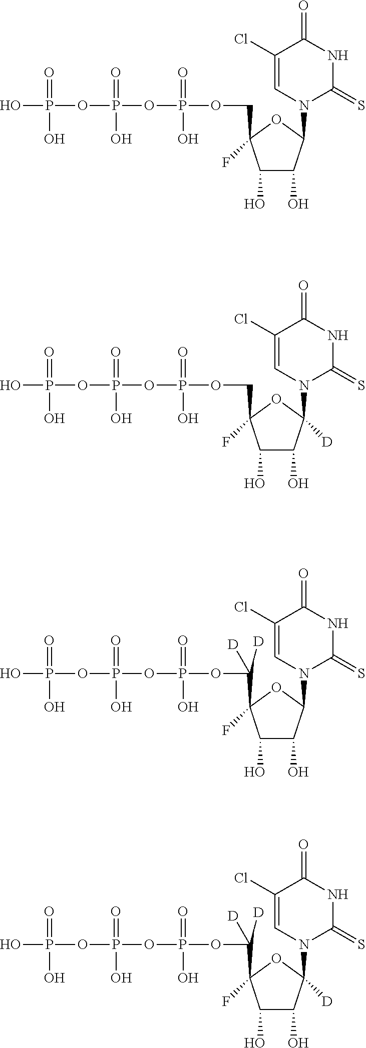 [SMILES: O=c1[nH]c(=S)n([C@@H]2O[C@](F)(COP(=O)(O)OP(=O)(O)OP(=O)(O)O)[C@@H](O)[C@H]2O)cc1Cl ; [2H][C@@]1(n2cc(Cl)c(=O)[nH]c2=S)O[C@](F)(COP(=O)(O)OP(=O)(O)OP(=O)(O)O)[C@@H](O)[C@H]1O ; [2H]C([2H])(OP(=O)(O)OP(=O)(O)OP(=O)(O)O)[C@@]1(F)O[C@@H](n2cc(Cl)c(=O)[nH]c2=S)[C@H](O)[C@@H]1O ; [2H]C([2H])(OP(=O)(O)OP(=O)(O)OP(=O)(O)O)[C@@]1(F)O[C@@]([2H])(n2cc(Cl)c(=O)[nH]c2=S)[C@H](O)[C@@H]1O]

In exemplary embodiments, the compound is selected from:

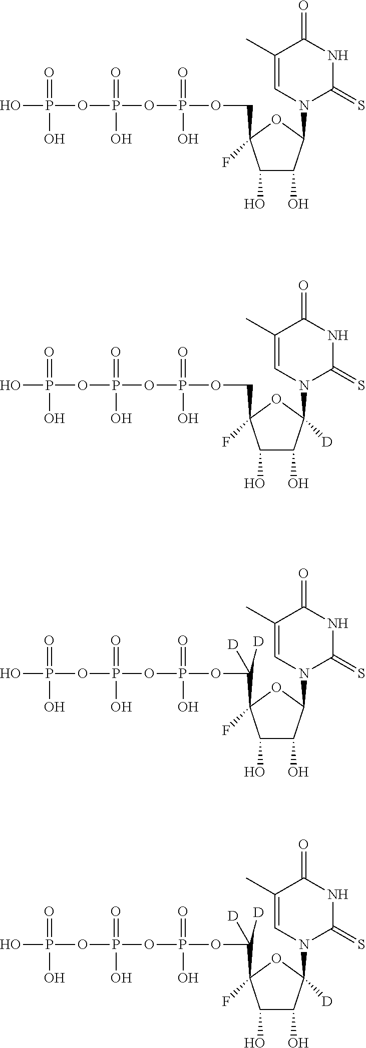 [SMILES: Cc1cn([C@@H]2O[C@](F)(COP(=O)(O)OP(=O)(O)OP(=O)(O)O)[C@@H](O)[C@H]2O)c(=S)[nH]c1=O ; [2H][C@@]1(n2cc(C)c(=O)[nH]c2=S)O[C@](F)(COP(=O)(O)OP(=O)(O)OP(=O)(O)O)[C@@H](O)[C@H]1O ; [2H]C([2H])(OP(=O)(O)OP(=O)(O)OP(=O)(O)O)[C@@]1(F)O[C@@H](n2cc(C)c(=O)[nH]c2=S)[C@H](O)[C@@H]1O ; [2H]C([2H])(OP(=O)(O)OP(=O)(O)OP(=O)(O)O)[C@@]1(F)O[C@@]([2H])(n2cc(C)c(=O)[nH]c2=S)[C@H](O)[C@@H]1O]

In exemplary embodiments, the compound is selected from:

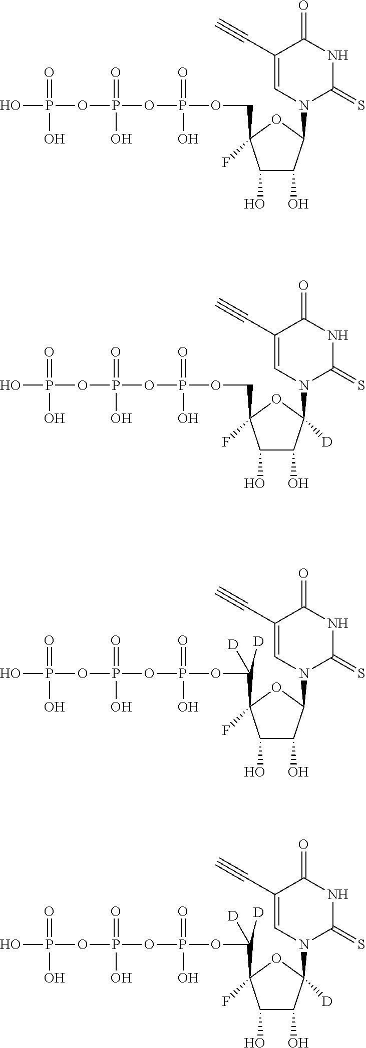 [SMILES: C#Cc1cn([C@@H]2O[C@](F)(COP(=O)(O)OP(=O)(O)OP(=O)(O)O)[C@@H](O)[C@H]2O)c(=S)[nH]c1=O ; [2H][C@@]1(n2cc(C#C)c(=O)[nH]c2=S)O[C@](F)(COP(=O)(O)OP(=O)(O)OP(=O)(O)O)[C@@H](O)[C@H]1O ; [2H]C([2H])(OP(=O)(O)OP(=O)(O)OP(=O)(O)O)[C@@]1(F)O[C@@H](n2cc(C#C)c(=O)[nH]c2=S)[C@H](O)[C@@H]1O ; [2H]C([2H])(OP(=O)(O)OP(=O)(O)OP(=O)(O)O)[C@@]1(F)O[C@@]([2H])(n2cc(C#C)c(=O)[nH]c2=S)[C@H](O)[C@@H]1O]

In exemplary embodiments, the compound is selected from:

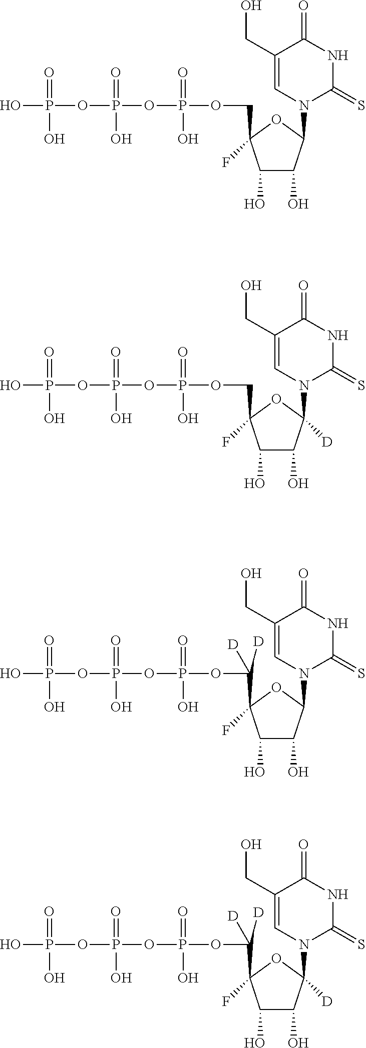 [SMILES: O=c1[nH]c(=S)n([C@@H]2O[C@](F)(COP(=O)(O)OP(=O)(O)OP(=O)(O)O)[C@@H](O)[C@H]2O)cc1CO ; [2H][C@@]1(n2cc(CO)c(=O)[nH]c2=S)O[C@](F)(COP(=O)(O)OP(=O)(O)OP(=O)(O)O)[C@@H](O)[C@H]1O ; [2H]C([2H])(OP(=O)(O)OP(=O)(O)OP(=O)(O)O)[C@@]1(F)O[C@@H](n2cc(CO)c(=O)[nH]c2=S)[C@H](O)[C@@H]1O ; [2H]C([2H])(OP(=O)(O)OP(=O)(O)OP(=O)(O)O)[C@@]1(F)O[C@@]([2H])(n2cc(CO)c(=O)[nH]c2=S)[C@H](O)[C@@H]1O]

In exemplary embodiments, the compound is selected from:

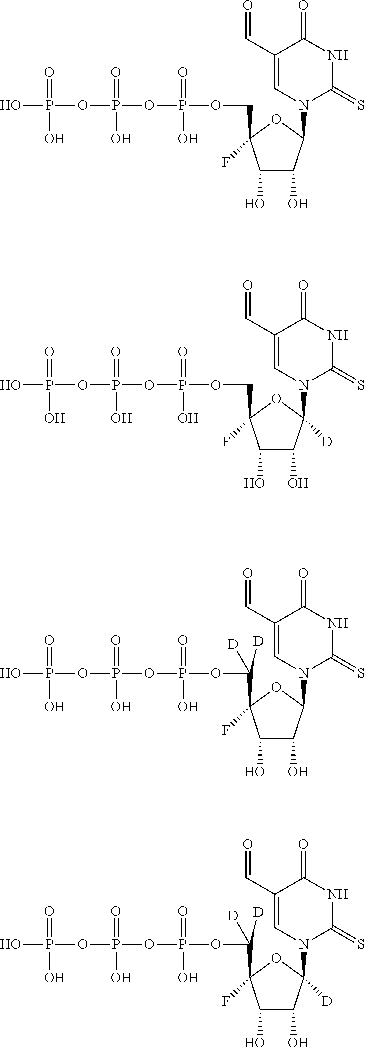 [SMILES: O=Cc1cn([C@@H]2O[C@](F)(COP(=O)(O)OP(=O)(O)OP(=O)(O)O)[C@@H](O)[C@H]2O)c(=S)[nH]c1=O ; [2H][C@@]1(n2cc(C=O)c(=O)[nH]c2=S)O[C@](F)(COP(=O)(O)OP(=O)(O)OP(=O)(O)O)[C@@H](O)[C@H]1O ; [2H]C([2H])(OP(=O)(O)OP(=O)(O)OP(=O)(O)O)[C@@]1(F)O[C@@H](n2cc(C=O)c(=O)[nH]c2=S)[C@H](O)[C@@H]1O ; [2H]C([2H])(OP(=O)(O)OP(=O)(O)OP(=O)(O)O)[C@@]1(F)O[C@@]([2H])(n2cc(C=O)c(=O)[nH]c2=S)[C@H](O)[C@@H]1O]

In exemplary embodiments, the compound is selected from:

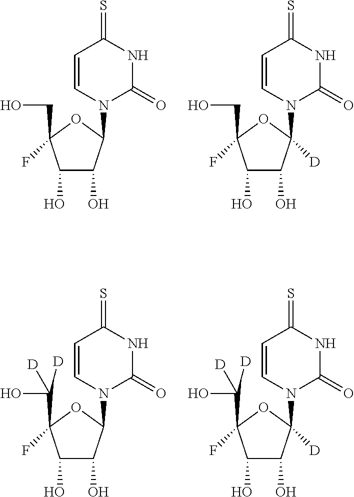

In exemplary embodiments, the compound is selected from:

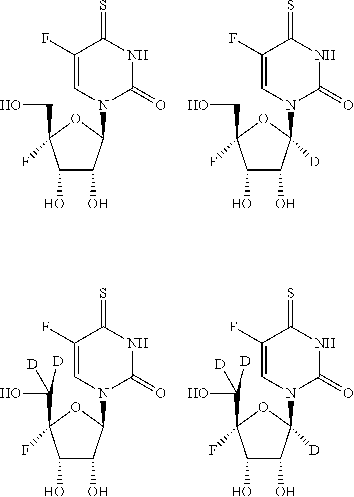

In exemplary embodiments, the compound is selected from:

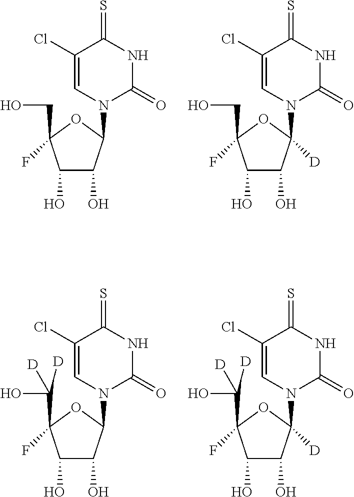

In exemplary embodiments, the compound is selected from:

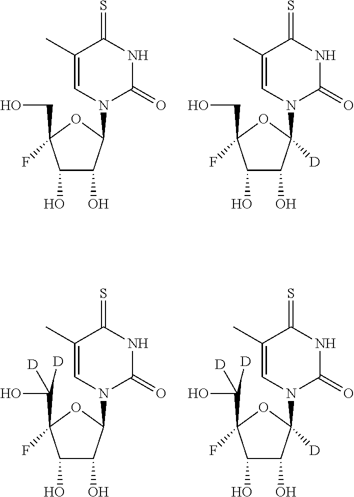

In exemplary embodiments, the compound is selected from:

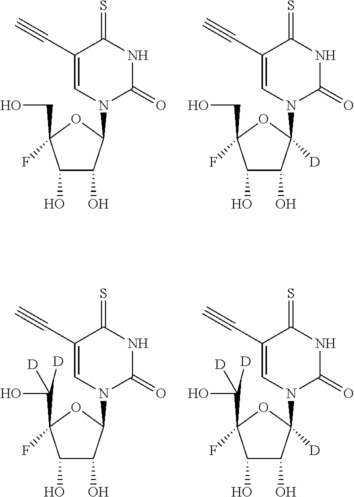

In exemplary embodiments, the compound is selected from:

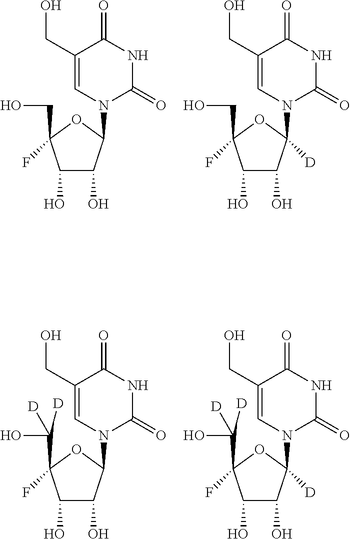

In exemplary embodiments, the compound is selected from:

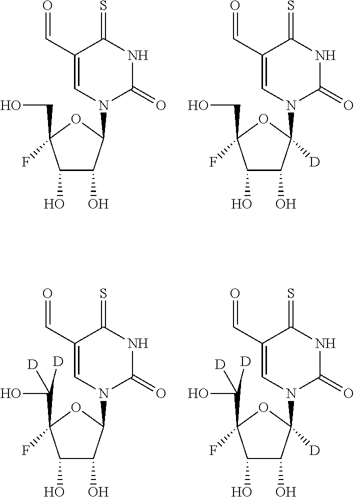

In exemplary embodiments, the compound is selected from:

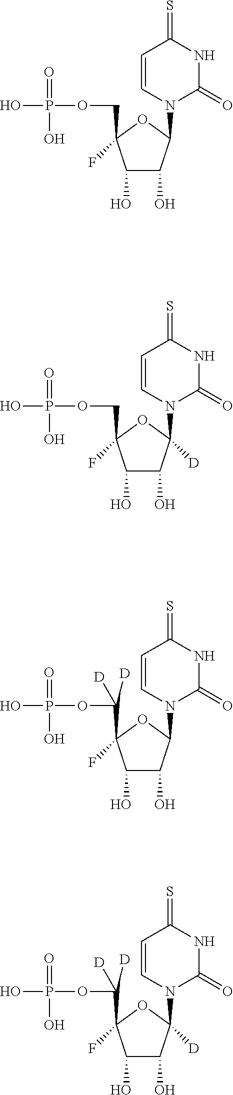 [SMILES: O=c1[nH]c(=S)ccn1[C@@H]1O[C@](F)(COP(=O)(O)O)[C@@H](O)[C@H]1O ; [2H][C@@]1(n2ccc(=S)[nH]c2=O)O[C@](F)(COP(=O)(O)O)[C@@H](O)[C@H]1O ; [2H]C([2H])(OP(=O)(O)O)[C@@]1(F)O[C@@H](n2ccc(=S)[nH]c2=O)[C@H](O)[C@@H]1O ; [2H]C([2H])(OP(=O)(O)O)[C@@]1(F)O[C@@]([2H])(n2ccc(=S)[nH]c2=O)[C@H](O)[C@@H]1O]

In exemplary embodiments, the compound is selected from:

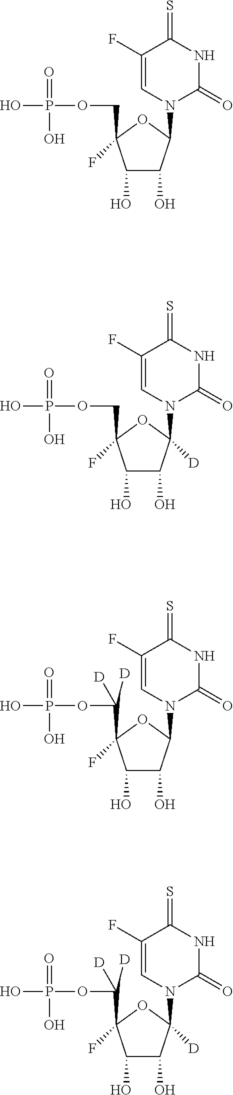 [SMILES: O=c1[nH]c(=S)c(F)cn1[C@@H]1O[C@](F)(COP(=O)(O)O)[C@@H](O)[C@H]1O ; [2H][C@@]1(n2cc(F)c(=S)[nH]c2=O)O[C@](F)(COP(=O)(O)O)[C@@H](O)[C@H]1O ; [2H]C([2H])(OP(=O)(O)O)[C@@]1(F)O[C@@H](n2cc(F)c(=S)[nH]c2=O)[C@H](O)[C@@H]1O ; [2H]C([2H])(OP(=O)(O)O)[C@@]1(F)O[C@@]([2H])(n2cc(F)c(=S)[nH]c2=O)[C@H](O)[C@@H]1O]

In exemplary embodiments, the compound is selected from:

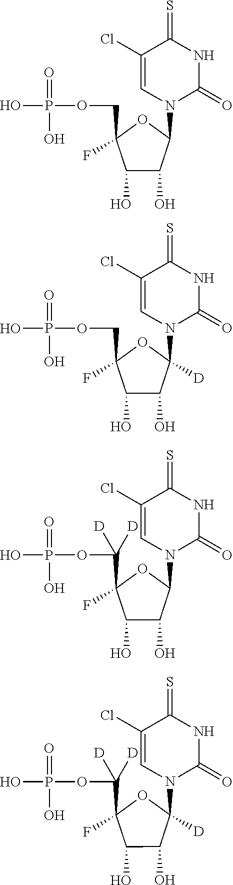

In exemplary embodiments, the compound is selected from:

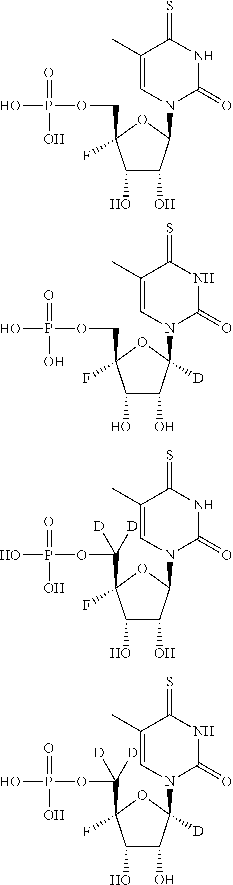

In exemplary embodiments, the compound is selected from:

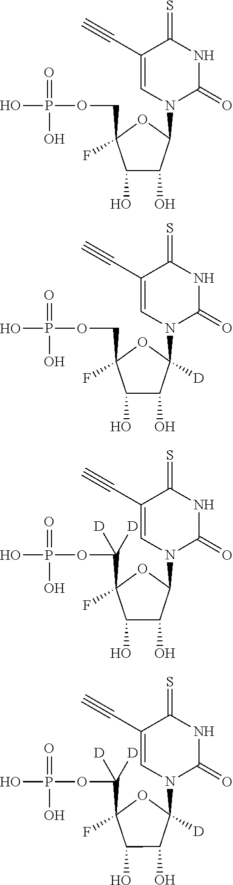

In exemplary embodiments, the compound is selected from:

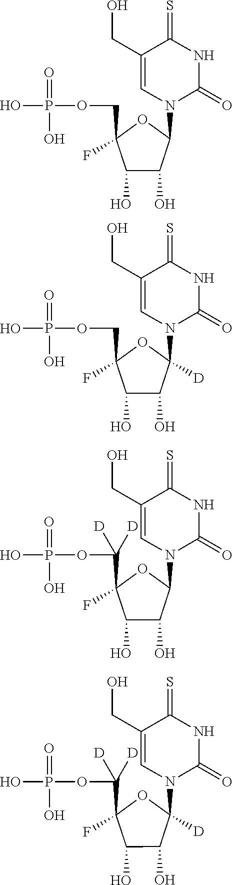

In exemplary embodiments, the compound is selected from:

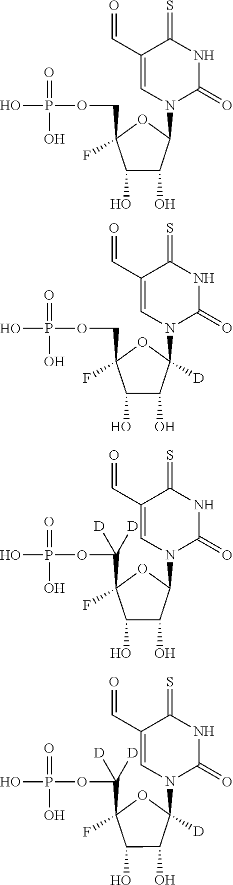

In exemplary embodiments, the compound is selected from:

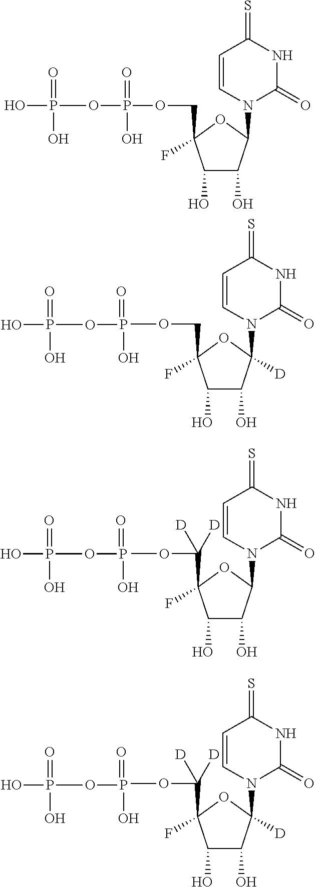

In exemplary embodiments, the compound is selected from:

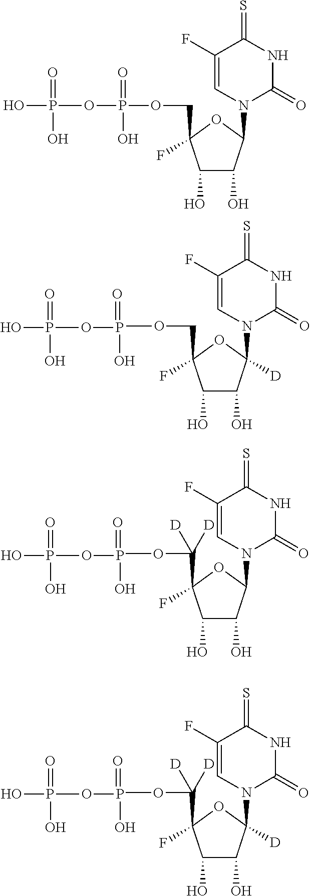

In exemplary embodiments, the compound is selected from:

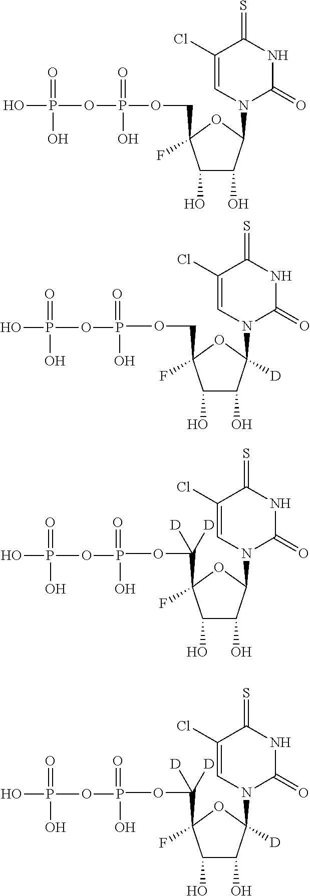

In exemplary embodiments, the compound is selected from:

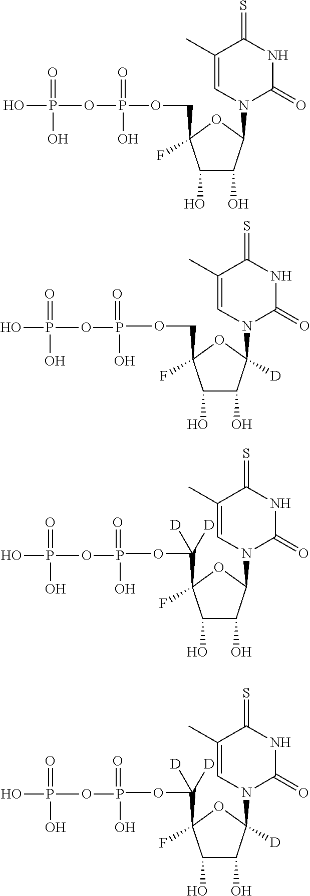

In exemplary embodiments, the compound is selected from:

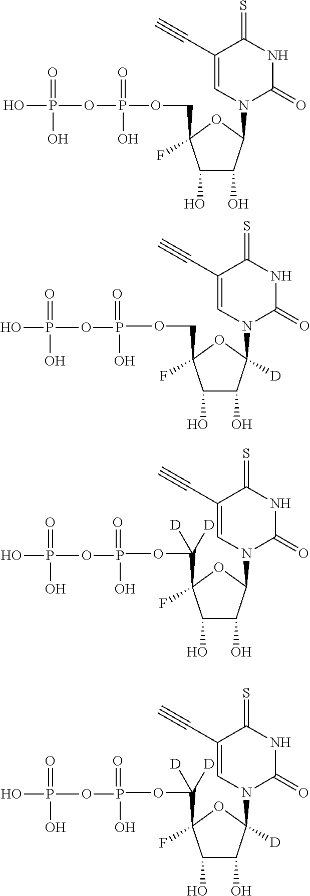

In exemplary embodiments, the compound is selected from:

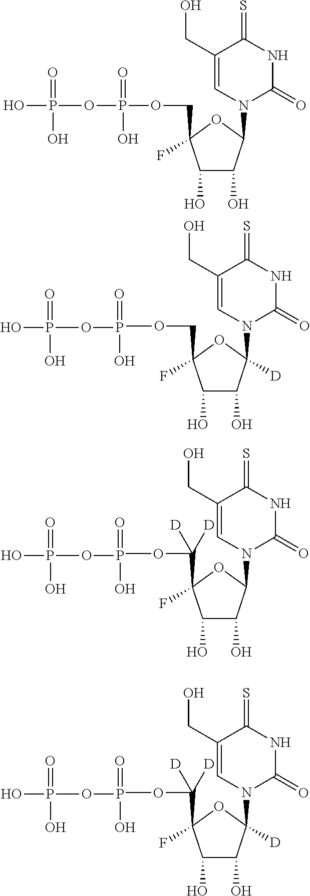

In exemplary embodiments, the compound is selected from:

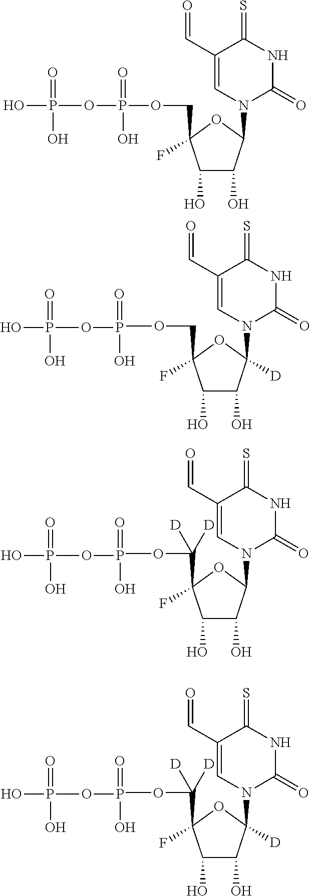

In exemplary embodiments, the compound is selected from:

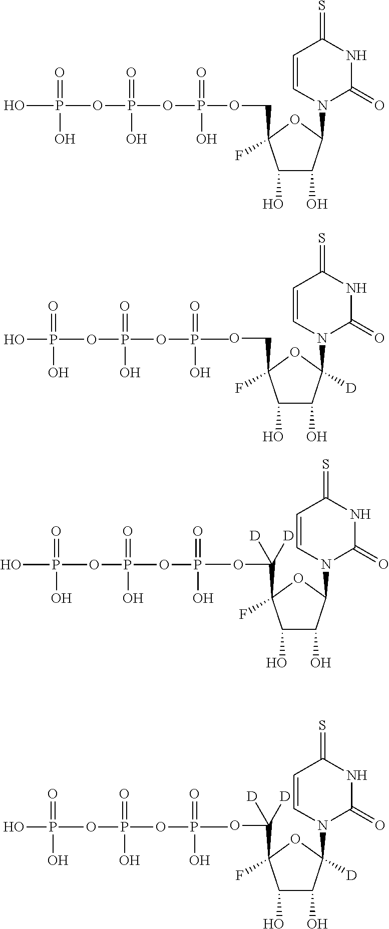

In exemplary embodiments, the compound is selected from:

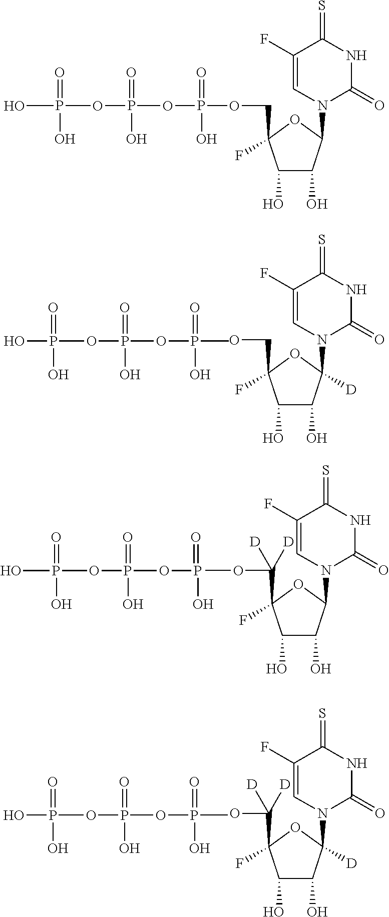

In exemplary embodiments, the compound is selected from:

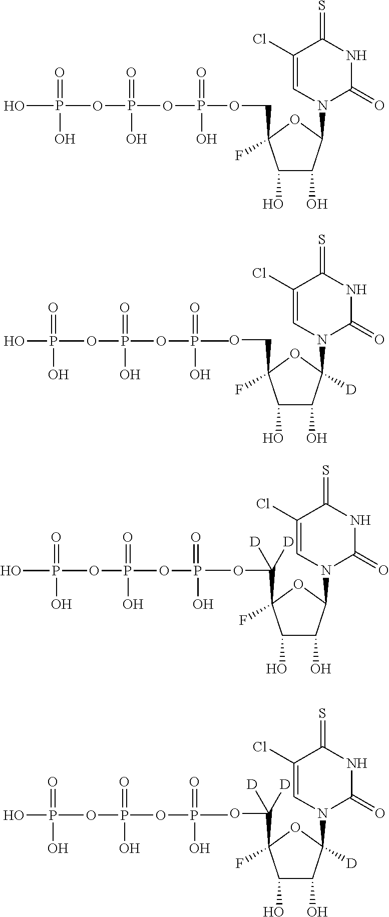

In exemplary embodiments, the compound is selected from:

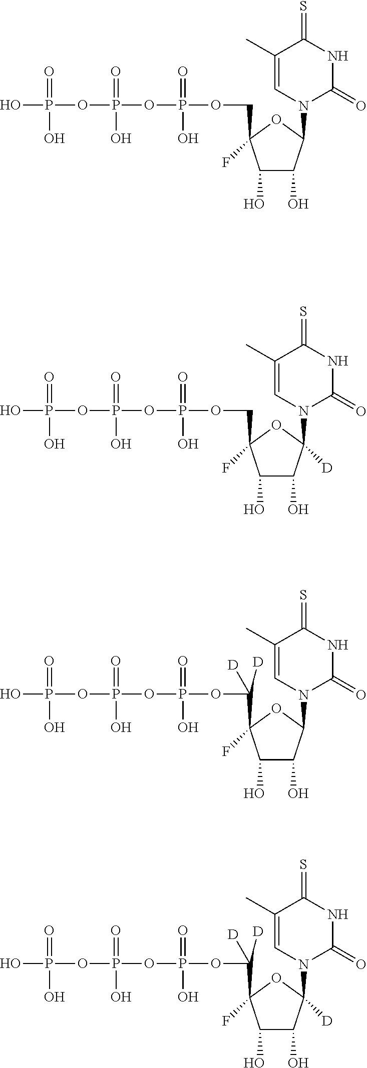 [SMILES: Cc1cn([C@@H]2O[C@](F)(COP(=O)(O)OP(=O)(O)OP(=O)(O)O)[C@@H](O)[C@H]2O)c(=O)[nH]c1=S ; [2H][C@@]1(n2cc(C)c(=S)[nH]c2=O)O[C@](F)(COP(=O)(O)OP(=O)(O)OP(=O)(O)O)[C@@H](O)[C@H]1O ; [2H]C([2H])(OP(=O)(O)OP(=O)(O)OP(=O)(O)O)[C@@]1(F)O[C@@H](n2cc(C)c(=S)[nH]c2=O)[C@H](O)[C@@H]1O ; [2H]C([2H])(OP(=O)(O)OP(=O)(O)OP(=O)(O)O)[C@@]1(F)O[C@@]([2H])(n2cc(C)c(=S)[nH]c2=O)[C@H](O)[C@@H]1O]

In exemplary embodiments, the compound is selected from:

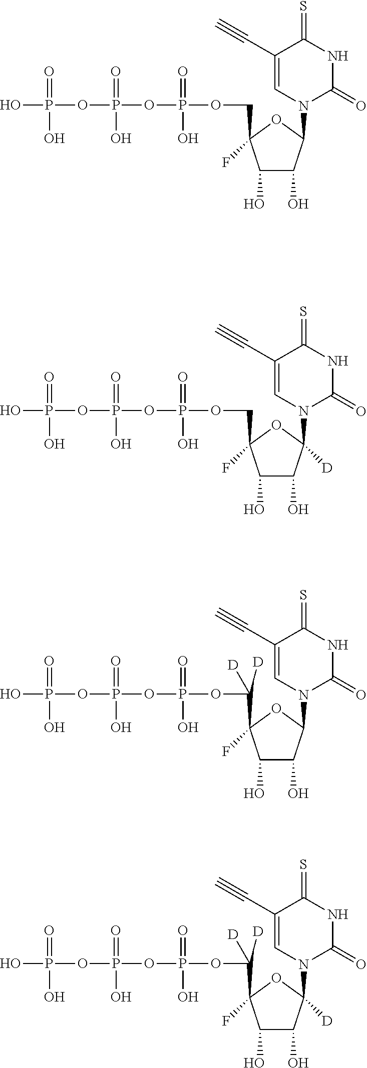 [SMILES: C#Cc1cn([C@@H]2O[C@](F)(COP(=O)(O)OP(=O)(O)OP(=O)(O)O)[C@@H](O)[C@H]2O)c(=O)[nH]c1=S ; [2H][C@@]1(n2cc(C#C)c(=S)[nH]c2=O)O[C@](F)(COP(=O)(O)OP(=O)(O)OP(=O)(O)O)[C@@H](O)[C@H]1O ; [2H]C([2H])(OP(=O)(O)OP(=O)(O)OP(=O)(O)O)[C@@]1(F)O[C@@H](n2cc(C#C)c(=S)[nH]c2=O)[C@H](O)[C@@H]1O ; [2H]C([2H])(OP(=O)(O)OP(=O)(O)OP(=O)(O)O)[C@@]1(F)O[C@@]([2H])(n2cc(C#C)c(=S)[nH]c2=O)[C@H](O)[C@@H]1O]

In exemplary embodiments, the compound is selected from:

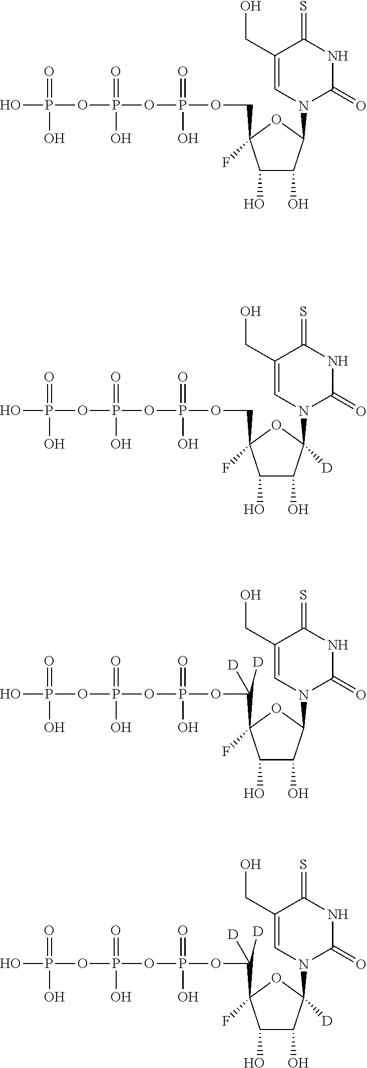 [SMILES: O=c1[nH]c(=S)c(CO)cn1[C@@H]1O[C@](F)(COP(=O)(O)OP(=O)(O)OP(=O)(O)O)[C@@H](O)[C@H]1O ; [2H][C@@]1(n2cc(CO)c(=S)[nH]c2=O)O[C@](F)(COP(=O)(O)OP(=O)(O)OP(=O)(O)O)[C@@H](O)[C@H]1O ; [2H]C([2H])(OP(=O)(O)OP(=O)(O)OP(=O)(O)O)[C@@]1(F)O[C@@H](n2cc(CO)c(=S)[nH]c2=O)[C@H](O)[C@@H]1O ; [2H]C([2H])(OP(=O)(O)OP(=O)(O)OP(=O)(O)O)[C@@]1(F)O[C@@]([2H])(n2cc(CO)c(=S)[nH]c2=O)[C@H](O)[C@@H]1O]

In exemplary embodiments, the compound is selected from:

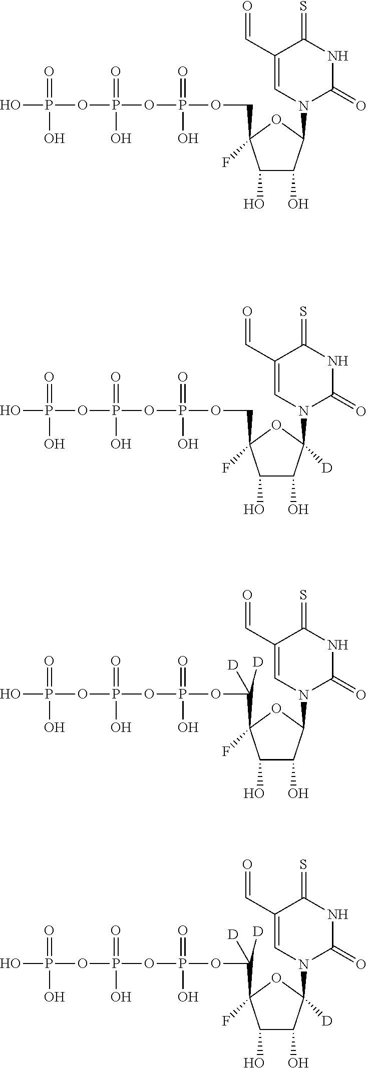 [SMILES: O=Cc1cn([C@@H]2O[C@](F)(COP(=O)(O)OP(=O)(O)OP(=O)(O)O)[C@@H](O)[C@H]2O)c(=O)[nH]c1=S ; [2H][C@@]1(n2cc(C=O)c(=S)[nH]c2=O)O[C@](F)(COP(=O)(O)OP(=O)(O)OP(=O)(O)O)[C@@H](O)[C@H]1O ; [2H]C([2H])(OP(=O)(O)OP(=O)(O)OP(=O)(O)O)[C@@]1(F)O[C@@H](n2cc(C=O)c(=S)[nH]c2=O)[C@H](O)[C@@H]1O ; [2H]C([2H])(OP(=O)(O)OP(=O)(O)OP(=O)(O)O)[C@@]1(F)O[C@@]([2H])(n2cc(C=O)c(=S)[nH]c2=O)[C@H](O)[C@@H]1O]

### Infectious Diseases

The compounds provided herein can be used to treat viral infectious diseases. Examples of viral infections include but are not limited to, infections caused by RNA viruses (including negative stranded RNA viruses, positive stranded RNA viruses, double stranded RNA viruses and retroviruses) or DNA viruses. All strains, types, and subtypes of RNA viruses and DNA viruses are contemplated herein.

Examples of RNA viruses include, but are not limited to picornaviruses, which include aphthoviruses (for example, foot and mouth disease virus O, A, C, Asia 1, SAT1, SAT2 and SAT3), cardioviruses (for example, encephalomycarditis virus and Theiller's murine encephalomyelitis virus), enteroviruses (for example polioviruses 1, 2 and 3, human enteroviruses A-D, bovine enteroviruses 1 and 2, human coxsackieviruses A1-A22 and A24, human coxsackieviruses B1-B5, human echoviruses 1-7, 9, 11-12, 24, 27, 29-33, human enteroviruses 68-71, porcine enteroviruses 8-10 and simian enteroviruses 1-18), erboviruses (for example, equine rhinitis virus), hepatovirus (for example human hepatitis A virus and simian hepatitis A virus), kobuviruses (for example, bovine kobuvirus and Aichi virus), parechoviruses (for example, human parechovirus 1 and human parechovirus 2), rhinovirus (for example, rhinovirus A, rhinovirus B, rhinovirus C, HRV16, HRV16 (VR-11757), HRV14 (VR-284), or HRV1A (VR-1559), human rhinovirus 1-100 and bovine rhinoviruses 1-3) and teschoviruses (for example, porcine teschovirus).

Additional examples of RNA viruses include caliciviruses, which include noroviruses (for example, Norwalk virus), sapoviruses (for example, Sapporo virus), lagoviruses (for example, rabbit hemorrhagic disease virus and European brown hare syndrome) and vesiviruses (for example vesicular exanthema of swine virus and feline calicivirus). Other RNA viruses include astroviruses, which include mamastorviruses and avastroviruses. Togaviruses are also RNA viruses. Togaviruses include alphaviruses (for example, Chikungunya virus, Sindbis virus, Semliki Forest virus, Western equine encephalitis virus, Eastern Getah virus, Everglades virus, Venezuelan equine encephalitis virus, Ross River virus, Barmah Forest virus and Aura virus) and rubella viruses.

Other examples of RNA viruses are the coronaviruses, which include, human respiratory coronaviruses such as SARS-CoV, SARS-CoV-2, HCoV-229E, HCoV-NL63 and HCoV-OC43. Coronaviruses also include bat SARS-like CoV, Middle East Respiratory Syndrome coronavirus (MERS), turkey coronavirus, chicken coronavirus, feline coronavirus and canine coronavirus. Additional RNA viruses include arteriviruses (for example, equine arterivirus, porcine reproductive and respiratory syndrome virus, lactate dehyrogenase elevating virus of mice and simian hemorraghic fever virus). Other RNA viruses include the rhabdoviruses, which include lyssaviruses (for example, rabies, Lagos bat virus, Mokola virus, Duvenhage virus and European bat lyssavirus), vesiculoviruses (for example, VSV-Indiana, VSV-New Jersey, VSV-Alagoas, Piry virus, Cocal virus, Maraba virus, Isfahan virus and Chandipura virus), and ephemeroviruses (for example, bovine ephemeral fever virus, Adelaide River virus and Berrimah virus). Additional examples of RNA viruses include the filoviruses. These include the Marburg and Ebola viruses (for example, EBOV-Z, EBOV-S, EBOV-IC and EBOV-R).

The paramyxoviruses are also RNA viruses. Examples of these viruses are the rubulaviruses (for example, mumps, parainfluenza virus 5, human parainfluenza virus type 2, Mapuera virus and porcine rubulavirus), avulaviruses (for example, Newcastle disease virus), respoviruses (for example, Sendai virus, human parainfluenza virus type 1 and type 3, bovine parainfluenza virus type 3), henipaviruses (for example, Hendra virus and Nipah virus), morbilloviruses (for example, measles, Cetacean morvilliirus, Canine distemper virus, Peste des-petits-ruminants virus, Phocine distemper virus and Rinderpest virus), pneumoviruses (for example, human respiratory syncytial virus (RSV) A2, B1 and S2, bovine respiratory syncytial virus and pneumonia virus of mice), metapneumoviruses (for example, human metapneumovirus and avian metapneumovirus). Additional paramyxoviruses include Fer-de-Lance virus, Tupaia paramyxovirus, Menangle virus, Tioman virus, Beilong virus, J virus, Mossman virus, Salem virus and Nariva virus.

Additional RNA viruses include the orthomyxoviruses. These viruses include influenza viruses and strains (e.g., influenza A, influenza A strain A/Victoria/3/75, influenza A strain A/Puerto Rico/8/34, influenza A H1N1 (including but not limited to A/WS/33, A/NWS/33 and A/California/04/2009 strains), influenza B, influenza B strain Lee, and influenza C viruses) H2N2, H3N2, H5N1, H7N7, H1N2, H9N2, H7N2, H7N3 and H10N7), as well as avian influenza (for example, strains H5N1, H5N1 Duck/MN/1525/81, H5N2, H7N1, H7N7 and H9N2) thogotoviruses and isaviruses. Orthobunyaviruses (for example, Akabane virus, California encephalitis, Cache Valley virus, Snowshoe hare virus,) nairoviruses (for example, Nairobi sheep virus, Crimean-Congo hemorrhagic fever virus Group and Hughes virus), phleboviruses (for example, Candiru, Punta Toro, Rift Valley Fever, Sandfly Fever, Naples, Toscana, Sicilian and Chagres), and hantaviruses (for example, Hantaan, Dobrava, Seoul, Puumala, Sin Nombre, Bayou, Black Creek Canal, Andes and Thottapalayam) are also RNA viruses. Arenaviruses such as lymphocytic choriomeningitis virus, Lujo virus, Lassa fever virus, Argentine hemorrhagic fever virus, Bolivian hemorrhagic fever virus, Venezuelan hemorrhagic fever virus, SABV and WWAV are also RNA viruses. Borna disease virus is also an RNA virus. Hepatitis D (Delta) virus and hepatitis E are also RNA viruses.

Additional RNA viruses include reoviruses, rotaviruses, birnaviruses, chrysoviruses, cystoviruses, hypoviruses partitiviruses and totoviruses. Orbiviruses such as African horse sickness virus, Blue tongue virus, Changuinola virus, Chenuda virus, Chobar GorgeCorriparta virus, epizootic hemorraghic disease virus, equine encephalosis virus, Eubenangee virus, Ieri virus, Great Island virus, Lebombo virus, Orungo virus, Palyam virus, Peruvian Horse Sickness virus, St. Croix River virus, Umatilla virus, Wad Medani virus, Wallal virus, Warrego virus and Wongorr virus are also RNA viruses. Retroviruses include alpharetroviruses (for example, Rous sarcoma virus and avian leukemia virus), betaretroviruses (for example, mouse mammary tumor virus, Mason-Pfizer monkey virus and Jaagsiekte sheep retrovirus), gammaretroviruses (for example, murine leukemia virus and feline leukemia virus, deltraretroviruses (for example, human T cell leukemia viruses (HTLV-1, HTLV-2), bovine leukemia virus, STLV-1 and STLV-2), epsilonretriviruses (for example, Walleye dermal sarcoma virus and Walleye epidermal hyperplasia virus 1), reticuloendotheliosis virus (for example, chicken syncytial virus, lentiviruses (for example, human immunodeficiency virus (HIV) type 1, human immunodeficiency virus (HIV) type 2, human immunodeficiency virus (HIV) type 3, simian immunodeficiency virus, equine infectious anemia virus, feline immunodeficiency virus, caprine arthritis encephalitis virus and Visna maedi virus) and spumaviruses (for example, human foamy virus and feline syncytia-forming virus).

Examples of DNA viruses include polyomaviruses (for example, simian virus 40, simian agent 12, BK virus, JC virus, Merkel Cell polyoma virus, bovine polyoma virus and lymphotrophic papovavirus), papillomaviruses (for example, human papillomavirus, bovine papillomavirus, adenoviruses (for example, adenoviruses A-F, canine adenovirus type I, canined adeovirus type 2), circoviruses (for example, porcine circovirus and beak and feather disease virus (BFDV)), parvoviruses (for example, canine parvovirus), erythroviruses (for example, adeno-associated virus types 1-8), betaparvoviruses, amdoviruses, densoviruses, iteraviruses, brevidensoviruses, pefudensoviruses, herpes viruses 1,2, 3, 4, 5, 6, 7 and 8 (for example, herpes simplex virus 1, herpes simplex virus 2, varicella-zoster virus, Epstein-Barr virus, cytomegalovirus, Kaposi's sarcoma associated herpes virus, human herpes virus-6 variant A, human herpes virus-6 variant B and cercophithecine herpes virus 1 (B virus)), poxviruses (for example, smallpox (variola), cowpox, monkeypox, vaccinia, Uasin Gishu, camelpox, psuedocowpox, pigeonpox, horsepox, fowlpox, turkeypox and swinepox), and hepadnaviruses (for example, hepatitis B and hepatitis B-like viruses). Chimeric viruses comprising portions of more than one viral genome are also contemplated herein.

In certain embodiments, the disclosure relates to methods of treating or preventing a viral infection comprising administering an effective amount of a compound or pharmaceutical composition disclosed herein to a subject in need thereof. In certain exemplary embodiments, a method of treating or preventing a Zika virus infection is provided, the method comprising administering an effective amount of a compound or pharmaceutical composition disclosed herein to a subject in need thereof.

In certain embodiments, the viral infection is, or is caused by, an alphavirus, flavivirus or coronaviruses orthomyxoviridae or paramyxoviridae, or RSV, influenza, Powassan virus or filoviridae or ebola.

In certain embodiments, the viral infection is, or is caused by, a virus selected from MERS coronavirus, Eastern equine encephalitis virus, Western equine encephalitis virus, Venezuelan equine encephalitis virus, Ross River virus, Barmah Forest virus, Powassan virus, Zika virus, and Chikungunya virus. In certain exemplary embodiments, the viral infection is, or is caused by, a Zika virus.

In certain embodiments, the disclosure relates to methods of treating or preventing a viral infection in the central nervous system (CNS) comprising administering an effective amount of a compound or pharmaceutical composition disclosed herein to a subject in need thereof.

In certain embodiments, the disclosure relates to methods of treating or preventing a viral infection in the lungs comprising administering an effective amount of a compound or pharmaceutical composition disclosed herein to a subject in need thereof.

In certain embodiments, the disclosure relates to methods of treating or preventing a viral infection in the central nervous system (CNS) comprising delivering an effective amount of a compound or pharmaceutical composition disclosed herein to a subject in need thereof.

In certain embodiments, the disclosure relates to methods of treating or preventing a viral infection in the lungs comprising delivering an effective amount of a compound or pharmaceutical composition disclosed herein to a subject in need thereof.

In certain embodiments, the disclosure relates to methods of treating or preventing a viral infection in the central nervous system (CNS) comprising delivering an effective amount of a compound or pharmaceutical composition disclosed herein to the CNS of a subject in need thereof.

In certain embodiments, the disclosure relates to methods of treating or preventing a viral infection in the lungs comprising delivering an effective amount of a compound or pharmaceutical composition disclosed herein to the lungs of a subject in need thereof.

In certain embodiments, the disclosure relates to methods of treating or preventing a coronavirus infection in the CNS comprising administering an effective amount of a compound or pharmaceutical composition disclosed herein to a subject in need thereof.

In certain embodiments, the disclosure relates to methods of treating or preventing a coronavirus infection in the lungs comprising administering an effective amount of a compound or pharmaceutical composition disclosed herein to a subject in need thereof.

In certain embodiments, the disclosure relates to methods of treating or preventing a coronavirus infection in the CNS comprising delivering an effective amount of a compound or pharmaceutical composition disclosed herein to a subject in need thereof.

In certain embodiments, the disclosure relates to methods of treating or preventing a coronavirus infection in the lungs comprising delivering an effective amount of a compound or pharmaceutical composition disclosed herein to a subject in need thereof.

In certain embodiments, the disclosure relates to methods of treating or preventing a coronavirus infection in the CNS comprising delivering an effective amount of a compound or pharmaceutical composition disclosed herein to the CNS of a subject in need thereof.

In certain embodiments, the disclosure relates to methods of treating or preventing a coronavirus infection in the lungs comprising delivering an effective amount of a compound or pharmaceutical composition disclosed herein to the lungs of a subject in need thereof.

In certain embodiments, the disclosure relates to methods of treating or preventing a SARS-CoV-2 infection in the CNS comprising administering an effective amount of a compound or pharmaceutical composition disclosed herein to a subject in need thereof.

In certain embodiments, the disclosure relates to methods of treating or preventing a SARS-CoV-2 infection in the lungs comprising administering an effective amount of a compound or pharmaceutical composition disclosed herein to a subject in need thereof.

In certain embodiments, the disclosure relates to methods of treating or preventing a SARS-CoV-2 infection in the CNS comprising delivering an effective amount of a compound or pharmaceutical composition disclosed herein to a subject in need thereof.

In certain embodiments, the disclosure relates to methods of treating or preventing a SARS-CoV-2 infection in the lungs comprising delivering an effective amount of a compound or pharmaceutical composition disclosed herein to a subject in need thereof.

In certain embodiments, the disclosure relates to methods of treating or preventing a SARS-CoV-2 infection in the CNS comprising delivering an effective amount of a compound or pharmaceutical composition disclosed herein to the CNS of a subject in need thereof.

In certain embodiments, the disclosure relates to methods of treating or preventing a SARS-CoV-2 infection in the lungs comprising delivering an effective amount of a compound or pharmaceutical composition disclosed herein to the lungs of a subject in need thereof.

In certain embodiments, the disclosure relates to methods of treating or preventing a viral infection in the CNS comprising administering an effective amount of EIDD-2749 to a subject in need thereof.

In certain embodiments, the disclosure relates to methods of treating or preventing a viral infection in the lungs comprising administering an effective amount of EIDD-2749 to a subject in need thereof.

In certain embodiments, the disclosure relates to methods of treating or preventing a viral infection in the CNS comprising delivering an effective amount of EIDD-2749 to a subject in need thereof.

In certain embodiments, the disclosure relates to methods of treating or preventing a viral infection in the lungs comprising delivering an effective amount of EIDD-2749 to a subject in need thereof.

In certain embodiments, the disclosure relates to methods of treating or preventing a viral infection in the CNS comprising delivering an effective amount of EIDD-2749 to the CNS of a subject in need thereof.

In certain embodiments, the disclosure relates to methods of treating or preventing a viral infection in the lungs comprising delivering an effective amount of EIDD-2749 to the lungs of a subject in need thereof.

In certain embodiments, the disclosure relates to methods of treating or preventing a coronavirus infection in the CNS comprising administering an effective amount of EIDD-2749 to a subject in need thereof.

In certain embodiments, the disclosure relates to methods of treating or preventing a coronavirus infection in the lungs comprising administering an effective amount of EIDD-2749 to a subject in need thereof.

In certain embodiments, the disclosure relates to methods of treating or preventing a coronavirus infection in the CNS comprising delivering an effective amount of EIDD-2749 to a subject in need thereof.

In certain embodiments, the disclosure relates to methods of treating or preventing a coronavirus infection in the lungs comprising delivering an effective amount of EIDD-2749 to a subject in need thereof.

In certain embodiments, the disclosure relates to methods of treating or preventing a coronavirus infection in the CNS comprising delivering an effective amount of EIDD-2749 to the CNS of a subject in need thereof.

In certain embodiments, the disclosure relates to methods of treating or preventing a coronavirus infection in the lungs comprising delivering an effective amount of EIDD-2749 to the lungs of a subject in need thereof.

In certain embodiments, the disclosure relates to methods of treating or preventing a SARS-CoV-2 infection in the CNS comprising administering an effective amount of EIDD-2749 to a subject in need thereof.

In certain embodiments, the disclosure relates to methods of treating or preventing a SARS-CoV-2 infection in the lungs comprising administering an effective amount of EIDD-2749 to a subject in need thereof.

In certain embodiments, the disclosure relates to methods of treating or preventing a SARS-CoV-2 infection in the CNS comprising delivering an effective amount of EIDD-2749 to a subject in need thereof.

In certain embodiments, the disclosure relates to methods of treating or preventing a SARS-CoV-2 infection in the lungs comprising delivering an effective amount of EIDD-2749 to a subject in need thereof.

In certain embodiments, the disclosure relates to methods of treating or preventing a SARS-CoV-2 infection in the CNS comprising delivering an effective amount of EIDD-2749 to the CNS of a subject in need thereof.

In certain embodiments, the disclosure relates to methods of treating or preventing a SARS-CoV-2 infection in the lungs comprising delivering an effective amount of EIDD-2749 to the lungs of a subject in need thereof.

In certain embodiments, the compound is administered by inhalation through the lungs.

In some embodiments, the subject is at risk of, exhibiting symptoms of, or diagnosed with influenza A virus including subtype H1N1, H3N2, H7N9, or H5N1, influenza B virus, influenza C virus, rotavirus A, rotavirus B, rotavirus C, rotavirus D, rotavirus E, human coronavirus, SARS coronavirus, MERS coronavirus, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, norovirus, Rubella virus, lymphocytic choriomeningitis virus (LCMV), Dengue virus, Zika virus, chikungunya, Eastern equine encephalitis virus (EEEV), Western equine encephalitis virus (WEEV), Venezuelan equine encephalitis virus (VEEV), Ross River virus, Barmah Forest virus, yellow fever virus, measles virus, mumps virus, respiratory syncytial virus, rinderpest virus, California encephalitis virus, hantavirus, rabies virus, ebola virus, marburg virus, herpes simplex virus-1 (HSV-1), herpes simplex virus-2 (HSV-2), varicella zoster virus (VZV), Epstein-Barr virus (EBV), cytomegalovirus (CMV), herpes lymphotropic virus, roseolovirus, or Kaposi's sarcoma-associated herpesvirus, hepatitis A, hepatitis B, hepatitis C, hepatitis D, hepatitis E or human immunodeficiency virus (HIV), The Human T-lymphotropic virus Type I (HTLV-1), Friend spleen focus-forming virus (SFFV) or Xenotropic MuLV-Related Virus (XMRV). In some embodiments, the subject is at risk of, exhibiting symptoms of, or diagnosed with a Zika virus infection.

In certain embodiments, the subject is diagnosed with influenza A virus including subtypes H1N1, H3N2, H7N9, H5N1 (low path), and H5N1 (high path) influenza B virus, influenza C virus, rotavirus A, rotavirus B, rotavirus C, rotavirus D, rotavirus E, SARS coronavirus, MERS-CoV, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, norovirus, Rubella virus, lymphocytic choriomeningitis virus (LCMV), yellow fever virus, measles virus, mumps virus, respiratory syncytial virus, parainfluenza viruses 1 and 3, rinderpest virus, chikungunya, eastern equine encephalitis virus (EEEV), Venezuelan equine encephalitis virus (VEEV), western equine encephalitis virus (WEEV), California encephalitis virus, Japanese encephalitis virus, Rift Valley fever virus (RVFV), hantavirus, Dengue virus serotypes 1, 2, 3 and 4, Zika virus, West Nile virus, Tacaribe virus, Junin, rabies virus, ebola virus, marburg virus, adenovirus, herpes simplex virus-1 (HSV-1), herpes simplex virus-2 (HSV-2), varicella zoster virus (VZV), Epstein-Barr virus (EBV), cytomegalovirus (CMV), herpes lymphotropic virus, roseolovirus, or Kaposi's sarcoma-associated herpesvirus, hepatitis A, hepatitis B, hepatitis C, hepatitis D, hepatitis E or human immunodeficiency virus (HIV). In certain embodiments, the subject is diagnosed with a Zika virus infection.

In certain embodiments, the subject is diagnosed with gastroenteritis, acute respiratory disease, severe acute respiratory syndrome, post-viral fatigue syndrome, viral hemorrhagic fevers, acquired immunodeficiency syndrome or hepatitis.

In some embodiments, the disclosure relates to treating or preventing an infection by viruses, bacteria, fungi, protozoa, and parasites. In some embodiments, the disclosure relates to methods of treating a viral infection comprising administering a compound herein to a subject that is diagnosed with, suspected of, or exhibiting symptoms of a viral infection.

Viruses are infectious agents that can typically replicate inside the living cells of organisms. Virus particles (virions) usually consist of nucleic acids, a protein coat, and in some cases an envelope of lipids that surrounds the protein coat. The shapes of viruses range from simple helical and icosahedral forms to more complex structures. Virally coded protein subunits will self-assemble to form a capsid, generally requiring the presence of the virus genome. Complex viruses can code for proteins that assist in the construction of their capsid. Proteins associated with nucleic acid are known as nucleoproteins, and the association of viral capsid proteins with viral nucleic acid is called a nucleocapsid.

Viruses are transmitted by a variety of methods including direct or bodily fluid contact, e.g., blood, tears, semen, preseminal fluid, saliva, milk, vaginal secretions, lesions; droplet contact, fecal-oral contact, or as a result of an animal bite or birth. A virus has either DNA or RNA genes and is called a DNA virus or a RNA virus respectively. A viral genome is either single-stranded or double-stranded. Some viruses contain a genome that is partially double-stranded and partially single-stranded. For viruses with RNA or single-stranded DNA, the strands are said to be either positive-sense (called the plus-strand) or negative-sense (called the minus-strand), depending on whether it is complementary to the viral messenger RNA (mRNA). Positive-sense viral RNA is identical to viral mRNA and thus can be immediately translated by the host cell. Negative-sense viral RNA is complementary to mRNA and thus must be converted to positive-sense RNA by an RNA polymerase before translation. DNA nomenclature is similar to RNA nomenclature, in that the coding strand for the viral mRNA is complementary to it (negative), and the non-coding strand is a copy of it (positive).

Antigenic shift, or reassortment, can result in novel strains. Viruses undergo genetic change by several mechanisms. These include a process called genetic drift where individual bases in the DNA or RNA mutate to other bases. Antigenic shift occurs when there is a major change in the genome of the virus. This can be a result of recombination or reassortment. RNA viruses often exist as quasispecies or swarms of viruses of the same species but with slightly different genome nucleoside sequences.

The genetic material within viruses, and the method by which the material is replicated, vary between different types of viruses. The genome replication of most DNA viruses takes place in the nucleus of the cell. If the cell has the appropriate receptor on its surface, these viruses enter the cell by fusion with the cell membrane or by endocytosis.

Most DNA viruses are entirely dependent on the host DNA and RNA synthesizing machinery, and RNA processing machinery. Replication usually takes place in the cytoplasm. RNA viruses typically use their own RNA replicase enzymes to create copies of their genomes.

The Baltimore classification of viruses is based on the mechanism of mRNA production. Viruses must generate mRNAs from their genomes to produce proteins and replicate themselves, but different mechanisms are used to achieve this. Viral genomes may be single-stranded (ss) or double-stranded (ds), RNA or DNA, and may or may not use reverse transcriptase (RT). Additionally, ssRNA viruses may be either sense (plus) or antisense (minus). This classification places viruses into seven groups: I, dsDNA viruses (e.g. adenoviruses, herpesviruses, poxviruses); II, ssDNA viruses (plus)sense DNA (e.g. parvoviruses); III, dsRNA viruses (e.g. reoviruses); IV, (plus)ssRNA viruses (plus)sense RNA (e.g. picornaviruses, togaviruses); V, (minus)ssRNA viruses (minus)sense RNA (e.g. orthomyxoviruses, Rhabdoviruses); VI, ssRNA-RT viruses (plus)sense RNA with DNA intermediate in life-cycle (e.g. retroviruses); and VII, dsDNA-RT viruses (e.g. hepadnaviruses).

Human immunodeficiency virus (HIV) is a lentivirus (a member of the retrovirus family) that causes acquired immunodeficiency syndrome (AIDS). Lentiviruses are transmitted as single-stranded, positive-sense, enveloped RNA viruses. Upon entry of the target cell, the viral RNA genome is converted to double-stranded DNA by a virally encoded reverse transcriptase. This viral DNA is then integrated into the cellular DNA by a virally encoded integrase, along with host cellular co-factors. There are two species of HIV. HIV-1 is sometimes termed LAV or HTLV-III.

HIV infects primarily vital cells in the human immune system such as helper T cells (CD4+ T cells), macrophages, and dendritic cells. HIV infection leads to low levels of CD4+ T cells. When CD4+ T cell numbers decline below a critical level, cell-mediated immunity is lost, and the body becomes progressively more susceptible to other viral or bacterial infections. Subjects with HIV typically develop malignancies associated with the progressive failure of the immune system.

The viral envelope is composed of two layers of phospholipids taken from the membrane of a human cell when a newly formed virus particle buds from the cell. Embedded in the viral envelope are proteins from the host cell and a HIV protein known as Env. Env contains glycoproteinsgp120, and gp41. The RNA genome consists of at structural landmarks (LTR, TAR, RRE, PE, SLIP, CRS, and INS) and nine genes (gag, pol, and env, tat, rev, nef, vif, vpr, vpu, and sometimes a tenth tev, which is a fusion of tat env and rev) encoding 19 proteins. Three of these genes, gag, pol, and env, contain information needed to make the structural proteins for new virus particles. HIV-1 diagnosis is typically done with antibodies in an ELISA, Western blot, or immunoaffinity assays or by nucleic acid testing (e.g., viral RNA or DNA amplification).

HIV is typically treated with a combination of antiviral agent, e.g., two nucleoside-analogue reverse transcription inhibitors and one non-nucleoside-analogue reverse transcription inhibitor or protease inhibitor. The three-drug combination is commonly known as a triple cocktail. In certain embodiments, the disclosure relates to treating a subject diagnosed with HIV by administering a pharmaceutical composition disclosed herein in combination with two nucleoside-analogue reverse transcription inhibitors and one non-nucleoside-analogue reverse transcription inhibitor or protease inhibitor.

In certain embodiments, the disclosure relates to treating a subject by administering a compound disclosed herein, emtricitabine, tenofovir, and efavirenz. In certain embodiments, the disclosure relates to treating a subject by administering a compound disclosed herein, emtricitabine, tenofovir and raltegravir. In certain embodiments, the disclosure relates to treating a subject by administering a compound disclosed herein, emtricitabine, tenofovir, ritonavir and darunavir. In certain embodiments, the disclosure relates to treating a subject by administering a compound disclosed herein, emtricitabine, tenofovir, ritonavir and atazanavir.

Banana lectin (BanLec or BanLec-1) is one of the predominant proteins in the pulp of ripe bananasand has binding specificity for mannose and mannose-containing oligosaccharides. BanLec binds to the HIV-1 envelope protein gp120. In certain embodiments, the disclosure relates to treating viral infections, such as HIV, by administering a compound disclosed herein in combination with a banana lectin.

Therapeutic agents in some cases may suppress the virus for a long period of time. Typical medications are a combination of interferon alpha and ribavirin. Subjects may receive injections of pegylated interferon alpha. Genotypes 1 and 4 are less responsive to interferon-based treatment than are the other genotypes (2, 3, 5 and 6). In certain embodiments, the disclosure relates to treating a subject with HCV by administering a compound disclosed herein to a subject exhibiting symptoms or diagnosed with HCV. In certain embodiments, the compound is administered in combination with interferon alpha and another antiviral agent such as ribavirin, and/or a protease inhibitor such as telaprevir or boceprevir. In certain embodiments, the subject is diagnosed with genotype 2, 3, 5, or 6. In other embodiments, the subject is diagnosed with genotype 1 or 4.

In certain embodiments, the subject is diagnosed to have a virus by nucleic acid detection or viral antigen detection. Cytomegalovirus (CMV) belongs to the Betaherpesvirinae subfamily of Herpesviridae. In humans it is commonly known as HCMV or Human Herpesvirus 5 (HHV-5). Herpesviruses typically share a characteristic ability to remain latent within the body over long periods. HCMV infection may be life threatening for patients who are immunocompromised. In certain embodiments, the disclosure relates to methods of treating a subject diagnosed with cytomegalovirus or preventing a cytomegalovirus infection by administration of a compound disclosed herein. In certain embodiments, the subject is immunocompromised. In typical embodiments, the subject is an organ transplant recipient, undergoing hemodialysis, diagnosed with cancer, receiving an immunosuppressive drug, and/or diagnosed with an HIV-infection. In certain embodiments, the subject may be diagnosed with cytomegalovirus hepatitis, the cause of fulminant liver failure, cytomegalovirus retinitis (inflammation of the retina, may be detected by ophthalmoscopy), cytomegalovirus colitis (inflammation of the large bowel), cytomegalovirus pneumonitis, cytomegalovirus esophagitis, cytomegalovirus mononucleosis, polyradiculopathy, transverse myelitis, and subacute encephalitis. In certain embodiments, a compound disclosed herein is administered in combination with an antiviral agent such as valganciclovir or ganciclovir. In certain embodiments, the subject undergoes regular serological monitoring.

HCMV infections of a pregnant subject may lead to congenital abnormalities. Congenital HCMV infection occurs when the mother suffers a primary infection (or reactivation) during pregnancy. In certain embodiments, the disclosure relates to methods of treating a pregnant subject diagnosed with cytomegalovirus or preventing a cytomegalovirus infection in a subject at risk for, attempting to become, or currently pregnant by administering compound disclosed herein.

Subjects who have been infected with CMV typically develop antibodies to the virus. A number of laboratory tests that detect these antibodies to CMV have been developed. The virus may be cultured from specimens obtained from urine, throat swabs, bronchial lavages and tissue samples to detect active infection. One may monitor the viral load of CMV-infected subjects using PCR. CMV pp65 antigenemia test is an immunoaffinity based assay for identifying the pp65 protein of cytomegalovirus in peripheral blood leukocytes. CMV should be suspected if a patient has symptoms of infectious mononucleosis but has negative test results for mononucleosis and Epstein-Barr virus, or if they show signs of hepatitis, but have negative test results for hepatitis A, B, and C. A virus culture can be performed at any time the subject is symptomatic. Laboratory testing for antibody to CMV can be performed to determine if a subject has already had a CMV infection.

The enzyme-linked immunosorbent assay (or ELISA) is the most commonly available serologic test for measuring antibody to CMV. The result can be used to determine if acute infection, prior infection, or passively acquired maternal antibody in an infant is present. Other tests include various fluorescence assays, indirect hemagglutination, (PCR), and latex agglutination. An ELISA technique for CMV-specific IgM is available.

Hepatitis B virus is a hepadnavirus. The virus particle, (virion) consists of an outer lipid envelope and an icosahedral nucleocapsid core composed of protein. The genome of HBV is made of circular DNA, but the DNA is not fully double-stranded. One end of the strand is linked to the viral DNA polymerase. The virus replicates through an RNA intermediate form by reverse transcription. Replication typically takes place in the liver where it causes inflammation (hepatitis). The virus spreads to the blood where virus-specific proteins and their corresponding antibodies are found in infected people. Blood tests for these proteins and antibodies are used to diagnose the infection.

Hepatitis B virus gains entry into the cell by endocytosis. Because the virus multiplies via RNA made by a host enzyme, the viral genomic DNA has to be transferred to the cell nucleus by host chaperones. The partially double stranded viral DNA is then made fully double stranded and transformed into covalently closed circular DNA (cccDNA) that serves as a template for transcription of viral mRNAs. The virus is divided into four major serotypes (adr, adw, ayr, ayw) based on antigenic epitopes presented on its envelope proteins, and into eight genotypes (A-H) according to overall nucleotide sequence variation of the genome.

The hepatitis B surface antigen (HBsAg) is typically used to screen for the presence of this infection. It is the first detectable viral antigen to appear during infection. However, early in an infection, this antigen may not be present and it may be undetectable later in the infection if it is being cleared by the host. The infectious virion contains an inner “core particle” enclosing viral genome. The icosahedral core particle is made of core protein, alternatively known as hepatitis B core antigen, or HBcAg. IgM antibodies to the hepatitis B core antigen (anti-HBc IgM) may be used as a serological marker. Hepatitis B e antigen (HBeAg) may appear. The presence of HBeAg in the serum of the host is associated with high rates of viral replication. Certain variants of the hepatitis B virus do not produce the ‘e’ antigen, If the host is able to clear the infection, typically the HBsAg will become undetectable and will be followed by IgG antibodies to the hepatitis B surface antigen and core antigen, (anti-HBs and anti HBc IgG). The time between the removal of the HBsAg and the appearance of anti-HBs is called the window period. A person negative for HBsAg but positive for anti-HBs has either cleared an infection or has been vaccinated previously. Individuals who remain HBsAg positive for at least six months are considered to be hepatitis B carriers. Carriers of the virus may have chronic hepatitis B, which would be reflected by elevated serum alanine aminotransferase levels and inflammation of the liver that may be identified by biopsy. Nucleic acid (PCR) tests have been developed to detect and measure the amount of HBV DNA in clinical specimens.

Acute infection with hepatitis B virus is associated with acute viral hepatitis. Acute viral hepatitis typically begins with symptoms of general ill health, loss of appetite, nausea, vomiting, body aches, mild fever, dark urine, and then progresses to development of jaundice. Chronic infection with hepatitis B virus may be either asymptomatic or may be associated with a chronic inflammation of the liver (chronic hepatitis), possibly leading to cirrhosis. Having chronic hepatitis B infection increases the incidence of hepatocellular carcinoma (liver cancer).

During HBV infection, the host immune response causes both hepatocellular damage and viral clearance. The adaptive immune response, particularly virus-specific cytotoxic T lymphocytes (CTLs), contributes to most of the liver injury associated with HBV infection. By killing infected cells and by producing antiviral cytokines capable of purging HBV from viable hepatocytes, CTLs eliminate the virus. Although liver damage is initiated and mediated by the CTLs, antigen-nonspecific inflammatory cells can worsen CTL-induced immunopathology, and platelets activated at the site of infection may facilitate the accumulation of CTLs in the liver.

Therapeutic agents can stop the virus from replicating, thus minimizing liver damage. In certain embodiments, the disclosure relates to methods of treating a subject diagnosed with HBV by administering a compound disclosed herein. In certain embodiments, the subject is immunocompromised. In certain embodiments, the compound is administered in combination with another antiviral agent such as lamivudine, adefovir, tenofovir, telbivudine, and entecavir, and/or immune system modulators interferon alpha-2a and pegylated interferon alpha-2a (Pegasys). In certain embodiments, the disclosure relates to preventing an HBV infection in an immunocompromised subject at risk of infection by administering a pharmaceutical composition disclosed herein and optionally one or more antiviral agents. In certain embodiments, the subject is at risk of an infection because the sexual partner of the subject is diagnosed with HBV.

In certain embodiments, pharmaceutical compositions disclosed herein are administered in combination with a second antiviral agent, such as ABT-450, ABT-267, ABT-333, ABT-493, ABT-530, abacavir, acyclovir, acyclovir, adefovir, amantadine, amprenavir, ampligen, arbidol, atazanavir, atripla, boceprevir, cidofovir, combivir, daclatasvir, darunavir, dasabuvir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, tipranavir, trifluridine, trizivir, tromantadine, truvada, valaciclovir, valganciclovir, vicriviroc, vidarabine, viramidine zalcitabine, zanamivir, or zidovudine and combinations thereof.

In certain embodiments, pharmaceutical compositions disclosed herein can be coformulated and administered in combination with a second antiviral agent selected from:

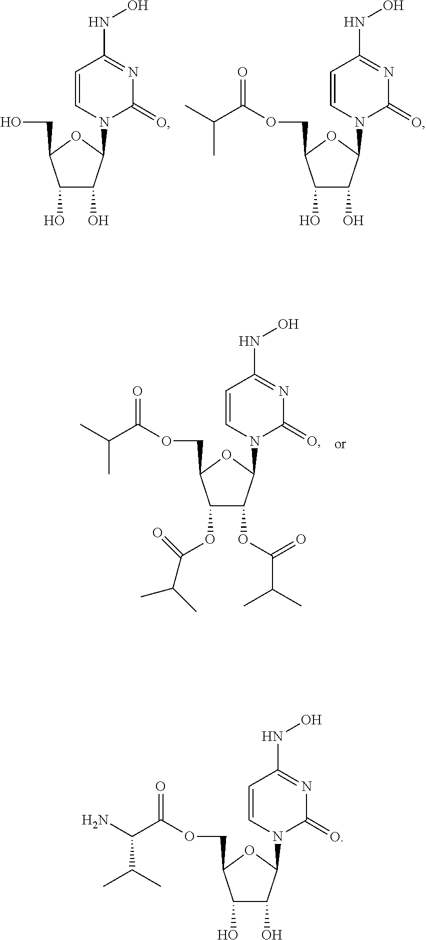

In certain embodiments,

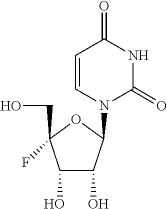 [SMILES: O=c1ccn([C@@H]2O[C@](F)(CO)[C@@H](O)[C@H]2O)c(=O)[nH]1]

can be coformulated and administered in combination with a second antiviral agent selected from:

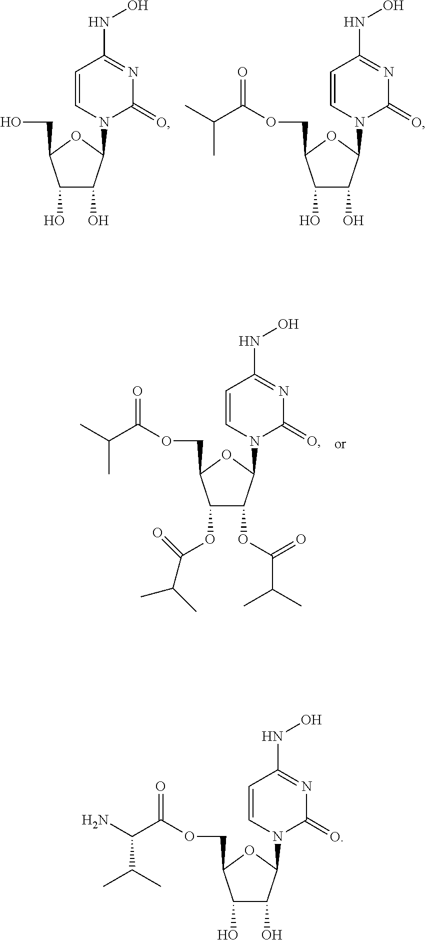

In certain embodiments,

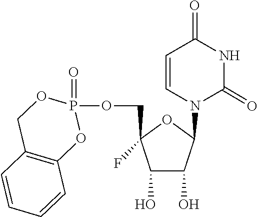 [SMILES: O=c1ccn([C@@H]2O[C@](F)(COP3(=O)OCc4ccccc4O3)[C@@H](O)[C@H]2O)c(=O)[nH]1]

can be coformulated and administered in combination with a second antiviral agent selected from:

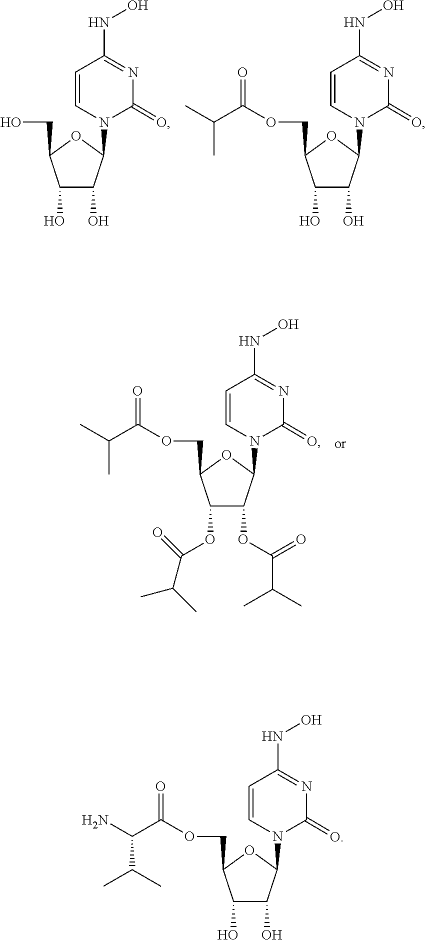

In certain embodiments, pharmaceutical compositions disclosed herein can be coformulated and administered in combination with a second antiviral agent selected from WO 2016/106050 or WO 2017/156380.

In certain embodiments,

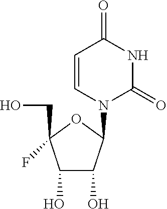 [SMILES: O=c1ccn([C@@H]2O[C@](F)(CO)[C@@H](O)[C@H]2O)c(=O)[nH]1]

can be coformulated and administered in combination with a second antiviral agent selected from WO 2016/106050 or WO 2017/156380.

In certain embodiments,

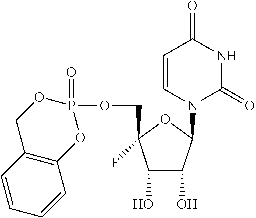 [SMILES: O=c1ccn([C@@H]2O[C@](F)(COP3(=O)OCc4ccccc4O3)[C@@H](O)[C@H]2O)c(=O)[nH]1]

can be coformulated and administered in combination with a second antiviral agent selected from WO 2016/106050 or WO 2017/156380.

In exemplified embodiments,

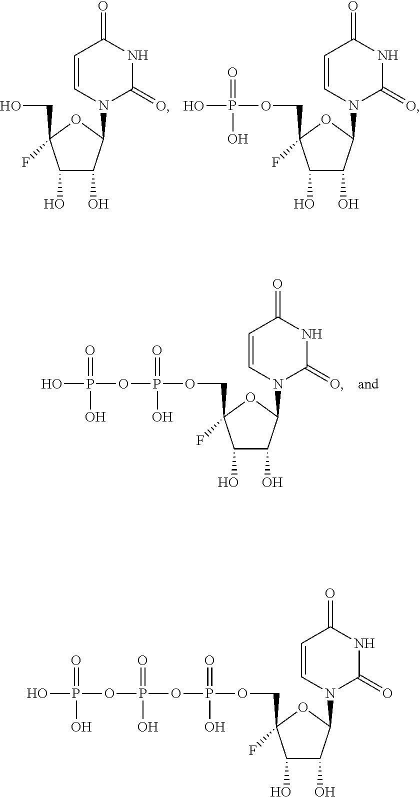

can be combined with

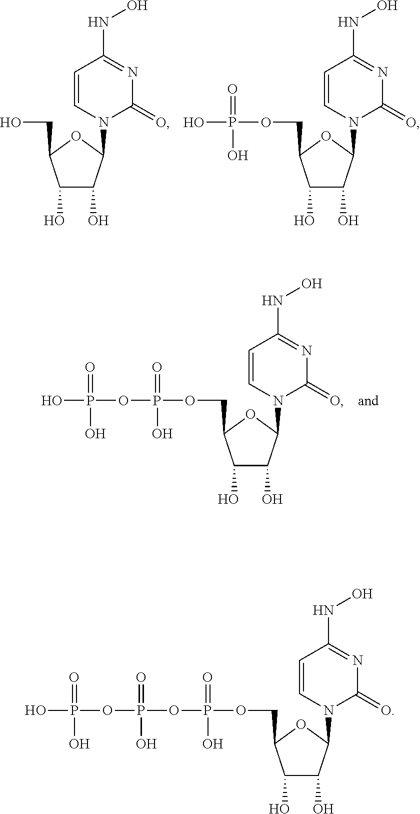

In exemplified embodiments,

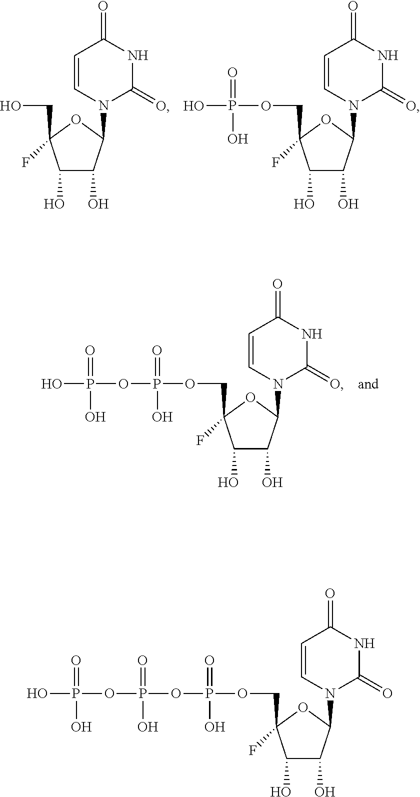

can be combined with

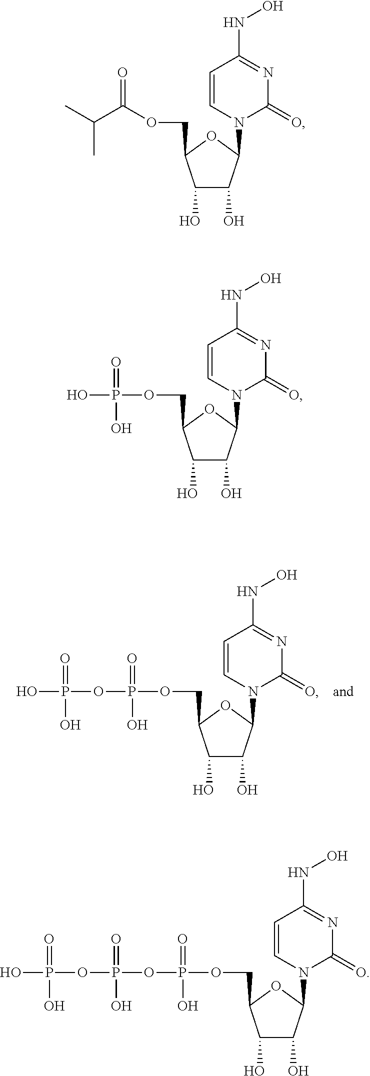 [SMILES: CC(C)C(=O)OC[C@H]1O[C@@H](n2ccc(NO)nc2=O)[C@H](O)[C@@H]1O ; O=c1nc(NO)ccn1[C@@H]1O[C@H](COP(=O)(O)O)[C@@H](O)[C@H]1O ; O=c1nc(NO)ccn1[C@@H]1O[C@H](COP(=O)(O)OP(=O)(O)O)[C@@H](O)[C@H]1O ; O=c1nc(NO)ccn1[C@@H]1O[C@H](COP(=O)(O)OP(=O)(O)OP(=O)(O)O)[C@@H](O)[C@H]1O]

In exemplified embodiments,

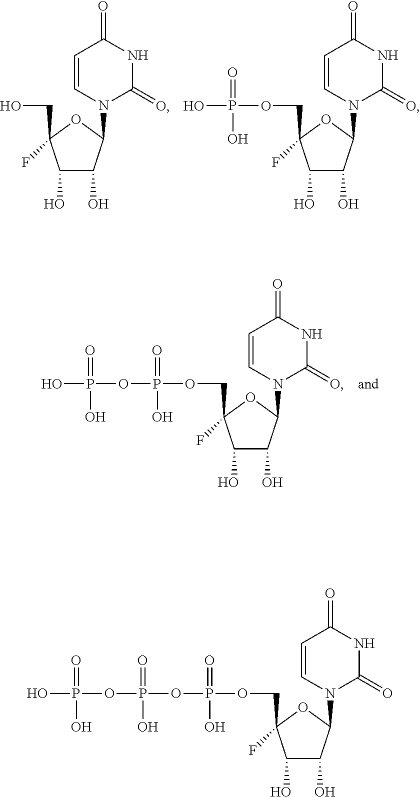

can be combined with

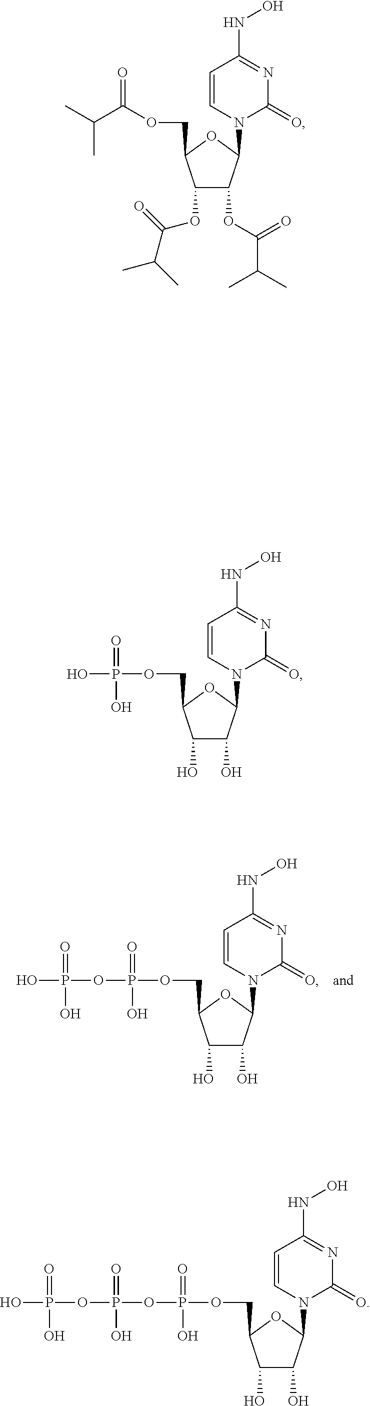 [SMILES: CC(C)C(=O)OC[C@H]1O[C@@H](n2ccc(NO)nc2=O)[C@H](OC(=O)C(C)C)[C@@H]1OC(=O)C(C)C ; O=c1nc(NO)ccn1[C@@H]1O[C@H](COP(=O)(O)O)[C@@H](O)[C@H]1O ; O=c1nc(NO)ccn1[C@@H]1O[C@H](COP(=O)(O)OP(=O)(O)O)[C@@H](O)[C@H]1O ; O=c1nc(NO)ccn1[C@@H]1O[C@H](COP(=O)(O)OP(=O)(O)OP(=O)(O)O)[C@@H](O)[C@H]1O]

In exemplified embodiments,

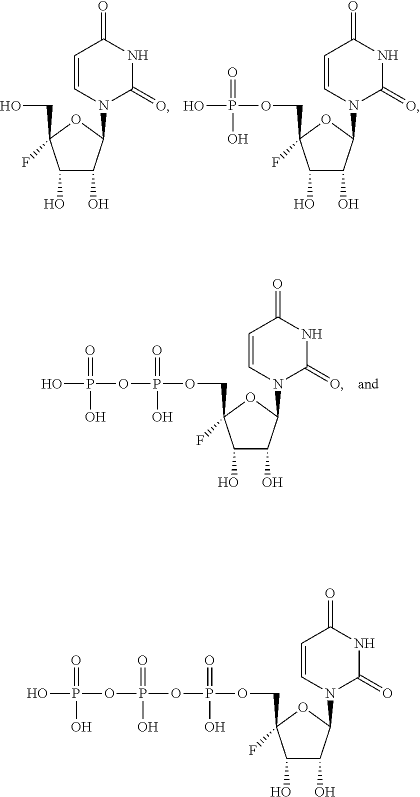

can be combined with

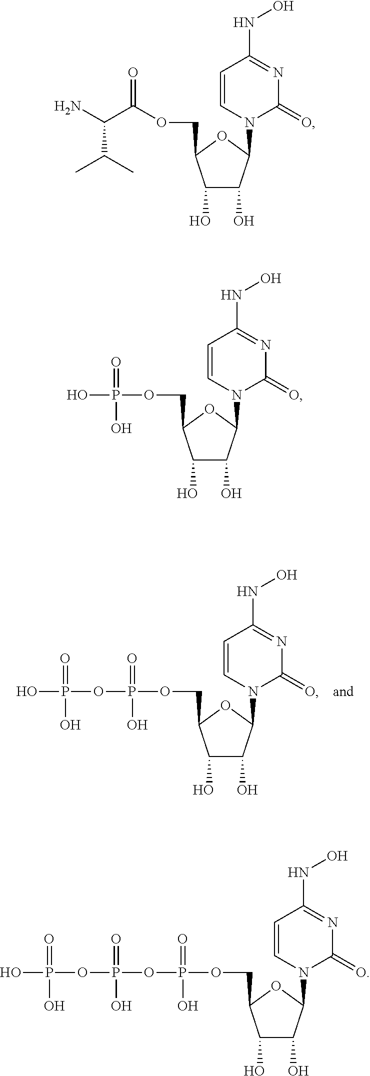 [SMILES: CC(C)[C@H](N)C(=O)OC[C@H]1O[C@@H](n2ccc(NO)nc2=O)[C@H](O)[C@@H]1O ; O=c1nc(NO)ccn1[C@@H]1O[C@H](COP(=O)(O)O)[C@@H](O)[C@H]1O ; O=c1nc(NO)ccn1[C@@H]1O[C@H](COP(=O)(O)OP(=O)(O)O)[C@@H](O)[C@H]1O ; O=c1nc(NO)ccn1[C@@H]1O[C@H](COP(=O)(O)OP(=O)(O)OP(=O)(O)O)[C@@H](O)[C@H]1O]

In exemplified embodiments,

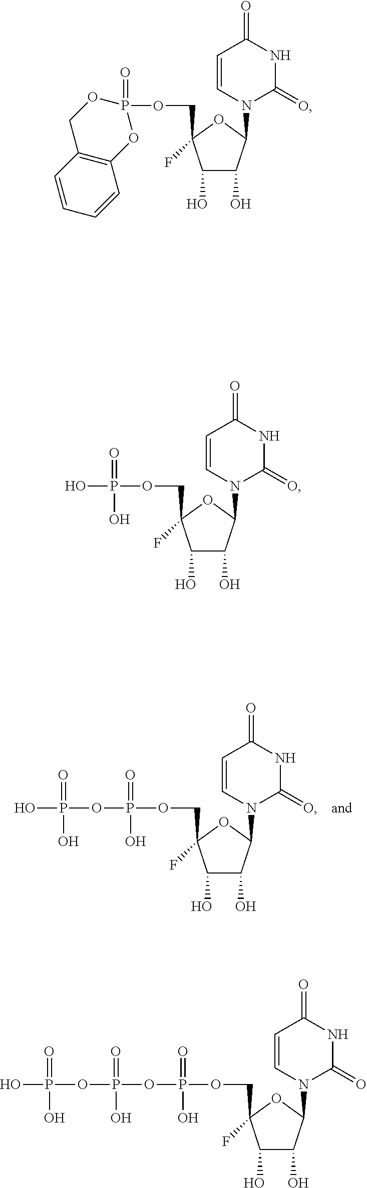 [SMILES: O=c1ccn([C@@H]2O[C@](F)(COP3(=O)OCc4ccccc4O3)[C@@H](O)[C@H]2O)c(=O)[nH]1 ; O=c1ccn([C@@H]2O[C@](F)(COP(=O)(O)O)[C@@H](O)[C@H]2O)c(=O)[nH]1 ; O=c1ccn([C@@H]2O[C@](F)(COP(=O)(O)OP(=O)(O)O)[C@@H](O)[C@H]2O)c(=O)[nH]1 ; O=c1ccn([C@@H]2O[C@](F)(COP(=O)(O)OP(=O)(O)OP(=O)(O)O)[C@@H](O)[C@H]2O)c(=O)[nH]1]

can be combined with

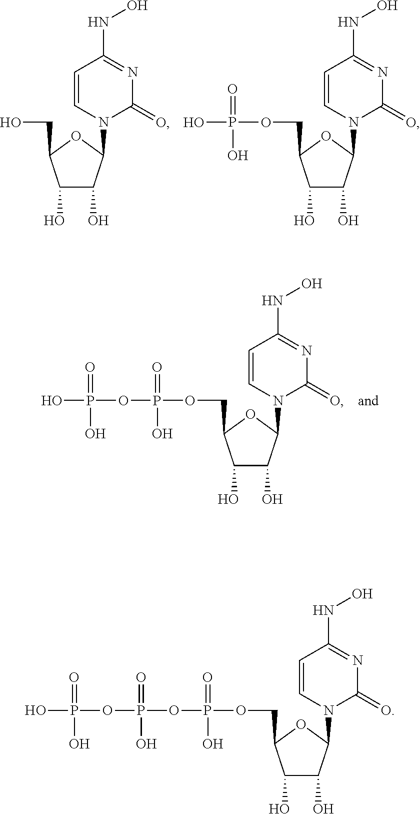

In exemplified embodiments,

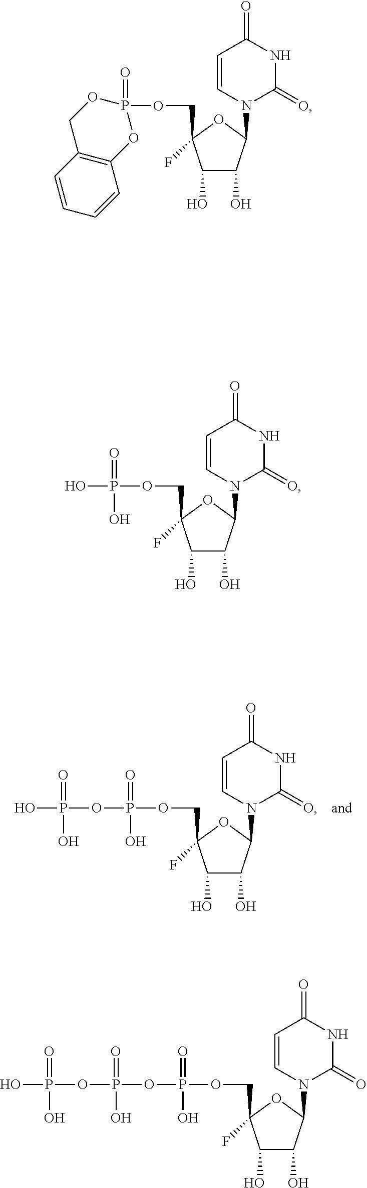 [SMILES: O=c1ccn([C@@H]2O[C@](F)(COP3(=O)OCc4ccccc4O3)[C@@H](O)[C@H]2O)c(=O)[nH]1 ; O=c1ccn([C@@H]2O[C@](F)(COP(=O)(O)O)[C@@H](O)[C@H]2O)c(=O)[nH]1 ; O=c1ccn([C@@H]2O[C@](F)(COP(=O)(O)OP(=O)(O)O)[C@@H](O)[C@H]2O)c(=O)[nH]1 ; O=c1ccn([C@@H]2O[C@](F)(COP(=O)(O)OP(=O)(O)OP(=O)(O)O)[C@@H](O)[C@H]2O)c(=O)[nH]1]

can be combined with

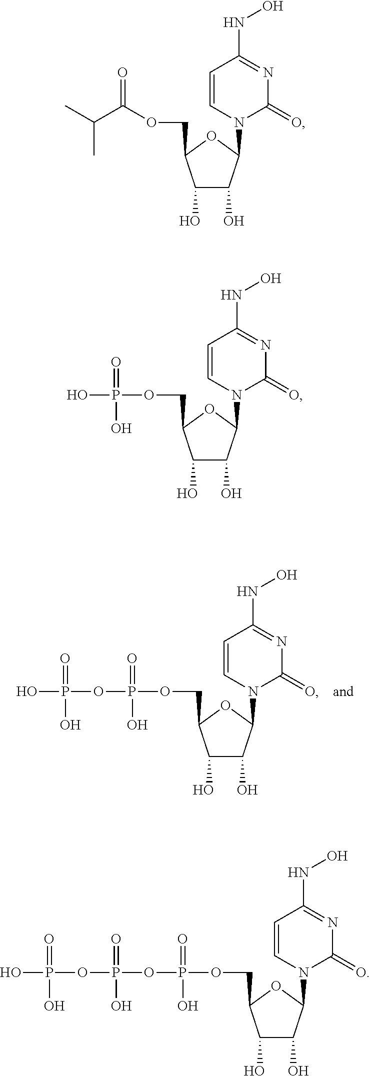 [SMILES: CC(C)C(=O)OC[C@H]1O[C@@H](n2ccc(NO)nc2=O)[C@H](O)[C@@H]1O ; O=c1nc(NO)ccn1[C@@H]1O[C@H](COP(=O)(O)O)[C@@H](O)[C@H]1O ; O=c1nc(NO)ccn1[C@@H]1O[C@H](COP(=O)(O)OP(=O)(O)O)[C@@H](O)[C@H]1O ; O=c1nc(NO)ccn1[C@@H]1O[C@H](COP(=O)(O)OP(=O)(O)OP(=O)(O)O)[C@@H](O)[C@H]1O]

In exemplified embodiments,

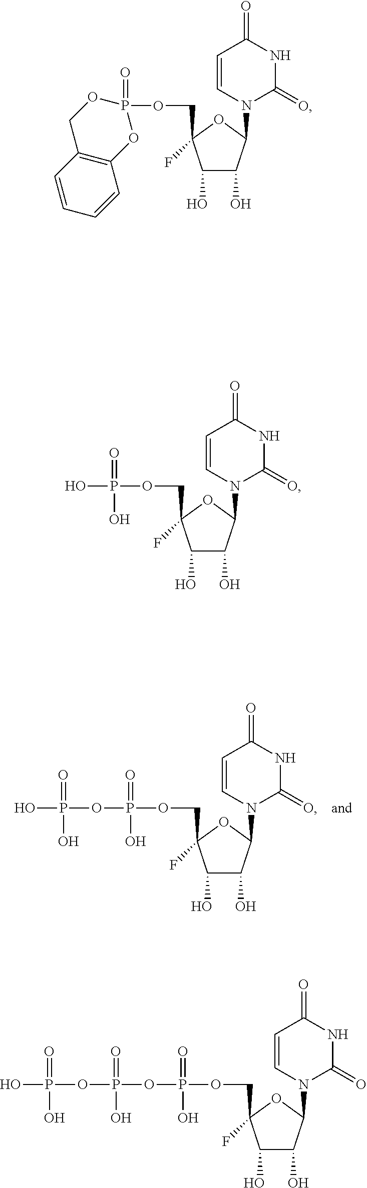 [SMILES: O=c1ccn([C@@H]2O[C@](F)(COP3(=O)OCc4ccccc4O3)[C@@H](O)[C@H]2O)c(=O)[nH]1 ; O=c1ccn([C@@H]2O[C@](F)(COP(=O)(O)O)[C@@H](O)[C@H]2O)c(=O)[nH]1 ; O=c1ccn([C@@H]2O[C@](F)(COP(=O)(O)OP(=O)(O)O)[C@@H](O)[C@H]2O)c(=O)[nH]1 ; O=c1ccn([C@@H]2O[C@](F)(COP(=O)(O)OP(=O)(O)OP(=O)(O)O)[C@@H](O)[C@H]2O)c(=O)[nH]1]

can be combined with

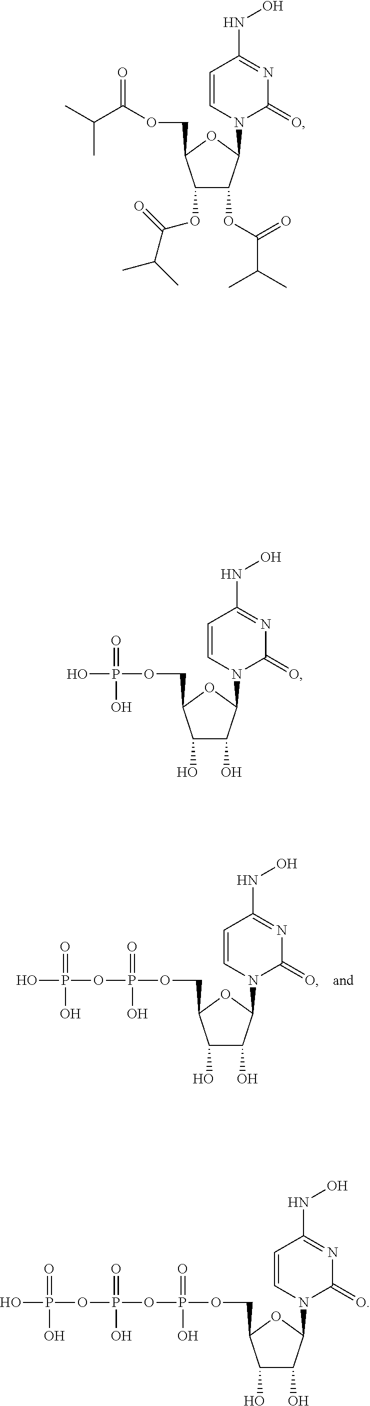 [SMILES: CC(C)C(=O)OC[C@H]1O[C@@H](n2ccc(NO)nc2=O)[C@H](OC(=O)C(C)C)[C@@H]1OC(=O)C(C)C ; O=c1nc(NO)ccn1[C@@H]1O[C@H](COP(=O)(O)O)[C@@H](O)[C@H]1O ; O=c1nc(NO)ccn1[C@@H]1O[C@H](COP(=O)(O)OP(=O)(O)O)[C@@H](O)[C@H]1O ; O=c1nc(NO)ccn1[C@@H]1O[C@H](COP(=O)(O)OP(=O)(O)OP(=O)(O)O)[C@@H](O)[C@H]1O]

In exemplified embodiments,

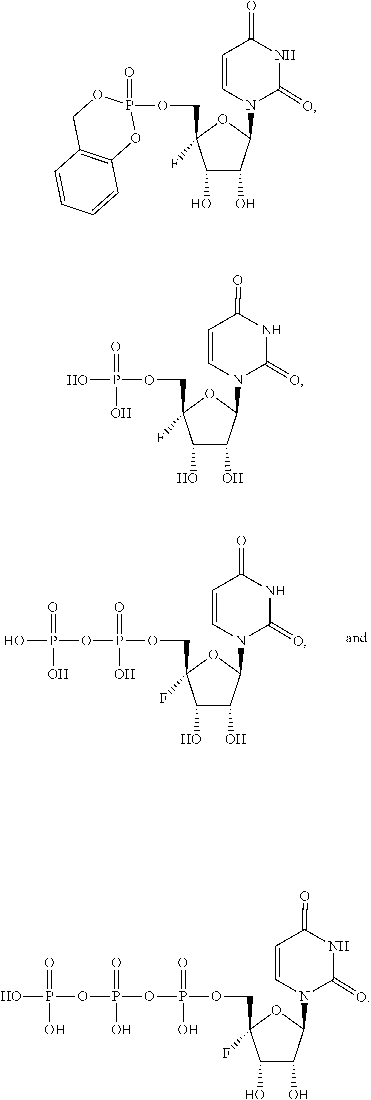 [SMILES: O=c1ccn([C@@H]2O[C@](F)(COP3(=O)OCc4ccccc4O3)[C@@H](O)[C@H]2O)c(=O)[nH]1 ; O=c1ccn([C@@H]2O[C@](F)(COP(=O)(O)O)[C@@H](O)[C@H]2O)c(=O)[nH]1 ; O=c1ccn([C@@H]2O[C@](F)(COP(=O)(O)OP(=O)(O)O)[C@@H](O)[C@H]2O)c(=O)[nH]1 ; O=c1ccn([C@@H]2O[C@](F)(COP(=O)(O)OP(=O)(O)OP(=O)(O)O)[C@@H](O)[C@H]2O)c(=O)[nH]1]

can be combined with

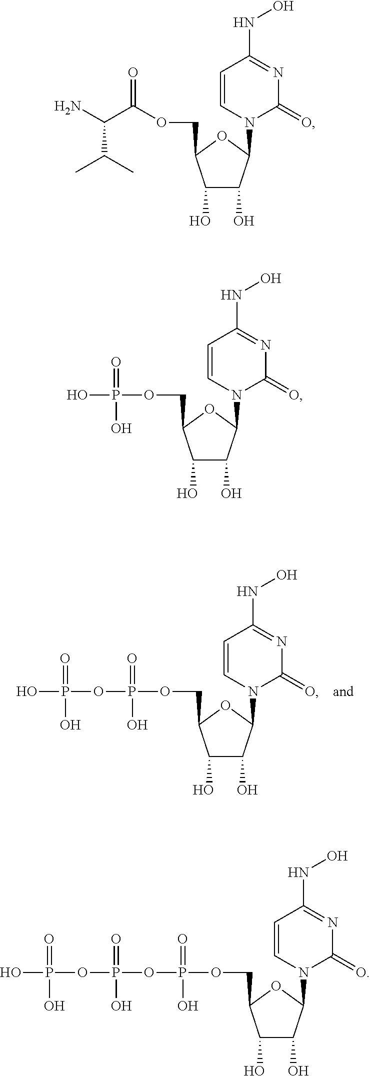 [SMILES: CC(C)[C@H](N)C(=O)OC[C@H]1O[C@@H](n2ccc(NO)nc2=O)[C@H](O)[C@@H]1O ; O=c1nc(NO)ccn1[C@@H]1O[C@H](COP(=O)(O)O)[C@@H](O)[C@H]1O ; O=c1nc(NO)ccn1[C@@H]1O[C@H](COP(=O)(O)OP(=O)(O)O)[C@@H](O)[C@H]1O ; O=c1nc(NO)ccn1[C@@H]1O[C@H](COP(=O)(O)OP(=O)(O)OP(=O)(O)O)[C@@H](O)[C@H]1O]

In exemplified embodiments,

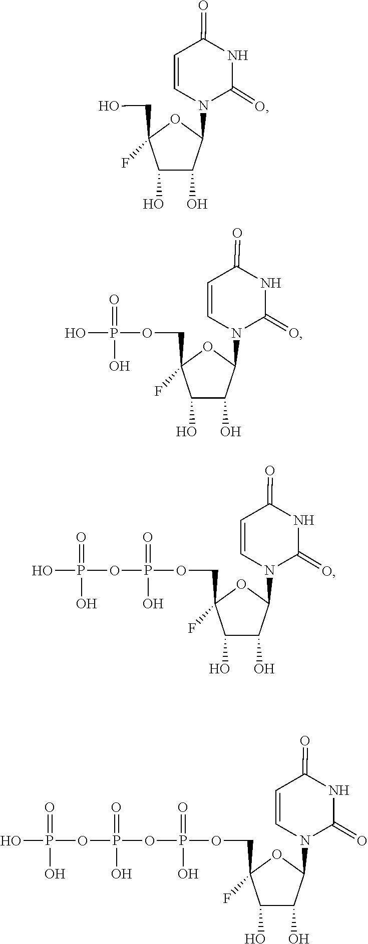 [SMILES: O=c1ccn([C@@H]2O[C@](F)(CO)[C@@H](O)[C@H]2O)c(=O)[nH]1 ; O=c1ccn([C@@H]2O[C@](F)(COP(=O)(O)O)[C@@H](O)[C@H]2O)c(=O)[nH]1 ; O=c1ccn([C@@H]2O[C@](F)(COP(=O)(O)OP(=O)(O)O)[C@@H](O)[C@H]2O)c(=O)[nH]1 ; O=c1ccn([C@@H]2O[C@](F)(COP(=O)(O)OP(=O)(O)OP(=O)(O)O)[C@@H](O)[C@H]2O)c(=O)[nH]1]

or a pharmaceutical or physiological salt thereof with

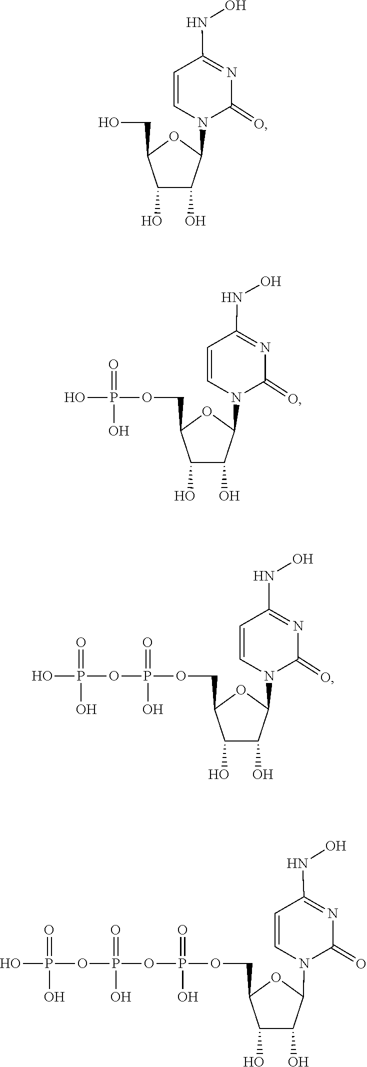 [SMILES: O=c1nc(NO)ccn1[C@@H]1O[C@H](CO)[C@@H](O)[C@H]1O ; O=c1nc(NO)ccn1[C@@H]1O[C@H](COP(=O)(O)O)[C@@H](O)[C@H]1O ; O=c1nc(NO)ccn1[C@@H]1O[C@H](COP(=O)(O)OP(=O)(O)O)[C@@H](O)[C@H]1O ; O=c1nc(NO)ccn1C1O[C@H](COP(=O)(O)OP(=O)(O)OP(=O)(O)O)[C@@H](O)[C@H]1O]

or a pharmaceutical or physiological salt thereof can be found in combination in host cells, tissues, and/or organs that are and are not infected with a virus.

In exemplified embodiments,

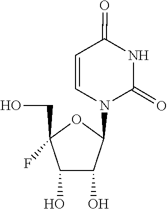 [SMILES: O=c1ccn([C@@H]2O[C@](F)(CO)[C@@H](O)[C@H]2O)c(=O)[nH]1]

or a pharmaceutical or physiological salt thereof can be found in combination with

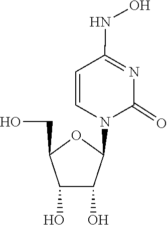 [SMILES: O=c1nc(NO)ccn1[C@@H]1O[C@H](CO)[C@@H](O)[C@H]1O]

or a pharmaceutical or physiological salt thereof in host plasma or whole blood.

In exemplified embodiments,

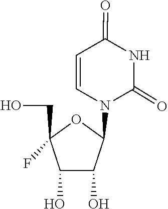 [SMILES: O=c1ccn([C@@H]2O[C@](F)(CO)[C@@H](O)[C@H]2O)c(=O)[nH]1]

or a pharmaceutical or physiological salt thereof can be found in combination with

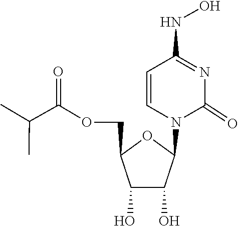

or a pharmaceutical or physiological salt thereof in host plasma or whole blood.

In exemplified embodiments,

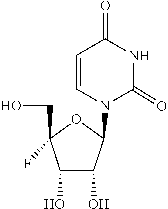 [SMILES: O=c1ccn([C@@H]2O[C@](F)(CO)[C@@H](O)[C@H]2O)c(=O)[nH]1]

or a pharmaceutical or physiological salt thereof can be found in combination with

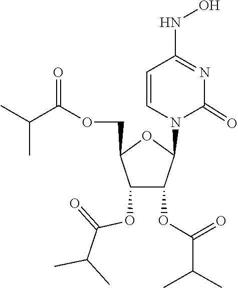 [SMILES: CC(C)C(=O)OC[C@H]1O[C@@H](n2ccc(NO)nc2=O)[C@H](OC(=O)C(C)C)[C@@H]1OC(=O)C(C)C]

or a pharmaceutical or physiological salt thereof in host plasma or whole blood.

In exemplified embodiments,

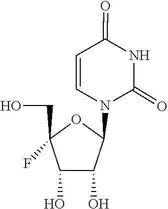 [SMILES: O=c1ccn([C@@H]2O[C@](F)(CO)[C@@H](O)[C@H]2O)c(=O)[nH]1]

or a pharmaceutical or physiological salt thereof can be found in combination with

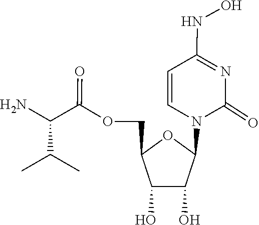 [SMILES: CC(C)[C@H](N)C(=O)OC[C@H]1O[C@@H](n2ccc(NO)nc2=O)[C@H](O)[C@@H]1O]

or a pharmaceutical or physiological salt thereof in host plasma or whole blood.

In exemplified embodiments,

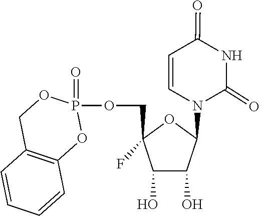 [SMILES: O=c1ccn([C@@H]2O[C@](F)(COP3(=O)OCc4ccccc4O3)[C@@H](O)[C@H]2O)c(=O)[nH]1]

or a pharmaceutical or physiological salt thereof can be found in combination with

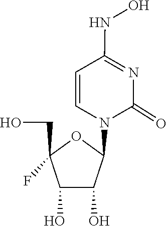 [SMILES: O=c1nc(NO)ccn1[C@@H]1O[C@](F)(CO)[C@@H](O)[C@H]1O]

or a pharmaceutical or physiological salt thereof in host plasma or whole blood.

In exemplified embodiments,

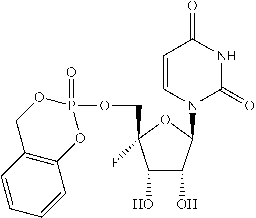 [SMILES: O=c1ccn([C@@H]2O[C@](F)(COP3(=O)OCc4ccccc4O3)[C@@H](O)[C@H]2O)c(=O)[nH]1]

or a pharmaceutical or physiological salt thereof can be found in combination with

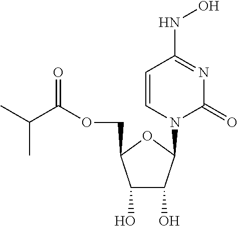 [SMILES: CC(C)C(=O)OC[C@H]1O[C@@H](n2ccc(NO)nc2=O)[C@H](O)[C@@H]1O]

or a pharmaceutical or physiological salt thereof in host plasma or whole blood.

In exemplified embodiments,

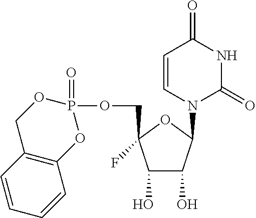 [SMILES: O=c1ccn([C@@H]2O[C@](F)(COP3(=O)OCc4ccccc4O3)[C@@H](O)[C@H]2O)c(=O)[nH]1]

or a pharmaceutical or physiological salt thereof can be found in combination with

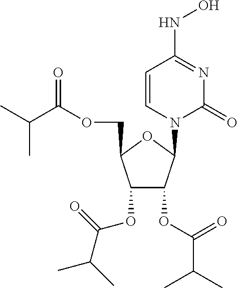 [SMILES: CC(C)C(=O)OC[C@H]1O[C@@H](n2ccc(NO)nc2=O)[C@H](OC(=O)C(C)C)[C@@H]1OC(=O)C(C)C]

or a pharmaceutical or physiological salt thereof in host plasma or whole blood.

In exemplified embodiments,

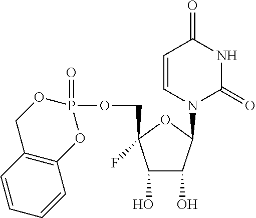 [SMILES: O=c1ccn([C@@H]2O[C@](F)(COP3(=O)OCc4ccccc4O3)[C@@H](O)[C@H]2O)c(=O)[nH]1]

or a pharmaceutical or physiological salt thereof can be found in combination with

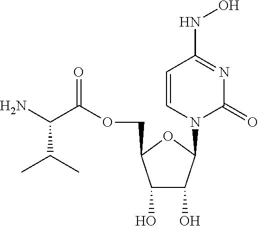 [SMILES: CC(C)[C@H](N)C(=O)OC[C@H]1O[C@@H](n2ccc(NO)nc2=O)[C@H](O)[C@@H]1O]

or a pharmaceutical or physiological salt thereof in host plasma or whole blood.

In yet another aspect, the at least two direct acting antiviral agents comprises a drug combination selected from the group consisting of: a compound of this invention, with one or more of ABT-450 and/or ABT-267, and/or ABT-333, and/or ABT-493, and/or ABT-530; a novel compound of this invention with a compound disclosed in any of US 2010/0144608; U.S. 61/339,964; US 2011/0312973; WO 2009/039127; US 2010/0317568; 2012/151158; US 2012/0172290; WO 2012/092411; WO 2012/087833; WO 2012/083170; WO 2009/039135; US 2012/0115918; WO 2012/051361; WO 2012/009699; WO 2011/156337; US 2011/0207699; WO 2010/075376; US 7,9105,95; WO 2010/120935; WO 2010/111437; WO 2010/111436; US 2010/0168384 or US 2004/0167123; a compound of this invention with one or more of Simeprevir, and/or GSK805; a compound of this invention with one or more of Asunaprevir, and/or Daclastavir, and/or BMS-325; a compound of this invention with one or more of GS-9451, and/or Ledisasvir and/or Sofosbuvir, and/or GS-9669; a compound of this invention with one or more of ACH-2684, and/or ACH-3102, and/or ACH-3422; a compound of this invention with one or more of Boceprevir, and/or MK-8742; a compound of this invention with one or more of Faldaprevir and/or Deleobuvir; a compound of this invention with PPI-668; a compound of this invention with one or more of telaprevir and/or VX-135; a compound of this invention with one or more of Samatasvir and/or IDX-437; a compound of this invention with PSI-7977 and/or PSI-938, a compound of this invention with BMS-790052 and/or BMS-650032; a compound of this invention with GS-5885 and/or GS-9451; a compound of this invention with GS-5885, GS-9190 and/or GS-9451; a compound of this invention in combination with BI-201335 and/or BI-27127; a compound of this invention in combination with telaprevir and/or VX-222; a compound of this invention combination with PSI-7977 and/or TMC-435; and a compound of this invention in combination with danoprevir and/or R7128.

In certain embodiments, pharmaceutical compositions disclosed herein can be administered in an effective amount to a patient in need thereof greater than or equal to 3, 4, 5, 6, 7, 8, 9, 10, 11, 12, 13, 14, 15, 16, 17, 18, 19, or 20 days after clinical signs of disease are observed.

In certain embodiments, pharmaceutical compositions disclosed herein can be administered in an effective amount to a patient in need thereof resulting in 20%, 25%, 30%, 35%, 40%, 45%, 50%, 55%, 60%, 65%, 70%, 75%, 80%, 85%, 90%, 95%, and 100% protection of a population. Such protection of the population can be obtained when administered in an effective amount to a patient in need thereof greater than or equal to 3, 4, 5, 6, 7, 8, 9, 10, 11, 12, 13, 14, 15, 16, 17, 18, 19, 20 days after clinical signs of disease are observed.

In another embodiment, the compounds provided herein can treat encephalitic viral infection. Encephalitic viral infection can be treated by the compounds delivery to the brain. Delivery to the brain can occur by administration to the patient by any of the means described herein, including for example oral, subcutaneous, i.v., i.m., etc. which results in compound concentrations in the brain sufficient to treat infection. In a particular embodiment, EIDD-2749,

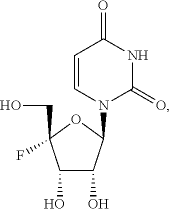 [SMILES: O=c1ccn([C@@H]2O[C@](F)(CO)[C@@H](O)[C@H]2O)c(=O)[nH]1]

and prodrugs thereof can result in concentrations in the brain sufficient to treat encephalitic infection in the brain, for example by arenavirus infection including more specifically Lassa fever virus, Junin virus, lymphocytic choriomeningitis virus, Guanarito virus, Lujo virus, Machupo virus, Sabia virus, and Whitewater Arroyo virus. The compounds of the invention, for example EIDD-2749 and its prodrugs, can also be administered to a post-symptomatic patient to treat any residual viral infection in the brain and central nervous system.

In one aspect of the disclosure, an “infection” or “bacterial infection” refers to an infection caused by acinetobacter spp, bacteroides spp, burkholderia spp, campylobacter spp, chlamydia spp, chlamydophila spp, clostridium spp, enterobacter spp, enterococcus spp, escherichia spp, fusobacterium spp, gardnerella spp, haemophilus spp, helicobacter spp, klebsiella spp, legionella spp, moraxella spp, morganella spp, mycoplasma spp, neisseria spp, peptococcus spp peptostreptococcus spp, proteus spp, pseudomonas spp, salmonella spp, serratia spp., staphylococcus spp, streptoccocus spp, stenotrophomonas spp, or ureaplasma spp.

In one aspect of the disclosure, an “infection” or “bacterial infection” refers to an infection caused by Acinetobacter baumanii, Acinetobacter haemolyticus, Acinetobacter junii, Acinetobacter johnsonii, Acinetobacter iwoffi, Bacteroides bivius, Bacteroides fragilis, Burkholderia cepacia, Campylobacter jejuni, Chlamydia pneumoniae, Chlamydia urealyticus, Chlamydophila pneumoniae, Clostridium difficile, Enterobacter aerogenes, Enterobacter cloacae, Enterococcus faecalis, Enterococcus faecium, Escherichia coli, Gardnerella vaginalis, Haemophilus par influenzae, Haemophilus influenzae, Helicobacter pylori, Klebsiella pneumoniae, Legionella pneumophila, methicillin-resistant Staphylococcus aureus, methicillin-susceptible Staphylococcus aureus, Moraxella catarrhalis, Morganella morganii, Mycoplasma pneumoniae, Neisseria gonorrhoeae, penicillin-resistant Streptococcus pneumoniae, penicillin-susceptible Streptococcus pneumoniae, Peptostreptococcus magnus, Peptostreptococcus micros, Peptostreptococcus anaerobius, Peptostreptococcus asaccharolyticus, Peptostreptococcus prevotii, Peptostreptococcus tetradius, Peptostreptococcus vaginalis, Proteus mirabilis, Pseudomonas aeruginosa, quino lone-resistant Staphylococcus aureus, quinolone-resistant Staphylococcus epidermis, Salmonella typhi, Salmonella paratyphi, Salmonella enteritidis, Salmonella typhimurium, Serratia marcescens, Staphylococcus aureus, Staphylococcus epidermidis, Staphylococcus saprophyticus, Streptoccocus agalactiae, Streptococcus pneumoniae, Streptococcus pyogenes, Stenotrophomonas maltophilia, Ureaplasma urealyticum, vancomycin-resistant Enterococcus faecium, Vancomycin-resistant Enterococcus faecalis, vancomycin-resistant Staphylococcus aureus, vancomycin-resistant Staphylococcus epidermis, Mycobacterium tuberculosis, Clostridium perfringens, Klebsiella oxytoca, Neisseria miningitidis, Proteus vulgaris, or coagulase-negative Staphylococcus (including Staphylococcus lugdunensis, Staphylococcus capitis, Staphylococcus hominis, or Staphylococcus saprophytic).

In one aspect of the disclosure “infection” or “bacterial infection” refers to aerobes, obligate anaerobes, facultative anaerobes, gram-positive bacteria, gram-negative bacteria, gram-variable bacteria, or atypical respiratory pathogens.

In some embodiments, the disclosure relates to treating a bacterial infection such as a gynecological infection, a respiratory tract infection (RTI), a sexually transmitted disease, or a urinary tract infection.

In some embodiments, the disclosure relates to treating a bacterial infection such as an infection caused by drug resistant bacteria.

In some embodiments, the disclosure relates to treating a bacterial infection such as community-acquired pneumoniae, hospital-acquired pneumoniae, skin & skin structure infections, gonococcal cervicitis, gonococcal urethritis, febrile neutropenia, osteomyelitis, endocarditis, urinary tract infections and infections caused by drug resistant bacteria such as penicillin-resistant Streptococcus pneumoniae, methicillin-resistant Staphylococcus aureus, methicillin-resistant Staphylococcus epidermidis and vancomycin-resistant enterococci, syphilis, ventilator-associated pneumonia, intra-abdominal infections, gonorrhoeae, meningitis, tetanus, or tuberculosis.

In some embodiments, the disclosure relates to treating a fungal infections such as infections caused by tinea versicolor, microsporum, trichophyton, epidermophyton, candidiasis, cryptococcosis, or aspergillosis.

In some embodiments, the disclosure relates to treating an infection caused by protozoa including, but not limited to, malaria, amoebiasis, giardiasis, toxoplasmosis, cryptosporidiosis, trichomoniasis, leishmaniasis, sleeping sickness, or dysentery.

Certain compounds disclosed herein are useful to prevent or treat an infection of a malarial parasite in a subject and/or for preventing, treating and/or alleviating complications and/or symptoms associated therewith and can then be used in the preparation of a medicament for the treatment and/or prevention of such disease. The malaria may be caused by Plasmodium falciparum, P. vivax, P. ovale, or P. malariae.

In one embodiment, the compound is administered after the subject has been exposed to the malaria parasite. In another embodiment, a compound disclosed herein is administered before the subject travels to a country where malaria is endemic.

The compounds or the above-mentioned pharmaceutical compositions may also be used in combination with one or more other therapeutically useful substances selected from the group comprising antimalarials like quinolines (e.g., quinine, chloroquine, amodiaquine, mefloquine, primaquine, tafenoquine); peroxide antimalarials (e.g., artemisinin, artemether, artesunate); pyrimethamine-sulfadoxine antimalarials (e.g., Fansidar); hydroxynaphtoquinones (e.g., atovaquone); acroline-type antimalarials (e.g., pyronaridine); and antiprotozoal agents such as ethylstibamine, hydroxystilbamidine, pentamidine, stilbamidine, quinapyramine, puromycine, propamidine, nifurtimox, melarsoprol, nimorazole, nifuroxime, aminitrozole and the like.

In an embodiment, compounds disclosed herein can be used in combination one additional drug selected from the group consisting of chloroquine, artemesin, qinghaosu, 8-aminoquinoline, amodiaquine, arteether, artemether, artemisinin, artesunate, artesunic acid, artelinic acid, atovoquone, azithromycine, biguanide, chloroquine phosphate, chlorproguanil, cycloguanil, dapsone, desbutyl halofantrine, desipramine, doxycycline, dihydrofolate reductase inhibitors, dipyridamole, halofantrine, haloperidol, hydroxychloroquine sulfate, imipramine, mefloquine, penfluridol, phospholipid inhibitors, primaquine, proguanil, pyrimethamine, pyronaridine, quinine, quinidine, quinacrineartemisinin, sulfonamides, sulfones, sulfadoxine, sulfalene, tafenoquine, tetracycline, tetrandine, triazine, salts or mixture thereof.

### Cancer

In a typical embodiment, the disclosure relates to a method treating cancer comprising administering to a patient a compound disclosed herein. In some embodiments, the disclosure relates to a compound disclosed herein, or a pharmaceutically acceptable salt thereof for uses in treating cancer.

In some embodiments, the disclosure relates to a compound disclosed herein, or a pharmaceutically acceptable salt thereof, as defined herein for use in the treatment of cancer of the breast, colorectum, lung (including small cell lung cancer, non-small cell lung cancer and bronchioalveolar cancer) and prostate.

In some embodiments, the disclosure relates to a compound disclosed herein, or a pharmaceutically acceptable salt thereof, as defined herein for use in the treatment of cancer of the bile duct, bone, bladder, head and neck, kidney, liver, gastrointestinal tissue, oesophagus, ovary, endometrium, pancreas, skin, testes, thyroid, uterus, cervix and vulva, and of leukaemias (including ALL and CML), multiple myeloma and lymphomas.

In some embodiments, the disclosure relates to a compound disclosed herein, or a pharmaceutically acceptable salt thereof, as defined herein for use in the treatment of lung cancer, prostate cancer, melanoma, ovarian cancer, breast cancer, endometrial cancer, kidney cancer, gastric cancer, sarcomas, head and neck cancers, tumors of the central nervous system and their metastases, and also for the treatment of glioblastomas.

In some embodiments, compounds disclosed herein could be used in the clinic either as a single agent by itself or in combination with other clinically relevant agents. This compound could also prevent the potential cancer resistance mechanisms that may arise due to mutations in a set of genes.

The anti-cancer treatment defined herein may be applied as a sole therapy or may involve, in addition to the compound of the disclosure, conventional surgery or radiotherapy or chemotherapy. Such chemotherapy may include one or more of the following categories of anti-tumour agents:
(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulfan and nitrosoureas); antimetabolites (for example antifolates such as fluoropyrimidines like 5-fluorouracil and gemcitabine, tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin); and proteosome inhibitors (for example bortezomib [Velcade®]); and the agent anegrilide [Agrylin®]; and the agent alpha-interferon;
(ii) cytostatic agents such as anti-estrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of Su-reductase such as finasteride;
(iii) agents that inhibit cancer cell invasion (for example metalloproteinase inhibitors like marimastat and inhibitors of urokinase plasminogen activator receptor function); (iv) inhibitors of growth factor function, for example such inhibitors include growth factor antibodies, growth factor receptor antibodies (for example the anti-erbb2 antibody trastuzumab [Herceptin™] and the anti-erbbl antibody cetuximab), farnesyl transferase inhibitors, tyrosine kinase inhibitors and serine/threonine kinase inhibitors, for example inhibitors of the epidermal growth factor family (for example EGFR family tyrosine kinase inhibitors such as: N-(3-chloro-4-fluorophenyl)-7-methoxy-6-(3-morpholinopropoxy)quinazolin-4-a mine (gefitinib), N-(3-ethynylphenyl)-6,7-bis(2-methoxyethoxy)quinazolin-4-amine (erlotinib), and 6-acrylamido-N-(3-chloro-4-fluorophenyl)-7-(3-morpholinopropoxy)quinazolin-4-amine (CI 1033), for example inhibitors of the platelet-derived growth factor family and for example inhibitors of the hepatocyte growth factor family, for example inhibitors or phosphotidylinositol 3-kinase (PI3K) and for example inhibitors of mitogen activated protein kinase kinase (MEK1/2) and for example inhibitors of protein kinase B (PKB/Akt), for example inhibitors of Src tyrosine kinase family and/or Abelson (AbI) tyrosine kinase family such as dasatinib (BMS-354825) and imatinib mesylate (Gleevec™); and any agents that modify STAT signalling;
(v) antiangiogenic agents such as those which inhibit the effects of vascular endothelial growth factor, (for example the anti-vascular endothelial cell growth factor antibody bevacizumab [Avastin™]) and compounds that work by other mechanisms (for example linomide, inhibitors of integrin ocvP3 function and angiostatin);
(vi) vascular damaging agents such as Combretastatin A4;
(vii) antisense therapies, for example those which are directed to the targets listed above, such as an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies, and approaches using the immunomodulatory drugs thalidomide and lenalidomide [Revlimid®].

Such conjoint treatment may be achieved by way of the simultaneous, sequential or separate dosing of the individual components of the treatment. Such combination products employ the compounds of this disclosure, or pharmaceutically acceptable salts thereof, within the dosage range described hereinbefore and the other pharmaceutically-active agent within its approved dosage range.

### Formulations

Pharmaceutical compositions disclosed herein may be in the form of pharmaceutically acceptable salts, as generally described below. Some preferred, but non-limiting examples of suitable pharmaceutically acceptable organic and/or inorganic acids are hydrochloric acid, hydrobromic acid, sulfuric acid, nitric acid, acetic acid and citric acid, as well as other pharmaceutically acceptable acids known per se (for which reference is made to the references referred to below).

When the compounds of the disclosure contain an acidic group as well as a basic group, the compounds of the disclosure may also form internal salts, and such compounds are within the scope of the disclosure. When a compound of the disclosure contains a hydrogen-donating heteroatom (e.g., NH), the disclosure also covers salts and/or isomers formed by the transfer of the hydrogen atom to a basic group or atom within the molecule.

Pharmaceutically acceptable salts of the compounds include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases that form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds described herein may be administered in the form of prodrugs. A prodrug can include a covalently bonded carrier that releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a mammalian subject, cleaves to form a free hydroxyl group. Examples of prodrugs include, but are not limited to, acetate, formate and benzoate derivatives of alcohol functional groups in the compounds. Methods of structuring a compound as a prodrug are known, for example, in Testa and Mayer, Hydrolysis in Drug and Prodrug Metabolism, Wiley (2006).

Typical prodrugs form the active metabolite by transformation of the prodrug by hydrolytic enzymes, the hydrolysis of amide, lactams, peptides, carboxylic acid esters, epoxides or the cleavage of esters of inorganic acids. It has been shown that ester prodrugs are readily degraded in the body to release the corresponding alcohol. See e.g., Imai, Drug Metab Pharmacokinet. (2006) 21(3):173-85, entitled “Human carboxylesterase isozymes: catalytic properties and rational drug design.”

Pharmaceutical compositions for use in the present disclosure typically comprise an effective amount of a compound and a suitable pharmaceutical acceptable carrier. The preparations may be prepared in a manner known per se, which usually involves mixing the at least one compound according to the disclosure with the one or more pharmaceutically acceptable carriers, and, if desired, in combination with other pharmaceutical active compounds, when necessary under aseptic conditions. Reference is made to U.S. Pat. Nos. 6,372,778, 6,369,086, 6,369,087 and 6,372,733 and the further references mentioned above, as well as to the standard handbooks, such as the latest edition of Remington's Pharmaceutical Sciences.

Generally, for pharmaceutical use, the compounds may be formulated as a pharmaceutical preparation comprising at least one compound and at least one pharmaceutically acceptable carrier, diluent or excipient, and optionally one or more further pharmaceutically active compounds.

The pharmaceutical preparations of the disclosure are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the disclosure, e.g., about 10, 25, 50, 100, 200, 300 or 400 mg per unit dosage.

The compounds can be administered by a variety of routes including the oral, ocular, rectal, transdermal, subcutaneous, sublingual, intravenous, intramuscular or intranasal routes, depending mainly on the specific preparation used. The compound will generally be administered in an “effective amount”, by which is meant any amount of a compound that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram body weight of the patient per day, every other day, twice weekly, or weekly, more often between 0.1 and 500 mg, such as between 1 and 250 mg, for example about 5, 10, 20, 50, 100, 150, 200 or 250 mg, per kilogram body weight of the patient per day, every other day, twice weekly, or weekly, which may be administered as a single daily, every other day, twice weekly, or weekly dose, or divided over one or more daily, every other day, twice weekly, or weekly doses. The amount(s) to be administered, the route of administration and the further treatment regimen may be determined by the treating clinician, depending on factors such as the age, gender and general condition of the patient and the nature and severity of the disease/symptoms to be treated. Reference is made to U.S. Pat. Nos. 6,372,778, 6,369,086, 6,369,087 and 6,372,733 and the further references mentioned above, as well as to the standard handbooks, such as the latest edition of Remington's Pharmaceutical Sciences.

For an oral administration form, the compound can be mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, cornstarch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, the compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compounds of the disclosure or their physiologically tolerable salts in a pharmaceutically acceptable solvent, such as ethanol or water, or a mixture of such solvents. If required, the formulation may additionally contain other pharmaceutical auxiliaries such as surfactants, emulsifiers and stabilizers as well as a propellant.

For subcutaneous or intravenous administration, the compounds, if desired with the substances customary therefore such as solubilizers, emulsifiers or further auxiliaries are brought into solution, suspension, or emulsion. The compounds may also be lyophilized and the lyophilizates obtained used, for example, for the production of injection or infusion preparations. Suitable solvents are, for example, water, physiological saline solution or alcohols, e.g. ethanol, propanol, glycerol, sugar solutions such as glucose or mannitol solutions, or mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, the formulations may be prepared by mixing the compounds of formula I with a suitable non-irritating excipient, such as cocoa butter, synthetic glyceride esters or polyethylene glycols, which are solid at ordinary temperatures, but liquefy and/or dissolve in the rectal cavity to release the drug.

In certain embodiments, it is contemplated that these compositions can be extended release formulations. Typical extended release formations utilize an enteric coating. Typically, a barrier is applied to oral medication that controls the location in the digestive system where it is absorbed. Enteric coatings prevent release of medication before it reaches the small intestine. Enteric coatings may contain polymers of polysaccharides, such as maltodextrin, xanthan, scleroglucan dextran, starch, alginates, pullulan, hyaloronic acid, chitin, chitosan and the like; other natural polymers, such as proteins (albumin, gelatin etc.), poly-L-lysine; sodium poly(acrylic acid); poly(hydroxyalkylmethacrylates) (for example poly(hydroxyethylmethacrylate)); carboxypolymethylene (for example Carbopol™); carbomer; polyvinylpyrrolidone; gums, such as guar gum, gum arabic, gum karaya, gum ghatti, locust bean gum, tamarind gum, gellan gum, gum tragacanth, agar, pectin, gluten and the like; poly(vinyl alcohol); ethylene vinyl alcohol; polyethylene glycol (PEG); and cellulose ethers, such as hydroxymethylcellulose (HMC), hydroxyethylcellulose (HEC), hydroxypropylcellulose (HPC), methylcellulose (MC), ethylcellulose (EC), carboxyethylcellulose (CEC), ethylhydroxyethylcellulose (EHEC), carboxymethylhydroxyethylcellulose (CMHEC), hydroxypropylmethyl-cellulose (HPMC), hydroxypropylethylcellulose (HPEC) and sodium carboxymethylcellulose (Na-CMC); as well as copolymers and/or (simple) mixtures of any of the above polymers. Certain of the above-mentioned polymers may further be crosslinked by way of standard techniques.

The choice of polymer will be determined by the nature of the active ingredient/drug that is employed in the composition of the disclosure as well as the desired rate of release. In particular, it will be appreciated by the skilled person, for example in the case of HPMC, that a higher molecular weight will, in general, provide a slower rate of release of drug from the composition. Furthermore, in the case of HPMC, different degrees of substitution of methoxyl groups and hydroxypropoxyl groups will give rise to changes in the rate of release of drug from the composition. In this respect, and as stated above, it may be desirable to provide compositions of the disclosure in the form of coatings in which the polymer carrier is provided by way of a blend of two or more polymers of, for example, different molecular weights in order to produce a particular required or desired release profile.

Microspheres of polylactide, polyglycolide, and their copolymers poly(lactide-co-glycolide) may be used to form sustained-release protein delivery systems. Proteins can be entrapped in the poly(lactide-co-glycolide) microsphere depot by a number of methods, including formation of a water-in-oil emulsion with water-borne protein and organic solvent-borne polymer (emulsion method), formation of a solid-in-oil suspension with solid protein dispersed in a solvent-based polymer solution (suspension method), or by dissolving the protein in a solvent-based polymer solution (dissolution method). One can attach poly(ethylene glycol) to proteins (PEGylation) to increase the in vivo half-life of circulating therapeutic proteins and decrease the chance of an immune response.

Liposomal suspensions (including liposomes targeted to viral antigens) may also be prepared by conventional methods to produce pharmaceutically acceptable carriers. This may be appropriate for the delivery of free nucleosides, acyl nucleosides or phosphate ester prodrug forms of the nucleoside compounds according to the present invention.

It is appreciated that nucleosides of the present invention have several chiral centers and may exist in and be isolated in optically active and racemic forms. Some compounds may exhibit polymorphism. It is to be understood that the present invention encompasses any racemic, optically active, diastereomeric, polymorphic, or stereoisomeric form, or mixtures thereof, of a compound of the invention, which possess the useful properties described herein. It is well known in the art how to prepare optically active forms (for example, by resolution of the racemic form by recrystallization techniques, by synthesis from optically-active starting materials, by chiral synthesis, or by chromatographic separation using a chiral stationary phase).

Carbons of the nucleoside are chiral, their nonhydrogen substituents (the base and the CHOR groups, respectively) can be either cis (on the same side) or trans (on opposite sides) with respect to the sugar ring system. The four optical isomers therefore are represented by the following configurations (when orienting the sugar moiety in a horizontal plane such that the oxygen atom is in the back): cis (with both groups “up”, which corresponds to the configuration of naturally occurring R-D nucleosides), cis (with both groups “down”, which is a nonnaturally occurring R-L configuration), trans (with the C2′ substituent “up” and the C4′ substituent “down”), and trans (with the C2′ substituent “down” and the C4′ substituent “up”). The “D-nucleosides” are cis nucleosides in a natural configuration and the “L-nucleosides” are cis nucleosides in the nonnaturally occurring configuration.

Likewise, most amino acids are chiral (designated as L or D, wherein the L enantiomer is the naturally occurring configuration) and can exist as separate enantiomers.

Examples of methods to obtain optically active materials are known in the art, and include at least the following. i) physical separation of crystals-a technique whereby macroscopic crystals of the individual enantiomers are manually separated. This technique can be used if crystals of the separate enantiomers exist, i.e., the material is a conglomerate, and the crystals are visually distinct; ii) simultaneous crystallization-a technique whereby the individual enantiomers are separately crystallized from a solution of the racemate, possible only if the latter is a conglomerate in the solid state; iii) enzymatic resolutions-a technique whereby partial or complete separation of a racemate by virtue of differing rates of reaction for the enantiomers with an enzyme; iv) enzymatic asymmetric synthesis-a synthetic technique whereby at least one step of the synthesis uses an enzymatic reaction to obtain an enantiomerically pure or enriched synthetic precursor of the desired enantiomer; v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries; vi) diastereomer separations-a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer; vii) first- and second-order asymmetric transformations-a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer; viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions; ix) enantiospecific synthesis from non-racemic precursors—a synthetic technique whereby the desired enantiomer is obtained from non-chiral starting materials and where the stereochemical integrity is not or is only minimally compromised over the course of the synthesis; x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions; xi) chiral gas chromatography-a technique whereby the racemate is volatilized and enantiomers are separated by virtue of their differing interactions in the gaseous mobile phase with a column containing a fixed non-racemic chiral adsorbent phase; xii) extraction with chiral solvents-a technique whereby the enantiomers are separated by virtue of preferential dissolution of one enantiomer into a particular chiral solvent; xiii) transport across chiral membranes-a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through. Chiral chromatography, including simulated moving bed chromatography, is used in one embodiment. A wide variety of chiral stationary phases are commercially available.

Some of the compounds described herein contain olefinic double bonds and unless otherwise specified, are meant to include both E and Z geometric isomers.

In addition, some of the nucleosides described herein, may exist as tautomers, such as, keto-enol tautomers. The individual tautomers as well as mixtures thereof are intended to be encompassed within the compounds of the present invention.

### Combination Therapies

The compound described herein can be administered adjunctively with other active compounds. These compounds include but are not limited to analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics, anti-narcoleptics, and antiviral agents. In a particular embodiment, the antiviral agent is a non-CNS targeting antiviral compound. “Adjunctive administration”, as used herein, means the compound can be administered in the same dosage form or in separate dosage forms with one or more other active agents. The additional active agent(s) can be formulated for immediate release, controlled release, or combinations thereof.

Specific examples of compounds that can be adjunctively administered with the compounds include, but are not limited to, aceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methylsalicylate, methysergid(e), metoclopramide, mianserin, mifepristone, milnacipran, minaprine, mirtazapine, moclobemide, modafinil (an anti-narcoleptic), molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pentazocine, pepsin, perphenazine, phenacetin, phendimetrazine, phenmetrazine, phenylbutazone, phenytoin, phosphatidylserine, pimozide, pirlindole, piroxicam, pizotifen, pizotyline, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone and isomers, salts, and combinations thereof.

In certain embodiments, the exemplary compounds and pharmaceutical compositions can be administered in combination with another antiviral agent(s) such as abacavir, acyclovir, acyclovir, adefovir, amantadine, amprenavir, ampligen, arbidol, AT-511, AT-527, atazanavir, atripla, balapiravir, BCX4430/Galidesivir, boceprevir, cidofovir, combivir, daclatasvir, darunavir, dasabuvir, delavirdine, didanosine, ocosanol, edoxudine, efavirenz, emtricitabine, enfuvirtide, entecavir, famciclovir, favipiravir, fomivirsen, fosamprenavir, foscarnet, fosfonet, ganciclovir, GS-41524, GS-5734/Remdesivir, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, Tenofovir Exalidex, tipranavir, trifluridine, trizivir, tromantadine, truvada, valaciclovir, valganciclovir, vicriviroc, vidarabine, viramidine zalcitabine, zanamivir, or zidovudine and combinations thereof.

In certain embodiments, the exemplary compounds and pharmaceutical compositions can be administered in combination with another agent(s) such as chloroquine, chloroquine phosphate, hydroxychloroquine, hydroxychloroquine sulfate, Ampligen, APNO1, Ganovo, IFX-1, BXT-25, CYNK-001, Tocilizumab, Leronlimab, Ii-key, COVID-19 S-Trimer, Camrelizumab, thymosin, Brilacidin, INO-4800, Prezcobix, cobicistat, mRNA-1273, Arbidol, umifenovir, REGN3048, REGN3051, TNX-1800, fingolimod, methylprednisolone, nitazoxanide, benzopurpin B, C-467929, C-473872, NSC-306711, N-65828, C-21, CGP-42112A, L-163491, xanthoangelol, or bevacizumab and combinations thereof.

In certain embodiments, the exemplary compounds and pharmaceutical compositions disclosed herein can be administered in combination with any of the compounds disclosed in: WO2003090690A2, WO2003090690A3, WO2003090691A2, WO2003090691A3, WO2004005286A2, WO2004005286A3, WO2004006843A2, WO2004006843A3, WO2004031224A2, WO2004031224A3, WO2004035576A2, WO2004035576A3, WO2004035577A2, WO2004035577A3, WO2004050613A2, WO2004050613A3, WO2004064845A1, WO2004064846A1, WO2004096286A2, WO2004096286A3, WO2004096287A2, WO2004096287A3, WO2004096818A2, WO2004096818A3, WO2004100960A2, WO2005002626A2, WO2005002626A3, WO2005012324A2, WO2005012324A3, WO2005028478A1, WO2005039552A2, WO2005039552A3, WO2005042772A1, WO2005042773A1, WO2005047898A2, WO2005047898A3, WO2005063744A2, WO2005063744A3, WO2005063751A1, WO2005064008A1, WO2005064008A9, WO2005066189A1, WO2005070901A2, WO2005070901A3, WO2005072748A1, WO2005117904A2, WO2005117904A3, WO2006015261A2, WO2006015261A3, WO2006017044A2, WO2006017044A3, WO2006020276A2, WO2006020276A3, WO2006033703A1, WO2006047661A2, WO2006047661A3, WO2006069193A2, WO2006069193A3, WO2006091905A1, WO2006110157A2, WO2006110157A3, WO2006110157A9, WO2006125048A2, WO2006125048A3, WO2007009109A2, WO2007009109A3, WO2007011658A1, WO2007014174A2, WO2007014174A3, WO2007014352A2, WO2007014352A3, WO2007079260A1, WO2007079260A9, WO2007126812A2, WO2007126812A3, WO2008003149A2, WO2008003149A3, WO2008005519A2, WO2008005519A3, WO2008005542A2, WO2008005542A3, WO2008005555A1, WO2008009076A2, WO2008009076A3, WO2008009077A2, WO2008009077A3, WO2008009078A2, WO2008009078A3, WO2008009079A2, WO2008009079A3, WO2008010921A2, WO2008010921A3, WO2008011116A2, WO2008011116A3, WO2008011117A2, WO2008011117A3, WO2008013834A1, WO2008016522A2, WO2008016522A3, WO2008077649A1, WO2008077650A1, WO2008077651A1, WO2008100447A2, WO2008100447A3, WO2008103949A1, WO2008133669A2, WO2008133669A3, WO2009005674A2, WO2009005674A3, WO2009005676A2, WO2009005676A3, WO2009005677A2, WO2009005677A3, WO2009005687A1, WO2009005690A2, WO2009005690A3, WO2009005693A1, WO2009006199A1, WO2009006203A1, WO2009009001A1, WO2009009001A9, WO2009088719A1, WO2009105513A2, WO2009105513A3, WO2009132123A1, WO2009132135A1, WO2010002998A1, WO2010005986A1, WO2010011959A1, WO2010075127A1, WO2010077613A1, WO2010080389A1, WO2010093608A1, WO2010132601A1, WO2010151472A1, WO2010151487A1, WO2010151488A1, WO2011005842A1, WO2011011303A1, WO2011031669A1, WO2011031965A1, WO2011035231A1, WO2011049825A1, WO2011079016A1, WO2011088303A1, WO2011088345A1, WO2011106445A1, WO2011143105A1, WO2011143106A1, WO2011146817A1, WO2011150288A1, WO2011156416A1, WO2011156610A2, WO2011156610A3, WO2011156757A1, WO2011163518A1, WO2012003497A1, WO2012003498A1, WO2012012465A1, WO2012012776A1, WO2012037038A1, WO2012039787A1, WO2012039791A1, WO2012068234A2, WO2012068234A3, WO2012068535A1, WO2012078915A1, WO2012087596A1, WO2012088153A1, WO2012088156A1, WO2012088178A1, WO2012138669A1, WO2012138670A1, WO2012142523A2, WO2012142523A3, WO2012145728A1, WO2012151165A1, WO2013006721A1, WO2013006722A1, WO2013006738A1, WO2013010112A1, WO2013025788A1, WO2013040492A2, WO2013040492A3, WO2013066748A1, WO2013075029A1, WO2013082003A1, WO2013090840A1, WO2013090929A1, WO2013096512A1, WO2013096681A1, WO2013103724A1, WO2013103738A1, WO2013106732A1, WO2013115916A1, WO2013116720A1, WO2013116730A1, WO2013138236A1, WO2013158776A1, WO2013159064A1, WO2013173488A1, WO2013173492A1, WO2013185090A1, WO2013185093A1, WO2013185103A1, WO2014008285A1, WO2014028343A1, WO2014055618A1, WO2014070939A1, WO2014074620A1, WO2014100323A1, WO2014100500A1, WO2014110296A1, WO2014110297A1, WO2014110298A1, WO2014134566A2, WO2014134566A3, WO2014145095A1, WO2015023893A1, WO2015069939A1, WO2015084741A2, WO2015084741A3, WO2015099989A1, WO2015100144A1, WO2015108780A1, WO2015120057A1, WO2015130964A1, WO2015130966A1, WO2015179448A1, WO2015191526A2, WO2015191526A3, WO2015191726A1, WO2015191743A1, WO2015191745A1, WO2015191752A1, WO2015191754A2, WO2015191754A3, WO2015196137A1, WO2016007765A1, WO2016018697A1, WO2016028866A1, WO2016033243A1, WO2016033243A9, WO2016036759A1, WO2016096116A1, WO2016096116A1, WO2016105532A1, WO2016105534A1, WO2016105564A1, WO2016106237A1, WO2016141092A1, WO2016161382A1, WO2016168349A1, WO2016186967A1, WO2016205141A1, WO2017004012A1, WO2017004244A1, WO2017035230A1, WO2017048727A1, WO2017049060A1, WO2017059120A1, WO2017059224A2, WO2017059224A3, WO2017083304A1, WO2017106346A2, WO2017106346A3, WO2017106556A1, WO2017184668A1, WO2017184670A2, WO2017184670A3, WO2017205078A1, WO2017205115A1, WO2017223268A1, WO9015065A1, WO9209705A1, WO9307157A1, WO9310820A1, WO9403467A2, WO9403467A3, WO9424144A2, WO9424144A3, WO9507919A1, WO9507920A1, WO9626933A1, WO9817647A1, WO2009114512, WO2014028756, JP5971830, US20160122374, US20170071964, WO2007075145, WO2005021518, WO2007120160, WO2009119167, WO2013049382, WO2018042343, WO2007067515, EP2112164, WO2009128 WO2018115527 963, WO2009128963, WO2008035894, WO2008060331, WO2007044695, CN1911963, CN1903878, WO2006095180, WO2006086561, CN1664100, CN1660912, WO2006051091, WO2006051091, CN1673231, US20060240551, WO2005054469, WO2005060520, US20050106563, US20050069869, WO2005012360, CN1566155, WO2005007671, WO2005058815, WO2017095875, WO200505824, WO2011072487, WO2016180335, WO2004096852, WO2005097165, US20090053173, CN101942026, CN101173275, CN1648249, US20050004063, JP2007043942, WO2005023083, US20060039926, WO2005081716, WO2015081155, WO2010063685, US20070003577, US20060002947, WO2015042373, WO2017070626.

EIDD-1931 and prodrugs thereof, e.g EIDD-2801, can be administered in combination with, or formulated with, another antiviral agent(s) such as:
Nucleoside reverse transcriptase inhibitors (NRTIs)
Non-nucleoside reverse transcriptase inhibitors (NNRTIs)
Protease inhibitors (PIs)
Integrase inhibitors (INSTIs)
Fusion inhibitors (FIs)
Chemokine receptor antagonists
Entry inhibitors

Specific examples of agents include abacavir, acyclovir, acyclovir, adefovir, amantadine, amprenavir, ampligen, arbidol, AT-511, AT-527, atazanavir, atripla, balapiravir, BCX4430/Galidesivir, boceprevir, cidofovir, combivir, daclatasvir, darunavir, dasabuvir, delavirdine, didanosine, docosanol, edoxudine, efavirenz, emtricitabine, enfuvirtide, entecavir, famciclovir, favipiravir, fomivirsen, fosamprenavir, foscarnet, fosfonet, ganciclovir, GS-41524, GS-5734/Remdesivir, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, Tenofovir Exalidex, tipranavir, trifluridine, trizivir, tromantadine, truvada, valaciclovir, valganciclovir, vicriviroc, vidarabine, viramidine zalcitabine, zanamivir, zidovudine, or chloroquine, chloroquine phosphate, hydroxychloroquine, hydroxychloroquine sulfate, Ampligen, APN01, Ganovo, IFX-1, BXT-25, CYNK-001, Tocilizumab, Leronlimab, Ii-key, COVID-19 S-Trimer, Camrelizumab, thymosin, Brilacidin, INO-4800, Prezcobix, cobicistat, mRNA-1273, Arbidol, umifenovir, REGN3048, REGN3051, TNX-1800, fingolimod, methylprednisolone, nitazoxanide, benzopurpin B, C-467929, C-473872, NSC-306711, N-65828, C-21, CGP-42112A, L-163491, xanthoangelol, bevacizumab, polyclonal antibodies derived from patients and monoclonal antibodies (including those antibodies from patients of COVID-19 or monoclonal or polyclonal antibodies which bind SARS-CoV-2), and combinations thereof. In addition, the compounds of this invention can be combined with compounds that are favorable to preventing lung damage associated with COVID-19, including for example anti-IL-6 and TNF inhibitors, specifically including for example, tocilzuma (Actemra), siltuximab (Sylvant), Tocilizumab, Sarilumab, olokizumab (CDP6038), elsimomab, BMS-945429 (ALD518), sirukumab (CNTO 136), levilimab (BCD-089), and CPSI-264 and ALX-0061, ARGX-109, FE301, FM10, infliximab (Remicade), adalimumab (Humira), certolixumab pegol (Cimzia), and golimumab (Simponi), etanercept (Enbrel), Thalidomide (Immunoprin) and its derivatives lenalidomide (Revlimid) and pomalidomide (Pomalyst, Imnovid), xanthine derivatives (e.g. pentoxifylline in) and bupropion and 5-HT, agonist hallucinogens including (R)-DOI, TCB-2, LSD and LA-SS-Az.

In exemplified embodiments, the exemplary compounds and pharmaceutical compositions can be administered in combination with

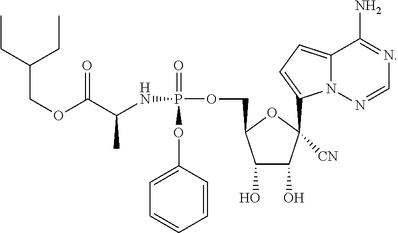 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

In exemplified embodiments,

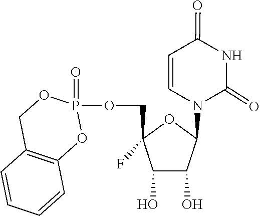 [SMILES: O=c1ccn([C@@H]2O[C@](F)(COP3(=O)OCc4ccccc4O3)[C@@H](O)[C@H]2O)c(=O)[nH]1]

can be administered in comination with

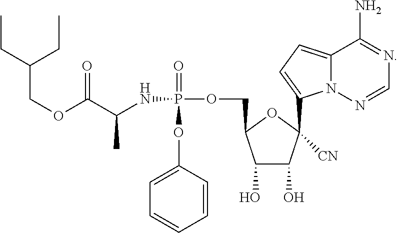 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

In exemplified embodiments,

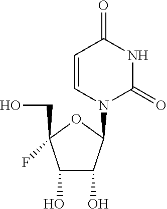 [SMILES: O=c1ccn([C@@H]2O[C@](F)(CO)[C@@H](O)[C@H]2O)c(=O)[nH]1]

can be administered in combination with

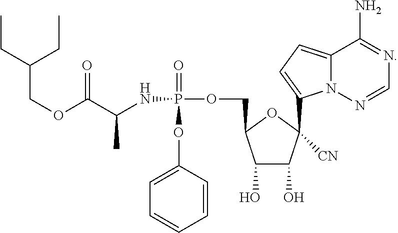 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

In exemplified embodiments,

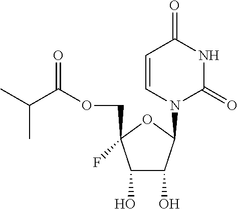 [SMILES: CC(C)C(=O)OC[C@@]1(F)O[C@@H](n2ccc(=O)[nH]c2=O)[C@H](O)[C@@H]1O]

can be administered in commination with

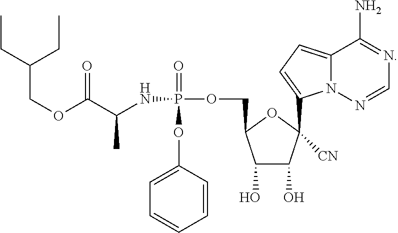 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

### EXAMPLES

### Example 1

### Conjugate Preparation

Mono and diphosphate prodrugs have been prepared by several groups. See Jessen et al., Bioreversible Protection of Nucleoside Diphosphates, Angewandte Chemie-International Edition English 2008, 47 (45), 8719-8722, hereby incorporated by reference.

In order to prevent rupture of the P—O—P anhydride bond, one utilizes a pendant group that fragments rapidly (e.g. bis-(4-acyloxybenzyl)-nucleoside diphosphates (BAB-NDP) that is deacylated by an endogenous esterase) to generate a negative charge on the second phosphate. See also Routledge et al., Synthesis, Bioactivation and Anti-HIV Activity of 4-Acyloxybenzyl-bis(nucleosid-5′-yl) Phosphates, Nucleosides & Nucleotides 1995, 14 (7), 1545-1558 and Meier et al., Comparative study of bis(benzyl)phosphate triesters of 2′,3′-dideoxy-2′,3′-didehydrothymidine (d4T) and cycloSal-d4TMP-hydrolysis, mechanistic insights and anti-HIV activity, Antiviral Chemistry and Chemotherapy 2002, 13, 101-114, both hereby incorporated by reference. Once this occurs, the P—O—P anhydride bond is less susceptible to cleavage and the remaining protecting group can then do its final unraveling to produce the nucleoside diphosphate.

Other methods to prepare diphosphate and monothiodiphosphate prodrugs are shown in FIG. 5. Standard coupling conditions are used to prepare sphingolipid-nucleoside monophosphate prodrugs. The corresponding diphosphate prodrugs may be prepared according to the protocols shown in FIG. 5 and as provided in Smith et al., Substituted Nucleotide Analogs. U.S. Patent Application 2012/0071434; Skowronska et al., Reaction of Oxophosphorane-Sulfenyl and Oxophosphorane-Selenenyl Chlorides with Dialkyl Trimethylsilyl
Phosphites—Novel Synthesis of Compounds Containing a Sulfur or Selenium Bridge Between 2
Phosphoryl Centers, Journal of the Chemical Society-Perkin Transactions 1 1988, 8, 2197-2201;
Dembinski et al., An Expedient Synthesis of Symmetrical Tetra-Alkyl Mono-thiopyrophosphates, Tetrahedron Letters 1994, 35 (34), 6331-6334; Skowronska et al., Novel Synthesis of Symmetrical Tetra-Alkyl Monothiophosphates, Tetrahedron Letters 1987, 28 (36), 4209-4210; and Chojnowski et al., Methods of Synthesis of O, O-Bis TrimethylSilyl Phosphorothiolates. Synthesis-Stuttgart 1977, 10, 683-686, all hereby incorporated by reference in their entirety.

### Example 2

### General Procedure for Base Coupling

The persilylated nucleobase was prepared in a round bottom flask charged with dry nucleobase (15.5 mmol), chlorotrimethylsilane (12.21 mmol), and bis(trimethylsilyl)amine (222 mmol) under nitrogen. The mixture was refluxed with stirring overnight (16 h) until all solids dissolved. The mixture was cooled to room temperature and volatiles were removed by rotary evaporation followed by high vacuum to give persilylated nucleobase. This compound was used immediately in the next step.

The freshly prepared persilylated nucleobase (15.50 mmol) was dissolved in 1,2-dichloroethane (50 mL) or chlorobenzene (50 mL) under nitrogen with stirring at room temperature. A solution of QI-D-ribofuranose 1,2,3,5-tetraacetate (7.75 mmol) in 1,2-dichloroethane (50 mL) or chlorobenzene (50 mL) was added all at once to the stirred mixture.

To this mixture was added SnCl₄ (11.63 mmol) dropwise via syringe, and the mixture was stirred at room temperature 6 h until all starting material was consumed. The mixture was cooled to 0° C. and a sat. aq. NaHCO₃ solution (125 mL) was added. The mixture was warmed to room temperature and stirred 30 min. The mixture was extracted with EtOAc (2×200 mL) and the combined organic layers were washed with brine (1×100 mL), dried over Na₂SO₄, filtered, and concentrated by rotary evaporation to give 5.5 g crude product. The crude material was taken up in dichloromethane, immobilized on Celite, and subjected to flash chromatography to provide the desired acetate protected product. The ribonucleoside was deprotected using the general deprotection conditions.

### Example 3

### General Cytosine Analog Coupling

In a flask charged with N⁴-benzoyl protected cytosine analog (0.793 mmol) was added bis(trimethylsilyl)amine (8.45 mmol) and ammonium sulfate (0.02 mmol) under N2. This was heated at reflux for 2 h, after cooling to rt, solvent was removed in vacuo and further dried under high vacuum for 1 h. The residue was dissolved in dry chlorobenzene (10 ml) and □-D- or D-L-ribofuranose 1,2,3,5-tetraacetate (0.53 mmol) was added. Then SnCl₄ (0.27 ml, 2.3 mmol) was added dropwise. After stirring at rt for 1 h, this was heated to 60° C. overnight.

After cooling to 0° C., solid sodium bicarbonate (0.85 g) was added, followed by EtOAc (5 mL). This was allowed to stir for 15 min and then water (0.5 mL) was added slowly. The insoluble material was filtered off and washed with more EtOAc (2.5 mL). The filtrate was washed with water once, bine once, dried (Na₂SO₄) and concentrated in vacuo. The crude material was purified by SiO₂ column chromatography.

### Example 4

### General Deamination Conditions

A solution of benzoyl protected cytidine ribonucleoside (1.02 mmol) in 80% aqueous AcOH (30 mL) was heated under reflux for 16 h. The solvent was then removed in vacuo and dried under high vacuum. The white solid was triturated with ether, filtered off and washed with more ether to obtain the desired product.

### Example 5

### General Uracil Analog Coupling

The persilylated uracil was prepared in a round bottom flask charged with uracil (15.5 mmol), chlorotrimethylsilane (12.21 mmol), and bis(trimethylsilyl)amine (222 mmol) under nitrogen. The mixture was refluxed with stirring overnight (16 h) until all solids dissolved until a clear colorless solution formed. The mixture was cooled to room temperature and volatiles were removed by rotary evaporation followed by high vacuum to give persilylated uracil. This compound was used immediately in the next step.

The freshly prepared persilylated uracil (15.50 mmol) was dissolved in 1,2-dichloroethane (50 mL) under nitrogen with stirring at room temperature. A solution of □-D- or □-L-ribofuranose 1,2,3,5-tetraacetate (7.75 mmol) in 1,2-dichloroethane (50 mL) was added all at once to the stirred mixture.

To this mixture was added SnCl₄ (11.63 mmol) dropwise via syringe, and the mixture was stirred at room temperature 6 h until all starting material was consumed. The mixture was cooled to 0° C. and a sat. aq. NaHCO₃ solution (125 mL) was added. The mixture was warmed to room temperature and stirred 30 min. The mixture was extracted with EtOAc (2×200 mL) and the combined organic layers were washed with brine (1×100 mL), dried over Na₂SO₄, filtered, and concentrated by rotary evaporation to give 5.5 g crude product. The crude material was taken up in dichloromethane, immobilized on Celite, and subjected to flash chromatography on the Combiflash (120 g column, 5 to 50% EtOAc in hexanes gradient) to provide the product.

### Example 6

### General Acetate or Benzoyl Deprotection Conditions

Benzoyl protected ribonucleoside analog (0.25 mmol) was stirred with 7 N ammonia in MeOH at rt for 15.5 h. The solvent was then removed and the crude material was purified by SiO2 column chromatography to obtain the desired ribonucleoside.

### Example 7

### Synthesis of 1′-Deuterated Nucleoside Analogs

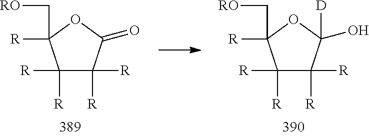

The lactone (0.0325 mol) was added to a dry flask under an argon atmosphere and was then dissolved in dry THE (250 mL). The solution as then cooled to −78° C. and a DIBAL-D solution in toluene (0.065 mol) was dropwise. The reaction was allowed to stir at −78° C. for 3-4 hours. The reaction was then quenched with the slow addition of water (3 mL). The reaction was then allowed to stir while warming to room temperature. The mixture was then diluted with two volumes of diethyl ether and was then poured into an equal volume of saturated sodium potassium tartrate solution. The organic layer was separated, dried over MgSO₄, filtered, and concentrated under reduced pressure. The residue was purified on silica eluting with hexanes/ethyl acetate. The resulting lactol was then converted to an acetate or benzolyate and subjected to base coupling conditions to introduce the desired nucleobase.

### Example 8

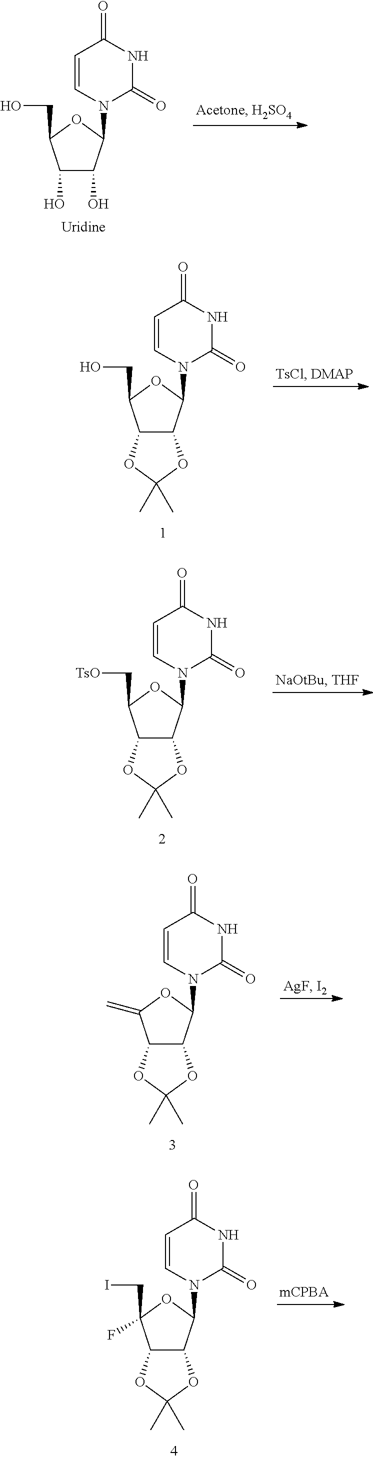 [SMILES: O=c1ccn([C@@H]2O[C@H](CO)[C@@H](O)[C@H]2O)c(=O)[nH]1 ; CC1(C)O[C@@H]2[C@H](O1)[C@@H](CO)O[C@H]2n1ccc(=O)[nH]c1=O ; Cc1ccc(S(=O)(=O)OC[C@H]2O[C@@H](n3ccc(=O)[nH]c3=O)[C@@H]3OC(C)(C)O[C@@H]32)cc1 ; C=C1O[C@@H](n2ccc(=O)[nH]c2=O)[C@@H]2OC(C)(C)O[C@H]12 ; CC1(C)O[C@H]2[C@H](n3ccc(=O)[nH]c3=O)O[C@](F)(CI)[C@H]2O1]

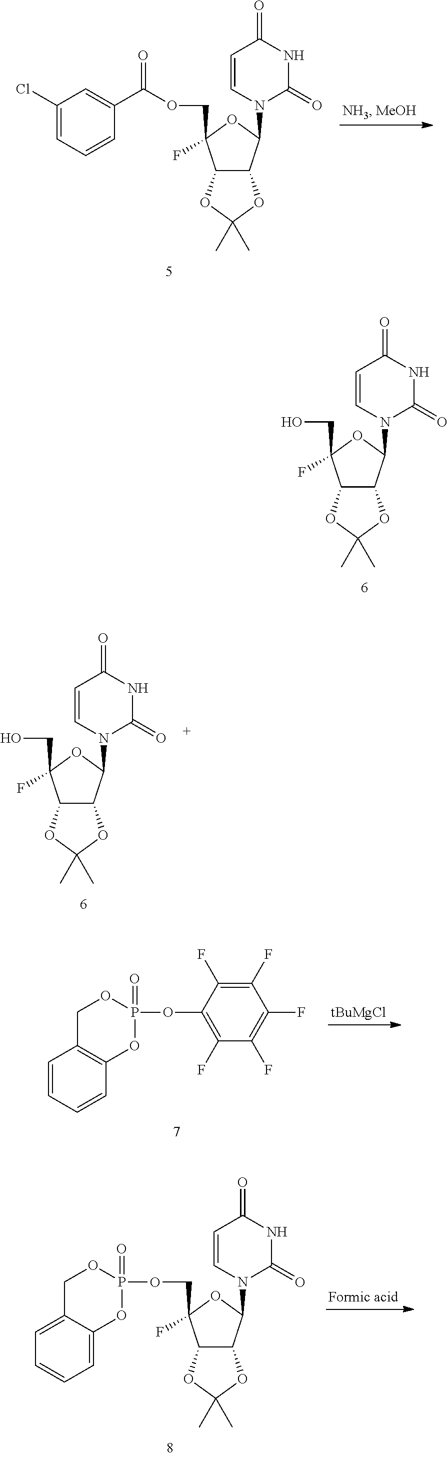

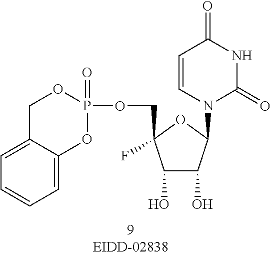 [SMILES: O=c1ccn([C@@H]2O[C@](F)(COP3(=O)OCc4ccccc4O3)[C@@H](O)[C@H]2O)c(=O)[nH]1]

A 1 L rbf was charged with uridine (36.6 g, 150 mmol) and acetone (Volume: 700 ml) with stirring under nitrogen at rt. The slurry was treated with concentrated sulfuric acid (0.800 ml, 15.00 mmol) and the mixture was stirred at rt overnight. After stirring 16 h, triethylamine (41.8 ml, 300 mmol) was added all at once, the mixture was stirred 30 min, and then concentrated by rotary evaporation to give a sticky white solid. The solid was dissolved in boiling iPrOH (−1.4 L) and allowed to cool overnight at rt. After cooling overnight, small crystals had formed. The flask was placed in the freezer for 3 h and more crystals formed. The mixture was vacuum filtered, and the solids were washed with ice-cold iPrOH (2×200 mL) and ice-cold ether (2×200 mL). The solid was recovered to give compound 1 (21.75 g, 77 mmol, 51.0% yield) as a white powdery solid.

A round bottom flask was charged with compound 1 (21.75 g, 77 mmol) and DCM (219 ml) and the mixture was stirred under nitrogen. Solid 4-DMAP (23.37 g, 191 mmol) was added all at once, and the mixture was stirred at rt until all solids dissolved. The mixture was cooled to 0° C., and tosyl chloride (17.50 g, 92 mmol) was added portionwise as a solid over 5 min. The mixture was stirred at rt for 1 h until all starting material was consumed. The mixture was transferred to a separatory funnel, and the organic layer was washed with 1 N HCl (2×200 mL), sat. aq. NaHCO₃ (1×200 mL), and brine (1×200 mL), then dried over Na₂SO₄, filtered and concentrated by rotary evaporation to give compound 2 (34.52 g, 74.8 mmol, 98% yield) as a white solid.

To a stirred solution of compound 2 (3.95 g, 9.01 mmol) in THE (30 mL) at 0° C. under nitrogen. Solid potassium tert-butoxide (3.03 g, 27.0 mmol) was added all at once, the reaction mixture turned into a yellow slurry. The mixture was stirred at 0° C. for 2 h. Silica gel (6 g) and Celite (14 g) were added along with more THF, and the mixture was concentrated by rotary evaporation. Flash chromatography on the Isco (80 g column, 1 to 5% MeOH in DCM) gave compound 3 (2.17 g, 8.15 mmol, 90% yield) as a white powdery solid.

A round bottom flask was charged with a stir bar, compound 3 (2.17 g, 8.15 mmol), silver(I) fluoride (5.17 g, 40.8 mmol), and DCM (Volume: 152 ml, Ratio: 14) at 0° C. To this vigorously stirred mixture was added a solution of iodine (4.14 g, 16.30 mmol) in THE (Volume: 10.87 ml, Ratio: 1.000) dropwise via syringe over 40 min. After addition was complete, the mixture was stirred another 15 min at 0° C., then a 1:1 mixture of sat. aq.

NaHCO₃:sat. aq. Na₂S₂O₃ was added (75 mL) and the whole mixture was filtered through a Celite pad, washing with DCM (2×50 mL). The filtrates were transferred to a separation funnel, and the organic layer was dried over Na₂SO₄, filtered, and concentrated by rotary evaporation to give 4 g. Flash chromatography on the Isco (120 g column, 5 to 25% EtOAc in DCM) gave compound 4 (2.06 g, 5.00 mmol, 61.3% yield) as a pale yellow flaky solid.

A round bottom flask was charged with compound 4 (10.76 g, 26.1 mmol), tetrabutylammonium sulfate (8.86 g, 26.1 mmol), potassium hydrogen phosphate dibasic trihydrate (8.94 g, 39.2 mmol), DCM (Volume: 1088 ml, Ratio: 5) and water (Volume: 218 ml, Ratio: 1.000) and the biphasic mixture was stirred vigorously at rt. To this mixture was added solid mCPBA, 77% w/w (29.3 g, 131 mmol) all at once and the mixture was stirred at rt overnight. After stirring 20 h at rt, all SM had been consumed by TLC analysis. The mixture was quenched by slow addition of sat. aq. Na₂S₂O₃ (375 mL) followed by sat. aq.

Na₂CO₃ (375 mL). The organic layer was removed, and the aqueous layer was extracted with DCM (1×450 mL). The combined organic layers were dried over Na₂SO₄, filtered, and concentrated by rotary evaporation to give 22 g crude. The crude was taken up in DCM, and flash chromatography on the Isco (330 g column, 5 to 25% EtOAc in DCM) gave 10 g of semipure product. The compound was taken up in DCM, and flash chromatography on the Isco (330 g column, 5 to 70% EtOAc in hexanes) gave compound 5 (6.91 g, 15.68 mmol, 60.0% yield) as an off-white flaky solid.

A round bottom flask was charged with compound 5 (3.53 g, 8.0 mmol) and ammonia in MeOH (34.3 ml, 240 mmol) at 0° C. The mixture was stirred for 5 h, at which point all starting material was consumed. The mixture was concentrated by rotary evaporation to give ˜4 g crude as a yellow oil. The crude was taken up in DCM, and flash chromatography on the Isco (120 g column, 1 to 5% MeOH in DCM) gave compound 6 (2.20 g, 7.28 mmol, 91% yield) as a white powdery solid.

A 1 L 3-neck RBF equipped with temperature probe, overhead stirrer and addition funnel (argon inlet) was charged with phosphorus oxychloride (15.50 ml, 166 mmol) in THE (300 ml), evacuated and purged with argon 3×, then cooled to <−70° C. using dry ice/acetone. A solution of 2-(hydroxymethyl)phenol (18.77 g, 151 mmol) and triethylamine (44.3 ml, 317 mmol) in 200 mL of THE was slowly added via addition funnel over 30 minutes. The resulting light tan mixture was slowly warmed to RT and stirred for 3 hrs.

Cooled to 0° C. using an ice bath and added triethylamine (25.3 ml, 181 mmol), then slowly added a THE (100 mL) solution of 2,3,4,5,6-pentafluorophenol (25.05 g, 136 mmol) to the rapidly stirred mixture. Warmed to RT and monitored by TLC (25% EtOAc/hexanes). SM consumed in <2 hrs, only product (Rf=0.5) present. The oil was purified by SGC (glass column, 10-25% EtOAc/hexanes), fractions containing product were pooled and concentrated under reduced pressure to yield compound 7 (41.2 g, 117 mmol, 77% yield) as a white solid.

To a stirred solution of compound 6 (1.95 g, 6.45 mmol) in THE (Volume: 96 ml, Ratio: 5) at 0° C. under nitrogen, was added a solution of tert-butylmagnesium chloride, 1.0 M in THE (14.19 ml, 14.19 mmol) dropwise via syringe. A white precipitate formed; the mixture was warmed to rt and stirred for 30 min, then recooled to 0° C. A solution of compound 7 (5.68 g, 16.13 mmol) in THE (Volume: 19.20 ml, Ratio: 1.000) was added dropwise via syringe, and the mixture was warmed to rt and stirred overnight. After 18 h stirring, a little SM remained and one slightly less polar product had formed. The mixture was quenched by addition of solid NH₄Cl (2 g) and the mixture was immobilized on Celite.

Flash chromatography on the Isco (220 g column, 1 to 5% MeOH in DCM) gave 1.94 g of a white solid that consisted of desired product and pentafluorophenol. The solid was taken up in DCM and washed with sat. aq. NaHCO₃ (3×100 mL). The organic layer was dried over Na₂SO₄, filtered, and concentrated by rotary evaporation to give compound 8 (1.70 g, 3.61 mmol, 56.0% yield) as a white powdery solid.

A round bottom flask was charged with compound 8 (0.250 g, 0.532 mmol) and formic acid, 80% aq. (Volume: 10 mL). The mixture was stirred at rt under nitrogen overnight. After stirring 20 h, all volatiles were removed by rotary evaporation. The residue was taken up in MeOH and immobilized on Celite. Flash chromatography on the Isco (24 g column, 1 to 15% MeOH in DCM) gave a white powdery solid, 175 mg, 90-95% pure by NMR. The white powder was taken up in a 5:1 water:MeCN mixture, and reverse phase flash chromatography on the Isco (100 g C18 column, 100% water to 100% MeCN) gave good separation of the impurity. The fractions containing desired product were concentrated, taken up in 5:1 water:MeCN, frozen in a dry ice bath, and lyophilized to provide compound 9, EIDD-02838.

### Example 9

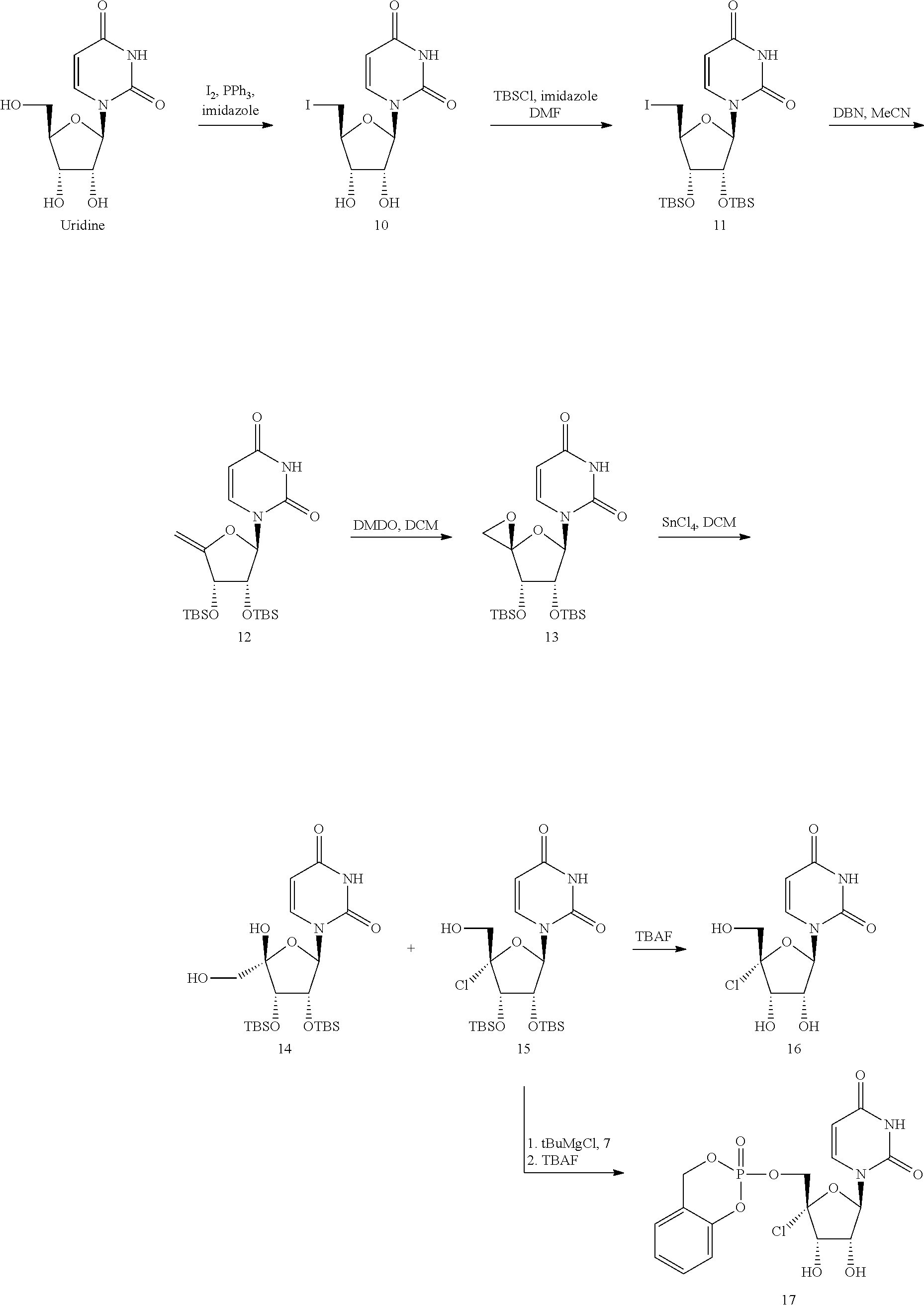

Uridine (1 mmol) was suspended in dioxane (4 mL) followed by the addition of pyridine (2 mmol), PPh₃ (1.5 mmol), and iodine (1.5 mmol) under an argon atmosphere. The mixture was stirred at room temperature overnight. The reaction mixture was quenched with methanol and saturated aqueous Na₂S₂O₃and was then evaporated to dryness to provide crude compound 10, which was used directly in the next step.

Crude compound 10 was dissolved in dry DMF under an argon atmosphere followed by the addition of imidazole (5 equivalents) and TBSCl (4 equivalents) at 0° C. The mixture was allowed to warm to room temperature and stir overnight. The reaction mixture was partitioned between AcOEt/H₂O (3:1). The organic layer was dried over MgSO₄, filtered, and concentrated under reduced pressure. The resulting residue was purified on a silica gel column eluting with hexanes and ethyl acetate to provide compound 11.

Compound 11 was dissolved in dry MeCN and treated with DBN (2.25 equivalents) at 0° C. under an argon atmosphere. The reaction was allowed to stir overnight. The reaction mixture was neutralized with AcOH and then was evaporated to dryness. The residue was partitioned between DCM and saturated aqueous NaHCO₃. The organic layer was dried over MgSO₄, filtered, and concentrated under reduced pressure. The resulting residue was purified on a silica gel column eluting with hexanes and ethyl acetate to provide compound 12.

To a solution of compound 12 in dry DCM (20 mL/mmol 12) was added DMDO (0.1M in acetone, 1.2 equivalents) at −30° C. under an argon atmosphere. The reaction was allowed to stir for 1 hour and was then evaporated to dryness to afford compound 13, which was used immediately in the next step.

To a solution of compound 13 in dry DCM (20 mL/mmol 13) was added SnCl₄ (3 equivalents) at −30° C. under an argon atmosphere. The mixture was allowed to stir for 1 hour and was then quenched with saturated aqueous NaHCO₃. The mixture was filtered through a celite pad, and the filtrate was partitioned between DCM and saturated aqueous NaHCO₃. The organic layer was dried over MgSO₄, filtered, and concentrated under reduced pressure. The resulting residue was purified on a silica gel column eluting with hexanes and ethyl acetate to provide compounds 14 and 15 in a 2:1 ratio.

Compound 15 was treated with TBAF (2.5 equivalents) in THE. After starting material was consumed, the reaction mixture was concentrated under reduced pressure and purified by reverse phase to obtain compound 16.

Compound 15 was treated under the same conditions as compound 6 followed by treatment with TBAF to obtain compound 17.

### Example 10

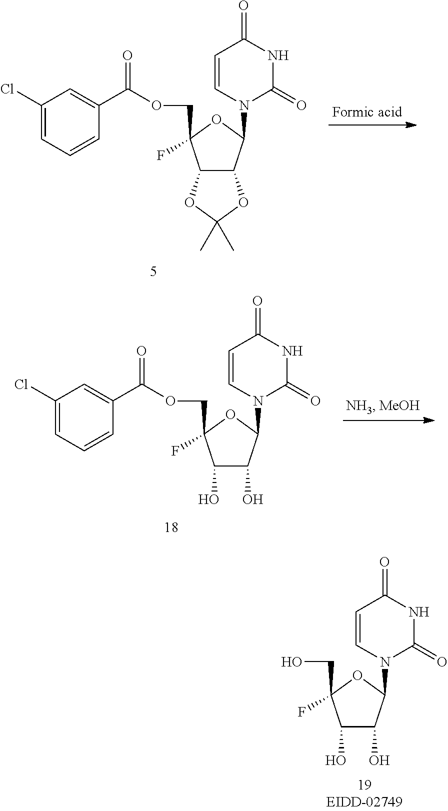 [SMILES: CC1(C)O[C@H]2[C@H](n3ccc(=O)[nH]c3=O)O[C@](F)(COC(=O)c3cccc(Cl)c3)[C@H]2O1 ; O=C(OC[C@@]1(F)O[C@@H](n2ccc(=O)[nH]c2=O)[C@H](O)[C@@H]1O)c1cccc(Cl)c1 ; O=c1ccn([C@@H]2O[C@](F)(CO)[C@@H](O)[C@H]2O)c(=O)[nH]1]

A round bottom flask was charged with compound 5 (0.250 g, 0.567 mmol) and formic acid, 80% aq. (Volume: 10 mL). The mixture was stirred at room temperature under nitrogen overnight. After stirring 20 h, all volatiles were removed by rotary evaporation. The residue was taken up in MeOH and immobilized on Celite. Flash chromatography on the Isco (24 g column, 1 to 15% MeOH in DCM) gave a white powdery solid 90-95% pure by NMR. The white powder was taken up in a 5:1 water:MeCN mixture, and reverse phase flash chromatography on the Isco (100 g C18 column, 100% water to 100% MeCN) gave good separation of the impurity. The fractions containing desired product were concentrated, taken up in 5:1 water:MeCN, frozen in a dry ice bath, and lyophilized to provide compound 18.

A round bottom flask was charged with compound 18 (3.53 g, 8.8 mmol) and ammonia in MeOH (34.3 ml, 240 mmol) at 0° C. The mixture was allowed to stir for 5 hours, at which point all starting material was consumed. The mixture was concentrated by rotary evaporation to give ˜4 g crude as a yellow oil. The crude was taken up in DCM, and flash chromatography on the Isco (120 g column, 1 to 5% MeOH in DCM) gave compound 19. EIDD-02749, (2.20 g, 7.28 mmol, 91% yield) as a white powdery solid.

### Example 11

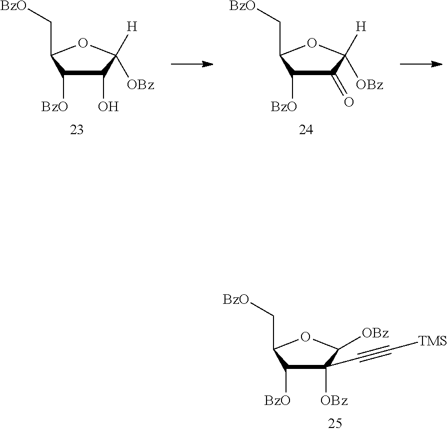

To a stirred solution of DMP (27.5 g, 64.9 mmol) in DCM (162 mL, 0.2M) was cooled to 0° C. and 23 (15 g, 32.4 mmol) was added. The reaction was stirred at 0° C. and allowed to warm to room temperature. After stirring for 18 hours the reaction mixture was concentrated under reduced pressure to a past which was then slurried in 100 mL ethyl ether followed by filtration through a 50 g pad of silica/mag sulfate 1:1 by mass and washed with a total of 400 mL ethyl ether. The ether layer was washed with 2.5 g of sodium thiosulfate in 15 mL water then 2×30 mL cooled sodium bicarbonate, and finally with 30 mL brine. The filtrate was then dried over sodium sulfate, filtered, and concentrated under reduced pressure to provide a foam which was used without further purification. Before use in the next step, a solution of ketone (32.6 mmol) in DCM (200 mL) was prepared and stirred overnight over 5 g of magnesium sulfate at room temperature. After 18 hours of stirring, the solution was filtered and concentrated under reduced pressure.

To a −78° C. solution of TMS Ethylene (11.4 mL, 80 mmol) in dry THF (100 mL) under argon was added butyl lithium (30.5 mL, 2.5M hexanes, 76 mmol). After 30 minutes of stirring, lithiated alkyne was cannulated into a −78° C. suspension of anhydrous CeCl₃ (33.5 g, 90 mmol, dried overnight 150° C. under high vacuum) in dry THF (130 mL) with 2×15 mL rinses of THF. After 90 minutes of stirring, a solution of 24 (32.4 mmol) in dry THF (50 mL) was added via cannula (2×10 mL rinse THF). After 3 hours of stirring, the resulting solution was quenched with saturated aqueous ammonium chloride (100 mL). The reaction was warmed to room temperature and filtered through a celite pad. The celite pad was washed with ethyl ether (3×100 mL) and with saturated aqueous ammonium chloride (100 mL). The filtrate was separated and the organics were washed with saturated aqueous ammonium chloride (100 mL) and brine (100 mL). The filtrate was dried over sodium sulfate, filtered, and concentrated under reduced pressure to provide an oil which was purified by silica gel chromatography 10-50% ethyl acetate in hexanes to provide the product as a mixture of anomers.

To a stirred 0° C. solution of the above product (32.4 mmol) in dry DCM (163 mL, 0.2M) under argon was added sequentially triethyl amine (18 mL, 130 mmol) DMAP (3.98 g, 32.4 mmol), and benzoyl chloride (9.46 mL, 82 mmol). After stirring for 16 hours, the reaction was concentrated under reduced pressure and then slurried in 200 mL ethyl ether and filtered. The organics were concentrated under reduced pressure to provide a paste which was purified by silica gel chromatography eluting with 10-25% ethyl acetate in hexanes to provide 25 as a mixture of anomers. Compound 25 can then be subjected to general base coupling conditions followed by the appropriate deprotection conditions.

### Example 12

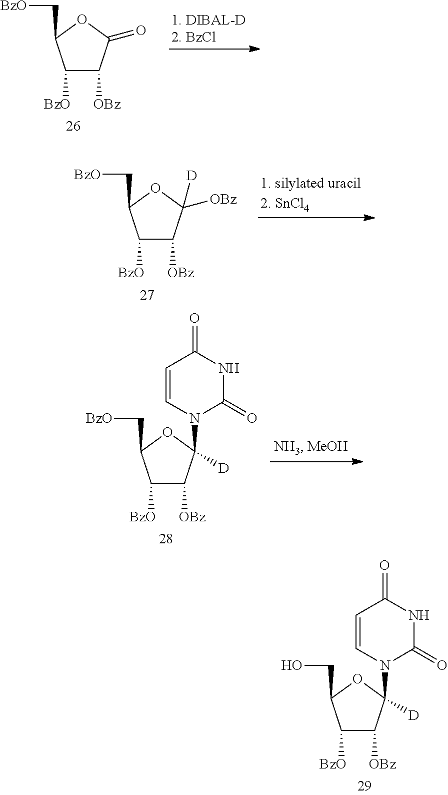

The lactone (0.0325 mol) was added to a dry flask under an argon atmosphere and was then dissolved in dry THE (250 mL). The solution was then cooled to −78° C. and a DIBAL-D solution in toluene (0.065 mol) was added dropwise. The reaction was allowed to stir at −78° C. for 3-4 hours. The reaction was then quenched with the slow addition of water (3 mL). The reaction was then allowed to stir while warming to room temperature.

The mixture was then diluted with two volumes of diethyl ether and was then poured into an equal volume of saturated sodium potassium tartrate solution. The organic layer was separated, dried over MgSO₄, filtered, and concentrated under reduced pressure. The residue was purified on silica eluting with hexanes/ethyl acetate. The resulting lactol, as a solution in dry DCM, was then treated with benzoyl chloride, trimethylamine, and DMAP.

The reaction was allowed to stir at 0° C. until all the starting material was consumed. Next, the reaction mixture was washed with water and then brine. The organic layer was dried over MgSO₄, filtered, and concentrated under reduced pressure. The product was purified on silica eluting with hexanes/ethyl acetate.

To a stirred suspension of uracil (3.92 g, 2 eq) in HMDS (18 mL) was added ammonium sulfate (230mgs, 0.1 eq). The suspension was then refluxed 18h to obtain a clear solution. The solution was cooled to room temperature and concentrated under reduced pressure to a paste. Sugar 27 was dissolved in 1,2-dichloroethane (120 mL) and concentrated under reduced pressure to about 80 mL. The sugar solution was then cannulated into the flask containing silylated base with 2×20 mL rinses of DCE. The reaction was cooled to 0° C. and then tin tetrachloride was added dropwise over 5 minutes. After 30 minutes of stirring, the reaction was allowed to warm to room temperature and was stirred for a further 18 hours overnight. The reaction was charged with 10 g sodium bicarbonate and 10 g celite. 10 mL saturated aqueous sodium bicarbonate was added dropwise (gas evolution occurred). After the quench, the reaction was allowed to stir 30 minutes and then was filtered through a celite pad. The pad was washed with DCM (2×150 mL) and the combined organics were washed with 100 mL saturated aqueous sodium bicarbonate. The organics were collected, dried over sodium sulfate, filtered and concentrated under reduced pressure to provide a brown paste that was purified by silica gel chromatography eluting with 25-100% ethyl acetate in hexanes.

A round bottom flask was charged with compound 28 and ammonia in MeOH at 0° C. The mixture was allowed to stir for 5 hours, at which point all starting material was consumed. The mixture was concentrated by rotary evaporation to give ˜4 g crude as a yellow oil. The crude was taken up in DCM, and flash chromatography on the Isco (120 g column, 1 to 5% MeOH in DCM) gave compound 29.

### Example 13

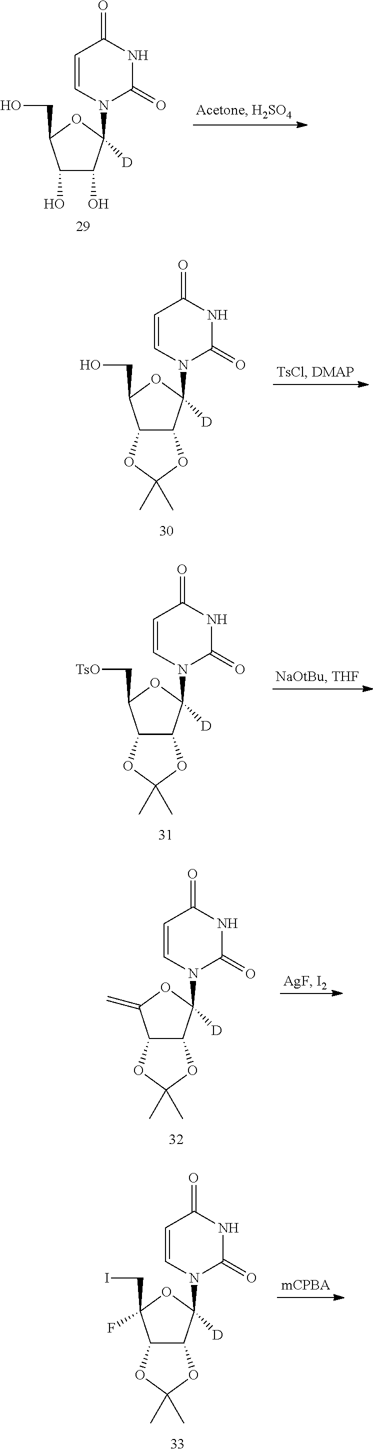 [SMILES: [2H][C@@]1(n2ccc(=O)[nH]c2=O)O[C@H](CO)[C@@H](O)[C@H]1O ; [2H][C@@]1(n2ccc(=O)[nH]c2=O)O[C@H](CO)[C@H]2OC(C)(C)O[C@H]21 ; [2H][C@@]1(n2ccc(=O)[nH]c2=O)O[C@H](COS(=O)(=O)c2ccc(C)cc2)[C@H]2OC(C)(C)O[C@H]21 ; [2H][C@@]1(n2ccc(=O)[nH]c2=O)OC(=C)[C@H]2OC(C)(C)O[C@H]21 ; [2H][C@@]1(n2ccc(=O)[nH]c2=O)O[C@](F)(CI)[C@H]2OC(C)(C)O[C@H]21]

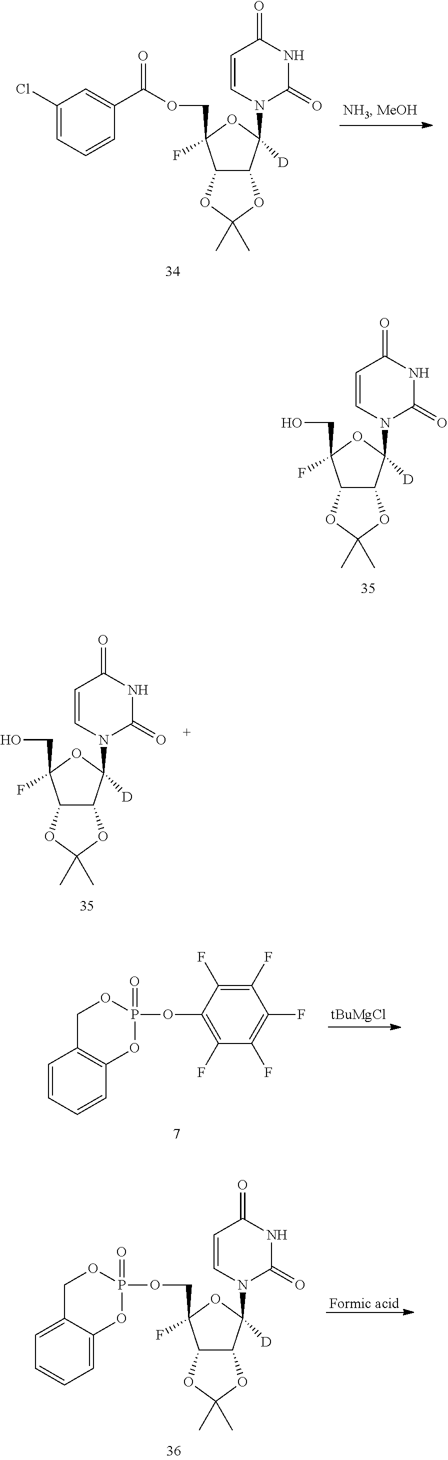

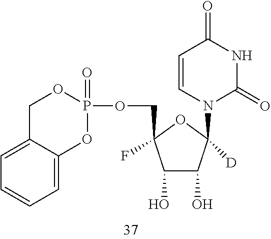 [SMILES: [2H][C@@]1(n2ccc(=O)[nH]c2=O)O[C@](F)(COP2(=O)OCc3ccccc3O2)[C@@H](O)[C@H]1O]

A 1 L rbf was charged with compound 29 (36.6 g, 150 mmol) and acetone (Volume: 700 ml) with stirring under nitrogen at rt. The slurry was treated with concentrated sulfuric acid (0.800 ml, 15.00 mmol) and the mixture was stirred at rt overnight. After stirring 16 h, triethylamine (41.8 ml, 300 mmol) was added all at once, the mixture was stirred 30 min, and then concentrated by rotary evaporation to give a sticky white solid. The solid was dissolved in boiling iPrOH (˜1.4 L) and allowed to cool overnight at rt. After cooling overnight, small crystals had formed. The flask was placed in the freezer for 3 h and more crystals formed. The mixture was vacuum filtered, and the solids were washed with ice-cold iPrOH (2×200 mL) and ice-cold ether (2×200 mL). The solid was recovered to give compound 30 (21.75 g, 77 mmol, 51.0% yield) as a white powdery solid.

A round bottom flask was charged with compound 30 (21.75 g, 77 mmol) and DCM (219 ml) and the mixture was stirred under nitrogen. Solid 4-DMAP (23.37 g, 191 mmol) was added all at once, and the mixture was stirred at rt until all solids dissolved. The mixture was cooled to 0° C., and tosyl chloride (17.50 g, 92 mmol) was added portionwise as a solid over 5 min. The mixture was stirred at rt for 1 h until all starting material was consumed. The mixture was transferred to a separatory funnel, and the organic layer was washed with 1 N HCl (2×200 mL), sat. aq. NaHCO₃ (1×200 mL), and brine (1×200 mL), then dried over Na₂SO₄, filtered and concentrated by rotary evaporation to give compound 31 (34.52 g, 74.8 mmol, 98% yield) as a white solid.

To a stirred solution of compound 31 (3.95 g, 9.01 mmol) in THE (30 mL) at 0° C. under nitrogen. Solid potassium tert-butoxide (3.03 g, 27.0 mmol) was added all at once, the reaction mixture turned into a yellow slurry. The mixture was stirred at 0° C. for 2 h. Silica gel (6 g) and Celite (14 g) were added along with more THF, and the mixture was concentrated by rotary evaporation. Flash chromatography on the Isco (80 g column, 1 to 5% MeOH in DCM) gave compound 32 (2.17 g, 8.15 mmol, 90% yield) as a white powdery solid.

A round bottom flask was charged with a stir bar, compound 32 (2.17 g, 8.15 mmol), silver(I) fluoride (5.17 g, 40.8 mmol), and DCM (Volume: 152 ml, Ratio: 14) at 0° C. To this vigorously stirred mixture was added a solution of iodine (4.14 g, 16.30 mmol) in THE (Volume: 10.87 ml, Ratio: 1.000) dropwise via syringe over 40 min. After addition was complete, the mixture was stirred another 15 min at 0° C., then a 1:1 mixture of sat. aq.

NaHCO₃:sat. aq. Na₂S₂O₃ was added (75 mL) and the whole mixture was filtered through a Celite pad, washing with DCM (2×50 mL). The filtrates were transferred to a separation funnel, and the organic layer was dried over Na₂SO₄, filtered, and concentrated by rotary evaporation to give 4 g. Flash chromatography on the Isco (120 g column, 5 to 25% EtOAc in DCM) gave compound 33 (2.06 g, 5.00 mmol, 61.3% yield) as a pale yellow flaky solid.

A round bottom flask was charged with compound 33 (10.76 g, 26.1 mmol), tetrabutylammonium sulfate (8.86 g, 26.1 mmol), potassium hydrogen phosphate dibasic trihydrate (8.94 g, 39.2 mmol), DCM (Volume: 1088 ml, Ratio: 5) and water (Volume: 218 ml, Ratio: 1.000) and the biphasic mixture was stirred vigorously at rt. To this mixture was added solid mCPBA, 77% w/w (29.3 g, 131 mmol) all at once and the mixture was stirred at rt overnight. After stirring 20 h at rt, all SM had been consumed by TLC analysis. The mixture was quenched by slow addition of sat. aq. Na₂S₂O₃ (375 mL) followed by sat. aq.

Na₂CO₃ (375 mL). The organic layer was removed, and the aqueous layer was extracted with DCM (1×450 mL). The combined organic layers were dried over Na₂SO₄, filtered, and concentrated by rotary evaporation to give 22 g crude. The crude was taken up in DCM, and flash chromatography on the Isco (330 g column, 5 to 25% EtOAc in DCM) gave 10 g of semipure product. The compound was taken up in DCM, and flash chromatography on the Isco (330 g column, 5 to 70% EtOAc in hexanes) gave compound 34 (6.91 g, 15.68 mmol, 60.0% yield) as an off-white flaky solid.

A round bottom flask was charged with compound 34 (3.53 g, 8.0 mmol) and ammonia in MeOH (34.3 ml, 240 mmol) at 0° C. The mixture was stirred for 5 h, at which point all starting material was consumed. The mixture was concentrated by rotary evaporation to give ˜4 g crude as a yellow oil. The crude was taken up in DCM, and flash chromatography on the Isco (120 g column, 1 to 5% MeOH in DCM) gave compound 35 (2.20 g, 7.28 mmol, 91% yield) as a white powdery solid.

A 1 L 3-neck RBF equipped with temperature probe, overhead stirrer and addition funnel (argon inlet) was charged with phosphorus oxychloride (15.50 ml, 166 mmol) in THE (300 ml), evacuated and purged with argon 3×, then cooled to <−70° C. using dry ice/acetone. A solution of 2-(hydroxymethyl)phenol (18.77 g, 151 mmol) and triethylamine (44.3 ml, 317 mmol) in 200 mL of THE was slowly added via addition funnel over 30 minutes. The resulting light tan mixture was slowly warmed to RT and stirred for 3 hrs.

Cooled to 0° C. using an ice bath and added triethylamine (25.3 ml, 181 mmol), then slowly added a THE (100 mL) solution of 2,3,4,5,6-pentafluorophenol (25.05 g, 136 mmol) to the rapidly stirred mixture. Warmed to RT and monitored by TLC (25% EtOAc/hexanes). SM consumed in <2 hrs, only product (Rf=0.5) present. The oil was purified by SGC (glass column, 10-25% EtOAc/hexanes), fractions containing product were pooled and concentrated under reduced pressure to yield compound 7 (41.2 g, 117 mmol, 77% yield) as a white solid.

To a stirred solution of compound 35 (1.95 g, 6.45 mmol) in THE (Volume: 96 ml, Ratio: 5) at 0° C. under nitrogen, was added a solution of tert-butylmagnesium chloride, 1.0 M in THE (14.19 ml, 14.19 mmol) dropwise via syringe. A white precipitate formed; the mixture was warmed to rt and stirred for 30 min, then recooled to 0° C. A solution of compound 7 (5.68 g, 16.13 mmol) in THE (Volume: 19.20 ml, Ratio: 1.000) was added dropwise via syringe, and the mixture was warmed to rt and stirred overnight. After 18 h stirring, a little SM remained and one slightly less polar product had formed. The mixture was quenched by addition of solid NH₄Cl (2 g) and the mixture was immobilized on Celite.

Flash chromatography on the Isco (220 g column, 1 to 5% MeOH in DCM) gave 1.94 g of a white solid that consisted of desired product and pentafluorophenol. The solid was taken up in DCM and washed with sat. aq. NaHCO₃ (3×100 mL). The organic layer was dried over Na₂SO₄, filtered, and concentrated by rotary evaporation to give compound 36 (1.70 g, 3.61 mmol, 56.0% yield) as a white powdery solid.

A round bottom flask was charged with compound 36 (0.250 g, 0.532 mmol) and formic acid, 80% aq. (Volume: 10 mL). The mixture was stirred at rt under nitrogen overnight. After stirring 20 h, all volatiles were removed by rotary evaporation. The residue was taken up in MeOH and immobilized on Celite. Flash chromatography on the Isco (24 g column, 1 to 15% MeOH in DCM) gave a white powdery solid, 175 mg, 90-95% pure by NMR. The white powder was taken up in a 5:1 water:MeCN mixture, and reverse phase flash chromatography on the Isco (100 g C18 column, 100% water to 100% MeCN) gave good separation of the impurity. The fractions containing desired product were concentrated, taken up in 5:1 water:MeCN, frozen in a dry ice bath, and lyophilized to provide compound 37.

### Example 14

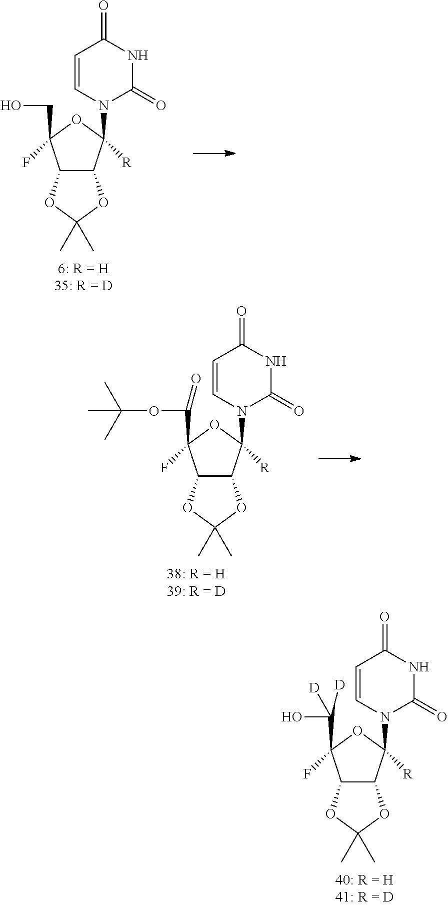

Either the nucleoside 6 or 35 was suspended in methylene chloride (40 mL, partially soluble). After stirring at rt for 30 min the mixture was treated sequentially with PDC, acetic anhydride and then tert-butanol. The mixture was allowed to stir at room temperature. TLC (5% methanol in DCM) and LCMS indicated only a small amount of remaining starting material at 4 hours. The mixture was filtered through a pad of silica gel that was loaded into a 150 mL fritted funnel. The silica was eluted with ethyl acetate. The collected filtrate was concentrated by under reduced pressure. The crude dark oil was purified by chromatography over silica gel (25 mm×175 mm) with 2:1 hexanes:ethyl acetate to ethyl acetate gradient. The pure fractions were collected and concentrated under reduced pressure to give of a white gum. The material was placed under high vacuum for 2 days to provide either compound 38 or 39. The material was used in the next step without further purification.

The 5′-protected nucleoside 38 or 39 was dissolved in 200 proof ethanol and was then treated with solid sodium borodeuteride. The mixture became homogeneous and was then heated to 80° C. After 12h, a white/pale yellow precipitate formed. The mixture was allowed to cool to rt. TLC (5% methanol in methylene chloride) indicates complete conversion of starting material. The mixture was cooled to 0° C. with an ice-bath and then slowly quenched with acetic acid (approximately 1 mL). The clear solution was warmed to rt and then partitioned between ethyl acetate (30 mL) and brine (3 mL). The organic phase was concentrated and then purified by chromatography over silica gel (19 mm×180 mm) using a mobile phase of 5% methanol in methylene chloride to provide compound 40 or 41.

Compounds 40 and 41 can then be deprotected to obtain the unprotected ribonucleoside using 80% formic acid as described previously. Additionally, Compounds 40 and 41 can be conjugated to prodrug reagent 7 followed by deprotection as described previously.

### Example 15

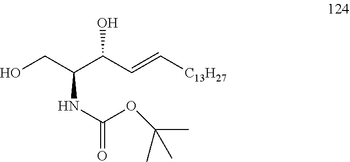

Prepared according to Boumendjel, Ahcene and Miller, Stephen Journal of Lipid Research 1994, 35, 2305.

A mixture of sphingosine (450 mg, 1.50 mmol) and di-tert-butyl dicarbonate (0.656 g, 3.01 mmol) in methylene chloride (100 mL) at 4° C. was treated dropwise with diisopropylethylamine (0.53 mL, 3.01 mmol). After gradual warming to rt, the mixture was stirred for an additional 12 h and then diluted with methylene chloride (100 mL) followed by a wash with water (30 mL) and brine (30 mL). The organic phase was dried over sodium sulfate, filtered and concentrated to dryness. The crude residue was purified by flash column chromatography over silica gel (19 mm×175 mm) using 50% ethyl acetate in hexanes to give N-tert-butyloxycarbonyl-sphingosine (540 mg, 90%) as a white solid.

¹H NMR (300 MHz, Chloroform-d) δ 5.77 (dt, J=15.4, 8.4 Hz, 1H), 5.52 (dd, J=15.4, 8.4 Hz, 1H), 3.93 (dd, J=11.4, 3.7 Hz, 1H), 3.70 (dd, J=11.4, 3.7 Hz, 1H), 3.59 (s, 3H), 2.05 (q, J=7.0 Hz, 2H), 1.52 (s, 9H), 1.25 (s, 22H), 0.87 (t, J=6.5 Hz, 3H).

### Example 16

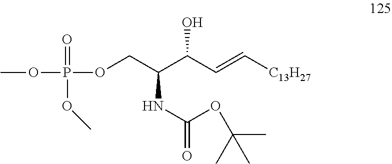

N-tert-Butyloxycarbonyl-sphingosine 124(540 mg, 1.35 mmol) was rendered anhydrous by co-evaporation with anhydrous pyridine (2×12 mL). The residue was then dissolved in anhydrous pyridine and treated with carbon tetrabromide (622 mg, 1.88 mmol).

The mixture was cooled to 0° C. and treated dropwise with a solution of trimethylphosphite (0.25 mL, 2.10 mmol) in anhydrous pyridine (3 mL) over a 30 min period. After an additional 12 h at rt, both LCMS and tlc (5% methanol in methylene chloride) analysis indicated complete conversion. The mixture was quenched with water (2 mL) and then concentrated to dryness. The resulting dark oil was dissolved in ethyl acetate (150 mL) and washed with 3% HCL solution (2×20 mL) followed by saturated sodium bicarbonate solution (30 mL). The organic layer was dried over sodium sulfate, filtered and concentrated. The crude residue was purified by flash column chromatography over silica gel (19 mm×175 mm) using 2% methanol in methylene chloride to give N-tert-butyloxycarbonyl-sphingosine-1-O-dimethylphosphate 125 (350 mg, 51%) as a gum.

¹H NMR (400 MHz, Chloroform-d) δ 5.82 (dt, J=15.4, 7.1 Hz, 1H), 5.48 (dd, J=15.4, 7.1 Hz, 1H), 4.99 (d, J=8.9 Hz, 1H), 4.32 (ddd, J=10.7, 8.0, 4.6 Hz, 1H), 4.11 (ddt, J=10.7, 7.4, 3.1 Hz, 2H), 3.77 (dd, J=11.1, 2.1 Hz, 6H), 2.01 (q, J=7.1 Hz, 2H), 1.41 (s, 9H), 1.34 (m, 2H), 1.23 (m, 20H), 0.86 (t, J=6.4 Hz, 3H).

³¹P NMR (162 MHz, Chloroform-d) δ 2.00.

MS C17H25NO4 [M+Na+]; calculated: 330.2, found: 330.2.

### Example 17

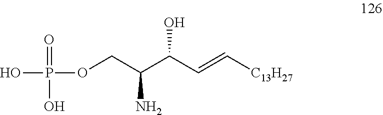

A solution of N-tert-butyloxycarbonyl-sphingosine-1-O-dimethylphosphate 125 (350 mg, 0.689 mmol) in anhydrous methylene chloride (8 mL) was treated dropwise with trimethylsilyl bromide (0.45 mL, 3.45 mmol) at 0° C. After warming to room temperature, the mixture was allowed to stir at rt for 6 h and then concentrated to dryness. The resulting residue was co-evaporated with methylene chloride to remove excess trimethylsilyl bromide and then treated with 66% aqueous THE (6 mL). The resulting precipitate was collected by filtration to give sphingosine-1-phosphate 126 (218 mg, 83%) as a white solid.

¹H NMR (400 MHz, Methanol-d₄+CD₃CO₂D) δ 5.84 (dt, J=15.5, 6.7 Hz, 1H), 5.46 (dd, J=15.5, 6.7 Hz, 1H), 4.33 (t, J=6.0 Hz, 1H), 4.13 (ddd, J=11.8, 7.7, 3.6 Hz, 1H), 4.03 (dt, J=11.8, 8.4 Hz, 1H), 3.47 (ddd, J=8.3, 4.8, 3.2 Hz, 1H), 2.10-1.99 (m, 2H), 1.37 (m, 2H), 1.24 (m, 20H), 0.83 (t, J=6.4 Hz, 3H).

³¹P NMR (162 MHz, Chloroform-d) δ 0.69.

MS C₁₈H₃₈NO₅P [M−H⁺]; calculated: 378.2, found: 378.2.

### Example 18

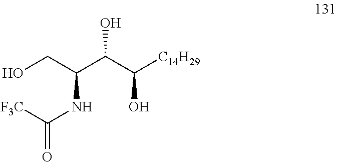

To a slurry of phytosphingosine (4 g, 12.6 mmol) and anhydrous powdered potassium carbonate (5.22 g, 37.8 mmol) in methylene chloride (85 mL) was added trifluoroacetic anhydride (1.96 mL, 13.9 mmol). The mixture was stirred at rt for 18 h and then diluted with methylene chloride (500 mL). The mixture was washed with water (100 mL). Methanol (60 mL) was added to break the emulsion. The organic phase was then dried over sodium sulfate, filtered and concentrated to give 131 (4.9 g, 94%) as a white solid

¹H NMR (400 MHz, DMSO-d₆) δ 8.90 (s, 1H), 4.90-4.68 (m, 1H), 4.56 (d, J=6.1 Hz, 1H), 4.43 (s, 1H), 3.97 (d, J=7.6 Hz, 1H), 3.65 (d, J=10.8 Hz, 1H), 3.46 (t, J=10.2 Hz, 1H), 3.32-3.16 (m, 1H), 1.42 (tt, J=15.7, 7.5 Hz, 2H), 1.20 (s, 24H), 0.83 t, J=6.8 Hz, 3H).

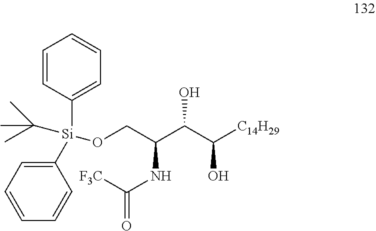

### Example 19

N-Trifluoroacetyl-phytosphingosine (131, 1.88 g, 4.5 mmol) in anhydrous pyridine (23 mL) was treated with DMAP (56 mg, 0.45 mmol) and then dropwise with tert-butyldiphenylsilyl chloride (1.38 g, 5.0 mmol). After 18 h concentrated to dryness. The resulting residue was dissolved in ethyl acetate (200 mL) and washed with saturated ammonium chloride (2×50 mL) and then brine (50 mL). The aqueous phases was back-extracted with ethyl acetate (50 mL). Combined organic phases were dried over sodium sulfate and concentrated to give crude 1-O-tert-Butyldiphenylsilyl-2-N-trifluoroacetyl-phytosphingosine 132 (3 g, 100%) as a gum. The material was used in the next step without further purification.

¹H NMR (400 MHz, Chloroform-d) δ 7.62 (m, 2H), 7.60-7.56 (m, 2H), 7.47-7.31 (m, 6H), 7.07 (d, J=8.4 Hz, 1H), 4.23 (dd, J=8.5, 4.1 Hz, 1H, 4.04 (dt, J=11.0, 2.5 Hz, 1H), 3.82 (ddd, J=11.0, 4.3, 1.8 Hz, 1H), 3.64 (dq, J=10.6, 6.0, 4.3 Hz, 2H), 1.45 (m, 2H), 1.39-1.15 (m, 24H), 1.05 (m, 9H), 0.94-0.80 (t, J=6.9 Hz 3H).

### Example 20

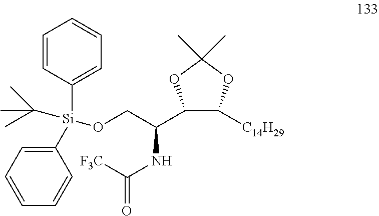

A solution of 1-O-tert-Butyldiphenylsilyl-2-N-trifluoroacetyl-phytosphingosine 132 (3 g, 4.5 mmol) in 1/1 (v/v) 2,2-dimethoxypropane/THF was treated with catalytic amount of p-toluenesulfonic acid (87 mg, 0.45 mmol) and allowed to stir for 16 h at rt. The mixture was quenched with saturated sodium bicarbonate (30 mL) and then excess THF/2,2-dimethoxypropane was removed under vacuum. The mixture was extracted with ethyl acetate (200 mL). After washing with brine, the organic layer was dried over sodium sulfate, filtered and concentrated. The crude oil was purified by column chromatography (25 mm×175 mm) over silica gel with a hexanes/ethyl acetate mobile phase to give 133(2.45 g, 78%).

¹H NMR (400 MHz, Chloroform-d) δ 7.68-7.63 (m, 2H), 7.63-7.57 (m, 2H), 7.39 (m, 6H), 6.54 (d, J=9.4 Hz, 1H), 4.23 (dd, J=8.2, 5.6 Hz, 1H), 4.12 (ddd, J=13.3, 6.9, 3.8 Hz, 2H), 3.96 (dd, J=10.5, 3.9 Hz, 1H), 3.69 (dd, J=10.5, 2.9 Hz, 1H), 1.52-1.36 (m, 2H), 1.33 (s, 3H), 1.31 (s, 3H), 1.24 (m, 24H), 1.03 (s, 9H), 0.86 (t, J=53.7, 6.9 Hz, 3H).

### Example 21

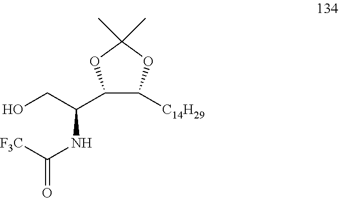

A solution of 1-O-tert-Butyldiphenylsilyl-3,4-O-isopropylidene-2-N-trifluoroacetyl-phytosphingosine 133 (2.45 g, 3.54 mmol) in THE (18 mL) was treated with tetrabutylammonium fluoride (4.25 mL of a 1.0 M solution in THF, 4.25 mmol) and stirred at rt for 12 h. The mixture was diluted with ethyl acetate (100 mL) and saturated ammonium chloride (2×50 mL) and then brine (50 mL). The organic phase was dried over sodium sulfate, filtered, and concentrated to give a white solid that was further purified by column chromatography (25 mm×175 mm) over silica gel with a 9:1 hexanes: ethyl acetate mobile phase to afford 134(1.5 g, 93%) as a white solid.

¹H NMR (300 MHz, Chloroform-d) δ 6.92 (d, J=8.7 Hz, 1H), 4.31-4.16 (m, 2H), 4.11 (dq, J=11.7, 3.7 Hz, 1H), 4.00 (dd, J=11.5, 2.6 Hz, 1H), 3.70 (dd, J=11.5, 3.6 Hz, 1H), 1.48 (s, 3H), 1.35 (s, 3H), 1.25 (m, 26H), 0.88 (t, J=6.9 Hz 3H).

### Example 22

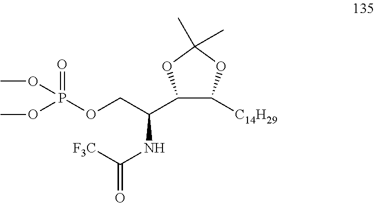

A solution of 3,4-O-Isopropylidene-2-N-Trifluoroacetyl-phytosphingosine 134(630 mg, 1.39 mmol) was rendered anhydrous by co-evaporation with anhydrous pyridine (2×12 mL). The residue was then dissolved in anhydrous pyridine (12 mL) and treated with carbon tetrabromide (533 mg, 1.67 mmol). The mixture was cooled to 0° C. and treated dropwise with a solution of trimethylphosphite (0.23 mL, 1.95 mmol) in anhydrous pyridine (3 mL) over a 30 min period. After an additional 12 h at rt, both LCMS and tlc (5% methanol in methylene chloride) analysis indicated complete conversion. The mixture was quenched with water (2 mL) and then concentrated to dryness. The resulting dark oil was dissolved in ethyl acetate (100 mL) and washed with 3% HCL solution (2×20 mL) followed by saturated sodium bicarbonate solution (30 mL). The organic layer was dried over sodium sulfate, filtered and concentrated. The crude residue was purified by flash column chromatography over silica gel (19 mm×175 mm) using 2% methanol in methylene chloride to give 135 (650 mg, 83%).

¹H NMR (300 MHz, Chloroform-d) δ 7.42 (d, J=8.8 Hz, 1H), 4.36 (td, J=10.9, 5.0 Hz, 1H), 4.25 (m, 1H), 4.19 (m, J=6.5, 2.0 Hz, 3H), 3.77 (dd, J=11.2, 7.5 Hz, 6H), 1.44 (s, 3H), 1.33 (s, 3H), 1.25 (m, 26H), 0.87 (t, J=6.6 Hz, 3H).

³¹P NMR (121 MHz, Chloroform-d) δ 1.69.

MS C₂₅H₄₇F₃NO₇P [M−H⁺]; calculated: 560.3, found: 560.2.

### Example 23

### 3,4-O-Isopropylidene-2-N-trifluoroacetyl-phytosphingosine-1-phosphate (136)

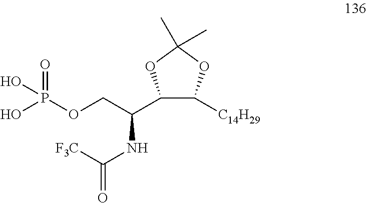

A solution of 3,4-O-Isopropylidene-2-N-trifluoroacetyl-phytosphingosine-1-O-dimethylphosphate 135 (650 mg, 1.16 mmol) in anhydrous methylene chloride (12 mL) was treated dropwise with trimethylsilyl bromide (0.81 mL, 6.23 mmol) at 0° C. After 12 h at rt, the mixture was concentrated to dryness and the resulting residue co-evaporated with methylene chloride (3×50 mL) to remove excess trimethylsilyl bromide. The residue then was dissolved in cold (4° C.) solution of 1% NH₄OH while maintaining pH 7-8. After 10 min at rt, the mixture was concentrated to dryness, and the resulting solid triturated with methanol/acetonitrile. The solid was collected by filtration, washed with acetonitrile, and dried under high vacuum to give 136 (500 mg, 75%) as a white solid.

¹H NMR (300 MHz, Methanol-d₄) δ 4.31 (dd, J=8.7, 5.4 Hz, 1H), 4.09 (m, 4H), 1.42 (s, 3H), 1.36 (s, 3H), 1.31 (m, 26H), 0.89 (t, J=6.4 Hz, 3H).

³¹P NMR (121 MHz, Methanol-d₄) δ 1.28.

¹⁹F NMR (282 MHz, Methanol-d₄) a ˜77.13.

HRMS C₂₃H₄₂F₃NO₇P [M−H⁺]; calculated: 532.26565, found: 532.26630.

### Example 24

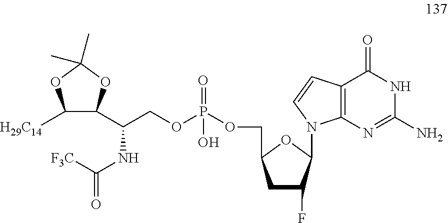

A mixture of N-trifluoroacetyl-phytosphingosine-1-phosphate 136(200 mg, 0.373 mmol) and 2′,3′-dideoxy-2′-fluoro-7-deazaguanine (100 mg, 0.373 mmol) was rendered anhydrous by co-evaporation with anhydrous pyridine (3×10 mL). The resulting residue then was dissolved in anhydrous pyridine (4 mL) and treated with diisopropylcarbodiimide (127 mg, 1.01 mmol) and HOBt (60 mg, 0.447 mmol). After 24 h at 75° C., the reaction mixture was cooled to rt and concentrated to dryness. The crude material was purified by flash column chromatography (19 mm×170 mm) over silica gel using a solvent gradient from 5 to 7.5% methanol in chloroform with 1% (v/v) NH₄OH to give 137(80 mg, 27%) as a white solid.

¹H NMR (300 MHz, Methanol-d4) δ 6.88 (d, J=3.8 Hz, 1H), 6.46 (d, J=3.8 Hz, 1H), 6.24 (d, J=19.9 Hz, 1H), 5.34 (dd, J=52.4, 4.6 Hz, 1H), 4.53 (s, 1H), 4.34-3.97 (m, 6H), 2.63-2.17 (m, 2H), 1.40 (s, 3H), 1.30 (s, 3H), 1.27 (m, 26H), 0.89 (t, J=6.6 Hz, 3H).

³¹P NMR (121 MHz, Methanol-d4) δ 12.50.

¹⁹F NMR (282 MHz, Methanol-d₄) δ −77.10, −179.69-−180.25 (m).

MS C₃₄H₅₂2F₄N₅O₉P [M−H⁺]; calculated: 781.3, found: 782.2.

### Example 25

### Experimental Procedure for Synthesis of Prodrugs

A solution of isopropyl 2-((chloro(phenoxy)phosphoryl)amino)propanoate (0.397 g, 1.300 mmol) in anhydrous THE (5 ml) was added to a −78° C. stirred solution of 2′-deoxy-2′-fluoronucleoside (0.812 mmol) and 1-methyl-1H-imidazole (0.367 ml, 4.63 mmol) in pyridine (10.00 ml). After 15 min the reaction was allowed to warm to room temperature and was stirred for an additional 3 hours. Next, the solvent was removed under reduced pressure. The crude product was dissolved in 120 ml of DCM and was washed with 20 ml 1 N HCl solution followed by 10 ml water. The organic phase was dried over sodium sulfate, filtered and concentrated in vacuo. The residues were separated over silica column (neutralized by TEA) using 5% MeOH in DCM as a mobile phase to yield the respective products as diastereomers.

### Example 26

### N-tert-Butyloxycarbonyl-phytosphingosine (174)

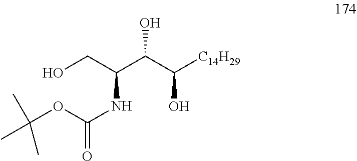

A suspension of phytosphingosine (10.6 g, 33.5 mmol) and triethylamine (5.6 ml, 40.2 mmol) in THE (250 mL) was treated dropwise with di-tert-butyl dicarbonate (8.6 mL, 36.9 mmol). After 12 h at rt, the mixture was concentrated to dryness and the resulting white solid was recrystallized from ethyl acetate (80 mL) and then dried under high vacuum at 35° C. for 12h to give 174(10.5 g, 75%).

¹H NMR (400 MHz, Chloroform-d) δ 5.31 (d, J=8.5 Hz, 1H), 3.89 (d, J=11.1 Hz, 1H), 3.83 (s, 2H), 3.74 (dd, J=11.1, 5.2 Hz, 1H), 3.65 (d, J=8.3 Hz, 1H), 3.61 (d, J=3.9 Hz, 1H), 1.43 (s, 9H), 1.23 (s, 27H), 0.86 (t, J=6.4 Hz, 3H).

### Example 27

### 2-O-tert-Butyldiphenylsilyl-1-N-tert-butyloxycarbonyl-phytosphingosine (175)

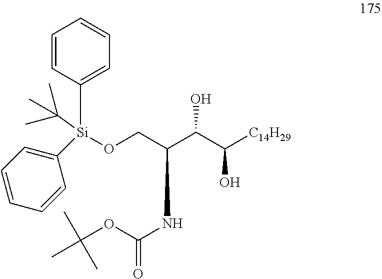

A solution of N-tert-Butyloxycarbonyl-phytosphingosine 174 (9.5 g, 22.65 mmol) and triethylamine (3.8 mL, 27.2 mmol) in anhydrous methylene chloride/DMF (120 mL/10 mL) was treated dropwise with tert-butylchlorodiphenylsilane (7 mL, 27.25 mmol). After 18 h at rt, the mixture was diluted with methylene chloride (200 mL) and washed with 0.2N HCl (100 mL) and then brine (100 mL). The organic phase was dried over sodium sulfate, filtered and then concentrated to give 175 (14.9 g) as an oil which was used in the next reaction without further purification.

¹H NMR (400 MHz, Chloroform-d) δ 5.31 (d, J=8.5 Hz, 1H), 3.89 (d, J=11.1 Hz, 1H), 3.83 (m, 1H), 3.74 (dd, J=11.1, 5.2 Hz, 1H), 3.65 (d, J=8.3 Hz, 1H), 3.61 (d, J=3.9 Hz, 1H), 1.43 (s, 9H), 1.23 (s, 27H), 0.86 (t, J=6.4 Hz, 3H).

### Example 28

### 2-O-tert-Butyldiphenylsilyl-1-N-tert-butyloxycarbonyl-3,4-O-isopropylidene-phytosphingosine (176)

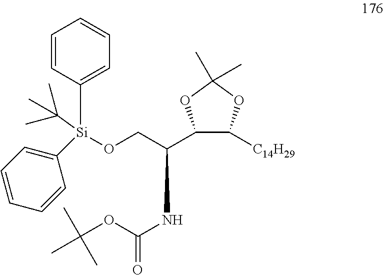

A solution of 2-O-tert-Butyldiphenylsilyl-1-N-tert-butyloxycarbonyl-phytosphingosine (175, 14.9 g, 22.65 mmol) in 1/1 (v/v) THF/2,2-dimethoxypropane was treated with catalytic para-toluenesulfonic acid (860 mg, 4.53 mmol). After 24 h, the mixture was quenched with saturated sodium bicarbonate solution (50 mL). The mixture was concentrated and then dissolved in ethyl acetate (200 mL) and washed with brine (2×50 mL). The organic phase was dried over sodium sulfate, filtered and concentrated to give 176 (15.7 g) as a gum which was used in the next step without further purification.

¹H NMR (400 MHz, Chloroform-d) δ 7.66 (m, 4H), 7.51-7.27 (m, 6H), 4.78 (d, J=10.0 Hz, 1H), 4.18 (dd, J=9.3, 5.5 Hz, 1H), 3.89 (dd, J=9.9, 3.3 Hz, 1H), 3.80 (d, J=9.9 Hz, 1H), 3.72 (d, J=9.9 Hz, 1H), 1.45 (s, 9H), 1.42 (s, 3H), 1.35 (s, 3H), 1.25 (s, 27H), 1.05 (s, 9H), 0.87 (t, J=6.5 Hz, 3H).

### Example 29

### 1-N-tert-butyloxycarbonyl-3,4-O-isopropylidene-phytosphingosine (177)

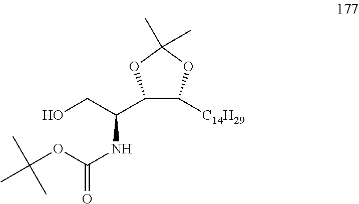

A solution of 2-O-tert-Butyldiphenylsilyl-1-N-tert-butyloxycarbonyl-3,4-O-isopropylidene-phytosphingosine 176 (15.7 g, 22.6 mmol) in THE at 0° C. was treated dropwise with a solution of tetrabutylammonium fluoride (1.0 M in THF, 24.9 mL, 24.9 mmol) over a 20 min period. After 16 h at rt, tlc (3:1 hexanes:ethyl acetate) indicated complete conversion. The mixture was concentrated to dryness and the resulting residue was dissolved in ethyl acetate (300 mL) and washed with water (3×100 mL). The organic phase was dried over sodium sulfate, filtered and concentrated. The resulting oil purified by flash column chromatography (35 mm×180 mm) using a solvent gradient from 25 to 50% ethyl acetate in hexanes to give 177 (7.3 g, 71% over 3 steps) as a white solid.

¹H NMR (400 MHz, Chloroform-d) δ 4.93 (d, J=9.1, 1H), 4.16 (q, J=7.1, 6.4 Hz, 1H), 4.07 (t, J=6.5 Hz, 1H), 3.83 (dd, J=11.1, 2.4 Hz, 1H), 3.76 (m, 1H), 3.67 (dd, J=11.2, 3.6 Hz, 1H), 1.43 (s, 3H), 1.42 (s, 9H), 1.32 (s, 3H), 1.23 (s, 27H), 0.86 (t, J=6.9 Hz, 3H).

### Example 30

### General Procedure for the Preparation of 5′-Phosphoramidate Prodrugs

### Synthesis of Chlorophosphoramidate:

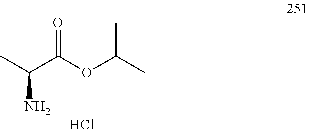

Thionyl chloride (80 g, 49.2 mL, 673 mmol) was added dropwise over a 30 min period to a suspension of L-alanine (50 g, 561 mmol) in isopropanol (500 mL). The mixture was heated to a gentle reflux for 5 h and then concentrated by rotary evaporator (bath set at 60° C.). The resulting thick gum solidified upon trituration with ether (150 ml). The white powder was triturated a second time with ether (150 mL), collected by filtration while under a stream of argon, and then dried under high vacuum for 18h to give (S)-isopropyl 2-aminopropanoate hydrochloride (88 g, 94%).

¹H NMR (400 MHz, DMSO-d₆) δ 8.62 (s, 3H), 5.10-4.80 (m, 1H), 3.95 (q, J=7.2 Hz, 1H), 1.38 (d, J=7.2 Hz, 3H), 1.22 (d, J=4.6 Hz, 3H), 1.20 (d, J=4.6 Hz, 3H).

### Example 31

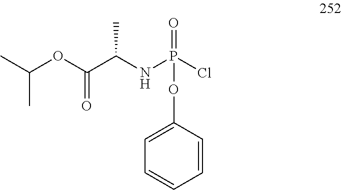 [SMILES: CC(C)OC(=O)[C@H](C)NP(=O)(Cl)Oc1ccccc1]

A solution of phenyl dichlorophosphate (30.9 g, 146 mmol) in dichloromethane (450 mL) was cooled to 0° C. then treated with (S)-isopropyl 2-aminopropanoate hydrochloride (24.5 g, 146 mmol). The mixture was further cooled to −78° C. and then treated dropwise with triethylamine (29.6 g, 40.8 mL, 293 mmol) over a 30 min period. The mixture continued to stir at −78° C. for an additional 2 h and then allowed to gradually warm to rt.

After 18 h the mixture was concentrated to dryness and the resulting gum dissolved in anhydrous ether (150 mL). The slurry was filtered while under a stream of argon, and the collected solid washed with small portions of anhydrous ether (3×30 mL). Combined filtrates were concentrated to dryness by rotary evaporator to give a 1:1 diastereomeric mixture of phosphochloridate (41.5 g, 93%) as pale yellow oil.

¹H NMR (300 MHz, Chloroform-d) δ 7.43-7.14 (m, 5H), 5.06 (m, 1H), 4.55 (dd, J=14.9, 7.0 Hz, 1H), 4.21-4.01 (m, 1H), 1.48 (d, J=7.0 Hz, 2H), 1.27 (d, J=6.2 Hz, 3H), 1.26 (d, J=5.8 Hz, 3H).

³¹P NMR (121 MHz, Chloroform-d) δ 8.18 and 7.87.

### Example 32

### Synthesis of 2-chloro-4-nitrophenyl phosphoramidate

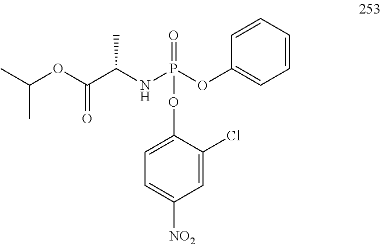 [SMILES: CC(C)OC(=O)[C@H](C)NP(=O)(Oc1ccccc1)Oc1ccc([N+](=O)[O-])cc1Cl]

A solution of phenyl dichlorophosphate (60 g, 42.5 mL, 284 mmol) in dichloromethane (300 mL) was cooled to 0° C. and then treated with (S)-isopropyl 2-aminopropanoate hydrochloride (47.7 g, 284 mmol). The mixture was further cooled to −78° C. and treated dropwise with a solution of triethylamine (57.6 g, 79 mL, 569 mmol) in methylene chloride (300 mL) over a 1 h period. The reaction mixture was warmed to 0° C. for 30 min and then treated with a preformed mixture of 2-chloro-4-nitrophenol (46.9 g, 270 mmol) and triethylamine (28.8 g, 39.6 mL, 284 mmol) in dichloromethane (120 mL) over a 20 min period. After 2 h at 0° C., the mixture was filtered through a fritted funnel, and the collected filtrate concentrated to dryness. The crude gum was dissolved MTBE (500 mL) and washed with 0.2 M K₂CO₃ (2×100 mL) followed by 10% brine (3×75 mL). The organic phase was dried over sodium sulfate, filtered and concentrated to dryness by rotary evaporator to give a diastereomeric mixture (100 g, 93%) as a pale yellow oil.

¹H NMR (400 MHz, Chloroform-d) δ 8.33 (dd, J=2.7, 1.1 Hz, 1H, diastereomer 1), 8.31 (dd, J=2.7, 1.1 Hz, 1H, diastereomer 2), 8.12 (dd, J=9.1, 2.7 Hz, 1H), 7.72 (dt, J=9.1, 1.1 Hz, 1H), 7.40-7.31 (m, 2H), 7.28-7.19 (m, 6H), 5.01 (pd, J=6.3, 5.2 Hz, 1H), 4.22-4.08 (m, 1H), 3.96 (td, J=10.7, 9.1, 3.6 Hz, 1H), 1.43 (dd, J=7.0, 0.6 Hz, 3H), 1.40 (dd, J=7.2, 0.6 Hz, 3H, diastereomer 2), 1.25-1.20 (m, 9H).

### Example 33

### Separation of Compound 253 Diastereomers:

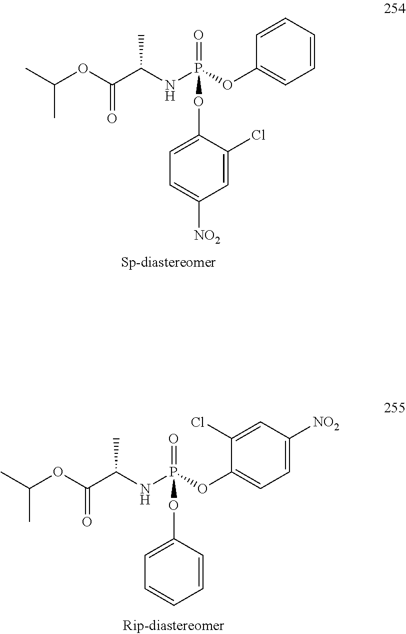 [SMILES: CC(C)OC(=O)[C@H](C)N[P@](=O)(Oc1ccccc1)Oc1ccc([N+](=O)[O-])cc1Cl ; CC(C)OC(=O)[C@H](C)N[P@@](=O)(Oc1ccccc1)Oc1ccc([N+](=O)[O-])cc1Cl]

The diastereomeric mixture 253 (28 g, 63.2 mmol) was dissolved in 2:3 ethyl acetate:hexanes (100 mL) and cooled to −20° C. After 16 h, the resulting white solid was collected by filtration and dried under high vacuum to give a 16:1 Sₚ:Rₚ-diastereomeric mixture (5.5 g, 19.6%). The mother liquor was concentrated and the resulting residue dissolved in 2:3 ethyl acetate:hexanes (50 mL). After 16 h at −10° C., the resulting white solid was collected and dried under high vacuum to give a 1:6 Sₚ:Rₚ-diastereomeric mixture (4 g, 14%). The 16:1 Sₚ:Rₚ-diastereomeric mixture (5.5 g, 12.4 mmol) was suspended in hot hexanes (50 mL) and treated slowly with ethyl acetate (approximately 10 mL) until complete dissolution. After cooling to 0° C., the resulting white solid was collected by filtration, washed with hexanes, and dried under high vacuum to give the Sₚ-diastereomer of 254 (4.2 g, 76%) as a single isomer.

¹H NMR (Sₚ-diastereomer, 400 MHz, Chloroform-d) δ 8.33 (dd, J=2.7, 1.1 Hz, 1H), 8.12 (dd, J=9.1, 2.7 Hz, 1H), 7.71 (dd, J=9.1, 1.2 Hz, 1H), 7.41-7.30 (m, 2H), 7.29-7.11 (m, 3H), 5.00 (m, 1H), 4.25-4.07 (m, 1H), 3.97 (dd, J=12.7, 9.4 Hz, 1H), 1.43 (d, J=7.0 Hz, 3H), 1.23 (d, J=2.2 Hz, 3H), 1.21 (d, J=2.2 Hz, 3H).

The 1:6 Sₚ:Rₚ-diastereomeric mixture (4 g, 12.4 mmol) was suspended in hot hexanes (50 mL) and treated slowly with ethyl acetate (approximately 5 mL) until complete dissolution. After cooling to 0° C., the resulting white solid was collected by filtration, washed with hexanes, and dried under high vacuum to give the Rₚ-diastereomer of 255 (3.2 g, 80%) as a single isomer. Absolute stereochemistry was confirmed by X-ray analysis. ¹H NMR (Rₚ-diastereomer, 400 MHz, Chloroform-d) δ 8.31 (dd, J=2.7, 1.1 Hz, 1H), 8.11 (dd, J=9.1, 2.7 Hz, 1H), 7.72 (dd, J=9.1, 1.2 Hz, 1H), 7.42-7.30 (m, 2H), 7.31-7.14 (m, 3H), 5.01 (p, J=6.3 Hz, 1H), 4.15 (tq, J=9.0, 7.0 Hz, 1H), 4.08-3.94 (m, 1H), 1.40 (d, J=7.0 Hz, 3H), 1.24 (d, J=3.5 Hz, 3H), 1.22 (d, J=3.5 Hz, 3H).

### Example 34

### General Procedure for Phosphoramidate Prodrug Formation:

The desired nucleoside (1 equivalent) to be converted into its 5′-phosphoramidate prodrug was dried in a vacuum oven at 50° C. overnight. The dry nucleoside is placed in a dry flask under an inert atmosphere and suspended in either dry THE or dry DCM to achieve a 0.05M solution. The flask was then cooled to 0° C., and the chlorophosphoramidate reagent (5 equivalents) was added to the suspended nucleoside.

Next, 1-methylimidazole (8 equivalents) was added to the reaction mixture dropwise. The reaction was allowed to stir at room temperature for 12-72 hours. After the reaction was complete as judged by TLC, the reaction mixture was diluted with ethyl acetate. The diluted reaction mixture was then washed with saturated aqueous ammonium chloride solution. The aqueous layer was re-extracted with ethyl acetate. The combined organic layers were then washed with brine, dried over MgSO₄, filtered, and concentrated. The concentrated crude product was then purified on silica eluting with a gradient of DCM to 5% MeOH in DCM.

### Example 35

### General Procedure for Preparation of 5′-Triphosphates:

Nucleoside analogue was dried under high vacuum at 50° C. for 18 h and then dissolved in anhydrous trimethylphosphate (0.3 M). After addition of Proton-Sponge® (1.5 molar equiv), the mixture was cooled to 0° C. and treated dropwise with phosphoryl chloride (1.3 molar equiv) via microsyringe over a 15 min period. The mixture continued stirring at 0° C. for 4 to 6 h while being monitored by tlc (7:2:1 isopropanol: conc. NH₄OH: water). Once greater than 85% conversion to the monophosphate, the reaction mixture was treated with a mixture of bis(tri-n-butylammonium pyrophosphate) (3 molar equiv) and tributylamine (6 molar equiv) in anhydrous DMF (1 mL). After 20 min at 0° C. with monitoring by tlc (11:7:2 NH₄OH: isopropanol: water), the mixture was treated with 20 mL of a 100 mM solution of triethylammonium bicarbonate (TEAB), stirred for 1 h at rt and then extracted with ether (3×15 mL). The aqueous phase was then purified by anion-exchange chromatography over DEAE Sephadex® A-25 resin (11×200 mm) using a buffer gradient from 50 mM (400 mL) to 600 mM (400 mL) TEAB. Fractions of 10 mL were analyzed by tlc (11:7:2 NH₄OH: isopropanol: water). Triphosphate (eluted@500 mM TEAB) containing fractions were combined and concentrated by rotary evaporator (bath <25° C.). The resulting solid was reconstituted in DI water (10 mL) and concentrated by lyophilization.

### Example 36

### Synthesis of (R)-2,2,2-trifluoro-N-(1-hydroxyoctadecan-2-yl)acetamide

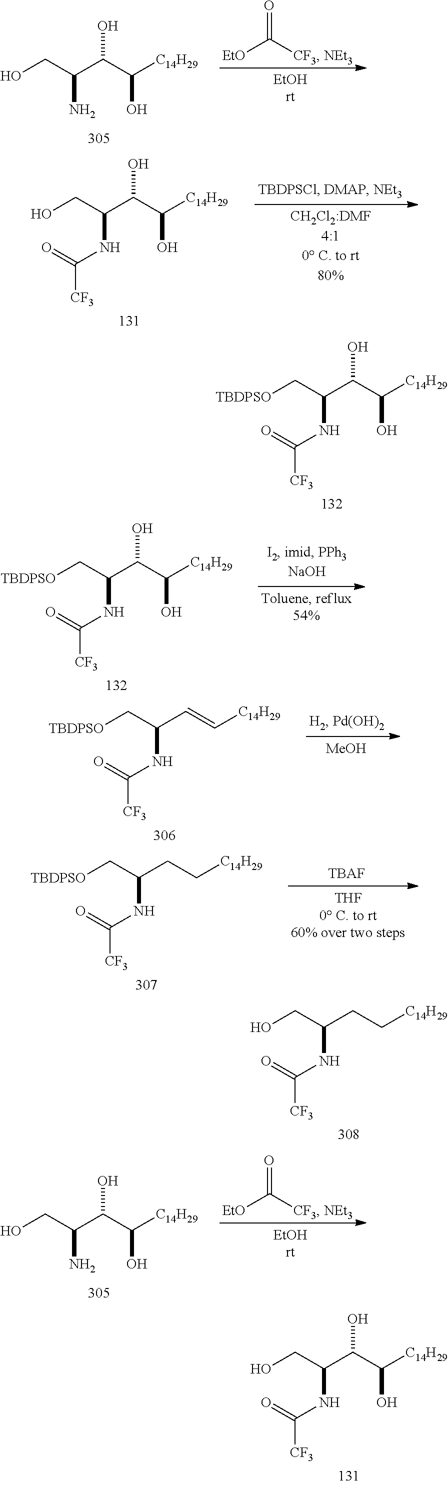

Phytosphingosine (15.75 mmol) was dissolved in EtOH (0.5M) and ethyl trifluoroacetate (15.75 mmol) was added dropwise. NEt₃ (24.41 mmol) was added next the reaction mixture stirred overnight. The solvent was removed in vacuo and the residue was taken up in EtOAc and brine, washed, dried and concentrated. The crude material that was a white powder was good enough to use in the next step without further purification.

Characterization matched literature: Synthesis, 2011, 867.

### Example 37

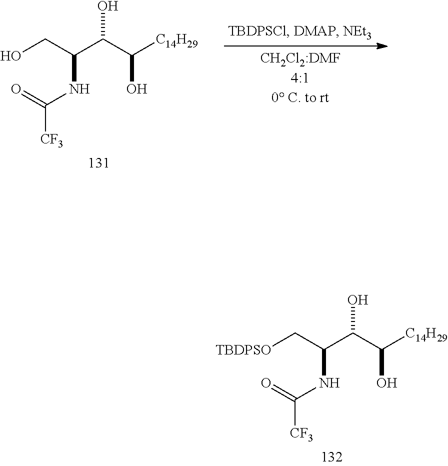

The primary alcohol (15.75 mmol), DMAP (1.575 mmol) and NEt₃ (39.4 mmol) were dissolved in CH₂Cl₂ and DMF (0.18M) mixture and cooled to 0° C. TBDPSCl (19.69 mmol) was added dropwise then the solution was allowed to warm to room temperature and stirred overnight.

NH₄Cl solution was added to quench. The reaction mixture was extracted with EtOAc and the combined organic layers were washed with water (×2) to remove DMF. It was then dried and concentrated. A column was run to purify the mixture. 10-20% EtOAc/Hex. Characterization matched literature: Synthesis, 2011, 867.

### Example 38

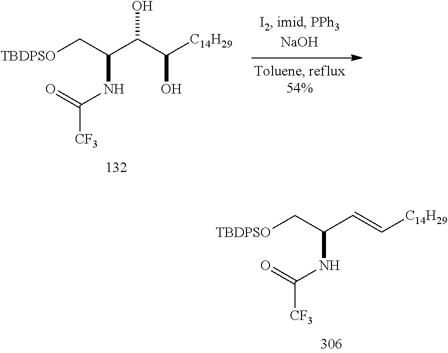

The diol (12.58 mmol), triphenylphosphine (50.3 mmol) and imidazole (50.03 mmol) were dissolved in toluene and reheated to reflux. The iodine (37.7 mmol) was then added slowly and the reaction mixture continued to be stirred at reflux. After three hours it was cooled to room temperature and 1 equivalent of iodine (12.58 mmol) was added followed by 8 equivalents of 1.5M NaOH (100.64 mmol). The reaction mixture was stirred until all the solids dissolved. The aqueous layer was removed in a separatory funnel and the organic layer was washed with Na₂S₂O₃ solution then NaHCO₃ solution then brine. It was dried and concentrated. A column was run to purify the mixture 0-20% EtOAc/Hex and a mixture of cis and trans was obtained but carried on to the next step.

δ ¹H NMR (400 MHz, Chloroform-d) δ 7.64 (ddt, J=7.8, 3.8, 1.7 Hz, 4H), 7.51-7.35 (m, 6H), 6.68 (dd, J=16.0, 8.2 Hz, 1H), 5.6-5.40 (m, 2H), 4.57-4.46 (m, 1H), 3.84-3.62 (m, 2H), 2.04 (q, J=7.0 Hz, 1H), 1.28-1.21 (m, 24H), 1.15-0.98 (m, 9H), 0.90 (t, J=6.8 Hz, 3H).

HRMS: 617.38759.

### Example 3

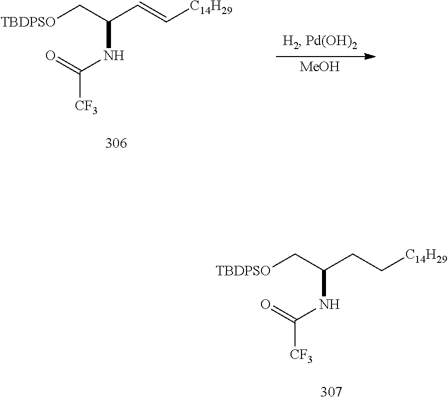

The alkene (2.91 mmol) was dissolved in MeOH (0.1M) and Pd(OH)₂/C (0.146 mmol) was added. A Parr Hydrogenator was used at 40 psi. The palladium catalyst was carefully filtered off through celite and rinsed with EtOAc. The crude material was used in the next step and provided quantitative yield.

### Example 40

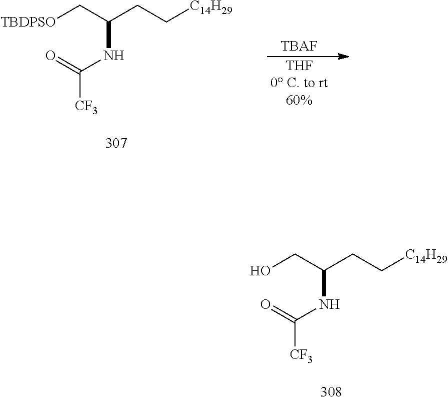

The silyl ether was dissolved in THE and cooled to 0° C. then TBAF was added dropwise. After stirring for 1 hour it was warmed to room temperature. After two hours NH4Cl solution was added and it was extracted with EtOAc, washed with brine and dried and concentrated. A column was run 10-50% EtOAc/Hex.

¹H NMR (400 MHz, Chloroform-d) δ 7.60 (tt, J=7.0, 1.5 Hz, 2H), 7.48-7.33 (m, 4H), 3.73-3.61 (m, 1H), 1.24 (d, J=3.5 Hz, 18H), 1.05 (s, 6H), 0.86 (t, J=6.8 Hz, 3H). HRMS 381.28546.

### Example 41

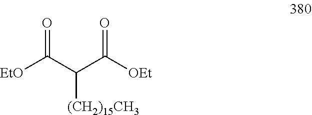

To 33.4 g sodium ethoxide solution (21% wt) in ethanol, diethyl malonate(15 g) and then 1-bromohexadecane (31.5 g) were added dropwise. After reflux for 8 hrs, ethanol was evaporated in vacuo. The remaining suspension was mixed with ice-water(200 ml) and extracted with diethyl ether (3×200 ml). The combined organic layers were dried over MgSO₄, filtered and the filtrate was evaporated in vacuo to yield a viscous oil residue. This residue was purified by column chromatography(silica: 500 g) using hexane/diethyl ether(12:1) as mobile phase to yield the main compound.

### Example 42

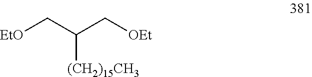

In a 250 mL round-bottomed flask was aluminum lithium hydride (2.503 g, 66.0 mmol) in Diethyl ether (90 ml) to give a suspension. To this suspension was added diethyl 2-hexadecylmalonate (18.12 g, 47.1 mmol) dropwise and the reaction was refluxed for 6 h. The reaction was followed up by TLC using PMA and H2SO4 as drying agents. The excess lithium aluminium hydride was destroyed by 200 ml of ice-water. 150 ml of 10% H2SO4 was added to dissolve aluminium hydrate. The reaction mixture was extracted by diethyl ether (100 ml×3). The organic layer including undissolved product was filtered. The collect solids were washed with ethyl acetate. The filtrate was dried over MgSO₄, filtered and concentrated under reduced pressure. The product was purified on silica (100 g) column eluting with Hexane:EtOAc (3:1) to (1:1).

### Example 43

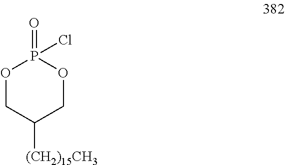

To a solution of 2-hexadecylpropane-1,3-diol (7.04 g, 23.43 mmol) in 100 ml of DCM was added dropwise phosphorous trichloride (3.59 g, 23.43 mmol) dissolved in 20 ml of DCM followed by triethylamine (6.53 ml, 46.9 mmol). The reaction was refluxed for one hour. TLC analysis showed that the starting material was consumed and two new spots formed. The mixture was concentrated to dryness, dissolved in dry diethyl ether and filtered. The filtrate was concentrated to yield the crude product (8.85 g) that was used in the next step without further purification.

### Example 44

Synthesis of 5′-Deuterated Nucleoside Analogs

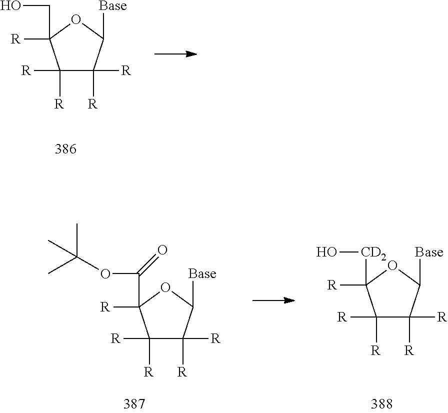

The nucleoside was suspended in methylene chloride (40 mL, partially soluble). After stirring at rt for 30 min the mixture was treated sequentially with PDC, acetic anhydride and then tert-butanol. The mixture continued to stir at room temperature. TLC (5% methanol in DCM) and LCMS indicated only a small amount of remaining starting material at 4 hours. The mixture was filtered through a pad of silica gel that was loaded into a 150 mL fritted funnel. The silica was eluted with ethyl acetate. The collected filtrate was concentrated by under reduced pressure. The crude dark oil was purified by chromatography over silica gel (25 mm×175 mm) with 2:1 hexanes:ethyl acetate to ethyl acetate gradient. The pure fractions were collected and concentrated to give of a white gum.

The material was placed under high vacuum for 2 days and was used in the next step without further purification.

The 5′-protected nucleoside was dissolved in 200 proof ethanol and was then treated with solid sodium borodeuteride. The mixture became homogeneous and was then heated to 80° C. After 12h, a white/pale yellow precipitate formed. The mixture was allowed to cool to rt. TLC (5% methanol in methylene chloride) indicates complete conversion of starting material. The mixture was cooled to 0° C. with an ice-bath and then slowly quenched with acetic acid (approximately 1 mL). The clear solution was warmed to rt and then partitioned between ethyl acetate (30 mL) and brine (3 mL). The organic phase was concentrated and then purified by chromatography over silica gel (19 mm×180 mm) using a mobile phase of 5% methanol in methylene chloride.

### Example 45

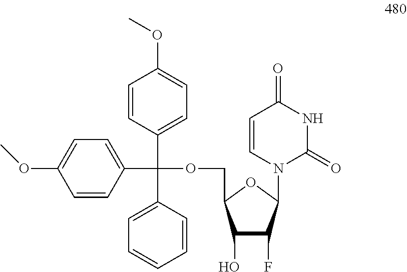

A solution of 2′-deoxy-2′-fluorouridine (6 g, 24.37 mmol) and 4,4′-(chloro(phenyl) methylene)-bis(methoxybenzene) (9.91 g, 29.2 mmol) in pyridine (48.7 ml) was stirred at rt for 16 hours. The mixture was treated with MeOH (20 mL), concentrated to dryness and was partitioned between water (50 mL) and EtOAc (250 mL). The aqueous phase was back extracted with EtOAc (50 mL) and the combined organic layers were washed with water (50 mL) and dried over Na₂SO₄. The solution was concentrated to give 2′-deoxy-2′-fluoro-5′-(4′,4′-dimethoxytrityl)uridine (14 g, quant.) which was used without further purification.

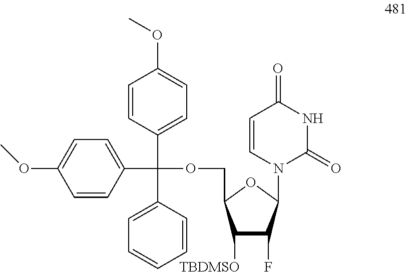

To a solution of 2′-deoxy-2′-fluoro-5′-(4′,4′-dimethoxytrityl)uridine (13.37 g, 24.37 mmol) in methylene chloride (30 mL) were added 1H-imidazole (2.48 g, 36.6 mmol) and tert-butylchlorodimethylsilane (5.51 g, 36.6 mmol). The reaction was stirred for 16 hours and then was diluted with EtOAc (250 mL). The mixture was washed with saturated aqueous sodium bicarbonate (50 mL) and brine (50 mL), dried over Na₂SO4, filtered and concentrated to give 2′-Deoxy-2′-fluoro-3′-O-(tert-butyldimethylsilyl)-5′-(4′,4′-dimethoxytrityl)uridine (16 g, 99%). This product was used in the next step without further purification.

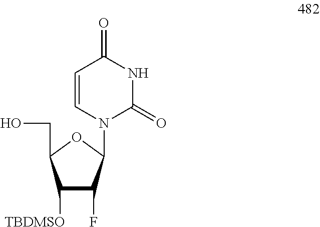

To a solution of 2′-deoxy-2′-fluoro-3′-O-(tert-butyldimethylsilyl)-5′-(4′,4′-dimethoxytrityl) uridine (13.37 g, 20.17 mmol) in DCM (10 mL) were added acetic acid (20.19 ml, 353 mmol) and water (5 ml). The reaction was stirred at room temperature for 20 hours, diluted with EtOAc (250 mL), washed with saturated aqueous NaHCO₃ (2×100 mL) and brine (100 mL), dried (sodium sulfate), filtered and concentrated. The residue was purified by column chromatography over silica gel (1% MeOH in DCM, 2% MeOH in DCM) to afford 2′-deoxy-2′-fluoro-3′-O-(tert-butyldimethylsilyl)uridine (6.73 g, 93% yield) as a yellow solid.

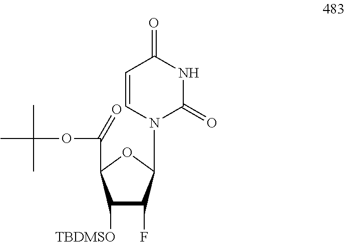

To a suspension of PDC (14.05 g, 37.3 mmol) in anhydrous DCM (37.3 ml)/DMF (9.34 ml) were added sequentially 2-methylpropan-2-ol (35.7 ml, 373 mmol), 2′-deoxy-2′-fluoro-3′-O-(tert-butyldimethylsilyl)uridine (6.73 g, 18.67 mmol) and acetic anhydride (17.62 ml, 187 mmol). After 18 hours, the mixture was quenched with absolute EtOH (5 mL), diluted with EtOAc (15 mL), dried over Na₂SO4, filtered through Celite and concentrated. The crude residue was purified by column chromatography over silica gel using 1% MeOH in DCM to give (2S, 3R, 4R, 5R)-tert-butyl 3-((tert-butyldimethylsilyl)oxy)-5-(2,4-dioxo-3,4-dihydropyrimidin-1(2H)-yl)-4-fluorotetrahydrofuran-2-carboxylate (6.72 g, 83%)

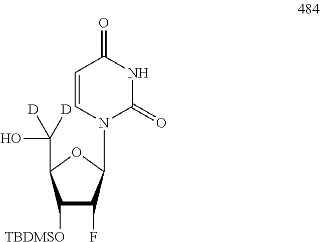

To a solution of (2S, 3R, 4R, 5R)-tert-butyl 3-((tert-butyldimethylsilyl)oxy)-5-(2,4-dioxo-3,4-dihydropyrimidin-1(2H)-yl)-4-fluorotetrahydrofuran-2-carboxylate (3.29 g, 7.64 mmol) was added sodium borodeuteride (1.422 g, 30.6 mmol) in one portion. The reaction was stirred at 80° C. for 20 hours in a sealed tube. The mixture was cooled to room temperature and then quenched with acetic acid (6.99 ml, 122 mmol). The mixture was neutralized with saturated aqueous sodium bicarbonate and extracted with EtOAc. After concentrating, the resulting residue was purified by column chromatography over silica gel (Rf=0.5 hexane EtOAc 1:1) to give [5′-²H2]-2′-deoxy-2′-fluoro-3′-O-(tert-butyldimethylsilyl)uridine (1 g, 36%).

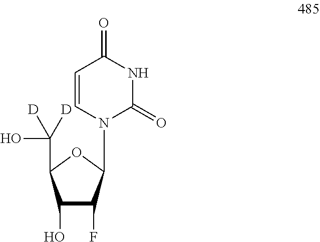

To a solution of [5′-²H2]-2′-deoxy-2′-fluoro-3′-O-(tert-butyldimethylsilyl)uridine (200 mg, 0.552 mmol) in MeOH (6 mL) was added Dowex 50WX8 (H+ form) (6 g) in one portion. The mixture was stirred for 72 h, filtered and concentrated to give [5′-²H2]-2′-deoxy-2′-fluorouridine (150 mg, quant.).

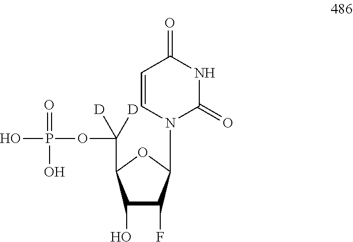

To a solution of phosphoryl trichloride (1.69 mL, 18.13 mmol) in trimethyl phosphate (2 mL) at 5° C., under N2, was added [5′-²H2]-2′-deoxy-2′-fluorouridine (100 mg, 0.403 mmol) in small portions. The solution was stirred vigorously for 2 h at 5° C. and then was quenched by dropwise addition of DI water (8 mL). The reaction mixture was extracted with chloroform (2×10 mL), and the aqueous phase was treated with concentrated with NH40H to pH 6.5, while keeping the solution below 30° C. The aqueous layer was extracted once more with chloroform (10 mL) and then concentrated to dryness. The residue was suspended in MeOH (15 mL), filtered, and concentrated. The resulting solid was purified by column chromatography over silica gel (7:2:1 iPrOH/conc. NH₄OH, H₂O, Rf=0.2). The product was further purified by column chromatography over DEAE using methanol followed by a mobile phase gradient from 0 to 100 mM aqueous ammonium bicarbonate. Fractions were concentrated to dryness, dissolved in water and lyophilized to give [5′-²H₂]-2′-deoxy-2′-fluorouridine-5′-monophosphate (27 mg, 20%) as an amorphous white solid.

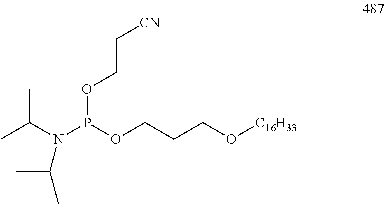

A suspension of 3-hexadecyloxypropan-1-ol (2.02 g, 6.72 mmol) and DIPEA (4.7 mL, 26.9 mmol) in anhydrous methylene chloride (45 mL) was treated dropwise over a 10 minute period with 3-((chloro(diisopropylamino)phosphino)oxy)propanenitrile (3 mL, 13.45 mmol). After 18 hours at room temperature, the mixture was quenched with saturated sodium bicarbonate solution (15 mL) and extracted with ethyl acetate (2×100 mL). Combined organic phases were concentrated to dryness, and the resulting crude residue purified by chromatography over silica gel (25 mm×140 mm) using a solvent gradient from 10 to 20% ethyl acetate in hexanes to give hexadecyloxypropyl-(2-cyanoethyl) diisopropylphosphoramidite (2.1 g, 65%) as a white solid.

¹H NMR (400 MHz, Chloroform-d) δ 3.89-3.54 (m, 6H), 3.49 (t, J=6.3 Hz, 2H), 3.39 (t, J=6.7 Hz, 2H), 2.64 (t, J=6.6 Hz, 2H), 1.87 (p, J=6.3 Hz, 2H), 1.57 (p, J=6.3 Hz, 2H), 1.25 (s, 26H), 1.18 (dd, J=6.8, 3.5 Hz, 12H), 0.87 (t, J=6.6 Hz, 3H).

³¹P NMR (162 MHz, Chloroform-d) δ 147.40.

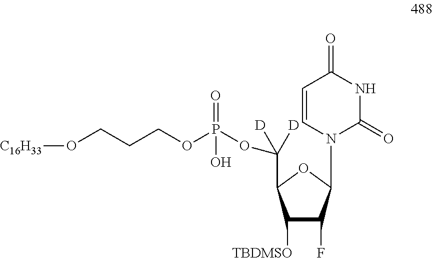

A solution of [5′-²H₂]-2′-deoxy-2-fluoro-3′-O-(tert-butyldimethylsilyl)uridine (600 mg, 1.65 mmol) and hexadecyloxypropyl-(2-cyanoethyl) diisopropylphosphoramidite (1.65 g, 3.31 mmol) in anhydrous THE (22 mL) was treated dropwise with 1-H-tetrazole (14.7 mL of 0.45 M solution in acetonitrile, 6.62 mmol). After 16 hours at room temperature, the mixture was treated dropwise with tert-butyl hydroperoxide (1.5 mL of a 5.5 M solution in nonane, 8.28 mmol) and stirred at room temperature for 1 hour and then quenched with 1.0 M aqueous solution of sodium thiosulfate (40 mL). After 30 min, the mixture was extracted with ethyl acetate (2×80 mL). Combined organic phases were washed with brine (40 mL) and dried over sodium sulfate, filtered, and concentrated. The resulting residue was purified by column chromatography over silica gel (40 g) with a mobile phase gradient from 1% to 5% methanol in methylene chloride to give the cyanoethyl phosphate intermediate which without further purification was dissolved in methanol (30 mL) and treated with concentrated ammonium hydroxide (5 mL, 128 mmol). After 4 hours at room temperature, the mixture was concentrated to dryness. The resulting residue was purified by column chromatography over silica gel using a CombiFlash instrument equipped with a 40 g silica cartridge eluting with a solvent gradient from 5 to 25% methanol in methylene chloride to give [5′-²H2]-2′-deoxy-2′-fluoro-3′-O-(tert-butyldimethylsilyl)-5′-((hexadecyloxypropyl) phospho)uridine (1 g, 82%) as a white foam.

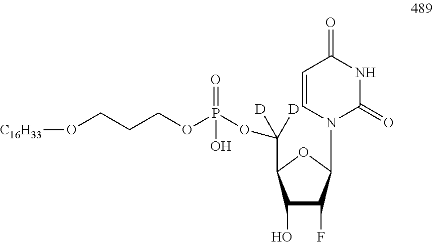

A solution of [5′-²H2]-2′-deoxy-2′-fluoro-3′-O-(tert-butyldimethylsilyl)-5′-((hexadecyloxypropyl) phospho)uridine (1 g, 1.38 mmol) in THE (15 mL) was treated with acetic acid (0.5 g, 8.28 mmol) and triethylammonium fluoride (1.2 g, 5.52 mmol). After 36 hours, the mixture was concentrated and the resulting residue eluted through a short column (11 mm×90 mm) of Dowex 50WX8 (H+form) using methanol (120 mL) as the mobile phase. The product was further purified by column chromatography over silica gel (24 g) using a mobile phase gradient from 0 to 25% methanol in methylene chloride with 2.5% (v/v) ammonium hydroxide. Pure fractions were pooled and concentrated. The resulting solid was co-evaporated with methylene chloride (2×75 mL) and then dried under high vacuum for 19 hours to give [5′-²H2]-2′-deoxy-2′-fluoro-5′-((hexadecyloxypropyl)phospho)-uridine (455 mg, 54%) as a white solid. 1H NMR (400 MHz, Chloroform-d4/Methanol-d₄) δ 7.75 (d, J=8.1 Hz, 1H), 5.95 (dd, J=17.9, 1.6 Hz, 1H), 5.70 (d, J=8.1 Hz, 1H), 5.01 (ddd, J=52.8, 4.6, 1.7 Hz, 1H), 4.30 (ddd, J=20.7, 8.1, 4.5 Hz, 1H), 4.16-4.07 (m, 3H), 3.51 (t, J=6.2 Hz, 2H), 3.41 (t, J=6.7 Hz, 2H), 1.92 (p, J=7.6 Hz, 2H), 1.53 (p, J=7.6 Hz, 2H), 1.25 (s, 26H), 0.87 (d, J=7.6 Hz, 3H).

¹³C NMR (101 MHz, Chloroform-d4/Methanol-d4) δ 164.31, 150.24, 140.33, 102.11, 94.19, 92.32, 88.88, 88.53, 80.83, 80.75, 71.18, 67.62, 67.45, 66.50, 66.40, 64.83, 64.77, 63.81, 31.81, 30.37, 30.29, 29.59, 29.57, 29.54, 29.51, 29.47, 29.41, 29.25, 26.00, 25.96, 22.57, 13.96.

³¹P NMR (162 MHz, Chloroform-d4/Methanol-d4) δ −0.87.

HRMS C₂₈H₄₉D₂FN₂O₉P [M+H⁺]; calculated: 611.34359, found: 611.34363.

### Example 46

### Assay Protocols

### (1) Screening Assays for DENV, JEV, POWV, WNV, YFV, PTV, RVFV, CHIKV, EEEV, VEEV, WEEV, TCRV, PCV, JUNV, MPRLV

Primary cytopathic effect (CPE) reduction assay. Four-concentration CPE inhibition assays are performed. Confluent or near-confluent cell culture monolayers in 96-well disposable microplates are prepared. Cells are maintained in MEM or DMEM supplemented with FBS as required for each cell line. For antiviral assays the same medium is used but with FBS reduced to 2% or less and supplemented with 50 μg/ml gentamicin. The test compound is prepared at four log₁₀ final concentrations, usually 0.1, 1.0, 10, and 100 μg/ml or μM. The virus control and cell control wells are on every microplate. In parallel, a known active drug is tested as a positive control drug using the same method as is applied for test compounds. The positive control is tested with each test run. The assay is set up by first removing growth media from the 96-well plates of cells. Then the test compound is applied in 0.1 ml volume to wells at 2× concentration. Virus, normally at <100 50% cell culture infectious doses (CCID₅₀) in 0.1 ml volume, is placed in those wells designated for virus infection. Medium devoid of virus is placed in toxicity control wells and cell control wells. Virus control wells are treated similarly with virus. Plates are incubated at 37° C. with 5% CO₂ until maximum CPE is observed in virus control wells. The plates are then stained with 0.011% neutral red for approximately two hours at 37° C. in a 5% CO₂ incubator. The neutral red medium is removed by complete aspiration, and the cells may be rinsed 1× with phosphate buffered solution (PBS) to remove residual dye. The PBS is completely removed and the incorporated neutral red is eluted with 50% Sorensen's citrate buffer/50% ethanol (pH 4.2) for at least 30 minutes. Neutral red dye penetrates into living cells, thus, the more intense the red color, the larger the number of viable cells present in the wells. The dye content in each well is quantified using a 96-well spectrophotometer at 540 nm wavelength. The dye content in each set of wells is converted to a percentage of dye present in untreated control wells using a Microsoft Excel computer-based spreadsheet. The 50% effective (EC₅₀, virus-inhibitory) concentrations and 50% cytotoxic (CC₅₀, cell-inhibitory) concentrations are then calculated by linear regression analysis. The quotient of CC₅₀ divided by EC₅₀ gives the selectivity index (SI) value.

Secondary CPE/Virus yield reduction (VYR) assay. This assay involves similar methodology to what is described in the previous paragraphs using 96-well microplates of cells. The differences are noted in this section. Eight half-log₁₀ concentrations of inhibitor are tested for antiviral activity and cytotoxicity. After sufficient virus replication occurs, a sample of supernatant is taken from each infected well (three replicate wells are pooled) and held for the VYR portion of this test, if needed. Alternately, a separate plate may be prepared and the plate may be frozen for the VYR assay. After maximum CPE is observed, the viable plates are stained with neutral red dye. The incorporated dye content is quantified as described above. The data generated from this portion of the test are neutral red EC₅₀, CC₅₀, and SI values. Compounds observed to be active above are further evaluated by VYR assay. The VYR test is a direct determination of how much the test compound inhibits virus replication. Virus that was replicated in the presence of test compound is titrated and compared to virus from untreated, infected controls. Titration of pooled viral samples (collected as described above) is performed by endpoint dilution. This is accomplished by titrating log₁₀ dilutions of virus using 3 or 4 microwells per dilution on fresh monolayers of cells by endpoint dilution. Wells are scored for presence or absence of virus after distinct CPE (measured by neutral red uptake) is observed. Plotting the log₁₀ of the inhibitor concentration versus log₁₀ of virus produced at each concentration allows calculation of the 90% (one log₁₀) effective concentration by linear regression. Dividing EC₉₀ by the CC₅₀ obtained in part 1 of the assay gives the SI value for this test.

### Example 47

### (2) Screening Assays for Lassa Fever Virus (LASV)

Primary Lassa fever virus assay. Confluent or near-confluent cell culture monolayers in 12-well disposable cell culture plates are prepared. Cells are maintained in DMEM supplemented with 10% FBS. For antiviral assays the same medium is used but with FBS reduced to 2% or less and supplemented with 1% penicillin/streptomycin. The test compound is prepared at four log₁₀ final concentrations, usually 0.1, 1.0, 10, and 100 μg/ml or μM. The virus control and cell control will be run in parallel with each tested compound. Further, a known active drug is tested as a positive control drug using the same experimental set-up as described for the virus and cell control. The positive control is tested with each test run. The assay is set up by first removing growth media from the 12-well plates of cells, and infecting cells with 0.01 MOI of LASV strain Josiah. Cells will be incubated for 90 min: 500 μl inoculum/M12 well, at 37° C., 5% CO₂ with constant gentle rocking. The inoculums will be removed and cells will be washed 2× with medium. Then the test compound is applied in 1 ml of total volume of media. Tissue culture supernatant (TCS) will be collected at appropriate time points. TCS will then be used to determine the compounds inhibitory effect on virus replication. Virus that was replicated in the presence of test compound is titrated and compared to virus from untreated, infected controls. For titration of TCS, serial ten-fold dilutions will be prepared and used to infect fresh monolayers of cells. Cells will be overlaid with 1% agarose mixed 1:1 with 2×MEM supplemented with 10% FBS and 1% penecillin, and the number of plaques determined. Plotting the log₁₀ of the inhibitor concentration versus log₁₀ of virus produced at each concentration allows calculation of the 90% (one log₁₀) effective concentration by linear regression.

Secondary Lassa fever virus assay. The secondary assay involves similar methodology to what is described in the previous paragraphs using 12-well plates of cells. The differences are noted in this section. Cells are being infected as described above but this time overlaid with 1% agarose diluted 1:1 with 2×MEM and supplemented with 2% FBS and 1% penicillin/streptomycin and supplemented with the corresponding drug concentration. Cells will be incubated at 37° C. with 5% CO₂ for 6 days. The overlay is then removed and plates stained with 0.05% crystal violet in 10% buffered formalin for approximately twenty minutes at room temperature. The plates are then washed, dried and the number of plaques counted. The number of plaques is in each set of compound dilution is converted to a percentage relative to the untreated virus control. The 50% effective (EC50, virus-inhibitory) concentrations are then calculated by linear regression analysis.

### Example 48

### (3) Screening Assays for Ebola Virus (EBOV) and Nipah Virus (NIV)

Primary Ebola/Nipah virus assay. Four-concentration plaque reduction assays are performed. Confluent or near-confluent cell culture monolayers in 12-well disposable cell culture plates are prepared. Cells are maintained in DMEM supplemented with 10% FBS. For antiviral assays the same medium is used but with FBS reduced to 2% or less and supplemented with 1% penicillin/streptomycin. The test compound is prepared at four log₁₀ final concentrations, usually 0.1, 1.0, 10, and 100 μg/ml or μM. The virus control and cell control will be run in parallel with each tested compound. Further, a known active drug is tested as a positive control drug using the same experimental set-up as described for the virus and cell control. The positive control is tested with each test run. The assay is set up by first removing growth media from the 12-well plates of cells. Then the test compound is applied in 0.1 ml volume to wells at 2× concentration. Virus, normally at approximately 200 plaque-forming units in 0.1 ml volume, is placed in those wells designated for virus infection. Medium devoid of virus is placed in toxicity control wells and cell control wells. Virus control wells are treated similarly with virus. Plates are incubated at 37° C. with 5% CO₂ for one hour. Virus-compound inoculums will be removed, cells washed and overlaid with 1.6% tragacanth diluted 1:1 with 2×MEM and supplemented with 2% FBS and 1% penicillin/streptomycin and supplemented with the corresponding drug concentration. Cells will be incubated at 37° C. with 5% CO₂ for 10 days. The overlay is then removed and plates stained with 0.05% crystal violet in 10% buffered formalin for approximately twenty minutes at room temperature. The plates are then washed, dried and the number of plaques counted. The number of plaques is in each set of compound dilution is converted to a percentage relative to the untreated virus control. The 50% effective (EC₅₀, virus-inhibitory) concentrations are then calculated by linear regression analysis.

Secondary Ebola/NIpah virus assay with VYR component. The secondary assay involves similar methodology to what is described in the previous paragraphs using 12-well plates of cells. The differences are noted in this section. Eight half-log₁₀ concentrations of inhibitor are tested for antiviral activity. One positive control drug is tested per batch of compounds evaluated. For this assay, cells are infected with virus. Cells are being infected as described above but this time incubated with DMEM supplemented with 2% FBS and 1% penicillin/streptomycin and supplemented with the corresponding drug concentration. Cells will be incubated for 10 days at 37° C. with 5% CO₂, daily observed under microscope for the number of green fluorescent cells. Aliquots of supernatant from infected cells will be taken daily and the three replicate wells are pooled. The pooled supernatants are then used to determine the compounds inhibitory effect on virus replication. Virus that was replicated in the presence of test compound is titrated and compared to virus from untreated, infected controls. For titration of pooled viral samples, serial ten-fold dilutions will be prepared and used to infect fresh monolayers of cells. Cells are overlaid with tragacanth and the number of plaques determined. Plotting the log₁₀ of the inhibitor concentration versus log₁₀ of virus produced at each concentration allows calculation of the 90% (one log₁₀) effective concentration by linear regression.

### Example 49

### Anti-Dengue Virus Cytoprotection Assay:

Cell Preparation—BHK21 cells (Syrian golden hamster kidney cells, ATCC catalog #CCL-I 0), Vero cells (African green monkey kidney cells, ATCC catalog #CCL-81), or Huh-7 cells (human hepatocyte carcinoma) were passaged in DMEM supplemented with 10% FBS, 2 mM L-glutamine, 100 U/mL penicillin, and 100 μg/mL streptomycin in T-75 flasks prior to use in the antiviral assay. On the day preceding the assay, the cells were split 1:2 to assure they were in an exponential growth phase at the time of infection. Total cell and viability quantification was performed using a hemocytometer and Trypan Blue dye exclusion. Cell viability was greater than 95% for the cells to be utilized in the assay. The cells were resuspended at 3×10³ (5×10⁵ for Vero cells and Huh-7 cells) cells per well in tissue culture medium and added to flat bottom microtiter plates in a volume of 100 μL. The plates were incubated at 37° C./5% CO₂ overnight to allow for cell adherence. Monolayers were observed to be approximately 70% confluent.

Virus Preparation—The Dengue virus type 2 New Guinea C strain was obtained from ATCC (catalog #VR-1584) and was grown in LLC-MK2 (Rhesus monkey kidney cells; catalog #CCL-7.1) cells for the production of stock virus pools. An aliquot of virus pretitered in BHK21 cells was removed from the freezer (−80° C.) and allowed to thaw slowly to room temperature in a biological safety cabinet. Virus was resuspended and diluted into assay medium (DMEM supplemented with 2% heat-inactivated FBS, 2 mM L-glutamine, 100 U/mL penicillin, and 100 μg/mL streptomycin) such that the amount of virus added to each well in a volume of 100 μL was the amount determined to yield 85 to 95% cell killing at 6 days post-infection.

Plate Format—Each plate contains cell control wells (cells only), virus control wells (cells plus virus), triplicate drug toxicity wells per compound (cells plus drug only), as well as triplicate experimental wells (drug plus cells plus virus).

Efficacy and Toxicity XTT-Following incubation at 37° C. in a 5% CO₂ incubator, the test plates were stained with the tetrazolium dye XTT (2,3-bis(2-methoxy-4-nitro-5-sulfophenyl)-5-[(phenylamino)carbonyl]-2H-tetrazolium hydroxide). XTT-tetrazolium was metabolized by the mitochondrial enzymes of metabolically active cells to a soluble formazan product, allowing rapid quantitative analysis of the inhibition of virus-induced cell killing by antiviral test substances. XTT solution was prepared daily as a stock of 1 mg/mL in RPMI 1640. Phenazine methosulfate (PMS) solution was prepared at 0.15 mg/mL in PBS and stored in the dark at −20° C. XTT/PMS stock was prepared immediately before use by adding 40 μL of PMS per ml of XTT solution. Fifty microliters of XTT/PMS was added to each well of the plate and the plate was reincubated for 4 hours at 37° C. Plates were sealed with adhesive plate sealers and shaken gently or inverted several times to mix the soluble formazan product and the plate was read spectrophotometrically at 450/650 nm with a Molecular Devices Vmax plate reader.

Data Analysis —Raw data was collected from the Softmax Pro 4.6 software and imported into a Microsoft Excel spreadsheet for analysis. The percent reduction in viral cytopathic effect compared to the untreated virus controls was calculated for each compound. The percent cell control value was calculated for each compound comparing the drug treated uninfected cells to the uninfected cells in medium alone.

### Example 50

### Anti-RSV Cytoprotection Assay:

Cell Preparation-HEp2 cells (human epithelial cells, A TCC catalog #CCL-23) were passaged in DMEM supplemented with 10% FBS, 2 mM L-glutamine, 100 U/mL penicillin, 100 μg/mL streptomycin 1 mM sodium pyruvate, and 0.1 mM NEAA, T-75 flasks prior to use in the antiviral assay. On the day preceding the assay, the cells were split 1:2 to assure they were in an exponential growth phase at the time of infection. Total cell and viability quantification was performed using a hemocytometer and Trypan Blue dye exclusion. Cell viability was greater than 95% for the cells to be utilized in the assay. The cells were resuspended at 1×10⁴ cells per well in tissue culture medium and added to flat bottom microtiter plates in a volume of 100 μL. The plates were incubated at 37° C./5% C02 overnight to allow for cell adherence. Virus Preparation—The RSV strain Long and RSV strain 9320 were obtained from ATCC (catalog #VR-26 and catalog #VR-955, respectively) and were grown in HEp2 cells for the production of stock virus pools. A pretitered aliquot of virus was removed from the freezer (−80° C.) and allowed to thaw slowly to room temperature in a biological safety cabinet. Virus was resuspended and diluted into assay medium (DMEM supplemented with 2% heat-inactivated FBS, 2 mM L-glutamine, 100 U/mL penicillin, 100 μg/mL streptomycin, 1 mM sodium pyruvate, and 0.1 mM NEAA) such that the amount of virus added to each well in a volume of 100 μL was the amount determined to yield 85 to 95% cell killing at 6 days post-infection. Efficacy and Toxicity XTT-Plates were stained and analyzed as previously described for the Dengue cytoprotection assay.

### Example 51

### Anti-Influenza Virus Cytoprotection Assay:

Cell Preparation-MOCK cells (canine kidney cells, ATCC catalog #CCL-34) were passaged in DMEM supplemented with 10% FBS, 2 mM L-glutamine, 100 U/mL penicillin, 100 μg/mL streptomycin 1 mM sodium pyruvate, and 0.1 mM NEAA, T-75 flasks prior to use in the antiviral assay. On the day preceding the assay, the cells were split 1:2 to assure they were in an exponential growth phase at the time of infection. Total cell and viability quantification was performed using a hemocytometer and Trypan Blue dye exclusion. Cell viability was greater than 95% for the cells to be utilized in the assay. The cells were resuspended at 1×10⁴ cells per well in tissue culture medium and added to flat bottom microtiter plates in a volume of 100 μL. The plates were incubated at 37° C./5% C02 overnight to allow for cell adherence.

Virus Preparation—The influenza A/PR/8/34 (A TCC #VR-95), A/CA/05/09 (CDC), A/NY/18/09 (CDC) and A/NWS/33 (ATCC #VR-219) strains were obtained from ATCC or from the Center of Disease Control and were grown in MDCK cells for the production of stock virus pools. A pretitered aliquot of virus was removed from the freezer (−80° C.) and allowed to thaw slowly to room temperature in a biological safety cabinet. Virus was resuspended and diluted into assay medium (DMEM supplemented with 0.5% BSA, 2 mM L-glutamine, 100 U/mL penicillin, 100 μg/mL streptomycin, 1 mM sodium pyruvate, 0.1 mM NEAA, and 1 μg/ml TPCK-treated trypsin) such that the amount of virus added to each well in a volume of 100 μL was the amount determined to yield 85 to 95% cell killing at 4 days post-infection. Efficacy and Toxicity XTT-Plates were stained and analyzed as previously described for the Dengue cytoprotection assay.

### Example 52

### Anti-Hepatitis C Virus Assay:

Cell Culture—The reporter cell line Huh-luc/neo-ET was obtained from Dr. Ralf Bartenschlager (Department of Molecular Virology, Hygiene Institute, University of Heidelberg, Germany) by ImQuest BioSciences through a specific licensing agreement. This cell line harbors the persistently replicating I₃₈₉1uc-ubi-neo/NS3-3′/ET replicon containing the firefly luciferase gene-ubiquitin-neomycin phosphotransferase fusion protein and EMCV IRES driven NS3-5B HCV coding sequences containing the ET tissue culture adaptive mutations (E1202G, T12081, and K1846T). A stock culture of the Huh-luc/neo-ET was expanded by culture in DMEM supplemented with I 0% FCS, 2 mM glutamine, penicillin (100 μU/mL)/streptomycin (100 μg/mL) and 1× nonessential amino acids plus 1 mg/mL G418. The cells were split 1:4 and cultured for two passages in the same media plus 250 μg/mL G418. The cells were treated with trypsin and enumerated by staining with trypan blue and seeded into 96-well tissue culture plates at a cell culture density 7.5×10³ cells per well and incubated at 37° C. 5% CO₂ for 24 hours. Following the 24 hour incubation, media was removed and replaced with the same media minus the G418 plus the test compounds in triplicate. Six wells in each plate received media alone as a no-treatment control. The cells were incubated an additional 72 hours at 37° C. 5% CO₂ then anti-HCV activity was measured by luciferase endpoint. Duplicate plates were treated and incubated in parallel for assessment of cellular toxicity by XTT staining.

Cellular Viability—The cell culture monolayers from treated cells were stained with the tetrazolium dye XTT to evaluate the cellular viability of the Huh-luc/neo-ET reporter cell line in the presence of the compounds.

Measurement of Virus Replication-HCV replication from the replicon assay system was measured by luciferase activity using the britelite plus luminescence reporter gene kit according to the manufacturer's instructions (Perkin Elmer, Shelton, CT). Briefly, one vial of britelite plus lyophilized substrate was solubilized in 10 mL of britelite reconstitution buffer and mixed gently by inversion. After a 5 minute incubation at room temperature, the britelite plus reagent was added to the 96 well plates at 100 μL per well. The plates were sealed with adhesive film and incubated at room temperature for approximately 10 minutes to lyse the cells. The well contents were transferred to a white 96-well plate and luminescence was measured within 15 minutes using the Wallac 1450Microbeta Trilux liquid scintillation counter. The data were imported into a customized Microsoft Excel 2007 spreadsheet for determination of the 50% virus inhibition concentration (EC₅₀).

### Example 53

### Anti-Parainfluenza-3 Cytoprotection Assay:

Cell Preparation—HEp2 cells (human epithelial cells, ATCC catalog #CCL-23) were passaged in DMEM supplemented with 10% FBS, 2 mM L-glutamine, 100 U/mL penicillin, 100 μg/mL streptomycin 1 mM sodium pyruvate, and 0.1 mM NEAA, T-75 flasks prior to use in the antiviral assay. On the day preceding the assay, the cells were split 1:2 to assure they were in an exponential growth phase at the time of infection. Total cell and viability quantification was performed using a hemocytometer and Trypan Blue dye exclusion. Cell viability was greater than 95% for the cells to be utilized in the assay. The cells were resuspended at 1×10⁴cells per well in tissue culture medium and added to flat bottom microtiter plates in a volume of 100 μL. The plates were incubated at 37° C./5% C02 overnight to allow for cell adherence.

Virus Preparation—The Parainfluenza virus type 3 SF4 strain was obtained from ATCC (catalog #VR-281) and was grown in HEp2 cells for the production of stock virus pools. A pretitered aliquot of virus was removed from the freezer (−80° C.) and allowed to thaw slowly to room temperature in a biological safety cabinet. Virus was resuspended and diluted into assay medium (DMEM supplemented with 2% heat-inactivated FBS, 2 mM L-glutamine, 100 U/mL penicillin, and 100 μg/mL streptomycin) such that the amount of virus added to each well in a volume of 100 μL was the amount determined to yield 85 to 95% cell killing at 6 days post-infection.

Plate Format—Each plate contains cell control wells (cells only), virus control wells (cells plus virus), triplicate drug toxicity wells per compound (cells plus drug only), as well a triplicate experimental wells (drug plus cells plus virus). Efficacy and Toxicity XTT-Following incubation at 37° C. in a 5% CO₂ incubator, the test plates were stained with the tetrazolium dye XTT (2,3-bis(2-methoxy-4-nitro-5-sulfophenyl)-5-[(phenylamino)carbonyl]-2H-tetrazol hydroxide). XTT-tetrazolium was metabolized by the mitochondrial enzymes of metabolically active cells to a soluble formazan product, allowing rapid quantitative analysis of the inhibition of virus-induced cell killing by antiviral test substances. XTT solution was prepared daily as a stock of 1 mg/mL in RPMI1640. Phenazine methosulfate (PMS) solution was prepared at 0.15 mg/mL in PBS and stored in the dark at −20° C. XTT/PMS stock was prepared immediately before use by adding 40 μL of PMS per ml of XTT solution. Fifty microliters of XTT/PMS was added to each well of the plate and the plate was reincubated for 4 hours at 37° C. Plates were sealed with adhesive plate sealers and shaken gently or inverted several times to mix the soluble fomlazan product and the plate was read spectrophotometrically at 450/650 nm with a Molecular Devices Vmax plate reader.

Data Analysis−Raw data was collected from the Softmax Pro 4.6 software and imported into a Microsoft Excel spreadsheet for analysis. The percent reduction in viral cytopathic effect compared to the untreated virus controls was calculated for each compound. The percent cell control value was calculated for each compound comparing the drug treated uninfected cells to the uninfected cells in medium alone.

### Example 54

### Influenza Polymerase Inhibition Assay:

Virus Preparation—Purified influenza virus A/PR/8/34 (1 ml) was obtained from Advanced Biotechnologies, Inc. (Columbia, MD), thawed and dispensed into five aliquots for storage at −80° C. until use. On the day of assay set up, 20 μL of 2.5% Triton N-101 was added to 180 μL of purified virus. The disrupted virus was diluted 1:2 in a solution containing 0.25% Triton and PBS. Disruption provided the source of influenza ribonucleoprotein (RNP) containing the influenza RNA-dependent RNA polymerase and template RNA. Samples were stored on ice until use in the assay.

Polymerase reaction—Each 50 μL polymerase reaction contained the following: 5 μL of the disrupted RNP, 100 mM Tris-HCl (pH 8.0), 100 mM KCl, 5 mM MgCl₂. 1 mM dithiothreitol, 0.25% Triton N-101, 5 μCi of [α-³²P]GTP, 100 μM ATP, 50 μM each (CTP, UTP), 1 μM GTP, and 200 μM adenyl (3′-5′) guanosine. For testing the inhibitor, the reactions contained the inhibitor and the same was done for reactions containing the positive control (2′-Deoxy-2′-fluoroguanosine-5′-triphosphate). Other controls included RNP+reaction mixture, and RNP+I % DMSO. The reaction mixture without the ApG primer and NTPs was incubated at 30° C. for 20 minutes. Once the ApG and NTPs were added to the reaction mixture, the samples were incubated at 30° C. for 1 hour then immediately followed by the transfer of the reaction onto glass-fiber filter plates and subsequent precipitation with 10% trichloroacetic acid (TCA). The plate was then washed five times with 5% TCA followed by one wash with 95% ethanol. Once the filter had dried, incorporation of [a-³²P]GTP was measured using a liquid scintillation counter (Micro beta).

Plate Format—Each test plate contained triplicate samples of the three compounds (6 concentrations) in addition to triplicate samples of RNP+reaction mixture (RNP alone), RNP+1% DMSO, and reaction mixture alone (no RNP).

Data Analysis—Raw data was collected from the Micro Beta scintillation counter.

The incorporation of radioactive GTP directly correlates with the levels of polymerase activity. The “percent inhibition values” were obtained by dividing the mean value of each test compound by the RNP+1% DMSO control. The mean obtained at each concentration of 2DFGTP was compared to the RNP+reaction control. The data was then imported into Microsoft Excel spreadsheet to calculate the IC₅₀ values by linear regression analysis.

### Example 55

### HCV Polymerase Inhibition Assay:

Activity of compounds for inhibition of HCV polymerase was evaluated using methods previously described (Lam eta!. 2010. Antimicrobial Agents and Chemotherapy 54(8):3187-3196). HCV NS5B polymerase assays were performed in 20 μL volumes in 96 well reaction plates. Each reaction contained 40 ng/μL purified recombinant NS5BA22 genotype-1b polymerase, 20 ng/μL of HCV genotype-1b complimentary IRES template, 1 μM of each of the four natural ribonucleotides, 1 U/mL Optizyme RNAse inhibitor (Promega, Madison, WI), 1 mM MgCl₂, 0.75 mM MnCl₂, and 2 mM dithiothreitol (DTT) in 50 mM HEPES buffer (pH 7.5). Reaction mixtures were assembled on ice in two steps. Step 1 consisted of combining all reaction components except the natural nucleotides and labeled UTP in a polymerase reaction mixture. Ten microliters (10 μL) of the polymerase mixture was dispensed into individual wells of the 96 well reaction plate on ice. Polymerase reaction mixtures without NS5B polymerase were included as no enzyme controls. Serial half-logarithmic dilutions of test and control compounds, 2′-O-Methyl-CTP and 2′-O-Methyl-GTP (Trilink, San Diego, CA), were prepared in water and 5 μL of the serial diluted compounds or water alone (no compound control) were added to the wells containing the polymerase mixture. Five microliters of nucleotide mix (natural nucleotides and labeled UTP) was then added to the reaction plate wells and the plate was incubated at 27° C. for 30 minutes. The reactions were quenched with the addition of 80 μL stop solution (12.5 mM EDTA, 2.25 M NaCl, and 225 mM sodium citrate) and the RNA products were applied to a Hybond-N+ membrane (GE Healthcare, Piscataway, N.J) under vacuum pressure using a dot blot apparatus. The membrane was removed from the dot blot apparatus and washed four times with 4×SSC (0.6 M NaCl, and 60 mM sodium citrate), and then rinsed one time with water and once with 100% ethanol. The membrane was air dried and exposed to a phosphoimaging screen and the image captured using a Typhoon 8600 Phospho imager. Following capture of the image, the membrane was placed into a Micro beta cassette along with scintillation fluid and the CPM in each reaction was counted on a Micro beta 1450.

CPM data were imported into a custom Excel spreadsheet for determination of compound IC₅₀s.

### Example 56

### NS5B RNA-Dependent RNA Polymerase Reaction Conditions

Compounds were assayed for inhibition of NS5B-621 from HCV GT-1b Con-1. Reactions included purified recombinant enzyme, 1 u/μL negative-strand HCV IRES RNA template, and 1 μM NTP substrates including either [³²P]-CTP or [³²P]-UTP. Assay plates were incubated at 27° C. for 1 hour before quench. [3²P]incorporation into macromolecular product was assessed by filter binding.

### Example 57

### Human DNA Polymerase Inhibition Assay:

The human DNA polymerase alpha (catalog #1075), beta (catalog #1077), and gamma (catalog #1076) were purchased from CHIMERx (Madison, WI). Inhibition of beta and gamma DNA polymerase activity was assayed in microtiter plates in a 50 uL reaction mixture containing 50 mM Tris-HCl (pH 8.7), KCl (10 mM for beta and 100 mM for gamma), 10 mM MgCl₂, 0.4 mg/mL BSA, 1 mM DTT, 15% glycerol, 0.05 mM of dCTP, dTTP, and dATP, 10 uCi [³²P]-alpha-dGTP (800 Ci/mmol), 20 ug activated calf thymus DNA and the test compound at indicated concentrations. The alpha DNA polymerase reaction mixture was as follows in a 50 uL volume per sample: 20 mM Tris-HCl (pH 8), 5 mM magnesium acetate, 0.3 mg/mL BSA, 1 mM DTT, 0.1 mM spermine, 0.05 mM of dCTP, dTTP, and dATP, 10 uCi [³²P]-alpha-dGTP (800 Ci/mmol), 20 ug activated calf thymus DNA and the test compound at the indicated concentrations. For each assay, the enzyme reactions were allowed to proceed for 30 minutes at 37° C. followed by the transfer onto glass-fiber filter plates and subsequent precipitation with 10% trichloroacetic acid (TCA). The plate was then washed with 5% TCA followed by one wash with 95% ethanol. Once the filter had dried, incorporation of radioactivity was measured using a liquid scintillation counter (Microbeta).

### Example 58

### HIV Infected PBMC Assay:

Fresh human peripheral blood mononuclear cells (PBMCs) were obtained from a commercial source (Biological Specialty) and were determined to be seronegative for HIV and HBV. Depending on the volume of donor blood received, the leukophoresed blood cells were washed several times with PBS. After washing, the leukophoresed blood was diluted 1:1 with Dulbecco's phosphate buffered saline (PBS) and layered over 15 mL of Ficoll-Hypaque density gradient in a 50 ml conical centrifuge tube. These tubes were centrifuged for 30 min at 600 g. Banded PBMCs were gently aspirated from the resulting interface and washed three times with PBS. After the final wash, cell number was determined by Trypan Blue dye exclusion and cells were re-suspended at 1×10≢cells/mL in RPMI 1640 with 15% Fetal Bovine Serum (FBS), 2 mmol/L L-glutamine, 2 μg/mL PHA-P, 100 U/mL penicillin and 100 μg/mL streptomycin and allowed to incubate for 48-72 hours at 37° C. After incubation, PBMCs were centrifuged and resuspended in tissue culture medium. The cultures were maintained until use by half-volume culture changes with fresh IL-2 containing tissue culture medium every 3 days. Assays were initiated with PBMCs at 72 hours post PHA-P stimulation.

To minimize effects due to donor variability, PBMCs employed in the assay were a mixture of cells derived from 3 donors. Immediately prior to use, target cells were resuspended in fresh tissue culture medium at 1×10\hat{ }6 cells/mL and plated in the interior wells of a 96-well round bottom microtiter plate at 50 uL/well. Then, 100 μL of 2× concentrations of compound-containing medium was transferred to the 96-well plate containing cells in 50 μL of the medium. AZT was employed as an internal assay standard.

Following addition of test compound to the wells, 50 μL of a predetermined dilution of HIV virus (prepared from 4× of final desired in-well concentration) was added, and mixed well. For infection, 50-150 TCID₅₀ of each virus was added per well (final MOI approximately 0.002). PBMCs were exposed in triplicate to virus and cultured in the presence or absence of the test material at varying concentrations as described above in the 96-well microtiter plates. After 7 days in culture, HIV-1 replication was quantified in the tissue culture supernatant by measurement of reverse transcriptase (RT) activity. Wells with cells and virus only served as virus controls. Separate plates were identically prepared without virus for drug cytotoxicity studies.

Reverse Transcriptase Activity Assay—Reverse transcriptase activity was measured in cell-free supernatants using a standard radioactive incorporation polymerization assay. Tritiated thymidine triphosphate (TTP; New England Nuclear) was purchased at 1 Ci/mL and 1 uL was used per enzyme reaction. A rAdT stock solution was prepared by mixing 0.5 mg/mL poly rAand 1.7 U/mL oligo dT in distilled water and was stored at −20° C. The RT reaction buffer was prepared fresh daily and consists of 125 μL of 1 mol/L EGTA, 125 μL of dH₂O, 125 μL of 20% Triton X-100, 50 μL of 1 mol/L Tris (pH 7.4), 50 μL of 1 mol/L DTT, and 40 μL of 1 mol/L MgCl₂. For each reaction, 1 μL of TTP, 4 μL of dH₂O, 2.5 uL of rAdT, and 2.5 uL of reaction buffer were mixed. Ten microliters of this reaction mixture was placed in a round bottom microtiter plate and 15 μL of virus-containing supernatant was added and mixed. The plate was incubated at 37° C. in a humidified incubator for 90 minutes. Following incubation, 10 μL of the reaction volume was spotted onto a DEAE filter mat in the appropriate plate format, washed 5 times (5 minutes each) in a 5% sodium phosphate buffer, 2 times (1 minute each) in distilled water, 2 times (1 minute each) in 70% ethanol, and then air dried. The dried filtermat was placed in a plastic sleeve and 4 mL of Opti-Fluor O was added to the sleeve. Incorporated radioactivity was quantified utilizing a Wallac 1450 Microbeta Trilux liquid scintillation counter.

### Example 59

### HBV:

HepG2.2.15 cells (100 μL) in RPMI1640 medium with 10% fetal bovine serum was added to all wells of a 96-well plate at a density of 1×10⁴ cells per well and the plate was incubated at 37° C. in an environment of 5% CO₂ for 24 hours. Following incubation, six ten-fold serial dilutions of test compound prepared in RPMI1640 medium with 10% fetal bovine serum were added to individual wells of the plate in triplicate. Six wells in the plate received medium alone as a virus only control. The plate was incubated for 6 days at 37° C. in an environment of 5% CO₂. The culture medium was changed on day 3 with medium containing the indicated concentration of each compound. One hundred microliters of supernatant was collected from each well for analysis of viral DNA by qPCR and cytotoxicity was evaluated by XTT staining of the cell culture monolayer on the sixth day.

Ten microliters of cell culture supernatant collected on the sixth day was diluted in qPCR dilution buffer (40 μg/mL sheared salmon sperm DNA) and boiled for 15 minutes. Quantitative real time PCR was performed in 386 well plates using an Applied Biosystems 7900HT Sequence Detection System and the supporting SDS 2.4 software. Five microliters (5 μL) of boiled DNA for each sample and serial 10-fold dilutions of a quantitative DNA standard were subjected to real time Q-PCR using Platinum Quantitative PCR SuperMix-UDG (Invitrogen) and specific DNA oligonucleotide primers (IDT, Coralville, ID) HBV-AD38-qF1 (5′-CCG TCT GTG CCT TCT CAT CTG-3′), HBV-AD38-qRl (5′-AGT CCA AGA GTY CTC TTA TRY AAG ACC TT-3′), and HBV-AD38-qPl (5′-FAM CCG TGT GCA/ZEN/CTT CGC TTC ACC TCT GC-3′BHQ1) at a final concentration of 0.2 μM for each primer in a total reaction volume of 15 μL. The HBV DNA copy number in each sample was interpolated from the standard curve by the SDS.24 software and the data were imported into an Excel spreadsheet for analysis.

The 50% cytotoxic concentration for the test materials are derived by measuring the reduction of the tetrazolium dye XTT in the treated tissue culture plates. XTT is metabolized by the mitochondrial enzyme NADPH oxidase to a soluble formazan product in metabolically active cells. XTT solution was prepared daily as a stock of 1 mg/mL in PBS. Phenazine methosulfate (PMS) stock solution was prepared at 0.15 mg/mL in PBS and stored in the dark at −20° C. XTT/PMS solution was prepared immediately before use by adding 40 μL of PMS per 1 mL of XTT solution. Fifty microliters of XTT/PMS was added to each well of the plate and the plate incubated for 2-4 hours at 37° C. The 2-4 hour incubation has been empirically determined to be within linear response range for XTT dye reduction with the indicated numbers of cells for each assay. Adhesive plate sealers were used in place of the lids, the sealed plate was inverted several times to mix the soluble formazan product and the plate was read at 450 nm (650 nm reference wavelength) with a Molecular Devices SpectraMax Plus 384 spectrophotometer. Data were collected by Softmax 4.6 software and imported into an Excel spreadsheet for analysis.

### Example 60

### Dengue RNA-Dependent RNA Polymerase Reaction Conditions

RNA polymerase assay was performed at 30° C. using 100 μl reaction mix in 1.5 ml tube. Final reaction conditions were 50 mM Hepes (pH 7.0), 2 mM DTT, 1 mM MnCl₂, 10 mM KCl, 100 nM UTR-Poly A (self-annealing primer), 10 μM UTP, 26 nM RdRp enzyme. The reaction mix with different compounds (inhibitors) was incubated at 30° C. for 1 hour. To assess amount of pyrophosphate generated during polymerase reaction, 30 μl of polymerase reaction mix was mixed with a luciferase coupled-enzyme reaction mix (70 μl). Final reaction conditions of luciferase reaction were 5 mM MgCl₂, 50 mM Tris-HCl (pH 7.5), 150 mM NaCl, 200 μU ATP sulfurylase, 5 μM APS, 10 nM Luciferase, 100 μM D-luciferin. White plates containing the reaction samples (100 μl) were immediately transferred to the luminometer Veritas (Turner Biosystems, CA) for detection of the light signal.

### Example 61

### Procedure for Cell Incubation and Analysis

Huh-7 cells were seeded at 0.5×10\hat{ }6 cells/well in 1 mL of complete media in 12 well tissue culture treated plates. The cells were allowed to adhere overnight at 37°/5% CO₂. A 40 μM stock solution of test article was prepared in 100% DMSO. From the 40 μM stock solution, a 20 μM solution of test article in 25 ml of complete DMEM media was prepared. For compound treatment, the media was aspirated from the wells and 1 mL of the 20 μM solution was added in complete DMEM media to the appropriate wells. A separate plate of cells with “no” addition of the compound was also prepared. The plates were incubated at 37°/5% CO₂ for the following time points: 1, 3, 6 and 24 hours. After incubation at the desired time points, the cells were washed 2× with 1 mL of DPBS. The cells were extracted by adding 500 μl of 70% methanol/30% water spiked with the internal standard to each well treated with test article. The non-treated blank plate was extracted with 500 ul of 70% methanol/30% water per well. Samples were centrifuged at 16,000 rpm for 10 minutes at 4° C. Samples were analyzed by LC-MS/MS using an ABSCIEX 5500 QTRAP LC-MS/MS system with a Hypercarb (PGC) column.

### Example 62

### Zika RNA-Dependent RNA Polymerase Reaction Conditions

RNA polymerase assay was performed at 30° C. using 100 μl reaction mix in 1.5 ml tube. Final reaction conditions were 50 mM Hepes (pH 7.0), 2 mM DTT, 1 mM MnCl₂, 10 mM KCl, 100 nM UTR-Poly A (self-annealing primer), 10 μM UTP, 26 nM RdRp enzyme. The reaction mix with different compounds (inhibitors) was incubated at 30° C. for 1 hour. To assess amount of pyrophosphate generated during polymerase reaction, 30p1 of polymerase reaction mix was mixed with a luciferase coupled-enzyme reaction mix (70 μl).

Final reaction conditions of luciferase reaction were 5 mM MgCl₂, 50 mM Tris-HCl (pH 7.5), 150 mM NaCl, 200 μU ATP sulfurylase, 5 μM APS, 10 nM Luciferase, 100 μM D-luciferin. White plates containing the reaction samples (100 μl) were immediately transferred to the luminometer Veritas (Turner Biosystems, CA) for detection of the light signal.

### Example 63

### Zika Infectious Assay Conditions

Vero cells were passaged in DMEM medium in T-75 flasks prior to use in the antiviral assay. On the day preceding the assay, the cells were split 1:2 to assure they were in exponential growth phase at the time of infection. The cells were resuspended at 5×10³ cells per well in tissue culture medium and added to flat bottom microtiter plates in a volume of 100 mL. The plates were incubated at 37° C./5% CO₂ overnight to allow for cell adherence. Separately, Zika virus was titrated in LLCMK2 cells to define the inoculum for use in the antiviral assay. Virus was diluted in DMEM medium such that the amount of virus added to each well in a volume of 100 mL was the amount determined to achieve 85 to 95% cell killing at 5 days post-infection. Following incubation test plates were stained with XTT dye. XTT solution was prepared daily as a stock solution of 1 mg/mL in RPMI1640. PMS solution was prepared at 0.15 mg/mL in PBS and stored in the dark at −20° C. XTT/PMS stock was prepared immediately before use by adding 40 mL of PMS per mL of XTT solution. Fifty microliters of XTT/PMS was added to each well of the plate, and the plate was reincubated for 4 hours at 37° C. Plates were sealed with adhesive plate sealers ad shaken gently to mix the soluble formazan product, and the plate was read spectrophotometrically read 450/650 nm with a Molecular Devices Vmax plate reader. The raw data was collected from Softmax Pro and imported into a Microsoft Excel XLfit4 spreadsheet for analysis using four parameter curve fit calculations.

### Example 64

### POLRMT Methods.

### POLRMT Enzyme Purification

A variant of human POLRMT coding sequence was amplified from a POLRMT cDNA plasmid (Accession: BC098387, Clone ID: 5264127, Dharmacon, CO) and cloned into a pMal-c5× vector under control of the tac promoter. For protein expression, the plasmid was transformed into Stellar competent cells (Clontech). Expression vector pMal-c5× contains a lacI gene which allows inducible expression of POLRMT in Stellar cells. The transformed cells were grown in LB medium containing 100 μg/ml ampicillin at 35° C. to an optical density of 1 at 600 nm. Cells were cooled down in a 4° C. fridge for 1 hour. MgCl₂ was added to final concentration of 1 mM. Protein expression was induced at 16° C. overnight by the addition of 0.4 mM IPTG. Cells were harvested by centrifugation at 4000×g for 20 min at 4° C. The cell pellet was stored at −80° C. until further processed. For protein purification, the cell pellet was re-suspended in sonication buffer (20 mM Tris-HCl pH 7.5, 10% glycerol, 500 mM NaCl, 0.5% Triton X-100, 10 mM DTT, 10 mM MgCl₂, 30 mM imidazole and 1× protease inhibitor cocktail). Cell disruption was performed on ice for 10 min using an ultrasound probe sonicator. The cell extract was clarified by centrifugation at 16,000×g for 20 min at 4° C. The supernatant was incubated with HisPur Ni-NTA agarose resin with gentle rocking for 15 minutes at 4° C. The resin was then washed 5 times with 10 volumes of wash buffer (20 mM Tris-HCl pH 7.5, 10% glycerol, 500 mM NaCl, 0.1% Triton X-100, 1 mM DTT, 2 mM MgCl₂) containing 30 mM imidazole and then once with the wash buffer containing 2M NaCl. The protein was eluted from the resin with 1 volume of elution buffer (20 mM Tris-HCl, pH 7.5, 10% glycerol, 50 mM NaCl, 0.5% Triton X-100, 10 mM DTT and 300 mM imidazole). The eluted enzyme was adjusted to 50% glycerol and stored at −80° C. before use. Protein identification was performed by mass spectrometry. The concentration of a targeted protein was measured by SDS-PAGE using BSA (Sigma, St. Louis, MO) as a standard.

### Measurement of Ribonucleotide Analog Incorporation Efficiency

Different templates were designed to test individual analog rNTPs, Table 1. Different concentrations of tested ribonucleotide analogs were added to reaction mixtures containing 10 nM P/T and 20 nM POLRMT in a reaction buffer (5 mM Tris-HCl, pH 7.5, 10 mM DTT, 20 mM MgCl₂, 0.5% X-100, 10% glycerol) to initiate the reactions. The reactions were continued at 22° C. for different time and subsequently quenched with quenching buffer (8 M Urea, 90 mM Tris base, 29 mM taurine, 10 mM EDTA, 0.02% SDS and 0.1% bromophenol blue). The quenched samples were denatured at 95° C. for 15 min and the primer extension products were separated using 20% denaturing polyacrylamide gel electrophoresis (Urea PAGE) in 1×TTE buffer (90 mM Tris base, 29 mM Taurine and 0.5 mM EDTA). After electrophoresis, gels were scanned using an Odyssey infrared imaging system. The intensity of different RNA bands was quantified using Image Studio Software Lite version 4.0. The incorporation efficiencies of different rNTP analogs were evaluated by measurement the K_{1/2} and corresponding Discrimination Values (ref. G Lu).

### Primer Extension Polymerase Activity Assay

POLRMTs polymerase activity was determined in a primer extension reaction using a fluorescently labeled RNA primer/DNA template complex. A typical primer extension reaction was performed in a 20-μl reaction mixture containing reaction buffer (5 mM Tris-HCl, pH7.5, 10 mM DTT, 20 mM MgCl₂, 0.1% Triton X-100, 0.01 U RNasin, 10% glycerol), 10 nM P/T complex, and 20 nM POLRMT. The reaction was initiated by the addition of rNTPs at a final concentration of 100 μM, followed by incubation for 1 h at 22° C. The reactions were quenched by the addition of 20 μl quenching buffer (8 M Urea, 90 mM Tris base, 29 mM taurine, 10 mM EDTA, 0.02% SDS and 0.1% bromophenol blue).

The quenched samples were denatured at 95° C. for 15 min and the primer extension products were separated using 20% denaturing polyacrylamide gel electrophoresis (Urea PAGE) in 1×TTE buffer (90 mM Tris base, 29 mM Taurine and 0.5 mM EDTA). After electrophoresis, gels were scanned using an Odyssey infrared imaging system (LI-COR Biosciences, Lincoln, NE). The images were analyzed and the proper RNA bands were quantified using Image Studio software Lite version 4.0 (LI-COR Biosciences, Lincoln, NE).

### Example 65

EIDD-02838 Togaviridae Activity

| | Cell | EC₅₀ | EC₉₀ | CC₅₀ |
|---|---|---|---|---|
| Virus | Line | (μM) | (μM) | (μM) |
| VEEV | Vero76 | 8.28 | | >230 |
| VEEV | Vero76 | 8.51 | | >230 |
| EEEV | Vero76 | 2.76 | | >230 |
| EEEV | Vero76 | 3.22 | | >230 |
| EEEV | Vero 76 | | 28.4 | >230 |
| EEEV | Vero 76 | 12.4 | | >230 |
| WEEV | Vero76 | 5.98 | | >230 |
| WEEV | Vero76 | 7.82 | | >230 |
| WEEV | Vero 76 | | 24.15 | >230 |
| WEEV | Vero 76 | 5.75 | | >230 |
| CHIKV | Vero76 | 2.3 | | >230 |
| CHIKV | Vero76 | 3.68 | | >230 |
| CHIKV | Vero 76 | | 23.92 | >230 |
| CHIKV | Vero 76 | 3.91 | | >230 |

### Example 66

EIDD-02838 Bunyaviridae Activity

| | Cell | EC₅₀ | EC₉₀ | CC₅₀ |
|---|---|---|---|---|
| Virus | Line | (μM) | (μM) | (μM) |
| RVFV | Vero76 | 3.91 | | >230 |
| RVFV | Vero76 | 3.68 | | >230 |
| RVFV | Vero 76 | | 2.19 | >230 |
| RVFV | Vero 76 | 4.14 | | >230 |
| Heartland virus | Vero | | 8.05 | >230 |
| La Crosse virus | Vero 76 | <0.23 | | >230 |
| La Crosse virus | Vero 76 | <0.23 | | 193 |
| La Crosse virus | Vero 76 | | 0.12 | >23 |
| La Crosse virus | Vero 76 | 0.69 | | >23 |
| Marpol virus | Vero | 25.3 | | >230 |
| Marpol virus | Vero | 19.1 | | >230 |
| Punta Toro virus | Vero 76 | 6.21 | | >230 |
| Punta Toro virus | Vero 76 | 5.75 | | >230 |
| Severe fever | Vero | | 7.82 | >230 |
| thrombocytopenia | | | | |
| syndrome virus | | | | |

### Example 67

EIDD-02838 Arenaviridae Activity

| | Cell | EC₅₀ | EC₉₀ | CC₅₀ |
|---|---|---|---|---|
| Virus | Line | (μM) | (μM) | (μM) |
| Lassa fever virus | Vero | | 0.14 | >230 |
| Lassa fever virus | Vero | | 1.3 | 213 |
| Junin virus | Vero | 0.37 | | >230 |
| Junin virus | Vero | <0.23 | | >230 |
| Junin virus | Vero | | 0.023 | >23 |
| Junin virus | Vero | 0.023 | | >23 |
| Lymphocytic | Vero | | 4.6 | >230 |
| choriomeningitis virus | | | | |
| Pichinde virus | Vero | 2.76 | | >230 |
| Pichinde virus | Vero | 1.45 | | >230 |
| Pichinde virus | Vero | | 0.55 | >23 |
| Pichinde virus | Vero | 0.35 | | >23 |
| Tacaribe virus | Vero | <0.23 | | >230 |
| Tacaribe virus | Vero | <0.23 | | >230 |
| Tacaribe virus | Vero | | 0.046 | >23 |
| Tacaribe virus | Vero | 0.046 | | >23 |

### Example 68

EIDD-02838 Influenza Activity

| | | | | |
|---|---|---|---|---|
| | Cell | EC₅₀ | EC₉₀ | CC₅₀ |
| Virus | Line | (μM) | (μM) | (μM) |

| | | | |
|---|---|---|---|
| Influenza A H1N1 | MDCK | 1.2 | >230 |
| Influenza A H1N1 | MDCK | 1.59 | >230 |
| Flu A H7N9 (High Path) | MDCK | 5.29 | >230 |
| Flu A H7N9 (High Path) | MDCK | 4.37 | >230 |
| Flu A H5N1 (High Path) | MDCK | 23 | >230 |
| Flu A H5N1 (High Path) | MDCK | 11.27 | >230 |
| Flu A H1N1 | MDCK | 1.98 | >230 |
| Flu A H1N1 | MDCK | 2.3 | >230 |
| Flu A H3N2 | MDCK | 0.97 | >230 |
| Flu A H3N2 | MDCK | 0.87 | >230 |
| Flu A H5N1 (Low Path) | MDCK | 0.78 | >230 |
| Flu A H5N1 (Low Path) | MDCK | 0.71 | >230 |
| Flu B | MDCK | 0.78 | >230 |
| Flu B | MDCK | 0.74 | >230 |
| Flu B | MDCK | 0.74 | >230 |
| Flu B | MDCK | 0.67 | >230 |

### Example 69

EIDD-02838 Parainfluenza and RSV Activity

| | | | | |
|---|---|---|---|---|
| | Cell | EC₅₀ | EC₉₀ | CC₅₀ |
| Virus | Line | (μM) | (μM) | (μM) |

| | | | |
|---|---|---|---|
| Parainfluenza virus 3 | MA-104 | 73.6 | >230 |
| Parainfluenza virus 3 | MA-104 | 78.2 | >230 |
| RSV | MA-104 | 3.45 | >230 |
| RSV | MA-104 | 2.76 | >230 |
| RSV | MA-104 | 7.82 | >230 |
| RSV | MA-104 | 6.21 | >230 |

### Example 70

EIDD-02838 Ebola Activity

| | Cell | EC₅₀ | EC₉₀ | CC₅₀ |
|---|---|---|---|---|
| Virus | Line | (μM) | (μM) | (μM) |
| EBOV | Vero | 149.5 | | >230 |

### Example 71

EIDD-02838 Coronaviridae Activity

| | Cell | EC₅₀ | EC₉₀ | CC₅₀ |
|---|---|---|---|---|
| Virus | Line | (μM) | (μM) | (μM) |
| MERS | Vero76 | 1.98 | | >230 |
| MERS | Vero E6 | 2.53 | | >230 |

### Example 72

EIDD-02838 Flaviviridae Activity

| | Cell | EC₅₀ | EC₉₀ | CC₅₀ |
|---|---|---|---|---|
| Virus | Line | (μM) | (μM) | (μM) |
| POWV | BHK | 75.9 | | >230 |
| POWV | BHK | 98.9 | | >230 |
| ZIKV | Vero 76 | 7.82 | | >230 |
| ZIKV | Vero 76 | 9.2 | | >230 |
| DENV2 | Vero 76 | | 29.9 | >230 |
| DENV2 | Vero 76 | >230 | | >230 |
| JEV | Vero 76 | | 3.91 | >230 |
| JEV | Vero 76 | 78.2 | | >230 |
| Usutu virus | Vero 76 | | 25.76 | >230 |
| Usutu virus | Vero 76 | 6.9 | | >230 |
| ZIKV | Vero 76 | | 43.7 | 156.4 |
| ZIKV | Vero 76 | 8.28 | | 156.4 |

### Example 73

EIDD-02838 Picornaviridae Activity

| | Cell | EC₅₀ | EC₉₀ | CC₅₀ |
|---|---|---|---|---|
| Virus | Line | (μM) | (μM) | (μM) |
| Poliovirus | Vero 76 | 7.36 | | >230 |
| Poliovirus | Vero 76 | 8.74 | | >230 |
| Coxsackie virus B3 | Vero 76 | 4.83 | | >230 |
| Coxsackie virus B3 | Vero 76 | 2.76 | | >230 |
| Enterovirus-68 | RD | 7.36 | | >230 |
| Enterovirus-68 | RD | 7.82 | | >230 |
| HRV-14 | HeLa-Ohio | >230 | | >230 |
| HRV-14 | HeLa-Ohio | >230 | | >230 |

### Example 74

EIDD-02749 Norovirus Activity

| | Cell | EC₅₀ | EC₉₀ | CC₅₀ |
|---|---|---|---|---|
| Virus | Line | (μM) | (μM) | (μM) |
| Murine Norovirus | RAW264.7 | 2.2 | 7.2 | >100 |

### Example 75

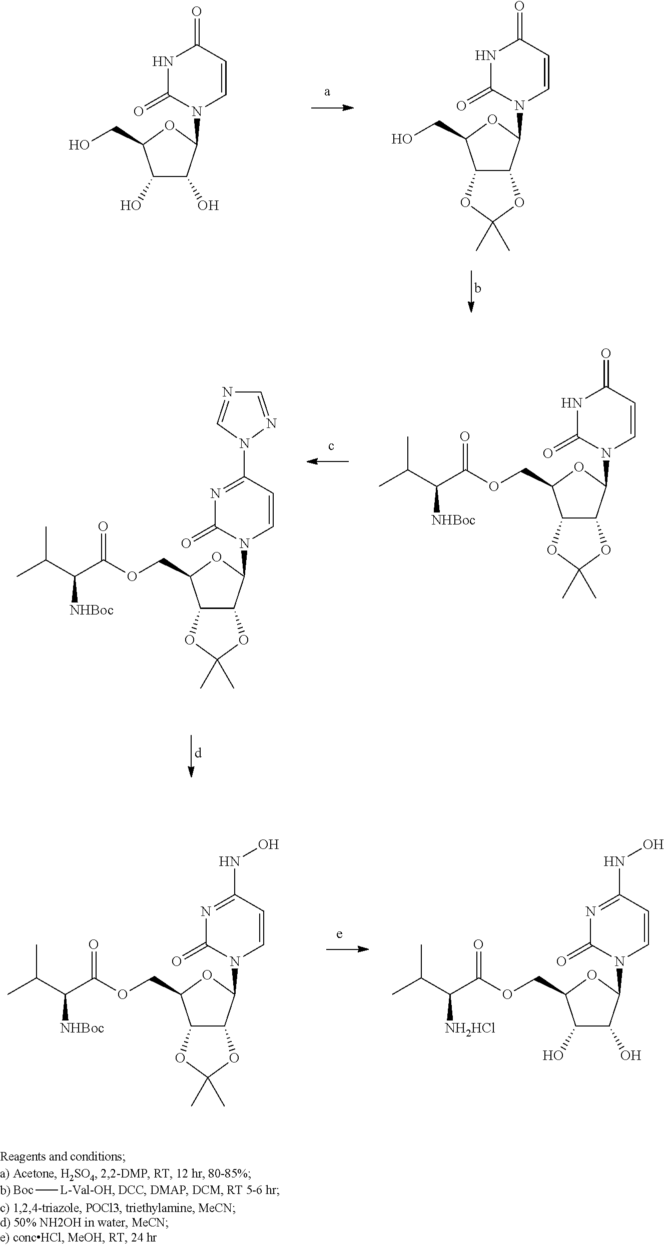

A 2 L 3-neck RBF was charged with 1-[(3R, 4S, 5R)-3,4-dihydroxy-5-(hydroxymethyl)tetrahydrofuran-2-yl]pyrimidine-2,4-dione (61.4 g, 251.43 mmol) and acetone (1400 mL). The resulting slurry was stirred at RT and sulfuric acid (2 mL was added. Stirring was continued overnight. The clear colorless solution was quenched/adjusted to basic pH with 100 mL of trimethylamine. The crude solution was concentrated under reduced pressure to yield a pale yellow oil. The residue was dissolved in 600 mL of EtOAc and washed with water×2, bicarb×2, water, brine×2 and dried over sodium sulfate. The colorless solution was concentrated under reduced pressure to yield 1-[(3aR, 6R, 6aR)-6-(hydroxymethyl)-2,2-dimethyl-3a, 4,6,6a-tetrahydrofuro[3,4-d][1,3]dioxol-4-yl]pyrimidine-2,4-dione (45 g) as a white solid.

A 200 mL RBF was charged with 1-[(3aR, 6R, 6aR)-6-(hydroxymethyl)-2,2-dimethyl-3a, 4,6,6a-tetrahydrofuro[3,4-d][1,3]dioxol-4-yl]pyrimidine-2,4-dione (2.36 g, 8.3 mmol) and DCM (50 mL). The reaction was stirred until a solution was formed. Next, (2S)-2-(tert-butoxycarbonylamino)-3-methyl-butanoic acid (2.16 g, 9.96 mmol) and N,N-dimethylpyridin-4-amine (0.1 g, 0.8300 mmol) were added. The reaction was cooled to 0° C. with an ice bath. A DCM solution of N,N′-dicyclohexylcarbodiimide (2.06 g, 9.96 mmol) was added slowly. The reaction mixture was allowed to warm to rt. Monitored by TLC (EtOAc).

A precipitate (DCU) formed after about 1 hr and no starting material was detected after 3 hrs. The solids were filtered off and rinsed with EtOAc. The filtrate was washed with water, brine, dried over sodium sulfate and concentrated under reduced pressure to yield white, gooey solid. The gummy solid was triturated with ether and filtered to remove the solid. The filtrate was concentrated under reduced pressure to yield about 8 g of thick viscous oil. The product was purified by SGC, pooled fractions 6-25 and concentrated under reduced pressure to yield [(3aR, 6R, 6aR)-4-(2,4-dioxopyrimidin-1-yl)-2,2-dimethyl-3a, 4,6,6a-tetrahydrofuro[3,4-d][1,3]dioxol-6-yl]methyl (2S)-2-(tert-butoxycarbonylamino)-3-methyl-butanoate (3.8 g, 7.8592 mmol, 94.667% yield) as a foamy white solid after drying in vacuo. 1,2,4-triazole was taken in anhydrous acetonitrile and stirred at RT after 30 min, the reaction mixture was cooled to 0° C. and POCl₃ was added dropwise and continued stirring for 2 hr. After 2 hr triethylamine was added dropwise and continue stirring for 1 hr, the reaction mixture was slowly brought to RT, and the uridine derived substrate from the above reaction was added as solution in acetonitrile. The reaction mixture stirred at RT overnight. After completion of the reaction, the solvent was removed under reduced pressure and taken in DCM and extracted with water. The organic layer was dried over anhydrous sodium sulphate and concentrated under reduced pressure. The crude product was purified by flash column chromatography.

To a solution of the substrate in acetonitrile (10 mL/gm), 50% hydroxylamine in water was added dropwise and stirred at rt for 2-3 hrs. After completion of the reaction, solvent was removed under reduced pressure and the crude product was purified by flash column chromatography using hexane and EtOAc as eluent.

1 g of substrate was taken in 20 mL of methanol and treated with 2 mL of conc.HCl (36%) and after 3-4 hr 30% completion was observed. Another 5 mL of conc.HCl was added and stirred overnight. After completion of the reaction, solvent was removed and the crude product was taken in minimum methanol and added dropwise to excess diethylether with stirring, product was crashed out of solution and allowed to settle, ether was decanted and fresh ether was added, stirred, settled and decanted, the same process was repeated two times. After ether was decanted, solid was dried over a rotavap and high vacuum to get free flowing white solid. Ether was trapped in the solid and was difficult to remove. The solid was dissolved in methanol, evaporated and dried to get colorless foam, which still holds methanol. The foam was taken in water and a purple solution was observed. The purple solution was purified by reverse phase ISCO column chromatography using water and acetonitrile. The fractions containing product were evaporated under reduced pressure and lyophilized to get colorless solid.

### Example 76

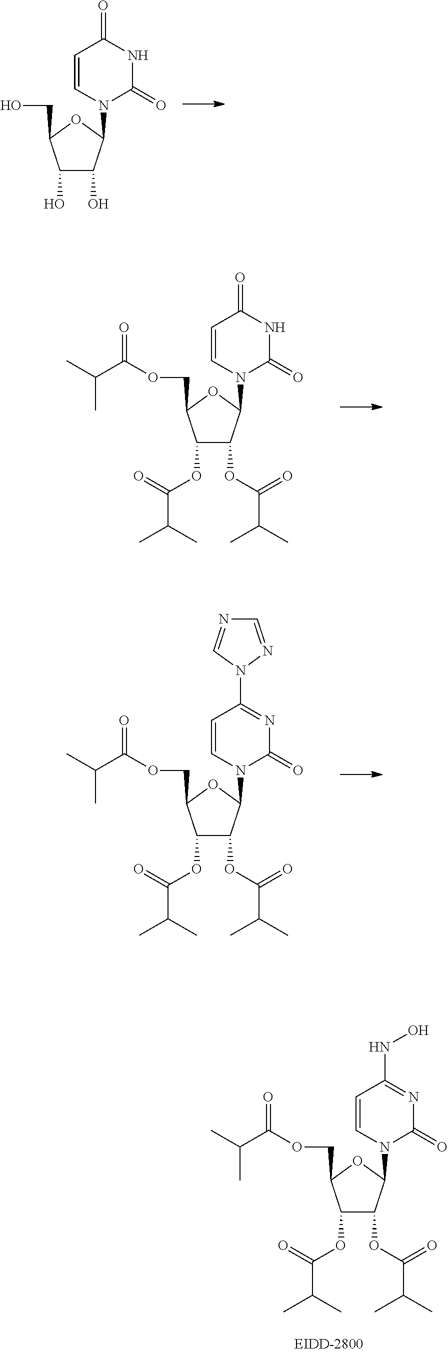 [SMILES: O=c1ccn([C@@H]2O[C@H](CO)[C@@H](O)[C@H]2O)c(=O)[nH]1 ; CC(C)C(=O)OC[C@H]1O[C@@H](n2ccc(=O)[nH]c2=O)[C@H](OC(=O)C(C)C)[C@@H]1OC(=O)C(C)C ; CC(C)C(=O)OC[C@H]1O[C@@H](n2ccc(-n3cncn3)nc2=O)[C@H](OC(=O)C(C)C)[C@@H]1OC(=O)C(C)C ; CC(C)C(=O)OC[C@H]1O[C@@H](n2ccc(NO)nc2=O)[C@H](OC(=O)C(C)C)[C@@H]1OC(=O)C(C)C]

A 3-neck 1 L round bottom flask equipped with an overhead stirrer, temperature probe and addition funnel was charged with uridine (25 g, 102.38 mmol) and ethyl acetate (500 mL). The white slurry was stirred at ambient temperature while triethylamine (71.39 mL, 511.88 mmol) and DMAP (0.63 g, 5.12 mmol) were added to the mixture. The slurry was cooled in an ice bath and isobutyric anhydride (56.02 mL, 337.84 mmol) was slowly added to the reaction mixture over a 5 minute period. The temperature rose 25° C. during the addition. The resulting slurry was stirred at ambient temperature and monitored by TLC. After 1 hour, a clear colorless solution had formed and TLC showed no starting material. The reaction was quenched with 200 mL of water, stirred at rt for 20 minutes. The layers were separated, and the organics were washed with water (2×100 mL), saturated aqueous bicarbonate solution (100 mL×2), 100 mL of water, brine (100 mL×2), and then dried over sodium sulfate. The organics were filtered and the filtrate was concentrated under reduced pressure at 45° C. to yield a yellow oil. The oil was used in the next step without any further purification.

A 2 L 3-neck flask equipped with an argon inlet, overhead stirrer and temperature probe was charged with 1H-1,2,4-triazole (50.88 g, 736.68 mmol), triethylamine (114.17 mL, 818.54 mmol) and MeCN (350 mL). The reaction mixture was stirred at rt for 20 minutes. An ethyl acetate (350 mL) solution of [(2R, 3R, 4R)-5-(2,4-dioxopyrimidin-1-yl)-3,4-bis(2-methylpropanoyloxy)tetrahydrofuran-2-yl]methyl 2-methylpropanoate (46.5 g, 102.32 mmol) was added and the mixture was cooled to <5° C. using an ice bath. Stirring continued for 20 minutes. Next, phosphorous(V)oxychloride (14.35 mL, 153.48 mmol) was added slowly under argon at less than 20° C. over 15 minutes. The reaction was monitored by TLC (100% EtOAc), starting material (R_{f}=0.89) consumed in less than 2 hours and a new spot due to product (R_{f}=0.78) present. The reaction was quenched with 500 mL of water and 400 mL of EtOAc. The quenched reaction was allowed to stir at rt for 15 minutes. The layers were separated and the organic layer was washed with water (2×100 mL), 200 mL of 0.5N HCl, and brine (2×100 mL). The organics were dried over sodium sulfate, filtered and concentrated under reduced pressure to yield [(2R, 3R, 4R)-3,4-bis(2-methylpropanoyloxy)-5-[2-oxo-4-(1,2,4-triazol-1-yl)pyrimidin-1-yl]tetrahydrofuran-2-yl]methyl 2-methylpropanoate (49 g, 96.93 mmol, 94.735% yield) as a yellow oil. The crude material was used in the next step without further purification.

A 500 mL round bottom flask was charged with [(2R, 3R, 4R)-3,4-bis(2-methylpropanoyloxy)-5-[2-oxo-4-(1,2,4-triazol-1-yl)pyrimidin-1-yl]tetrahydrofuran-2-yl]methyl 2-methylpropanoate (48.9 g, 96.73 mmol), ethyl acetate (400 mL), and isopropyl alcohol (100 mL). The reaction mixture was stirred at rt until all of the starting material was dissolved. The orange solution was treated with hydroxylamine (6.52 mL, 106.41 mmol), and the resulting pale yellow solution was stirred at rt and monitored by TLC (EtOAc). No starting material was observed after 1 hour. The reaction was quenched with 500 mL of water, and the layers were separated. The organics were washed with 100 mL of water, 100 mL×2 of brine, and then dried over sodium sulfate. The organics were filtered and concentrated under reduced pressure to yield the crude product. The crude product was dissolved in 180 mL of hot MTBE and allowed to cool to rt. Seed crystals were added, and the flask was placed in the freezer. The white solid that formed was collected by filtration, washed with a minimal amount of MTBE and dried in vacuo to yield the desired product.

### Example 77

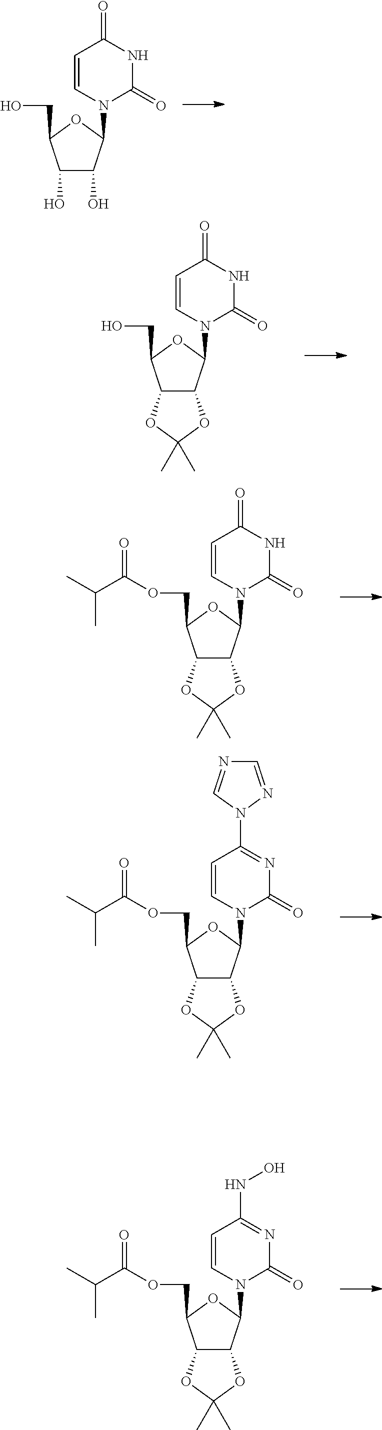

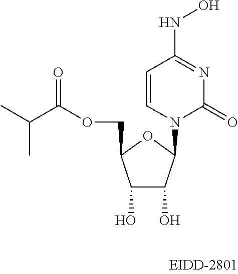 [SMILES: CC(C)C(=O)OC[C@H]1O[C@@H](n2ccc(NO)nc2=O)[C@H](O)[C@@H]1O]

A 1 L round bottom flask was charged with uridine (25 g, 102.38 mmol) and acetone (700 mL). The reaction mixture was allowed to stir at rt. The slurry was then treated with sulfuric acid (0.27 mL, 5.12 mmol). Stirring was allowed to continue at rt for 18 hours. The reaction was quenched with 100 mL of trimethylamine and was used in the next step without further purification.

A 1 L round bottom flask was charged with the reaction mixture from the previous reaction. Triethylamine (71.09 mL, 510.08 mmol) and 4-dimethylaminopyridine (0.62 g, 5.1 mmol) were then added. The flask was cooled using an ice bath and then 2-methylpropanoyl 2-methylpropanoate (17.75 g, 112.22 mmol) was slowly added. The reaction mixture was allowed to stir at rt until the reaction was complete. The reaction mixture was concentrated under reduced pressure, and the residue was dissolved in 600 mL ethyl acetate and washed with saturated aqueous bicarbonate solution×2, water×2 and brine×2. The organics were dried over sodium sulfate and concentrated under reduced pressure to yield a clear colorless oil. The crude product was used in the next step without further purification.

A 1 L round bottom flask was charged with the crude product from above (36 g, 101.59 mmol) and MeCN (406.37 mL). The reaction mixture was allowed to stir until all the starting material was dissolved. Next, 1,2,4-triazole (50.52 g, 731.46 mmol) was added followed by the addition of N,N-diethylethanamine (113.28 mL, 812.73 mmol). The reaction mixture was allowed to stir at rt until all solids dissolved. The reaction was then cooled to 0° C. using an ice bath. Phosphorous oxychloride (24.44 mL, 152.39 mmol) was added slowly. The slurry that formed was allowed to stir under argon while slowly warming to rt. The reaction was then allowed to stir until complete by TLC (EtOAc). The reaction was then quenched by the addition of 100 mL of water. The slurry then became a dark colored solution, which was then concentrated under reduced pressure. The residue was dissolved in DCM and washed with water and brine. The organics were then dried over sodium sulfate, filtered, and concentrated under reduced pressure. The product was purified by silica gel chromatography (2×330 g columns). All fractions containing product were collected and concentrated under reduced pressure.

A 500 mL round bottom flask was charged with the product from the previous step (11.8 g, 29.11 mmol) and isopropyl alcohol (150 mL). The reaction mixture was allowed to stir at rt until all solids dissolved. Next, hydroxylamine (1.34 mL, 43.66 mmol) was added and stirring continued at ambient temperature. When the reaction was complete (HPLC) some solvent was removed under high vacuum at ambient temperature. The remaining solvent was removed under reduced pressure at 45° C. The resulting residue was dissolved in EtOAc and was washed with water and brine. The organics were dried over sodium sulfate, filtered, and concentrated under reduced pressure to yield oil. Crystals formed upon standing at rt. The crystals were collected by filtration, washed with ether×3, and dried in vacuo to provide the product as a white solid.

A 200 mL round bottom flask was charged with the product from the previous step (6.5 g, 17.6 mmol) and formic acid (100 mL, 2085.6 mmol). The reaction mixture was allowed to stir at rt overnight. The progress of the reaction was monitored by HPLC. The reaction mixture was concentrated under reduced pressure at 42° C. to yield a clear, pale pink oil. Next, 30 mL of ethanol was added. Solvent was then removed under reduced pressure. MTBE (50 mL) was added to the solid and heated. Next, isopropyl alcohol was added and heating was continued until all solid material dissolved (5 mL). The solution was then allowed to cool and stand at rt. A solid started to form after about 1 hr. The solids were collected by filtration, washed with MTBE, and dried in vacuo to yield the EIDD-2801 as a white solid. The filtrate was concentrated under reduced pressure to yield a sticky solid, which was dissolved in a small amount of isopropyl alcohol with heating. The solution was allowed to stand at rt overnight. A solid formed in the flask, which was collected by filtration, rinsed with isopropyl alcohol and MTBE, and dried in vacuo to an additional crop of desired product.

EIDD-2801 (25 g) was dissolved in 250 mL of isopropyl alcohol by heating to 70° C. to give a clear solution. The warm solution was polish filtered and filtrate transferred to 2 L three neck flask with overhead stirrer. It was warmed back to 70° C. and MTBE (250 mL) was slowly added into the flask. The clear solution was seeded and allowed to cool slowly to rt with stirring for 18 hrs. The EIDD-2801 solid that formed was filtered and washed with MTBE and dried at 50° C. under vacuum for 18 hours. The filtrate was concentrated, redissolved in 50 mL isopropyl alcohol and 40 mL MTBE by warming to give clear solution and allowed to stand at rt to give a second crop of EIDD-2801.

### Example 78. Norovirus Activity for EIDD-02749

| | | Cell | EC₅₀ | EC₉₀ | CC₅₀ |
|---|---|---|---|---|---|
| Virus | Strain | Line | (μM) | (μM) | (μM) |
| Murine Norovirus | MNV-1 | RAW264.7 | 2.2 | 7.2 | >100 |
| Human Norovirus | G1 | HG23 | 2.7 | 9.3 | 100 |

### Example 79. Togaviridae Activity for EIDD-02749

| | | Cell | EC₅₀ | EC₉₀ | CC₅₀ |
|---|---|---|---|---|---|
| Virus | Strain | Line | (μM) | (μM) | (μM) |
| CHIKV | S27 (VR-64) | Vero76 | 1.98 | | >380 |
| CHIKV | S27 (VR-64) | Vero76 | 2.05 | | >380 |
| CHIKV | S27 (VR-64) | Vero 76 | | 9.12 | >380 |
| CHIKV | S27 (VR-64) | Vero 76 | 3.42 | | >380 |
| VEEV | TC-83 | Huh-7 | 1.6 | | >380 |
| VEEV | TC-83 | Huh-7 | 19 | | >380 |
| VEEV | TC-83 | Vero 76 | | 213 | >380 |
| VEEV | TC-83 | Vero 76 | 7.6 | | >380 |
| EEEV | FL93-939 | Vero76 | 380 | | >380 |
| EEEV | FL93-939 | Vero76 | >380 | | >380 |
| WEEV | California | Vero76 | 9.88 | | >380 |
| WEEV | California | Vero76 | 11.8 | | >380 |

### Example 80. Flaviviridae Activity for EIDD-02749

| | | Cell | EC₅₀ | EC₉₀ | CC₅₀ |
|---|---|---|---|---|---|
| Virus | Strain | Line | (μM) | (μM) | (μM) |
| DENV2 | New Guinea C | Huh-7 | >380 | | >380 |
| DENV2 | New Guinea C | Huh-7 | >380 | | >380 |
| ZIKV | MR766 | Vero 76 | 16.3 | | >380 |
| ZIKV | MR766 | Vero 76 | 3.8 | | >380 |
| ZIKV | MR766 | Vero 76 | | >380 | >380 |
| ZIKV | MR766 | Vero 76 | 28.1 | | >380 |

Example 81. Picornaviridae Activity for EIDD-02749

| | | Cell | EC₅₀ | EC₉₀ | CC₅₀ |
|---|---|---|---|---|---|
| Virus | Strain | Line | (μM) | (μM) | (μM) |
| ENTV71 | Tainan/4643/98 | Vero 76 | 3.27 | | >380 |
| ENTV71 | Tainan/4643/98 | Vero 76 | 1.33 | | >380 |
| Coxsackie virus B3 | HA201933 | Vero 76 | 3.12 | | >380 |
| Coxsackie virus B3 | HA201933 | Vero 76 | 3.15 | | >380 |
| Coxsackie virus B3 | HA201933 | Vero 76 | | >380 | >380 |
| Coxsackie virus B3 | HA201933 | Vero 76 | 12 | | >380 |
| Enterovirus-68 | US/KY/14-18953 | RD | 13.7 | | >380 |
| Enterovirus-68 | US/KY/14-18953 | RD | 11.8 | | >380 |
| Poliovirus | Mahoney | Vero 76 | 12.2 | | >380 |
| Poliovirus | Mahoney | Vero 76 | 11.8 | | >380 |

Example 82. Respiratory Virus Activity for EIDD-02749

| | | | EC₅₀ | CC₅₀ |
|---|---|---|---|---|
| Virus | Strain | Cell Line | (μM) | (μM) |
| Influenza A H1N1 | CA/07/20/09 | MDCK | <0.38 | >380 |
| Influenza A H1N1 | CA/07/20/09 | MDCK | <0.38 | >380 |
| Flu A H3N2 | Perth/16/2009 | MDCK | <0.38 | >380 |
| Flu A H3N2 | Perth/16/2009 | MDCK | <0.38 | >380 |
| Flu A H5N1 | Duck/MN/1525/81 | MDCK | <0.38 | >380 |
| (Low Path) | | | | |
| Flu A H5N1 | Duck/MN/1525/81 | MDCK | <0.38 | >380 |
| (Low Path) | | | | |
| Flu B | Brisbane/60/2008 | MDCK | <0.38 | >380 |
| Flu B | Brisbane/60/2008 | MDCK | <0.38 | >380 |
| Flu B | Florida/4/2006 | MDCK | <0.38 | >380 |
| Flu B | Florida/4/2006 | MDCK | <0.38 | >380 |
| Parainfluenza | 14702 | MA-104 | 98.8 | >380 |
| virus 3 | | | | |
| Parainfluenza | 14702 | MA-104 | 60.8 | >380 |
| virus 3 | | | | |
| HRV-14 | 1059 | HeLa-Ohio | 1.22 | 32.7 |
| HRV-14 | 1059 | HeLa-Ohio | 1.14 | 31.9 |
| RSV | A2 | MA-104 | 1.22 | >380 |
| RSV | A2 | MA-104 | <0.38 | >380 |

### Example 83. Coronavirus Activity for EIDD-02749

| Virus | Strain | Cell Line | EC₅₀ (μM) | CC₅₀ (μM) |
|---|---|---|---|---|
| MERS | EMC | Vero76 | >380 | >380 |
| MERS | EMC | Vero76 | >380 | >380 |

### Example 84. Bunyaviridae Activity for EIDD-02749

| | | | EC₅₀ | EC₉₀ | CC₅₀ |
|---|---|---|---|---|---|
| Virus | Strain | Cell Line | (μM) | (μM) | (μM) |
| RVFV | MP-12 | Vero76 | 3.46 | | >380 |
| RVFV | MP-12 | Vero76 | 2.96 | | >380 |
| RVFV | MP-12 | Vero 76 | | 23.9 | >380 |
| RVFV | MP-12 | Vero 76 | 16.3 | | >380 |
| RVFV | MP-12 | Vero 76 | | 4.3 | >380 |
| Heartland virus | MO-4 | Vero | | 121.6 | >380 |
| La Crosse virus | Wisconsin 1960 | Vero 76 | <0.38 | | >380 |
| | (VR-744) | | | | |
| La Crosse virus | Wisconsin 1960 | Vero 76 | <0.38 | | >380 |
| | (VR-744) | | | | |
| La Crosse virus | Wisconsin 1960 | Vero 76 | | 0.27 | >380 |
| | (VR-744) | | | | |
| La Crosse virus | Wisconsin 1960 | Vero 76 | 0.38 | | >380 |
| | (VR-744) | | | | |
| Marpol virus | HV97021050 | Vero E6 | 95 | | >380 |
| Marpol virus | HV97021050 | Vero E6 | 57 | | >380 |
| Punta Toro virus | Adames | Vero 76 | 72.2 | | >380 |
| Punta Toro virus | Adames | Vero 76 | 91.2 | | >380 |
| Severe fever | HB-29 | Vero | | >380 | >380 |
| thrombocytopenia | | | | | |
| syndrome virus | | | | | |

### Example 85. Arenaviridae Activity for EIDD-02749

| | | | EC₅₀ | EC₉₀ | CC₅₀ |
|---|---|---|---|---|---|
| Virus | Strain | Cell Line | (μM) | (μM) | (μM) |
| Tacaribe virus | TRVL 11573 | Vero | <0.38 | | >380 |
| Tacaribe virus | TRVL 11573 | Vero | <0.38 | | >380 |
| Tacaribe virus | TRVL 11573 | Vero | | 0.87 | >380 |
| Tacaribe virus | TRVL 11573 | Vero | 0.34 | | >380 |
| Lassa fever virus | Josiah | Vero | 0.38 | | >380 |
| Lassa fever virus | Josiah | Vero | | 9 | >380 |
| Lymphocytic | Armstrong | Vero | | 7.22 | >380 |
| choriomeningitis virus | | | | | |
| Pichinde virus | An 4763 | Vero | 2.81 | | >380 |
| Pichinde virus | An 4763 | Vero | 1.03 | | >380 |
| Pichinde virus | An 4763 | Vero | | 0.19 | >380 |
| Junin virus | Candid #1 | Vero | 1.86 | | >380 |
| Junin virus | Candid #1 | Vero | 1.29 | | >380 |
| Junin virus | Candid #1 | Vero | | 0.38 | >380 |

### Example 86. Filovirus Activity for EIDD-02749

| | | | EC₅₀ | EC₉₀ | CC₅₀ |
|---|---|---|---|---|---|
| Virus | Strain | Cell Line | (μM) | (μM) | (μM) |
| EBOV | Zaire | Vero | 1.52 | | >380 |
| EBOV | Zaire | Vero | | 22 | >380 |
| Marburg virus | Angola | Vero | >380 | | >380 |

### Example 80. EIDD-02749 Cytotoxicity

| ID | Cell Line | CC50 |
|---|---|---|
| EIDD-02749 | CEM | 107.3 |
| EIDD-02749 | HepG2 | >400 |
| EIDD-02749 | Huh-7 | >400 |
| EIDD-02749 | A-204 | >400 |
| EIDD-02749 | A549 | >400 |
| EIDD-02749 | BxPc3 | >400 |
| EIDD-02749 | H9c-2 | >400 |
| EIDD-02749 | IEC-6 | 371.7 |
| EIDD-02749 | Vero | >400 |

### Example 81. Synthesis of EIDD-02749-5′-Monophosphate (EIDD-02986

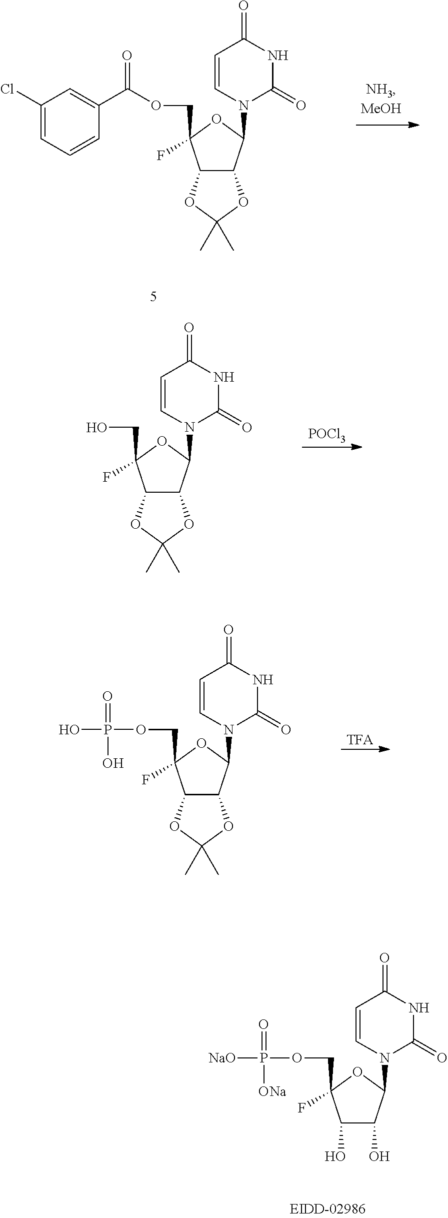

A heavy wall 350 mL round-bottomed pressure vessel was charged with 5′-(3-chlorobenzoyloxy)-4′-fluoro-2′,3′-O-isopropylideneuridine (4.1 g, 9.3 mmol) and 7N ammonia in methanol (66 mL, 462 mmol). The mixture was stirred for 6 h at room temperature after which time tlc indicated complete consumption of starting material. The mixture was concentrated in vacuo, and the resulting residue purified by column chromatography over silica gel (40 g) eluting with a methylene chloride/methanol gradient to give 4′-fluoro-2′,3′-O-isopropylideneuridine (2.5 g, 89%) as a white solid.

¹H NMR (400 MHz, Chloroform-d) δ 9.24 (s, 1H), 7.23 (d, J=8.0 Hz, 1H), 5.77 (d, J=8.0 Hz, 1H), 5.72 (s, 1H), 5.24 (dd, J=12.6, 6.5 Hz, 1H), 5.07 (dd, J=6.4, 1.3 Hz, 1H), 2.50 (s, 1H), 1.59 (s, 3H), 1.38 (s, 3H).

¹⁹F NMR (376 MHz, Chloroform-d) 6-115.53 (dd, J=12.4, 8.8 Hz).

A solution of tristriazolide in acetonitrile was freshly prepared by treating a mixture of 1,2,4-triazole (468.91 mg, 6.79 mmol) and triethylamine (0.95 mL, 6.79 mmol) in acetonitrile (7.5 mL) dropwise with phosphorus oxychloride (0.21 mL, 2.27 mmol) over a 5 min period at −15° C. After stirring for an additional 20 min at −15° C., the triethylammonium precipitate was removed by centrifuge, and the solution of tristriazolide was added to an acetonitrile solution (7.5 mL) of 4′-fluoro-2′,3′-O-isopropylideneuridine (225 mg, 0.74 mmol) at −15° C. After stirring for 15 min at −15° C., the mixture was allowed to warm to rt, and continued for another 1.5 hr. The mixture was quenched with 50 mM TEAB (30 mL), stirred for 1 h at rt, and concentrated to dryness in vacuo. The resulting residue was co-evaporated with water (2×20 mL) and purified by ion-exchange chromatography over DEAE-Sephadex A-25 (HCO₃⁻ form) eluting with a gradient from 0 to 0.2 M (700 mL) aqueous ammonium bicarbonate in 10% ethanol. Fractions were analyzed by tlc (7:2:1 iPa:NH₄OH:water), and target fractions combined and concentrated. The product was further purified by reversed-phase chromatography with a CombiFlash equipped with a C-18 column (50 g) eluting with 0.01M aqueous ammonium bicarbonate. Product containing fractions were pooled, frozen, and concentrated by lyophilization to give 4′-fluoro-2′,3′-O-isopropylideneuridine 5′-O-phosphate (131 mg, 46%) as a white solid.

¹H NMR (400 MHz, D₂O) δ 7.64 (d, J=8.0 Hz, 1H), 6.08 (s, 1H), 5.81 (d, J=7.8 Hz, 1H), 5.21 (dd, J=12.4, 6.6 Hz, 1H), 5.14 (d, J=6.5 Hz, 1H), 4.02-3.73 (m, 2H), 1.54 (s, 3H), 1.36 (s, 3H).

³¹P NMR (162 MHz, D₂O) δ 3.46.

¹⁹F NMR (376 MHz, D₂O) 6-113.90 (q, J=12.4, 11.9 Hz).

### 4′-Fluorouridine-5′-monophosphate (EIDD-02986)

A 50 mL round-bottomed flask was charge with 4′-fluoro-2′,3′-O-isopropylideneuridine-5′-O-phosphate (171 mg, 0.43 mmol), water (0.5 mL) and acetic acid (1.5 mL). The solution was cooled to 10° C. and treated with cold aqueous 90% trifluoroacetic acid (3.3 mL, 43.15 mmol). After 5 min, the mixture was allowed to warm to room temperature and stirred an additional 2 h. The mixture was concentrated in vacuo and the resulting gum co-evaporated with water (5×10 mL) followed by methanol (3×10 ml). The crude product as a solution in methanol (10 mL) was filtered, concentrated to approximately 4 mL in volume and treated with a cold solution of 1M sodium perchlorate in acetone (20 mL). After 20 min at 0° C., the white precipitate was collected by centrifuge. The white solid was washed with acetone (5×14 mL), dissolved in water (4 mL) and concentrated by lyophilization to give 4′-fluorouridine-5′-monophosphate (EIDD-02986) (78 mg, 45%) as the disodium form.

¹H NMR (400 MHz, D₂O) δ 7.75 (d, J=8.1 Hz, 1H), 6.09 (s, 1H), 5.92-5.82 (m, 1H), 4.58-4.49 (m, 1H), 4.42 (dd, J=6.4, 1.9 Hz, 1H), 4.11 (t, J=5.2 Hz, 3H).

³¹P NMR (162 MHz, D₂O) 6-0.27.

¹⁹F NMR (376 MHz, D₂O) 6-121.26 (dt, J=19.1, 5.1 Hz).

LCMS Calculated for C₉H₁₁FN₂O₉P [M−H⁺]: 341.0; found: 340.9.

### Example 82. Synthesis of EIDD-02749-5′-triphosphate (EIDD-02991)

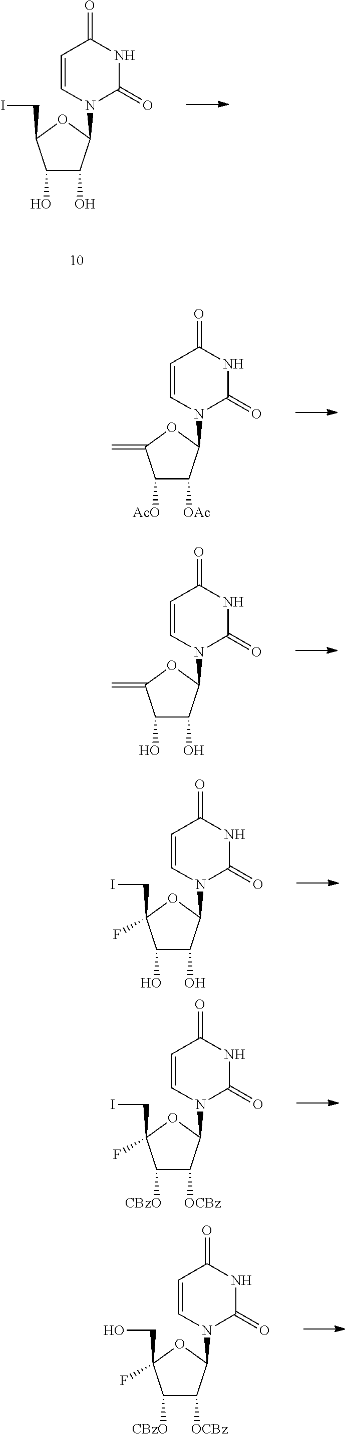

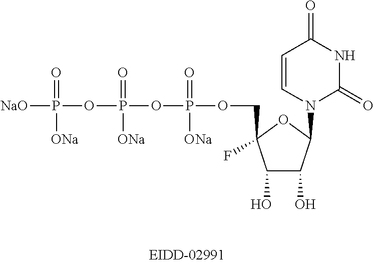

A 2 L three-necked round-bottomed flask flushed with argon and fitted with a mechanical stirrer, thermometer was charged with 5′-deoxy-5′-iodouridine (80 g, 225.92 mmol) and dry methanol (500 mL). Under argon atm, the white suspension was treated with a solution of 25% (4.37 M) sodium methoxide in methanol (103.4 mL, 451.85 mmol). The resulting homogeneous solution was stirred at 60° C. for 3 h. Methanol was removed in vacuo, and the resulting residue dissolved in anhydrous acetonitrile (300 mL). After addition of acetic anhydride (70.2 mL, 743 mmol), the mixture was heated to 60° C. for 5 h. Once cooled to room temperature, the mixture was concentrated in vacuo, and the resulting residue dissolved in ethyl acetate (500 mL) and treated with saturated sodium bicarbonate (100 mL). The organic layer was separated, washed with brine (100 mL), dried and concentrated to dryness to give 2′,3′-di-O-acetyl-4′,5′-didehydro-5′-deoxyuridine (70 g, 99% yield).

¹H NMR (400 MHz, DMSO-d₆) δ 11.53 (d, J=1.9 Hz, 1H), 7.75 (d, J=8.1 Hz, 1H), 6.07 (d, J=4.3 Hz, 1H), 5.92 (d, J=6.5 Hz, 1H), 5.69 (dd, J=8.0, 1.8 Hz, 1H), 5.63 (dd, J=6.4, 4.3 Hz, 1H), 4.52 (t, J=1.9 Hz, 1H), 4.28 (d, J=2.4 Hz, 1H), 2.08 (s, 3H), 2.04 (s, 3H).

In a 1 L round-bottomed flask, a solution of 2′,3′-di-O-acetyl-4′,5′-didehydro-5′-deoxyuridine (70 g, 225.6 mmol) in methanol (350 mL) was treated with 30% ammonium hydroxide (85.3 mL, 2190.7 mmol). After 18 h at room temperature, the mixture was concentrated in vacuo and the resulting residue dissolved in a 65:35:5 mixture of acetonitrile:isopropanol:methanol. After 30 min, the white precipitate was collected by vacuum filtration and washed with acetonitrile and hexanes. A second crop was isolated by concentrating the filtrate and stirring the resulting solid with acetonitrile. Combined crops were dried under high vacuum for 18 h to give 4′,5′-didehydro-5′-deoxyuridine (35 g. 68% yield) as a white solid.

¹H NMR (400 MHz, DMSO-d₆) δ 11.44 (s, 1H), 7.59 (d, J=8.1 Hz, 1H), 5.96 (d, J=5.4 Hz, 1H), 5.64 (d, J=8.1 Hz, 1H), 5.60 (d, J=5.8 Hz, 1H), 5.46 (d, J=5.7 Hz, 1H), 4.38 (t, J=5.5 Hz, 1H), 4.33 (s, 1H), 4.24 (q, J=5.5 Hz, 1H), 4.17 (d, J=1.8 Hz, 1H).

A 2 L three-necked round-bottomed flask was charged with 4′,5′-didehydro-5′-deoxyuridine (35 g, 154.7 mmol) and anhydrous acetonitrile (400 mL). The suspension was cooled to 0° C. under argon atm and treated with triethylamine trihydrofluoride (12.6 mL, 77.4 mmol) followed by the addition of N-iodosuccinimide (45.3 g, 201.2 mmol). After 1 h at 0° C., tlc (10% methanol in methylene chloride) indicated complete conversion. While still cold, the mixture was vacuum filtered. The isolated solid was washed sequentially with acetonitrile, dichloromethane, hexanes, and then dried under high vacuum for 18 h to give 5′-deoxy-4′-fluoro-5′-iodouridine (35 g, 61%).

¹H NMR (400 MHz, Methanol-d₄) δ 7.77 (d, J=8.1 Hz, 1H), 6.05 (s, 1H), 5.69 (d, J=8.1 Hz, 1H), 4.43 (dd, J=18.2, 6.5 Hz, 1H), 4.25 (d, J=6.6 Hz, 1H), 3.85-3.63 (m, 2H).

¹⁹F NMR (376 MHz, Methanol-d₄) δ −112.49 (ddd, J=20.9, 18.1, 6.1 Hz).

A 150 mL round-bottomed flask was charged with 5′-deoxy-5′-iodo-4′-fluorouridine (2.6 g, 6.99 mmol) and methylene chloride (35 mL). After stirring for 20 min at room temperature, the suspension was cooled to 0° C. and treated with benzyl chloroformate (4.49 mL, 31.44 mmol) followed by dropwise addition of 1-methylimidazole (3.34 mL, 41.93 mmol) over a 10 min period. The mixture was stirred an additional 10 min at 0° C. and then allowed to slowly warm to room temperature. After 18 h, the turbid mixture was diluted with methylene chloride (120 mL) and washed with 0.5M HCl solution (75 mL), water (50 mL), and brine (50 mL). The organic layer was separated, dried and concentrated in vacuo. The resulting residue was purified by column chromatography over silica gel (80 g) eluting with a methylene chloride/methanol gradient. Pure fractions were combined and concentrated in vacuo to give 2′,3′-di-O-benzyloxycarbonyl-5′-deoxy-4′-fluoro-5′-iodouridine (4.2 g, 94% yield) as a white solid.

¹H NMR (400 MHz, CDCl₃) δ 9.02 (s, 1H), 7.44-7.28 (m, 10H), 7.14 (d, J=8.0 Hz, 1H), 5.86-5.72 (m, 2H), 5.69-5.57 (m, 2H), 5.19 (d, J=4.3 Hz, 2H), 5.09 (d, J=3.1 Hz, 2H), 3.71-3.35 (m, 2H).

¹⁹F NMR (376 MHz, CDCl₃) δ −107.06 (td, J=18.6, 7.3 Hz).

In a 100 mL round-bottomed flask a 55% tetrabutylammonium hydroxide solution in water (8.04 mL, 9.37 mmol) was adjusted to pH 3.5 by dropwise addition of trifluoroacetic acid (0.72 mL, 9.37 mmol) while maintaining a temperature below 25° C. The mixture was then treated with a methylene chloride (15 mL) solution of 2′,3′-di-O-benzyloxycarbonyl-5′-deoxy-4′-fluoro-5′-iodouridine (2 g, 3.12 mmol) followed by addition of 3-chloroperbenzoic acid (3.6 g, 15.62 mmol) in portions over a 30 min period. After one hour the pH drifted to pH 1.4. The mixture was adjusted back to pH 3.5 with 1N sodium hydroxide and allowed to stir for 16 h after which time tlc (10% methanol in methylene chloride) and LCMS indicated complete conversion. The reaction mixture was quenched by addition of sodium thiosulfate (3.21 g, 20.31 mmol) slowly in portions while maintaining a temperature below 25° C. After stirring for 30 min, the methylene chloride layer was separated, and the aqueous layer extracted with additional methylene chloride (2×30 mL).

Combined organic layers were dried over sodium sulfate, concentrated, and purified by column chromatography over silica gel (80 g) eluting with 60% ethyl acetate in hexanes followed by a second column of silica gel (80 g) eluting with a methylene chloride/methanol gradient to give 2′,3′-di-O-benzyloxycarbonyl-4′-fluorouridine (1.05 g, 63% yield) as a white solid.

¹H NMR (400 MHz, CDCl₃) δ 9.30 (s, 1H), 7.39-7.29 (m, 10H), 7.21 (d, J=8.1 Hz, 1H), 5.83 (dd, J=17.8, 7.0 Hz, 1H), 5.77-5.71 (m, 2H), 5.61 (dd, J=7.0, 2.4 Hz, 1H), 5.17 (d, J=4.8 Hz, 2H), 5.09 (s, 2H), 3.86 (q, J=5.8, 4.9 Hz, 2H), 3.06 (s, 1H).

¹⁹F NMR (376 MHz, CDCl₃) δ −121.03 (dt, J=17.7, 4.6 Hz). 4′-Fluorouridine 5′-O-triphosphate (EIDD-02991) A 10 mL round-bottomed flask charged with 2′,3′-di-O-benzyloxycarbonyl-4′-fluorouridine (348 mg, 0.66 mmol) and anhydrous trimethyl phosphate (3.5 mL). After stirring for 20 min at room temperature, the solution was cooled to 0° C. and treated with 1-methyl-imidazole (115 μL, 1.44 mmol) followed by dropwise addition of phosphorus oxychloride (122 μL, 1.31 mmol) over a 40 min period. The mixture continued to stir at 0° C. for 3.5h after which time tlc (10% methanol in DCM and then 7:2:1 iPa:NH₄OH:water) indicated complete phosphorylation. The mixture was treated with tributylamine (0.94 mL, 3.94 mmol), tris(tetrabutylammonium)pyrophosphate (887 mg, 0.98 mmol), and anhydrous DMF (1.5 mL). After 1 h at room temperature, the reaction mixture was quenched with 100 mM TEAB (20 mL), stirred for 1 h, degassed by pump-fill with argon (3×) and treated with 10% palladium on carbon (100 mg). After cooling with an ice-bath, the mixture was pump-filled with hydrogen (2×) followed by vigorous stirring under atm pressure of hydrogen for 30 min. The mixture was pump-filled with argon and then vacuum filtered through a pad of Celite. The palladium was washed with water (2×20 mL). Combined filtrates were washed with ether (4×60 mL) and then concentrated in vacuo at 25° C. The residue was co-evaporated with water (2×25 mL) and purified by column chromatography over DEAE-Sephadex GE A-25 (10 mm×130 mm) eluting with a gradient from 100 mM to 500 mM TEAB (900 mL). Pure fractions as determined by tlc (8:1:1 NH₄OH:iPrOH:water) were combined and concentrated in vacuo with the bath temperature set at 25° C. The resulting solid was dissolved in methanol (1 mL) and treated with saturated solution of sodium perchlorate in acetone (10 mL). The resulting white precipitate was collected by centrifuge and washed with acetone (5×5 mL).The solid was dissolved in water (1 mL), frozen and lyophilized to yield 4′-fluorouridine 5′-O-triphosphate (3.14 mg, 0.81% yield) as the tetrasodium form.

¹H NMR (400 MHz, D₂O) δ 7.77 (d, J=8.0 Hz, 1H), 6.15 (d, J=1.9 Hz, 1H), 5.91 (d, J=8.1 Hz, 1H), 4.72-4.57 (m, 1H), 4.41 (d, J=6.3 Hz, 1H), 4.30 (ddd, J=10.2, 6.3, 3.0 Hz, 1H), 4.17 (dt, J=10.8, 5.0 Hz, 1H).

³¹P NMR (162 MHz, D₂O) 6-7.81(d), −11.84 (d, J=19.2 Hz), −22.23 (t).

¹⁹F NMR (376 MHz, D₂O) 6-121.09 (unresolved dt, J=19.2 Hz).

LCMS Calculated for C₉H₁₃FN₂O₁₅P₃[M−H⁺]: 500.9; found: 500.8.

### Example 83. Synthesis of EIDD-02749-5′-Isobutyl ester (EIDD-02947)

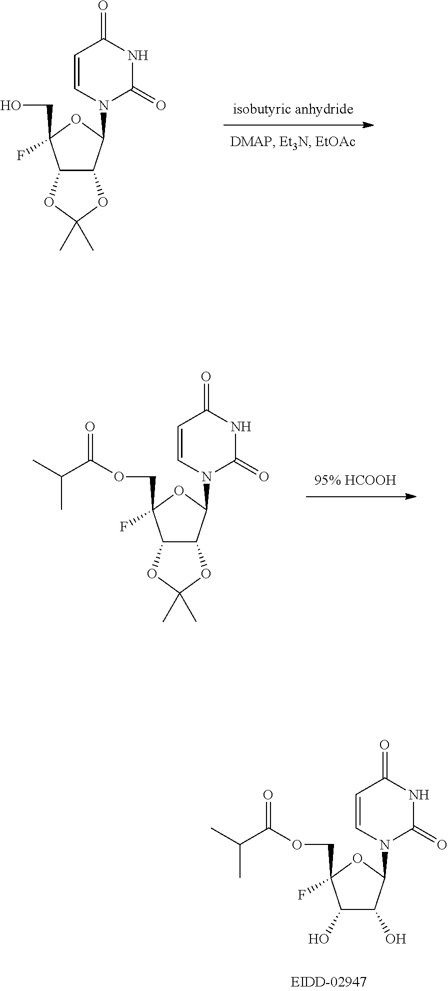 [SMILES: CC1(C)O[C@H]2[C@H](n3ccc(=O)[nH]c3=O)O[C@](F)(CO)[C@H]2O1 ; CC(C)C(=O)OC[C@@]1(F)O[C@@H](n2ccc(=O)[nH]c2=O)[C@@H]2OC(C)(C)O[C@@H]21 ; CC(C)C(=O)OC[C@@]1(F)O[C@@H](n2ccc(=O)[nH]c2=O)[C@H](O)[C@@H]1O]

To a 25 mL pear-shaped flask charged with 1(3aS, 4S)-4-fluoro-4-(hydroxymethyl)-2,2-dimethyl-6,6a-dihydro-3aH-furo[3,4-d][1,3]dioxol-6-yl]pyrimidine-2,4-dione (0.1 g, 0.33 mmol) and DMAP (2.0 mg, 0.02 mmol) was added EtOAc (1.1 mL) to give a colorless solution. The vessel was vacuumed and charged with argon. Then Et₃N (083.12 mL, 0.83 mmol) was added, followed by isobutyric anhydride (0.07 mL, 0.4 mmol). This reaction solution was allowed to stir at room temperature overnight. After overnight stirring, TLC showed no SM. The reaction solution was transferred into a separation funnel and water was added. The aqueous layer was separated and re-extracted with DCM once. The combined organic layers was dried (Na₂SO₄), filtered and concentrated in vacuo. The crude material was purified by ISCO column chromatography (12 g) eluting from 100% hexanes to 80% EtOAc in hexanes to afford [(3aS, 4S)-6-(2,4-dioxopyrimidin-1-yl)-4-fluoro-2,2-dimethyl-6,6a-dihydro-3aH-furo[3,4-d][1,3]dioxol-4-yl]methyl 2-methylpropanoate (0.11 g, 89%) as a white glassy solid. To a 25 mL pear-shaped flask charged with [(3aS, 4S)-6-(2,4-dioxopyrimidin-1-yl)-4-fluoro-2,2-dimethyl-6,6a-dihydro-3aH-furo[3,4-d][1,3]dioxol-4-yl]methyl 2-methylpropanoate (0.11 g, 0.3000 mmol) was added 95% formic acid (12 mL, 0.3 mmol) to give a colorless solution. After stirring at room temperature for 3.5 h, solvent was removed in vacuo. Then water and Celite were added, concentrated in vacuo. The crude material was purified by ISCO column chromatography (12 g) eluting from 100% DCM to 15% MeOH in DCM to afford the product with some impurity. This material was re-purified by ISCO column chromatography (12 g) eluting from 100% hexanes to 100% EtOAc to afford [(2S, 3S)-5-(2,4-dioxopyrimidin-1-yl)-2-fluoro-3,4-dihydroxy-tetrahydrofuran-2-yl]methyl 2-methylpropanoate (EIDD-02947) (7.8 mg, 8% yield) after lyophilizing overnight as a white fluffy solid.

¹H NMR (400 MHz, Methanol-d₄) δ 7.59 (d, J=8.1 Hz, 1H), 5.87 (d, J=2.1 Hz, 1H), 5.69 (d, J=8.1 Hz, 1H), 4.51 (dd, J=19.2, 6.9 Hz, 1H), 4.44-4.34 (m, 2H), 4.28 (dd, J=11.9, 8.1 Hz, 1H), 2.72-2.53 (m, 1H), 1.17 (dd, J=7.0, 4.8 Hz, 6H).

¹⁹F NMR (376 MHz, Methanol-d₄) δ −123.39 (dt, J=19.1, 8.0 Hz). ¹³C NMR (101 MHz, CD₃OD) δ 176.24, 164.59, 150.18, 142.88, 142.80, 116.65, 114.36, 101.82, 101.62, 95.71, 95.46, 70.98, 70.88, 70.27, 70.07, 61.44, 61.02, 33.66, 33.51, 17.90, 17.84, 17.80.

### Example 84. Synthesis of EIDD-02749-5′-L-Valine ester (EIDD-02971)

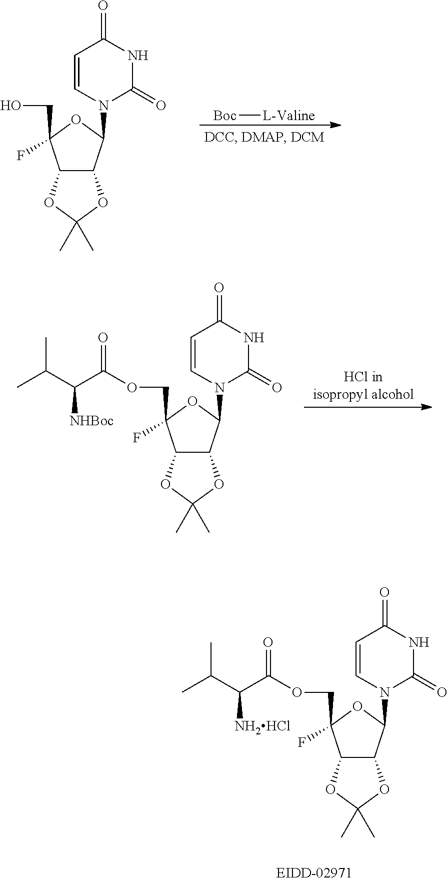

To a 25 mL pear-shaped flask charged with 1-[(3aS, 4S, 6R, 6aR)-4-fluoro-4-(hydroxymethyl)-2,2-dimethyl-6,6a-dihydro-3aH-furo[3,4-d]1[1,3]dioxol-6-yl]pyrimidine-2,4-dione (0.15 g, 0.5 mmol), Boc-L-Valine (0.13 g, 0.6 mmol) and DMAP (0.01 g, 0.05 mmol) was added dry DCM (2 mL) to give a colorless solution. The reaction vessel was vacuumed and charged with argon. Then DCC (0.12 g, 0.6 mmol) was added all at once to give a white suspension. After overnight stirring, the white suspension was filtered through Celite and the solids were washed with DCM. Celite was added to the filtrate, and the filtrate was then concentrated in vacuo. The crude material was purified by ISCO column chromatography (24 g) eluting from 100% hexanes to 100% EtOAc to afford [(3aS, 4S, 6R, 6aR)-6-(2,4-dioxopyrimidin-1-yl)-4-fluoro-2,2-dimethyl-6,6a-dihydro-3aH-furo[3,4-d][1,3]dioxol-4-yl]methyl (2S)-2-(tert-butoxycarbonylamino)-3-methyl-butanoate (0.158 g, 63%).

To a 10 mL pear-shaped vial charged with [(3aS, 4S, 6R, 6aR)-6-(2,4-dioxopyrimidin-1-yl)-4-fluoro-2,2-dimethyl-6,6a-dihydro-3aH-furo[3,4-d][1,3]dioxol-4-yl]methyl (2S)-2-(tert-butoxycarbonylamino)-3-methyl-butanoate (50 mg, 0.1 mmol) was added isopropyl acetate (1.3 mL) to give a colorless solution under argon. This was cooled to 0° C. and then 5-6 N HCl in IPA (0.05 mL) was added dropwisely. After 1.5 h, TLC showed mainly SM. Then more 5-6 N HCl in IPA (0.05 mL) was added and this was put in the fridge overnight. The next day, some solids formed in the flask. This was filtered through a medium sintered glass frit and washed with Et₂O. Since the solids were hygroscopic, it was dissolved in MeOH and concentrated in vacuo. The previous mother liquor had contained added solids, which were filtered, dissolved in MeOH, and combined with the previous solution. Concentration in vacuo gave the product with an impurity. This was re-dissolved in EtOH and triturated with Et₂O. After stirring for a while, this mixture was filtered and the solids were dissolved in EtOH. Then more Et₂O was added and the solids were filtered after stirring. Finally, the solids were dissolved in MeOH, concentrated in vacuo, dissolved in water and lyophilized overnight to afford [(1S)-1-[[(2S, 3S, 4R, 5R)-5-(2,4-dioxopyrimidin-1-yl)-2-fluoro-3,4-dihydroxy-tetrahydrofuran-2-yl]methoxycarbonyl]-2-methyl-propyl]ammonium chloride (EIDD-02971) (11 mg, 30%) as light yellow solids.

### Example 85. Synthesis of EIDD-02749-2′, 3′, 5′-Isoburyl triester (EIDD-02954)

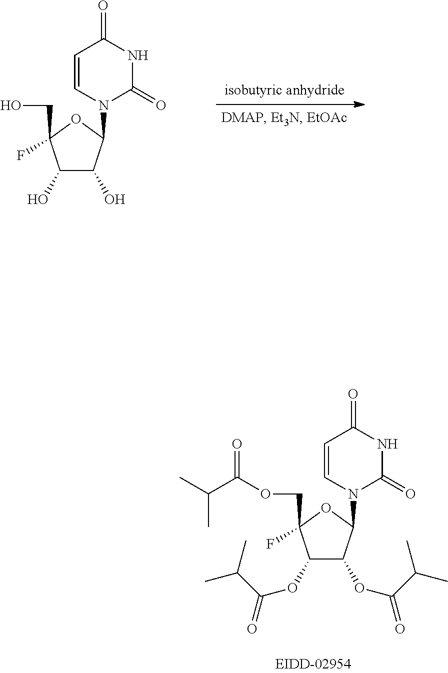 [SMILES: O=c1ccn([C@@H]2O[C@](F)(CO)[C@@H](O)[C@H]2O)c(=O)[nH]1 ; CC(C)C(=O)OC[C@@]1(F)O[C@@H](n2ccc(=O)[nH]c2=O)[C@H](OC(=O)C(C)C)[C@@H]1OC(=O)C(C)C]

To a 50 mL rbf charged with 1-[(2R, 3R, 4S, 5S)-5-fluoro-3,4-dihydroxy-5-(hydroxymethyl)tetrahydrofuran-2-yl]pyrimidine-2,4-dione (68 mg, 0.26 mmol) and DMAP (6.3 mg, 0.05 mmol) was added EtOAc (2.6 mL) to give a suspension. This was vacuumed and charged with argon. Then Et₃N (0.18 mL, 1.3 mmol) was added. The flask was cooled to 0° C. and isobutyric anhydride (0.15 mL, 0.91 mmol) was added dropwisely. After 15 min, the resulting colorless solution was allowed to stir at room temperature. After 3.5 h, TLC showed no SM. Then water was added dropwisely. After stirring for 5 min, the reaction mixture was transferred into a separation funnel and more EtOAc was added. The organic layer was separated, dried (Na₂SO₄), filtered and concentrated in vacuo with Celite. The crude material was purified by ISCO column chromatography (24 g) eluting from 100% hexanes to 100% EtOAc to afford [(2S, 3S, 4R, 5R)-5-(2,4-dioxopyrimidin-1-yl)-2-fluoro-3,4-bis(2-methylpropanoyloxy)tetrahydrofuran-2-yl]methyl 2-methylpropanoate (EIDD-02954) (0.1 g, 82%) as white solids.

¹H NMR (400 MHz, Methanol-d₄) δ 7.62 (d, J=8.0 Hz, 1H), 5.93-5.78 (m, 2H), 5.69 (d, J=7.9 Hz, 1H), 5.64 (dd, J=7.2, 2.1 Hz, 1H), 4.35 (dd, J=7.8, 3.5 Hz, 2H), 2.68-2.57 (m, 3H), 1.26-1.07 (m, 18H).

¹⁹F NMR (376 MHz, Methanol-d₄) δ −120.16 (dt, J=19.6, 7.8 Hz).

### Example 86. Synthesis of 4′-fluoro-4-thiouridine

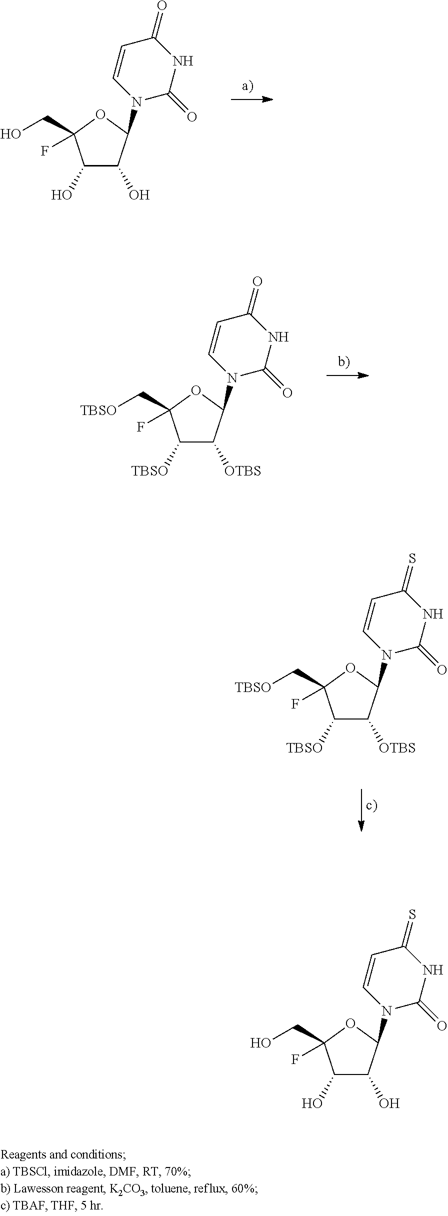 [SMILES: O=c1ccn([C@@H]2O[C@](F)(CO)[C@@H](O)[C@H]2O)c(=O)[nH]1 ; CC(C)(C)[Si](C)(C)OC[C@@]1(F)O[C@@H](n2ccc(=O)[nH]c2=O)[C@H](O[Si](C)(C)C(C)(C)C)[C@@H]1O[Si](C)(C)C(C)(C)C ; CC(C)(C)[Si](C)(C)OC[C@@]1(F)O[C@@H](n2ccc(=S)[nH]c2=O)[C@H](O[Si](C)(C)C(C)(C)C)[C@@H]1O[Si](C)(C)C(C)(C)C ; O=c1[nH]c(=S)ccn1[C@@H]1O[C@](F)(CO)[C@@H](O)[C@H]1O]

### Preparation of 2′,3′,5′-tri-O-(t-butyldimethylsilyl)-4′-fluoro-uridine

To a solution of 4′-fluoro-uridine (500 mg, 1.9 mmol) in DMF (20 ml) taken in 100 ml RBF, TBDMSCl (1.2 gm, 7.6 mmol) and imidazole (650 mg, 9.5 mmol) were added under inert atmosphere at 0° C. and continued stirring at room temperature. After completion, the reaction mixture was concentrated under reduced pressure and the crude product was dissolved in dichloromethane and washed with saturated aq. NaHCO₃ followed by brine. The combined organic layers were dried over Na₂SO₄, filtered, and concentrated under reduced pressure. The residue was purified by chromatography. Product was obtained as colorless foam (yield 58%).

### Preparation of 2′,3′,5′-tri-O-(t-butyldimethylsilyl)-4′-fluoro-4-thiouridine

To a solution of 2′,3′,5′-tri-O-(t-butyldimethylsilyl)-4′-fluoro-uridine (600 mg, 1 mmol) in anhydrous THE (20 ml), Lawesson's reagent (freshly purchased) (590 mg, 1.5 mmol) and potassium carbonate (29 mg, 0.2 mmol) were added and the reaction mixture was refluxed for 5 hr. After completion the reaction mixture was concentrated under reduced pressure and the crude product was purified by column chromatography. Product was obtained as colorless foam (yield 52%).

### Preparation of 4′-fluoro-4-thiouridine

To a solution of 2′,3′,5′-tri-O-(t-butyldimethylsilyl)-4′-fluoro-4-thiouridine (250 mg, 0.41 mmol) in anhydrous tetrahydrofuran (5 ml), 1M solution of tetrabutylammonium fluoride (2 ml) was added and stirred at room temperature for 5 hr. After completion, the reaction mixture was concentrated under reduced pressure and the crude product was purified by silica gel column chromatography. 1H NMR 400 MHz, CD₃OD, δ 7.77 (1H, d, J=8 Hz), 6.06 (1H, d, J=4 Hz), 5.69 (1H, d, J=8 Hz), 4.42 (1H, dd, J=6.4 Hz, 20 Hz), 4.25 (1H, dd, 6.4 Hz, 2.4 Hz), 3.73 (2H, m); 19F NMR 376 MHz 6-123.57, (1F, dt, J=18.8 Hz, 3.7 Hz)

### Example 87. Synthesis of 1′-deutero-4′-fluorouridine

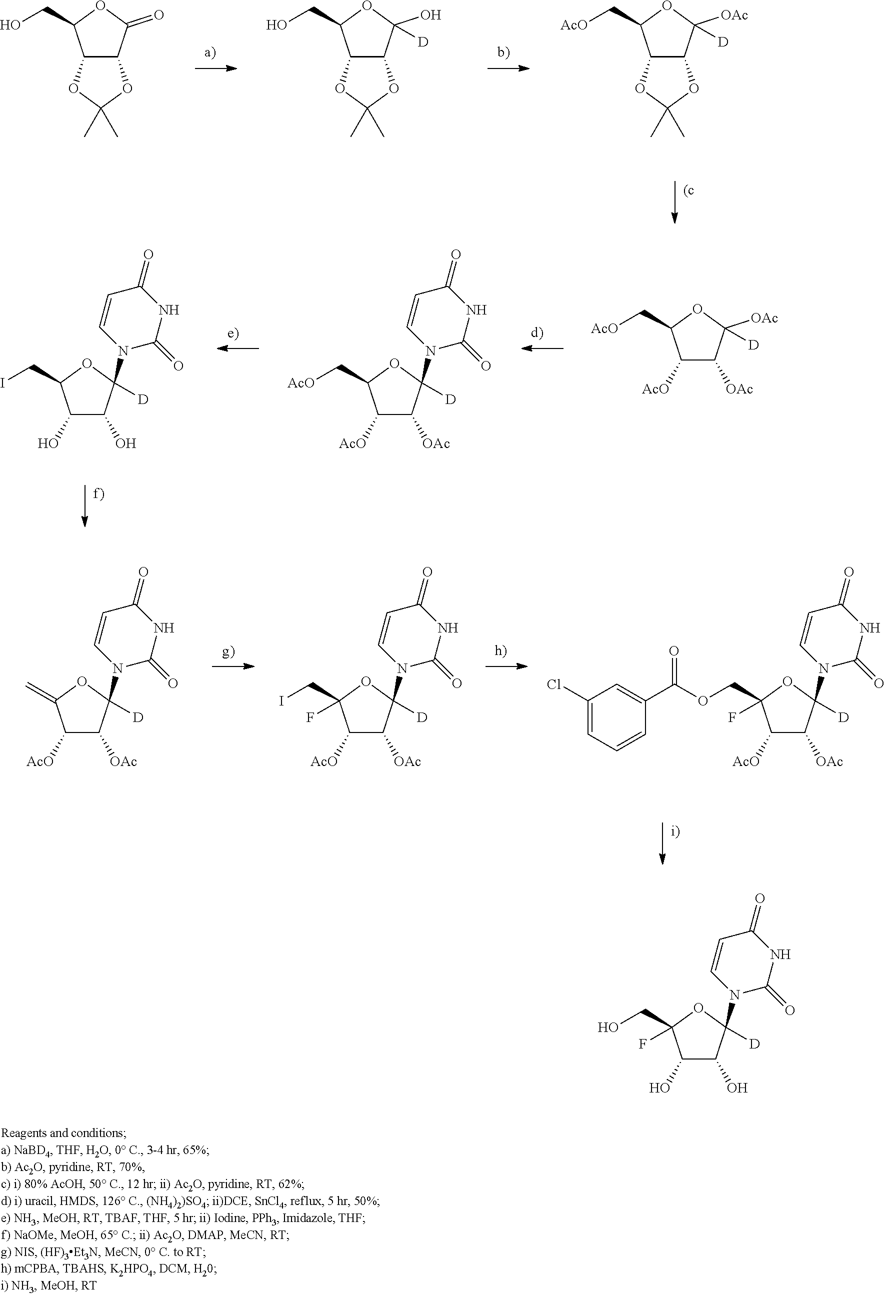

### Preparation of 1-deutero-2,3-O-isopropylidene-D-ribofuranose

To a solution of 2,3-O-isopropylidene-D-ribonolactone (3 g, 16 mmol) in 9:1 (THE: H2O) (50 ml), taken in a 250 ml RBF, NaBD₄ (1 g, 24 mmol) was added slowly in portions at 0° C. with continued stirring. After completion, the reaction mixture was quenched with acetone and stirred at room temperature for additional 30 min. The reaction mixture was diluted with excess ethyl acetate (100 ml) and washed with saturated aq. NH₄Cl followed by saturated aq. NaHCO₃ and brine. The combined organic layers were dried over Na₂SO₄, filtered, and concentrated under reduced pressure. The residue was purified by chromatography. The product was obtained as colorless oil (yield 65%).

### Preparation of 1,5-di-O-acetyl-1-deutero-2,3-O-isopropylidene-D-ribofuranose

To a solution of 1-deutero-2,3-O-isopropylidene-D-ribofuranose (1.9 g, 10 mmol) in DCM (50 ml), acetic anhydride (2.4 ml, 25 mmol), trimethylamine (4.2 ml, 30 mmol), and DMAP (195 mg, 1.6 mmol) were added at 0° C. Stirring continued at room temperature. After completion, the reaction mixture was washed with saturated aq. NH₄Cl followed by saturated aq. NaHCO₃ (twice) and brine. The combined organic layers were dried over Na₂SO₄, filtered, and concentrated under reduced pressure. The residue was purified by chromatography. Product was obtained as colorless syrup (yield 70%).

### Preparation of 1,2,3,5-tetra-O-acetyl-1-deutero-D-ribofuranose

1,5-di-O-acetyl-1-deutero-2,3-O-isopropylidene-D-ribofuranose (2 g, 7.2 mmol) was dissolved in 80% acetic acid (50 ml) in 100 ml RBF and stirred at 50° C. for 12 hr. After completion, the reaction mixture was concentrated under reduced and co-evaporated with toluene twice. The crude product was dissolved in pyridine (20 ml). Acetic anhydride (1.7 ml, 18 mmol) and DMAP (122 mg, 1 mmol) were added at 0° C. and stirring continued at room temperature. After completion, the reaction mixture was concentrated under reduced pressure and the crude product was dissolved in dichloromethane and washed with 5% aq. HCl followed by saturated aq. NaHCO₃ and brine. The combined organic layers were dried over Na₂SO4, filtered, and concentrated under reduced pressure. The residue was purified by chromatography. Product was obtained as syrup which crystallizes upon standing (yield 62% for 2 steps).

### Preparation of 2′,3′,5′-tri-O-acetyl-1′-deutero-uridine

To a suspension of uracil (670 mg, 6 mmol) in HMDS (10 ml) in a 100 ml RBF, catalytic ammonium sulfate was added and refluxed at 126° C. under inert atmosphere for 12 hr. The reaction mixture cooled down and concentrated under reduced pressure. The residue was subjected to high vacuum and charged with anhydrous acetonitrile, compound 1,2,3,5-tetra-O-acetyl-1-deutero-D-ribofuranose (950 mg, 3 mmol) in acetonitrile and tin tetrachloride (350 μL, 3 mmol). The reaction mixture was refluxed under inert atmosphere for 5 hr. After completion, the reaction mixture was quenched with solid NaHCO₃ and Celite and stirred at room temperature for 30 min. A few drops of saturated aq. NaHCO₃ and continued stirring for 2-3 hr. The white precipitate formed was filtered and washed with DCM, the filtrate was concentrated under reduced pressure and purified by column chromatography. Product was obtained as colorless solid (yield 50%).

### Preparation of 5′-deoxy-1′-deutero-5′-iodo-uridine

To a solution of 2′,3′,5′-tri-O-acetyl-1′-deutero-uridine (745 mg, 2 mmol) in methanol (10 ml), 7N ammonia in methanol was added and stirred at room temperature. After completion, the reaction mixture was concentrated under reduced pressure. The crude product was triturated with ethyl acetate and the resulting solid was taken in a 100 ml RBF and suspended in THE. Triphenylphosphine (786 mg, 3 mmol), imidazole (200 mg, 3 mmol) and iodine (600 mg, 2.3 mmol) were added and stirred at room temperature for 8 hr. After completion, the reaction mixture was concentrated under reduced pressure and the residue was stirred with isopropanol. The colorless solid formed was filtered and dried (yield 45%).

### Preparation of Compound 2′,3′-di-O-acetyl-1′-deutero-5′-deoxy-4′,5′-didehydrouridine

To a solution of 5′-deoxy-1′-deutero-5′-iodo-uridine (530 mg, 1.5 mmol) in methanol, sodium methoxide 25% by weight in methanol (325 μL) was added and stirred at 65° C. under inert atmosphere. After completion, the reaction mixture was concentrated under reduced pressure. The crude product was taken in MeCN (10 ml) and treated with acetic anhydride (425 μL, 4.5 mmol) and DMAP (20 mg, 0.15 mmol) and stirred at room temperature for 12 hr. After completion, the reaction mixture was quenched with saturated aq. NaHCO₃, diluted with DCM, washed with saturated aq. NaHCO₃ and brine. The combined organic layers were dried over Na₂SO4, filtered, and concentrated under reduced pressure. The residue was purified by chromatography yielding product as colorless solid.

### g) Preparation of Compound 2′,3′-di-O-acetyl-1′-deutero-5′-deoxy-5′-iodo-4′-fluorouridine

To a solution of compound 2′,3′-di-O-acetyl-1′-deutero-5′-deoxy-4′,5′-didehydrouridine (460 mg, 2 mmol) in anhydrous acetonitrile (5 ml) in 50 ml RBF, triethylamine trihydrofluoride (162 μL, 1 mmol) and N-iodosuccinimide (2.6 mmol) were added at 0° C. After 60 min, the reaction mixture was slowly warmed to room temperature. After completion, the reaction mixture was concentrated under reduced pressure and purified by column chromatography.

### h) Preparation of Compound 2′,3′-di-O-acetyl-1′-deutero-5′-m-chlorobenzoate-4′-fluorouridine

To a solution of 2′,3′-di-O-acetyl-1′-deutero-5′-deoxy-5′-fluoro-4′-iodouridine (460 mg, 1 mmol) in 5:1 (DCM:H₂O) (50 ml) in a 100 ml RBF, tetrabutylammonium hydrogen sulfate (370 mg, 1.1 mmol) and potassium phosphate dibasic (260 mg, 1.5 mmol) were added, and the reaction mixture was cooled to 0° C. meta-chloroperbenzoic acid (860 mg, 4 mmol) was added slowly in portions and reaction mixture was allowed to warm to room temperature and vigorous stirring was continued for another 12 hr. After completion, the reaction mixture was quenched with aq. Na₂SO₃ and diluted with DCM (30 ml). The organic layer was separated and washed with saturated aq. NaHCO₃ and brine. The combined organic layers were dried over Na₂SO₄, filtered, and concentrated under reduced pressure. The residue was purified by chromatography.

### i) Preparation of 1′-deutero-4′-fluorouridine

To a solution of 2′,3′-di-O-acetyl-1′-deutero-5′-m-chlorobenzoate-4′-fluorouridine (250 mg, 0.5 mmol) in methanol (10 ml), 7N ammonia in methanol (2 ml) was added and stirred at room temperature. After completion, the reaction mixture was concentrated under reduced pressure and purified by column chromatography.

### Example 88. Synthesis of 4′-fluoro-carbauridine

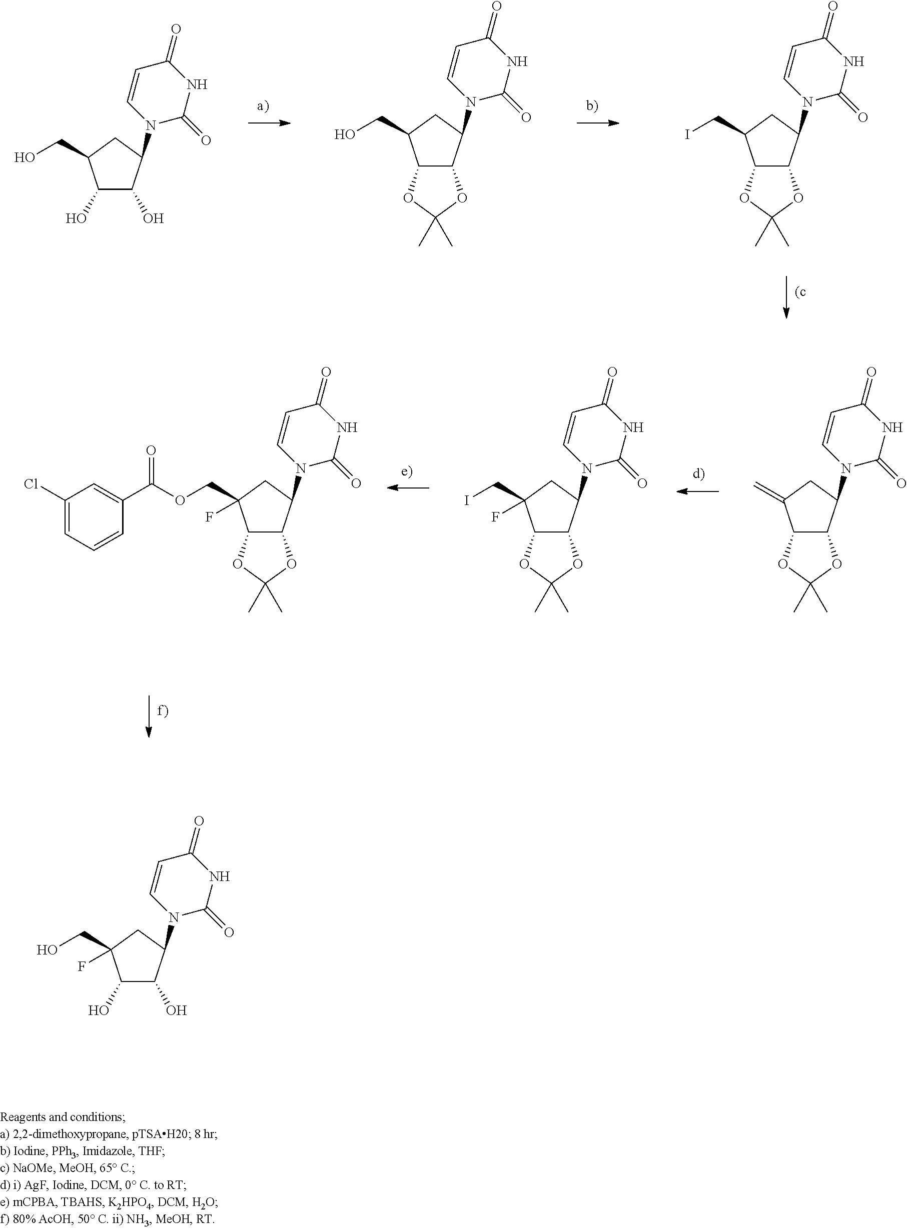

To a suspension of compound carbauridine (2.5 gm, 10 mmol) in anhydrous acetone (200 ml), 2,2-dimethoxypropane (1.2 ml, 10 mmol) and concentrated sulfuric acid (200 QL, 2 mmol) were added at 0° C. under inert atmosphere and stirring continued at room temperature. After completion, the reaction mixture was quenched with NaHCO₃, stirred for 30 min, and was filtered. The filtrate was concentrated under reduced pressure and the residue was purified by column chromatography.

To a solution of the above synthesized acetonide (1.4 gm, 5 mmol) in THE (100 ml) in a 250 ml RBF, triphenylphosphine (2 gm, 7.5 mmol), imidazole (500 mg, 7.5 mmol) and iodine (1.4 gm, 5.5 mmol) were added at 0° C. under inert atmosphere and stirred at room temperature for 8 hr. After completion, the reaction mixture was concentrated under reduced pressure and the residue was taken up in isopropanol (100 ml) and stirred at room temperature. The colorless solid formed was filtered and dried.

To a solution of the iodo compound synthesized above (1 gm, 2.5 mmol) in methanol, sodium methoxide 25% by weight in methanol (1.1 ml, 5 mmol) was added and stirred at 65° C. under an inert atmosphere. After completion, the reaction mixture was concentrated under reduced pressure. The crude product was dissolved in DCM (100 ml) and filtered through a Celite bed. The filtrate was concentrated under reduced pressure and the residue was purified by column chromatography.

To a solution of the alkene product obtained above (800 mg, 3 mmol) in anhydrous DCM (50 ml) in a 100 ml RBF, silver fluoride (950 mg, 7.5 mmol) was added followed by dropwise addition of iodine (1.5 gm, 6 mmol) in THF. After addition, the reaction mixture was slowly allowed to warm to room temperature and stirred for additional 30 min at room temperature. After completion, the reaction mixture was filtered through a Celite bed and the filtrate was washed with saturated aq. Na₂S203 followed by saturated aq. NaHCO₃ and brine. The combined organic layers were dried over Na₂SO₄, filtered, and concentrated under reduced pressure. The residue was purified by chromatography.

To a solution of the above compound (610 mg, 1.5 mmol) in 5:1 (DCM:H₂O) (50 ml) in a 100 ml RBF, tetrabutylammonium hydrogen sulfate (560 mg, 1.65 mmol) and potassium phosphate dibasic (400 mg, 2.3 mmol) were added. The reaction mixture was cooled to 0° C. m-Chloroperbenzoic acid (1.0 gm, 6 mmol) was added slowly in portions, and the reaction mixture was allowed to warm to room temperature. Vigorous stirring was continued for another 12 hr. After completion, the reaction mixture was quenched with aq.

Na₂SO₃ and diluted with DCM (50 ml). The organic layer was separated and washed with saturated aq. NaHCO₃ and brine. The combined organic layers were dried over Na₂SO₄, filtered, and concentrated under reduced pressure. The residue was purified by chromatography.

The above compound (450 mg, 1 mmol) was taken up in 80% acetic acid (20 ml) and stirred at 50° C. for 12 hr. After completion, the reaction mixture was concentrated under reduced pressure and co-evaporated twice with anhydrous toluene. The residue was taken in methanol (20 ml) and treated with 7N ammonia in methanol (2 ml) and stirred at room temperature. After completion, the reaction mixture was concentrated under reduced pressure and purified by column chromatography to provide the final desired product.

### Example 89. Synthesis of 4′-fluoro-2-thiouridine

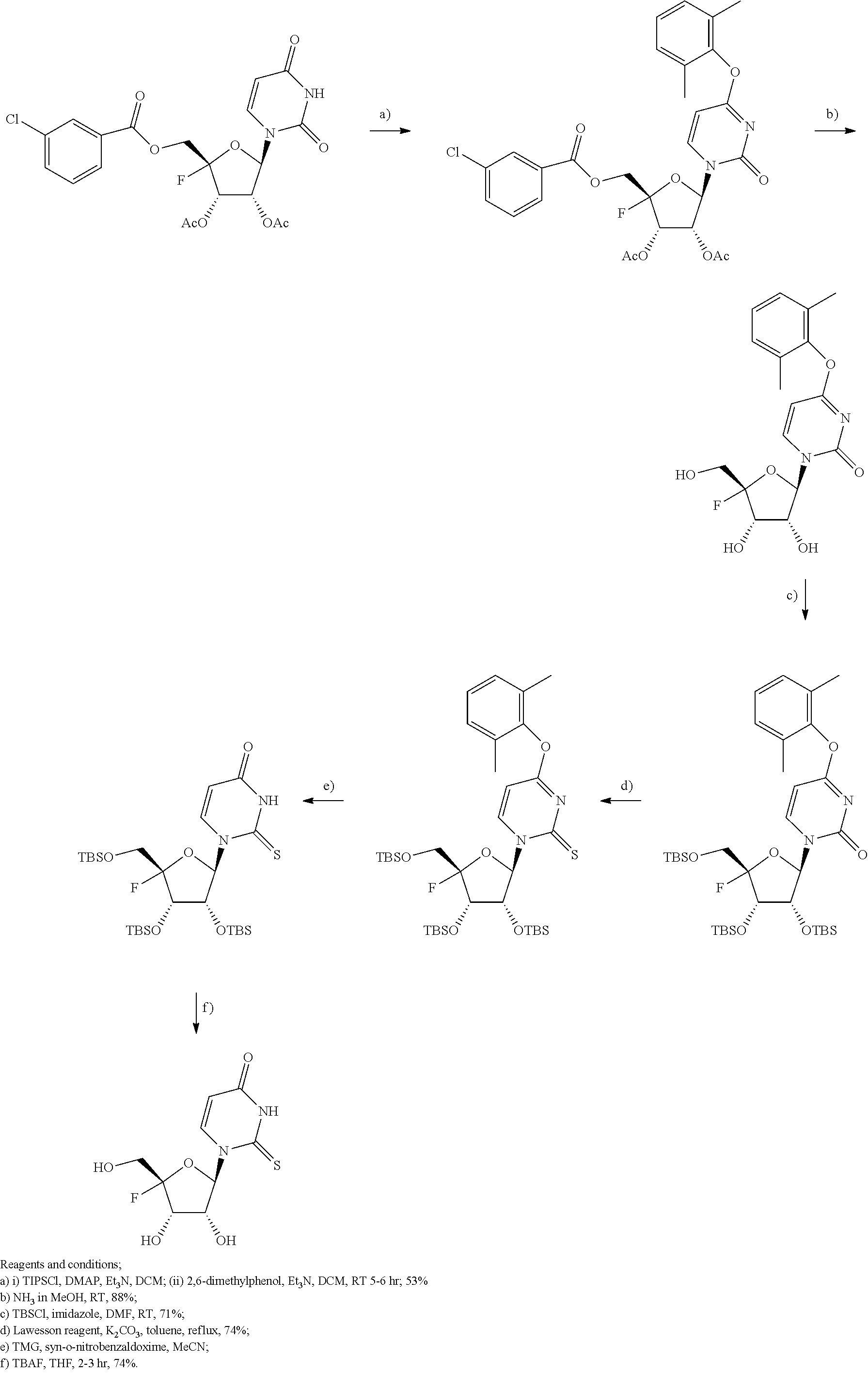

### Preparation of 4-O-(2,6-dimethylphenyl)-2′,3′-di-O-acetyl-5′-O-(4-chlorobenzoyl)-4′-fluorouridine

2′,3′-di-O-acetyl-5′-O-(4-chlorobenzoyl)-4′-fluorouridine (1 gm, 2 mmol) was dissolved in anhydrous dichloromethane (30 ml) in 100 ml RBF. Et₃N (542 μL, 3.75 mmol), 2,4,6-triisopropylbenzensulfonyl chloride (690 mg, 2.26 mmol), and 4-(dimethylamino)pyridine (62 mg, 0.5 mmol) were added at 0° C. under an inert atmosphere with continued stirring at room temperature. After completion of the reaction, 2,6-dimethylphenol (300 mg, 2.45 mmol), Et₃N (3.45 mL, 25 mmol), and 1,4-diazabicyclo [2,2,2]octane (23 mg, 0.2 mmol) were added at 0° C. under an inert atmosphere with continued stirring at room temperature for 3-4 hr. The reaction mixture was diluted with dichloromethane (30 ml) and washed once with saturated NaHCO₃ (aqueous) and twice with brine. The combined organic extracts were dried over Na₂SO₄, filtered, and concentrated under reduced pressure. The residue was purified by column chromatography. Product was obtained as colorless solid (yield 53%).

### Preparation of 4-O-(2,6-Dimethylphenyl)-4′-fluorouridine

To a solution of 4-O-(2,6-Dimethylphenyl)-2′,3′-di-O-acetyl-5′-O-(4-chlorobenzoyl)-4′-fluorouridine (600 mg) in anhydrous methanol (6 ml) in 25 ml RBF, 1 ml of 7N ammonia in methanol was added and stirred at room temperature for 8 hr. After completion, the reaction mixture was concentrated under reduced pressure and the crude product was obtained as colorless solid (yield 88%).

### Preparation of 4-O-(2,6-dimethylphenyl)-2′,3′,5′-tri-O-(t-butyldimethylsilyl)-4′-fluorouridine

To a solution of 4-O-(2,6-dimethylphenyl)-4′-fluorouridine (720 mg) in anhydrous DMF (10 ml) in a 50 ml RBF, tert-butyldimethylsilyl chloride (1185 mg, 7.8 mmol) and imidazole (670 mg, 9.8 mmol) were added at 0° C. under an inert atmosphere with continued stirring at room temperature for 12 hr. After completion the reaction mixture was concentrated under reduced pressure and the crude product was taken up in DCM and washed with saturated aq.NaHCO₃ and with brine. The combined organic extracts were dried over Na₂SO4, filtered, and concentrated under reduced pressure. The residue was purified by column chromatography to get colorless foam (yield 71%)

### Preparation of 4-O-(2,6-dimethylphenyl)-2′,3′,5′-tri-O-(t-butyldimethylsilyl)-4′-fluoro-2-thiouridine

To a solution of 4-O-(2,6-dimethylphenyl)-2′,3′,5′-tri-O-(t-butyldimethylsilyl)-4′-fluorouridine (750 mg, 1 mmol) in anhydrous toluene (20 ml), Lawesson's reagent (freshly purchased) (590 mg, 1.5 mmol) and potassium carbonate (29 mg, 0.2 mmol) were added and the reaction mixture was refluxed for 8 hr. After completion, the reaction mixture was concentrated under reduced pressure and the crude product was purified by column chromatography. Product was obtained as colorless foam (yield 74%).

### Preparation of 2′,3′,5′-tri-O-(t-butyldimethylsilyl)-4′-fluoro-2-thiouridine

To a solution of 4-O-(2,6-dimethylphenyl)-2′,3′,5′-tri-O-(t-butyldimethylsilyl)-4′-fluoro-2-thiouridine (500 mg, 0.68 mmol) in acetonitrile (10 ml), 1,1,3,3-tetramethylguanidine (260 μL, 2 mmol) and syn-o-nitrobenzaldoxime (343 mg, 2 mmol) were added and stirred at room temperature for 5 hr. After completion, the mixture was concentrated under reduced pressure. The crude product was dissolved in dichloromethane and washed with saturated aq. NaHCO₃ and with brine. The combined organic layers were dried over Na₂SO₄, filtered, and concentrated under reduced pressure. The residue was purified by chromatography. Product was obtained as colorless foam (yield 67%).

### Preparation of 4′-fluoro-2-thiouridine

To a solution of 2′,3′,5′-tri-O-(t-butyldimethylsilyl)-4′-fluoro-2-thiouridine (270 mg, 0.43 mmol) in anhydrous tetrahydrofuran (5 ml), 1M solution of tetrabutylammonium fluoride (2 ml) was added and stirred at room temperature for 5 hr. After completion, the reaction mixture was concentrated under reduced pressure and the crude product was purified by silica gel column chromatography. Product was obtained as off white solid (yield 74%). 1H NMR 400 MHz, CD₃OD, δ 8.11 (1H, d, J=8 Hz), 6.84 (1H, s), 5.94 (1H, d, J=8 Hz), 4.26 (2H, m), 3.78 (2H, m); 13C NMR 100 MHz δ 176.49, 159.90, 140.78, 119.46, 117.16, 107.34, 95.08, 72.83, 68.59, 59.80; 19F NMR 376 MHz 6-122.77, (1F, d, J=18.8 Hz); LCMS: [M+1]+279.0.

### Example 90. Protocol for Determining Plasma Stability

Test article was incubated in triplicate at 1.00 μM in pooled mixed gender human plasma (BioIVT, K₂EDTA), in pooled male CD-1 mouse plasma (BioIVT, K₂EDTA), in pooled male Sprague-Dawley rat plasma (BioIVT, lithium heparin). Incubations were performed in 13×100 mm glass culture tubes. Samples were placed in a water bath shaker set at 37° C. and shaken at 150 rpm. Procaine, Benfluorex or Enalapril (1 μM, each) were run in parallel as a positive controls for human, mouse or rat plasma activity, respectively. Aliquots of 100 μL were taken at the following time-points: 0, 5, 15, 30, 60, and 120 minutes. These aliquots were mixed with 400 μL of 100% acetonitrile in 1.7-mL conical polypropylene microcentrifuge tubes. Samples were vortexed for about 10 seconds and then clarified by centrifugation (2 minutes at 15,000 g). Supernatants were analyzed by LC-MS/MS.

HPLC separation was performed on an Agilent 1200 system (Agilent Technologies, Santa Clara, CA, USA) equipped with a column oven, UV lamp, and binary pump. A Thermo Hypercarb PGC (150×4.6 mm, 5 μm) column (ThermoFisher, Waltham, MA USA) was used for the separation. Mobile Phase A consisted of 100 mM Ammonium Bicarbonate buffer in HPLC grade Water (pH 10) and Mobile phase B consisted of neat acetonitrile. A gradient 0-85% of B was run for 3 minutes followed by 0% B for 4 minutes was used for the separation. Mass Spectrometry analysis was performed on a Triple Quad 5500 Mass Spectrometer (AB Sciex, Farmingham, MA, USA) using Negative Mode Electrospray Ionization (ESI) in Multiple Reaction Monitoring (MRM) Mode. Data analysis was performed using Analyst Software (AB Sciex, Farmingham, MA, USA). Analyte concentrations were calculated based on standard curve. Half-lives (t_{1/2}) were calculated by plotting the natural logarithm of the analyte concentration vs. time and obtaining the slope of the line. Assuming first-order kinetics, the elimination rate constant, k, is the negative (−) of the slope of the plot (ln [μM]vs. time). Half-life (t_{1/2}) (min)=−0.693/(slope).

### Example 91. Protocol for Determining Liver Microsome Stability

Test article was incubated in triplicate at 1.00 μM in 100 mM phosphate buffer (pH 7.4), Phase I cofactors (NADPH Regenerating System) and 0.5 mg (total protein) from pooled gender human liver microsomes (BioIVT), pooled male CD-1 mouse liver microsomes (XenoTech) or pooled male Sprague-Dawley rat liver microsomes (BioIVT). Incubations were performed in 13×100 mm glass culture tubes. Samples were placed in a water bath shaker set at 37° C. and shaken at 150 rpm. Verapamil (1 μM) was run in parallel as a positive control.

HPLC separation was performed on an Agilent 1200 system (Agilent Technologies, Santa Clara, CA, USA) equipped with a column oven, UV lamp, and binary pump. A Thermo Hypercarb PGC (150×4.6 mm, 5 μm) column (ThermoFisher, Waltham, MA USA) was used for the separation. Mobile Phase A consisted of 100 mM Ammonium Bicarbonate buffer in HPLC grade Water (pH 10) and Mobile phase B consisted of neat acetonitrile. A gradient 0-85% of B was run for 3 minutes followed by 0% B for 4 minutes were used for the separation. Mass Spectrometry analysis was performed on a Triple Quad 5500 Mass Spectrometer (AB Sciex, Farmingham, MA, USA) using Negative Mode Electrospray Ionization (ESI) in Multiple Reaction Monitoring (MRM) Mode. Data analysis was performed using Analyst Software (AB Sciex, Farmingham, MA, USA).

Analyte concentrations were calculated based on Standard curve. Half-lives (t_{1/2}) were calculated by plotting the natural logarithm of the analyte concentration vs. time and obtaining the slope of the line. Assuming first-order kinetics, the elimination rate constant, k, is the negative (−) of the slope of the plot (ln [μM]vs. time). Half-life (t_{1/2}) (min)=−0.693/(slope).

### Example 92. Protocol for Determining pH Stability

Test article in methanol, water, 0.1N HCl, PBS or pH9 buffer were placed in the HPLC autosampler set at 25° C. or 4° C. Samples were injected on the LC-MS/MS at times: 0, 1, 2, 3, 4, 6 and 24 hours. HPLC separation was performed on an Agilent 1200 system (Agilent Technologies, Santa Clara, CA, USA) equipped with a column oven, UV lamp, and binary pump. A Thermo Hypercarb PGC (100×4.6 mm, 5 μm) column (ThermoFisher, Waltham, MA USA) was used for the separation. Mobile Phase A consisted of 25 mM ammonium bicarbonate buffer in HPLC grade water (pH 9.4) and Mobile phase B consisted of neat acetonitrile. Initial mobile phase conditions of 5% B were held for a minute. A gradient 5-60% of B was run for next 7 minutes, followed by re-equilibration of the column, was used. Mass Spectrometry analysis was performed on a QTRAP 5500 Mass Spectrometer (AB Sciex, Framingham, MA, USA) using Negative Mode Electrospray Ionization (ESI) in Multiple Reaction Monitoring (MRM) Mode and UV at 260 nm. Data analysis was performed using Analyst Software (AB Sciex, Framingham, MA, USA). Stability was determined by the % UV peak area change from the time-zero samples.

### Example 93. Stability of EIDD-02749 in Solvents and Buffers

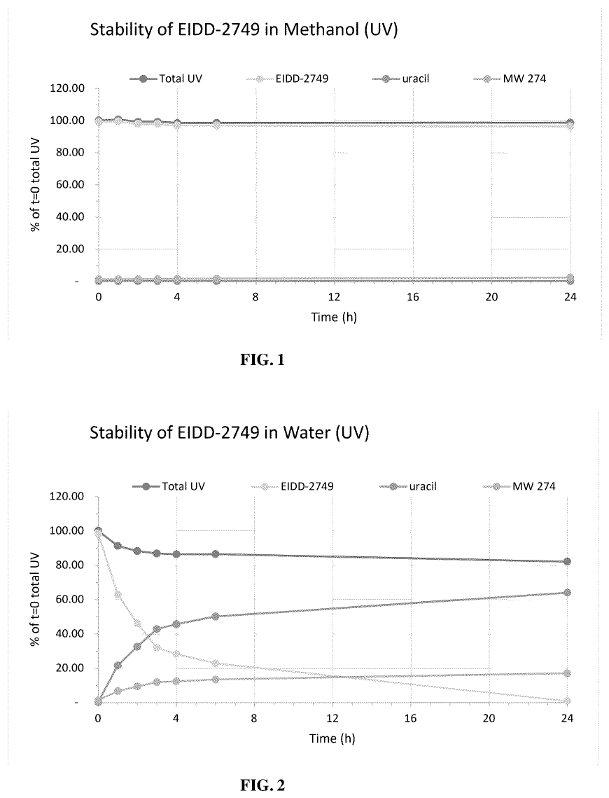

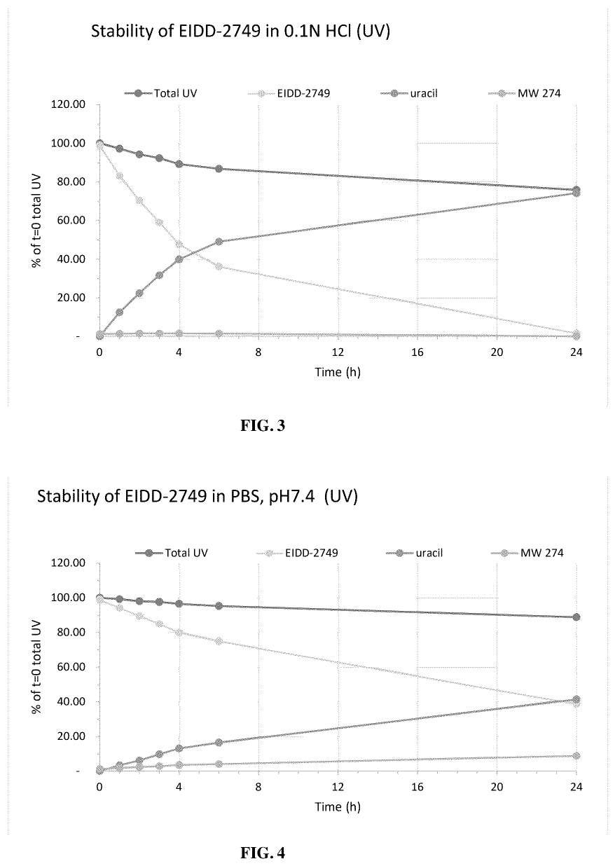

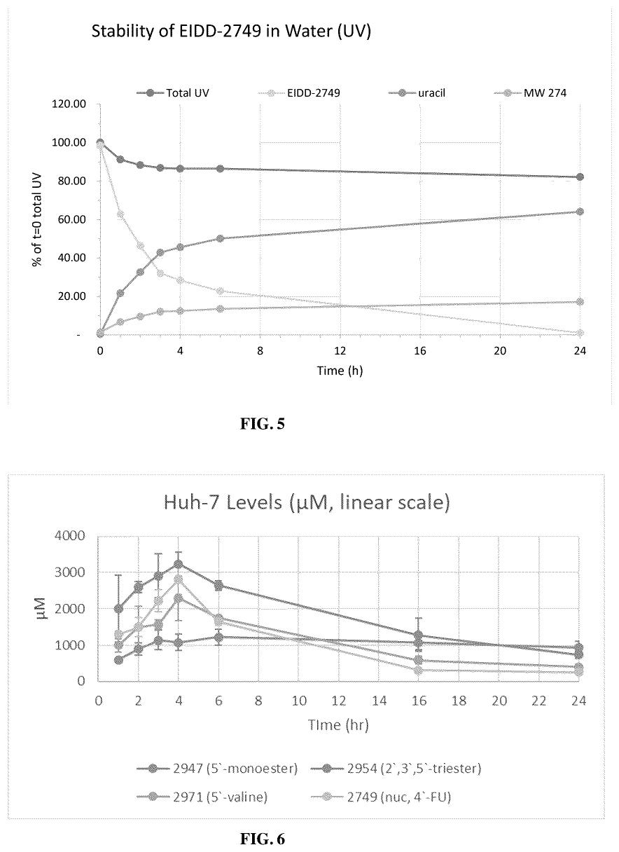

The stability of EIDD-02749 in solvents and acidic, neutral, and basic buffers is shown in FIGS. 1-5.

### Example 94. Stability of EIDD-02749 Prodrugs

| | | | half-life | max |
|---|---|---|---|---|
| species | matrix | drug (1.00 μM, each) | (min) | [2749], μM |
| human | plasma | EIDD-2749 (nuc) | 26 | NA |
| | | EIDD-2838 (cycloSal) | 12 | 0.0521 |
| | | EIDD-2947 (mono-ester) | 2 | 0.653 |
| | | EIDD-2954 (tri-ester) | 2 | 0.0390 |
| | | EIDD-2971 (valine) | 81 | 0.0152 |
| | liver | EIDD-2749 (nuc) | 22 | NA |
| | microsomes | | | |
| | | EIDD-2838 (cycloSal) | 7 | 0.130 |
| | | EIDD-2947 (mono-ester) | 44 | 0.209 |
| | | EIDD-2954 (tri-ester) | <1 | 0.342 |
| | | EIDD-2971 (valine) | 38 | 0.179 |
| mouse | plasma | EIDD-2749 (nuc) | 37 | NA |
| | | EIDD-2838 (cycloSal) | 1 | 0.0216 |
| | | EIDD-2947 (mono-ester) | 1 | 1.18 |
| | | EIDD-2954 (tri-ester) | 1 | 0.716 |
| | | EIDD-2971 (valine) | 59 | 0.267 |
| | liver | EIDD-2749 (nuc) | 27 | NA |
| | microsomes | | | |
| | | EIDD-2838 (cycloSal) | 7 | 0.0347 |
| | | EIDD-2947 (mono-ester) | 1 | 0.602 |
| | | EIDD-2954 (tri-ester) | <1 | 0.256 |
| | | EIDD-2971 (valine) | 11 | 0.305 |
| rat | plasma | EIDD-2749 (nuc) | 22 | NA |
| | | EIDD-2838 (cycloSal) | <1 | 0.0341 |
| | | EIDD-2947 (mono-ester) | 9 | 0.518 |
| | | EIDD-2954 (tri-ester) | 1 | 0.142 |
| | | EIDD-2971 (valine) | 50 | 0.155 |
| | liver | EIDD-2749 (nuc) | 25 | NA |
| | microsomes | | | |
| | | EIDD-2838 (cycloSal) | 8 | 0.218 |
| | | EIDD-2947 (mono-ester) | 10 | 0.423 |
| | | EIDD-2954 (tri-ester) | <1 | 0.149 |
| | | EIDD-2971 (valine) | 33 | 0.203 |

### Example 95. EIDD-02991 Concentrations in Huh-7 Cells

EIDD-02991 concentrations in Huh-7 cells incubated with EIDD-02749, EIDD-02947, EIDD-02954, or EIDD-02971 are shown in FIG. 6.

### Example 96. EIDD-02991 Concentrations in Vero Cells

EIDD-02991 concentrations in Vero cells incubated with EIDD-02749, EIDD-02947, EIDD-02954, or EIDD-02971 are shown in FIG. 7.

### Example 83. Mouse PK protocol

Female ICR (CD-1) mice (from Envigo) between the ages of 7 to 8 weeks were acclimated to their environment for at least three days prior to dosing. Mice were weighed at least once before dosing to determine the dosing volume.

Test article was dissolved in sterile saline at 1 mg/mL for IP dosing. For oral dosing, test article was resuspended in 10 mM trisodium citrate/0.5% Tween 80/Water. For IP dosing mice were dosed with a 10 mL/kg dose volume and mice dosed PO were dosed with a 10 mL/kg dose volume.

Blood samples collected from mice dosed by oral gavage were collected pre-dose, 0.25, 0.50, 1, 2, 3, 4, 8, and 24 hours post-dose. Blood samples collected from mice dosed by intraperitoneal injection were collected pre-dose, 0.08, 0.25, 0.50, 1, 2, 3, 4, and 8 hours post-dose. Blood samples were collected by reto-orbital bleeding under isoflurane anesthesia into lithium-heparin microtainer tubes, centrifuged at 2000×g for 10 min at 5^{C}, and the plasmas were transferred into fresh tubes and stored at −80° C. before processing for quantitation by LC-MS/MS. 50 μL aliquots of mouse plasma were extracted with 950 μL of acetonitrile that included EIDD-2216 as an Internal Standard. Samples were clarified by centrifugation at 20,000×g at 4° C. for 10 min. The clarified supernatants were transferred to HPLC vials for analysis.

Samples were maintained at 4° C. in a Leap Pal Autosampler (CTC Analytics AG, Zwingen, Switzerland). HPLC separation was performed on an Agilent 1200 system (Agilent Technologies, Santa Clara, CA, USA) equipped with a column oven, UV lamp, and binary pump. An Agilent SB-Phenyl (150×4.6 mm, 5 μm) column (Agilent technologies, Santa Clara, CA, USA) was used for the separation. Mobile Phase A consisted of 100 mM Ammonium Formate buffer in HPLC grade Water and Mobile phase B consisted of pure acetonitrile. An initial 1 minute isocratic step was used at 5% Mobile Phase B followed by a 1.5 minute gradient to 100% Mobile Phase B, which was held for 1.5 minutes before returning to starting conditions for 1.5 minutes. Mass Spectrometry analysis was performed on an QTRAP 5500 Mass Spectrometer (AB Sciex, Farmingham, MA, USA) using Negative Mode Electrospray Ionization (ESI) in Multiple Reaction Monitoring (MRM) Mode. Data analysis was performed using Analyst Software (AB Sciex, Farmingham, MA, USA).

PK parameters are calculated using the Phoenix WinNonLin 6.4 (Build 6.4.0.768) Non-compartmental analysis tool (Certara, Princeton, NJ, USA). Bioavailability is calculated by comparing the exposure (AUCinf) after oral dosing with the exposure after intraperitoneal dosing.

### Example 84. EIDD-02749 Mouse PK Results

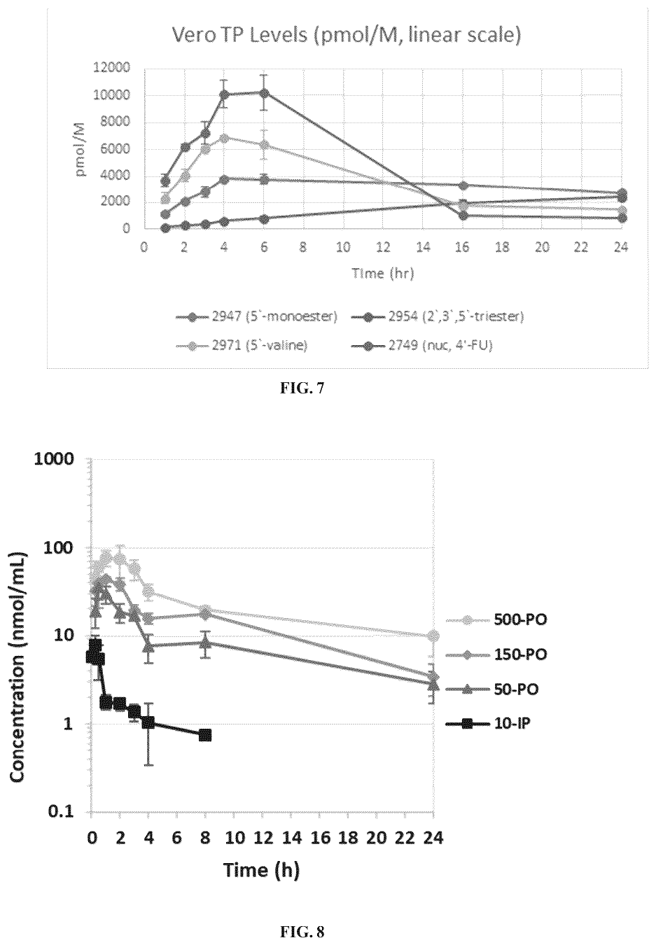

Graphs for EIDD-02749 CD-1 mouse PK are shown in FIG. 8. The PK parameters are shown in the table below.

| | | | | | Cₘₐₓ/Dose | AUC-INF/Dose | | |
|---|---|---|---|---|---|---|---|---|
| Dose | t [symbol] | C [symbol] | C [symbol] | AUC-INF | (kg*nmol/ | (h*kg*nmol/ | t_{1/2} | |
| (mg/kg) | (h) | (nmol/mL) | (nmol/mL) | (h*nmol/mL) | mL/mmol) | mL/mmol) | (h) | Bioavailability* |
| 10-IP | 0.25 | 7.7 ± 2.3 | 0.75 ± 0.1 | 19.6 | 202.1 | 514.5 | 6.3 | N/A |
| 50-PO | 0.5 | 35.2 ± 9.7 | 2.8 ± 1.1 | 232.3 | 184.3 | 1215.0 | 8.3 | 236% |
| 150-PO | 1 | 43.6 ± 2.8 | 3.4 ± 1.3 | 392.3 | 76.2 | 685.8 | 8.5 | 133% |
| 500-PO | 1 | 76.0 ± 15.3 | 9.8 ± 4.2 | 755.2 | 39.9 | 395.0 | 12.9 | 77.0% |

[symbol]

indicates data missing or illegible when filed

### Example 85. Mouse Tolerability Protocol

AG129 mice between the ages of 6 to 10 weeks were acclimated to their environment for at least three days prior to dosing. Mice were weighed daily as well as monitored for morbidity and mortality daily. For oral dosing, test article was resuspended in 10 mM trisodium citrate/0.5% Tween 80/Water. Mice dosed PO were dosed with a 10 mL/kg dose volume. Mice were dosed PO at 10, 30, and 100 mg/kg QD for 10 days.

### Example 86. Tolerability of EIDD-02749 in AG129 Mice

Results for tolerability of EIDD-02749 in AG129 mice are shown in FIG. 9.

### Example 87. MERS Activity for EIDD-02749

| Virus | Strain | Cell Line | EC₅₀ (μM) | CC₅₀ (μM) |
|---|---|---|---|---|
| MERS | EMC | Vero 76 | 5.7 | >380 |

### Example 88. Measles Activity for EIDD-02749

| Virus | Strain | Cell Line | EC₅₀ (μM) | CC₅₀ (μM) |
|---|---|---|---|---|
| Measles | CC | Vero 76 | 1.06 | >380 |

### Example 89. Picornaviridae Activity for EIDD-02749

| | | | EC₅₀ | EC₉₀ | CC₅₀ |
|---|---|---|---|---|---|
| Virus | Strain | Cell Line | (μM) | (μM) | (μM) |
| Coxsackie | HA 201933 | Vero 76 | 1.63 | 7.22 | 372 |
| ENTV-68 | US/KY/14-18953 | RD | 0.65 | 1.06 | 228 |
| Poliovirus | Mahoney | Vero 76 | 2.01 | 3.8 | >380 |

### Example 90. Togaviridae Activity for EIDD-02749

| | | | EC₅₀ | EC₉₀ | CC₅₀ |
|---|---|---|---|---|---|
| Virus | Strain | Cell Line | (μM) | (μM) | (μM) |
| EEEV | Fl39-939 | Vero 76 | 0.65 | 1.79 | >380 |
| Mayaro | BEAR 505411 | Vero 76 | 2.81 | 9.5 | >380 |

### Example 91. Hantaviridae Activity for EIDD-02749

| Virus | Cell Line | EC₅₀ (μM) | CC₅₀ (μM) |
|---|---|---|---|
| Andes | Vero | 0.11 | >100 |
| Andes | Vero | 0.035 | >100 |
| Andes | Vero | 0.028 | >100 |

### Example 92. Arenaviridae Activity for EIDD-02749

| | | | EC₅₀ | EC₉₀ | CC₅₀ |
|---|---|---|---|---|---|
| Virus | Strain | Cell Line | (μM) | (μM) | (μM) |
| Tacribe | TRVL 11573 | Vero | <0.12 | <0.12 | >380 |
| Tacribe | TRVL 11573 | Vero | <0.12 | <0.12 | >380 |
| Tacribe | TRVL 11573 | Vero | 0.13 | 0.33 | >380 |
| Pichinde | An 4763 | Vero | 3.5 | 0.32 | >380 |
| Pichinde | An 4763 | Vero | 1.9 | 0.36 | >380 |
| Junin | Candid #1 | Vero | 0.33 | <0.12 | >380 |
| Junin | Candid #1 | Vero | 0.35 | <0.12 | >380 |
| Junin | Romero | HeLa | 0.02 | — | 77 |
| Junin | Romero | Huh-7 | 0.25 | — | >100 |
| Junin | Romero | Vero E6 | 0.08 | — | >100 |
| Lassa | Josiah | HeLa | 0.45 | — | 77 |
| Lassa | Josiah | Huh-7 | 0.9 | — | >100 |
| Lassa | Josiah | Vero E6 | 0.36 | — | >100 |
| Machupo | Carvallo | HeLa | 0.07 | — | 77 |
| Machupo | Carvallo | Huh-7 | 0.31 | — | >100 |
| Machupo | Carvallo | Vero E6 | 1.49 | — | >100 |
| Lujo | Zambia 2008 | HeLa | 0.07 | — | 77 |
| Lujo | Zambia 2008 | Huh-7 | 1.32 | — | >100 |
| Lujo | Zambia 2008 | Vero E6 | 0.83 | — | >100 |

### Example 93. Cytotoxicity for EIDD-02749

| Cell Line | CEM | Huh7 | HG23 | HepG2/Gal* | BxPC-3 | A204 | RD | HBTECs | BEAS-28 | BEAS-28/Gal* | A549 |
|---|---|---|---|---|---|---|---|---|---|---|---|
| Species | Human | Human | Human | Human | Human | Human | Human | Human | Human | Human | Human |
| Cell Type | Lymphoid | Liver | [symbol] | [symbol] | Pancreas | Muscle | Muscle | Respiratory | Lung | Lung | Lung |
| | | | | | | | | Tract | | | |
| CC [symbol] (μM) | 107 | >400 | 100 | >400 | >400 | >400 | >380 | >500 | >500 | 250 | >400 |
| | | (n = 3) | | | | | | | | | |

| Cell Line | Hep-2 | Hela | IEC-6 | H9c2 | RAW 264.7 | Vero | MA-104 | MDCK | MDCK/Gal* | BHK-T7 |
|---|---|---|---|---|---|---|---|---|---|---|
| Species | Human | Human | Rat | Rat | Mouse | Green | Monkey | Canine | Canine | Hamster |
| | | | | | | Monkey | | | | |
| Cell Type | Larynx | Cervix | Small | Heart | Macrophage | Kidney | Kidney | Kidney | Kidney | Kidney |
| | | | intestine | | | | | | | |
| CC [symbol] (μM) | [symbol] | [symbol] ± 32 | >400 | >400 | >100 | >380 | >380 | >380 | 444 | >500 |
| | | (n = 2) | | | | (n = 42) | (n = 2) | (n = 6) | | |

[symbol]

indicates data missing or illegible when filed

### Example 94. Mouse PK Parameters for EIDD-02749

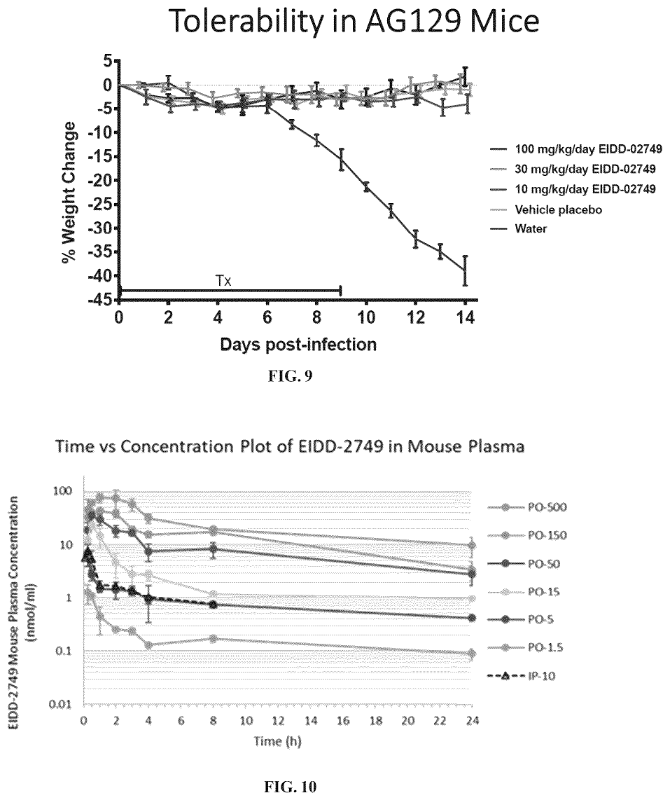

Graphs for EIDD-02749 CD-1 mouse PK are shown in FIG. 10. The PK parameters are shown in the table below.

| Dosing | Dose | Tmax | Cmax | T_{1/2} | Auc (0-∞) |
|---|---|---|---|---|---|
| Route | (mg/kg) | (h) | (nmol/mL) | (h) | (h*nmol/mL) |
| IP | 10 | 0.25 | 7.7 | 6.3 | 19.6 |
| PO | 1.5 | 0.25 | 1.2 | 17.9 | 6.58 |
| PO | 5 | 0.25 | 7.1 | 15.2 | 28.6 |
| PO | 15 | 0.5 | 23.3 | 20.3 | 86.9 |
| PO | 50 | 0.5 | 35.2 | 8.28 | 232.3 |
| PO | 150 | 1 | 43.6 | 8.46 | 392.3 |
| PO | 500 | 1 | 76 | 12.9 | 755.2 |

### Example 95. Ferret PK Parameters for EIDD-02749

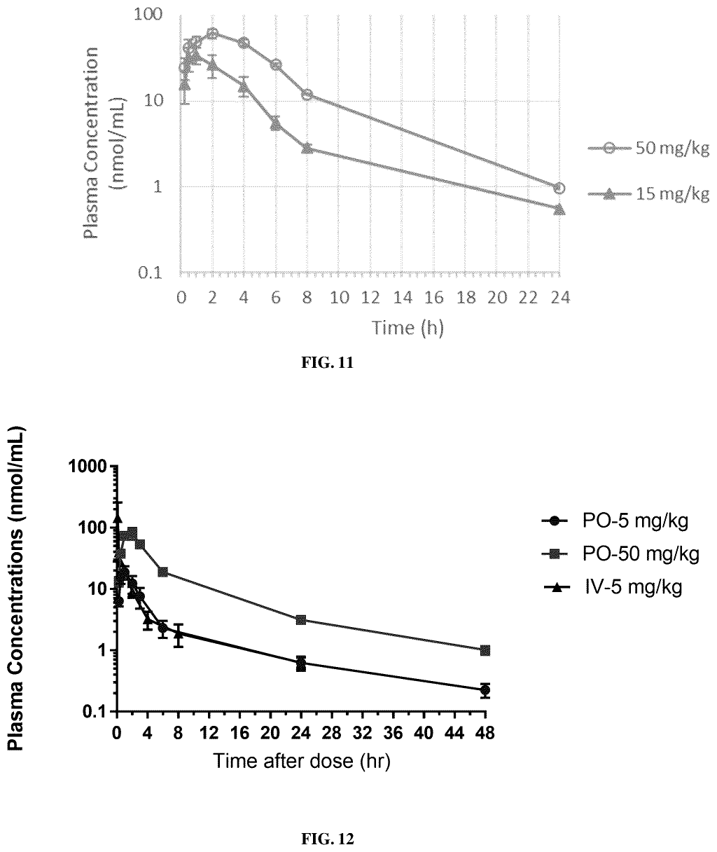

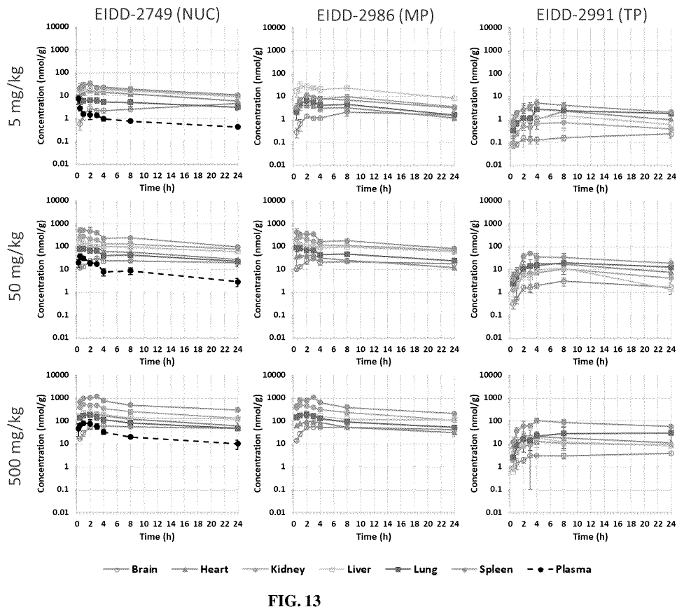

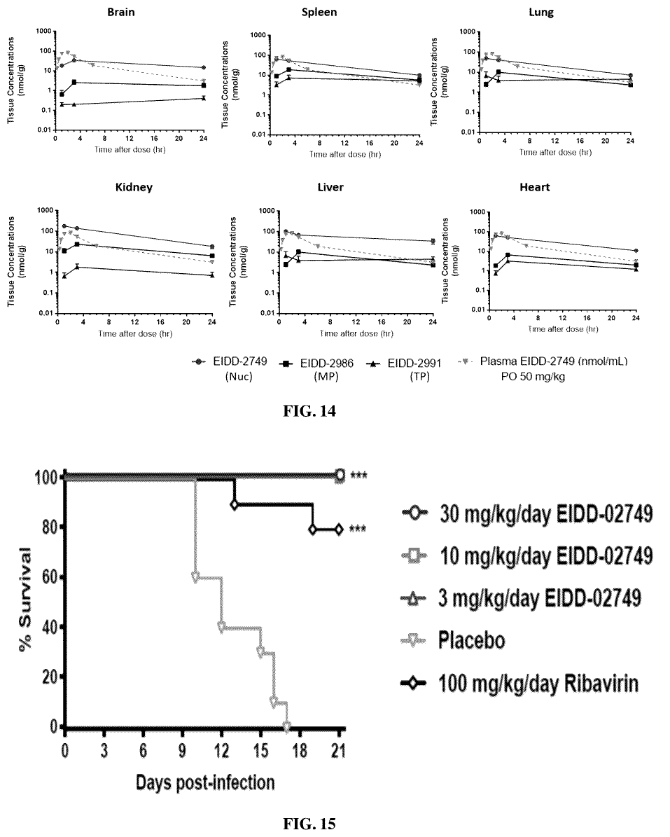

Graphs for EIDD-02749 ferret PK are shown in FIG. 11. The PK parameters are shown in the table below.

| | | | | Cₘₐₓ/Dose | AUC-INF/Dose | |
|---|---|---|---|---|---|---|
| Dose | tₘₐₓ | Cₘₐₓ | AUC-INF | (kg*nmol/ | (h*kg*nmol/ | t_{1/2} |
| (mg/kg) | (h) | (nmol/mL) | (h*nmol/ml) | mL/mmol) | mL/mmol) | (h) |
| 15 | 1.0 ± 0.0 | 34.8 ± 6.5 | 154.0 ± 27.6 | 608.2 ± 114.0 | 2691.2 ± 483.7 | 5.97 ± 0.35 |
| 50 | 2.3 ± 1.5 | 63.3 ± 10.9 | 413.1 ± 78.1 | 332.0 ± 57.5 | 2166.1 ± 409.8 | 4.3 ± 0.80 |

### Example 96. Guinea Pig PK Parameters for EIDD-02749

Graphs for EIDD-02749 Guinea pig PK are shown in FIG. 12. The PK parameters are shown in the table below.

| Dose | tₘₐₓ | Cₘₐₓ | AUC_{0->∞} | Cₘₐₓ/Dose | AUC_{0->∞}/Dose | V_{D} | CL | t_{1/2} |
|---|---|---|---|---|---|---|---|---|
| (mg/kg) | (h) | (nmol/mL) | (h · nmol/mL) | (kg · nmol/mL · mmol) | (h · kg · nmol/mL · mmol) | (L/kg) | (L/h · kg) | (h) |
| 5 IV | 0.083 | 143.5 ± 115.4 | 107.5 ± 4.5 | 7525.0 ± 6050.9 | 5639.4 ± 234.2 | 2.4. ± 0.4 | 0.18 ± 0.01 | 9.3 ± 1.3 |
| 5 PO | 1.3 ± 0.5 | 20.1 ± 2.3 | 92.9 ± 16.1 | 1054.5 ± 118.8 | 4869.4 ± 841.9 | 13.0 ± 1.6 | 1.3 ± 0.5 | 20.1 ± 2.3 |
| 50 PO | 1.8 ± 0.5 | 86.4 ± 9.8 | 557.5 ± 39.9 | 453.0 ± 51.2 | 2923.1 ± 209.6 | 14.9 ± 1.9 | 1.8 ± 0.5 | 86.4 ± 9.8 |

### Example 97. Prophylactic Efficacy of EIDD-02749 in a Mouse Model of Tacaribe Infection

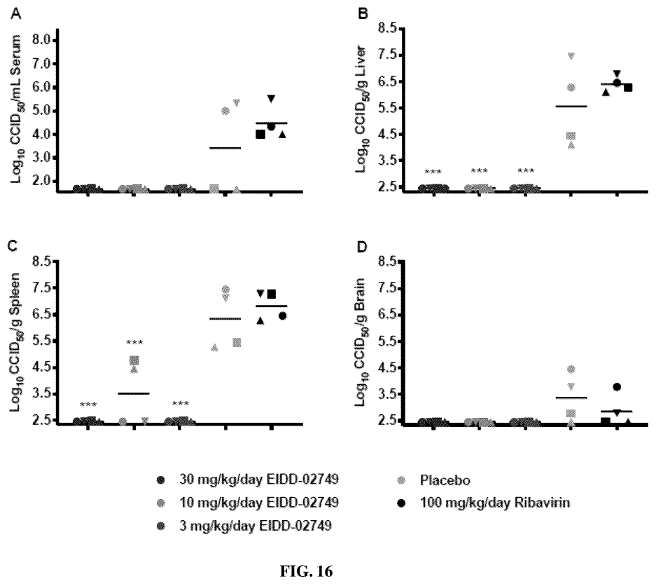

AG129 mice, inoculated with 500 CCID₅₀ TCRV (strain TRVL 11573) i.p., received 3, 10, or 30 mg/kg/d EIDD-02749 PO for 14 days starting 2 hours before infection. Survival curves for the study are shown in FIG. 15 and viral titers measured on Day 9 are shown in FIG. 16.

### Example 98. Therapeutic Efficacy of EIDD-02749 in a Mouse Model of Tacaribe Infection

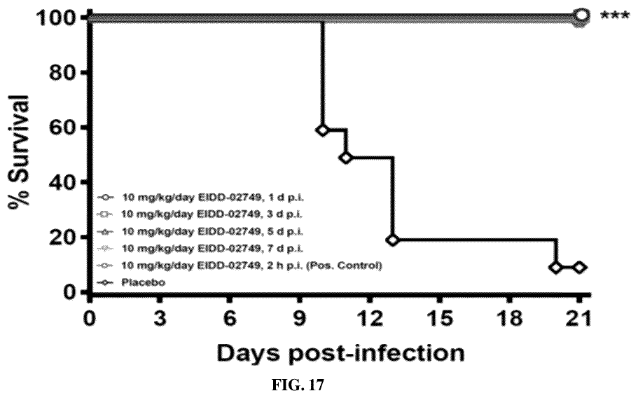

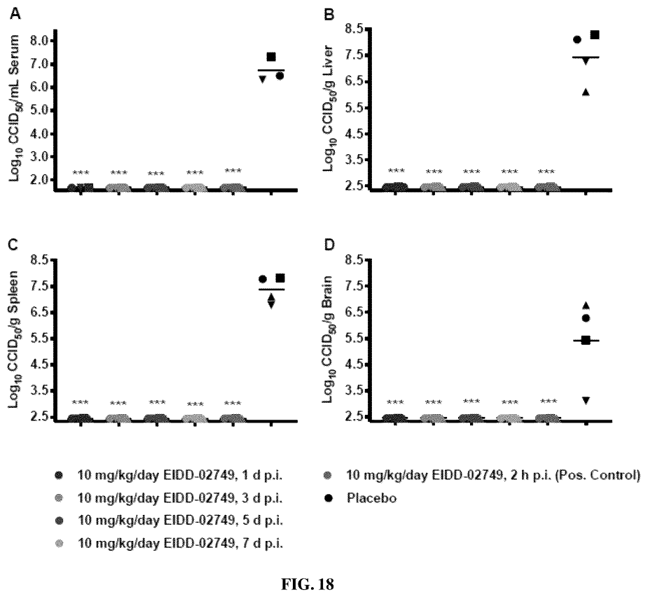

AG129 mice, inoculated with 500 CCID₅₀ TCRV (strain TRVL 11573) i.p., received 10 mg/kg/d EIDD-02749 PO. Treatment started 2 hours, 1 Day, and 3 Days post infection and continued for 14 days, 5 Days post infection and continued for 12 days, and 7 Days post infection and continued for 10 days. Survival curves for the study are shown in FIG. 17 and viral titers measured on Day 9 are shown in FIG. 18.

### Example 99: SARS Assay

Calu: At 48 hrs prior to infection, Calu3 2B4 cells were plated in a 96-well black walled clear bottom plate at 5×10⁴ cells/well. A 10 mM stock of EIDD-2749 was serially diluted in 100% DMSO in 3-fold increments to obtain a ten-point dilution series. MERS-nLUC was diluted in DMEM 10% FBS, and 1% Antibiotic-Antimycotic to achieve a multiplicity of infection (MOI) of 0.08. Cells were infected and treated with EIDD-2749 in triplicate per drug dilution for 1 hr after which, viral inoculum was aspirated, cultures were rinsed once and fresh medium containing drug or vehicle was added. At 48 hrs post infection, nanoluciferase expression as a surrogate for virus replication was quantitated on a Spectramax (Molecular Devices) plate reader according to the manufacturer's instructions (Promega, NanoGlo). For our 100% inhibition control, diluted MERS-nLUC was exposed to short-wave UV light (UVP, LLC) for 6 minutes to inhibit the ability of the virus to replicate. For our 0% inhibition control, cells were infected in the presence of vehicle only. DMSO was kept constant in all conditions at 0.05%. Values from triplicate wells per condition were averaged and compared to controls to generate a percent inhibition value for each drug dilution. The IC₅₀ value was defined as the concentration at which there was a 50% decrease in luciferase expression. Data was analyzed using GraphPad Prism 8.0 (La Jolla, CA). The IC₅₀ values were calculated by non-linear regression analysis using the dose-response (variable slope) equation (four parameter logistic equation): Y=Bottom+(Top−Bottom)/(1+10\hat{ }((LogIC50−X)*HillSlope)). To measure cell viability to determine if there was any NHC induced cytotoxicity, Calu3 2B4 cells were plated and treated with EIDD-2749 only as described above. Cells were exposed to the same ten-point dilution series created for the in vitro efficacy studies. As above, 0.05% DMSO-treated cells served as our 0% cytotoxicity control. Wells without cells served as our 100% cytotoxic positive control. After 48 hr, cell viability was measured on a Spectramax (Molecular Devices) via Cell-Titer Glo Assay (Promega) according to the manufacturer's protocol. Similar data was obtained in three independent experiments.

### Example 100: EIDD-2749 Coronavirus Activity Including COVID-19 (SARS-2) Activity

| | | | EC₅₀ | EC₉₀ | CC₅₀ |
|---|---|---|---|---|---|
| Virus | Strain | Cell Line | (μM)^{a} | (μM)^{b} | (μM) |
| MERS | — | Vero 76 | 5.7 | 9.5 | >380 |
| HCoV-alpha | 229E | Huh-7 | 1.67 | 9.5 | >380 |
| HCoV-beta | OC43 | RD | 1.06 | 2.85 | >380 |
| SARS | Urbani | Vero 76 | 14.1 | — | >380 |
| SARS-CoV-2 | USA_WA1/2020 | Vero 76 | 2.3 | 1.52 | >380 |
| SARS-CoV-2 | USA_WA1/2020 | Calu3 | 1.9 | — | >50 |

1.-396. (canceled)

397. A method of treating a viral infection in a subject in need thereof, comprising administering to the subject a pharmaceutical composition comprising a pharmaceutically acceptable excipient and a compound of Formula XXVIII, 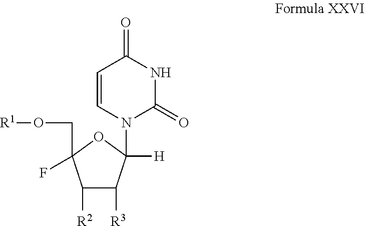
or a pharmaceutical salt thereof, wherein
R¹ is selected from, 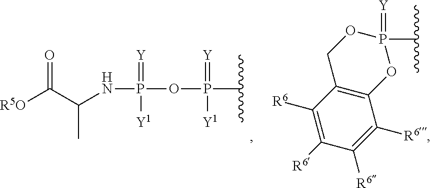 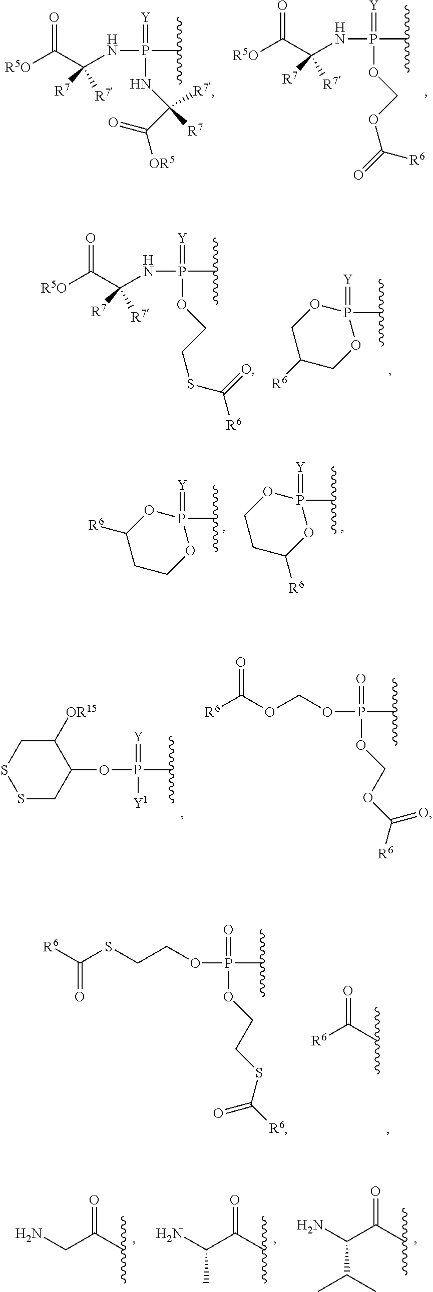 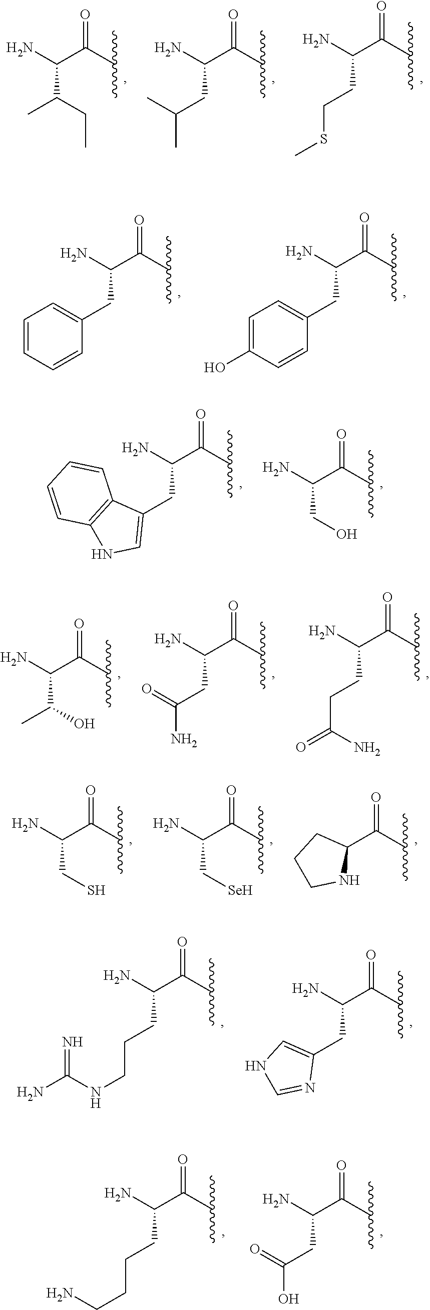 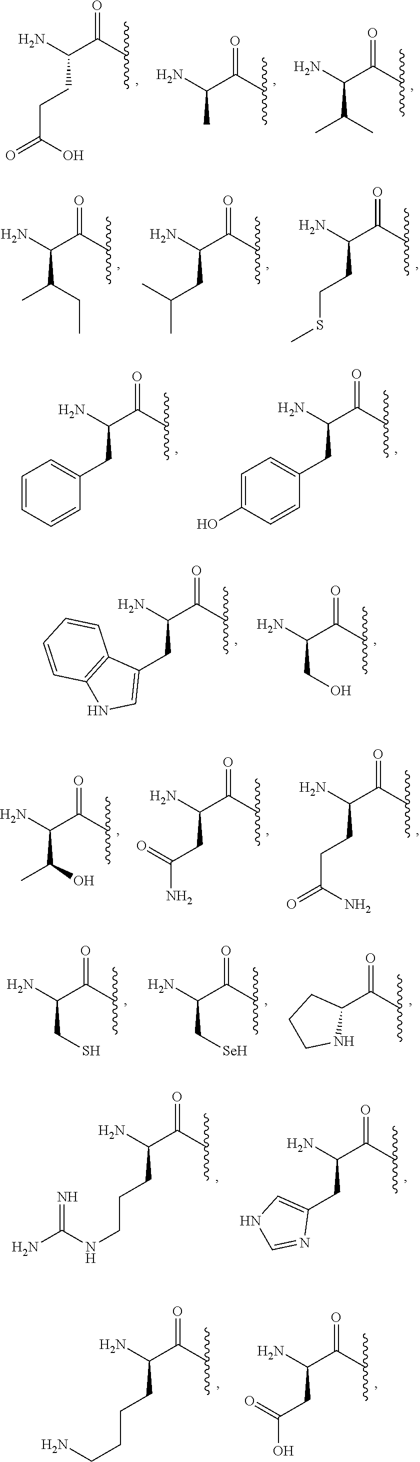 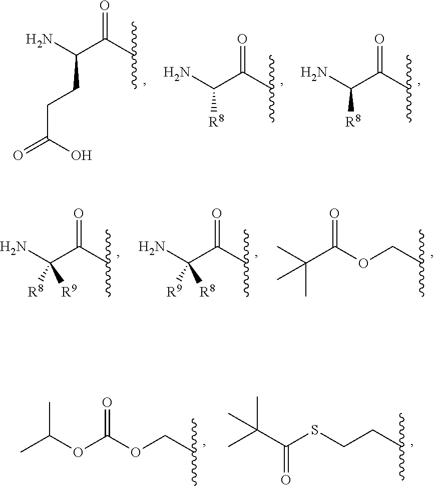
optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thioca, wherein R¹ is optionally substituted with one or more, the same or different, R¹⁰;
Y is O or S;
Y¹ is OH, OY³, or BH₃⁻M⁺;
Y³ is aryl, heteroaryl, or heterocyclyl, wherein Y³ is optionally substituted with one or more, the same or different, R¹⁰;
R² is selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R² is optionally substituted with one or more, the same or different, R¹⁰;
R³ is selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R³ is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and
R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁸ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁸ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon to which they are attached and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein
wherein the medicament is administered to the subject greater than or equal to 3 days after the patient presents with clinical signs of disease.

398. The method of claim 397, wherein R² and R³ are hydroxy.

399. The method of claim 398, wherein R¹ has the formula: 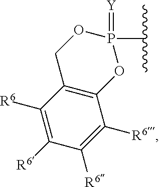
Y is O; and
R⁶, R^{6′}, R^{6″}, and R^{6″′} are each independently selected from hydrogen, hydroxyl, halogen, nitro, alkyl, and alkoxy.

400. The method of claim 399, wherein the compound has the formula:

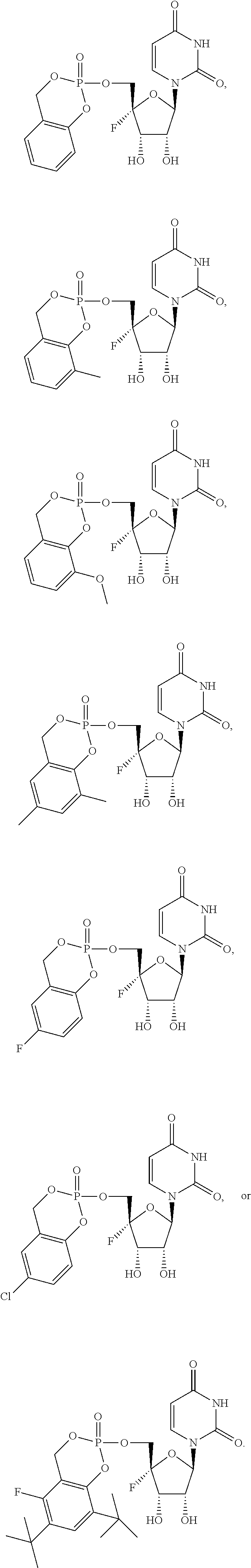

401. The method of claim 400, wherein the compound has the formula:

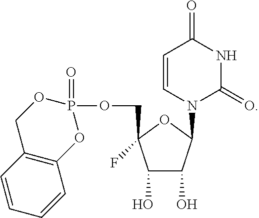 [SMILES: O=c1ccn([C@@H]2O[C@](F)(COP3(=O)OCc4ccccc4O3)[C@@H](O)[C@H]2O)c(=O)[nH]1]

402. The method of claim 397, wherein R¹ is: 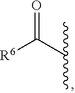
wherein R⁶ is alkyl.

403. The method of claim 402, wherein R² and R³ alkoxy, substituted with one or more, the same or different, R¹⁰; wherein R¹⁰ is in each case independently alkyl or carbonyl.

404. The method of claim 397, wherein the compound is delivered to the CNS of the subject.

405. The method of claim 397, wherein the viral infection is encephalitic.

406. The method of claim 397, wherein viral infection is a SARS-CoV-2 infection.

407. The method of claim 397, further comprising administering an additional antiviral agent to the subject.

408. The method of claim 407, wherein the additional antiviral agent is selected from abacavir, acyclovir, acyclovir, adefovir, amantadine, amprenavir, rintatolimod, arbidol, AT-511, AT-527, atazanavir, atripla, balapiravir, BCX4430/galidesivir, boceprevir, cidofovir, combivir, daclatasvir, darunavir, dasabuvir, delavirdine, didanosine, ocosanol, edoxudine, efavirenz, emtricitabine, enfuvirtide, entecavir, famciclovir, favipiravir, fomivirsen, fosamprenavir, foscarnet, fosfonet, ganciclovir, GS-41524, GS-5734/remdesivir, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, tenofovir exalidex, tipranavir, trifluridine, trizivir, tromantadine, truvada, valaciclovir, valganciclovir, vicriviroc, vidarabine, viramidine zalcitabine, zanamivir, zidovudine, chloroquine, chloroquine phosphate, hydroxychloroquine, hydroxychloroquine sulfate, APNO1, danoprevir, IFX-1, BXT-25, CYNK-001, tocilizumab, leronlimab, Ii-key, COVID-19 S-Trimer, camrelizumab, thymosin, brilacidin, INO-4800, cobicistat, mRNA-1273, umifenovir, REGN3048, REGN3051, TNX-1800, fingolimod, methylprednisolone, nitazoxanide, benzopurpin B, C-467929, C-473872, NSC-306711, N-65828, C-21, CGP-42112A, L-163491, xanthoangelol, bevacizumab, and combinations thereof.

409. A method of treating a SAVS-CoV-2 encephalitic infection in a subject in need thereof, comprising delivering to the brain of the subject a compound having the structure: 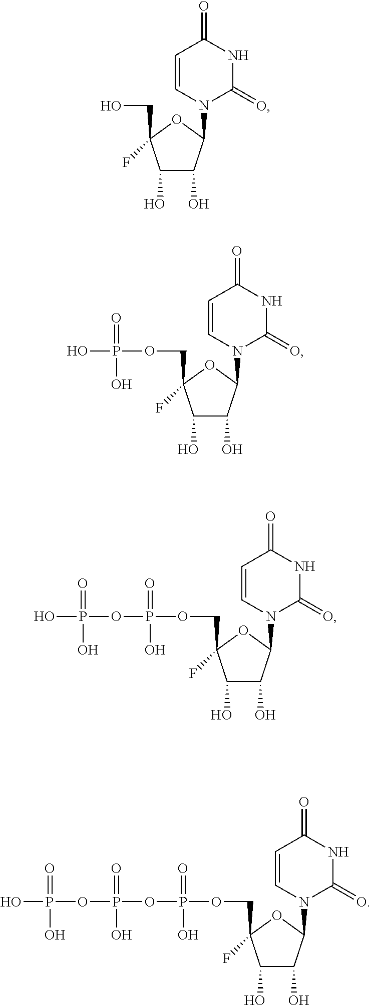 [SMILES: O=c1ccn([C@@H]2O[C@](F)(CO)[C@@H](O)[C@H]2O)c(=O)[nH]1 ; O=c1ccn([C@@H]2O[C@](F)(COP(=O)(O)O)[C@@H](O)[C@H]2O)c(=O)[nH]1 ; O=c1ccn([C@@H]2O[C@](F)(COP(=O)(O)OP(=O)(O)O)[C@@H](O)[C@H]2O)c(=O)[nH]1 ; O=c1ccn([C@@H]2O[C@](F)(COP(=O)(O)OP(=O)(O)OP(=O)(O)O)[C@@H](O)[C@H]2O)c(=O)[nH]1]
or a pharmaceutically acceptable salt thereof.

410. A method of treating a SARS-CoV-2 infection in a subject in need thereof, comprising administering to the subject a pharmaceutical composition comprising a pharmaceutically acceptable excipient and a compound of Formula XXVIII, 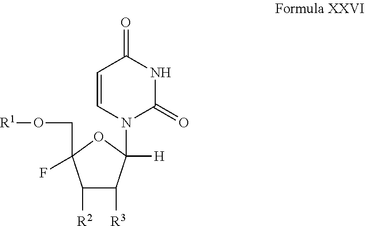
or a pharmaceutical salt thereof, wherein
R¹ is selected from, 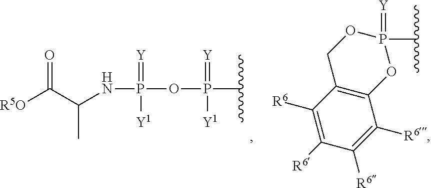 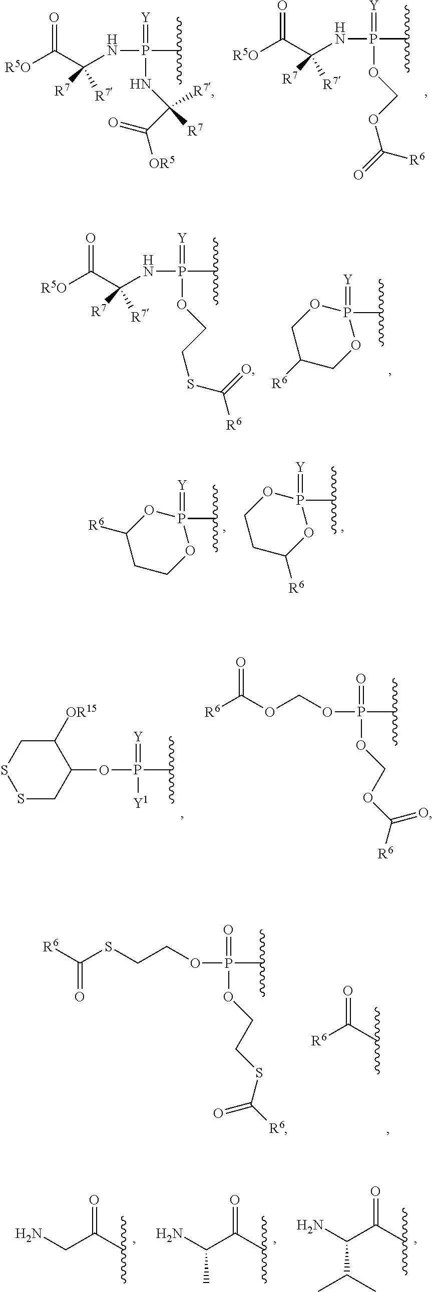 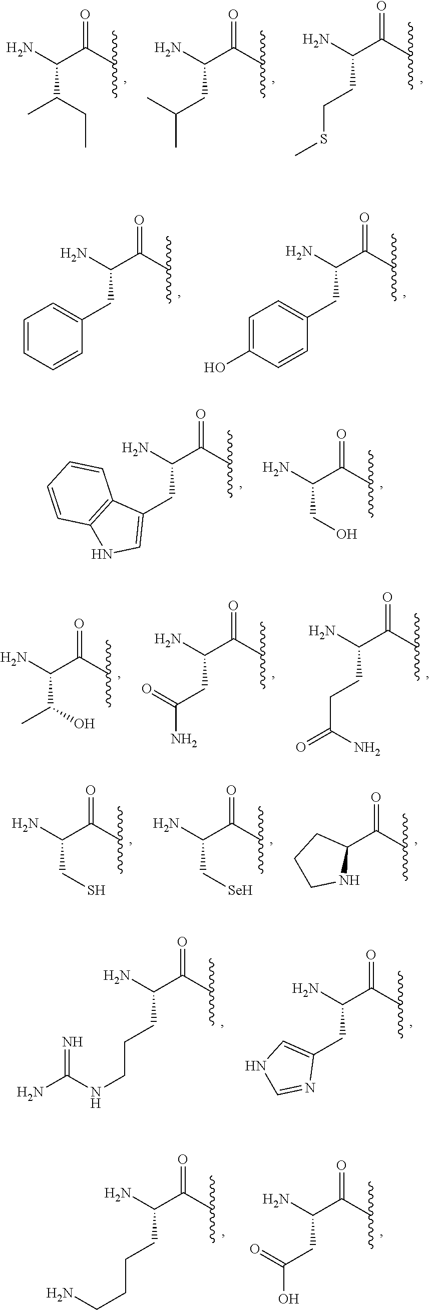 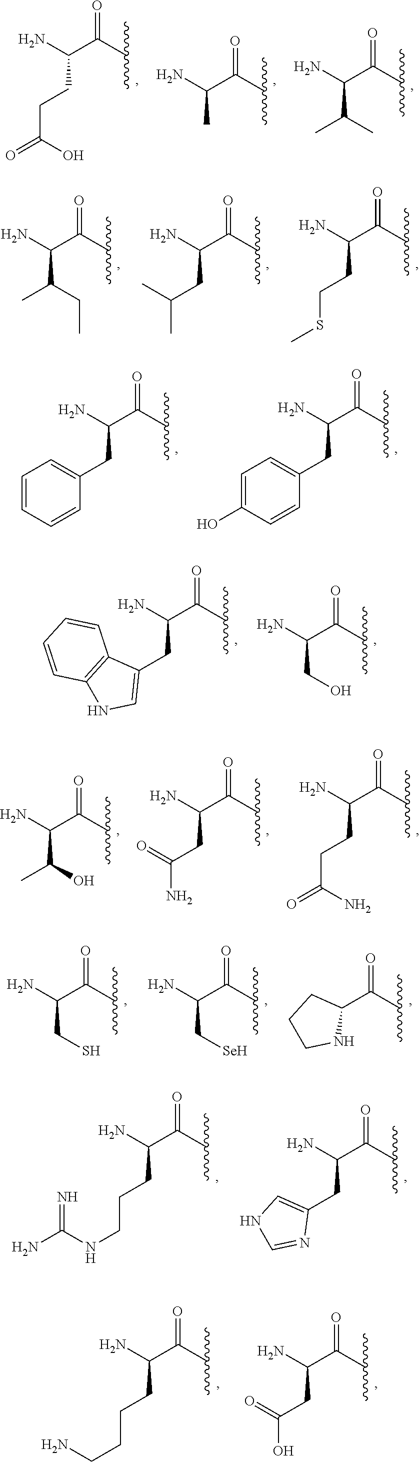 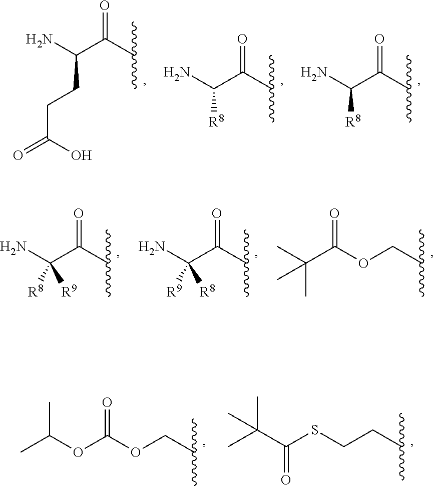
optionally substituted esters, optionally substituted branched esters, optionally substituted carbonates, optionally substituted carbamates, optionally substituted thioesters, optionally substituted branched thioesters, optionally substituted thiocarbonates, optionally substituted S-thiocarbonate, optionally substituted dithiocarbonates, optionally substituted thiocarbamates, optionally substituted oxymethoxycarbonyl, optionally substituted oxymethoxythiocarbonyl, optionally substituted oxymethylcarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, optionally substituted sulfenyl, optionally substituted imidate, optionally substituted hydrazonate, optionally substituted oximyl, optionally substituted imidinyl, optionally substituted imidyl, optionally substituted aminal, optionally substituted hemiaminal, optionally substituted acetal, optionally substituted hemiacetal, optionally substituted carbonimidate, optionally substituted thiocarbonimidate, optionally substituted carbonimidyl, optionally substituted carbamimidate, optionally substituted carbamimidyl, optionally substituted thioacetal, optionally substituted S-acyl-2-thioethyl, optionally substituted bis-(acyloxybenzyl)esters, optionally substituted (acyloxybenzyl)esters, esters, branched esters, carbonates, carbamates, thioesters, branched thioesters, thiocarbonates, sulfenyl thiocarbonates, optionally substituted sulfenyl thiocarbonates, 2-hydroxypropanoate ester, optionally substitute 2-hydroxypropanoate ester, S-thioca, wherein R¹ is optionally substituted with one or more, the same or different, R¹⁰;
Y is O or S;
Y¹ is OH, OY³, or BH₃⁻M⁺;
Y³ is aryl, heteroaryl, or heterocyclyl, wherein Y³ is optionally substituted with one or more, the same or different, R¹⁰;
R² is selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R² is optionally substituted with one or more, the same or different, R¹⁰;
R³ is selected from hydrogen, deuterium, alkyl, alkenyl, alkynyl, allenyl, alkoxy, hydroxy, thiol, amino, azido, formyl, acyl, cyano, halogen, aryl, heteroaryl, heterocyclyl, carbocyclyl, heterocarbocyclyl, sulfinyl, sulfamoyl, or sulfonyl, wherein R³ is optionally substituted with one or more, the same or different, R¹⁰;
R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid, wherein R⁵ is optionally substituted with one or more, the same or different, R¹⁰;
R⁶, R^{6′}, R^{6″}, and R^{6″′} are each independently selected from hydrogen, deuterium, hydroxyl, amino, azido, thiol, acyl, formyl, halogen, nitro, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, sulfinyl, sulfamoyl, sulfonyl allenyl, cyano, or lipid, wherein R⁶, R^{6′}, R^{6″}, and R^{6″′} can each be optionally substituted with one or more, the same or different, R¹⁰;
R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁹ is optionally substituted with one or more, the same or different, R¹⁰;
R⁷, R^{7′}, R⁸, and R⁹ can form a ring with the α-carbon to which they are attached and the amino group attached to the α-carbon, wherein the ring is optionally substituted with one or more, the same or different, R¹⁰;
R⁷ and R^{7′} can form a ring with the α-carbon which they are attached, wherein the ring is optionally substituted with one or more, the same or different, R¹⁰;
R⁸ and R⁹ can form a ring with the α-carbon which they are attached, wherein the ring is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and
Lipid is a C₁₁-C₂₂ higher alkyl, C₁₁-C₂₂ higher alkoxy, polyethylene glycol, or aryl substituted with an alkyl group, or a lipid as described herein.

411. The method of claim 410, wherein R² and R³ are hydroxy.

412. The method of claim 411, wherein R¹ has the formula: 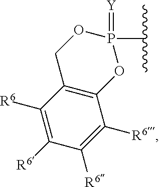
Y is O; and
R⁶, R^{6′}, R^{6″}, and R^{6″′} are each independently selected from hydrogen, hydroxyl, halogen, nitro, alkyl, and alkoxy.

413. The method of claim 412, wherein the compound has the formula:

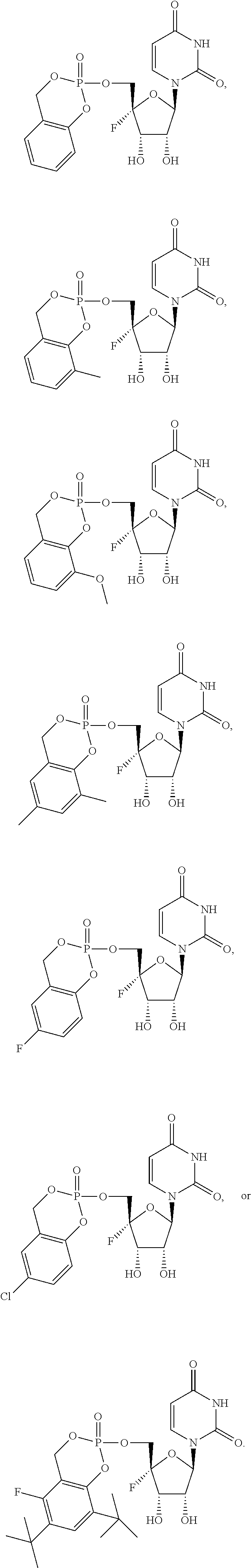

414. The method of claim 413, wherein the compound has the formula:

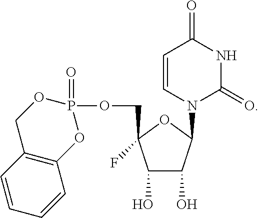 [SMILES: O=c1ccn([C@@H]2O[C@](F)(COP3(=O)OCc4ccccc4O3)[C@@H](O)[C@H]2O)c(=O)[nH]1]

415. The method of claim 410, wherein R¹ is: 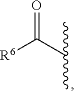
wherein R⁶ is alkyl.

416. The method of claim 415, wherein R² and R³ alkoxy, substituted with one or more, the same or different, R¹⁰; wherein R¹⁰ is in each case independently alkyl or carbonyl.

417. The method of claim 410, wherein the compound is delivered to the CNS of the subject.

418. The method of claim 410, wherein the viral infection is encephalitic.

419. The method of claim 410, wherein the composition is first administered to a patient greater than or equal to 3 days after a patient presents with clinical signs of infection.

420. The method of claim 410, further comprising administering an additional antiviral agent to the subject.

421. The method of claim 420, wherein the additional antiviral agent is selected from abacavir, acyclovir, acyclovir, adefovir, amantadine, amprenavir, rintatolimod, arbidol, AT-511, AT-527, atazanavir, atripla, balapiravir, BCX4430/galidesivir, boceprevir, cidofovir, combivir, daclatasvir, darunavir, dasabuvir, delavirdine, didanosine, ocosanol, edoxudine, efavirenz, emtricitabine, enfuvirtide, entecavir, famciclovir, favipiravir, fomivirsen, fosamprenavir, foscarnet, fosfonet, ganciclovir, GS-41524, GS-5734/remdesivir, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, tenofovir exalidex, tipranavir, trifluridine, trizivir, tromantadine, truvada, valaciclovir, valganciclovir, vicriviroc, vidarabine, viramidine zalcitabine, zanamivir, zidovudine, chloroquine, chloroquine phosphate, hydroxychloroquine, hydroxychloroquine sulfate, APNO1, danoprevir, IFX-1, BXT-25, CYNK-001, tocilizumab, leronlimab, li-key, COVID-19 S-Trimer, camrelizumab, thymosin, brilacidin, INO-4800, cobicistat, mRNA-1273, umifenovir, REGN3048, REGN3051, TNX-1800, fingolimod, methylprednisolone, nitazoxanide, benzopurpin B, C-467929, C-473872, NSC-306711, N-65828, C-21, CGP-42112A, L-163491, xanthoangelol, bevacizumab, and combinations thereof.
